(19) United States
(12) Patent Application Publication
(10) Pub. No.: US 2025/0263424 A1
(43) Pub. Date: Aug. 21, 2025

(54) KRAS INHIBITORS

(71) Applicant: Bristol-Myers Squibb Company, Princeton, NJ (US)

(72) Inventors: Matthew Leo CONDAKES, Brookline, MA (US); Rita Lee CIVIELLO, Killingworth, CT (US); Joanne Jewett BRONSON, Wenham, MA (US); Michael F. PARKER, Higganum, CT (US); Brian Edward FINK, Yardley, PA (US)

(21) Appl. No.: 19/103,040

(22) PCT Filed: Aug. 10, 2023
(86) PCT No.: PCT/US2023/072012

§ 371 Date: Feb. 11, 2025

Related U.S. Application Data
(60) Provisional application No. 63/483,876, filed on Feb. 8, 2023.
(60) Provisional application No. 63/371,191, filed on Aug. 11, 2022.

(51) Int. Cl. C07D 519/00; A61K 31/517; A61K 31/551; C07D 401/14; C07D 403/14; C07D 417/14; C07D 471/08; C07D 487/04; C07D 487/08

(52) U.S. Cl. CPC C07D 519/00; A61K 31/517; A61K 31/551; C07D 401/14; C07D 403/14; C07D 417/14; C07D 471/08; C07D 487/04; C07D 487/08

(57) ABSTRACT

The present disclosure provides KRAS inhibitors. Methods of treating cancers using the compounds are also provided.

### CROSS-REFERENCE TO RELATED APPLICATIONS

This application claims the priority benefit of U.S. Provisional Application Nos. 63/371,191, filed Aug. 11, 2022 and 63/483,876, filed Feb. 8, 2023, both of which are incorporated by reference herein in their entireties.

### FIELD

The present disclosure provides KRAS inhibitors. Methods of treating cancers using the inhibitors are also provided.

### BACKGROUND

The KRAS oncogene is a member of the RAS family of GTPases that are involved in numerous cellular signaling processes. KRAS mutations are gain-of-function mutations that are present in up to 30% of all tumors, including as many as 90% of pancreatic cancers. KRAS serves as a molecular switch cycling between inactive (GDP-bound) and active (GTP-bound) states to transduce upstream cellular signals received from multiple tyrosine kinases to downstream effectors to regulate a wide variety of processes, including cellular proliferation. Single nucleotide substitutions that result in missense mutations at codons 12 and 13 of the KRAS primary amino acid sequence comprise approximately 40% of KRAS driver mutations in lung adenocarcinoma, with a G12C transversion being the most common activating mutation. KRAS G12C mutations occur in about 13% of lung adenocarcinomas and about 3% of colorectal adenocarcinomas and are also present in cancers of the breast, bladder, cervix, ovaries, pancreas and uterus.

Despite several unsuccessful efforts to target KRAS, compounds that inhibit KRAS activity, including those that disrupt effectors such as guanine nucleotide exchange factors and target KRAS G12C, are highly desirable. Clearly there remains a continued interest and effort to develop inhibitors of KRAS, particularly inhibitors of activating KRAS mutants, such as KRAS G12C.

### SUMMARY

The present disclosure is based, in part, on the discovery that unlike other KRAS G12C inhibitors, compounds of the disclosure target the active, KRAS G12C^{(ON)} form of KRAS G12C protein. By inhibiting the G12C^{ON} form of KRAS, G12C, it is expected that the claimed compounds will decrease a cancer's resistance to KRAS G12C inhibition and/or demonstrate increased potency in the clinic. Without being bound by a theory, the inhibition of G12C^{ON} form of KRAS G12C may be a result of the alkenylcarbonyl group attached to ring A in formula (I).

In a first aspect, the present disclosure provides a compound compound of formula (I):

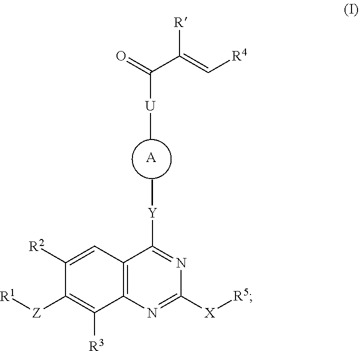

or a pharmaceutically acceptable salt thereof, wherein:
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkoxy, haloC₁-C₃alkyl, hydroxy, and hydroxyC₁-C₃alkyl;
R² and R³ are independently selected from hydrogen, C₁-C₃alkoxy, C₁-C₃alkyl, cyano, halo, haloC₁-C₃alkyl, —C(O)NH₂, —C(O)NH(C₁-C₃alkyl), —C(O)N(C₁-C₃alkyl)₂, and hydroxy;
U is a bond, CH₂NH, or NH;
Y is a bond, O, NR^{g}(CR^{e}R^{f})ₘ, NR^{f}, or CR^{e}R^{f}, wherein m is 1, 2, or 3, and wherein R^{e}, R^{f}, and R^{g} are independently hydrogen or C₁-C₃alkyl;
A is a four- to ten-membered monocyclic or a bicyclic or tricyclic bridged, fused, or spirocyclic saturated, unsaturated, or partially unsaturated ring system optionally containing one or two heteroatoms independently selected from nitrogen, oxygen, and sulfur, wherein the ring system is optionally substituted with one, two, or three groups independently selected from C₁-C₃alkoxy, C₁-C₃alkoxyC₁-C₃alkyl, C₁-C₃alkyl, amidoC₁-C₃alkyl, cyano, cyanoC₁-C₃alkyl, halo, haloC₁-C₃alkyl, amino, aminoC₁-C₃alkyl, hydroxy, hydroxyC₁-C₃alkyl, and oxo; provided that when Y is a bond, A contains at least one nitrogen atom;
R′ is halo;
R⁴ is an aryl or heteroaryl ring optionally substituted with one, two, or three substituents independently selected from C₂-C₄alkenyl, C₁-C₃alkyl, cyano, cyanoC₁-C₃alkyl, halo, haloC₁-C₃alkoxy, haloC₁-C₃alkyl, nitro, and oxo;
X is O or NR¹⁶, wherein R¹⁶ is hydrogen or C₁-C₃alkyl;
R⁵ is selected from hydrogen, C₁-C₆alkoxyC₁-C₆alkyl, C₁-C₆alkyl, aryl, arylC₁-C₆alkyl, carboxyC₁-C₆alkyl, C₃-C₆cycloalkyl, C₃-C₆cycloalkylC₁-C₆alkyl, di(C₁-C₃alkyl)aminoC₂-C₆alkyl, haloC₁-C₆alkyl, heteroaryl, heteroarylC₁-C₆alkyl, heterocyclyl, heterocyclylC₁-C₆alkyl, hydroxyC₁-C₆alkyl, NR^{a}R^{b}—C(O)—C₁-C₆alkyl), NR^{a}R^{b}C₁-C₆alkyl, wherein the aryl, the aryl part of the arylC₁-C₆alkyl, the C₃-C₆cycloalkyl, the cycloalkyl part of the C₃-C₆cycloalkylC₁-C₆alkyl, the heteroaryl, the heteroaryl part of the heteroarylC₁-C₆alkyl, the heterocyclyl, the heterocyclyl part of the heterocyclylC₁-C₆alkyl, are optionally substituted with one, two, three, or four groups independently selected from C₁-C₃alkoxy, C₁-C₃alkoxyC₁-C₃alkyl, C₁-C₃alkyl, (C₁-C₆alkyl)amino, (C₁-C₆alkyl)aminoC₁-C₃alkyl, amino, aminoC₁-C₃alkyl, carboxy, cyano, di(C₁-C₆alkyl)amino, di(C₁-C₆alkyl)aminoC₁-C₃alkyl, halo, haloC₁-C₃alkoxy, haloC₁-C₃alkyl, heterocyclyl, heterocyclylC₁-C₃alkyl, hydroxy, hydroxyC₁-C₃alkyl, nitro, and oxo; wherein the heterocyclyl and the heterocyclyl part of the heterocyclylC₁-C₃alkyl is further optionally substituted with one, two, or three groups independently selected from C₁-C₃alkoxy, C₁-C₃alkyl, halo, and haloC₁-C₃alkyl; or
R⁵ and R¹⁶, together with the nitrogen atom to which they are attached, form a heterocyclic group optionally substituted with one, two, three, four, or five groups independently selected from one, two, three, or four groups independently selected from C₁-C₃alkoxy, C₁-C₃alkoxyC₁-C₃alkyl, C₁-C₃alkyl, amino, aminoC₁-C₃alkyl, hydroxy, and hydroxyC₁-C₃alkyl; and
one of R^{a} and R^{b} is selected from hydrogen and C₁-C₃alkyl and the other is selected from hydrogen, C₁-C₃alkyl, C₁-C₃alkoxycarbonyl, C₁-C₃alkylcarbonyl, arylC₁-C₆alkyl, C₃-C₆cycloalkyl, and C₃-C₆cycloalkylC₁-C₆alkyl.

In certain aspects, the present disclosure provides a compound of formula (I), or a pharmaceutically acceptable salt thereof, wherein R⁴ is a five- or six-membered aromatic ring optionally containing one, two, or three heteroatoms independently selected from nitrogen, oxygen, and sulfur.

In certain aspects, the present disclosure provides a compound of formula (I), or a pharmaceutically acceptable salt thereof, wherein Y is a bond.

In certain aspects, the present disclosure provides a compound of formula (I), or a pharmaceutically acceptable salt thereof, wherein Y is NH.

In certain aspects, the present disclosure provides a compound of formula (I), or a pharmaceutically acceptable salt thereof, wherein Y is NCH₃.

In certain aspects, the present disclosure provides a compound of formula (I), or a pharmaceutically acceptable salt thereof, wherein A is a six- to ten-membered monocyclic or a bicyclic or tricyclic bridged, fused, or spirocyclic saturated ring system containing one or two nitrogen atoms.

In certain aspects, the present disclosure provides a compound of formula (I), or a pharmaceutically acceptable salt thereof, wherein A is a five- to ten-membered monocyclic or a bicyclic or tricyclic bridged, fused, or spirocyclic saturated ring system containing one or two nitrogen atoms.

In certain aspects, the present disclosure provides a compound of formula (I), or a pharmaceutically acceptable salt thereof, wherein U is a bond. In certain aspects, the present disclosure provides a compound of formula (I), or a pharmaceutically acceptable salt thereof, wherein U is CH₂NH. In certain aspects, the present disclosure provides a compound of formula (I), or a pharmaceutically acceptable salt thereof, wherein U is a NH.

In certain aspects, the present disclosure provides a compound of formula (I), or a pharmaceutically acceptable salt thereof, wherein A-U is selected from:

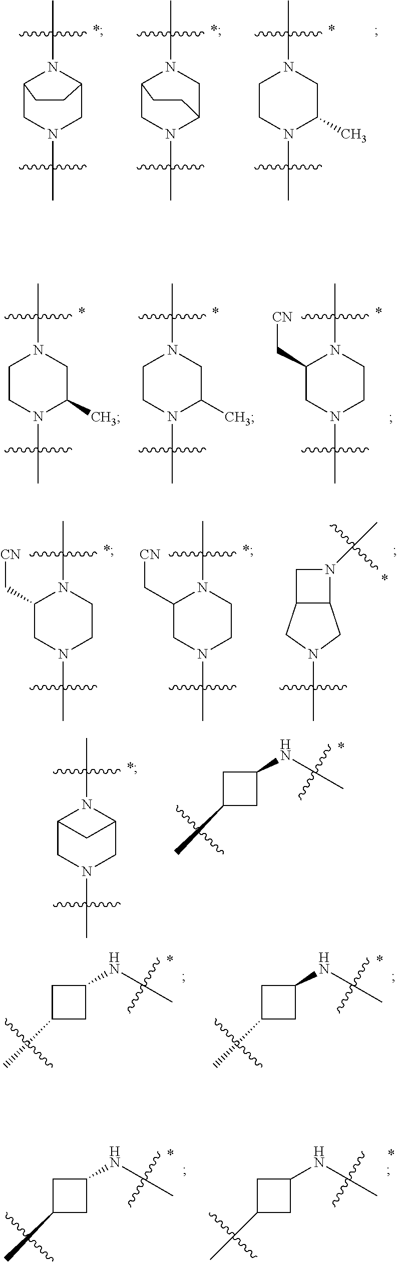

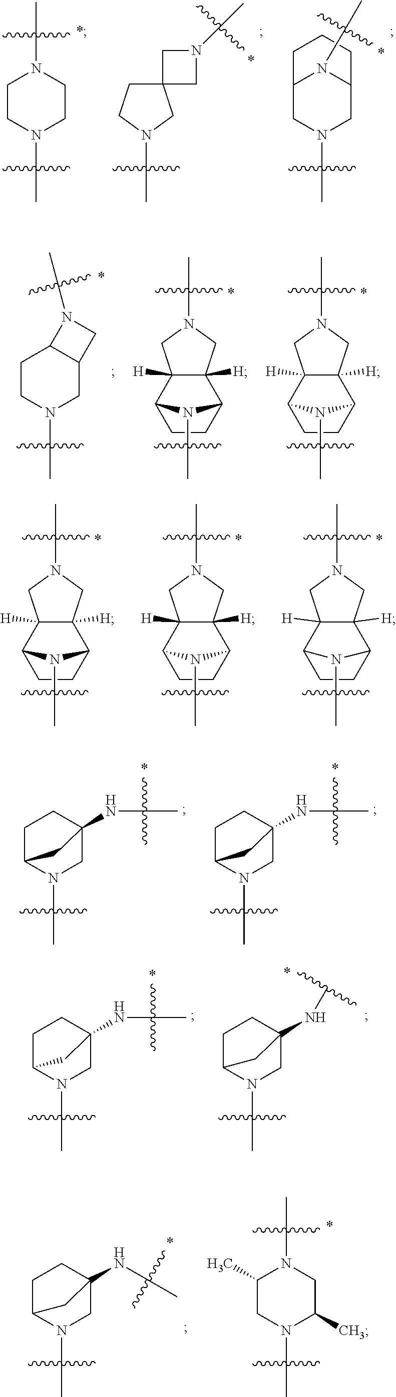

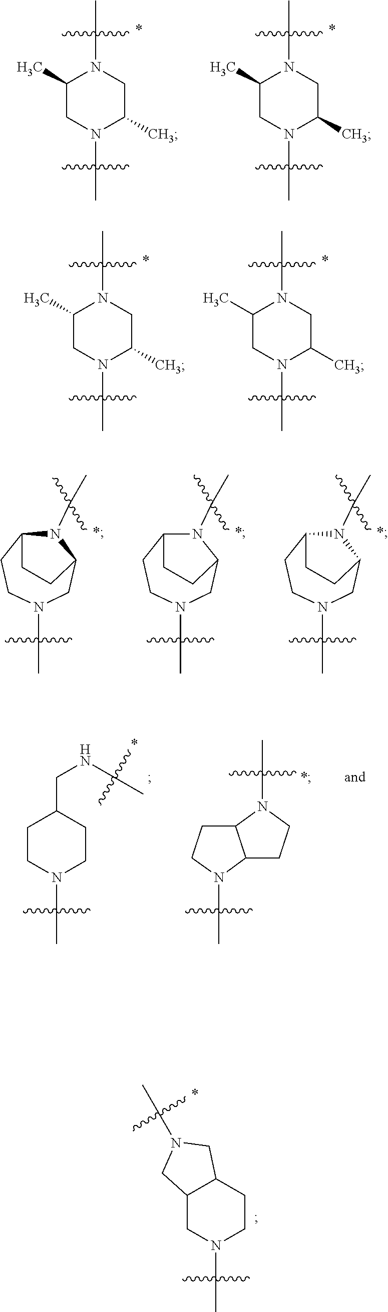

wherein:
[symbol] represents the point of attachment to the carbonyl group; and
[symbol] represents the point of attachment to Y.

In certain aspects, the present disclosure provides a compound of formula (I), or a pharmaceutically acceptable salt thereof, wherein Y-A-U is selected from the group consisting of:

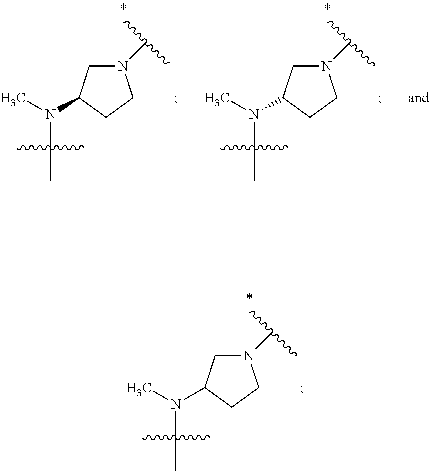

wherein:
[symbol] represents the point of attachment to the carbonyl group; and
[symbol] represents the point of attachment to the quinazoline ring.

In certain aspects, the present disclosure provides a compound of formula (I), or a pharmaceutically acceptable salt thereof, wherein R² is halo.

In certain aspects, the present disclosure provides a compound of formula (I), or a pharmaceutically acceptable salt thereof, wherein R³ is halo.

In certain aspects, the present disclosure provides a compound of formula (I), or a pharmaceutically acceptable salt thereof, wherein R⁴ is selected from isothiazolyl, pyridinyl, pyrimidinyl, and thiazolyl.

In certain aspects, the present disclosure provides a compound of formula (I), or a pharmaceutically acceptable salt thereof, wherein X is O.

In certain aspects, the present disclosure provides a compound of formula (I), or a pharmaceutically acceptable salt thereof, wherein R⁵ is selected from:

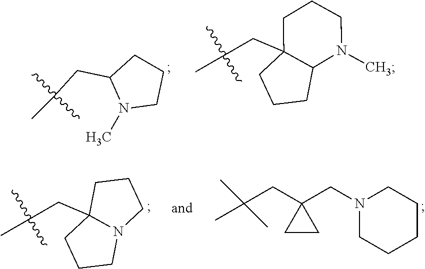

wherein each ring is optionally substituted with 1, 2, or 3 groups independently selected from C₁-C₃alkoxy, C₁-C₃alkoxyC₁-C₃alkyl, C₁-C₃alkyl, benzyl, halo, haloC₁-C₃alkyl, hydroxy, hydroxyC₁-C₃alkyl, and oxo.

In certain aspects, the present disclosure provides a compound of formula (I), or a pharmaceutically acceptable salt thereof, wherein R⁵ is —(C₁-C₃alkyl)-R⁶, wherein R⁶ is a three- to five-membered monocyclic ring system, an eight- or nine-membered bicyclic fused saturated ring system, or a ten-membered tricyclic saturated ring system, wherein each ring system optionally contains one nitrogen atom, and wherein each ring system is optionally substituted with one or two groups independently selected from C₁-C₃alkyl, halo, and (4- to 6-membered heterocyclyl)C₁-C₃alkyl; wherein the heterocyclyl part of the (4- to 6-membered heterocyclyl)C₁-C₃alkyl is further optionally substituted with a halo group.

In certain aspects, the present disclosure provides a compound of formula (I), or a pharmaceutically acceptable salt thereof, wherein R⁵ is

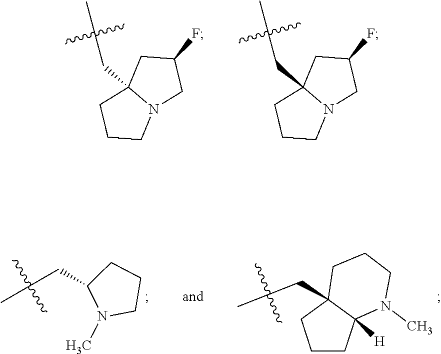

and [symbol] represents the point of attachment to X.

In certain aspects, the present disclosure provides a compound of formula (I), or a pharmaceutically acceptable salt thereof, wherein R⁵ is

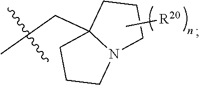

wherein:
n is 0, 1, or 2;
each R²⁰ is halo; and
[symbol] represents the point of attachment to X.

In certain aspects, the present disclosure provides a compound of formula (I), or a pharmaceutically acceptable salt thereof, wherein Z is a bond.

In certain aspects, the present disclosure provides a compound of formula (I), or a pharmaceutically acceptable salt thereof, wherein R¹ is a monocyclic heteroaryl ring containing one, two, or three nitrogen atoms, wherein the ring is optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, hydroxy, and hydroxyC₁-C₃alkyl.

In certain aspects, the present disclosure provides a compound of formula (I), or a pharmaceutically acceptable salt thereof, wherein R¹ is

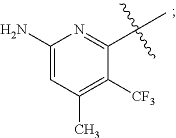

wherein [symbol] denotes the point of attachment to the parent molecular moiety.

In certain aspects, the present disclosure provides a compound of formula (I), or a pharmaceutically acceptable salt thereof, wherein R¹ is C₆-C₁₀aryl optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, hydroxy, and hydroxyC₁-C₃alkyl.

In certain aspects, the present disclosure provides a compound of formula (I), or a pharmaceutically acceptable salt thereof, wherein R¹ is naphtyl substituted with one, two, three, four, or five substituents independently selected from C₁-C₃alkyl, C₂-C₄alkynyl, halo, and hydroxy.

In certain aspects, the present disclosure provides a compound of formula (I), or a pharmaceutically acceptable salt thereof, wherein R¹ is naphthyl, wherein the naphthyl is substituted with one, two, or three groups independently selected from C₂-C₄alkynyl, halo, and hydroxy.

In certain aspects, the present disclosure provides a compound of formula (I), or a pharmaceutically acceptable salt thereof, wherein R¹ is

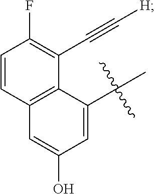

wherein [symbol] denotes the point of attachment to the parent molecular moiety.

In certain aspects, the present disclosure provides a compound of formula (I), or a pharmaceutically acceptable salt thereof, wherein R′ is fluoro.

In certain aspects, the present disclosure provides a compound of formula (I), or a pharmaceutically acceptable salt thereof, wherein R′ is chloro.

In some aspects, the present disclosure provides a compound selected from

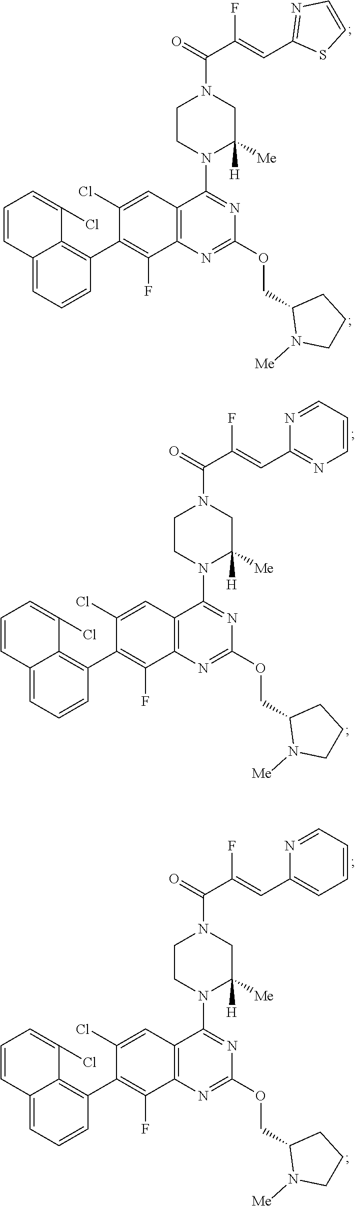 [SMILES: C[C@H]1CN(C(=O)/C(F)=C/c2nccs2)CCN1c1nc(OC[C@@H]2CCCN2C)nc2c(F)c(-c3cccc4cccc(Cl)c34)c(Cl)cc12 ; C[C@H]1CN(C(=O)/C(F)=C/c2ncccn2)CCN1c1nc(OC[C@@H]2CCCN2C)nc2c(F)c(-c3cccc4cccc(Cl)c34)c(Cl)cc12 ; C[C@H]1CN(C(=O)/C(F)=C/c2ccccn2)CCN1c1nc(OC[C@@H]2CCCN2C)nc2c(F)c(-c3cccc4cccc(Cl)c34)c(Cl)cc12]

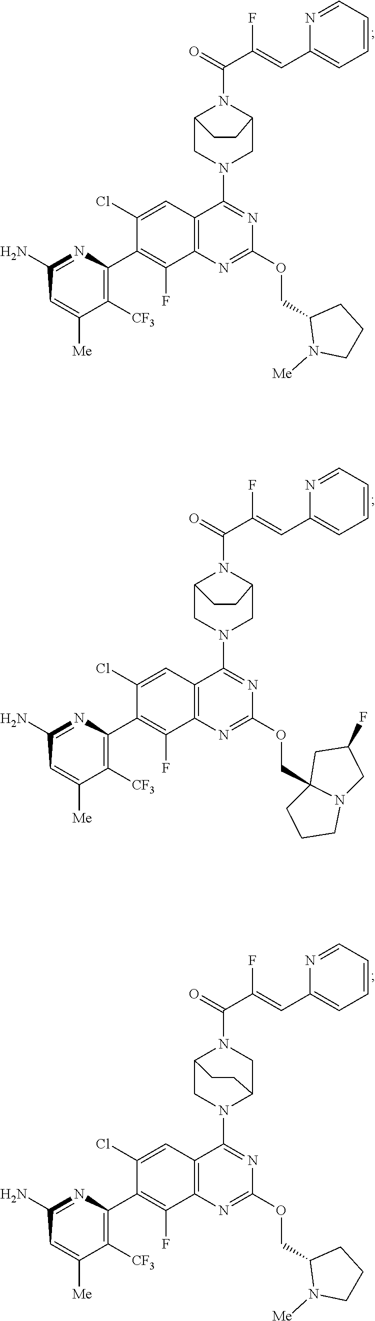

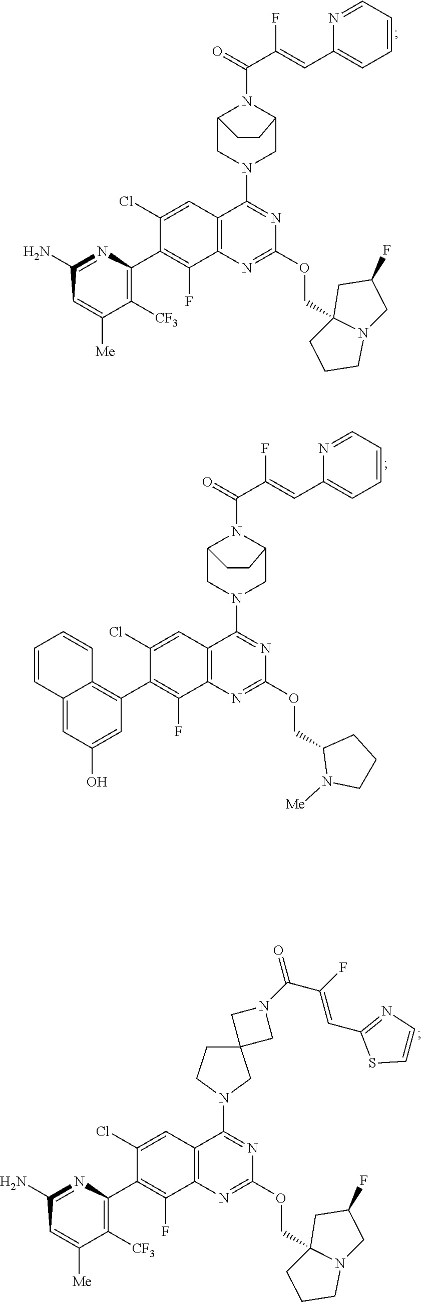

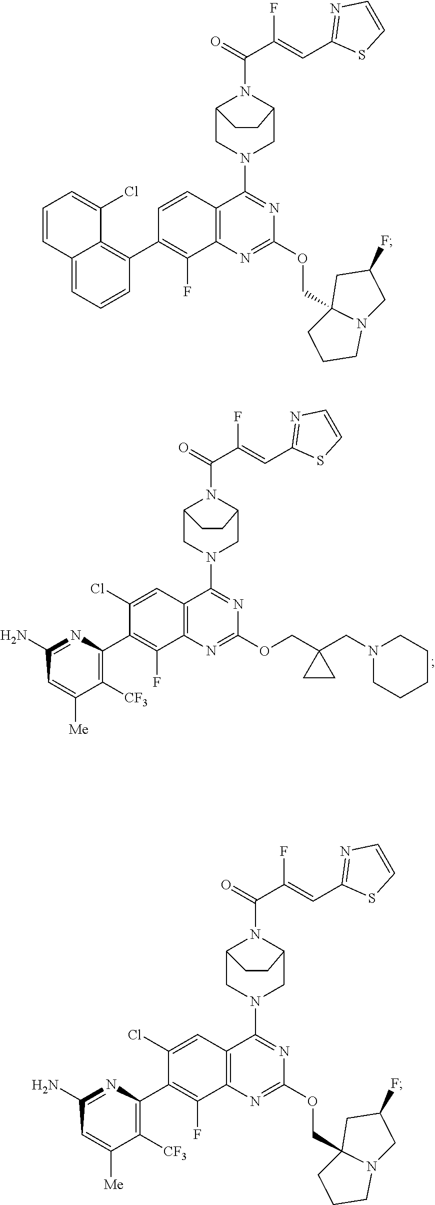

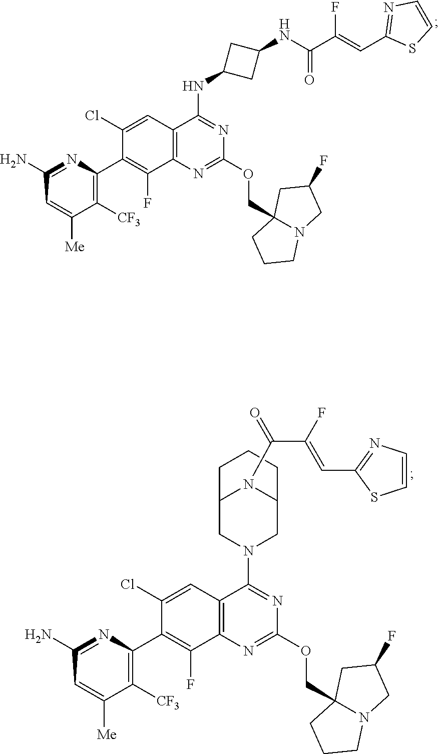

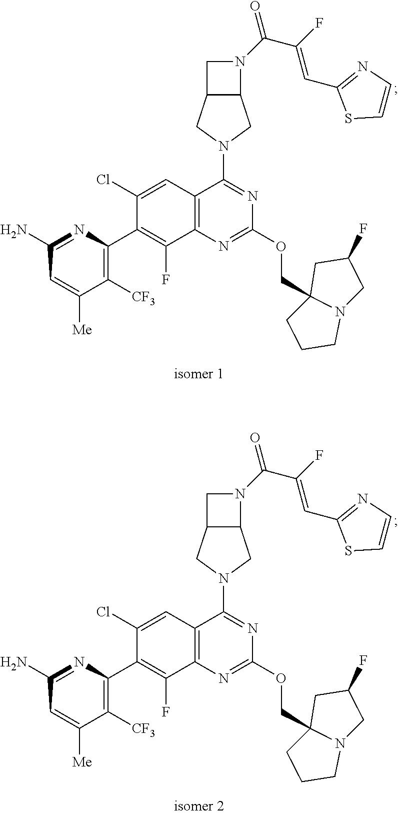

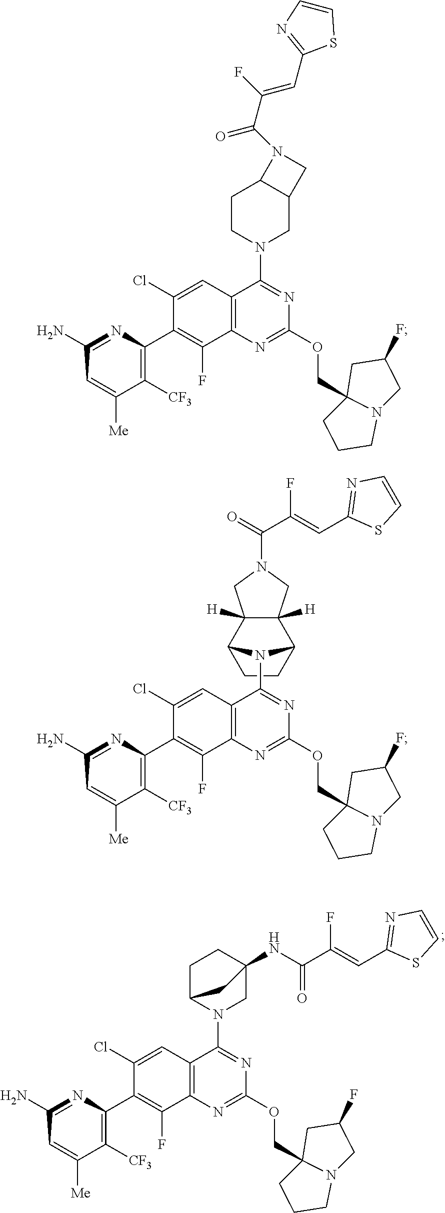

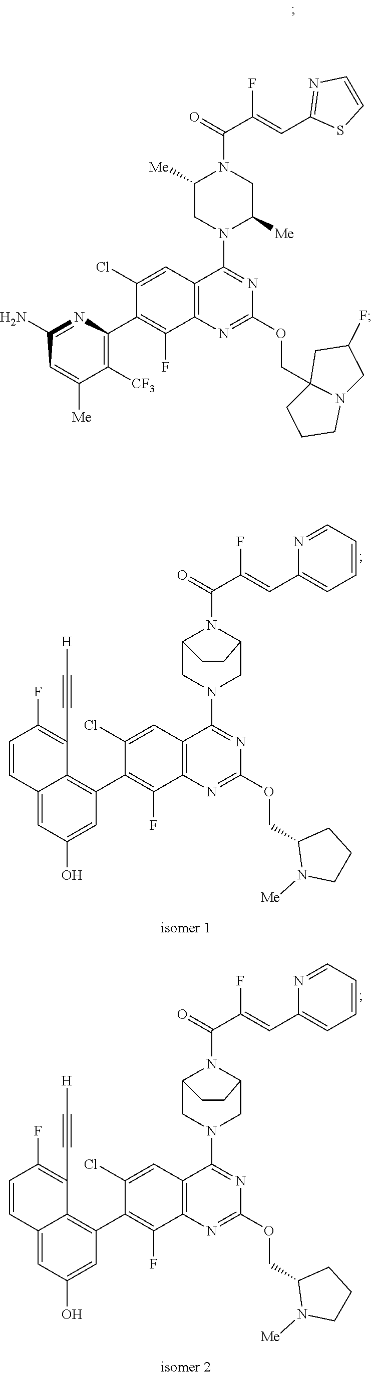

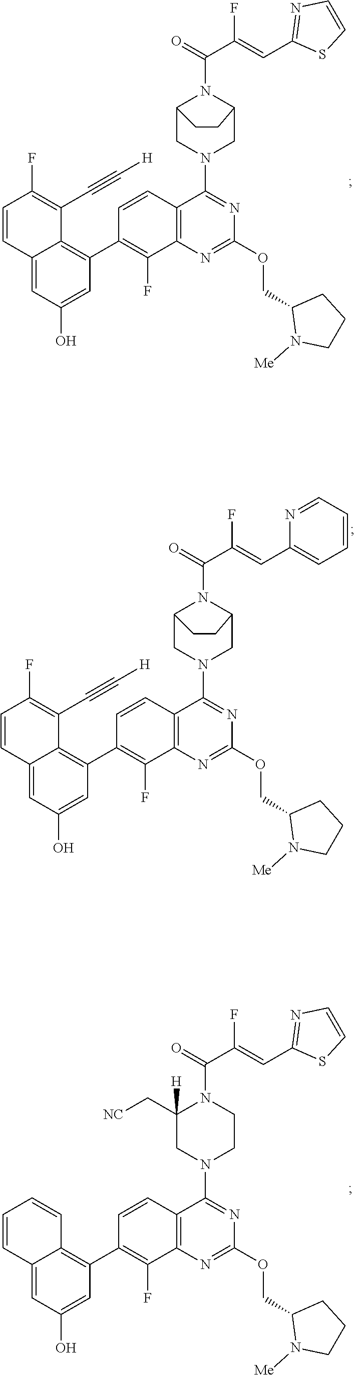 [SMILES: C#Cc1c(F)ccc2cc(O)cc(-c3ccc4c(N5CC6CCC(C5)N6C(=O)/C(F)=C/c5nccs5)nc(OC[C@@H]5CCCN5C)nc4c3F)c12 ; C#Cc1c(F)ccc2cc(O)cc(-c3ccc4c(N5CC6CCC(C5)N6C(=O)/C(F)=C/c5ccccn5)nc(OC[C@@H]5CCCN5C)nc4c3F)c12 ; CN1CCC[C@H]1COc1nc(N2CCN(C(=O)/C(F)=C/c3nccs3)[C@@H](CC#N)C2)c2ccc(-c3cc(O)cc4ccccc34)c(F)c2n1]

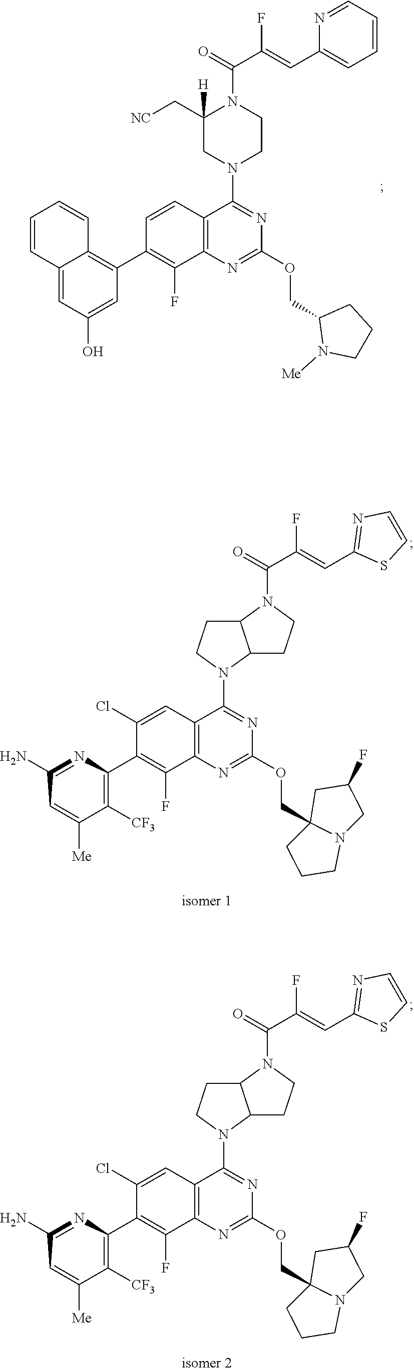

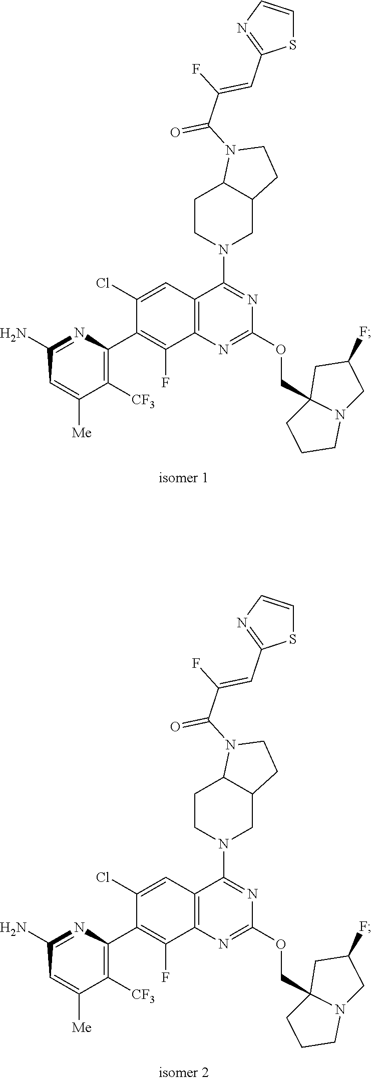

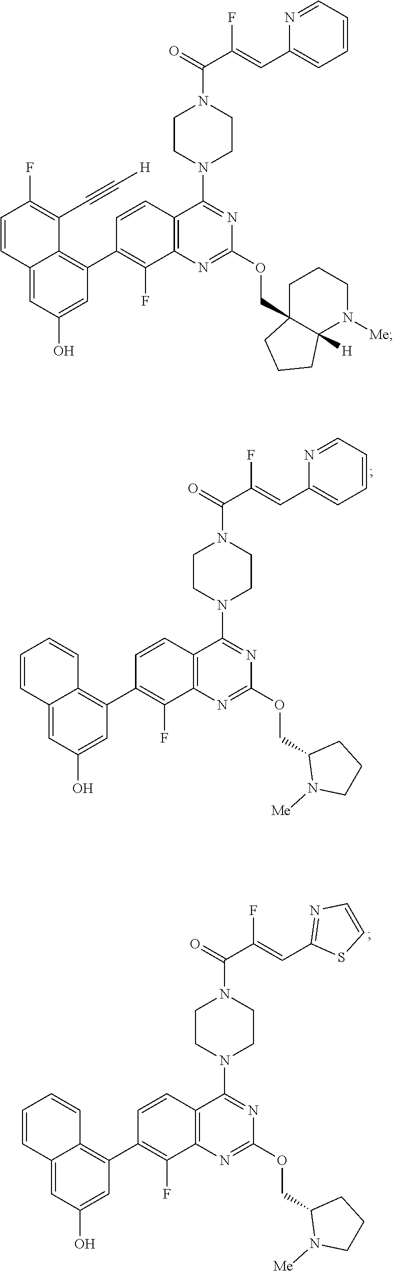 [SMILES: C#Cc1c(F)ccc2cc(O)cc(-c3ccc4c(N5CCN(C(=O)/C(F)=C/c6ccccn6)CC5)nc(OC[C@]56CCC[C@H]5N(C)CCC6)nc4c3F)c12 ; CN1CCC[C@H]1COc1nc(N2CCN(C(=O)/C(F)=C/c3ccccn3)CC2)c2ccc(-c3cc(O)cc4ccccc34)c(F)c2n1 ; CN1CCC[C@H]1COc1nc(N2CCN(C(=O)/C(F)=C/c3nccs3)CC2)c2ccc(-c3cc(O)cc4ccccc34)c(F)c2n1]

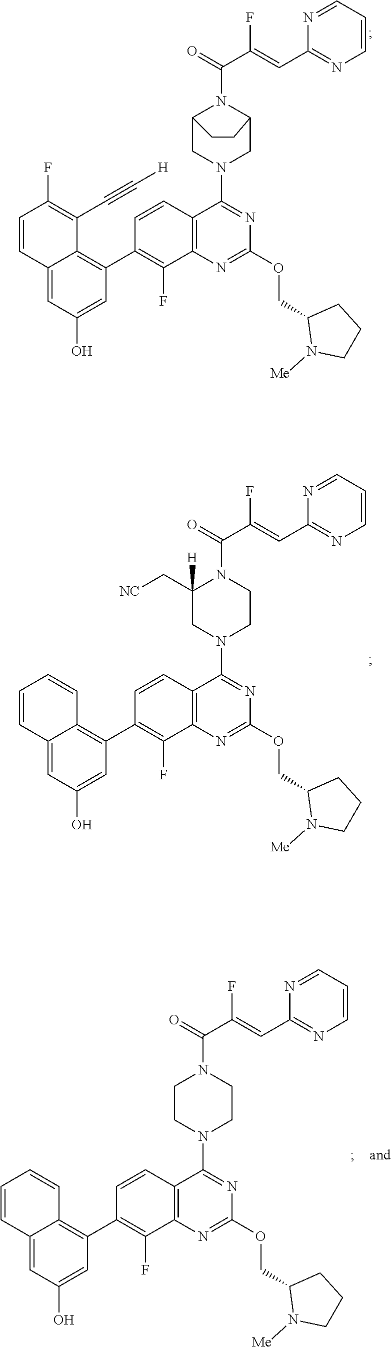 [SMILES: C#Cc1c(F)ccc2cc(O)cc(-c3ccc4c(N5CC6CCC(C5)N6C(=O)/C(F)=C/c5ncccn5)nc(OC[C@@H]5CCCN5C)nc4c3F)c12 ; CN1CCC[C@H]1COc1nc(N2CCN(C(=O)/C(F)=C/c3ncccn3)[C@@H](CC#N)C2)c2ccc(-c3cc(O)cc4ccccc34)c(F)c2n1 ; CN1CCC[C@H]1COc1nc(N2CCN(C(=O)/C(F)=C/c3ncccn3)CC2)c2ccc(-c3cc(O)cc4ccccc34)c(F)c2n1]

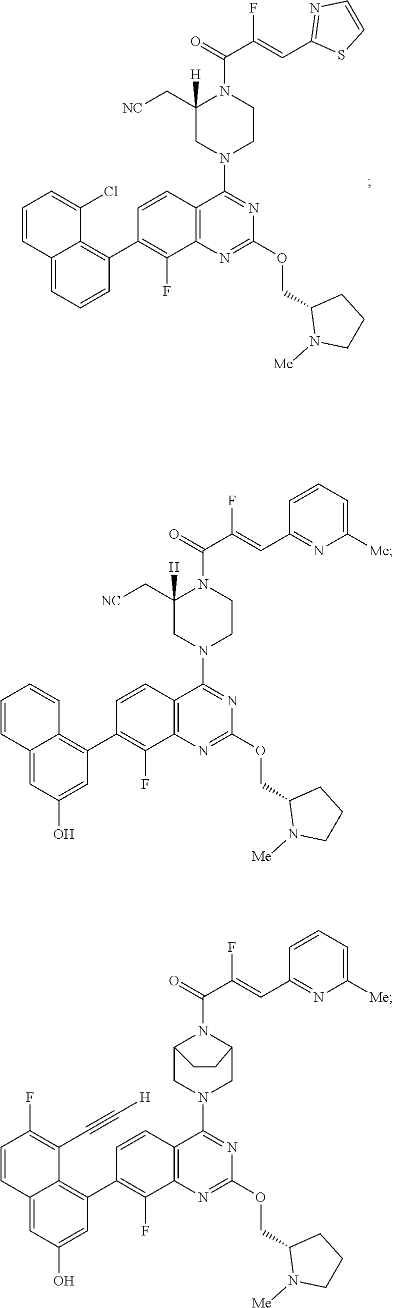 [SMILES: CN1CCC[C@H]1COc1nc(N2CCN(C(=O)/C(F)=C/c3nccs3)[C@@H](CC#N)C2)c2ccc(-c3cccc4cccc(Cl)c34)c(F)c2n1 ; Cc1cccc(/C=C(\F)C(=O)N2CCN(c3nc(OC[C@@H]4CCCN4C)nc4c(F)c(-c5cc(O)cc6ccccc56)ccc34)C[C@@H]2CC#N)n1 ; C#Cc1c(F)ccc2cc(O)cc(-c3ccc4c(N5CC6CCC(C5)N6C(=O)/C(F)=C/c5cccc(C)n5)nc(OC[C@@H]5CCCN5C)nc4c3F)c12]

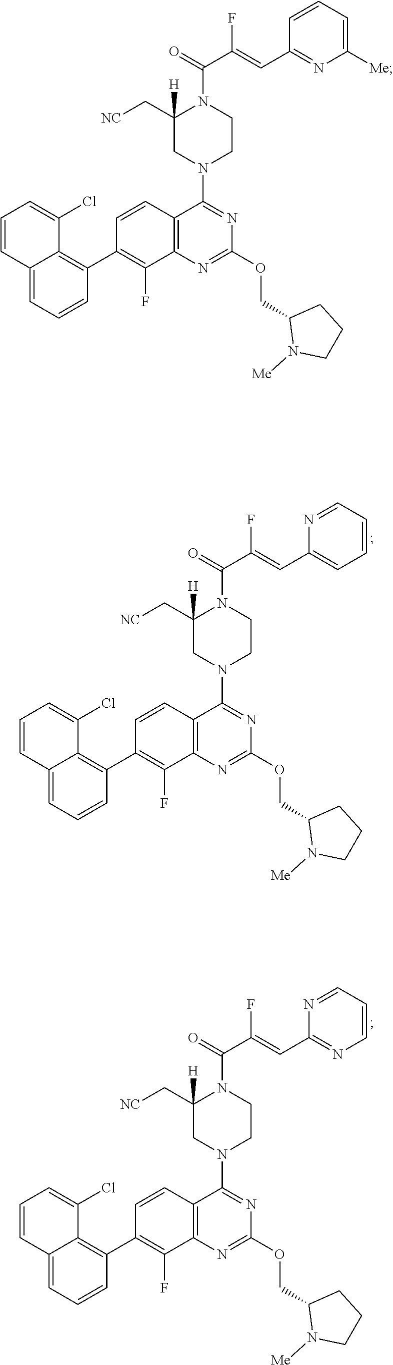 [SMILES: Cc1cccc(/C=C(\F)C(=O)N2CCN(c3nc(OC[C@@H]4CCCN4C)nc4c(F)c(-c5cccc6cccc(Cl)c56)ccc34)C[C@@H]2CC#N)n1 ; CN1CCC[C@H]1COc1nc(N2CCN(C(=O)/C(F)=C/c3ccccn3)[C@@H](CC#N)C2)c2ccc(-c3cccc4cccc(Cl)c34)c(F)c2n1 ; CN1CCC[C@H]1COc1nc(N2CCN(C(=O)/C(F)=C/c3ncccn3)[C@@H](CC#N)C2)c2ccc(-c3cccc4cccc(Cl)c34)c(F)c2n1]

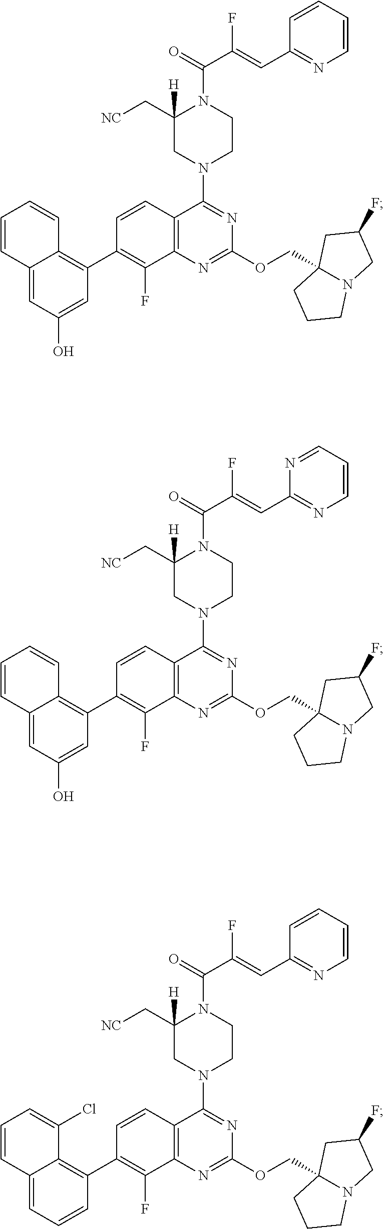 [SMILES: N#CC[C@H]1CN(c2nc(OC[C@@]34CCCN3C[C@H](F)C4)nc3c(F)c(-c4cc(O)cc5ccccc45)ccc23)CCN1C(=O)/C(F)=C/c1ccccn1 ; N#CC[C@H]1CN(c2nc(OC[C@@]34CCCN3C[C@H](F)C4)nc3c(F)c(-c4cc(O)cc5ccccc45)ccc23)CCN1C(=O)/C(F)=C/c1ncccn1 ; N#CC[C@H]1CN(c2nc(OC[C@@]34CCCN3C[C@H](F)C4)nc3c(F)c(-c4cccc5cccc(Cl)c45)ccc23)CCN1C(=O)/C(F)=C/c1ccccn1]

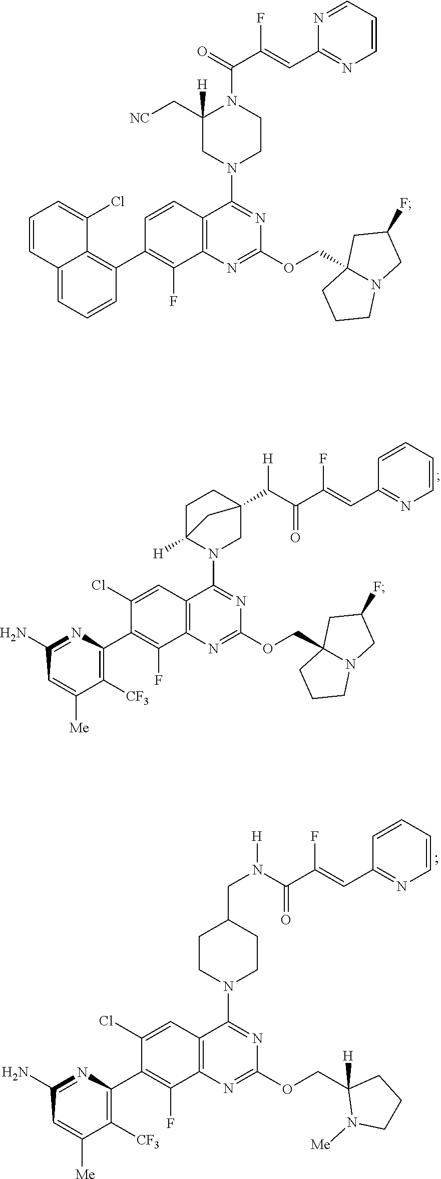

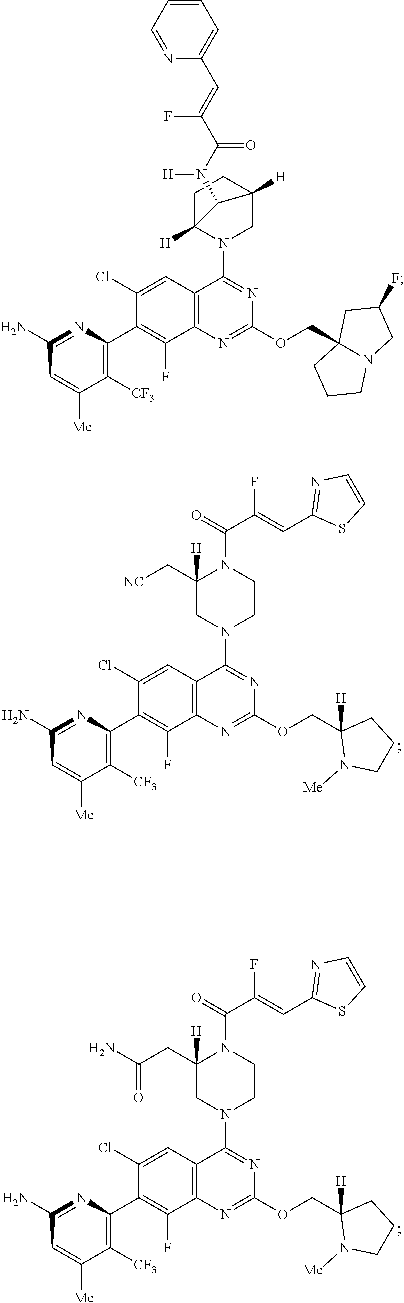

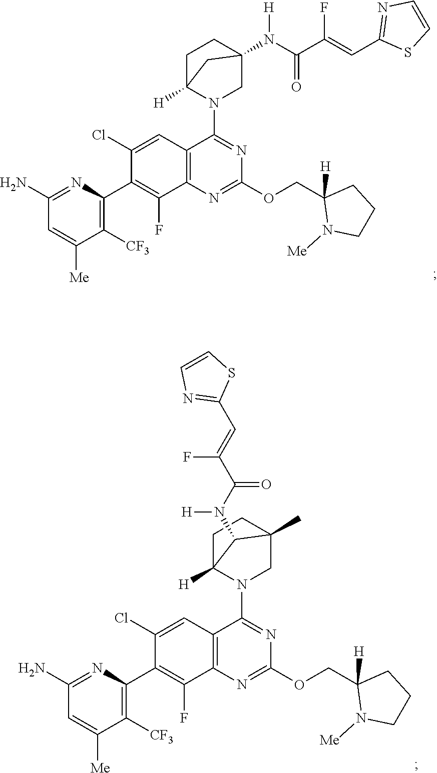

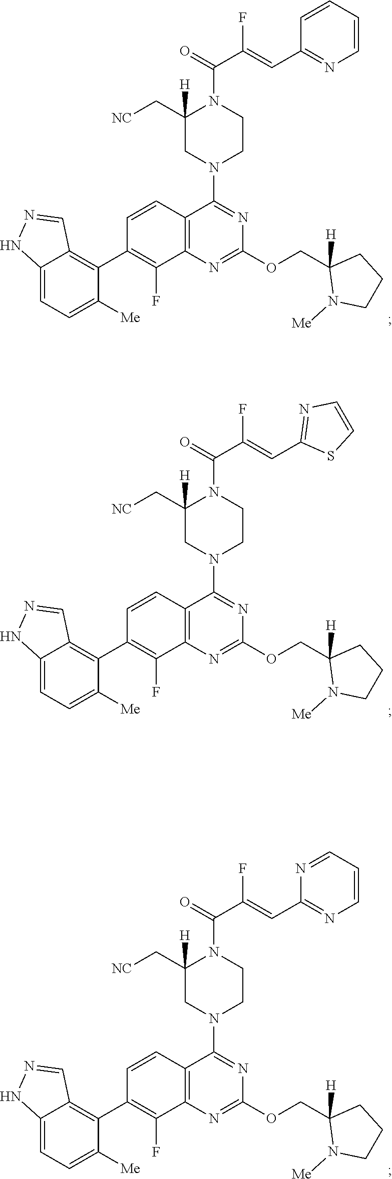 [SMILES: Cc1ccc2[nH]ncc2c1-c1ccc2c(N3CCN(C(=O)/C(F)=C/c4ccccn4)[C@@H](CC#N)C3)nc(OC[C@@H]3CCCN3C)nc2c1F ; Cc1ccc2[nH]ncc2c1-c1ccc2c(N3CCN(C(=O)/C(F)=C/c4nccs4)[C@@H](CC#N)C3)nc(OC[C@@H]3CCCN3C)nc2c1F ; Cc1ccc2[nH]ncc2c1-c1ccc2c(N3CCN(C(=O)/C(F)=C/c4ncccn4)[C@@H](CC#N)C3)nc(OC[C@@H]3CCCN3C)nc2c1F]

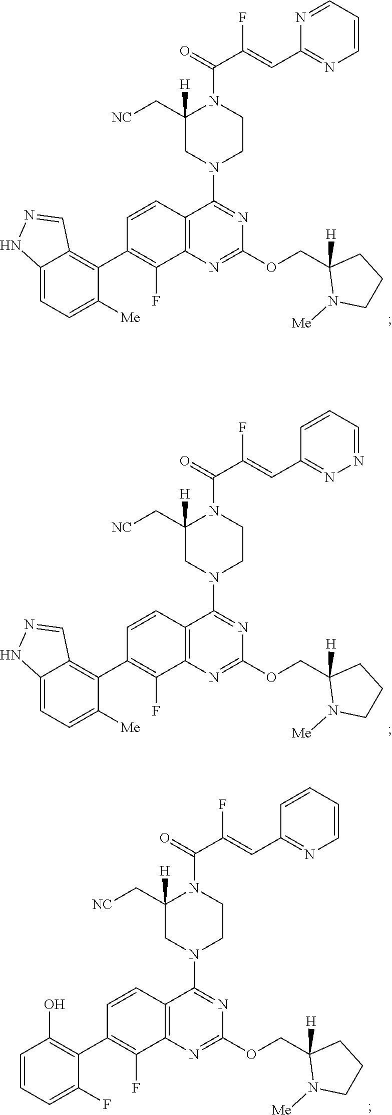 [SMILES: Cc1ccc2[nH]ncc2c1-c1ccc2c(N3CCN(C(=O)/C(F)=C/c4ncccn4)[C@@H](CC#N)C3)nc(OC[C@@H]3CCCN3C)nc2c1F ; Cc1ccc2[nH]ncc2c1-c1ccc2c(N3CCN(C(=O)/C(F)=C/c4cccnn4)[C@@H](CC#N)C3)nc(OC[C@@H]3CCCN3C)nc2c1F ; CN1CCC[C@H]1COc1nc(N2CCN(C(=O)/C(F)=C/c3ccccn3)[C@@H](CC#N)C2)c2ccc(-c3c(O)cccc3F)c(F)c2n1]

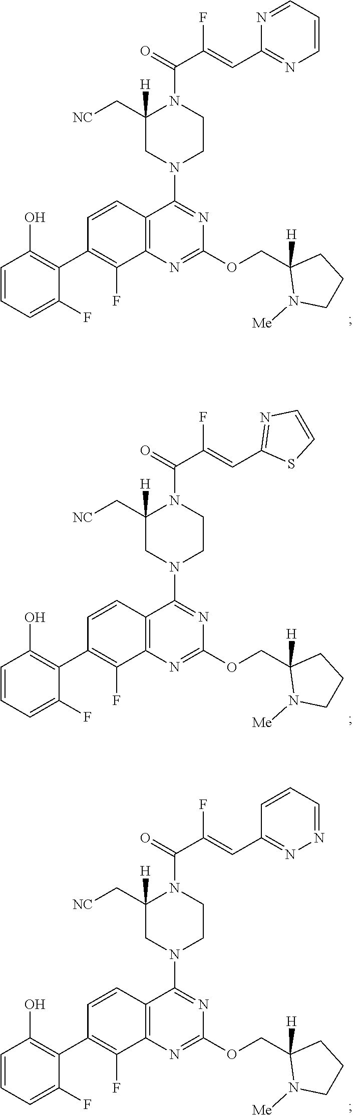 [SMILES: CN1CCC[C@H]1COc1nc(N2CCN(C(=O)/C(F)=C/c3ncccn3)[C@@H](CC#N)C2)c2ccc(-c3c(O)cccc3F)c(F)c2n1 ; CN1CCC[C@H]1COc1nc(N2CCN(C(=O)/C(F)=C/c3nccs3)[C@@H](CC#N)C2)c2ccc(-c3c(O)cccc3F)c(F)c2n1 ; CN1CCC[C@H]1COc1nc(N2CCN(C(=O)/C(F)=C/c3cccnn3)[C@@H](CC#N)C2)c2ccc(-c3c(O)cccc3F)c(F)c2n1]

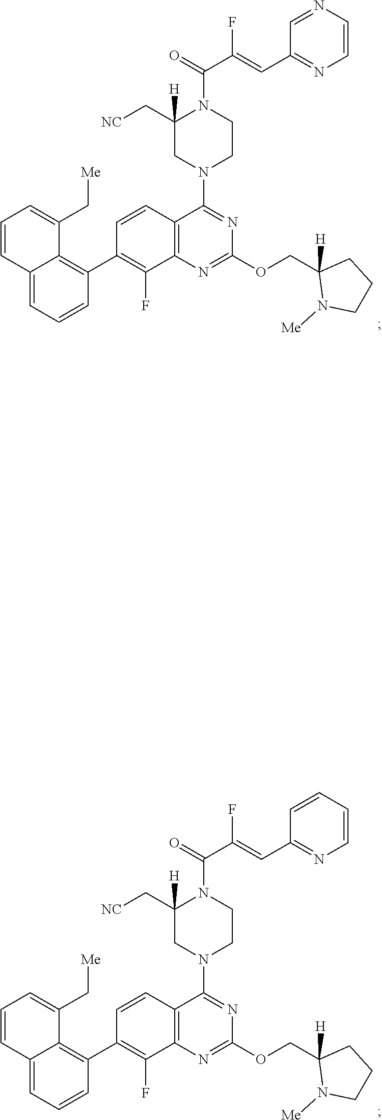 [SMILES: CCc1cccc2cccc(-c3ccc4c(N5CCN(C(=O)/C(F)=C/c6cnccn6)[C@@H](CC#N)C5)nc(OC[C@@H]5CCCN5C)nc4c3F)c12 ; CCc1cccc2cccc(-c3ccc4c(N5CCN(C(=O)/C(F)=C/c6ccccn6)[C@@H](CC#N)C5)nc(OC[C@@H]5CCCN5C)nc4c3F)c12]

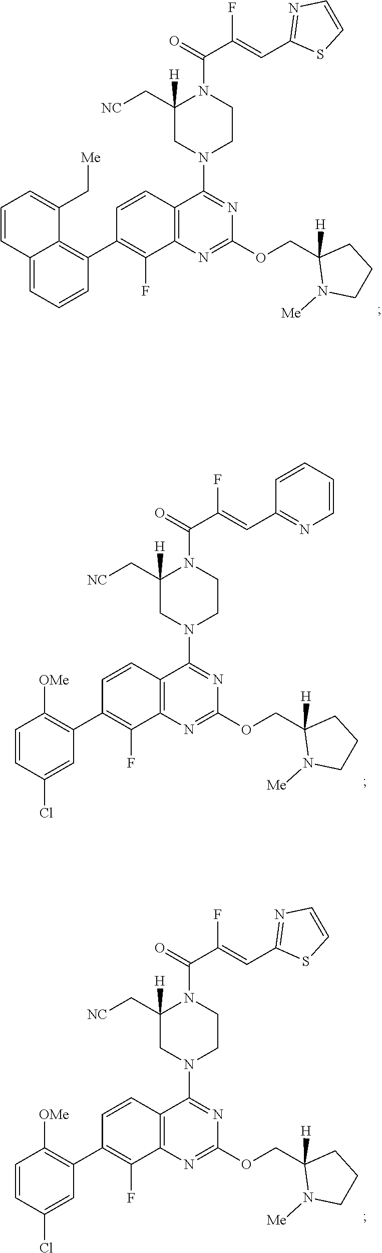 [SMILES: CCc1cccc2cccc(-c3ccc4c(N5CCN(C(=O)/C(F)=C/c6nccs6)[C@@H](CC#N)C5)nc(OC[C@@H]5CCCN5C)nc4c3F)c12 ; COc1ccc(Cl)cc1-c1ccc2c(N3CCN(C(=O)/C(F)=C/c4ccccn4)[C@@H](CC#N)C3)nc(OC[C@@H]3CCCN3C)nc2c1F ; COc1ccc(Cl)cc1-c1ccc2c(N3CCN(C(=O)/C(F)=C/c4nccs4)[C@@H](CC#N)C3)nc(OC[C@@H]3CCCN3C)nc2c1F]

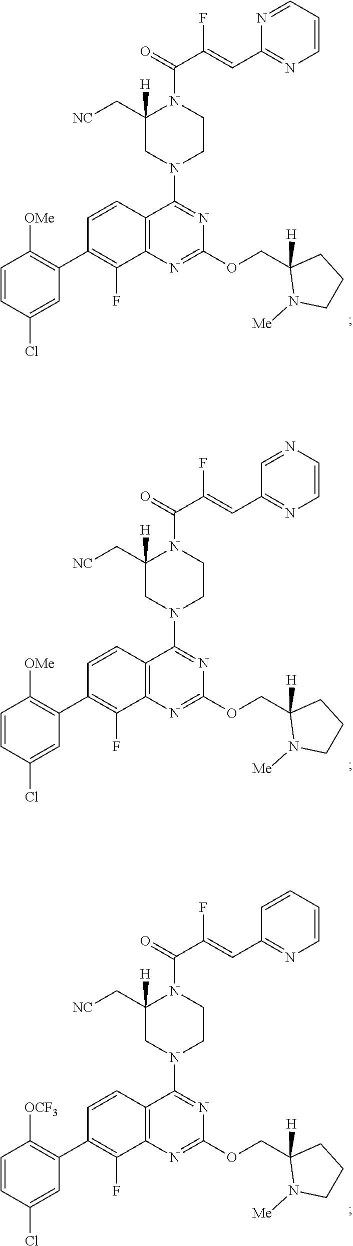 [SMILES: COc1ccc(Cl)cc1-c1ccc2c(N3CCN(C(=O)/C(F)=C/c4ncccn4)[C@@H](CC#N)C3)nc(OC[C@@H]3CCCN3C)nc2c1F ; COc1ccc(Cl)cc1-c1ccc2c(N3CCN(C(=O)/C(F)=C/c4cnccn4)[C@@H](CC#N)C3)nc(OC[C@@H]3CCCN3C)nc2c1F ; CN1CCC[C@H]1COc1nc(N2CCN(C(=O)/C(F)=C/c3ccccn3)[C@@H](CC#N)C2)c2ccc(-c3cc(Cl)ccc3OC(F)(F)F)c(F)c2n1]

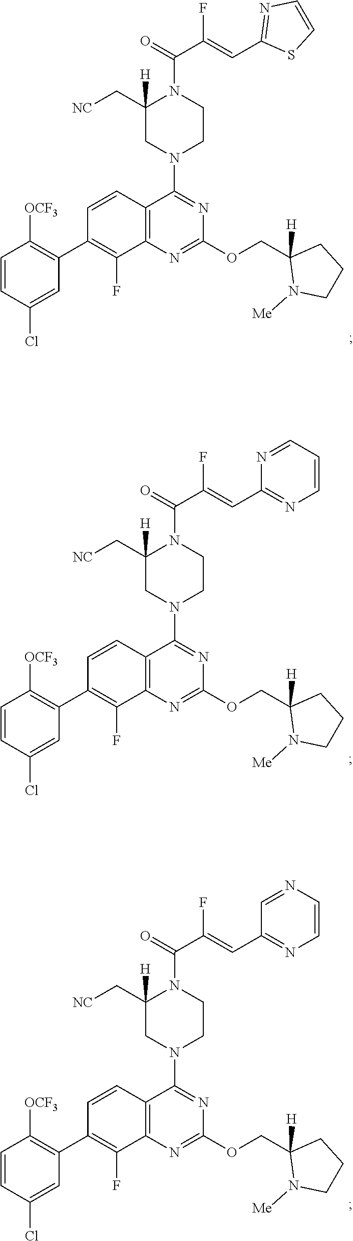 [SMILES: CN1CCC[C@H]1COc1nc(N2CCN(C(=O)/C(F)=C/c3nccs3)[C@@H](CC#N)C2)c2ccc(-c3cc(Cl)ccc3OC(F)(F)F)c(F)c2n1 ; CN1CCC[C@H]1COc1nc(N2CCN(C(=O)/C(F)=C/c3ncccn3)[C@@H](CC#N)C2)c2ccc(-c3cc(Cl)ccc3OC(F)(F)F)c(F)c2n1 ; CN1CCC[C@H]1COc1nc(N2CCN(C(=O)/C(F)=C/c3cnccn3)[C@@H](CC#N)C2)c2ccc(-c3cc(Cl)ccc3OC(F)(F)F)c(F)c2n1]

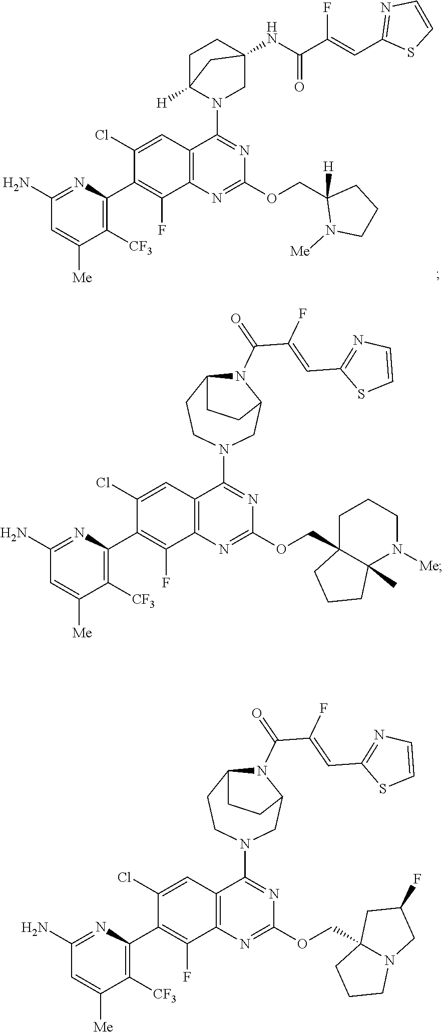

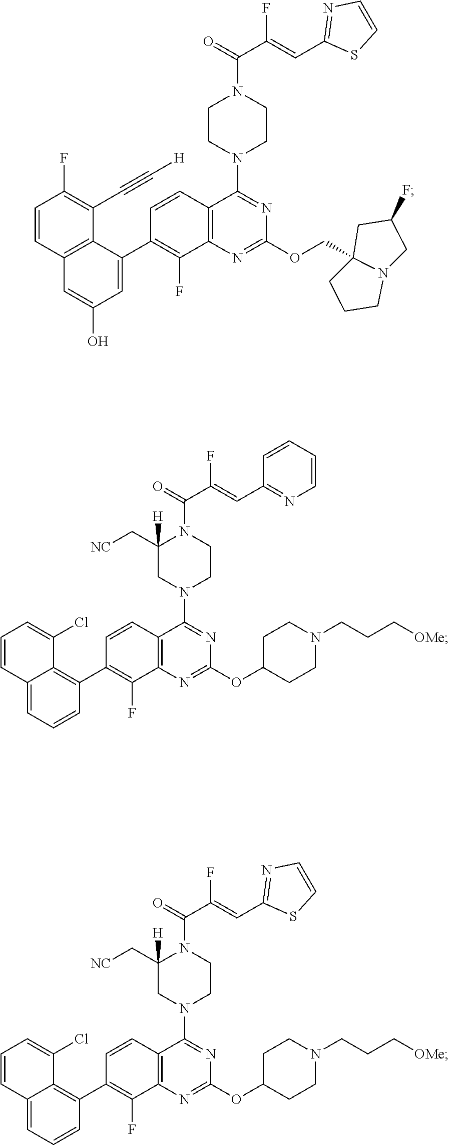 [SMILES: C#Cc1c(F)ccc2cc(O)cc(-c3ccc4c(N5CCN(C(=O)/C(F)=C/c6nccs6)CC5)nc(OC[C@@]56CCCN5C[C@H](F)C6)nc4c3F)c12 ; COCCCN1CCC(Oc2nc(N3CCN(C(=O)/C(F)=C/c4ccccn4)[C@@H](CC#N)C3)c3ccc(-c4cccc5cccc(Cl)c45)c(F)c3n2)CC1 ; COCCCN1CCC(Oc2nc(N3CCN(C(=O)/C(F)=C/c4nccs4)[C@@H](CC#N)C3)c3ccc(-c4cccc5cccc(Cl)c45)c(F)c3n2)CC1]

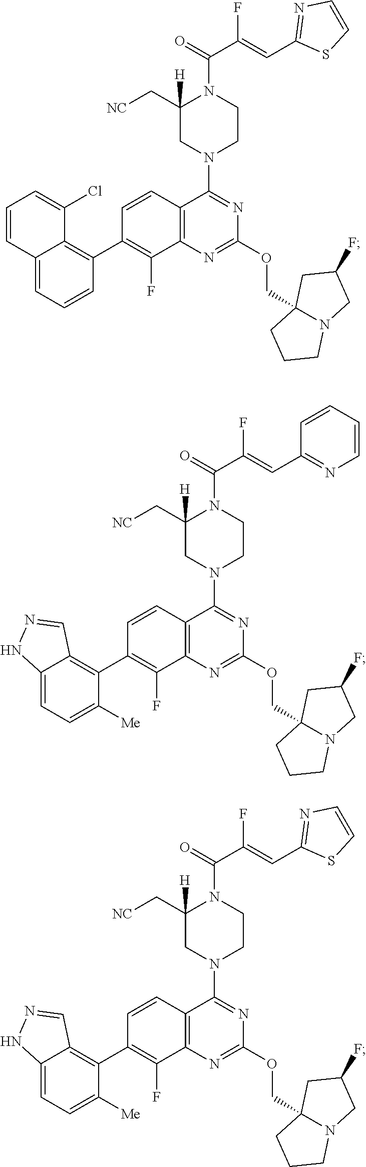

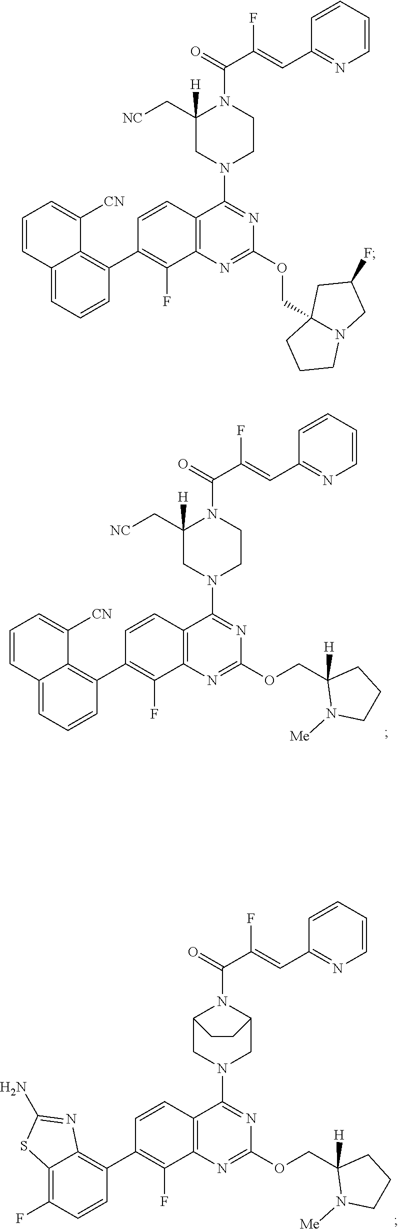 [SMILES: N#CC[C@H]1CN(c2nc(OC[C@@]34CCCN3C[C@H](F)C4)nc3c(F)c(-c4cccc5cccc(C#N)c45)ccc23)CCN1C(=O)/C(F)=C/c1ccccn1 ; CN1CCC[C@H]1COc1nc(N2CCN(C(=O)/C(F)=C/c3ccccn3)[C@@H](CC#N)C2)c2ccc(-c3cccc4cccc(C#N)c34)c(F)c2n1 ; CN1CCC[C@H]1COc1nc(N2CC3CCC(C2)N3C(=O)/C(F)=C/c2ccccn2)c2ccc(-c3ccc(F)c4sc(N)nc34)c(F)c2n1]

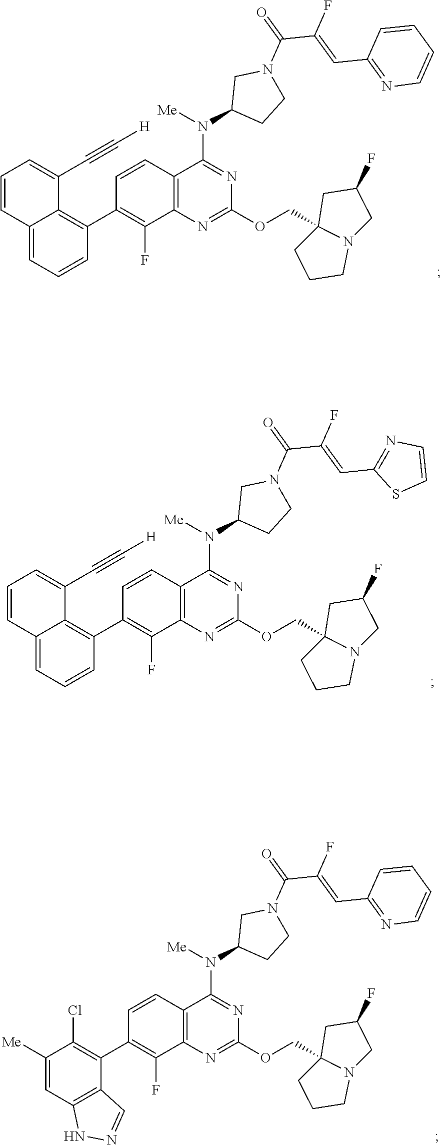 [SMILES: C#Cc1cccc2cccc(-c3ccc4c(N(C)[C@@H]5CCN(C(=O)/C(F)=C/c6ccccn6)C5)nc(OC[C@@]56CCCN5C[C@H](F)C6)nc4c3F)c12 ; C#Cc1cccc2cccc(-c3ccc4c(N(C)[C@@H]5CCN(C(=O)/C(F)=C/c6nccs6)C5)nc(OC[C@@]56CCCN5C[C@H](F)C6)nc4c3F)c12 ; Cc1cc2[nH]ncc2c(-c2ccc3c(N(C)[C@@H]4CCN(C(=O)/C(F)=C/c5ccccn5)C4)nc(OC[C@@]45CCCN4C[C@H](F)C5)nc3c2F)c1Cl]

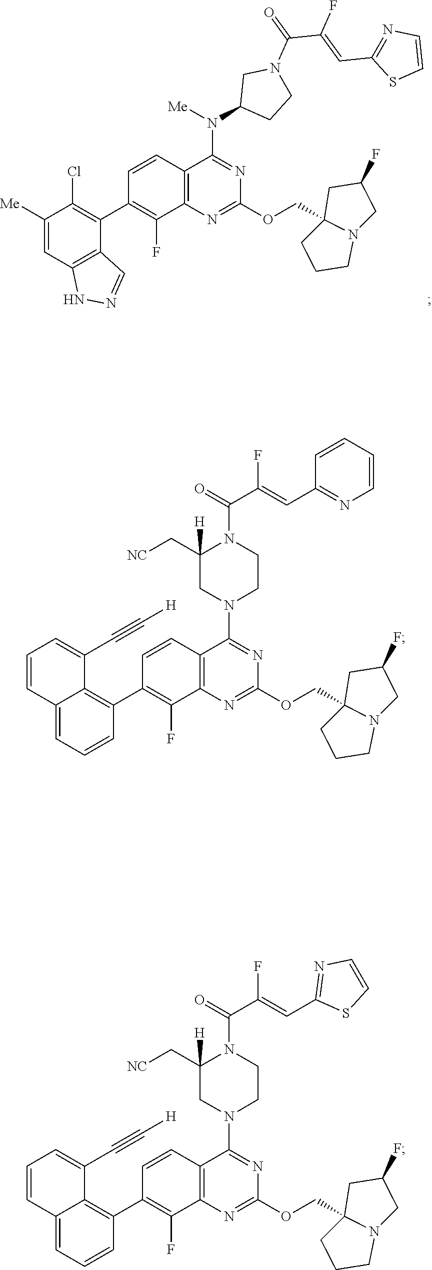 [SMILES: Cc1cc2[nH]ncc2c(-c2ccc3c(N(C)[C@@H]4CCN(C(=O)/C(F)=C/c5nccs5)C4)nc(OC[C@@]45CCCN4C[C@H](F)C5)nc3c2F)c1Cl ; C#Cc1cccc2cccc(-c3ccc4c(N5CCN(C(=O)/C(F)=C/c6ccccn6)[C@@H](CC#N)C5)nc(OC[C@@]56CCCN5C[C@H](F)C6)nc4c3F)c12 ; C#Cc1cccc2cccc(-c3ccc4c(N5CCN(C(=O)/C(F)=C/c6nccs6)[C@@H](CC#N)C5)nc(OC[C@@]56CCCN5C[C@H](F)C6)nc4c3F)c12]

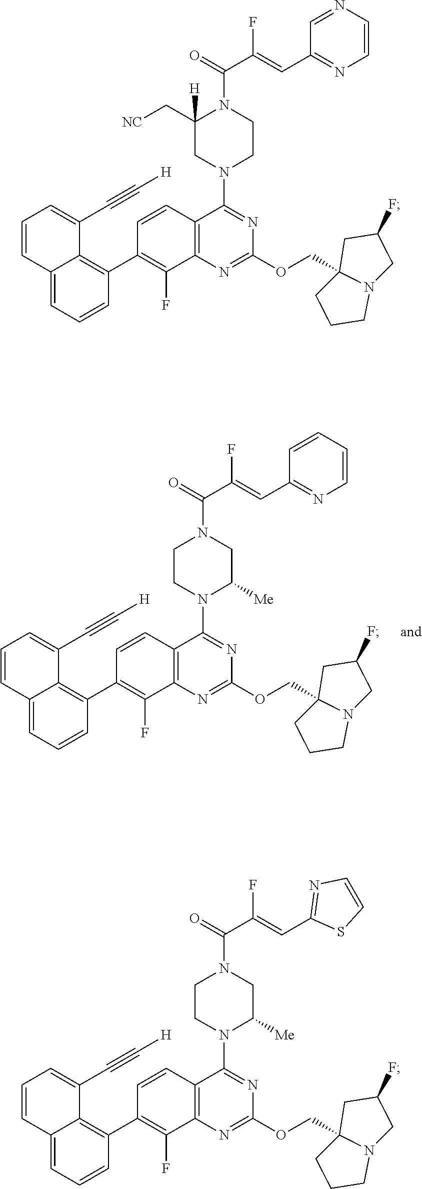 [SMILES: C#Cc1cccc2cccc(-c3ccc4c(N5CCN(C(=O)/C(F)=C/c6cnccn6)[C@@H](CC#N)C5)nc(OC[C@@]56CCCN5C[C@H](F)C6)nc4c3F)c12 ; C#Cc1cccc2cccc(-c3ccc4c(N5CCN(C(=O)/C(F)=C/c6ccccn6)C[C@@H]5C)nc(OC[C@@]56CCCN5C[C@H](F)C6)nc4c3F)c12 ; C#Cc1cccc2cccc(-c3ccc4c(N5CCN(C(=O)/C(F)=C/c6nccs6)C[C@@H]5C)nc(OC[C@@]56CCCN5C[C@H](F)C6)nc4c3F)c12]

or a pharmaceutically acceptable salt thereof.

In some aspects, the present disclosure provides a compound selected from:
(Z)-1-((3S)-4-(6-chloro-7-(8-chloronaphthalen-1-yl)-8-fluoro-2-(((S)-1-methylpyrrolidin-2-yl)methoxy)quinazolin-4-yl)-3-methylpiperazin-1-yl)-2-fluoro-3-(thiazol-2-yl)prop-2-en-1-one;
(Z)-1-((3S)-4-(6-chloro-7-(8-chloronaphthalen-1-yl)-8-fluoro-2-(((S)-1-methylpyrrolidin-2-yl)methoxy)quinazolin-4-yl)-3-methylpiperazin-1-yl)-2-fluoro-3-(pyrimidin-2-yl)prop-2-en-1-one;
(Z)-1-((3S)-4-(6-chloro-7-(8-chloronaphthalen-1-yl)-8-fluoro-2-(((S)-1-methylpyrrolidin-2-yl)methoxy)quinazolin-4-yl)-3-methylpiperazin-1-yl)-2-fluoro-3-(pyridin-2-yl)prop-2-en-1-one;
(Z)-1-((1R,5S)-3-((7M)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one;
(Z)-1-((1R,5S)-3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,8-diazabicyclo[3.2.1]octan-8-yl)-2-fluoro-3-(pyridin-2-yl)prop-2-en-1-one;
(Z)-1-(5-((7M)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-2,5-diazabicyclo[2.2.2]octan-2-yl)-2-fluoro-3-(pyridin-2-yl)prop-2-en-1-one;
(Z)-1-((1R,5S)-3-((7M)-7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octan-8-yl)-2-fluoro-3-(pyridin-2-yl)prop-2-en-1-one;
(Z)-1-((1R,5S)-3-(6-chloro-8-fluoro-7-(3-hydroxynaphthalen-1-yl)-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one;
(Z)-1-(6-((7M)-7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-2,6-diazaspiro[3.4]octan-2-yl)-2-fluoro-3-(thiazol-2-yl)prop-2-en-1-one;
(Z)-1-((1R,5S)-3-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octan-8-yl)-2-fluoro-3-(thiazol-2-yl)prop-2-en-1-one;
(Z)-1-((1R,5S)-3-((7M)-7-(6-amino-4-methyl-3-(trifluoromethyl)pyridin-2-yl)-6-chloro-8-fluoro-2-((1-(piperidin-1-ylmethyl)cyclopropyl)methoxy)quinazolin-4-yl)-3,8-diazabicyclo[3.2.1]octan-8-yl)-2-fluoro-3-(thiazol-2-yl)prop-2-en-1-one;
(Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.1.1]heptan-6-yl)-2-fluoro-3-(thiazol-2-yl)prop-2-en-1-one;
(Z)-N-((1S,3s)-3-(((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)amino)cyclobutyl)-2-fluoro-3-(thiazol-2-yl)acrylamide;
(Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,9-diazabicyclo[3.3.1]nonan-9-yl)-2-fluoro-3-(thiazol-2-yl)prop-2-en-1-one;
(Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.2.0]heptan-6-yl)-2-fluoro-3-(thiazol-2-yl)prop-2-en-1-one (isomer 1);
(Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.2.0]heptan-6-yl)-2-fluoro-3-(thiazol-2-yl)prop-2-en-1-one (isomer 2);
(Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,7-diazabicyclo[4.2.0]octan-7-yl)-2-fluoro-3-(thiazol-2-yl)prop-2-en-1-one;
(Z)-1-((3aR,4S,7R,7aS)-8-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-2H-4,7-epiminoisoindol-2-yl)-2-fluoro-3-(thiazol-2-yl)prop-2-en-1-one;
(Z)-N-((1R,4R)-2-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-2-azabicyclo[2.2.1]heptan-4-yl)-2-fluoro-3-(thiazol-2-yl)acrylamide;
(Z)-1-((2S,5R)-4-((7M)-7-(6-amino-4-methyl-3-(trifluoromethyl)pyridin-2-yl)-6-chloro-8-fluoro-2-((2-fluorotetrahydro-1H-pyrrolizin-7a(5H)-yl)methoxy)quinazolin-4-yl)-2,5-dimethylpiperazin-1-yl)-2-fluoro-3-(thiazol-2-yl)prop-2-en-1-one;
(2Z)-1-{3-[6-chloro-7-(8-ethynyl-7-fluoro-3-hydroxynaphthalen-1-yl)-8-fluoro-2-{[(2S)-1-methylpyrrolidin-2-yl]methoxy}quinazolin-4-yl]-3,8-diazabicyclo[3.2.1]octan-8-yl}-2-fluoro-3-(pyridin-2-yl)prop-2-en-1-one (isomer 1);
(2Z)-1-{3-[6-chloro-7-(8-ethynyl-7-fluoro-3-hydroxynaphthalen-1-yl)-8-fluoro-2-{[(2S)-1-methylpyrrolidin-2-yl]methoxy}quinazolin-4-yl]-3,8-diazabicyclo[3.2.1]octan-8-yl}-2-fluoro-3-(pyridin-2-yl)prop-2-en-1-one (isomer 2);
(Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(thiazol-2-yl)prop-2-en-1-one;
(Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one;
2-((S)-1-((Z)-2-fluoro-3-(thiazol-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile;
2-((S)-1-((Z)-2-fluoro-3-(pyridin-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile;
(Z)-1-(4-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)hexahydropyrrolo[3,2-b]pyrrol-1(2H)-yl)-2-fluoro-3-(thiazol-2-yl)prop-2-en-1-one (isomer 1);
(Z)-1-(4-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)hexahydropyrrolo[3,2-b]pyrrol-1(2H)-yl)-2-fluoro-3-(thiazol-2-yl)prop-2-en-1-one (isomer 2);
(Z)-1-(5-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-1H-pyrrolo[3,2-c]pyridin-1-yl)-2-fluoro-3-(thiazol-2-yl)prop-2-en-1-one (isomer 1);
(Z)-1-(5-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-1H-pyrrolo[3,2-c]pyridin-1-yl)-2-fluoro-3-(thiazol-2-yl)prop-2-en-1-one (isomer 2);
(Z)-1-((1R,5S)-3-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)quinazolin-4-yl)-3,8-diazabicyclo[3.2.1]octan-8-yl)-2-fluoro-3-(pyridin-2-yl)prop-2-en-1-one;
(S,Z)-2-fluoro-1-(4-(8-fluoro-7-(3-hydroxynaphthalen-1-yl)-2-((1-methylpyrrolidin-2-yl)methoxy)quinazolin-4-yl)piperazin-1-yl)-3-(pyridin-2-yl)prop-2-en-1-one;
(S,Z)-2-fluoro-1-(4-(8-fluoro-7-(3-hydroxynaphthalen-1-yl)-2-((1-methylpyrrolidin-2-yl)methoxy)quinazolin-4-yl)piperazin-1-yl)-3-(thiazol-2-yl)prop-2-en-1-one;
(Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(pyrimidin-2-yl)prop-2-en-1-one;
2-((S)-1-((Z)-2-fluoro-3-(pyrimidin-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile;
(S,Z)-2-fluoro-1-(4-(8-fluoro-7-(3-hydroxynaphthalen-1-yl)-2-((1-methylpyrrolidin-2-yl)methoxy)quinazolin-4-yl)piperazin-1-yl)-3-(pyrimidin-2-yl)prop-2-en-1-one;
2-((S)-1-((Z)-2-fluoro-3-(thiazol-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile;
2-((S)-1-((Z)-2-fluoro-3-(6-methylpyridin-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile;
(Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(6-methylpyridin-2-yl)prop-2-en-1-one;
2-((S)-1-((Z)-2-fluoro-3-(6-methylpyridin-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile;
2-((S)-1-((Z)-2-fluoro-3-(pyridin-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile;
2-((S)-1-((Z)-2-fluoro-3-(pyrimidin-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile;
2-((S)-4-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-1-((Z)-2-fluoro-3-(pyridin-2-yl)acryloyl)piperazin-2-yl)acetonitrile;
2-((S)-4-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-1-((Z)-2-fluoro-3-(pyrimidin-2-yl)acryloyl)piperazin-2-yl)acetonitrile;
2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyridin-2-yl)acryloyl)piperazin-2-yl)acetonitrile;
2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyrimidin-2-yl)acryloyl)piperazin-2-yl)acetonitrile;
(2Z)-N-[(1S,4S)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-4-yl]-2-fluoro-3-(pyridin-2-yl)prop-2-enamide;
(2Z)-N-({1-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]piperidin-4-yl}methyl)-2-fluoro-3-(pyridin-2-yl)prop-2-enamide;
(2Z)-N-[(1R,4R,7R)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-7-yl]-2-fluoro-3-(pyridin-2-yl)prop-2-enamide
2-[(2S)-4-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-1-[(2Z)-2-fluoro-3-(thiazol-2-yl)prop-2-enoyl]piperazin-2-yl]acetonitrile
2-[(2S)-4-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-1-[(2Z)-2-fluoro-3-(thiazol-2-yl)prop-2-enoyl]piperazin-2-yl]acetamide
(2Z)-N-[(1S,4S)-2-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-2-azabicyclo[2.2.1]heptan-4-yl]-2-fluoro-3-(thiazol-2-yl)prop-2-enamide;
(2Z)-N-[(1R,4R,7R)-2-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-2-azabicyclo[2.2.1]heptan-7-yl]-2-fluoro-3-(1,3-thiazol-2-yl)prop-2-enamide;
2-((2S)-1-((Z)-2-fluoro-3-(pyridin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile;
2-((2S)-1-((Z)-2-fluoro-3-(thiazol-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile;
2-((2S)-1-((Z)-2-fluoro-3-(pyrimidin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile;
2-((2S)-1-((Z)-2-fluoro-3-(pyrimidin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile;
2-((2S)-1-((Z)-2-fluoro-3-(pyridazin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile;
2-((2S)-1-((Z)-2-fluoro-3-(pyridin-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile;
2-((2S)-1-((Z)-2-fluoro-3-(pyrimidin-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile;
2-((2S)-1-((Z)-2-fluoro-3-(thiazol-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile;
2-((2S)-1-((Z)-2-fluoro-3-(pyridazin-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile;
2-((S)-4-(7-(8-ethylnaphthalen-1-yl)-8-fluoro-2-(((S)-1-methylpyrrolidin-2-yl)methoxy)quinazolin-4-yl)-1-((Z)-2-fluoro-3-(pyrazin-2-yl)acryloyl)piperazin-2-yl)acetonitrile;
2-((S)-4-(7-(8-ethylnaphthalen-1-yl)-8-fluoro-2-(((S)-1-methylpyrrolidin-2-yl)methoxy)quinazolin-4-yl)-1-((Z)-2-fluoro-3-(pyridin-2-yl)acryloyl)piperazin-2-yl)acetonitrile;
2-((S)-4-(7-(8-ethylnaphthalen-1-yl)-8-fluoro-2-(((S)-1-methylpyrrolidin-2-yl)methoxy)quinazolin-4-yl)-1-((Z)-2-fluoro-3-(thiazol-2-yl)acryloyl)piperazin-2-yl)acetonitrile;
2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(pyridin-2-yl)acryloyl)piperazin-2-yl)acetonitrile;
2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(thiazol-2-yl)acryloyl)piperazin-2-yl)acetonitrile;
2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(pyrimidin-2-yl)acryloyl)piperazin-2-yl)acetonitrile;
2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(piperazin-2-yl)acryloyl)piperazin-2-yl)acetonitrile;
2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(pyridin-2-yl)acryloyl)piperazin-2-yl)acetonitrile;
2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(thiazol-2-yl)acryloyl)piperazin-2-yl)acetonitrile;
2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(pyrimidin-2-yl)acryloyl)piperazin-2-yl)acetonitrile;
2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(pyrazin-2-yl)acryloyl)piperazin-2-yl)acetonitrile;
(Z)-N-((1S,4S)-2-(7M-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-2-azabicyclo[2.2.1]heptan-4-yl)-2-fluoro-3-(thiazol-2-yl)acrylamide;
(Z)-1-((1R,6S)-3-(7M-(6-amino-4-methyl-3-(trifluoromethyl)pyridin-2-yl)-6-chloro-8-fluoro-2-(((4aS,7aR)-1-methyloctahydro-4aH-cyclopenta[b]pyridin-4a-yl)methoxy)quinazolin-4-yl)-3,9-diazabicyclo[4.2.1]nonan-9-yl)-2-fluoro-3-(thiazol-2-yl)prop-2-en-1-one;
(Z)-1-((1R,6S)-3-(7M-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,9-diazabicyclo[4.2.1]nonan-9-yl)-2-fluoro-3-(thiazol-2-yl)prop-2-en-1-one;
(Z)-1-(4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)piperazin-1-yl)-2-fluoro-3-(thiazol-2-yl)prop-2-en-1-one;
(S,Z)-2-(4-(7-(8-chloronaphthalen-1-yl)-8-fluoro-2-((1-(3-methoxypropyl)piperidin-4-yl)oxy)quinazolin-4-yl)-1-(2-fluoro-3-(pyridin-2-yl)acryloyl)piperazin-2-yl)acetonitrile;
(S,Z)-2-(4-(7-(8-chloronaphthalen-1-yl)-8-fluoro-2-((1-(3-methoxypropyl)piperidin-4-yl)oxy)quinazolin-4-yl)-1-(2-fluoro-3-(thiazol-2-yl)acryloyl)piperazin-2-yl)acetonitrile;
2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(thiazol-2-yl)acryloyl)piperazin-2-yl)acetonitrile;
2-((2S)-4-(8-fluoro-2-(((2R,7aS)-2-fluorotetrahydro-1H-pyrrolizin-7a(5H)-yl)methoxy)-7-(5-methyl-1H-indazol-4-yl)quinazolin-4-yl)-1-((Z)-2-fluoro-3-(pyridin-2-yl)acryloyl)piperazin-2-yl)acetonitrile;
2-((2S)-4-(8-fluoro-2-(((2R,7aS)-2-fluorotetrahydro-1H-pyrrolizin-7a(5H)-yl)methoxy)-7-(5-methyl-1H-indazol-4-yl)quinazolin-4-yl)-1-((Z)-2-fluoro-3-(thiazol-2-yl)acryloyl)piperazin-2-yl)acetonitrile;
8-(4-((S)-3-(cyanomethyl)-4-((Z)-2-fluoro-3-(pyridin-2-yl)acryloyl)piperazin-1-yl)-8-fluoro-2-(((2R,7aS)-2-fluorotetrahydro-1H-pyrrolizin-7a(5H)-yl)methoxy)quinazolin-7-yl)-1-naphthonitrile;
8-(4-((S)-3-(cyanomethyl)-4-((Z)-2-fluoro-3-(pyridin-2-yl)acryloyl)piperazin-1-yl)-8-fluoro-2-(((S)-1-methylpyrrolidin-2-yl)methoxy)quinazolin-7-yl)-1-naphthonitrile;
(Z)-1-((1R,5S)-3-(7-(2-amino-7-fluorobenzo[d]thiazol-4-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one;
(Z)-1-((R)-3-((7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(pyridin-2-yl)prop-2-en-1-one;
(Z)-1-((R)-3-((7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(thiazol-2-yl)prop-2-en-1-one;
(Z)-1-((3R)-3-((7-(5-chloro-6-methyl-1H-indazol-4-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(pyridin-2-yl)prop-2-en-1-one;
(Z)-1-((3R)-3-((7-(5-chloro-6-methyl-1H-indazol-4-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(thiazol-2-yl)prop-2-en-1-one;
2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyridin-2-yl)acryloyl)piperazin-2-yl)acetonitrile;
2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(thiazol-2-yl)acryloyl)piperazin-2-yl)acetonitrile;
2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyrazin-2-yl)acryloyl)piperazin-2-yl)acetonitrile;
(Z)-1-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-3-methylpiperazin-1-yl)-2-fluoro-3-(pyridin-2-yl)prop-2-en-1-one; and
(Z)-1-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-3-methylpiperazin-1-yl)-2-fluoro-3-(thiazol-2-yl)prop-2-en-1-one;
   or a pharmaceutically acceptable salt thereof.

In some aspects, the present disclosure provides an atropisomer of a compound of any of the prior aspects. In certain aspects, the compound is a stable atropisomer as described herein.

In some aspects, the present disclosure provides a pharmaceutical composition comprising a compound of any of the prior aspects, or a pharmaceutically acceptable salt thereof, and one or more pharmaceutically acceptable excipients.

In some aspects, the present disclosure provides an oral dosage form comprising a compound of any of the prior aspects, or a pharmaceutically acceptable salt thereof, and one or more pharmaceutically acceptable excipients.

In some aspects, the present disclosure provides a method of treating cancer expressing KRAS G12C, G12D and/or G12V mutation in a subject in need thereof, the method comprising administering to the subject a compound of any of the prior aspects, or a pharmaceutically acceptable salt thereof.

In some aspects, the present disclosure provides a method of treating cancer expressing KRAS G12C mutation in a subject in need thereof, the method comprising administering to the subject a compound of any of the prior aspects, or a pharmaceutically acceptable salt thereof.

In some aspects, the present disclosure provides a method for treating a cancer susceptible to KRAS G12C inhibition in a subject in need thereof, the method comprising administering to the subject a compound of any of the prior aspects, or a pharmaceutically acceptable salt thereof.

In some aspects, the present disclosure provides a method for treating a cancer in a subject in need thereof, the method comprising administering to the subject a compound of any of the prior aspects, or a pharmaceutically acceptable salt thereof, wherein the cancer is lung cancer, colorectal cancer, pancreatic cancer, breast cancer, bladder cancer, cervical cancer, ovarian cancer, gastric cancer or cancer of the uterus.

In some aspects, the present disclosure provides a method for treating a cancer in a subject in need thereof, the method comprising administering to the subject a compound of any of the prior aspects, or a pharmaceutically acceptable salt thereof, wherein the cancer is non-small cell lung cancer.

In some aspects of the method, the compound is an atropisomer of a compound of any of the prior aspects. In certain aspects, the compound is a stable atropisomer as described herein.

In another aspect, the present disclosure provides a method for inhibiting KRAS G12C activity in a in a cell, comprising contacting the cell with a therapeutically effective amount of a compound of formula (I), or a pharmaceutically acceptable salt thereof, or a pharmaceutical composition thereof as defined herein. In one aspect, the contacting is in vitro. In one aspect, the contacting is in vivo.

In some aspects, the present disclosure provides a method of inhibiting cell proliferation, in vitro or in vivo, the method comprising contacting a cell with a therapeutically effective amount of a compound of formula (I), or a pharmaceutically acceptable salt thereof, or a pharmaceutical composition thereof as defined herein.

In another aspect, the present disclosure provides a compound of formula (I), or a pharmaceutically acceptable salt thereof, for use in the inhibition of KRAS G12C.

In another aspect, the present disclosure provides a compound of formula (I), or a pharmaceutically acceptable salt thereof, or a pharmaceutical composition thereof as defined herein, for use in the treatment of a KRAS G12C-associated disease or disorder.

In another aspect, the present disclosure provides a use of a compound of formula (I), or a pharmaceutically acceptable salt thereof, as defined herein in the manufacture of a medicament for the treatment of cancer. In some aspects, the cancer is lung cancer. In some aspects, the cancer is non-small cell lung cancer.

In another aspect, the present disclosure provides the use of a compound of formula (I), or a pharmaceutically acceptable salt thereof, as defined herein in the manufacture of a medicament for the inhibition of activity of KRAS G12C.

In another aspect, the present disclosure provides the use of a compound of formula (I), or a pharmaceutically acceptable salt thereof, as defined herein, in the manufacture of a medicament for the treatment of a KRAS G12C-associated disease or disorder.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

The term “C₂-C₄alkenyl”, as used herein, refers to a group derived from a straight or branched chain hydrocarbon containing from two to four carbon atoms and one double bond.

The term “C₁-C₃alkoxy”, as used herein, refers to a C₁-C₃alkyl group attached to the parent molecular moiety through an oxygen atom.

The term “C₁-C₆alkoxy”, as used herein, refers to a C₁-C₆alkyl group attached to the parent molecular moiety through an oxygen atom.

The term “C₁-C₃alkoxyC₁-C₃alkyl,” as used herein, refers to a C₁-C₃alkoxy group attached to the parent molecular moiety through a C₁-C₃alkyl group.

The term “C₁-C₆alkoxyC₁-C₆alkyl,” as used herein, refers to a C₁-C₆alkoxy group attached to the parent molecular moiety through a C₁-C₆alkyl group.

The term “C₁-C₃alkoxycarbonyl”, as used herein, refers to a C₁-C₃alkoxy group attached to the parent molecular moiety through a carbonyl group.

The term “C₁-C₃alkyl”, as used herein, refers to a group derived from a straight or branched chain saturated hydrocarbon containing from one to three carbon atoms.

The term “C₁-C₆alkyl”, as used herein, refers to a group derived from a straight or branched chain saturated hydrocarbon containing from one to six carbon atoms.

The term “(C₁-C₆alkyl)amino,” as used herein, refers to R—NH, wherein R is a C₁-C₆alkyl group.

The term “(C₁-C₆alkyl)aminoC₁-C₃alkyl,” as used herein, refers to a (C₁-C₆alkyl)amino group attached to the parent molecular moiety through a C₁-C₃alkyl group.

The term “C₁-C₃alkylcarbonyl”, as used herein, refers to a C₁-C₃alkyl group attached to the parent molecular moiety through a carbonyl group.

The term “C₂-C₄alkynyl”, as used herein, refers to a group derived from a straight or branched chain hydrocarbon containing from two to four carbon atoms and one triple bond.

The term “amido,” as used herein, refers to NH₂C(O)—.

The term “amidoC₁-C₃alkyl,” as used herein, refers to an amido group attached to the parent molecular group through a C₁-C₃alkyl group.

The term “amino,” as used herein, refers to —NH₂.

The term “aminoC₁-C₃alkyl,” as used herein, refers to an amino group attached to the parent molecular moiety through a C₁-C₃alkyl group.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “arylC₁-C₆alkyl,” as used herein refers to an aryl group attached to the parent molecular moiety through a C₁-C₆alkyl group.

The term “carboxy,” as used herein, refers to —CO₂H.

The term “carboxyC₁-C₆alkyl,” as used herein, refers to a C₁-C₆alkyl group substituted with one, two, or three carboxy groups.

The term “cyano,” as used herein, refers to —CN.

The term “cyanoC₁-C₃alkyl,” as used herein, refers to a C₁-C₃alkyl group substituted with one, two, or three cyano groups.

The term “C₃-C₄cycloalkyl”, as used herein, refers to a saturated monocyclic hydrocarbon ring system having three or four carbon atoms and zero heteroatoms.

The term “C₃-C₆cycloalkyl”, as used herein, refers to a saturated monocyclic hydrocarbon ring system having three to six carbon atoms and zero heteroatoms.

The term “C₃-C₆cycloalkylC₁-C₆alkyl”, as used herein, refers to a C₃-C₆cycloalkyl attached to the parent molecular moiety through a C₁-C₆alkyl group.

The term “di(C₁-C₆alkyl)amino”, as used herein, refers to —NR^{z}R^{z′}, wherein R^{z} and R^{z′} are the same or different C₁-C₆alkyl groups.

The term “di(C₁-C₃alkyl)aminoC₂-C₆alkyl,” as used herein, refers to —(C₂-C₆alkyl)NR^{z}R^{z′}, wherein R^{z} and R^{z′} are the same or different C₁-C₆alkyl groups.

The terms “halo” and “halogen”, as used herein, refer to F, Cl, Br, or I.

The term “haloC₁-C₃alkoxy”, as used herein, refers to a C₁-C₃alkoxy group substituted with one, two, or three halogen atoms.

The term “haloC₁-C₃alkyl”, as used herein, refers to a C₁-C₃alkyl group substituted with one, two, or three halogen atoms.

The term “haloC₁-C₆alkyl”, as used herein, refers to a C₁-C₆alkyl group substituted with one to six halogen atoms.

The term “heteroaryl,” as used herein, refers to an aromatic five- or six-membered ring where at least one atom is selected from N, O, and S, and the remaining atoms are carbon. The term “heteroaryl” also includes bicyclic systems where a heteroaryl ring is fused to a four- to six-membered aromatic or non-aromatic ring containing zero, one, or two additional heteroatoms selected from N, O, and S; and tricyclic systems where a bicyclic system is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, alloxazine, benzo[1,2-d:4,5-d′]bisthiazole, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, purine, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

The term “heteroarylC₁-C₆alkyl, as used herein, refers to a heteroaryl group attached to the parent molecular moiety through a C₁-C₆alkyl group.

The term “heterocyclyl”, as used herein, refers to a five-, six-, seven-, eight-, nine-, ten-, eleven-, or twelve-membered saturated or partially unsaturated ring containing one, two, or three heteroatoms independently selected from nitrogen, oxygen, and sulfur. The term “heterocyclyl” also includes groups in which the heterocyclyl ring is fused to one, two, or three four- to six-membered aromatic or non-aromatic carbocyclic rings or monocyclic heterocyclyl groups. The term “heterocyclyl” also includes monocyclic or polycyclic heterocyclyl group as described above which are further substituted with one or more spirocyclic groups that are attached to the heterocyclyl group through a spiro carbon. Examples of heterocyclyl groups include, but are not limited to, dihydro-1′H,3′H,5′H-dispiro[cyclopropane-1,2′-pyrrolizine-6′,1″-cyclopropane], hexahydro-2H-1,4-dioxa-2a1-azacyclopenta[cd]pentalenyl, hexahydropyrrolizinyl, indolinyl, morpholinyl, octahydroindolizinyl, octahydroquinolizinyl, piperazinyl, piperidinyl, pyrrolidinyl, tetrahydrofuranyl, tetrahydropyranyl, and thiomorpholinyl.

The term “heterocyclylC₁-C₆alkyl,” as used herein, refers to a heterocyclyl group attached to the parent molecular moiety through a C₁-C₆alkyl group.

The term “hydroxy,” as used herein, refers to —OH.

The term “hydroxyC₁-C₃alkyl,” as used herein, refers to a hydroxy group attached to the parent molecular moiety through a C₁-C₃alkyl group.

The term “hydroxyC₁-C₆alkyl,” as used herein, refers to a hydroxy group attached to the parent molecular moiety through a C₁-C₆alkyl group.

The term “NR^{a}R^{b}—C(O),” as used herein, refers to an NR^{a}R^{b} group attached to the parent molecular moiety through a carbonyl group.

The term “NR^{a}R^{b}—C(O)—C₁-C₆alkyl,” as used herein, refers to an NR^{a}R^{b}—C(O)— group attached to the parent molecular moiety through a C₁-C₆alkyl group.

The term “NR^{a}R^{b}C₁-C₆alkyl, as used herein, refers to an NR^{a}R^{b} group attached to the parent molecular moiety through a C₁-C₆alkyl group.

The term “nitro,” as used herein, refers to —NO₂.

The term “oxo,” as used herein, refers to ═O.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a compound described herein can be prepared using a radioactive isotope and the resulting radiolabeled compound can be used to develop a binding assay or for metabolism studies. Alternatively, and for the same purpose, a compound described herein can be converted to a radiolabeled form by catalytic tritiation using methods known to those skilled in the art.

Certain compounds of the present disclosure exist as stereoisomers. It should be understood that when stereochemistry is not specified, the present disclosure encompasses all stereochemical isomeric forms, or mixtures thereof, which possess the ability inhibit KRAS G12C. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure exist as atropisomers. The term “atropisomers” refers to conformational stereoisomers which occur when rotation about a single bond in the molecule is prevented, or greatly slowed, as a result of steric interactions with other parts of the molecule and the substituents at both ends of the single bond are asymmetrical (i.e., optical activity arises without requiring an asymmetric carbon center or stereocenter). Where the rotational barrier about the single bond is high enough, and interconversion between conformations is slow enough, separation and isolation of the isomeric species may be permitted. Atropisomers are enantiomers (or epimers) without a single asymmetric atom.

The atropisomers can be considered stable if the barrier to interconversion is high enough to permit the atropisomers to undergo little or no interconversion at room temperature for at least a week. In some aspects the atropisomers undergo little or no interconversion at room temperature for at least a year. In some aspects, an atropisomeric compound of the disclosure does not undergo more than about 5% interconversion to its opposite atropisomer at room temperature during one week when the atropisomeric compound is in substantially pure form, which is generally a solid state. In some aspects, an atropisomeric compound of the disclosure does not undergo more than about 5% interconversion to its opposite atropisomer at room temperature (approximately 25° C.) during one year. In some aspects, the atropisomeric compounds of the disclosure are stable enough to undergo no more than about 5% interconversion in an aqueous pharmaceutical formulation held at 0° C. for at least one week. The present chemical entities, pharmaceutical compositions and methods are meant to include all such possible atropisomers, including racemic mixtures, diastereomeric mixtures, epimeric mixtures, optically pure forms of single atropisomers, and intermediate mixtures.

The energy barrier to thermal racemization of atropisomers may be determined by the steric hindrance to free rotation of one or more bonds forming a chiral axis. Certain biaryl compounds exhibit atropisomerism where rotation around an interannular bond lacking C2 symmetry is restricted. The free energy barrier for isomerization (enantiomerization) is a measure of the stability of the interannular bond with respect to rotation. Optical and thermal excitation can promote racemization of such isomers, dependent on electronic and steric factors.

Ortho-substituted biaryl compounds may exhibit this type of conformational, rotational isomerism. Such biaryls are enantiomeric, chiral atropisomers where the sp²-sp² carbon-carbon, interannular bond between the aryl rings has a sufficiently high energy barrier to prevent free rotation, and where substituents W¹≠W² and W³≠W⁴ render the molecule asymmetric.

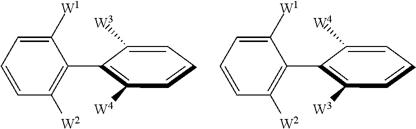

The steric interaction between W¹:W³, W¹:W⁴, and/or W²:W⁴, W²:W³ is large enough to make the planar conformation an energy maximum. Two non-planar, axially chiral enantiomers then exist as atropisomers when their interconversion is slow enough such that they can be isolated free of each other. Bold lines and dashed lines in the figures shown above indicate those moieties, or portions of the molecule, which are sterically restricted due to a rotational energy barrier. Balded moieties exist orthogonally above the plane of the page, and dashed moieties exist orthogonally below the plane of the page. The ‘flat’ part of the molecule (the left ring in each of the two depicted biaryls) is in the plane of the page.

The pharmaceutical compositions of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents, as described herein.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

The pharmaceutical compositions of the present disclosure can be administered via one or more routes of administration using one or more of a variety of methods known in the art. As will be appreciated by the skilled artisan, the route and/or mode of administration will vary depending upon the desired results. In some aspects, the routes of administration for compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are reduced pressure drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, and injectable organic esters. Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution or as a liquid with ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one nontoxic pharmaceutically acceptable excipient suitable for the manufacture of tablets.

An aqueous suspension can be prepared, for example, by admixing at least one compound of Formula (I) and/or at least one pharmaceutically acceptable salt thereof with at least one excipient suitable for the manufacture of an aqueous suspension, including, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one aspect, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain aspects, the compounds of the present disclosure can be administered parenterally, i.e., by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and/or infusion.

In some aspects, the compounds of the present disclosure can be administered orally, i.e, via a gelatin capsule, tablet, hard or soft capsule, or a liquid capsule.

### Use of KRAS Inhibitors/Methods of Treating

Administration of a therapeutic agent described herein may include administration of a therapeutically effective amount of therapeutic agent. The term “therapeutically effective amount” as used herein refers, without limitation, to an amount of a therapeutic agent to treat a condition treatable by administration of a composition comprising the KRAS inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration.

For administration of the compounds described herein, the dosage ranges from about 0.0001 to 100 mg/kg, and more usually 0.01 to 40 mg/kg, of the host body weight. An exemplary treatment regime entails administration once per day, bi-weekly, tri-weekly, weekly, once every two weeks, once every three weeks, once every four weeks, once a month, once every 3 months or once every three to 6 months.

The disclosed compounds strongly inhibit anchorage-independent cell growth and therefore have the potential to inhibit tumor metastasis. Accordingly, in another aspect the disclosure provides a method for inhibiting tumor metastasis, the method comprising administering an effective amount a pharmaceutical composition of comprising any of the compounds disclosed herein and a pharmaceutically acceptable carrier to a subject in need thereof.

Ras mutations including but not limited to KRAS mutations have also been identified in hematological malignancies (e.g., cancers that affect blood, bone marrow and/or lymph nodes). Accordingly, certain aspects are directed to administration of a disclosed compounds (e.g., in the form of a pharmaceutical composition) to a patient in need of treatment of a hematological malignancy. Such malignancies include, but are not limited to, leukemias and lymphomas. For example, the presently disclosed compounds can be used for treatment of diseases such as Acute lymphoblastic leukemia (ALL), Acute myelogenous leukemia (AML), Chronic lymphocytic leukemia (CLL), small lymphocytic lymphoma (SLL), Chronic myelogenous leukemia (CML), Acute monocytic leukemia (AMoL) and/or other leukemias. In other aspects, the compounds are useful for treatment of lymphomas such as all subtypes of Hodgkins lymphoma or non-Hodgkins lymphoma.

Determining whether a tumor or cancer comprises a KRAS mutation can be undertaken by assessing the nucleotide sequence encoding the KRAS protein, by assessing the amino acid sequence of KRAS protein, or by assessing the characteristics of a putative KRAS mutant protein. The sequence of wild-type human KRAS proteins is known in the art.

Methods for detecting a KRAS mutation are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some aspects, samples are evaluated for KRAS mutations including by real-time PCR. In real-time PCR, fluorescent probes specific for the KRAS mutation are used. When a mutation is present, the probe binds and fluorescence is detected. In some aspects, the KRAS mutation is identified using a direct sequencing method of specific regions (e.g., exon 2 and/or exon 3) in the KRAS gene, for example. This technique will identify all possible mutations in the region sequenced.

Methods for detecting a mutation in a KRAS protein are known by those of skill in the art. These methods include, but are not limited to, detection of a KRAS mutant using a binding agent (e.g., an antibody) specific for the mutant protein, protein electrophoresis and Western blotting, and direct peptide sequencing.

Methods for determining whether a tumor or cancer comprises a KRAS mutation can use a variety of samples. In some aspects, the sample is taken from a subject having a tumor or cancer. In some aspects, the sample is taken from a subject having a cancer or tumor. In some aspects, the sample is a fresh tumor/cancer sample. In some aspects, the sample is a frozen tumor/cancer sample. In some aspects, the sample is a formalin-fixed paraffin-embedded sample. In some aspects, the sample is processed to a cell lysate. In some aspects, the sample is processed to DNA or RNA. The disclosure also relates to a method of treating a hyperproliferative disorder in a mammal that comprises administering to said mammal a therapeutically effective amount of a compound of the present disclosure, or a pharmaceutically acceptable salt, ester, prodrug, solvate, hydrate or derivative thereof. In some aspects, said method relates to the treatment of cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CVL), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, isletcell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some aspects, said method relates to the treatment of a non-cancerous hyperproliferative disorder such as benign hyperplasia of the skin (e.g., psoriasis), restenosis, or prostate (e.g., benign prostatic hypertrophy (BPH)).

In certain aspects, the disclosure relates to methods for treatment of lung cancers, the methods comprise administering an effective amount of any of the above-described compound (or a pharmaceutical composition comprising the same) to a subject in need thereof. In certain aspects the lung cancer is a non-small cell lung carcinoma (NSCLC), for example adenocarcinoma, squamous-cell lung carcinoma or large-cell lung carcinoma. In other aspects, the lung cancer is a small cell lung carcinoma. Other lung cancers treatable with the disclosed compounds include, but are not limited to, glandular tumors, carcinoid tumors and undifferentiated carcinomas. Subjects that can be treated with compounds of the disclosure, or pharmaceutically acceptable salt, ester, prodrug, solvate, tautomer, hydrate or derivative of said compounds, according to the methods of this disclosure include, for example, subjects that have been diagnosed as having acute myeloid leukemia, acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germcell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasalcavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some aspects subjects that are treated with the compounds of the disclosure include subjects that have been diagnosed as having a non-cancerous hyperproliferative disorder such as benign hyperplasia of the skin (e. g., psoriasis), restenosis, or prostate (e. g., benign pro static hypertrophy (BPH)). The disclosure further provides methods of modulating a mutant KRAS protein activity by contacting the protein with an effective amount of a compound of the disclosure. Modulation can be inhibiting or activating protein activity. In some aspects, the disclosure provides methods of inhibiting protein activity by contacting the mutant KRAS protein with an effective amount of a compound of the disclosure in solution. In some aspects, the disclosure provides methods of inhibiting the mutant KRAS protein activity by contacting a cell, tissue, organ that express the protein of interest. In some aspects, the disclosure provides methods of inhibiting protein activity in a subject including but not limited to rodents and mammal (e.g., human) by administering into the subject an effective amount of a compound of the disclosure. In some aspects, the percentage modulation exceeds 25%, 30%, 40%, 50%, 60%, 70%, 80%, or 90%. In some aspects, the percentage of inhibiting exceeds 25%, 30%, 40%, 50%, 60%, 70%, 80%, or 90%. In some aspects, the disclosure provides methods of inhibiting KRAS activity in a cell by contacting said cell with an amount of a compound of the disclosure sufficient to inhibit the activity of a KRAS mutant in said cell. In some aspects, the disclosure provides methods of inhibiting mutant KRAS in a tissue by contacting said tissue with an amount of a compound of the disclosure sufficient to inhibit the activity of mutant KRAS in said tissue. In some aspects, the disclosure provides methods of inhibiting KRAS in an organism by contacting said organism with an amount of a compound of the disclosure sufficient to inhibit the activity of KRAS in said organism. In some aspects, the disclosure provides methods of inhibiting KRAS activity in an animal by contacting said animal with an amount of a compound of the disclosure sufficient to inhibit the activity of KRAS in said animal. In some aspects, the disclosure provides methods of inhibiting KRAS including in a mammal by contacting said mammal with an amount of a compound of the disclosure sufficient to inhibit the activity of KRAS in said mammal. In some aspects, the disclosure provides methods of inhibiting KRAS activity in a human by contacting said human with an amount of a compound of the disclosure sufficient to inhibit the activity of KRAS in said human. The present disclosure provides methods of treating a disease mediated by KRAS activity in a subject in need of such treatment. The present disclosure also provides methods for combination therapies in which an agent known to modulate other pathways, or other components of the same pathway, or even overlapping sets of target enzymes are used in combination with a compound of the present disclosure, or a pharmaceutically acceptable salt, ester, prodrug, solvate, tautomer, hydrate or derivative thereof. In one aspect, such therapy includes but is not limited to the combination of one or more compounds of the disclosure with chemotherapeutic agents, therapeutic antibodies, and radiation treatment.

Many chemotherapeutics are presently known in the art and can be used in combination with the compounds of the disclosure. In some aspects, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

The compounds described herein can be used in combination with the agents disclosed herein or other suitable agents, depending on the condition being treated. Hence, in some aspects, the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some aspects of the separate administration protocol, a compound of the disclosure and any of the agents described above are administered a few minutes apart, or a few hours apart, or a few days apart.

The compounds can be made by methods known in the art including those described below and including variations within the skill of the art. Some reagents and intermediates are known in the art. Other reagents and intermediates can be made by methods known in the art using readily available materials. Any variables (e.g., numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

### Synthesis

The aspects described herein are further defined in the following Examples. It should be understood that the Examples are given by way of illustration only. From the above discussion and the Examples, one skilled in the art can ascertain the essential characteristics of the aspects described herein, and without departing from the spirit and scope thereof, can make various changes and modifications to them to adapt to various uses and conditions. As a result, the aspects described herein are not limited by the illustrative examples set forth herein below, but rather are defined by the claims appended hereto.

### Abbreviations

The following abbreviations are used in the example section below and elsewhere herein: AA for ammonium acetate; ACN for acetonitrile; BOC or Boc for tert-butoxycarbonyl; Cbz for carbobenzyloxy; DCM for dichloromethane; DEA for diethylamine; DIPEA or DIEA for diisopropylethylamine; DMA for dimethylacetamide; DMF for N,N-dimethylformamide; DMSO for dimethylsulfoxide; dppf for 1,1′-bis(diphenylphosphino)ferrocene; Et for ethyl; EtOAc for ethyl acetate; EtOH for ethanol; h for hours; HDMS for hexamethyldisilazide; IPA for isopropyl alcohol; min for minutes; LiHDMS for lithium hexamethyldisilazide; MOM for methoxymethyl; Me for methyl; MeCN or ACN for acetonitrile; MeOH for methanol; NIS for N-iodosuccinimide; OAc for acetate; Ph for phenyl; PMB for paramethoxybenzyl; RT or rt for room temperature or retention time (context will dictate): Tos for tosyl; TBAF for tetrabutylammonium fluoride; TEA or Et3N for trimethylamine; TES-H for treithylsilane; TFA for trifluoroacetic acid; and THE for tetrahydrofuran.

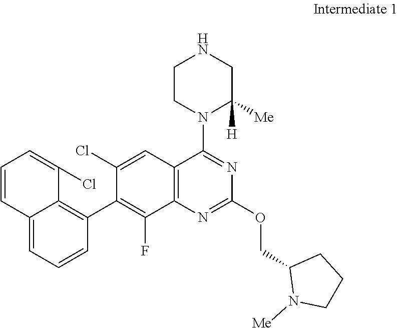 [SMILES: C[C@H]1CNCCN1c1nc(OC[C@@H]2CCCN2C)nc2c(F)c(-c3cccc4cccc(Cl)c34)c(Cl)cc12]

### 6-chloro-7-(8-chloronaphthalen-1-yl)-8-fluoro-4-((S)-2-methylpiperazin-1-yl)-2-(((S)-1-methylpyrrolidin-2-yl)methoxy)quinazoline

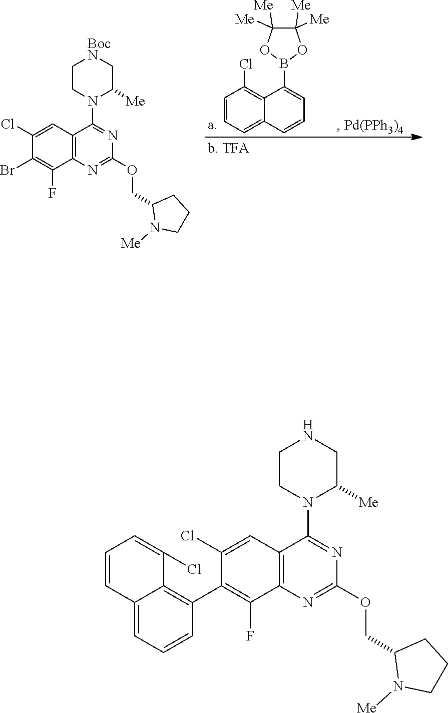

### Step 1: tert-butyl (3S)-4-(6-chloro-7-(8-chloronaphthalen-1-yl)-8-fluoro-2-(((S)-1-methylpyrrolidin-2-yl)methoxy)quinazolin-4-yl)-3-methylpiperazine-1-carboxylate

tert-Butyl (S)-4-(7-bromo-6-chloro-8-fluoro-2-(((S)-1-methylpyrrolidin-2-yl)methoxy)quinazolin-4-yl)-3-methylpiperazine-1-carboxylate (prepared according to the procedure found in U.S. Pat. No. 11,236,068, 46 mg, 0.08 mmol), 2-(8-chloronaphthalen-1-yl)-4,4,5,5-tetramethyl-1,3,2-dioxaborolane (35 mg, 0.12 mmol), sodium carbonate (43 mg, 0.4 mmol), and tetrakis triphenylphosphine palladium(0) (9 mg, 0.008 mmol) were combined as solids in a microwave vial. The vial was sealed. The atmosphere was evacuated and replaced with nitrogen. This process was performed three times. Degassed dioxane (0.9 mL) and water (0.3 mL) were added and the reaction mixture was heated at 100° C. overnight. The reaction mixture was concentrated and the crude residue was directly purified by column chromatography (0→100% EtOAc/hexanes) to provide the desired product (33 mg, 0.05 mmol, 63% yield). LC/MS (ESI) m/z: [M+H]⁺ calcd for C₃₄H₃₉Cl₂FN₅O₃ 654.2. found 654.2.

### Step 2: 6-chloro-7-(8-chloronaphthalen-1-yl)-8-fluoro-4-((S)-2-methylpiperazin-1-yl)-2-(((S)-1-methylpyrrolidin-2-yl)methoxy)quinazoline

tert-Butyl (3S)-4-(6-chloro-7-(8-chloronaphthalen-1-yl)-8-fluoro-2-(((S)-1-methylpyrrolidin-2-yl)methoxy)quinazolin-4-yl)-3-methylpiperazine-1-carboxylate (from Step 1, 33 mg, 0.050 mmol) was dissolved in DCM (0.5 mL) and TFA (0.2 mL). The reaction mixture was stirred at room temperature for 15 min at which point LC/MS analysis showed complete conversion to a product of desired mass. The reaction mixture was directly concentrated and used in the next step without additional purification (quantitative yield assumed). LC/MS (ESI) m/z: [M+H]⁺ calcd for C₂₉H₃₁Cl₂FN₅O₃ 554.2. found 554.1.

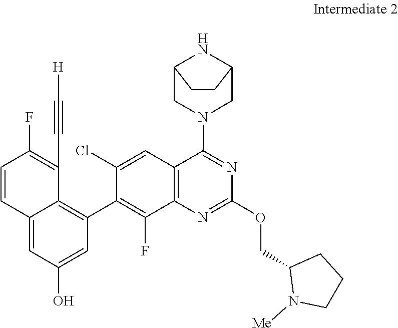 [SMILES: C#Cc1c(F)ccc2cc(O)cc(-c3c(Cl)cc4c(N5CC6CCC(C5)N6)nc(OC[C@@H]5CCCN5C)nc4c3F)c12]

### 4-(4-((1R,5S)-3,8-diazabicyclo[3.2.1]octan-3-yl)-6-chloro-8-fluoro-2-(((S)-1-methylpyrrolidin-2-yl)methoxy)quinazolin-7-yl)-5-ethynyl-6-fluoronaphthalen-2-ol

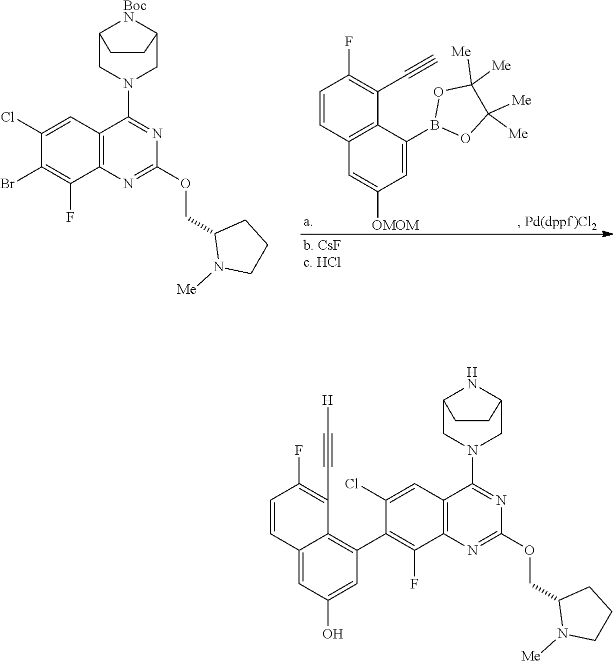

### Step 1: tert-butyl (1R,5S)-3-(6-chloro-8-fluoro-7-(7-fluoro-3-(methoxymethoxy)-8-((triisopropylsilyl)ethynyl)naphthalen-1-yl)-2-(((S)-1-methylpyrrolidin-2-yl)methoxy)quinazolin-4-yl)-3,8-diazabicyclo[3.2.1]octane-8-carboxylate

A suspension of tert-butyl (1R,5S)-3-(7-bromo-6-chloro-8-fluoro-2-(((S)-1-methylpyrrolidin-2-yl)methoxy)quinazolin-4-yl)-3,8-diazabicyclo[3.2.1]octane-8-carboxylate (prepared according to the procedures described in WO 2022/002102, 0.25 g, 0.43 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (prepared according to the procedures described in WO 2021/041671, 0.26 g, 0.51 mmol), and sodium carbonate (0.11 g, 1.1 mmol) in dioxane (12 mL) and water (4 mL) was degassed for 15 min by bubbling through nitrogen gas. 1,1′-bis(diphenylphosphino)ferrocene dichloropalladium(II) (0.03 g, 0.04 mmol) was added and the red suspension was heated at 100° C. for 24 h. The dark suspension was diluted with EtOAc (20 mL) and washed with water (10 mL) and brine (10 mL). The organic phase was dried over magnesium sulfate, filtered, and concentrated. The crude residue was purified by column chromatography (0→10% MeOH/DCM) to provide the desired product (110 mg, 0.12 mmol, 29% yield). LC/MS (ESI) m/z: [M+H]⁺ calcd for C₄₈H₆₃ClF₂N₅O₅Si 890.4. found 890.5.

### Step 2: tert-butyl (1R,5S)-3-(6-chloro-7-(8-ethynyl-7-fluoro-3-(methoxymethoxy)naphthalen-1-yl)-8-fluoro-2-(((S)-1-methylpyrrolidin-2-yl)methoxy)quinazolin-4-yl)-3,8-diazabicyclo[3.2.1]octane-8-carboxylate

tert-Butyl (1R,5S)-3-(6-chloro-8-fluoro-7-(7-fluoro-3-(methoxymethoxy)-8-((triisopropylsilyl)ethynyl)naphthalen-1-yl)-2-(((S)-1-methylpyrrolidin-2-yl)methoxy)quinazolin-4-yl)-3,8-diazabicyclo[3.2.1]octane-8-carboxylate (from Step 1, 135 mg, 0.152 mmol) was dissolved in DMF (2 mL) and solid CsF (69 mg, 0.46 mmol) was added. The reaction mixture was heated at 60° C. for 30 min. The reaction mixture was cooled to room temperature and was diluted with EtOAc (10 mL) and water (10 mL). The layers were separated and the organic phase was further washed with brine (10 mL), dried over magnesium sulfate, filtered, and concentrated. The crude residue was of sufficient purity to be used directly in the next step, without additional purification (quantitative yield assumed). LC/MS (ESI) m/z: [M+H]⁺ calcd for C₃₉H₄₃ClF₂N₅O₅ 734.3. found 734.3.

### Step 3: 4-(4-((1R,5S)-3,8-diazabicyclo[3.2.1]octan-3-yl)-6-chloro-8-fluoro-2-(((S)-1-methylpyrrolidin-2-yl)methoxy)quinazolin-7-yl)-5-ethynyl-6-fluoronaphthalen-2-ol

tert-Butyl (1R,5S)-3-(6-chloro-7-(8-ethynyl-7-fluoro-3-(methoxymethoxy)naphthalen-1-yl)-8-fluoro-2-(((S)-1-methylpyrrolidin-2-yl)methoxy)quinazolin-4-yl)-3,8-diazabicyclo[3.2.1]octane-8-carboxylate (from Step 2, 80 mg, 0.11 mmol) was dissolved in dioxane (0.5 mL) and HCl solution (4.0 M in dioxane, 1 mL, 4.0 mmol) was added. The reaction mixture was stirred at room temperature for 1 h. The mixture was directly concentrated and the crude residue was purified by column chromatography (0→15% MeOH/DCM) to provide the desired product as the HCl salt (60 mg, 0.095 mmol, 88% yield). LC/MS (ESI) m/z: [M+H]⁺ calcd for C₃₂H₃₁ClF₂N₅O₅ 590.2. found 590.4.

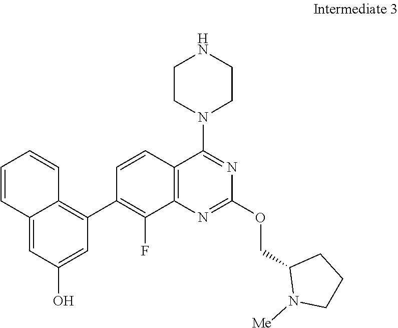 [SMILES: CN1CCC[C@H]1COc1nc(N2CCNCC2)c2ccc(-c3cc(O)cc4ccccc34)c(F)c2n1]

### (S)-4-(8-fluoro-2-((1-methylpyrrolidin-2-yl)methoxy)-4-(piperazin-1-yl)quinazolin-7-yl)naphthalen-2-ol

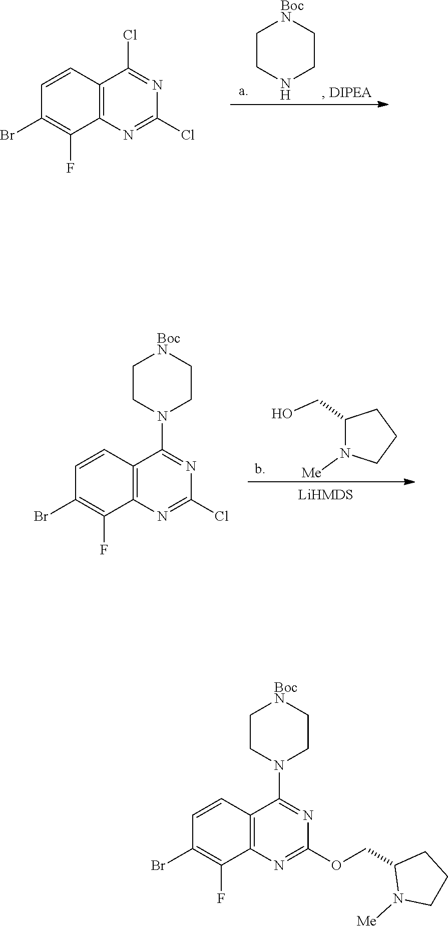

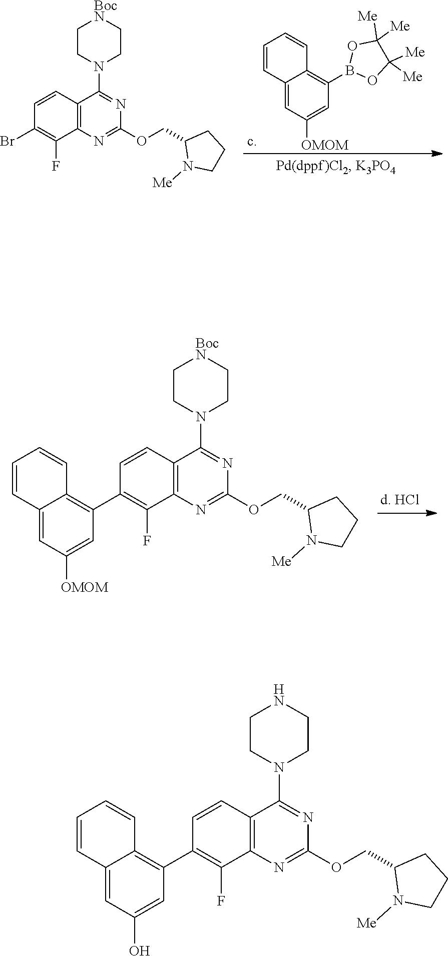

### Step 1: tert-butyl 4-(7-bromo-2-chloro-8-fluoroquinazolin-4-yl)piperazine-1-carboxylate

7-Bromo-2,4-dichloro-8-fluoroquinazoline (250 mg, 0.85 mmol) was dissolved in dioxane (15 ml) and DIPEA (0.44 mL, 2.5 mmol) was added followed by tert-butyl piperazine-1-carboxylate (160 mg, 0.85 mmol). The reaction mixture was stirred at room temperature for 2 h. The mixture was diluted with EtOAc (20 mL) and was quenched by addition of a NaOH solution (1.0 M, 2 mL). The layers were separated and the organic phase was washed with brine (10 mL). The combined aqueous layers were back extracted with EtOAc (2×20 mL). The combined organic layers were dried over magnesium sulfate, filtered, and concentrated. The crude residue was purified by column chromatography (5→45% EtOAc/hexanes) to provide the desired product (230 mg, 0.52 mmol, 61% yield) as a white solid. LC/MS (ESI) m/z: [M+H]⁺ calcd for C₁₇H₂₀BrClFN₄O₂ 445.0. found 444.8. NMR: ¹H NMR (500 MHz, CD₃OD) δ 7.80 (dd, J=9.1, 1.6 Hz, 1H), 7.68 (dd, J=9.1, 6.5 Hz, 1H), 3.99-3.93 (m, 4H), 3.69-3.63 (m, 4H), 1.49 (s, 9H).

### Step 2: tert-butyl (S)-4-(7-bromo-8-fluoro-2-((1-methylpyrrolidin-2-yl)methoxy)quinazolin-4-yl)piperazine-1-carboxylate

tert-Butyl 4-(7-bromo-2-chloro-8-fluoroquinazolin-4-yl)piperazine-1-carboxylate (from Step 1, 250 mg, 0.56 mmol) and (S)-(1-methylpyrrolidin-2-yl)methanol (194 mg, 1.68 mmol), were suspended in THE (4 mL). A solution of LiHMDS (1.0 M in THF, 0.62 mL, 0.62 mmol) was added dropwise and the reaction mixture was heated at 65° C. for 3 h. The mixture was diluted with EtOAc (20 mL) and was washed with water (10 mL) and brine (10 mL). The organic phase was dried over magnesium sulfate, filtered, and concentrated. The crude residue was purified by column chromatogrpahy (0→8% MeOH/DCM) to provide the desired product (120 mg, 0.23 mmol, 41% yield) as a white solid. LC/MS (ESI) m/z: [M+H]⁺ calcd for C₂₃H₃₂BrFN₅O₃ 524.2. found 524.1. NMR: ¹H NMR (500 MHz, CD₃OD) δ 7.70 (dd, J=9.1, 1.5 Hz, 1H), 7.47 (dd, J=9.1, 6.4 Hz, 1H), 4.50 (br dd, J=11.0, 5.8 Hz, 1H), 4.44 (dd, J=11.0, 5.6 Hz, 1H), 3.90-3.84 (m, 4H), 3.69-3.61 (m, 4H), 3.14-3.06 (m, 1H), 2.85-2.78 (m, 1H), 2.53 (s, 3H), 2.42-2.34 (m, 1H), 2.17-2.07 (m, 1H), 1.88-1.80 (m, 2H), 1.80-1.73 (m, 1H), 1.49 (s, 9H).

### Step 3: tert-butyl (S)-4-(8-fluoro-7-(3-(methoxymethoxy)naphthalen-1-yl)-2-((1-methylpyrrolidin-2-yl)methoxy)quinazolin-4-yl)piperazine-1-carboxylate

tert-Butyl (S)-4-(7-bromo-8-fluoro-2-((1-methylpyrrolidin-2-yl)methoxy)quinazolin-4-yl)piperazine-1-carboxylate (from Step 2, 120 mg, 0.23 mmol) 2-(3-(methoxymethoxy)naphthalen-1-yl)-4,4,5,5-tetramethyl-1,3,2-dioxaborolane (72 mg, 0.23 mmol), and potassium phosphate tribasic solution (2.0 M, 0.34 mL, 0.67 mmol) were dissolved in dioxane (2 mL). The solution was purged with a stream of nitrogen for 15 min. [1,1′-bis(di-tert-butylphosphino)ferrocene]dichloropalladium(II) (7.5 mg, 0.01 mmol) was added and the resulting solution was heated at 100° C. for 24 h. The reaction mixture was cooled to room temperature and diluted with EtOAc (20 mL). The organic phase was washed with water (10 mL) and brine (10 mL), dried over magnesium sulfate, filtered, and concentrated. The crude residue was purified by column chromatography (0→6% MeOH/DCM to provide the desired product (120 mg, 0.19 mmol, 83% yield) as an off-white solid. LC/MS (ESI) m/z: [M+H]⁺ calcd for C₃₅H₄₃FN₅O₅ 632.3; found 632.2. NMR: ¹H NMR (500 MHz, CD₃OD) δ 7.91 (d, J=8.6 Hz, 1H), 7.86 (d, J=8.3 Hz, 1H), 7.55 (d, J=2.4 Hz, 1H), 7.52-7.45 (m, 2H), 7.35-7.29 (m, 2H), 7.25 (d, J=2.5 Hz, 1H), 5.36 (s, 2H), 4.57-4.51 (m, 1H), 4.50-4.43 (m, 1H), 4.00-3.91 (m, 4H), 3.75-3.67 (m, 4H), 3.53 (s, 3H), 3.14-3.09 (m, 1H), 2.88-2.79 (m, 1H), 2.55 (s, 3H), 2.42-2.35 (m, 1H), 2.18-2.09 (m, 1H), 1.90-1.81 (m, 2H), 1.80-1.75 (m, 1H), 1.51 (s, 9H).

### Step 4: (S)-4-(8-fluoro-2-((1-methylpyrrolidin-2-yl)methoxy)-4-(piperazin-1-yl)quinazolin-7-yl)naphthalen-2-ol

tert-Butyl (S)-4-(8-fluoro-7-(3-(methoxymethoxy)naphthalen-1-yl)-2-((1-methylpyrrolidin-2-yl)methoxy)quinazolin-4-yl)piperazine-1-carboxylate (from Step 3, 120 mg, 0.19 mmol) was suspended in HCl solution (4.0 M in dioxane, 2 mL, 8.0 mmol) and was stirred at room temperature for 1 h. The reaction mixture was concentrated and azeotroped from DCM (3×5 mL). The crude residue was further dried under high vacuum to provide the desired product as its HCl salt (quantitative yield assumed). The crude material was of sufficient purity to be used directly in the next step, without additional purification. LC/MS (ESI) m/z: [M+H]⁺ calcd for C₂₈H₃₁FN₅O₂ 488.2. found 488.1.

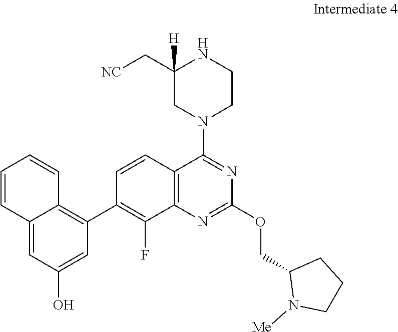 [SMILES: CN1CCC[C@H]1COc1nc(N2CCN[C@@H](CC#N)C2)c2ccc(-c3cc(O)cc4ccccc34)c(F)c2n1]

### 2-((S)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile

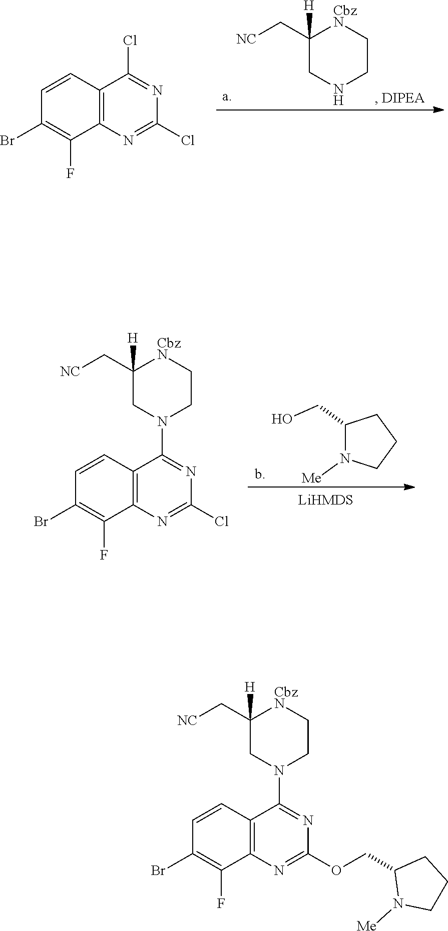

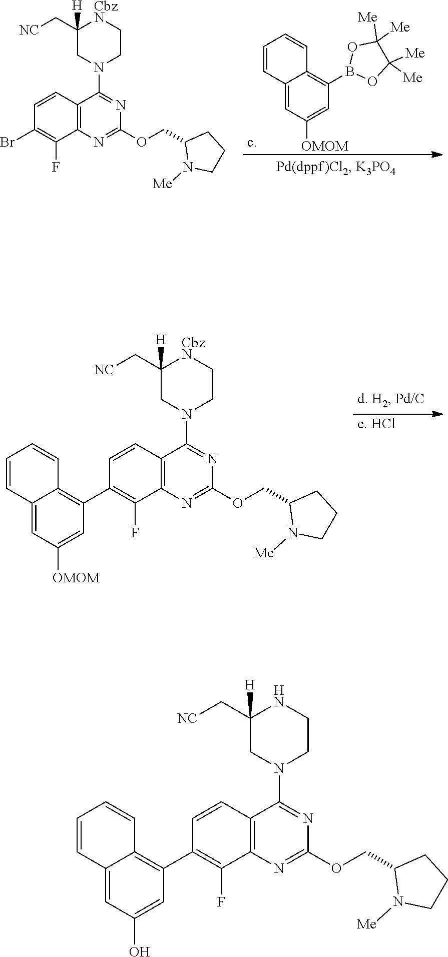

### Step 1: Benzyl (S)-4-(7-bromo-2-chloro-8-fluoroquinazolin-4-yl)-2-(cyanomethyl)piperazine-1-carboxylate

7-Bromo-2,4-dichloro-8-fluoroquinazoline (500 mg, 1.7 mmol) was dissolved in dioxane (15 ml) and DIPEA (0.89 mL, 5.1 mmol) was added followed by benzyl (S)-2-(cyanomethyl)piperazine-1-carboxylate (440 mg, 1.7 mmol). The reaction mixture was stirred at room temperature for 2 h. The mixture was diluted with EtOAc (20 mL) and was quenched by addition of a NaOH solution (1.0 M, 2 mL). The layers were separated and the organic phase was washed with brine (10 mL). The combined aqueous layers were back extracted with EtOAc (2×20 mL). The combined organic layers were dried over magnesium sulfate, filtered, and concentrated. The crude residue was purified by column chromatography (5→50% EtOAc/hexanes) to provide the desired product (760 mg, 1.5 mmol, 87% yield) as a white solid. LC/MS (ESI) m/z: [M+H]⁺ calcd for C₂₂H₁₉BrClFN₅O₂518.0. found 517.9. NMR: ¹H NMR (500 MHz, CD₃OD) δ 7.80 (dd, J=9.1, 1.5 Hz, 1H), 7.68 (dd, J=9.1, 6.5 Hz, 1H), 7.45-7.28 (m, 5H), 5.24-5.12 (m, 2H), 4.79-4.71 (m, 1H), 4.50-4.34 (m, 2H), 4.14-4.08 (m, 1H), 3.81-3.70 (m, 1H), 3.69-3.61 (m, 1H), 3.59-3.46 (m, 1H), 3.04-2.88 (m, 2H).

### Step 2: benzyl (S)-4-(7-bromo-8-fluoro-2-(((S)-1-methylpyrrolidin-2-yl)methoxy)quinazolin-4-yl)-2-(cyanomethyl)piperazine-1-carboxylate

Benzyl (S)-4-(7-bromo-2-chloro-8-fluoroquinazolin-4-yl)-2-(cyanomethyl)piperazine-1-carboxylate (from Step 1, 150 mg, 0.29 mmol) and (S)-(1-methylpyrrolidin-2-yl)methanol (100 mg, 0.87 mmol) were suspended in THE (8 mL). A solution of LiHMDS (1.0 M in THF, 0.32 mL, 0.32 mmol) was added dropwise and the reaction mixture was heated at 65° C. for 5 h. The mixture was diluted with EtOAc (20 mL) and was washed with water (10 mL) and brine (10 mL). The organic phase was dried over magnesium sulfate, filtered, and concentrated. The crude residue was purified by column chromatogrpahy (0→8% MeOH/DCM) to provide the desired product (139 mg, 0.23 mmol, 80% yield) as a white solid. LC/MS (ESI) m/z: [M+H]⁺ calcd for C₂₈H₃₁BrFN₆O₃ 597.2. found 59.0. 1H NMR (500 MHz, CD₃OD) δ 7.72 (dd, J=9.1, 1.4 Hz, 1H), 7.48 (dd, J=9.0, 6.4 Hz, 1H), 7.42-7.29 (m, 5H), 5.25-5.14 (m, 2H), 4.79-4.72 (m, 1H), 4.50 (br dd, J=11.0, 6.0 Hz, 1H), 4.45 (dd, J=11.0, 5.2 Hz, 1H), 4.39-4.28 (m, 2H), 4.15-4.08 (m, 1H), 3.73-3.67 (m, 1H), 3.54-3.49 (m, 1H), 3.14-3.09 (m, 1H), 3.04-2.90 (m, 2H), 2.86-2.79 (m, 1H), 2.54 (s, 3H), 2.41-2.36 (m, 1H), 2.16-2.07 (m, 1H), 1.89-1.81 (m, 2H), 1.80-1.73 (m, 1H).

### Step 3: benzyl (S)-2-(cyanomethyl)-4-(8-fluoro-7-(3-(methoxymethoxy)naphthalen-1-yl)-2-(((S)-1-methylpyrrolidin-2-yl)methoxy)quinazolin-4-yl)piperazine-1-carboxylate

Benzyl (S)-4-(7-bromo-8-fluoro-2-(((S)-1-methylpyrrolidin-2-yl)methoxy)quinazolin-4-yl)-2-(cyanomethyl)piperazine-1-carboxylate (from Step 2, 320 mg, 0.536 mmol), 2-(3-(methoxymethoxy)naphthalen-1-yl)-4,4,5,5-tetramethyl-1,3,2-dioxaborolane (168 mg, 0.54 mmol), and potassium phosphate tribasic solution (2.0 M, 0.80 mL, 1.6 mmol) were dissolved in dioxane (5 mL). The solution was purged with a stream of nitrogen for 15 min. [1,1′-bis(di-tert-butylphosphino)ferrocene]dichloropalladium(II) (17 mg, 0.03 mmol) was added and the resulting solution was heated at 100° C. for 3 h. The reaction mixture was cooled to room temperature and diluted with EtOAc (20 mL). The organic phase was washed with water (10 mL) and brine (10 mL), dried over magnesium sulfate, filtered, and concentrated. The crude residue was purified by column chromatography (0→6% MeOH/DCM to provide the desired product (243 mg, 0.34 mmol, 64% yield) as an off-white solid. LC/MS (ESI) m/z: [M+H]⁺ calcd for C₄₀H₄₂FN₆O₅ 705.3. found 705.3. NMR: ¹H NMR (500 MHz, CD₃OD) δ 7.93 (d, J=8.8 Hz, 1H), 7.86 (d, J=8.1 Hz, 1H), 7.55 (d, J=2.3 Hz, 1H), 7.53-7.45 (m, 2H), 7.43-7.29 (m, 7H), 7.25 (d, J=2.5 Hz, 1H), 5.36 (s, 2H), 5.27-5.16 (m, 2H), 4.56-4.50 (m, 1H), 4.49-4.37 (m, 3H), 4.20-4.14 (m, 1H), 3.80 (s, 1H), 3.61-3.55 (m, 1H), 3.53 (s, 3H), 3.12-2.95 (m, 3H), 2.85-2.78 (m, 1H), 2.53 (s, 3H), 2.40-2.32 (m, 1H), 2.15-2.08 (m, 1H), 1.88-1.80 (m, 2H), 1.79-1.74 (m, 1H).

### Step 4: 2-((S)-4-(8-fluoro-7-(3-(methoxymethoxy)naphthalen-1-yl)-2-(((S)-1-methylpyrrolidin-2-yl)methoxy)quinazolin-4-yl)piperazin-2-yl)acetonitrile

Benzyl (S)-2-(cyanomethyl)-4-(8-fluoro-7-(3-(methoxymethoxy)naphthalen-1-yl)-2-(((S)-1-methylpyrrolidin-2-yl)methoxy)quinazolin-4-yl)piperazine-1-carboxylate (from Step 3, 240 mg, 0.34 mmol) and palladium on carbon (10 wt. %, 36 mg, 0.03 mmol) were suspended in ethanol (20 mL). Hydrogen gas was bubbled through the reaction mixture for 5 min. The reaction mixture was kept under a positive pressure of hydrogen (1 atm, balloon) for 2.5 h. The reaction mixture was filtered through diatomaceous earth (Celite™), taking care not to allow the filter cake to go dry, and the filtrate was concentrated. The crude residue was of sufficient purity to be used directly in the next step, without additional purification (quantitative yield assumed). LC/MS (ESI) m/z: [M+H]⁺ calcd for C₃₂H₃₆FN₆O₃ 571.3. found 571.2. ¹H NMR (500 MHz, CD₃OD) δ 7.88 (d, J=8.7 Hz, 1H), 7.85 (d, J=8.2 Hz, 1H), 7.54 (d, J=2.4 Hz, 1H), 7.52-7.45 (m, 2H), 7.35-7.28 (m, 2H), 7.24 (d, J=2.4 Hz, 1H), 5.36 (s, 2H), 4.57-4.44 (m, 3H), 4.40-4.33 (m, 1H), 3.53 (s, 3H), 3.51-3.41 (m, 1H), 3.23-2.99 (m, 4H), 2.88-2.79 (m, 1H), 2.73-2.68 (m, 2H), 2.54 (s, 3H), 2.41-2.35 (m, 1H), 2.19-2.08 (m, 1H), 1.88-1.74 (m, 3H).

### Step 5: 2-((S)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile

2-((S)-4-(8-Fluoro-7-(3-(methoxymethoxy)naphthalen-1-yl)-2-(((S)-1-methylpyrrolidin-2-yl)methoxy)quinazolin-4-yl)piperazin-2-yl)acetonitrile (from Step 4, 298 mg, 0.52 mmol) was suspended in HCl solution (4.0 M in dioxane, 2 mL, 8.0 mmol) and the reaction mixture was stirred at room temperature for 1 h. The mixture was directly concentrated and further dried on high vacuum to provide the desired product as the HCl salt (quantitative yield assumed). The crude material was of sufficient purity to be used directly in the next step, without additional purification. LC/MS (ESI) m/z: [M+H]⁺ calcd for C₃₀H₃₁FN₆O₂ 527.3. found 527.2. ¹H NMR (500 MHz, CD₃OD) δ 8.17 (br d, J=8.8 Hz, 1H), 7.78 (d, J=8.2 Hz, 1H), 7.66 (br d, J=5.6 Hz, 1H), 7.49-7.42 (m, 2H), 7.30 (d, J=2.3 Hz, 1H), 7.27-7.22 (m, 1H), 7.15 (br d, J=3.8 Hz, 1H), 5.22-5.09 (m, 2H), 5.03-4.88 (m, 3H), 4.39-4.22 (m, 2H), 4.14-3.95 (m, 2H), 3.83-3.77 (m, 1H), 3.76-3.72 (m, 1H), 3.67-3.65 (m, 5H), 3.61-3.55 (m, 2H), 2.52-2.40 (m, 1H), 2.29-2.15 (m, 2H), 2.14-2.07 (m, 1H).

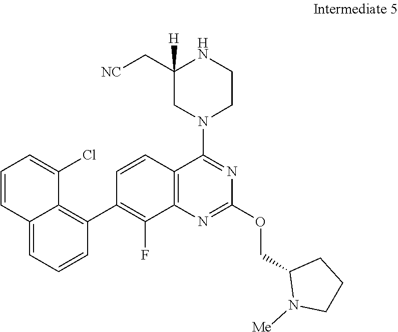 [SMILES: CN1CCC[C@H]1COc1nc(N2CCN[C@@H](CC#N)C2)c2ccc(-c3cccc4cccc(Cl)c34)c(F)c2n1]

### 2-((S)-4-(8-fluoro-7-(8-chloronaphthalen-1-yl)-2-(((S)-1-methylpyrrolidin-2-yl)methoxy)quinazolin-4-yl)piperazin-2-yl)acetonitrile

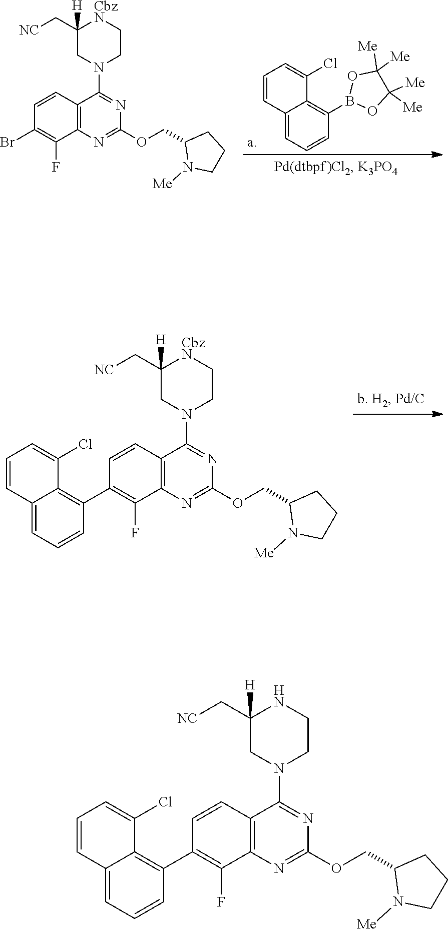

### Step 1: benzyl (S)-2-(cyanomethyl)-4-(8-fluoro-7-(8-chloronaphthalen-1-yl)-2-(((S)-1-methylpyrrolidin-2-yl)methoxy)quinazolin-4-yl)piperazine-1-carboxylate

Benzyl (S)-4-(7-bromo-8-fluoro-2-(((S)-1-methylpyrrolidin-2-yl)methoxy)quinazolin-4-yl)-2-(cyanomethyl)piperazine-1-carboxylate (from Step 2 of Intermediate 4, 85 mg, 0.20 mmol, 2-(8-chloronaphthalen-1-yl)-4,4,5,5-tetramethyl-1,3,2-dioxaborolane (120 mg, 0.20 mmol), and potassium phosphate tribasic solution (2.0 M, 0.30 mL, 0.60 mmol) were dissolved in dioxane (4 mL). The solution was purged with a stream of nitrogen for 15 min. [1,1′-bis(di-tert-butylphosphino)ferrocene]dichloropalladium(II) (6.5 mg, 0.01 mmol) was added and the resulting solution was heated at 100° C. for 2.5 h. The reaction mixture was cooled to room temperature and diluted with EtOAc (20 mL). The organic phase was washed with water (10 mL) and brine (10 mL), dried over magnesium sulfate, filtered, and concentrated. The crude residue was purified by column chromatography (0→6% MeOH/DCM to provide the desired product (50 mg, 0.07 mmol, 37% yield) as an off-white solid. LC/MS (ESI) m/z: [M+H]⁺ calcd for C₃₈H₃₇ClFN₆O₅ 679.3. found 679.3. ¹H NMR (500 MHz, CD₃OD) δ 8.07 (dd, J=8.2, 1.1 Hz, 1H), 7.98 (d, J=8.1 Hz, 1H), 7.86 (d, J=8.7 Hz, 1H), 7.63 (dd, J=8.1, 7.3 Hz, 1H), 7.57 (dd, J=7.4, 1.2 Hz, 1H), 7.50-7.46 (m, 2H), 7.43-7.30 (m, 6H), 5.27-5.15 (m, 2H), 4.57-4.29 (m, 4H), 4.20-4.14 (m, 1H), 3.77-3.67 (m, 1H), 3.61-3.49 (m, 1H), 3.15-2.94 (m, 3H), 2.87-2.79 (m, 1H), 2.54 (s, 3H), 2.43-2.37 (m, 1H), 2.17-2.08 (m, 1H), 1.88-1.82 (m, 2H), 1.80-1.76 (m, 1H).

### Step 2: 2-((S)-4-(8-fluoro-7-(8-chloronaphthalen-1-yl)-2-(((S)-1-methylpyrrolidin-2-yl)methoxy)quinazolin-4-yl)piperazin-2-yl)acetonitrile

Benzyl (S)-2-(cyanomethyl)-4-(8-fluoro-7-(8-chloronaphthalen-1-yl)-2-(((S)-1-methylpyrrolidin-2-yl)methoxy)quinazolin-4-yl)piperazine-1-carboxylate (from Step 1, 50 mg, 0.07 mmol) and palladium on carbon (10 wt. %, 78 mg, 0.07 mmol) were suspended in ethanol (10 mL). Hydrogen gas was bubbled through the reaction mixture for 5 min. The reaction mixture was kept under a positive pressure of hydrogen (1 atm, balloon) for 1.5 h. The reaction mixture was filtered through diatomaceous earth (Celite™), taking care not to allow the filter cake to go dry, and the filtrate was concentrated. The crude residue was of sufficient purity to be used directly in the next step, without additional purification (quantitative yield assumed). LC/MS (ESI) m/z: [M+H]⁺ calcd for C₃₀H₃₁ClFN₆O 545.2. found 545.4.

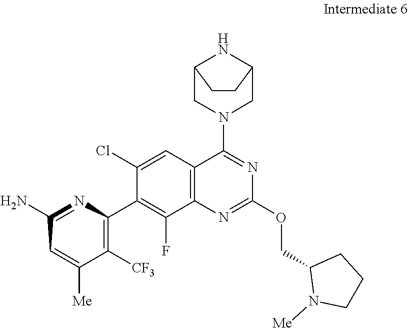

### 6-(6-chloro-4-{3,8-diazabicyclo[3.2.1]octan-3-yl}-8-fluoro-2-{[(2S)-1-methylpyrrolidin-2-yl]methoxy}quinazolin-7-yl)-4-methyl-5-(trifluoromethyl)pyridin-2-amine

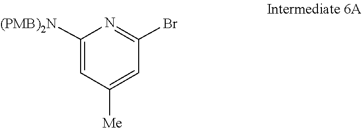

### 6-bromo-N,N-bis(4-methoxybenzyl)-4-methylpyridin-2-amine

To a solution of 6-bromo-4-methylpyridin-2-amine (1 g, 5.35 mmol) in DMF (20 mL) was added NaH (0.64 g, 16 mmol, 60%) at 0° C. The mixture was stirred at 0° C. for 0.5 h. Then 1-(chloromethyl)-4-methoxybenzene (2.1 g, 13.4 mmol) was added. The mixture was stirred at 0° C. for 1.5 h. TLC (on silica gel, petroleum ether:ethyl acetate=5:1) showed the reaction was completed. The reaction was quenched with saturated NH₄C₁ (20 mL) and extracted with ethyl acetate (20 mL×3), washed with brine (50 mL) and dried over anhydrous Na₂SO₄. The mixture was filtered. The filtrate was concentrated in vacuo. The residue was purified by column chromatography (silica gel, petroleum ether:ethyl acetate=5:1) to provide 6-bromo-N,N-bis(4-methoxybenzyl)-4-methylpyridin-2-amine (2 g, 4.68 mmol, 87.5% yield) as a colorless oil. ¹H NMR (400 MHz, CDCl₃) δ 7.16 (d, J=8.8 Hz, 4H), 6.88-6.84 (m, 4H), 6.60 (s, 1H), 6.16 (s, 1H), 4.64 (s, 4H), 3.80 (s, 6H), 2.13 (s, 3H).

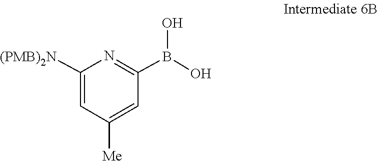

### (6-(bis(4-methoxybenzyl)amino)-4-methylpyridin-2-yl)boronic acid

To a solution of 6-bromo-N,N-bis(4-methoxybenzyl)-4-methylpyridin-2-amine (Intermediate 6A, 1000 mg, 2.34 mmol), bis(pinacolato)diboron (Intermediate 6A, 832.5 mg, 3.28 mmol), (1,1′-bis(diphenylphosphino)ferrocene)palladium(II) dichloride (171 mg, 0.23 mmol) in 1,4-dioxane (20 mL) was added KOAc (459.32 mg, 4.68 mmol). The mixture was stirred at 90° C. for 5 h under N₂. The reaction mixture was filtered. The filtrate containing the crude product (6-(bis(4-methoxybenzyl)amino)-4-methylpyridin-2-yl)boronic acid (918 mg, 2.34 mmol, crude) in 1,4-dioxane (20 mL)) was used in the next step without purification. MS (ESI) m/z 393.3 [M+1]⁺.

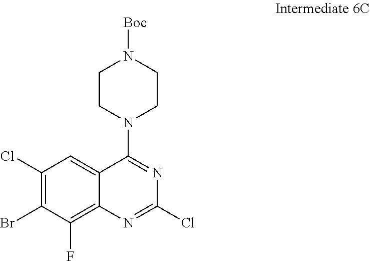 [SMILES: CC(C)(C)OC(=O)N1CCN(c2nc(Cl)nc3c(F)c(Br)c(Cl)cc23)CC1]

### tert-butyl 4-(7-bromo-2,6-dichloro-8-fluoroquinazolin-4-yl)piperazine-1-carboxylate

To a solution of 7-bromo-2,4,6-trichloro-8-fluoroquinazoline (1 g, 3.03 mmol) and DIPEA (1.32 mL, 7.57 mmol) in THE (15 mL) was added tert-butyl piperazine-1-carboxylate (0.56 g, 3.03 mmol) under N₂. The reaction mixture was stirred at 25° C. for 2 h. The mixture was concentrated. The residue was diluted with ethyl acetate (60 mL), washed with water (30 mL×2) and brine (50 mL). The organic phase was dried over anhydrous sodium sulphate, filtered and concentrated under reduced pressure. The residue was purified by silica gel column chromatography (silica gel, petroleum ether:ethyl acetate=10:1 to 4:1) to provide tert-butyl 4-(7-bromo-2,6-dichloro-8-fluoroquinazolin-4-yl)piperazine-1-carboxylate (1.33 g, 2.77 mmol, 91.5% yield) as a yellow solid. MS (ESI) m/z 481.0 [M+3]+. ¹H NMR (400 MHz, CDCl₃) δ 7.77 (d, J=1.6 Hz, 1H), 3.93-3.84 (m, 4H), 3.72-3.61 (m, 4H), 1.50 (s, 9H).

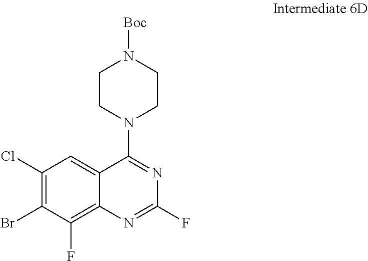 [SMILES: CC(C)(C)OC(=O)N1CCN(c2nc(F)nc3c(F)c(Br)c(Cl)cc23)CC1]

### tert-butyl 4-(7-bromo-6-chloro-2,8-difluoroquinazolin-4-yl)piperazine-1-carboxylate

A solution of tert-butyl 4-(7-bromo-2,6-dichloro-8-fluoroquinazolin-4-yl)piperazine-1-carboxylate (Intermediate 6C, 1000 mg, 2.08 mmol) and potassium fluoride (2420 mg, 41.65 mmol) in DMA (10 mL) was stirred at 110° C. for 12 h under N₂. The reaction mixture was quenched with water (30 mL) and extracted with EtOAc (30 mL×3). The combined organic layers were washed with brine (30 mL×3) and dried over anhydrous Na₂SO₄. The mixture was filtered, and the filtrate was concentrated under reduced pressure. The residue was purified by silica gel column chromatography (silica gel, petroleum ether:ethyl acetate=20:1 to 3:1) to provide tert-butyl 4-(7-bromo-6-chloro-2,8-difluoroquinazolin-4-yl)piperazine-1-carboxylate (730 mg, 1.57 mmol, 75.6% yield) as a yellow solid. MS (ESI) m/z 463.1 [M+1]⁺.

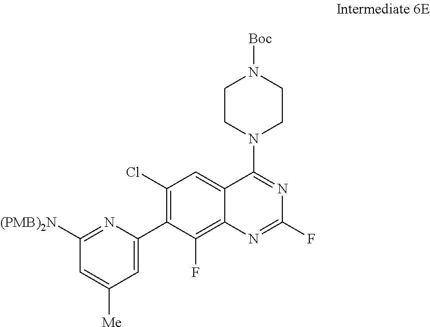

### tert-butyl 4-(7-(6-(bis(4-methoxybenzyl)amino)-4-methylpyridin-2-yl)-6-chloro-2,8-difluoroquinazolin-4-yl)piperazine-1-carboxylate

A solution of tert-butyl 4-(7-bromo-6-chloro-2,8-difluoroquinazolin-4-yl)piperazine-1-carboxylate (Intermediate 6D, 600 mg, 1.29 mmol), (6-(bis(4-methoxybenzyl)amino)-4-methylpyridin-2-yl)boronic acid (Intermediate 6B, 756 mg, 1.93 mmol), (1,1′-bis(diphenylphosphino)ferrocene)palladium(II) dichloride (104 mg, 0.14 mmol) and potassium phosphate (548 mg, 2.59 mmol) in 1,4-dioxane (20 mL) and water (2 mL) was stirred at 60° C. for 12 under N₂. The mixture was filtered. The filtrate was concentrated in vacuo. The residue was purified by silica gel column chromatography (silica gel, petroleum ether:ethyl acetate=10:1 to 3:1) to provide tert-butyl 4-(7-(4-(bis(4-methoxybenzyl)amino)-6-methylpyridin-2-yl)-6-chloro-2,8-difluoroquinazolin-4-yl)piperazine-1-carboxylate (600 mg, 0.82 mmol, 63.4% yield) as a yellow oil. MS (ESI) m/z 731.4 [M+1]⁺; ¹H NMR (400 MHz, CDCl₃) δ 7.76 (d, J=1.2 Hz, 1H), 7.18 (d, J=8.8 Hz, 4H), 6.85 (d, J=8.8 Hz, 4H), 6.59 (s, 1H), 6.37 (s, 1H), 4.69 (s, 4H), 3.98-3.87 (m, 4H), 3.80 (s, 6H), 3.69-3.65 (m, 4H), 2.27 (s, 3H), 1.51 (s, 9H).

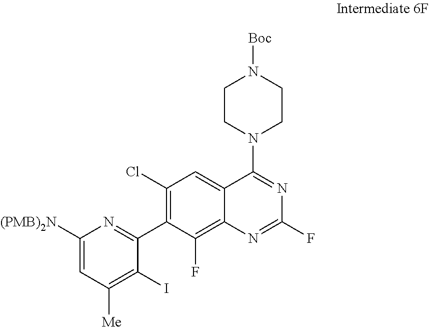

### tert-butyl 4-(7-(6-(bis(4-methoxybenzyl)amino)-3-iodo-4-methylpyridin-2-yl)-6-chloro-2,8-difluoroquinazolin-4-yl)piperazine-1-carboxylate

A solution of tert-butyl 4-(7-(4-(bis(4-methoxybenzyl)amino)-6-methylpyridin-2-yl)-6-chloro-2,8-difluoroquinazolin-4-yl)piperazine-1-carboxylate (Intermediate 6E, 800 mg, 1.09 mmol), TosOH (5 mg, 0.05 mmol) and NIS (1200 mg, 5.33 mmol) in DMF (10 mL) was stirred at 25° C. for 12 h. The reaction mixture was diluted with water (15 mL) and EtOAc (15 mL). The mixture was extracted with EtOAc (30 mL×3). The combined organic layers were washed with brine (30 mL×3), dried over anhydrous sodium sulfate, filtered and concentrated under reduced pressure. The residue was purified by column chromatography (silica gel, petroleum ether:ethyl acetate=10:1 to 3:1) to provide tert-butyl-4-(7-(6-(bis(4-methoxybenzyl)amino)-3-iodo-4-methylpyridin-2-yl)-6-chloro-2,8-difluoroquinazolin-4-yl)piperazine-1-carboxylate (400 mg, 0.467 mmol, 42.7% yield) as a yellow solid. MS (ESI) m/z 857.2 [M+1]⁺; ¹H NMR (400 MHz, CDCl₃) δ 7.82 (d, J=1.2 Hz, 1H), 7.17 (d, J=8.4 Hz, 4H), 6.86 (d, J=8.4 Hz, 4H), 6.48 (s, 1H), 4.76-4.65 (m, 2H), 4.62-4.50 (m, 2H), 4.01-3.92 (m, 4H), 3.82 (s, 6H), 3.72-3.63 (m, 4H), 2.38 (s, 3H), 1.52 (s, 9H).

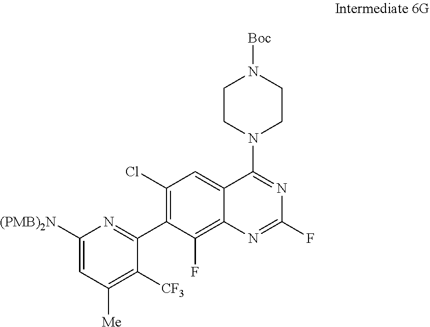

### tert-butyl 4-(7-(6-(bis(4-methoxybenzyl)amino)-4-methyl-3-(trifluoromethyl)pyridin-2-yl)-6-chloro-2,8-difluoroquinazolin-4-yl)piperazine-1-carboxylate

A mixture of tert-butyl 4-(7-(6-(bis(4-methoxybenzyl)amino)-3-iodo-4-methylpyridin-2-yl)-6-chloro-2,8-difluoroquinazolin-4-yl)piperazine-1-carboxylate (Intermediate 6F, 400 mg, 0.4700 mmol), methyl 2,2-difluoro-2-fluorosulfonyl-acetate (1345 mg, 7 mmol) and CuI (267 mg, 1.4 mmol) in DMA (10 mL) was stirred at 80° C. for 5 h under N₂. The reaction mixture was then cooled to room temperature and additional CuI (267 mg, 1.4 mmol) and methyl 2,2-difluoro-2-fluorosulfonyl-acetate (1345 mg, 7 mmol) were added to the mixture. The reaction mixture was stirred at 80° C. for another 12 h under N₂. The mixture was diluted with EtOAc (50 mL) and filtered. The filtrate was washed with brine (30 mL×3) and dried over anhydrous Na₂SO₄ and filtered. The filtrate was concentrated in vacuo. The residue was purified by column chromatography (silica gel, petroleum ether:ethyl acetate=10:1 to 3:1) to provide tert-butyl 4-(7-(6-(bis(4-methoxybenzyl)amino)-4-methyl-3-(trifluoromethyl)pyridin-2-yl)-6-chloro-2,8-difluoroquinazolin-4-yl)piperazine-1-carboxylate (270 mg, 0.34 mmol, 72.4% yield) as a yellow solid. MS (ESI) m/z 799.0 [M+1]⁺.

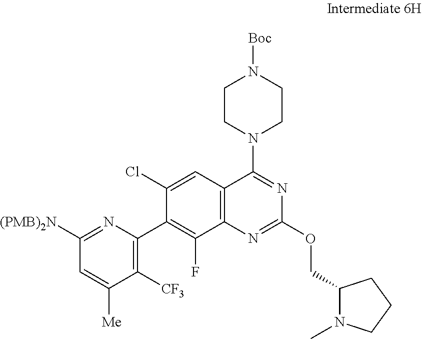

### tert-butyl 4-(7-(6-(bis(4-methoxybenzyl)amino)-4-methyl-3-(trifluoromethyl)pyridin-2-yl)-6-chloro-2,8-difluoroquinazolin-4-yl)piperazine-1-carboxylate

To a solution of (2S)-1-methylpyrrolidin-2-yl methanol (97.6 mg, 0.85 mmol) in THE (10 mL) was added NaH (81 mg, 2.03 mmol, 60%) at 0° C. The mixture was stirred at 0° C. for 0.5 h. tert-butyl 4-(7-(6-(bis(4-methoxybenzyl)amino)-4-methyl-3-(trifluoromethyl)pyridin-2-yl)-6-chloro-2,8-difluoroquinazolin-4-yl)piperazine-1-carboxylate (Intermediate 6G, 270 mg, 0.34 mmol) in THE (5 mL) was added. The mixture was stirred at 0° C. for 1 h. The reaction mixture was quenched with saturated NH₄C₁ (20 mL) and extracted with EtOAc (20 mL×3). The combined organic layers were washed with brine (30 mL) and dried over anhydrous Na₂SO₄. The mixture was filtered, the filtrate was concentrated under reduced pressure. The residue was purified by column chromatography (silica gel, DCM:MeOH=10:1) to provide tert-butyl 4-(7-(6-(bis(4-methoxybenzyl)amino)-4-methyl-3-(trifluoromethyl)pyridin-2-yl)-6-chloro-2,8-difluoroquinazolin-4-yl)piperazine-1-carboxylate (250 mg, 0.28 mmol, 82.7% yield) as a white solid. MS (ESI) m/z 894.5 [M+1]⁺.

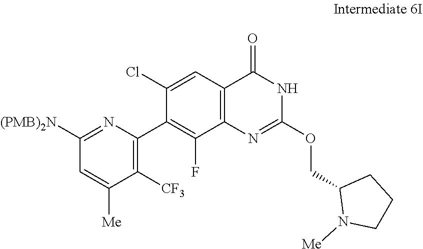

### 7-(6-(bis(4-methoxybenzyl)amino)-4-methyl-3-(trifluoromethyl)pyridin-2-yl)-6-chloro-8-fluoro-2-(((S)-1-methylpyrrolidin-2-yl)methoxy)quinazolin-4(3H)-one

A mixture of tert-butyl 4-(7-(6-(bis(4-methoxybenzyl)amino)-4-methyl-3-(trifluoromethyl)pyridin-2-yl)-6-chloro-2,8-difluoroquinazolin-4-yl)piperazine-1-carboxylate (Intermediate 6H, 250 mg, 0.28 mmol) and NaOH (224 mg, 5.59 mmol) in ethanol (30 mL) and water (10 mL) was stirred at 45° C. for 3 d. The mixture was quenched with 2N HCl to pH=6-7. The mixture was concentrated in vacuo to remove EtOH. The residue was extracted with DCM (20 mL×3). The combined organic layers were washed with brine (30 mL) and dried over anhydrous Na₂SO₄. The mixture was filtered and the filtrate was concentrated under reduced pressure to provide 7-(6-(bis(4-methoxybenzyl)amino)-4-methyl-3-(trifluoromethyl)pyridin-2-yl)-6-chloro-8-fluoro-2-(((S)-1-methylpyrrolidin-2-yl)methoxy)quinazolin-4(3H)-one (200 mg, 0.28 mmol, 98.5% yield) as a light yellow solid. MS (ESI) m/z 726.3 [M+1]⁺; ¹H NMR (400 MHz, CDCl₃) δ 8.07 (s, 1H), 7.14 (d, J=8.4 Hz, 4H), 6.85 (d, J=8.4 Hz, 4H), 6.41 (s, 1H), 4.98-4.65 (m, 4H), 4.59-4.49 (m, 2H), 3.80 (s, 6H), 3.55-3.38 (m, 1H), 2.90 (d, J=8.0 Hz, 3H), 2.41 (s, 3H), 2.31-2.21 (m, 2H), 2.14-2.03 (m, 2H), 1.37-1.19 (m, 2H).

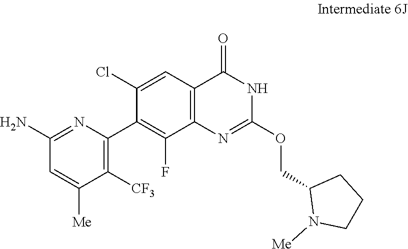 [SMILES: Cc1cc(N)nc(-c2c(Cl)cc3c(=O)[nH]c(OC[C@@H]4CCCN4C)nc3c2F)c1C(F)(F)F]

### 7-(6-amino-4-methyl-3-(trifluoromethyl)pyridin-2-yl)-6-chloro-8-fluoro-2-(((S)-1-methylpyrrolidin-2-yl)methoxy)quinazolin-4(3H)-one

A solution of 7-(6-(bis(4-methoxybenzyl)amino)-4-methyl-3-(trifluoromethyl)pyridin-2-yl)-6-chloro-8-fluoro-2-(((S)-1-methylpyrrolidin-2-yl)methoxy)quinazolin-4(3H)-one (Intermediate 61, 300. mg, 0.4100 mmol) in TFA (10 mL, 130 mmol) was stirred at 50° C. for 4 hours. The reaction mixture was concentrated under reduced pressure to provide the crude product 7-(6-amino-4-methyl-3-(trifluoromethyl)pyridin-2-yl)-6-chloro-8-fluoro-2-(((S)-1-methylpyrrolidin-2-yl)methoxy)quinazolin-4(3H)-one (247 mg, 0.41 mmol, 99.7% yield) as a brown oil. MS (ESI) m/z: 486.1 [M+H]⁺.

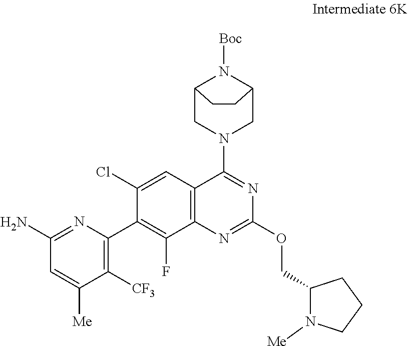 [SMILES: Cc1cc(N)nc(-c2c(Cl)cc3c(N4CC5CCC(C4)N5C(=O)OC(C)(C)C)nc(OC[C@@H]4CCCN4C)nc3c2F)c1C(F)(F)F]

### tert-butyl 3-(7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3,8-diazabicyclo[3.2.1]octane-8-carboxylate

To a solution of 7-(6-amino-4-methyl-3-(trifluoromethyl)pyridin-2-yl)-6-chloro-8-fluoro-2-(((S)-1-methylpyrrolidin-2-yl)methoxy)quinazolin-4(3H)-one (Intermediate 6J, 80 mg, 0.13 mmol) in DCM (3 mL) were added tert-butyl 3,8-diazabicyclo[3.2.1]octane-8-carboxylate (85 mg, 0.40 mmol), DIPEA (51 mg, 0.39 mmol) and BOP (192 mg, 0.43 mmol) at 25° C. The reaction mixture was stirred at 25° C. for 12 hours. The reaction mixture was diluted with water (15 mL), then extracted with DCM (15 mL×3). The combined organic layers were washed with brine (20 mL), dried over anhydrous sodium sulfate, filtered and concentrated under reduced pressure. The residue was purified by preparative HPLC (Instrument: ACSWH-GX-N; Column: Phenomenex Synergi C18 150×25 mm 10 μm; Mobile phase: A for H₂O (0.1% TFA) and B for Acetonitrile; Gradient: B 38%-68% in 10 min linearly; Flow rate: 25 mL/min; Column temperature: R.T.; Wavelength: 220 nm and 254 nm) to provide the desired tert-butyl 3-(7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3,8-diazabicyclo[3.2.1]octane-8-carboxylate (25 mg, 0.037 mmol, 27.6% yield) as a yellow solid, which was used in the next step directly. MS (ESI) m/z: 680.2 [M+H]⁺.

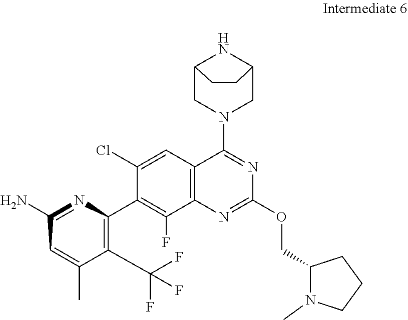

6-(6-chloro-4-{3,8-diazabicyclo[3.2.1]octan-3-yl}-8-fluoro-2-{[(2S)-1-methylpyrrolidin-2-yl]methoxy}quinazolin-7-yl)-4-methyl-5-(trifluoromethyl)pyridin-2-amine

To a solution of tert-butyl 3-(7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3,8-diazabicyclo[3.2.1]octane-8-carboxylate (Intermediate 6K, 20 mg, 0.03 mmol) in DCM (1.5 mL) was added TFA (0.5 mL, 6.53 mmol) at 25° C. The reaction mixture was stirred at 25° C. for 3 hours. The reaction mixture was filtered and the filtrate was collected. The filtrate was concentrated under reduced pressure. The residue was purified by preparation HPLC (FA as additive, Instrument: GX-p; Column: Phenomenex Synergi C18 150×25 mm, 10 μm; Mobile phase: A for H₂O (0.225% FA) and B for Acetonitrile; Gradient: B 3%-33% in 10 min linearly; Flow rate: 15 mL/min; Column temperature: R.T.; Wavelength: 220 nm, 254 nm) to provide the desired product. The mixture of atropisomers was separated by chiral SFC (Column: Cellucoat 50×4.6 mm I.D., 3 μm Mobile phase: Phase A for CO₂, and Phase B for IPA (0.05% DEA); Gradient elution: 40% IPA (0.05% DEA) in CO₂ Flow rate: 3 mL/min; Detector: PDA; Column Temp: 35° C.; Back Pressure: 100 Bar) to obtain two products peaks. The peak 1 product: Intermediate 6 (4.88 mg, 0.0082 mmol, 27.9% yield). MS (ESI) m/z 580.0 [M+1]⁺; ¹H NMR (400 MHz, METHANOL-d₄) δ 7.89 (s, 1H), 6.62 (s, 1H), 4.77 (br dd, J=13.6, 2.8 Hz, 1H), 4.63-4.52 (m, 3H), 3.95-3.87 (m, 2H), 3.76-3.69 (m, 2H), 3.64-3.48 (m, 2H), 3.16-3.02 (m, 1H), 2.96 (s, 3H), 2.45 (d, J=1.2 Hz, 3H), 2.39-2.28 (m, 1H), 2.13-1.93 (m, 7H).

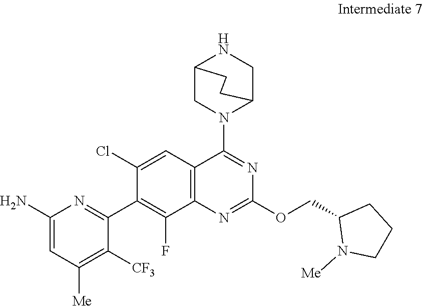 [SMILES: Cc1cc(N)nc(-c2c(Cl)cc3c(N4CC5CCC4CN5)nc(OC[C@@H]4CCCN4C)nc3c2F)c1C(F)(F)F]

### 6-(6-chloro-4-{2,5-diazabicyclo[2.2.2]octan-2-yl}-8-fluoro-2-{[(2S)-1-methylpyrrolidin-2-yl]methoxy}quinazolin-7-yl)-4-methyl-5-(trifluoromethyl)pyridin-2-amine

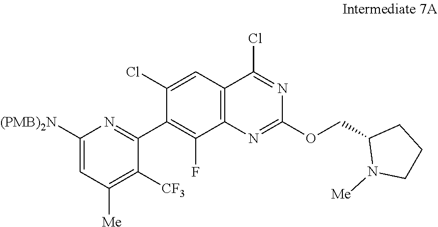

### 6-(4,6-dichloro-8-fluoro-2-(((S)-1-methylpyrrolidin-2-yl)methoxy)quinazolin-7-yl)-N,N-bis(4-methoxybenzyl)-4-methyl-5-(trifluoromethyl)pyridin-2-amine

To a solution of 7-(6-(bis(4-methoxybenzyl)amino)-4-methyl-3-(trifluoromethyl)-pyridin-2-yl)-6-chloro-8-fluoro-2-(((S)-1-methylpyrrolidin-2-yl)methoxy)-quinazolin-4(3H)-one (Intermediate 61, 50 mg, 0.07 mmol) in POCl₃ (1.5 mL, 16.09 mmol) was added DIEA (0.01 mL, 0.07 mmol). The reaction mixture was stirred at 50° C. for 3 hours. The reaction mixture was concentrated under reduced pressure and treated with ethyl acetate (10 mL). The mixture was poured into water (20 mL) and extracted with ethyl acetate (10 mL×2). The combined organic layers were washed with saturated NaHCO₃ solution (20 mL) and brine (20 mL), dried over anhydrous sodium sulfate, filtered and concentrated under reduced pressure to provide the crude product 6-(4,6-dichloro-8-fluoro-2-(((S)-1-methylpyrrolidin-2-yl)methoxy)quinazolin-7-yl)-N,N-bis(4-methoxybenzyl)-4-methyl-5-(trifluoromethyl)pyridin-2-amine (50 mg, 0.067 mmol, 97.5% yield) as a yellow solid. MS (ESI) m/z: 744.0 [M+H]⁺.

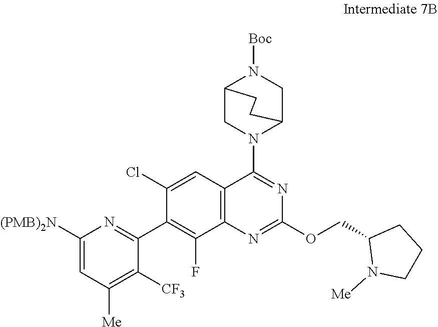

### tert-butyl 5-(7-(6-(bis(4-methoxybenzyl)amino)-4-methyl-3-(trifluoromethyl)pyridin-2-yl)-6-chloro-8-fluoro-2-(((S)-1-methylpyrrolidin-2-yl)methoxy)quinazolin-4-yl)-2,5-diazabicyclo[2.2.2]octane-2-carboxylate

To a solution of 6-(4,6-dichloro-8-fluoro-2-(((S)-1-methylpyrrolidin-2-yl)methoxy)-quinazolin-7-yl)-N,N-bis(4-methoxybenzyl)-4-methyl-5-(trifluoromethyl)pyridin-2-amine (Intermediate 7A, 40 mg, 0.05 mmol) in DMA (2 mL) were added DIEA (0.02 mL, 0.2700 mmol) and tert-butyl 2,5-diazabicyclo[2.2.2]octane-2-carboxylate (31.7 mg, 0.15 mmol). The mixture was stirred at 50° C. for 12 hours. The reaction mixture was diluted with water (10 mL), and extracted with ethyl acetate (10 mL×3). The combined organic layers were washed with brine (20 mL×3), dried over anhydrous sodium sulfate, filtered and concentrated under reduced pressure. The residue was purified by preparation TLC (silica gel plate, dichloromethane:methanol=10:1) to provide the product (40 mg, 0.044 mmol, 80.9% yield) as a colorless oil. MS (ESI) m/z: 920.3 [M+H]⁺.

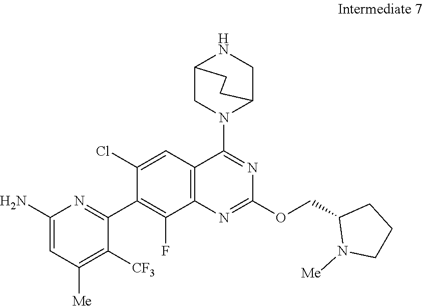 [SMILES: Cc1cc(N)nc(-c2c(Cl)cc3c(N4CC5CCC4CN5)nc(OC[C@@H]4CCCN4C)nc3c2F)c1C(F)(F)F]

### 6-(4,6-dichloro-8-fluoro-2-(((S)-1-methylpyrrolidin-2-yl)methoxy)quinazolin-7-yl)-N,N-bis(4-methoxybenzyl)-4-methyl-5-(trifluoromethyl)pyridin-2-amine

A solution of tert-butyl 5-(7-(6-(bis(4-methoxybenzyl)amino)-4-methyl-3-(trifluoromethyl)pyridin-2-yl)-6-chloro-8-fluoro-2-(((S)-1-methylpyrrolidin-2-yl)methoxy)-quinazolin-4-yl)-2,5-diazabicyclo[2.2.2]octane-2-carboxylate (Intermediate 7B, 40 mg, 0.040 mmol) in TFA (4 mL, 52.24 mmol) was stirred at 50° C. for 4 hours. The mixture was then concentrated to dryness. The residue was purified by preparative HPLC (formic acid as additive, Instrument: ACSWH-GX-Q; Column: Shim-pack C18 150×25, 10 μm; Mobile phase: A for H₂O (0.225% FA) and B for Acetonitrile; Gradient: B 2%-35% in 11 min linearly; Flow rate: 25 mL/m; Column temperature: R.T.; Wavelength: 220 nm. 254 nm) to provide the desired product (11.1 mg, 0.019 mmol, 44.2% yield) as a white solid. MS (ESI) m/z: 580.2 [M+H]; ¹H NMR (400 MHz, CD₃OD) δ 8.53 (s, 0.4H), 8.05 (s, 1H), 6.61 (s, 1H), 4.95 (br s, 1H), 4.68-4.49 (m, 2H), 4.35 (d, J=11.6 Hz, 1H), 4.27-4.19 (m, 1H), 3.62-3.33 (m, 4H), 2.86-2.70 (m, 4H), 2.45 (d, J=1.2 Hz, 3H), 2.40-2.18 (m, 2H), 2.16-1.79 (m, 7H).

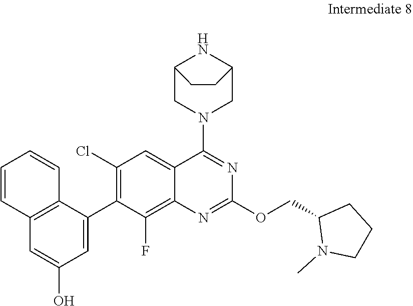 [SMILES: CN1CCC[C@H]1COc1nc(N2CC3CCC(C2)N3)c2cc(Cl)c(-c3cc(O)cc4ccccc34)c(F)c2n1]

### 4-(6-chloro-4-{3,8-diazabicyclo[3.2.1]octan-3-yl}-8-fluoro-2-{[(2S)-1-methylpyrrolidin-2-yl]methoxy}quinazolin-7-yl)naphthalen-2-ol

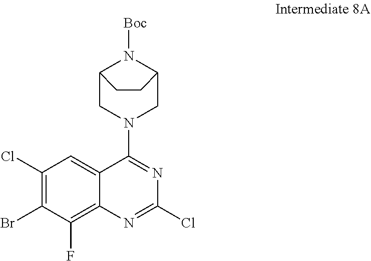 [SMILES: CC(C)(C)OC(=O)N1C2CCC1CN(c1nc(Cl)nc3c(F)c(Br)c(Cl)cc13)C2]

### tert-butyl 3-(7-bromo-2,6-dichloro-8-fluoroquinazolin-4-yl)-3,8-diazabicyclo[3.2.1]octane-8-carboxylate

To a solution of 7-bromo-2,4,6-trichloro-8-fluoroquinazoline (300 mg, 3.03 mmol) in dioxane (8 mL) was added DIPEA (0.476 mL, 2.72 mmol) and tert-butyl 3,8-diazabicyclo[3.2.1]octane-8-carboxylate (193 mg, 0.908 mmol). The resulting mixture was stirred at 25° C. for 2 hours. LCMS showed the reaction was completed. The mixture was concentrated. The residue was diluted with ethyl acetate (50 mL) and washed with water (30 mL×2) and brine (50 mL). The organic phase was dried over anhydrous sodium sulphate, filtered and concentrated under reduced pressure. The residue was purified by silica gel column chromatography (12 g ISCO column, MeOH/DCM, 0-5%, 20 min.) to provide tert-butyl 4-(7-bromo-2,6-dichloro-8-fluoroquinazolin-4-yl)piperazine-1-carboxylate (415 mg, 0.82 mmol, 90% yield) as a white solid. MS (ESI) m/z 507.0 [M+1]⁺. ¹H NMR (499 MHz, DMSO-d₆) δ 8.10 (d, J=1.9 Hz, 1H), 4.38 (br d, J=10.6 Hz, 2H), 4.25 (br s, 2H), 3.66 (m, 2H) 1.79 (m, 2H), 1.62 (m, 2H), 1.47 s, 9H).

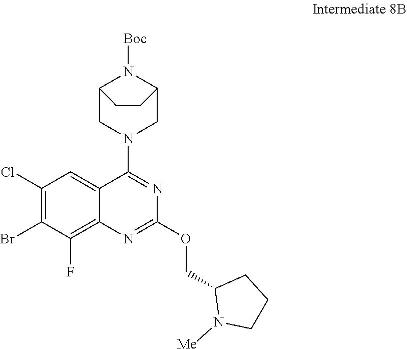 [SMILES: CN1CCC[C@H]1COc1nc(N2CC3CCC(C2)N3C(=O)OC(C)(C)C)c2cc(Cl)c(Br)c(F)c2n1]

### tert-butyl-3-(7-bromo-6-chloro-8-fluoro-2-(((S)-1-methylpyrrolidin-2-yl)methoxy)quinazolin-4-yl)-3,8-diazabicyclo[3.2.1]octane-8-carboxylate

To a solution of tert-butyl 3-(7-bromo-2,6-dichloro-8-fluoroquinazolin-4-yl)-3,8-diazabicyclo[3.2.1]octane-8-carboxylate (Intermediate 8A, 415 mg, 0.820 mmol) in DMSO (6 mL) was added cesium fluoride (187 mg, 1.230 mmol), and (S)-(1-methylpyrrolidin-2-yl)methanol (236 mg, 2.050 mmol). The mixture was heated to 100° C. for 2 hours. The residue was diluted with DCM (50 mL) and washed with water (30 mL×2) and brine (50 mL). The organic phase was dried over anhydrous sodium sulphate, filtered and concentrated under reduced pressure. The residue was purified by silica gel column chromatography (12 g ISCO column, MeOH/DCM, 0-15%, 30 min.) to provide tert-butyl 3-(7-bromo-6-chloro-8-fluoro-2-{[(2S)-1-methylpyrrolidin-2-yl]methoxy}quinazolin-4-yl)-3,8-diazabicyclo[3.2.1]octane-8-carboxylate (108 mg, 0.185 mmol, 22.5% yield) as a yellow oil. MS (ESI) m/z 586.1 [M+1]⁺.

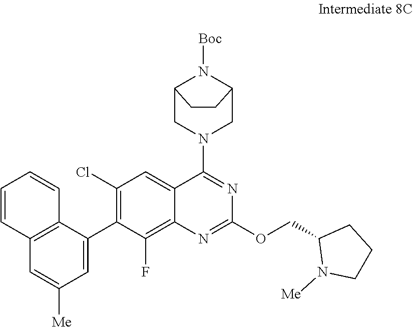 [SMILES: Cc1cc(-c2c(Cl)cc3c(N4CC5CCC(C4)N5C(=O)OC(C)(C)C)nc(OC[C@@H]4CCCN4C)nc3c2F)c2ccccc2c1]

### tert-butyl 3-[6-chloro-8-fluoro-7-(3-hydroxynaphthalen-1-yl)-2-{[(2S)-1-methylpyrrolidin-2-yl]methoxy}quinazolin-4-yl]-3,8-diazabicyclo[3.2.1]octane-8-carboxylate

A suspension of tert-butyl 3-(7-bromo-6-chloro-8-fluoro-2-{[(2S)-1-methylpyrrolidin-2-yl]methoxy}quinazolin-4-yl)-3,8-diazabicyclo[3.2.1]octane-8-carboxylate (Intermediate 8B, 108 mg, 0.185 mmol) and Na₂CO₃ in 1,4-dioxane (2954 μl) and water (739 μl) was degassed and Pd(Ph₃)₄ (42.7 mg, 0.037 mmol) was added in one portion. The mixture was degassed again and heated in a pressure vial at 95° C. for 1 hour. The reaction mixture was diluted with water (10 mL) and DCM (10 mL). The mixture was extracted with DCM (10 mL×2). The combined organic layers were washed with brine (20 mL), dried over anhydrous sodium sulfate, filtered and concentrated under reduced pressure to provide a crude tert-butyl 3-[6-chloro-8-fluoro-7-(3-hydroxynaphthalen-1-yl)-2-{[(2S)-1-methylpyrrolidin-2-yl]methoxy}quinazolin-4-yl]-3,8-diazabicyclo[3.2.1]octane-8-carboxylate, MS (ESI) m/z 857.2 [M+1]⁺, which was used directly in the next step.

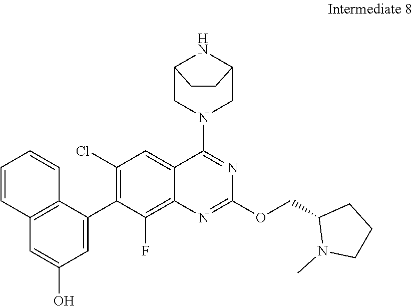 [SMILES: CN1CCC[C@H]1COc1nc(N2CC3CCC(C2)N3)c2cc(Cl)c(-c3cc(O)cc4ccccc34)c(F)c2n1]

### 4-(6-chloro-4-{3,8-diazabicyclo[3.2.1]octan-3-yl}-8-fluoro-2-{[(2S)-1-methylpyrrolidin-2-yl]methoxy}quinazolin-7-yl)naphthalen-2-ol

To a solution of the crude tert-butyl 3-[6-chloro-8-fluoro-7-(3-hydroxynaphthalen-1-yl)-2-{[(2S)-1-methylpyrrolidin-2-yl]methoxy}quinazolin-4-yl]-3,8-diazabicyclo[3.2.1]octane-8-carboxylate in DCM (4 mL) was added TFA (0.8 mL) at 25° C. The reaction mixture was stirred at 25° C. for 1 hour. The reaction mixture was concentrated under reduced pressure to provide a residue, which was purified via preparative HPLC with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 5% B, 5-45% B over 20 minutes, then a 0-minute hold at 100% B; Flow Rate: 20 mL/min; Column Temperature: 25 C. Fractions containing the desired product were combined and dried via centrifugal evaporation. The material was further purified via preparative HPLC with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 4% B, 4-44% B over 20 minutes, then a 0-minute hold at 100% B; Flow Rate: 20 mL/min; Column Temperature: 25 C. Fractions containing the desired product were combined and dried via centrifugal evaporation to provide the desired product (22.3 mg, 0.039 mmol) as a white solid. MS (ESI) m/z 547.93 [M+1]⁺; ¹H NMR (500 MHz, DMSO-d₆) δ 8.02 (s, 1H), 7.82 (br d, J=8.2 Hz, 1H), 7.46 (br t, J=7.5 Hz, 1H), 7.36-7.15 (m, 3H), 7.24 (m, 1H), 4.63 (br d, 1H), 4.60 (m, 3H), 4.21 (br s, 2H), 3.83 (m, 2H), 2.97 (m, 2H), 2.55 (m, 4H), 2.28 (s, 1H), 2.08 (m, 2H), 1.97 (m, 5H).

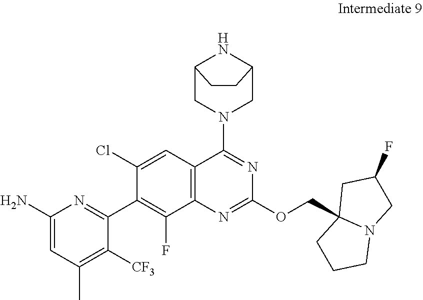 [SMILES: Cc1cc(N)nc(-c2c(Cl)cc3c(N4CC5CCC(C4)N5)nc(OC[C@]45CCCN4C[C@H](F)C5)nc3c2F)c1C(F)(F)F]

### 6-(2-{[(2R,7aR)-2-fluoro-hexahydro-1H-pyrrolizin-7a-yl]methoxy}-6-chloro-4-{3,8-diazabicyclo[3.2.1]octan-3-yl}-8-fluoroquinazolin-7-yl)-4-methyl-5-(trifluoromethyl)pyridin-2-amine

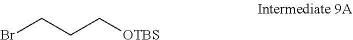 [SMILES: CC(C)(C)[Si](C)(C)OCCCBr]

### (3-bromopropoxy)(tert-butyl)dimethylsilane

To a stirred solution of 3-bromopropan-1-ol (10.00 g, 71.9 mmol) in anhydrous dichloromethane (50 mL) under nitrogen at 0° C. were added imidazole (4.90 g, 71.9 mmol) followed by tert-butyldimethylsilyl chloride (14.10 g, 94 mmol). Reaction mixture was slowly warmed to room temperature and stirred at the same temperature for 12 h. Reaction mixture was quenched with saturated aq. ammonium chloride solution (20 mL) and diluted with water (50 mL). The suspension was extracted with dichloromethane (3×200 mL). Combined organic layers were dried over anhydrous sodium sulphate, filtered, and concentrated reduced pressure to obtain the crude residue of the product. The residue was purified by silica gel column chromatography with 0-5% of ethyl acetate in petroleum ether to obtain the (3-bromopropoxy)(tert-butyl)dimethylsilane (8.00 g, 31.6 mmol, 43.9% yield) as colorless oil. ¹H NMR (400 MHz, CD₃OD): δ 3.69 (t, J=4.0 Hz, 2H), 3.57 (t, J=6.4 Hz, 2H), 1.99-1.93 (m, 2H), 0.87 (s, 9H), 0.06 (s, 6H) ppm.

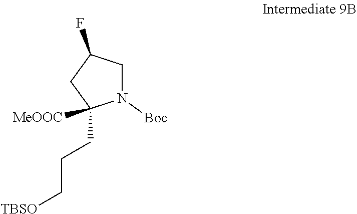 [SMILES: COC(=O)[C@@]1(CCCO[Si](C)(C)C(C)(C)C)C[C@@H](F)CN1C(=O)OC(C)(C)C]

### 1-(tert-butyl) 2-methyl (2R,4R)-2-(3-((tert-butyldimethylsilyl)oxy)propyl)-4-fluoropyrrolidine-1,2-dicarboxylate

To a stirred solution of 1-(tert-butyl) 2-methyl (2S,4R)-4-fluoropyrrolidine-1,2-dicarboxylate (Intermediate 9A, 5.00 g, 20.22 mmol) in anhydrous THE (30 mL) under nitrogen atmosphere at −25° C. was added LiHMDS (1M in THF, 30.3 mL, 30.3 mmol) dropwise. Reaction mixture was stirred at same temperature for 30 min prior dropwise addition of (3-bromopropoxy)(tert-butyl)dimethylsilane (7.68 g, 30.3 mmol). The reaction mixture was stirred at the same temperature for 30 min and then slowly warmed to room temperature. Reaction mixture was quenched with saturated aq. ammonium chloride solution (15 mL) and then diluted with water (50 mL). The mixture was extracted with ethyl acetate (3×100 mL). The combined organic layers were dried over anhydrous Na₂SO₄, filtered and concentrated reduced pressure. The residue was purified by silica gel column chromatography with 20-30% of ethyl acetate in petroleum ether to obtain the 1-(tert-butyl) 2-methyl (2R,4R)-2-(3-((tert-butyldimethylsilyl)oxy)propyl)-4-fluoropyrrolidine-1,2-dicarboxylate (7.23 g, 17.24 mmol, 85.0% yield) as colorless oil. ¹H NMR (400 MHz, CDCl₃): δ 5.18-5.03 (m, 1H), 4.10-3.98 (m, 1H), 3.78 (s, 3H), 3.74-3.52 (m, 3H), 2.48-2.23 (m, 3H), 2.19-1.98 (m, 1H), 1.65 (s, 9H), 1.48-1.44 (m, 2H), 0.90 (s, 9H), 0.60 (s, 6H) ppm. ¹⁹F (376 MHz, CD₃OD): δ −172.47 to −172.96 (m) ppm. LCMS-ELSD (ESI) m/z: 320.2 [M+H−Boc]⁺.

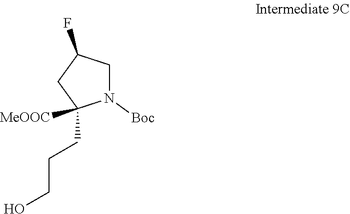 [SMILES: COC(=O)[C@@]1(CCCO)C[C@@H](F)CN1C(=O)OC(C)(C)C]

### 1-(tert-butyl) 2-methyl (2R,4R)-4-fluoro-2-(3-hydroxypropyl)pyrrolidine-1,2-dicarboxylate

To a stirred solution of 1-(tert-butyl) 2-methyl (2R,4R)-2-(3-((tert-butyldimethylsilyl)oxy)propyl)-4-fluoropyrrolidine-1,2-dicarboxylate (Intermediate 9B, 7.00 g, 16.68 mmol) at 25° C. was added TBAF (1M in THF, 16.68 mL, 16.68 mmol) dropwise, and stirred at the same temperature for 4 h. Reaction mixture was quenched with saturated aq. ammonium chloride solution (90 mL) and diluted with ethyl acetate (100 mL). The mixture was extracted with ethyl acetate (3×150 mL). Combined organic layer was dried over anhydrous sodium sulphate, filtered, and concentrated reduced pressure to obtain the crude residue as colorless oil. The crude was purified by silica gel column chromatography using 50% ethyl acetate in petroleum ether to obtain 1-(tert-butyl) 2-methyl (2R,4R)-4-fluoro-2-(3-hydroxypropyl)pyrrolidine-1,2-dicarboxylate (4.97 g, 15.64 mmol, 94.0% yield) as colorless oil. LCMS-ELSD (ESI) m/z: 206.2 [M+H-Boc]⁺.

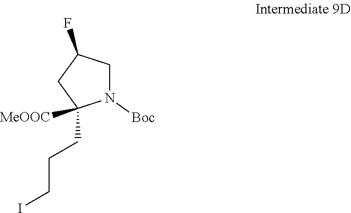 [SMILES: COC(=O)[C@@]1(CCCI)C[C@@H](F)CN1C(=O)OC(C)(C)C]

### 1-(tert-butyl) 2-methyl (2R,4R)-4-fluoro-2-(3-iodopropyl)pyrrolidine-1,2-dicarboxylate

To a stirred solution of 1-(tert-butyl) 2-methyl (2R,4R)-4-fluoro-2-(3-hydroxypropyl)pyrrolidine-1,2-dicarboxylate (Intermediate 9C, 9.00 g, 29.5 mmol) in dichloromethane (100 mL) at 0° C. was added triphenylphosphine (15.50 g, 58.9 mmol), imidazole (6.02 g, 88 mmol) and the reaction mixture was stirred at same temperature for 10 min before addition of iodine (29.9 g, 118 mmol). The reaction mixture was stirred at room temperature for 12 h. The reaction mixture was then quenched with sat. aq. sodium thiosulfate solution (30 mL) and the suspension was extracted with dichloromethane (2×200 mL). The combined organic layers were dried over anhydrous sodium sulphate, filtered, and concentrated reduced pressure to obtain the crude residue. This crude was purified by silica gel column chromatography using 10-20% ethyl acetate in petroleum ether to obtain 1-(tert-butyl) 2-methyl (2R,4R)-4-fluoro-2-(3-iodopropyl)pyrrolidine-1,2-dicarboxylate (10.10 g, 24.32 mmol, 83.0% yield) as colourless oil. LCMS-ELSD (ESI) m/z: 361.2 [M+H-Boc]⁺.

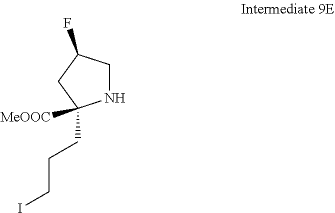 [SMILES: COC(=O)[C@@]1(CCCI)C[C@@H](F)CN1]

### tert-butyl 3-(6-chloro-8-fluoro-7-(3-hydroxynaphthalen-1-yl)-2-(((R)-1-(piperidin-1-yl)propan-2-yl)oxy)quinazolin-4-yl)-3,8-diazabicyclo[3.2.1]octane-8-carboxylate

To a stirred solution of 1-(tert-butyl) 2-methyl (2R,4R)-4-fluoro-2-(3-iodopropyl)pyrrolidine-1,2-dicarboxylate (Intermediate 9D, 10.0 g, 24.08 mmol) in dichloromethane (20 mL) at 25° C. was added HCl in dioxane (8.03 mL, 24.08 mmol) and reaction mixture was stirred for 6 h at same temperature. On complete consumption of starting material, the reaction mixture was concentrated reduced pressure at room temperature to obtain the crude residue of methyl (2R,4R)-4-fluoro-2-(3-iodopropyl)pyrrolidine-2-carboxylate HCl (6.5 g, 20.63 mmol, 86.0% yield), which was used as such for the next step without any further purification. LCMS-ELSD (ESI) m/z: 188.2 [M+H]⁺.

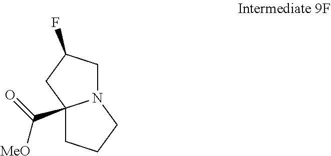 [SMILES: COC(=O)[C@]12CCCN1C[C@H](F)C2]

### methyl (2R,7aR)-2-fluorotetrahydro-1H-pyrrolizine-7a(5H)-carboxylate

To a stirred solution of methyl (2R,4R)-4-fluoro-2-(3-iodopropyl)pyrrolidine-2-carboxylate (Intermediate 9E, 6.5 g, 20.63 mmol) in anhydrous acetonitrile (30 mL) was added triethylamine (10 mL) at room temperature. Reaction mixture was stirred at 45° C. for 12 h. Reaction mixture was concentrated reduced pressure to obtain the crude residue. The residue was purified by column chromatography (alumina-neutral) using 40-50% ethyl acetate in petroleum ether to obtain the methyl (2R,7aR)-2-fluorotetrahydro-1H-pyrrolizine-7a(5H)-carboxylate (3.20 g, 83.0% yield) as pale-yellow oil. ¹H NMR (400 MHz, DMSO-d₆): δ 5.76-5.19 (m, 1H), 3.59 (s, 3H), 3.25-3.16 (m, 1H), 2.97-2.95 (m, 1H), 2.86-2.76 (m, 1H), 2.68-2.51 (m, 2H), 2.02-1.91 (m, 2H), 1.85-1.72 (m, 3H) ppm. LCMS-ELSD (ESI) m/z: 188.3 [M+H]⁺.

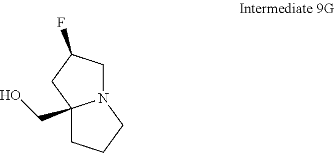 [SMILES: OC[C@]12CCCN1C[C@H](F)C2]

### ((2R,7aR)-2-fluorotetrahydro-1H-pyrrolizin-7a(5H)-yl)methanol

To a stirred solution of methyl (2R,7aR)-2-fluorotetrahydro-1H-pyrrolizine-7a(5H)-carboxylate (Intermediate 9F, 3.20 g, 17.09 mmol) under nitrogen at 0° C. was added LiAlH₄ (2M in THF, 17.09 mL, 34.2 mmol) dropwise over 10 min. After addition, the reaction mixture was warmed to room temperature in 30 min. The mixture was stirred at this temperature for 1 h prior to being quenched with saturated aq. ammonium chloride (5 mL) at 0° C. Once the effervescence was stopped, anhydrous sodium sulphate was added to the reaction mixture, followed by dichloromethane (20 mL). The reaction mixture was stirred for 20 min and filtered. The filtrate was collected, dried over anhydrous sodium sulphate, filtered, and concentrated reduced pressure to obtain the crude residue of ((2R,7aR)-2-fluorotetrahydro-1H-pyrrolizin-7a(5H)-yl)methanol (2.20 g, 13.68 mmol, 80.0% yield) as pale brown oil. ¹H NMR (400 MHz, CD₃OD): δ 5.34-5.10 (m, 1H), 3.51-3.29 (m, 3H), 3.25-3.10 (m, 1H), 3.04-2.93 (m, 1H), 2.90-2.73 (m, 1H), 2.71-2.59 (m, 1H), 2.28-2.12 (m, 1H), 1.95-1.73 (m, 4H), 1.68-1.66 (m, 1H) ppm. ¹⁹F (376 MHz, CD₃OD): δ −175.69 to −176.04 (m) ppm. LCMS-ELSD (ESI) m/z: 160.0 [M+H]⁺.

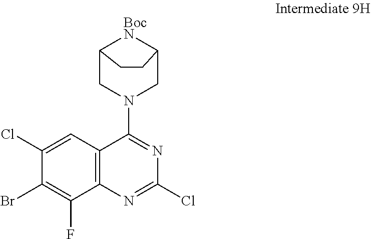 [SMILES: CC(C)(C)OC(=O)N1C2CCC1CN(c1nc(Cl)nc3c(F)c(Br)c(Cl)cc13)C2]

### tert-butyl 3-(7-bromo-2,6-dichloro-8-fluoroquinazolin-4-yl)-3,8-diazabicyclo[3.2.1]octane-8-carboxylate

To a solution of 7-bromo-2,4,6-trichloro-8-fluoroquinazoline (3.5 g, 10.6 mmol) and tert-butyl 3,8-diazabicyclo[3.2.1]octane-8-carboxylate (2240 mg, 10.55 mmol) in THE (50 mL) was added DIEA (2.48 mL, 26.5 mmol). The mixture was stirred at 20° C. for 12 h. LCMS showed the reaction was completed and desired product was detected. The reaction mixture was diluted with EtOAc (40 mL) and water (40 mL). The mixture was extracted with EtOAc (40 mL×3). The combined organic layers were washed with brine (100 mL), dried over anhydrous Na₂SO₄, filtered and concentrated in vacuum. The crude product tert-butyl 3-(7-bromo-2,6-dichloro-8-fluoroquinazolin-4-yl)-3,8-diazabicyclo[3.2.1]octane-8-carboxylate (5.3 g, 10.5 mmol, 98.8% yield) was used in the next step without purification. MS (ESI) m/z: 506.9 [M+3]⁺. ¹H NMR (400 MHz, CDCl₃) δ 7.75 (d, J=2.0 Hz, 1H), 4.40 (s, 4H), 3.78-3.51 (m, 2H), 2.02-1.90 (m, 2H), 1.77-1.67 (m, 2H), 1.53 (s, 9H).

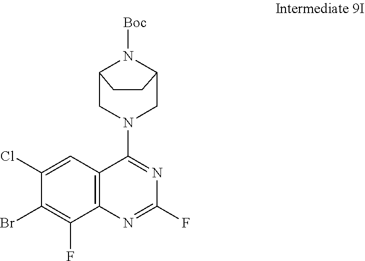 [SMILES: CC(C)(C)OC(=O)N1C2CCC1CN(c1nc(F)nc3c(F)c(Br)c(Cl)cc13)C2]

### tert-butyl (1R,5S)-3-(7-bromo-6-chloro-2,8-difluoroquinazolin-4-yl)-3,8-diazabicyclo[3.2.1]octane-8-carboxylate

To a degassed solution of tert-butyl 3-(7-bromo-2,6-dichloro-8-fluoroquinazolin-4-yl)-3,8-diazabicyclo[3.2.1]octane-8-carboxylate (Intermediate 9H, 1 g, 3.03 mmol) in DMA (80 mL) was added cesium fluoride (5.25 g, 34.6 mmol). This was degassed with nitrogen gas for 10 min and was heated at 88° C. for 5 h in a sealed tube. Water (200 mL) and ethyl acetate (150 mL) were added and this was stirred for 15 min. The separated aqueous layer was extracted with ethyl acetate (2×100 mL) and the combined organic layers were dried over anhydrous Na₂SO₄, filtered, and concentrated. The resulting residue was purified by flash column chromatography using 15-25% ethyl acetate in petroleum ether as an eluent to provide tert-butyl 3-(7-bromo-6-chloro-2,8-difluoroquinazolin-4-yl)-3,8-diazabicyclo[3.2.1]octane-8-carboxylate (4.7 g, 8.77 mmol, 63.4% yield) as a pale yellow solid. MS (ESI) m/z 489.0 [M+1]⁺.

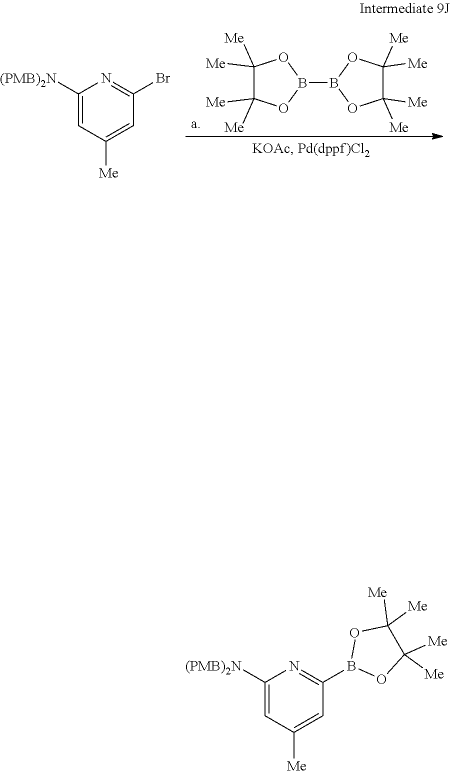

### N,N-bis(4-methoxybenzyl)-4-methyl-6-(4,4,5,5-tetramethyl-1,3,2-dioxaborolan-2-yl)pyridin-2-amine

6-bromo-N,N-bis(4-methoxybenzyl)-4-methylpyridin-2-amine (20 g, 46.8 mmol), bis(pinacolato)diboron (35.7 g, 140 mmol), and potassium acetate (18.37 g, 187 mmol) were combined as solids and dissolved in dioxane (200 mL). Nitrogen was sparged through the solution for 20 min and 1,1′-bis(diphenylphosphino)ferrocene-palladium(II)dichloride dichloromethane complex (1.9 g, 2.3 mmol) was added, The reaction mixture was heated at 100° C. for 5 h. The reaction mixture was diluted with EtOAc (200 mL) and was filtered through diatomaceous earth (Celite™). The filtrate was concentrated to provide the desired product (22 g, 40.7 mmol, 87% yield).

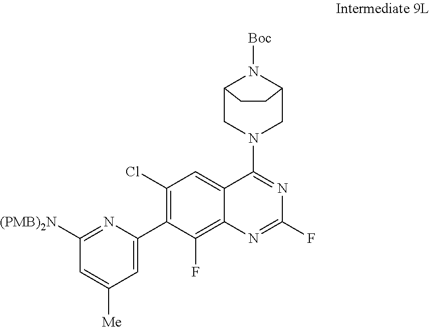

### tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate

To a degassed solution of Intermediate 91 (2.00 g, 4.08 mmol) in anhydrous 1,4-dioxane (20 mL) were added potassium phosphate (1.73 g, 8.17 mmol), N,N-bis(4-methoxybenzyl)-4-methyl-6-(4,4,5,5-tetramethyl-1,3,2-dioxaborolan-2-yl)pyridin-2-amine (Intermediate 9J, 5.8 g, 12.25 mmol), PdCl₂(dppf) (149 mg, 0.204 mmol). The mixture was degassed again and heated at 80° C. for 48 h. Reaction progress was monitored by LCMS. The reaction vessel was allowed to cool to ambient temperature, diluted with ethyl acetate (40 mL), filtered through a bed of diatomaceous earth (Celite™) and concentrated in vacuo to provide crude product. The residue was purified by silica gel column chromatography using 30% ethyl acetate in petroleum ether to obtain tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate (1.5 g, 1.74 mmol, 42% yield). ¹H NMR (400 MHz, CDCl₃): δ 7.76 (d, J=1.6 Hz, 1H), 7.20-7.18 (d, J=8.8 Hz, 4H), 6.86 (dt, J=9.6 Hz, 4H), 6.60 (s, 1H), 6.38 (s, 1H), 4.60 (s, 3H), 4.39-4.21 (m, 4H), 3.63 (s, 6H), 2.29 (s, 3H), 1.98-1.96 (m, 6H), 1.76-1.63 (m, 2H), 1.49 (s, 9H) ppm. LCMS (ESI) m/z: 757.2 [M+H]⁺.

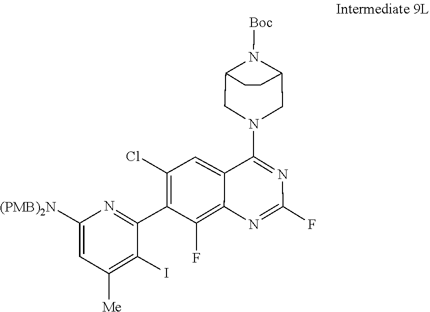

### tert-butyl 3-(7-(6-(bis(4-methoxybenzyl)amino)-3-iodo-4-methylpyridin-2-yl)-6-chloro-2,8-difluoroquinazolin-4-yl)-3,8 diazabicyclo[3.2.1]octane-8-carboxylate

To a stirred solution of tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate (Intermediate 9K, 1.40 g, 1.849 mmol) in anhydrous acetonitrile (15 mL) under nitrogen at 0° C. were added N-iodosuccinimide (0.42 g, 1.849 mmol) and trifluoroacetic acid (0.028 mL, 0.370 mmol). The reaction mixture was allowed to reach room temperature over one hour. The reaction mixture was then quenched with saturated aq. sodium thiosulphate (5 mL) and saturated aq. sodium bicarbonate (4 mL). The mixture was extracted with ethyl acetate (3×20 mL). Combined organic layers were dried over anhydrous sodium sulphate, filtered, and concentrated reduced pressure to obtain the crude residue. The crude residue was purified by silica gel column chromatography using 30% ethyl acetate in petroleum ether to obtain tert-butyl 3-(7-(6-(bis(4-methoxybenzyl)amino)-3-iodo-4-methylpyridin-2-yl)-6-chloro-2,8-difluoroquinazolin-4-yl)-3,8-diazabicyclo[3.2.1]octane-8-carboxylate (1.42 g, 1.560 mmol, 84% yield) as pale yellow fluffy solid. LCMS (ESI) m/z: 883.3 [M+H]⁺.

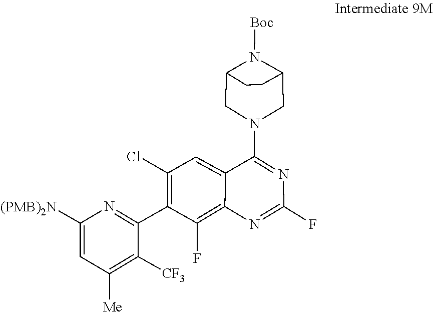

### tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate

To a stirred solution of tert-butyl 3-(7-(6-(bis(4-methoxybenzyl)amino)-3-iodo-4-methylpyridin-2-yl)-6-chloro-2,8-difluoroquinazolin-4-yl)-3,8-diazabicyclo[3.2.1]octane-8-carboxylate (Intermediate 9L, 1.40 g, 1.585 mmol) in anhydrous DMA (10 mL) in a sealed tube under nitrogen atmosphere was added copper(I) iodide (0.60 g, 3.17 mmol). The reaction mixture was degassed 10 min before the addition of methyl 2,2-difluoro-2-(fluorosulfonyl)acetate (0.91 g, 4.76 mmol) and reaction mixture was heated to 90° C. for 12 h. Reaction progress was monitored by LCMS. Reaction mixture was diluted with diethyl ether (20 mL) and water (10 mL). Layers were separated and aqueous layer was extracted with diethyl ether (3×20 mL). Combined organic layers were dried over anhydrous sodium sulphate, filtered, and concentrated reduced pressure to obtain the crude residue. The crude was purified by silica gel column chromatography using 30% ethyl acetate in petroleum ether to obtain tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (0.85 g, 0.630 mmol, 40% yield) as a pale yellow solid. ¹H NMR (400 MHz, CDCl₃): δ 7.77 (s, 1H), 7.16 (d, J=8.8 Hz, 4H), 6.87 (dt, J=9.6 and 2.8 Hz, 4H), 6.43 (s, 1H), 4.76-4.72 (m, 2H), 4.59-4.55 (m, 2H), 3.81 (s, 6H), 2.43 (s, 3H), 1.97-1.82 (m, 4H), 1.97-1.82 (m, 4H), 1.53 (s, 9H) ppm. LCMS (ESI) m/z: 825.2 [M+H]⁺.

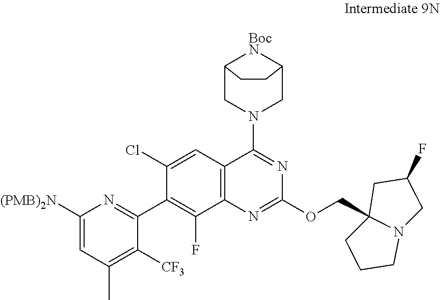

### tert-butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,8-diazabicyclo[3.2.1]octane-8-carboxylate

To a stirred solution of ((2R,7aR)-2-fluorotetrahydro-1H-pyrrolizin-7a(5H)-yl)methanol (Intermediate 9G, 0.23 g, 1.454 mmol) in anhydrous THE (2.1 mL) at 0° C. was added sodium hydride (43.6 mg, 1.091 mmol) and the reaction mixture was stirred at the same temperature for 30 min. After 30 min tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (Intermediate 9M, 0.60 g, 0.727 mmol) dissolved in anhydrous THF (10 mL) was added to the reaction mixture dropwise while maintaining the temperature 0° C. The reaction mixture was stirred for the next 2 h while warming the reaction mixture to room temperature. The reaction mixture was quenched with saturated aq. ammonium chloride solution (1 mL) and diluted with ethyl acetate (5 mL). The mixture was extracted with ethyl acetate (3×5 mL). Combined organic layers were dried over anhydrous sodium sulphate, filtered, and concentrated under a vacuum to obtain the crude residue of the product. Crude residue was purified over neutral alumina using 30% ethyl acetate in petroleum ether to obtain tert-butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,8-diazabicyclo[3.2.1]octane-8-carboxylate (657 mg, 0.703 mmol, 97.0% yield) obtained as pale yellow solid. LCMS (ESI) m/z: 964.3 [M+H]⁺.

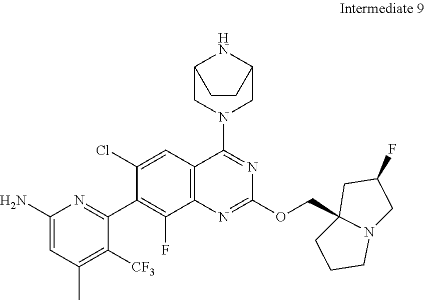 [SMILES: Cc1cc(N)nc(-c2c(Cl)cc3c(N4CC5CCC(C4)N5)nc(OC[C@]45CCCN4C[C@H](F)C5)nc3c2F)c1C(F)(F)F]

### 6-(2-{[(2R,7aR)-2-fluoro-hexahydro-1H-pyrrolizin-7a-yl]methoxy}-6-chloro-4-{3,8-diazabicyclo[3.2.1]octan-3-yl}-8-fluoroquinazolin-7-yl)-4-methyl-5-(trifluoromethyl)pyridin-2-amine

To a stirred solution of TFA (200 μl, 2.59 mmol) and triethyl silane (83 μl, 0.518 mmol) was added tert-butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,8-diazabicyclo[3.2.1]octane-8-carboxylate (Intermediate 9N, 0.50 g, 0.518 mmol) at room temperature. Then reaction mixture was stirred at 35° C. for 12 h. The progress of the reaction was monitored by LCMS. After completion, the reaction mixture was concentrated to remove most of the TFA under reduced pressure and below 35° C. The residue was co-evaporated with methanol (3×1 mL) to remove any residual TFA. The residue was neutralized with DIPEA and concentrated under reduced pressure to obtain a free base. The crude was then purified by silica gel (previously neutralized with DIPEA) column chromatography using a mixture of MeOH, dichloromethane and DIPEA (15:80:2.5) to obtain 6-(2-{[(2R,7aR)-2-fluoro-hexahydro-1H-pyrrolizin-7a-yl]methoxy}-6-chloro-4-{3,8-diazabicyclo[3.2.1]octan-3-yl}-8-fluoroquinazolin-7-yl)-4-methyl-5-(trifluoromethyl)pyridin-2-amine. Atropisomers were separated by chiral SFC (Column: OD 25×5 mm I.D., 5 μm. Mobile phase: Phase A for CO₂, and Phase B for MeOH (0.1% NH₄OH); Gradient elution: 40% MeOH (0.1% NH₄OH) in CO₂. Flow rate: 350 mL/min; Detector: UV 220 nm; Column Temp: 30° C.; Back Pressure: 100 Bar) to obtain two product peaks: peak 1 (Intermediate 9-1) and peak 2 (Intermediate 9-2).

Peak 1, isomer 1: Intermediate 9-1 (66 mg, 33% yield). MS (ESI) m/z 624.3 [M+1]⁺. ¹H NMR (499 MHz, DMSO-d₆) δ 7.88-7.76 (m, 1H), 6.94-6.75 (m, 2H), 6.57-6.42 (m, 1H), 5.49-5.24 (m, 1H), 4.36-4.27 (m, 1H), 4.22-4.16 (m, 1H), 4.11 (s, 1H), 4.07-4.01 (m, 1H), 3.58-3.49 (m, 3H), 3.48-3.42 (m, 1H), 3.01-2.92 (m, 1H), 2.89-2.74 (m, 1H), 2.58-2.54 (m, 1H), 2.40-2.36 (m, 3H), 2.35-2.26 (m, 1H), 1.87-1.87 (m, 1H), 1.96-1.78 (m, 3H), 1.71-1.57 (m, 5H).

Peak 2, isomer 2: Intermediate 9-2 (58.8 mg, 29% yield). MS (ESI) m/z 624.3 [M+1]⁺. ¹H NMR (499 MHz, DMSO-d₆) δ 7.92-7.72 (m, 1H), 6.96-6.76 (m, 2H), 6.60-6.41 (m, 1H), 5.52-5.23 (m, 1H), 4.40-4.31 (m, 1H), 4.27-4.18 (m, 1H), 4.15-4.04 (m, 2H), 3.68-3.61 (m, 2H), 3.60-3.54 (m, 1H), 3.53-3.46 (m, 1H), 3.00-2.92 (m, 1H), 2.90-2.75 (m, 1H), 2.60-2.53 (m, 1H), 2.37 (d, J=1.4 Hz, 3H), 2.36-2.27 (m, 1H), 1.96-1.77 (m, 4H), 1.74-1.61 (m, 5H).

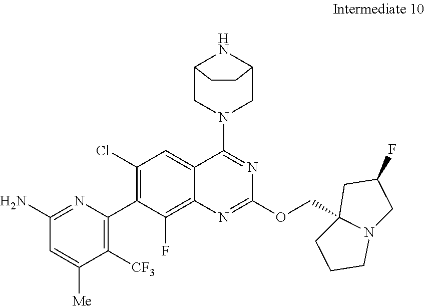 [SMILES: Cc1cc(N)nc(-c2c(Cl)cc3c(N4CC5CCC(C4)N5)nc(OC[C@@]45CCCN4C[C@H](F)C5)nc3c2F)c1C(F)(F)F]

### 6-(2-{[(2R,7aS)-2-fluoro-hexahydro-1H-pyrrolizin-7a-yl]methoxy}-6-chloro-4-{3,8-diazabicyclo[3.2.1]octan-3-yl}-8-fluoroquinazolin-7-yl)-4-methyl-5-(trifluoromethyl)pyridin-2-amine

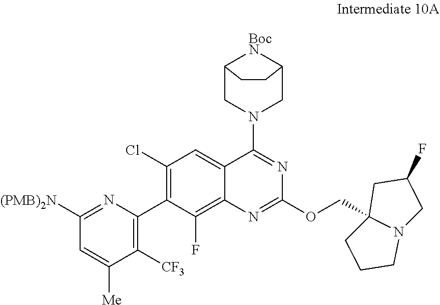

### tert-butyl 3-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

To a stirred solution of ((2R,7aS)-2-fluorotetrahydro-1H-pyrrolizin-7a(5H)-yl)methanol (commercially available, or prepared according to the procedure described in WO 2022/031678, 251 mg, 1.575 mmol) in THE (5 mL) at 0° C. was added NaH (28.4 mg, 1.181 mmol) and the mixture was stirred for an additional 30 minutes. Then, a solution of tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (650 mg, 0.788 mmol) in THE (2 mL) was added and gradually warmed to room temperature over a period of 2 h. The reaction mixture was quenched with saturated aq. ammonium chloride solution and extracted with ethyl acetate. The combined organic layers were washed with water, then brine, and then dried over Na₂SO₄ and concentrated under reduced pressure to provide a crude residue, which was purified by silica gel column chromatography using CombiFlash instrument (40 g RediSep® column, 50 to 60% EtOAc-pet ether) to provide tert-butyl 3-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (475 mg, 0.492 mmol, 62.5% yield) as a brown solid. MS (ESI) m/z: 964.2 (M+H)⁺.

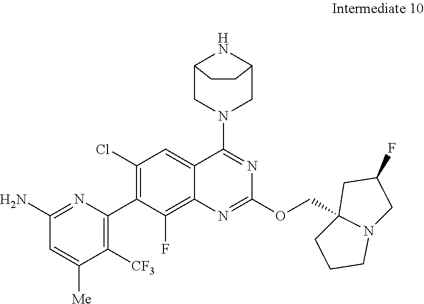 [SMILES: Cc1cc(N)nc(-c2c(Cl)cc3c(N4CC5CCC(C4)N5)nc(OC[C@@]45CCCN4C[C@H](F)C5)nc3c2F)c1C(F)(F)F]

### 6-(2-{[(2R,7aS)-2-fluoro-hexahydro-1H-pyrrolizin-7a-yl]methoxy}-6-chloro-4-{3,8-diazabicyclo[3.2.1]octan-3-yl}-8-fluoroquinazolin-7-yl)-4-methyl-5-(trifluoromethyl)pyridin-2-amine

A stirred solution of TFA (190 μL, 2.462 mmol) and triethylsilane (79 μL, 0.492 mmol) was added to tert-butyl 3-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Intermediate 10A, 475 mg, 0.492 mmol) at room temperature and the resulting reaction mixture was heated to 35° C. for 24 h. Then, the reaction mixture was concentrated under reduced pressure, co-distilled with toluene (twice), neutralised with DIPEA and concentrated under reduced pressure to provide the crude residue, which was purified by achiral SFC followed by chiral SFC to provide 6-(2-{[(2R,7aS)-2-fluoro-hexahydro-1H-pyrrolizin-7a-yl]methoxy}-6-chloro-4-{3,8-diazabicyclo[3.2.1]octan-3-yl}-8-fluoroquinazolin-7-yl)-4-methyl-5-(trifluoromethyl)pyridin-2-amine (isomer 1) (9 mg, 0.014 mmol, 2.93% yield) and (isomer 2) (8 mg, 0.013 mmol, 2.60% yield).

(Isomer 1): MS(ESI) m/z: 624.2, [M+H]⁺. ¹H NMR (400 MHz, DMSO-d₆) δ=7.82 (s, 1H), 6.84 (s, 2H), 6.49 (s, 1H), 5.27 (d, J=45.2 Hz, 1H), 4.35 (d, J=9.4 Hz, 1H), 4.27 (d, J=9.4 Hz, 1H), 4.08 (ABq, J=8.4 Hz, 2H), 3.76-3.68 (m, 2H), 3.59 (d, J=10.1 Hz, 1H), 3.51 (d, J=10.1 Hz, 1H), 3.10-3.00 (m, 3H), 2.87-2.80 (m, 1H), 2.35 (s, 3H), 2.14-2.10 (m, 1H), 2.05-2.03 (m, 1H), 2.02-1.95 (m, 1H), 1.80-1.70 (m, 7H).

Preparative achiral SFC Conditions: column/dimensions: Princeton SFC Diol (250×4.6) mm, 5u; CO₂%: 30%; Co-solvent %: 70% of (0.2% NH₃ in methanol); Flow rate: 3 mL/min; Back Pressure: 100 bar; Retention time=6.129 min.

Preparative Chiral SFC Conditions: Column/dimensions: Chiralcel ODH (250×4.6) mm, 5u; CO₂%: 30%; Co-solvent %: 70% of (5 mM NH₄OAc in ACN:methanol (1:1)); Flow rate: 4 mL/min; Back Pressure: 100 bar; Retention time of Peak-01=3.234 min & Retention time of Peak-02=6.862 min.

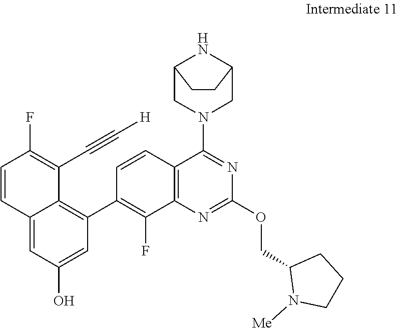 [SMILES: C#Cc1c(F)ccc2cc(O)cc(-c3ccc4c(N5CC6CCC(C5)N6)nc(OC[C@@H]5CCCN5C)nc4c3F)c12]

### 4-(4-((1R,5S)-3,8-diazabicyclo[3.2.1]octan-3-yl)-8-fluoro-2-(((S)-1-methylpyrrolidin-2-yl)methoxy)quinazolin-7-yl)-5-ethynyl-6-fluoronaphthalen-2-ol

The desired product was prepared in an analogous way to Intermediate 2, substituting tert-butyl (1R,5S)-3-(7-bromo-8-fluoro-2-(((S)-1-methylpyrrolidin-2-yl)methoxy)quinazolin-4-yl)-3,8-diazabicyclo[3.2.1]octane-8-carboxylate for tert-butyl (1R,5S)-3-(7-bromo-6-chloro-8-fluoro-2-(((S)-1-methylpyrrolidin-2-yl)methoxy)quinazolin-4-yl)-3,8-diazabicyclo[3.2.1]octane-8-carboxylate (prepared as described in WO2021106231) as starting material. LCMS (ESI) m/z: [M+H]⁺ calcd for C₃₂H₃₂F₂N₅O₂ 556.2. found 556.1; ¹H NMR (500 MHz, CD₃OD) δ 7.87-7.83 (m, 2H), 7.41-7.36 (m, 1H), 7.34-7.29 (m, 2H), 7.10 (t, J=3.0 Hz, 1H), 4.71-4.62 (m, 4H), 4.17-4.12 (m, 3H), 3.89-3.79 (m, 4H), 3.21-3.17 (m, 1H), 3.04 (s, 3H), 2.43-2.34 (m, 2H), 2.20-2.00 (m, 6H).

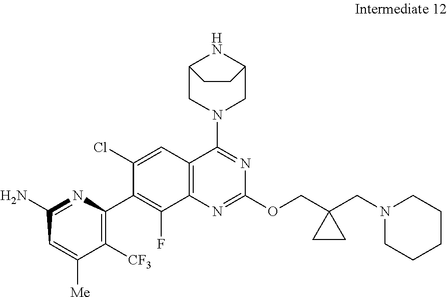

### (5M)-6-(6-chloro-4-{3,8-diazabicyclo[3.2.1]octan-3-yl}-8-fluoro-2-({1-[(piperidin-1-yl)methyl]cyclopropyl}methoxy)quinazolin-7-yl)-4-methyl-5-(trifluoromethyl)pyridin-2-amine

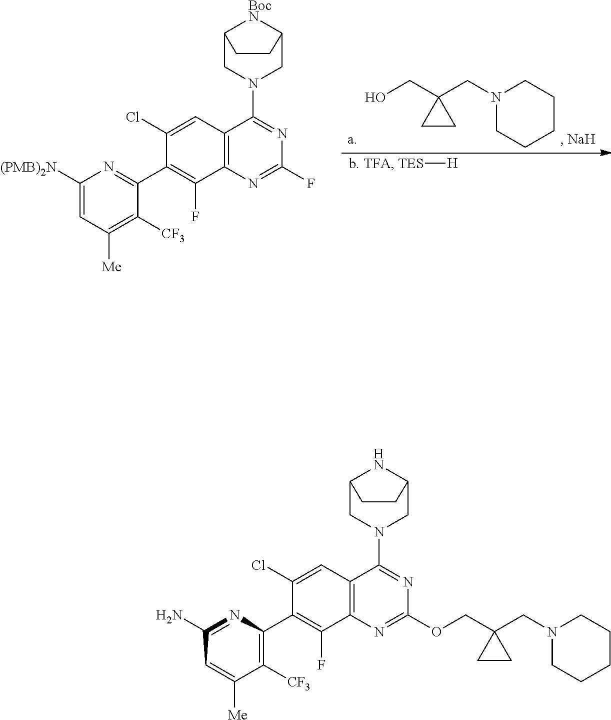

### Step 1: tert-butyl (1R,5S)-3-(7-(6-(bis(4-methoxybenzyl)amino)-4-methyl-3-(trifluoromethyl)pyridin-2-yl)-6-chloro-8-fluoro-2-((1-(piperidin-1-ylmethyl)cyclopropyl)methoxy)quinazolin-4-yl)-3,8-diazabicyclo[3.2.1]octane-8-carboxylate

(1-(Piperidin-1-ylmethyl)cyclopropyl)methanol (51 mg, 0.30 mmol) was dissolved in THF (10 mL) and sodium hydride (60% dispersion in mineral oil, 13 mg, 0.33 mmol) was added. The reaction mixture was stirred at room temperature for 1 h. The solution was cooled to 0° C. and tert-butyl (1R,5S)-3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (250 mg, 0.30 mmol) was added dropwise as a solution in THE (2 mL). The reaction mixture was stirred at 0° C. for 1 h. The reaction mixture was quenched with water (20 mL) and ethyl acetate (100 mL) was added. The layers were separated and the organic phase was washed with brine, dried over sodium sulfate, filtered, and concentrated to provide the desired product, which was used directly in the next step without additional purification (quantitative yield assumed). LC/MS (ESI) m/z: [M+H]⁺ calcd for C₅₂H₆ClF₄N₇O₅ 974.4. found 974.4.

### Step 2: (5M)-6-(6-chloro-4-{3,8-diazabicyclo[3.2.1]octan-3-yl}-8-fluoro-2-({1-[(piperidin-1-yl)methyl]cyclopropyl}methoxy)quinazolin-7-yl)-4-methyl-5-(trifluoromethyl)pyridin-2-amine

Triethylsilane (1 mL, 6.3 mmol) and TFA (2 mL, 26.0 mmol) were combined as liquids and stirred for 30 min. To this solution was added tert-butyl (1R,5S)-3-(7-(6-(bis(4-methoxybenzyl)amino)-4-methyl-3-(trifluoromethyl)pyridin-2-yl)-6-chloro-8-fluoro-2-((1-(piperidin-1-ylmethyl)cyclopropyl)methoxy)quinazolin-4-yl)-3,8-diazabicyclo[3.2.1]octane-8-carboxylate (from Step 1, 180 mg, 0.19 mmol). The reaction mixture was heated at 40° C. for 24 h. The reaction mixture was directly concentrated and was purified by preparative HPLC to provide the desired product (50 mg, 0.08 mmol, 43% yield) as a white solid. ¹H NMR (400 MHz, DMSO-d₆) δ 7.80 (d, J=1.2 Hz, 1H), 6.83 (s, 2H), 6.49 (s, 1H), 4.30 (d, J=12.0 Hz, 1H), 4.27-4.23 (m, 2H), 4.19 (d, J=12.0 Hz, 1H), 3.55-3.40 (m, 4H), 2.37-2.25 (m, 9H), 1.70-1.55 (m, 4H), 1.47-1.39 (m, 4H), 1.38-1.29 (m, 2H), 0.63-0.60 (m, 2H), 0.38-0.36 (m, 2H).

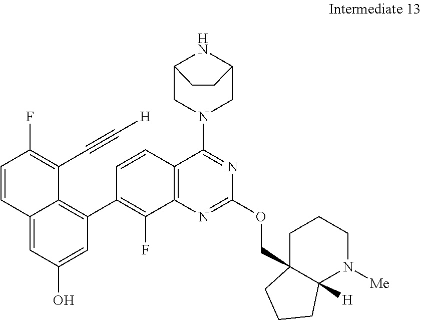 [SMILES: C#Cc1c(F)ccc2cc(O)cc(-c3ccc4c(N5CC6CCC(C5)N6)nc(OC[C@]56CCC[C@H]5N(C)CCC6)nc4c3F)c12]

### 4-(4-((1R,5S)-3,8-diazabicyclo[3.2.1]octan-3-yl)-8-fluoro-2-(((4aS,7aR)-1-methyloctahydro-4aH-cyclopenta[b]pyridin-4a-yl)methoxy)quinazolin-7-yl)-5-ethynyl-6-fluoronaphthalen-2-ol

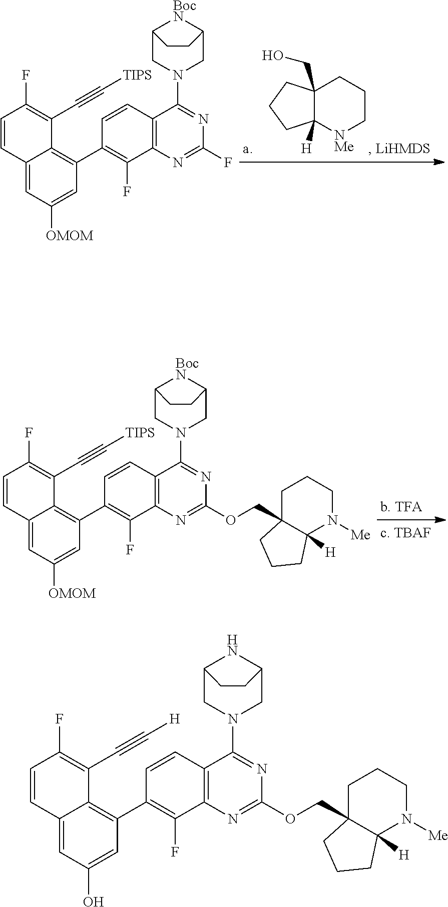

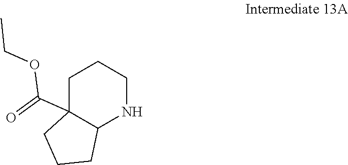

### Ethyl octahydro-4aH-cyclopenta[b]pyridine-4a-carboxylate

Intermediate 13A was prepared according to the procedure in Molecules 2017, 22:827, Compound 25a.

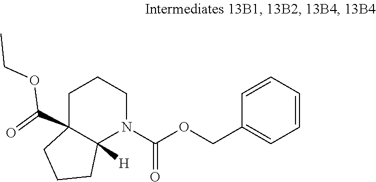 [SMILES: CCOC(=O)[C@]12CCC[C@H]1N(C(=O)OCc1ccccc1)CCC2]

### 1-benzyl 4a-ethyl (4aS,7aR)-hexahydro-1H-cyclopenta[b]pyridine-1,4a(2H)-dicarboxylate (peak 2)

To a solution of ethyl octahydro-4aH-cyclopenta[b]pyridine-4a-carboxylate (Intermediate 13A, 1.7 g, 8.62 mmol) and TEA (2.402 mL, 17.23 mmol) in THE (10 mL) was added N-(benzyloxycarbonyl)succinimide (1.718 g, 6.89 mmol) and the mixture was stirred at room temperature for 18 hours. The mixture was diluted with EtOAc (15 mL) and was washed with a solution of aqueous saturated sodium bicarbonate (2×15 mL). The ethyl acetate layer was dried over sodium sulfate, filtered and concentrated. The crude product was subjected to ISCO flash chromatography (silica gel/DCM-20% MeOH/DCM 100:0 to 50:50 gradient) to yield 1-benzyl 4a-ethyl hexahydro-1H-cyclopenta[b]pyridine-1,4a(2H)-dicarboxylate (2.20 g, 6.64 mmol, 77% yield). 1-Benzyl 4a-ethyl hexahydro-1H-cyclopenta[b]pyridine-1,4a(2H)-dicarboxylate (2.20 g) was subjected to SFC chiral separation. [column: Cellulose-4 (5×25 cm, 5 μm) method=CO₂/IPA:heptane (1:3) with 0.1% ammonia hydroxide. 320 mL/min.] to provide:

Peak 1 (19B1): 1-benzyl 4a-ethyl hexahydro-1H-cyclopenta[b]pyridine-1,4a(2H)-dicarboxylate (625 mg, 1.792 mmol, 20.79% yield). LCMS (ESI) m/z: 332.3 [M+H]⁺ LC retention time: 1.05 min (Waters Acquity UPLC BEH C18, 2.1×50 mm, 1.7 μm particles; Mobile Phase A: water with 0.05% TFA; Mobile Phase B: ACN with 0.05% TFA; Gradient: 2-98% B over 1 minute, then a 0.5 minute hold at 98% B; Flow: 0.8 mL/min; Detection: MS and UV (220 nm)). ¹H NMR (499 MHz, CHLOROFORM-d) δ 7.41-7.28 (m, 5H), 5.17 (br s, 2H), 4.13 (br d, J=6.4 Hz, 2H), 2.86 (br s, 1H), 2.15 (br d, J=10.8 Hz, 1H), 2.03-1.90 (m, 1H), 1.90-1.74 (m, 4H), 1.73-1.63 (m, 1H), 1.58-1.42 (m, 3H), 1.27-1.14 (m, 4H), 0.98-0.68 (m, 1H).

Peak 2 (19B2): 1-benzyl 4a-ethyl (4aS,7aR)-hexahydro-1H-cyclopenta[b]pyridine-1,4a(2H)-dicarboxylate (640 mg, 1.835 mmol, 21.29% yield). LCMS (ESI) m/z: 332.3 [M+H]⁺ LC retention time: 1.05 min (Waters Acquity UPLC BEH C18, 2.1×50 mm, 1.7 μm particles; Mobile Phase A: water with 0.05% TFA; Mobile Phase B: ACN with 0.05% TFA; Gradient: 2-98% B over 1 minute, then a 0.5 minute hold at 98% B; Flow: 0.8 mL/min; Detection: MS and UV (220 nm)). ¹H NMR (499 MHz, CHLOROFORM-d) δ 7.41-7.28 (m, 5H), 5.17 (br s, 2H), 4.13 (br d, J=6.4 Hz, 2H), 2.86 (br s, 1H), 2.15 (br d, J=10.8 Hz, 1H), 2.03-1.90 (m, 1H), 1.90-1.74 (m, 4H), 1.73-1.63 (m, 1H), 1.58-1.42 (m, 3H), 1.27-1.14 (m, 4H), 0.98-0.68 (m, 1H).

Peak 3 (19B3): 1-benzyl 4a-ethyl hexahydro-1H-cyclopenta[b]pyridine-1,4a(2H)-dicarboxylate (130 mg, 0.373 mmol, 4.32% yield). LCMS (ESI) m/z: 332.3 [M+H]*LC retention time: 1.05 min (Waters Acquity UPLC BEH C18, 2.1×50 mm, 1.7 μm particles; Mobile Phase A: water with 0.05% TFA; Mobile Phase B: ACN with 0.05% TFA; Gradient: 2-98% B over 1 minute, then a 0.5 minute hold at 98% B; Flow: 0.8 mL/min; Detection: MS and UV (220 nm)). ¹H NMR (499 MHz, CHLOROFORM-d) δ 7.40-7.28 (m, 5H), 5.18-5.10 (m, 2H), 4.38 (ddd, J=13.4, 2.5, 1.4 Hz, 1H), 4.13-4.05 (m, 2H), 2.88 (dd, J=12.8, 7.0 Hz, 1H), 2.79-2.67 (m, 2H), 2.42 (dt, J=13.1, 2.7 Hz, 1H), 2.28-2.14 (m, 2H), 1.76-1.55 (m, 4H), 1.46-1.26 (m, 2H), 1.20 (t, J=7.2 Hz, 3H).

Peak 4 (19B4): 1-benzyl 4a-ethyl hexahydro-1H-cyclopenta[b]pyridine-1,4a(2H)-dicarboxylate (141 mg, 0.404 mmol, 4.69% yield). LCMS (ESI) m/z: 332.3 [M+H]*LC retention time: 1.05 min (Waters Acquity UPLC BEH C18, 2.1×50 mm, 1.7 μm particles; Mobile Phase A: water with 0.05% TFA; Mobile Phase B: ACN with 0.05% TFA; Gradient: 2-98% B over 1 minute, then a 0.5 minute hold at 98% B; Flow: 0.8 mL/min; Detection: MS and UV (220 nm)). ¹H NMR (499 MHz, CHLOROFORM-d) δ 7.40-7.28 (m, 5H), 5.18-5.10 (m, 2H), 4.38 (ddd, J=13.4, 2.5, 1.4 Hz, 1H), 4.13-4.05 (m, 2H), 2.88 (dd, J=12.8, 7.0 Hz, 1H), 2.79-2.67 (m, 2H), 2.42 (dt, J=13.1, 2.7 Hz, 1H), 2.28-2.14 (m, 2H), 1.76-1.55 (m, 4H), 1.46-1.26 (m, 2H), 1.20 (t, J=7.2 Hz, 3H).

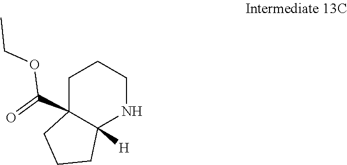

### Ethyl (4aS,7aR)-octahydro-4aH-cyclopenta[b]pyridine-4a-carboxylate

A mixture of 1-benzyl 4a-ethyl (4aS,7aR)-hexahydro-1H-cyclopenta[b]pyridine-1,4a(2H)-dicarboxylate (Intermediate 13B2, 640 mg, 1.931 mmol) and 10% Pd—C(103 mg, 0.097 mmol) in MeOH (10 mL) was hydrogenated under 1 atm of hydrogen for 18 hours. The Pd/C was filtered off and the filtrate was concentrated to provide crude ethyl (4aS,7aR)-octahydro-4aH-cyclopenta[b]pyridine-4a-carboxylate (385 mg, 1.854 mmol, 96% yield) as a clear oil. ¹H NMR (499 MHz, CHLOROFORM-d) δ 4.17 (dtt, J=10.6, 7.1, 3.6 Hz, 2H), 3.57 (t, J=6.1 Hz, 1H), 2.90 (ddd, J=13.0, 7.7, 3.7 Hz, 1H), 2.71 (ddd, J=13.0, 7.0, 3.6 Hz, 1H), 2.01-1.92 (m, 2H), 1.84-1.62 (m, 7H), 1.60-1.40 (m, 2H), 1.28 (t, J=7.1 Hz, 3H).

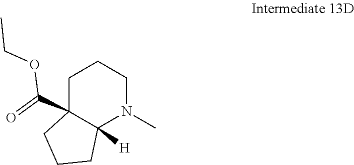

### Ethyl (4aS,7aR)-1-methyloctahydro-4aH-cyclopenta[b]pyridine-4a-carboxylate

To a solution of ethyl (4aS,7aR)-octahydro-4aH-cyclopenta[b]pyridine-4a-carboxylate (Intermediate 13C, 385 mg, 1.952 mmol) and formaldehyde solution, 37 wt. % in H₂O (176 mg, 5.85 mmol) in MeOH (5.0 mL) was added sodium cyanoborohydride (123 mg, 1.952 mmol) and the mixture was stirred at room temperature for 18 hours. The mixture was then concentrated. The mixture was diluted with EtOAc (5 mL) and was washed with a solution of aqueous saturated sodium carbonate (2×5 mL). The ethyl acetate layer was dried over sodium sulfate, filtered and concentrated to provide crude ethyl (4aS,7aR)-1-methyloctahydro-4aH-cyclopenta[b]pyridine-4a-carboxylate (380 mg, 1.798 mmol, 92% yield). ¹H NMR (499 MHz, CHLOROFORM-d) δ 4.24-4.12 (m, 2H), 3.29 (t, J=6.4 Hz, 1H), 2.60-2.51 (m, 1H), 2.36-2.28 (m, 3H), 2.00-1.88 (m, 2H), 1.83-1.60 (m, 8H), 1.56-1.40 (m, 1H), 1.32-1.26 (m, 3H).

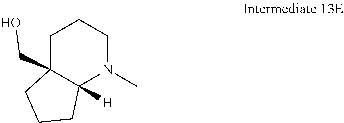

### ((4aS,7aR)-1-methyloctahydro-4aH-cyclopenta[b]pyridin-4a-yl)methanol

To a solution of ethyl (4aS,7aR)-1-methyloctahydro-4aH-cyclopenta[b]pyridine-4a-carboxylate (Intermediate 13D, 380 mg, 1.798 mmol) in anhydrous THE (2.0 mL) was added a solution of lithium aluminum hydride solution 1.0 M in THE (4496 μl, 4.50 mmol) and the mixture was stirred at room temperature for 18 hours. Brine (0.3 mL) was added dropwise to the mixture. EtOAc (5.0 mL) was added to the mixture. The precipitate was filtered off and the filtrate was concentrated to provide crude ((4aS,7aR)-1-methyloctahydro-4aH-cyclopenta[b]pyridin-4a-yl)methanol (327 mg, 1.739 mmol, 97% yield). ¹H NMR (499 MHz, CHLOROFORM-d) δ 3.69-3.62 (m, 2H), 2.87 (t, J=7.6 Hz, 1H), 2.51 (td, J=11.1, 3.4 Hz, 1H), 2.43-2.35 (m, 1H), 2.30 (s, 3H), 2.01-1.85 (m, 2H), 1.82-1.74 (m, 1H), 1.68-1.52 (m, 6H), 1.47-1.42 (m, 1H), 1.39-1.33 (m, 1H).

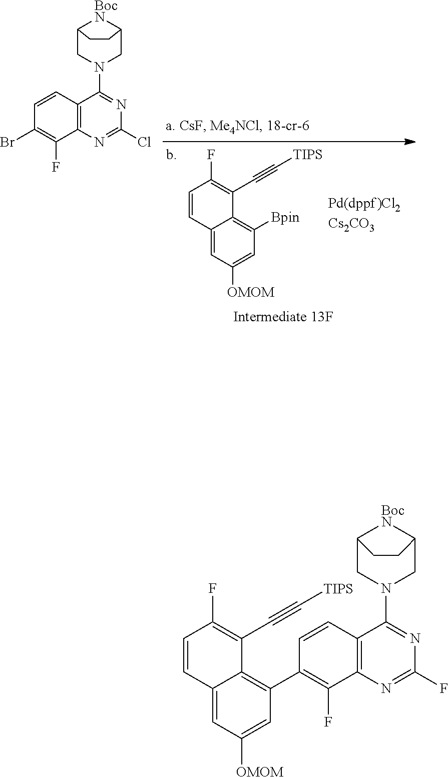

### tert-butyl (1R,5S)-3-(2,8-difluoro-7-(7-fluoro-3-(methoxymethoxy)-8-((triisopropylsilyl)ethynyl)naphthalen-1-yl)quinazolin-4-yl)-3,8-diazabicyclo[3.2.1]octane-8-carboxylate

### Step 1: tert-butyl (1R,5S)-3-(7-bromo-2,8-difluoroquinazolin-4-yl)-3,8-diazabicyclo[3.2.1]octane-8-carboxylate

tert-Butyl (1R,5S)-3-(7-bromo-2-chloro-8-fluoroquinazolin-4-yl)-3,8-diazabicyclo[3.2.1]octane-8-carboxylate (42 g, 89 mmol, prepared according to procedures described in WO2021/136231 A1) was dissolved in MeCN (420 mL) and DMA (63 mL). Cesium fluoride (67.6 g, 445 mmol), tetramethylammonium chloride (1.0 g, 8.9 mmol), and 18-crown-6 (2.4 g, 8.9 mmol) were added as solids. The reaction mixture was heated at 60° C. for 16 h. The reaction mixture was diluted with EtOAc (1 μL) and washed with water (2×500 mL) and brine (500 mL). The organic phase was dried over sodium sulfate, filtered, and concentrated to provide the desired product (26.5 g, 58 mmol, 65% yield), which was of sufficient purity to be used directly in the next step, without additional purification LC/MS (ESI) m/z: [M+H]⁻ calcd for C₁₉H₂₂BrF₂N₄O₂ 455.1. found 455.2.

### Step 2: tert-Butyl (1R,5S)-3-(2,8-difluoro-7-(7-fluoro-3-(methoxymethoxy)-8-((triisopropylsilyl)ethynyl)naphthalen-1-yl)quinazolin-4-yl)-3,8-diazabicyclo[3.2.]octane-8-carboxylate

tert-Butyl (1R,5S)-3-(7-bromo-2,8-difluoroquinazolin-4-yl)-3,8-diazabicyclo[3.2.1]octane-8-carboxylate (from Step 1, 5.0 g, 11.0 mmol) was dissolved in dioxane (135 mL) and water (15 mL). ((2-fluoro-6-(methoxymethoxy)-8-(4,4,5,5-tetramethyl-1,3,2-dioxaborolan-2-yl)naphthalen-1-yl)ethynyl)triisopropylsilane (8.4 g, 16.5 mmol) and cesium carbonate (11 g, 33 mmol) were added and the resulting solution was sparged with nitrogen for 15 min. Pd(dppf)Cl₂ (0.8 g, 1.1 mmol) was added and the reaction mixture was heated at 100° C. for 16 h. Water (50 mL) and EtOAc (100 mL) were added and the layers were separated. The aqueous layer was further extracted with EtOAc (2×100 mL) and the combined organic phases were dried over sodium sulfate, filtered, and concentrated. The crude residue was purified by reverse phase HPLC to provide the desired product (3.0 g, 3.9 mmol, 35% yield); LC/MS (ESI) m/z: [M+H]⁺ calcd for C₄₂H₅₂F₃N₄O₄Si 761.4. found 761.2.

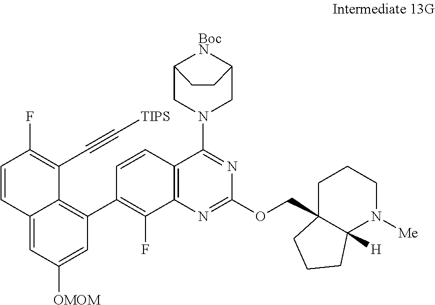

### tert-butyl (1R,5S)-3-(8-fluoro-7-(7-fluoro-3-(methoxymethoxy)-8-((triisopropylsilyl)ethynyl)naphthalen-1-yl)-2-(((4aS,7aR)-1-methyloctahydro-4aH-cyclopenta[b]pyridin-4a-yl)methoxy)quinazolin-4-yl)-3,8-diazabicyclo[3.2.1]octane-8-carboxylate

tert-Butyl (1R,5S)-3-(2,8-difluoro-7-(7-fluoro-3-(methoxymethoxy)-8-((triisopropylsilyl)ethynyl)naphthalen-1-yl)quinazolin-4-yl)-3,8-diazabicyclo[3.2.1]octane-8-carboxylate (Intermediate 13F, 45 mg, 0.06 mmol) and ((4aS,7aR)-1-methyloctahydro-4aH-cyclopenta[b]pyridin-4a-yl)methanol (Intermediate 13E, 10 mg, 0.06 mmol) were combined and dissolved in THE (1.0 mL). Lithium bis(trimethylsilylamide) solution (1.0 M in THF, 90 μL, 0.09 mmol) was added and the reaction mixture was stirred at room temperature for 18 h. The solution was directly concentrated and was purified by preparative HPLC to provide the desired product (35 mg, 0.04 mmol, 65% yield); LC/MS (ESI) m/z: [M+H]⁺ calcd for C₅₂H₇₀F₂N₅O₅Si 910.5. found 910.6.

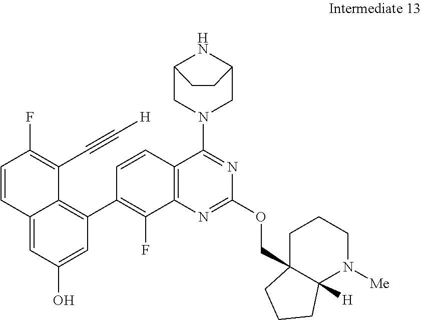 [SMILES: C#Cc1c(F)ccc2cc(O)cc(-c3ccc4c(N5CC6CCC(C5)N6)nc(OC[C@]56CCC[C@H]5N(C)CCC6)nc4c3F)c12]

### 4-(4-((1R,5S)-3,8-diazabicyclo[3.2.1]octan-3-yl)-8-fluoro-2-(((4aS,7aR)-1-methyloctahydro-4aH-cyclopenta[b]pyridin-4a-yl)methoxy)quinazolin-7-yl)-5-ethynyl-6-fluoronaphthalen-2-ol

tert-Butyl (1R,5S)-3-(8-fluoro-7-(7-fluoro-3-(methoxymethoxy)-8-((triisopropylsilyl)ethynyl)naphthalen-1-yl)-2-(((4aS,7aR)-1-methyloctahydro-4aH-cyclopenta[b]pyridin-4a-yl)methoxy)quinazolin-4-yl)-3,8-diazabicyclo[3.2.1]octane-8-carboxylate (Intermediate 13F, 35 mg, 0.04 mmol) was dissolved in DCM (1.0 mL) and TFA (1.0 mL). The reaction mixture was stirred at room temperature for 30 min. The reaction mixture was directly concentrated and resuspended in THE (100 μL). TBAF solution (1.0 M in THF, 770 μL, 0.77 mmol) was added and the reaction mixture was stirred at room temperature for 1 h. The reaction mixture was directly concentrated and was purified by preparative HPLC to provide the desired product (9.6 mg, 0.015 mmol, 39% yield); LC/MS (ESI) m/z: [M+H]⁺ calcd for C₃₆H₃₈F₂—N₅O₂ 610.3. found 610.5; ¹H NMR (500 MHz, CD₃OD) δ 7.85-7.81 (m, 1H), 7.79-7.74 (m, 1H), 7.32-7.27 (m, 2H), 7.26-7.20 (m, 1H), 7.09 (d, J=2.5 Hz, 1H), 4.58-4.42 (m, 3H), 4.38-4.27 (m, 1H), 3.67-3.56 (m, 4H), 3.13-2.97 (m, 1H), 2.85-2.71 (m, 1H), 2.63-2.51 (m, 1H), 2.49-2.37 (m, 3H), 2.03-1.94 (m, 1H), 1.94-1.90 (m, 1H), 1.90-1.81 (m, 6H), 1.81-1.68 (m, 6H), 1.63-1.55 (m, 1H).

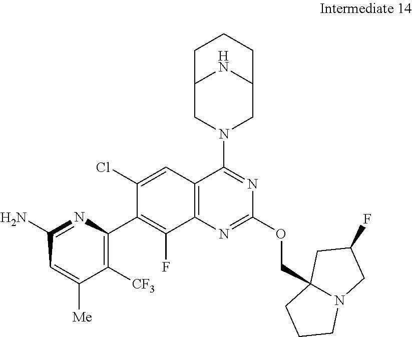

### 6-(4-(3,9-diazabicyclo[3.3.1]nonan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine

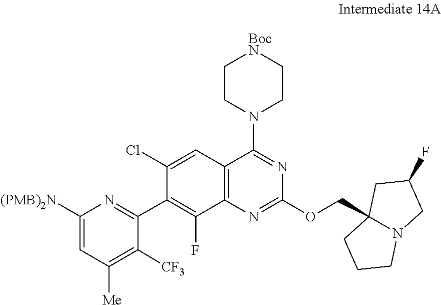

### tert-butyl 4-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)piperazine-1-carboxylate

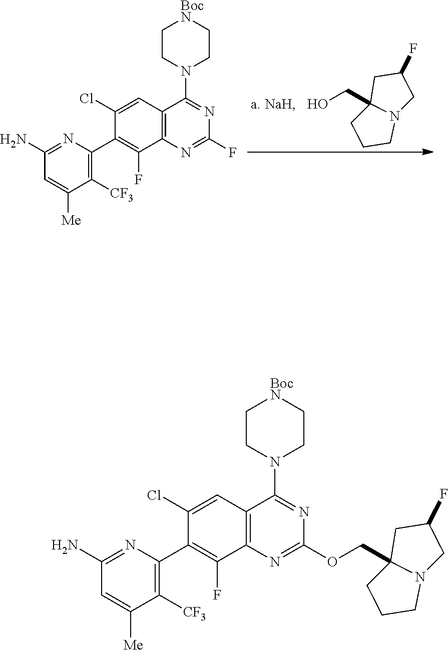

((2R,7aR)-2-Fluorotetrahydro-1H-pyrrolizin-7a(5H)-yl)methanol (Intermediate 9G, 110 mg, 0.68 mmol) was dissolved in THF (5 mL) and the solution was cooled to 0° C. Sodium hydride (60 wt % dispersion in mineral oil, 27 mg, 0.68 mmol) was added portionwise as a solid and the reaction mixture was stirred for 30 min. Tert-butyl 4-(7-(6-(bis(4-methoxybenzyl)amino)-4-methyl-3-(trifluoromethyl)pyridin-2-yl)-6-chloro-2,8-difluoroquinazolin-4-yl)piperazine-1-carboxylate (Intermediate 6G, 300 mg, 0.375 mmol) was added and the reaction mixture was allowed to warm to room temperature. After stirring for 2 h at room temperature, the reaction mixture was quenched with water (20 mL) and diluted with EtOAc (25 mL). The layers were separated and the aqueous phase was further extracted with EtOAc (25 mL). The combined organic phases were washed with brine (20 mL), dried over sodium sulfate, filtered, and concentrated. The crude residue was purified by column chromatogrpahy on neutral alumina (70% EtOAc/hexanes) to provide the desired product (200 mg, 44% yield). LC/MS (ESI) m/z: [M+H]⁺ calcd for C₄₈H₅₄ClF₅N₇O₅ 938.4. found 938.2.

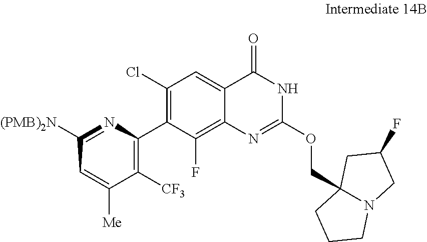

### 7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one

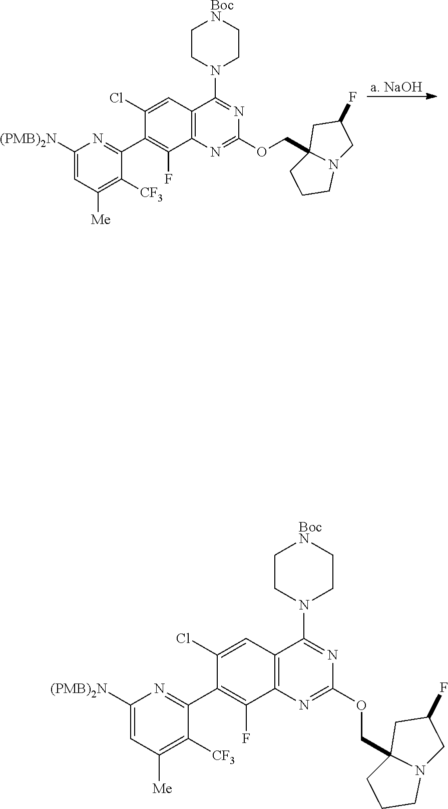

tert-Butyl 4-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)piperazine-1-carboxylate (Intermediate 14A, 8.8 g, 9.38 mmol) was dissolved in EtOH (30 mL) and sodium hydroxide (11.2 g, 28 mmol) was added as a solid. The reaction mixture was heated at 80° C. for 36 h. The reaction mixture was directly concentrated and the crude material was taken up in water (80 mL) and was extracted with EtOAc (3×150 mL). The combined organic phases were washed with brine (50 mL), dried over sodium sulfate, filtered, and concentrated. The crude residue was purified by reverse phase column chromatography (55% MeCN in water) to provide the product as a mixture of atrop isomers. The material was further purified by chiral SFC to provide the desired isomer (retention time=4.72 min, 1.2 g, 1.56 mmol, 17% yield) and a second isomer (retention time=5.85 min, 1.3 g, 1.69 mmol, 18% yield). LC/MS (ESI) m/z: [M+H]⁺ calcd for C₃₉H₃₈F₂₅N₅O₄ 770.2. found 770.2.

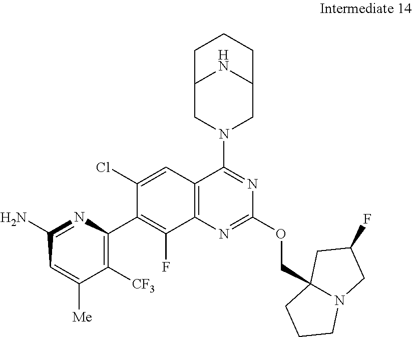

### 6-(4-(3,9-diazabicyclo[3.3.1]nonan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine

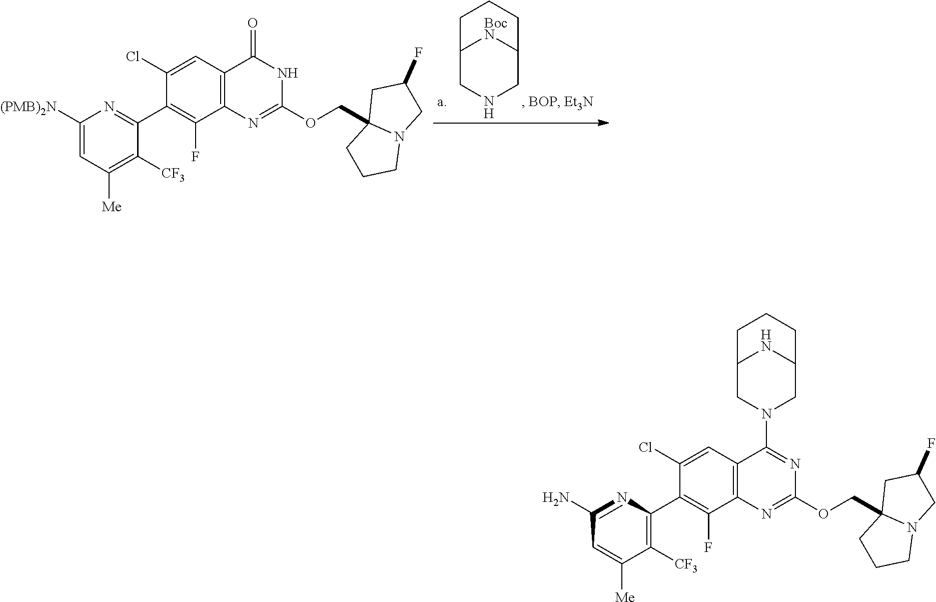

### Step 1: tert-butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,9-diazabicyclo[3.3.1]nonane-9-carboxylate

7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 60 mg, 0.08 mmol) and benzotriazol-1-yloxy)tris(dimethylamino)phosphonium hexafluorophosphate (52 mg, 0.12 mmol) were combined and dissolved in acetonitrile (4 mL). Triethylamine (10 μL, 0.08 mmol) was added followed by tert-butyl 3,9-diazabicyclo[3.3.1]nonane-9-carboxylate (35 mg, 0.16 mmol). The reaction mixture was heated at 35° C. for 10 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was purified by column chromatography (60→100% EtOAc/hexanes) to provide the desired product (65 mg, 0.06 mmol, 73% yield); LC/MS (ESI) m/z: [M+H]⁺ calcd for C₅₁H₅₈ClF₅N₇O₅ 978.4. found 977.8.

### Step 2: 6-(4-(3,9-diazabicyclo[3.3.1]nonan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine

Tert-Butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,9-diazabicyclo[3.3.1]nonane-9-carboxylate (from Step 1, 60 mg, 0.06 mmol) was dissolved in TFA (1.5 mL), and triethylsilane (1 μL, 0.006 mmol) and water (0.05 mL) were added. The reaction mixture was heated at 40° C. for 16 h. The crude mixture was directly purified by preparative HPLC to provide the desired product (17 mg, 0.02 mmol, 39% yield). LC/MS (ESI) m/z: [M+H]⁺ calcd for C₃₀H₃₄ClF₅N₇O 638.2. found 638.8; ¹H NMR (400 MHz, CD₃OD) δ 8.05-7.81 (m, 1H), 6.63 (s, 1H), 5.49-5.25 (m, 1H), 4.72 (br dd, J=8.6, 13.6 Hz, 2H), 4.57-4.41 (m, 2H), 3.85 (ddd, J=3.3, 6.7, 14.0 Hz, 2H), 3.67-3.47 (m, 3H), 3.31-3.23 (m, 2H), 3.19-3.11 (m, 1H), 3.09-2.94 (m, 2H), 2.69-2.54 (m, 1H), 2.47 (d, J=1.4 Hz, 2H), 2.32-2.19 (m, 1H), 2.17-2.01 (m, 6H), 1.99-1.87 (m, 3H).

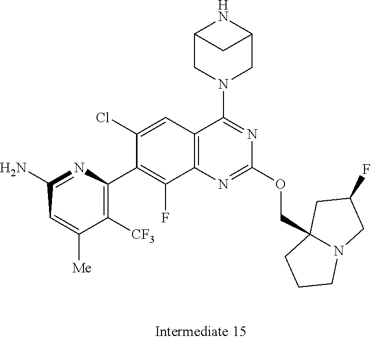

### 6-(4-(3,6-diazabicyclo[3.1.1]heptan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine

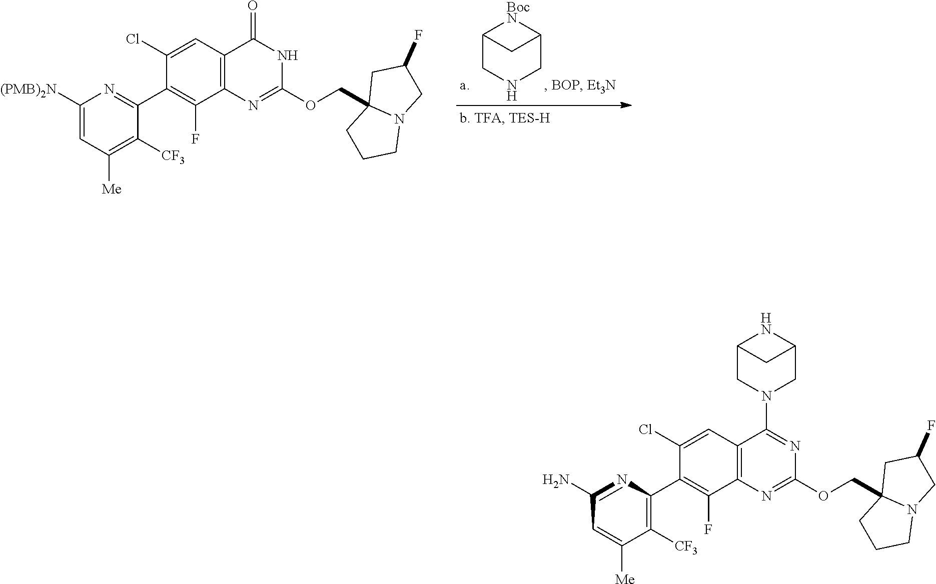

### Step 1: tert-butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.1.1]heptane-6-carboxylate

7-(6-(Bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 60 mg, 0.08 mmol) and benzotriazol-1-yloxy)tris(dimethylamino)phosphonium hexafluorophosphate (52 mg, 0.12 mmol) were combined and dissolved in acetonitrile (4 mL). Triethylamine (10 μL, 0.08 mmol) was added followed by tert-butyl 3,6-diazabicyclo[3.1.1]heptane-6-carboxylate (19 mg, 0.10 mmol). The reaction mixture was heated at 35° C. for 10 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was purified by column chromatography (40→100% EtOAc/hexanes) to provide the desired product (60 mg, 0.06 mmol, 75% yield). LC/MS (ESI) m/z: [M+H]⁺ calcd for C₄₉H₅₄ClF₅N₇O₅ 950.4. found 950.2.

### Step 2: 6-(4-(3,6-diazabicyclo[3.1.1]heptan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine

tert-Butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.1.1]heptane-6-carboxylate (from Step 1, 60 mg, 0.06 mmol) was dissolved in TFA (1.5 mL), and triethylsilane (1 μL, 0.006 mmol) and water (0.05 mL) were added. The reaction mixture was heated at 40° C. for 16 h. The crude mixture was directly purified by preparative HPLC to provide the desired product as its TFA salt (14 mg, 0.02 mmol, 31% yield); LC/MS (ESI) m/z: [M+H]⁺ calcd for C₂₈H₃₀ClF₅N₇O 610.2. found 610.2; ¹H NMR (400 MHz, CD₃OD) δ 8.38 (d, J=1.60 Hz, 1H), 6.66 (s, 1H), 5.49-5.60 (m, 1H), 4.87-4.88 (m, 4H), 4.62-4.81 (m, 5H), 3.91-4.12 (m, 1H), 3.69-3.78 (m, 1H), 3.50-3.50 (m, 1H), 3.09-3.18 (m, 1H), 2.69-2.76 (m, 1H), 2.48-2.48 (m, 5H), 2.29-2.48 (m, 3H), 2.03-2.06 (m, 1H).

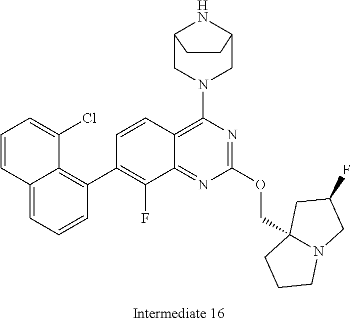 [SMILES: Fc1c(-c2cccc3cccc(Cl)c23)ccc2c(N3CC4CCC(C3)N4)nc(OC[C@@]34CCCN3C[C@H](F)C4)nc12]

### tert-butyl (1R,5S)-3-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

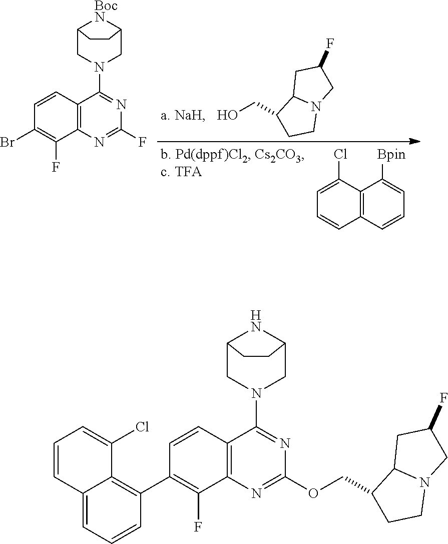

### Step 1: tert-Butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

((2R,7aS)-2-Fluorotetrahydro-1H-pyrrolizin-7a(5H)-yl)methanol (commercially available, or prepared according to the procedure described in WO 2022/031678, 2.0 g, 12.6 mmol) was dissolved in THE (60 mL) and the solution was cooled to 0° C. Sodium hydride (60 wt % dispersion in mineral oil, 0.9 g, 22.8 mmol) was added portionwise as a solid and the reaction mixture was stirred for 30 min. tert-butyl (1R,5S)-3-(7-bromo-2,8-difluoroquinazolin-4-yl)-3,8-diazabicyclo[3.2.1]octane-8-carboxylate (5.2 g, 11.4 mmol, prepared according to procedures described in WO2021/136231 A1) was added and the reaction mixture was allowed to warm to room temperature. After stirring for 24 h at room temperature, the reaction mixture was quenched with saturated ammonium chloride solution (50 mL) and diluted with EtOAc (75 mL). The layers were separated and the aqueous phase was further extracted with EtOAc (2×75 mL). The combined organic phases were washed with brine (50 mL), dried over sodium sulfate, filtered, and concentrated. The crude residue was purified by trituration with diethyl ether to provide the desired product (5.0 g, 8.2 mmol, 72% yield). LC/MS (ESI) m/z: [M+H]⁺ calcd for C₂₇H₃₅BrF₂N₅O₃ 594.2; found 594.2; ¹H NMR (400 MHz, DMSO-d₆) δ 7.73 (d, J=1.20 Hz, 1H), 7.46 (d, J=6.40 Hz, 1H), 5.21-5.35 (m, 1H), 4.23-4.29 (m, 5H), 4.10 (d, J=10.40 Hz, 1H), 4.02 (d, J=10.00 Hz, 1H), 2.00 (t, J=12.40 Hz, 2H), 3.01-3.10 (m, 2H), 2.12 (d, J=Hz, 1H), 2.13-2.00 (m, 2H), 1.75-1.85 (m, 6H), 1.67 (t, J=8.40 Hz, 2H), 1.46 (s, 9H).

### Step 2: tert-butyl (1R,5S)-3-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

tert-Butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (from Step 1, 100 mg, 0.17 mmol) was dissolved in dioxane (2 mL) and water (0.5 mL). 2-(8-Chloronaphthalen-1-yl)-4,4,5,5-tetramethyl-1,3,2-dioxaborolane (63 mg, 0.22 mmol) and cesium carbonate (164 mg, 0.51 mmol) were added and the resulting solution was sparged with nitrogen for 15 min. Pd(dppf)Cl₂ (12 mg, 0.02 mmol) was added and the reaction mixture was heated at 100° C. for 5 h. Water (5 mL) and EtOAc (5 mL) were added and the layers were separated. The aqueous layer was further extracted with EtOAc (2×5 mL) and the combined organic phases were dried over sodium sulfate, filtered, and concentrated. The crude residue was purified by column chromatography (40% EtOAc/hexanes) to provide the desired product (70 mg, 0.09 mmol, 56% yield); LC/MS (ESI) m/z: [M+H]⁺ calcd for C₃₇H₄₁ClF₂N₅O₃ 676.3. found 676.3.

### Step 3: 4-((1R,5S)-3,8-diazabicyclo[3.2.1]octan-3-yl)-7-(8-chloronaphthalen-1-yl)-8-fluoro-2-(((2R,7aS)-2-fluorotetrahydro-1H-pyrrolizin-7a(5H)-yl)methoxy)quinazoline

tert-Butyl (1R,5S)-3-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (from Step 2, 70 mg, 0.1 mmol) was dissolved in DCM (2 mL) and HCl solution (4.0 M in dioxane, 0.13 mL, 0.52 mmol) was added. The reaction mixture was stirred at room temperature for 4 h. The reaction mixture was directly concentrated and the crude residue was purified by reverse phase HPLC to provide the desired product as its TFA salt (26 mg, 0.04 mmol, 35%). LC/MS (ESI) m/z: [M+H]⁺ calcd for C₃₂H₃₃ClF₂N₅O 576.2. found 576.3.

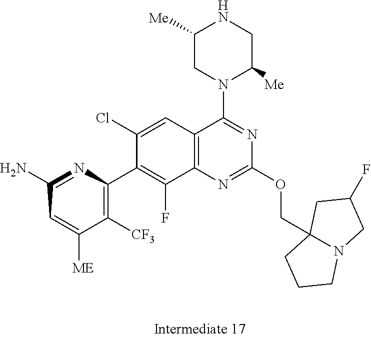

### 6-(6-chloro-4-((2R,5S)-2,5-dimethylpiperazin-1-yl)-8-fluoro-2-((2-fluorotetrahydro-1H-pyrrolizin-7a(5H)-yl)methoxy)quinazolin-7-yl)-4-methyl-5-(trifluoromethyl)pyridin-2-amine

The desired product was prepared using procedures similar to those described herein using the appropriate starting materials. ¹H NMR (400 MHz, CD₃OD) δ 7.96 (s, 1H), 6.54 (s, 1H), 5.49-5.62 (m, 1H), 4.50-4.61 (m, 2H), 4.40-4.49 (m, 1H), 3.80-3.76 (m, 8H), 3.51-3.52 (m, 2H), 3.25-3.21 (m, 1H), 3.15-3.20 (m, 2H), 2.36-2.50 (m, 2H), 2.15-2.36 (m, 1H), 1.37 (q, J=6.80 Hz, 3H), 1.29 (q, J=6.80 Hz, 3H).

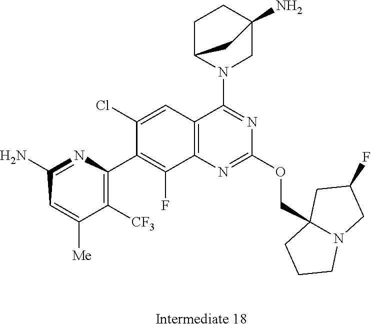

### (1R,4R)-2-(7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-2-azabicyclo[2.2.1]heptan-4-amine

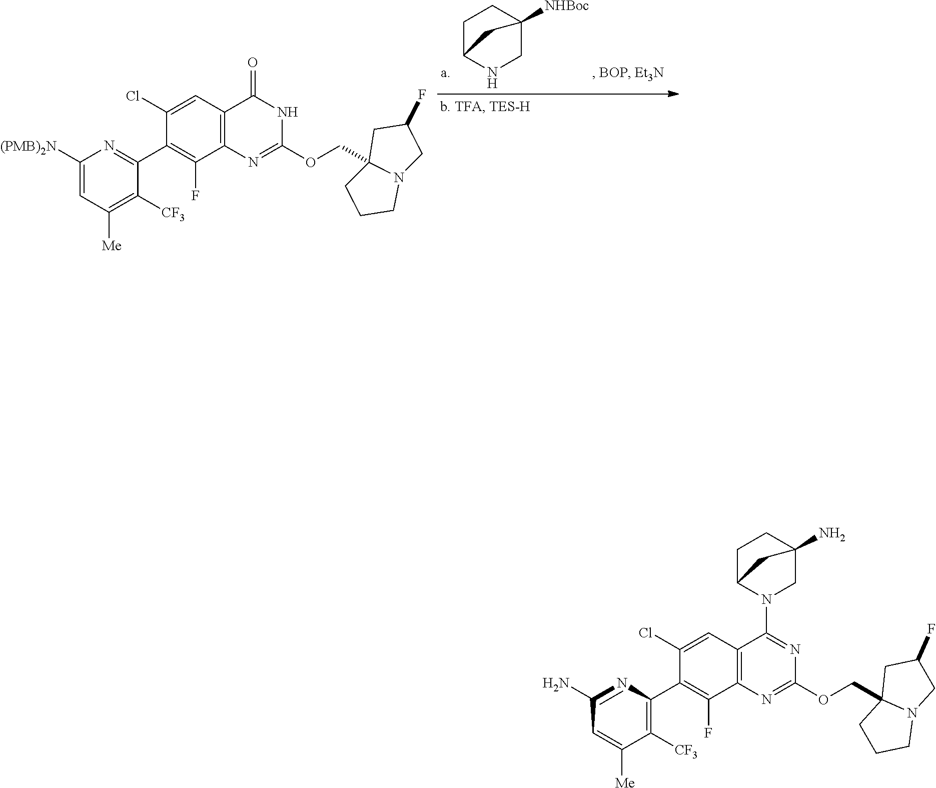

### Step 1: tert-butyl ((1R,4R)-2-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-2-azabicyclo[2.2.1]heptan-4-yl)carbamate

7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 50 mg, 0.07 mmol) and benzotriazol-1-yloxy)tris(dimethylamino)phosphonium hexafluorophosphate (57 mg, 0.13 mmol) were combined and dissolved in acetonitrile (5 mL). Triethylamine (20 μL, 0.13 mmol) was added followed by tert-butyl ((1R,4R)-2-azabicyclo[2.2.1]heptan-4-yl)carbamate (14 mg, 0.07 mmol). The reaction mixture was heated at 40° C. for 12 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was used directly in the next step, without additional purification (quantitative yield assumed). LC/MS (ESI) m/z: [M+H]⁺ calcd for C₅₀H₅₆ClF₅N₇O₅ 964.4. found 963.8.

### Step 2: (1R,4R)-2-(7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-2-azabicyclo[2.2.1]heptan-4-amine

tert-Butyl ((1R,4R)-2-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-2-azabicyclo[2.2.1]heptan-4-yl)carbamate (from Step 1, 80 mg, 0.08 mmol) was dissolved in TFA (1.5 mL), and triethylsilane (10 μL, 0.06 mmol) and water (0.05 mL) were added. The reaction mixture was heated at 40° C. for 12 h. The crude mixture was directly purified by preparative HPLC to provide the desired product (19 mg, 0.03 mmol, 33% yield). LC/MS (ESI) m/z: [M+H]⁺ calcd for C₂₉H₃₂ClF₅N₇O 624.2. found 624.2; ¹H NMR (400 MHz, CDCl₃) δ 7.79-7.64 (m, 1H), 6.44-6.28 (m, 1H), 5.38-5.17 (m, 1H), 4.92 (br d, J=1.3 Hz, 1H), 4.85-4.72 (m, 2H), 4.33 (d, J=10.3 Hz, 1H), 4.25-4.16 (m, 1H), 3.87 (br dd, J=2.6, 9.4 Hz, 1H), 3.60-3.36 (m, 2H), 3.28-3.06 (m, 1H), 2.80 (d, J=10.3 Hz, 1H), 2.72-2.54 (m, 3H), 2.47-2.29 (m, 4H), 2.23-2.09 (m, 7H), 2.00 (s, 4H), 1.94-1.81 (m, 3H), 1.82-1.69 (m, 3H), 1.62 (br d, J=9.5 Hz, 2H).

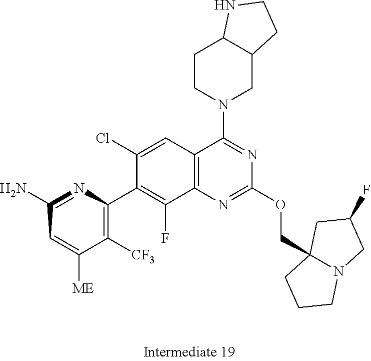

### 6-(6-chloro-8-fluoro-2-(((2R,7aR)-2-fluorotetrahydro-1H-pyrrolizin-7a(5H)-yl)methoxy)-4-(octahydro-5H-pyrrolo[3,2-c]pyridin-5-yl)quinazolin-7-yl)-4-methyl-5-(trifluoromethyl)pyridin-2-amine

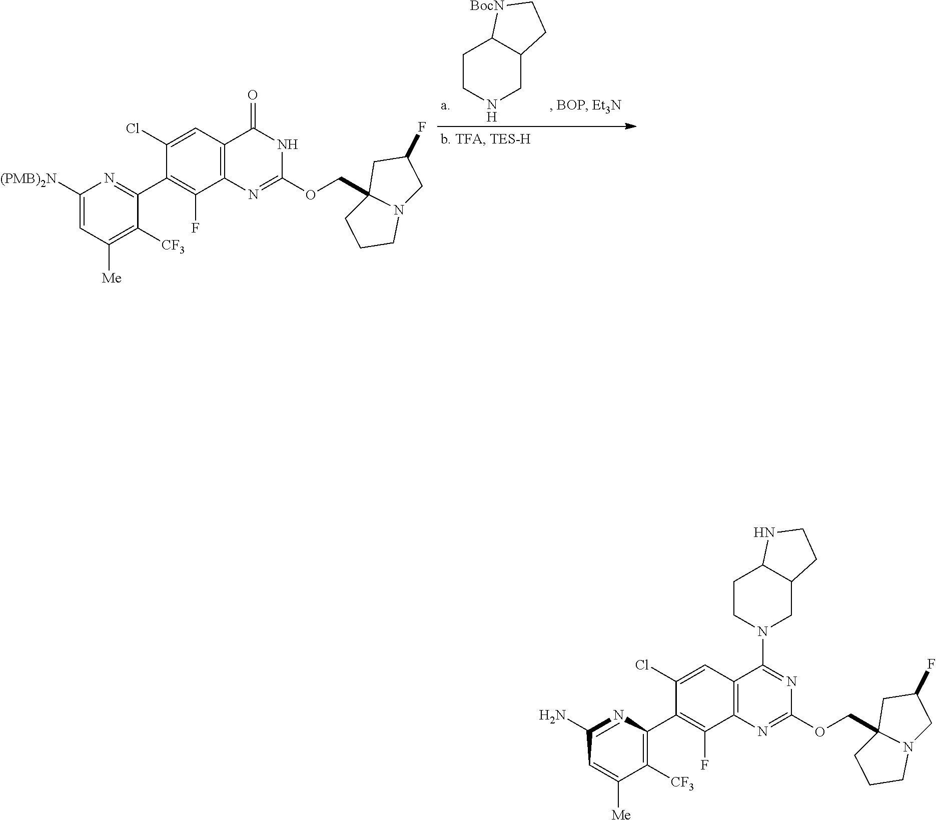

### Step 1: tert-butyl 5-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-1H-pyrrolo[3,2-c]pyridine-1-carboxylate

7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 50 mg, 0.07 mmol) and benzotriazol-1-yloxy)tris(dimethylamino)phosphonium hexafluorophosphate (43 mg, 0.10 mmol) were combined and dissolved in acetonitrile (2 mL). Triethylamine (14 μL, 0.10 mmol) was added followed by tert-butyl octahydro-1H-pyrrolo[3,2-c]pyridine-1-carboxylate (15 mg, 0.07 mmol). The reaction mixture was heated at 40° C. for 12 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was used directly in the next step, without additional purification (quantitative yield assumed). LC/MS (ESI) m/z: [M+H]⁺ calcd for C₅₁H₅₈ClF₅N₇O₅ 978.4. found 978.2.

### Step 2: 6-(6-chloro-8-fluoro-2-(((2R,7aR)-2-fluorotetrahydro-1H-pyrrolizin-7a(5H)-yl)methoxy)-4-(octahydro-5H-pyrrolo[3,2-c]pyridin-5-yl)quinazolin-7-yl)-4-methyl-5-(trifluoromethyl)pyridin-2-amine

tert-Butyl 5-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-1H-pyrrolo[3,2-c]pyridine-1-carboxylate (from Step 1, 50 mg, 0.05 mmol) was dissolved in TFA (1.5 mL), and triethylsilane (10 μL, 0.06 mmol) and water (0.05 mL) were added. The reaction mixture was heated at 40° C. for 16 h. The crude mixture was directly purified by preparative HPLC to provide the desired product (23 mg, 0.04 mmol, 68% yield). LC/MS (ESI) m/z: [M+H]⁺ calcd for C₃₀H₃₄ClF₅N₇O 638.2. found 638.2; ¹H NMR (400 MHz, CD₃OD) δ 7.93 (s, 1H), 6.63 (s, 1H), 5.28-5.49 (m, 1H), 4.36-4.44 (m, 2H), 4.08-4.19 (m, 2H), 3.70-3.90 (m, 3H), 3.41-3.48 (m, 2H), 3.13-3.33 (m, 2H), 3.13-3.16 (m, 2H), 2.79-2.82 (m, 2H), 2.47-2.47 (m, 3H), 1.93-2.05 (m, 9H).

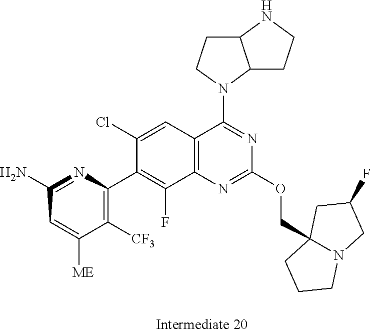

### 6-(6-chloro-8-fluoro-2-(((2R,7aR)-2-fluorotetrahydro-1H-pyrrolizin-7a(5H)-yl)methoxy)-4-(hexahydropyrrolo[3,2-b]pyrrol-1(2H)-yl)quinazolin-7-yl)-4-methyl-5-(trifluoromethyl)pyridin-2-amine

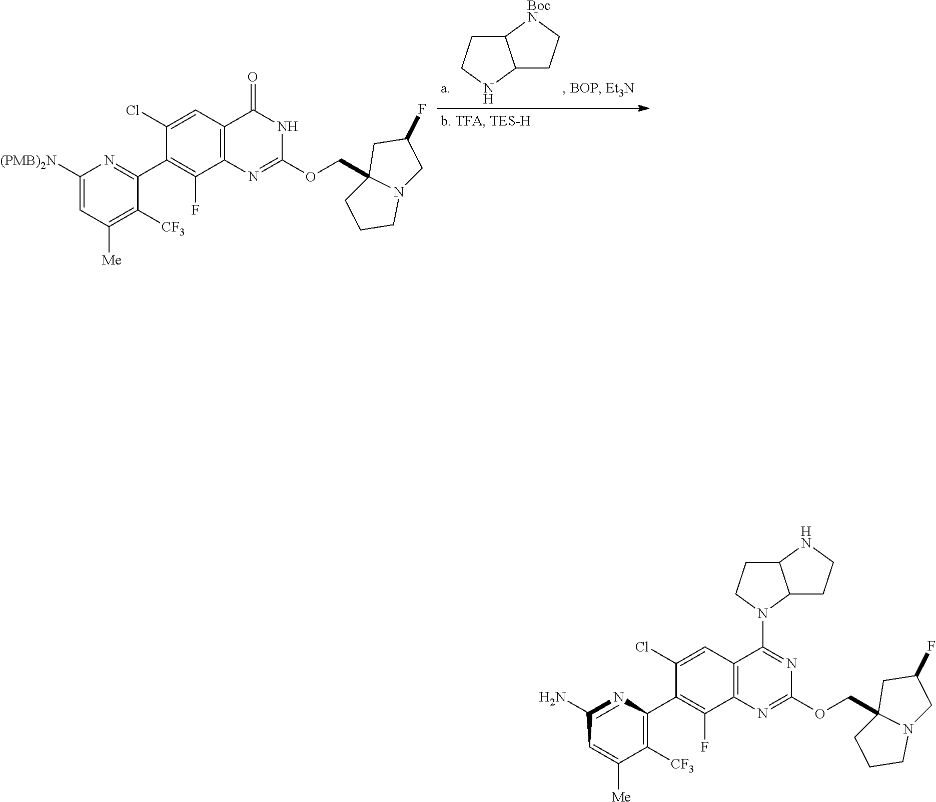

### Step 1: tert-butyl 4-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)hexahydropyrrolo[3,2-b]pyrrole-1(2H)-carboxylate

7-(6-(Bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 50 mg, 0.07 mmol) and benzotriazol-1-yloxy)tris(dimethylamino)phosphonium hexafluorophosphate (43 mg, 0.10 mmol) were combined and dissolved in acetonitrile (2 mL). Triethylamine (14 μL, 0.10 mmol) was added followed by tert-butyl hexahydropyrrolo[3,2-b]pyrrole-1(2H)-carboxylate (17 mg, 0.08 mmol). The reaction mixture was heated at 40° C. for 12 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was used directly in the next step, without additional purification (quantitative yield assumed). LC/MS (ESI) m/z: [M+H]⁻ calcd for C₅₀H₅₇ClF₅N₇O₅ 964.4. found 964.2.

### Step 2: 6-(6-chloro-8-fluoro-2-(((2R,7aR)-2-fluorotetrahydro-1H-pyrrolizin-7a(5H)-yl)methoxy)-4-(hexahydropyrrolo[3,2-b]pyrrol-1(2H)-yl)quinazolin-7-yl)-4-methyl-5-(trifluoromethyl)pyridin-2-amine

tert-Butyl 4-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)hexahydropyrrolo[3,2-b]pyrrole-1(2H)-carboxylate (from Step 1, 52 mg, 0.054 mmol) was dissolved in TFA (1.5 mL), and triethylsilane (10 μL, 0.06 mmol) and water (0.05 mL) were added. The reaction mixture was heated at 40° C. for 16 h. The crude mixture was directly purified by preparative HPLC to provide the desired product as its TFA salt (28 mg, 0.04 mmol, 70% yield). LC/MS (ESI) m/z: [M+H]⁺ calcd for C₂₉H₃₂ClF₅N₇O 624.2, found 624.2; ¹H NMR (400 MHz, CD₃OD) δ 8.19 (d, J=2.00 Hz, 1H), 6.66 (s, 1H), 5.40-5.59 (m, 2H), 4.70-4.85 (m, 2H), 4.32-4.50 (m, 3H), 4.05-4.05 (m, 1H), 3.50-3.56 (m, 5H), 2.68-2.80 (m, 1H), 2.46-2.53 (m, 9H), 2.28-2.43 (m, 3H).

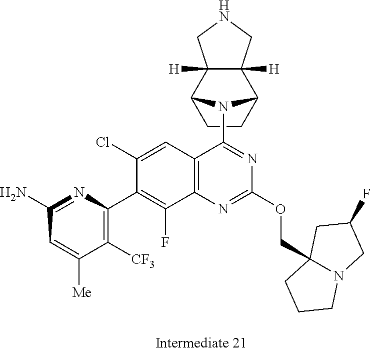

### 6-(6-chloro-8-fluoro-2-(((2R,7aR)-2-fluorotetrahydro-1H-pyrrolizin-7a(5H)-yl)methoxy)-4-((3aR,4S,7R,7aS)-octahydro-1H-4,7-epiminoisoindol-8-yl)quinazolin-7-yl)-4-methyl-5-(trifluoromethyl)pyridin-2-amine

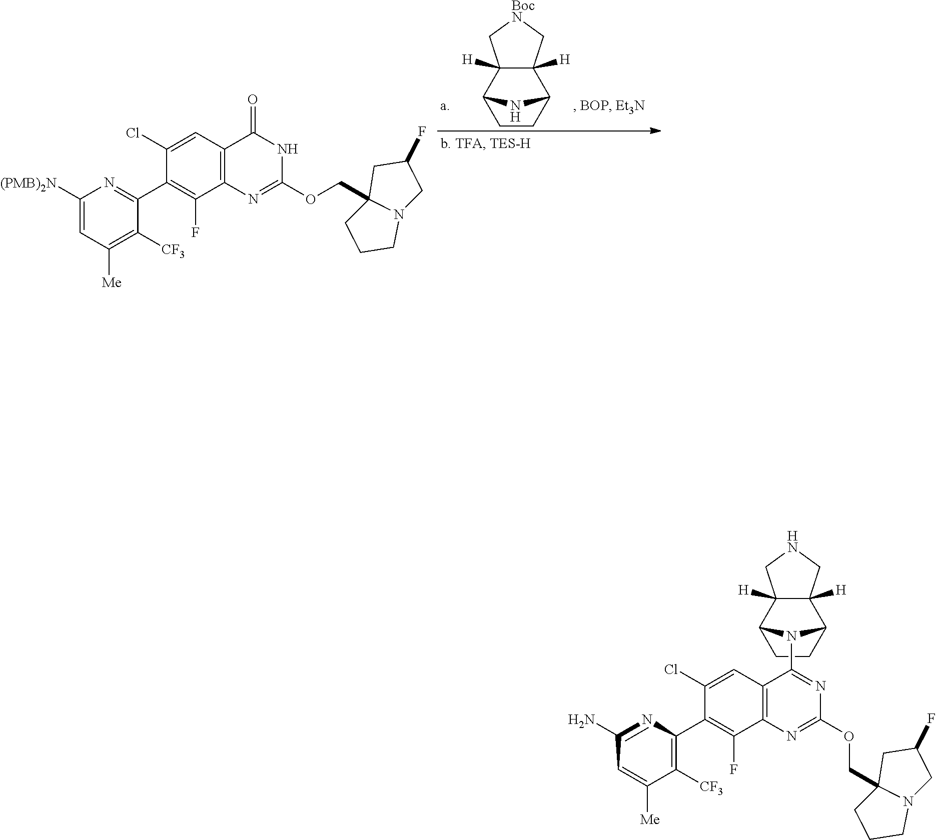

### Step 1: tert-butyl (3aR,4S,7R,7aS)-8-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-2H-4,7-epiminoisoindole-2-carboxylate

7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 50 mg, 0.07 mmol) and benzotriazol-1-yloxy)tris(dimethylamino)phosphonium hexafluorophosphate (57 mg, 0.13 mmol) were combined and dissolved in acetonitrile (5 mL). Triethylamine (18 μL, 0.13 mmol) was added followed by tert-butyl (3aR,4S,7R,7aS)-octahydro-2H-4,7-epiminoisoindole-2-carboxylate (15 mg, 0.07 mmol). The reaction mixture was heated at 40° C. for 12 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was used directly in the next step, without additional purification (quantitative yield assumed). LC/MS (ESI) m/z: [M+H]⁺ calcd for C₅₂H₅₈ClF₅N₇O₅ 990.4. found 990.0.

### Step 2: 6-(6-chloro-8-fluoro-2-(((2R,7aR)-2-fluorotetrahydro-1H-pyrrolizin-7a(5H)-yl)methoxy)-4-((3aR,4S,7R,7aS)-octahydro-1H-4,7-epiminoisoindol-8-yl)quinazolin-7-yl)-4-methyl-5-(trifluoromethyl)pyridin-2-amine

tert-Butyl (3aR,4S,7R,7aS)-8-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-2H-4,7-epiminoisoindole-2-carboxylate (from Step 1, 80 mg, 0.08 mmol) was dissolved in TFA (1.5 mL), and triethylsilane (10 μL, 0.06 mmol) and water (0.05 mL) were added. The reaction mixture was heated at 40° C. for 16 h. The crude mixture was directly purified by preparative HPLC to provide the desired product (13 mg, 0.02 mmol, 24% yield). LC/MS (ESI) m/z: [M+H]⁺ calcd for C₃₁H₃₄ClF₅N₇O 650.2. found 649.8; ¹H NMR (400 MHz, CDCl₃) δ 7.70-7.57 (m, 1H), 6.40 (s, 1H), 5.43-5.16 (m, 2H), 4.74 (br s, 3H), 4.40-4.13 (m, 2H), 3.53-3.33 (m, 1H), 3.15-2.98 (m, 3H), 2.95-2.77 (m, 3H), 2.75-2.69 (m, 1H), 2.67-2.53 (m, 2H), 2.41 (d, J=1.5 Hz, 2H), 2.17-2.07 (m, 2H), 2.00 (s, 2H), 1.90-1.84 (m, 1H), 1.94-1.62 (m, 2H).

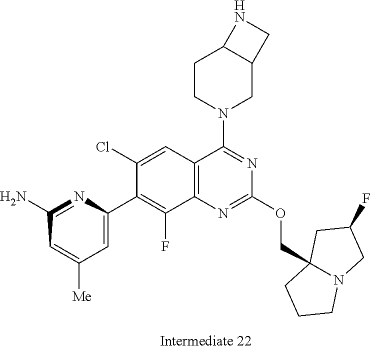

### 6-(4-(3,7-diazabicyclo[4.2.0]octan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine

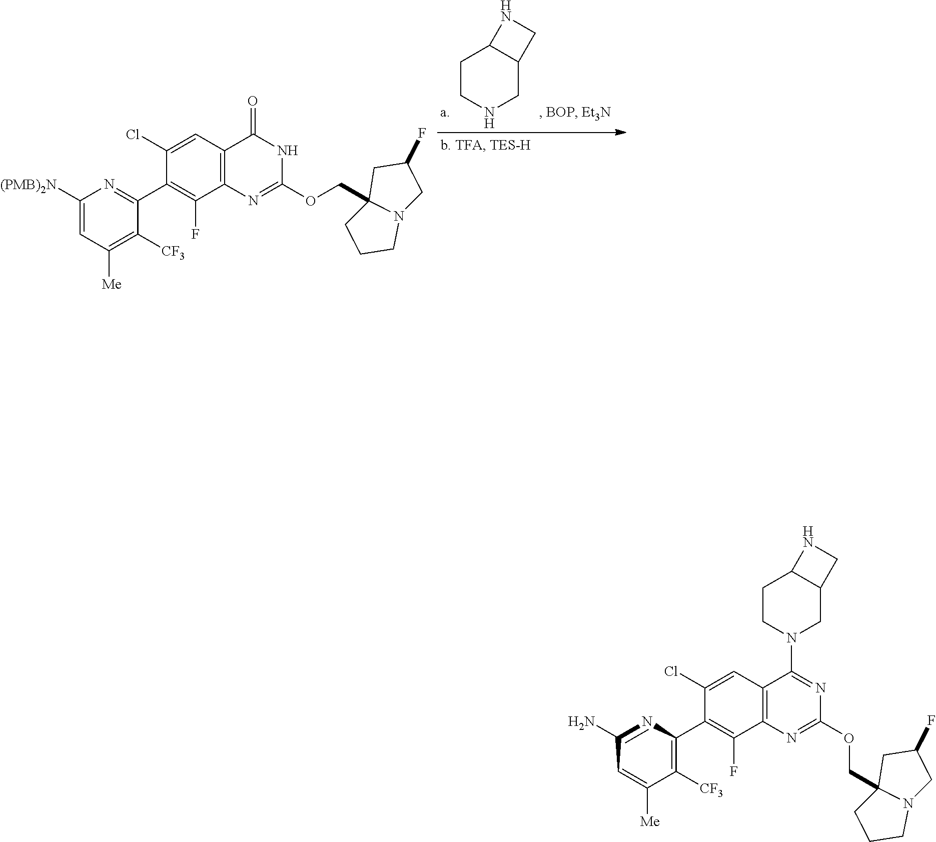

### Step 1: tert-butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,7-diazabicyclo[4.2.0]octane-7-carboxylate

7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 50 mg, 0.07 mmol) and benzotriazol-1-yloxy)tris(dimethylamino)phosphonium hexafluorophosphate (43 mg, 0.10 mmol) were combined and dissolved in acetonitrile (5 mL). Triethylamine (14 μL, 0.10 mmol) was added followed by tert-butyl 3,7-diazabicyclo[4.2.0]octane-7-carboxylate (17 mg, 0.08 mmol) The reaction mixture was heated at 40° C. for 12 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was used directly in the next step, without additional purification (quantitative yield assumed). LC/MS (ESI) m/z: [M+H]⁺ calcd for C₅₀H₅₆ClF₅N₇O₅ 964.4. found 964.2.

### Step 2: 6-(4-(3,7-diazabicyclo[4.2.0]octan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine

tert-Butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,7-diazabicyclo[4.2.0]octane-7-carboxylate (from Step 1, 54 mg, 0.06 mmol) was dissolved in TFA (1.5 mL), and triethylsilane (10 μL, 0.06 mmol) and water (0.05 mL) were added. The reaction mixture was heated at 40° C. for 16 h. The crude mixture was directly purified by preparative HPLC to provide the desired product (12 mg, 0.02 mmol, 33% yield). LC/MS (ESI) m/z: [M+H]⁺ calcd for C₂₉H₃₂ClF₅N₇O 624.2. found 624.2; ¹H NMR (400 MHz, CD₃OD) δ 8.07-8.09 (m, 1H), 6.63 (d, J=0.80 Hz, 1H), 5.31-5.44 (m, 1H), 4.70-4.90 (m, 1H), 4.37-4.50 (m, 3H), 3.94-4.17 (m, 4H), 3.32-3.35 (m, 2H), 3.19-3.22 (m, 1H), 2.86-2.89 (m, 2H), 2.38-2.55 (m, 5H), 2.16-2.19 (m, 1H), 1.95-2.03 (m, 4H).

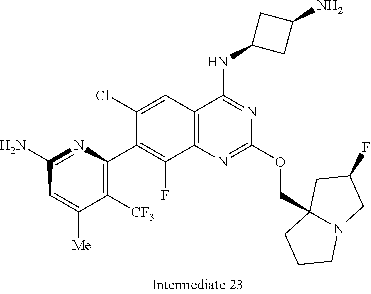

### (1s,3S)-N1-((R)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)cyclobutane-1,3-diamine

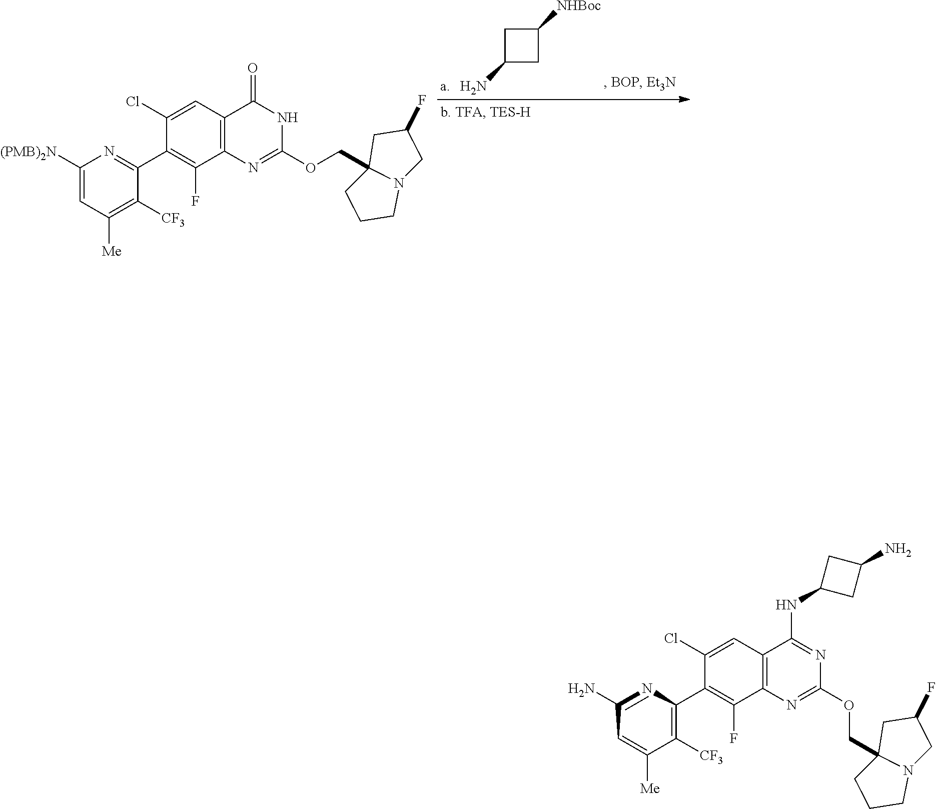

### Step 1: tert-butyl ((1S,3s)-3-((7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)amino)cyclobutyl)carbamate

7-(6-(Bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 70 mg, 0.09 mmol) and benzotriazol-1-yloxy)tris(dimethylamino)phosphonium hexafluorophosphate (60 mg, 0.14 mmol) were combined and dissolved in acetonitrile (5 mL). Triethylamine (20 μL, 0.14 mmol) was added followed by tert-butyl ((1s,3s)-3-aminocyclobutyl)carbamate (17 mg, 0.09 mmol). The reaction mixture was heated at 40° C. for 12 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was used directly in the next step, without additional purification (quantitative yield assumed). LC/MS (ESI) m/z: [M+H]⁺ calcd for C₄₈H₅₄ClF₅N₇O₅ 938.4. found 938.4.

### Step 2: (1s,3S)-N1-((R)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)cyclobutane-1,3-diamine

tert-Butyl ((1S,3s)-3-((7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)amino)cyclobutyl)carbamate (73 mg, 0.078 mmol) was dissolved in TFA (1.5 mL), and triethylsilane (10 μL, 0.06 mmol) and water (0.05 mL) were added. The reaction mixture was heated at 40° C. for 16 h. The crude mixture was directly purified by preparative HPLC to provide the desired product as the AcOH salt (17 mg, 0.03 mmol, 34% yield). LC/MS (ESI) m/z: [M+H]⁺ calcd for C₂₇H₃₀ClF₅N₇O₅ 598.2. found 598.2. ¹H NMR (400 MHz, CD₃OD) δ 8.26-8.03 (m, 1H), 6.62 (s, 1H), 5.59-5.23 (m, 1H), 4.56-4.06 (m, 4H), 3.64-3.54 (m, 1H), 3.54-3.47 (m, 1H), 3.24-3.14 (m, 1H), 3.09-2.93 (m, 3H), 2.87 (td, J=5.5, 11.0 Hz, 1H), 2.64-2.52 (m, 1H), 2.47 (d, J=1.4 Hz, 3H), 2.34-2.20 (m, 2H), 2.19-2.09 (m, 1H), 2.08-1.97 (m, 3H), 1.96-1.95 (m, 3H), 1.92-1.82 (m, 1H).

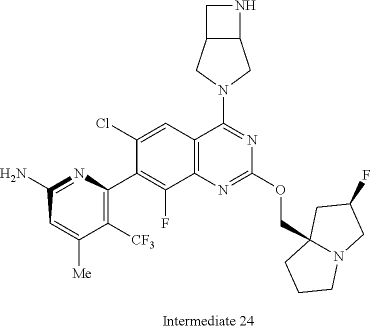

### 6-(4-(3,6-diazabicyclo[3.2.0]heptan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine

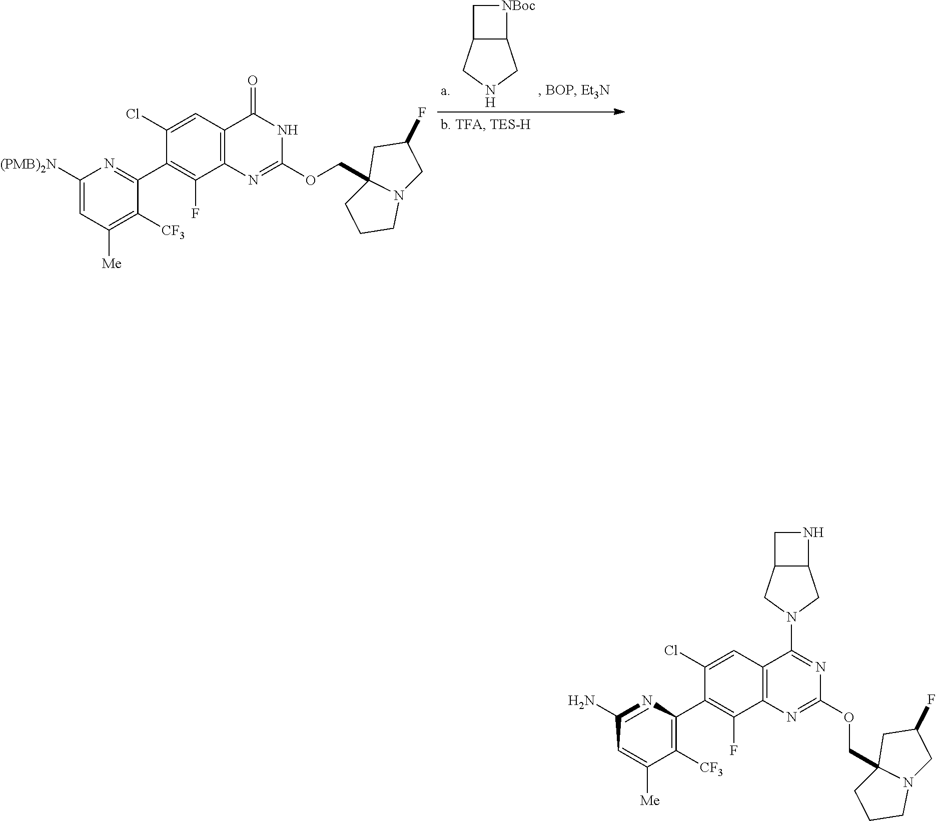

### Step 1: tert-butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.2.0]heptane-6-carboxylate

7-(6-(Bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 60 mg, 0.08 mmol) and benzotriazol-1-yloxy)tris(dimethylamino)phosphonium hexafluorophosphate (52 mg, 0.12 mmol) were combined and dissolved in acetonitrile (5 mL). Triethylamine (17 μL, 0.12 mmol) was added followed by tert-butyl 3,6-diazabicyclo[3.2.0]heptane-6-carboxylate (31 mg, 0.16 mmol). The reaction mixture was heated at 40° C. for 12 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was used directly in the next step, without additional purification (quantitative yield assumed). LC/MS (ESI) m/z: [M+H]⁺ calcd for C₄₉H₅₄ClF₅N₇O₅ 950.4. found 950.4.

### Step 2: 6-(4-(3,6-diazabicyclo[3.2.0]heptan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine

tert-Butyl 3-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.2.0]heptane-6-carboxylate (from Step 1, 60 mg, 0.063 mmol) was dissolved in TFA (1.5 mL), and triethylsilane (10 μL, 0.06 mmol) and water (0.05 mL) were added. The reaction mixture was heated at 40° C. for 16 h. The crude mixture was directly purified by preparative HPLC to provide the desired product (17 mg, 0.03 mmol, 41% yield). LC/MS (ESI) m/z: [M+H]⁺ calcd for C₂₈H₃₀ClF₅N₇O₅ 610.2. found 610.2; ¹H NMR (400 MHz, CD₃OD) δ 8.22-8.23 (m, 1H), 6.63 (s, 1H), 5.37 (d, J=52.80 Hz, 1H), 4.64-4.72 (m, 1H), 4.42-4.50 (m, 2H), 3.95-4.09 (m, 3H), 3.50-3.55 (m, 3H), 3.15-3.23 (m, 2H), 2.88-2.89 (m, 1H), 2.47-2.51 (m, 3H), 2.09-2.21 (m, 4H), 1.93-2.00 (m, 4H).

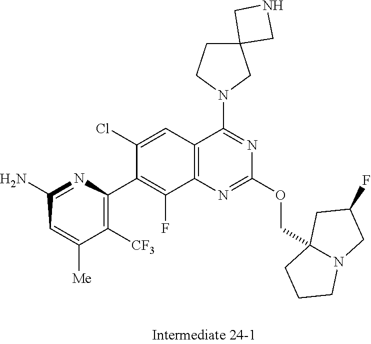

### 6-(6-chloro-8-fluoro-2-(((2R,7aR)-2-fluorotetrahydro-1H-pyrrolizin-7a(5H)-yl)methoxy)-4-(2,6-diazaspiro[3.4]octan-6-yl)quinazolin-7-yl)-4-methyl-5-(trifluoromethyl)pyridin-2-amine

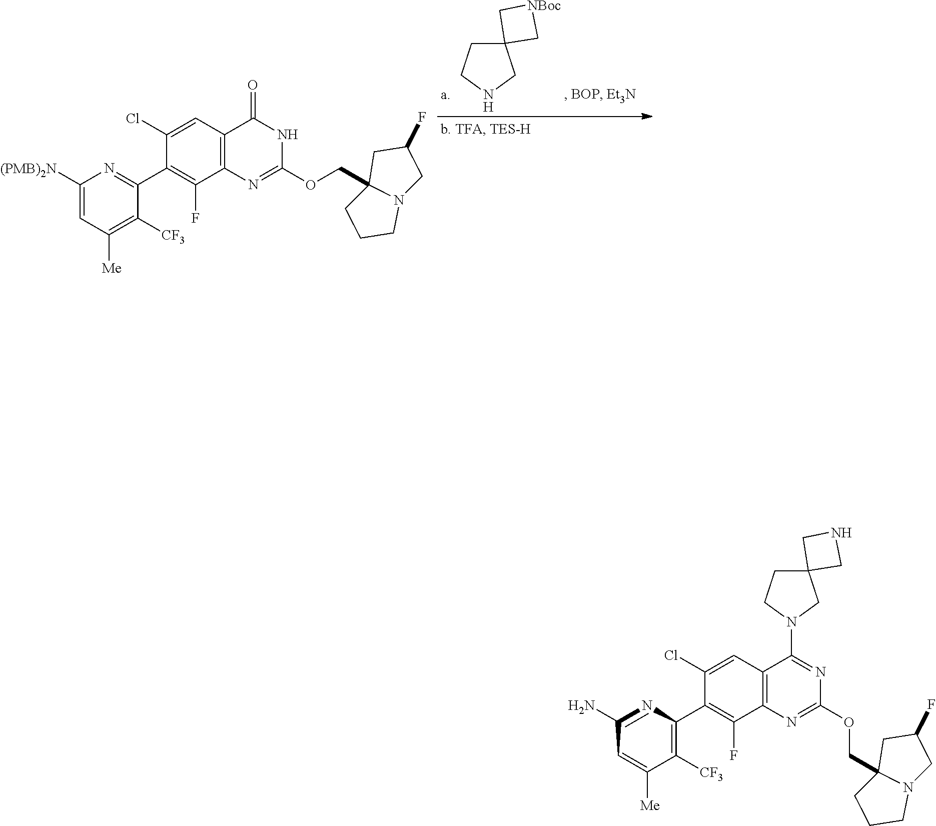

### Step 1: tert-butyl 6-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-2,6-diazaspiro[3.4]octane-2-carboxylate

7-(6-(Bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 50 mg, 0.07 mmol) and benzotriazol-1-yloxy)tris(dimethylamino)phosphonium hexafluorophosphate (29 mg, 0.07 mmol) were combined and dissolved in acetonitrile (5 mL). Triethylamine (10 μL, 0.07 mmol) was added followed by tert-butyl 2,6-diazaspiro[3.4]octane-2-carboxylate (13.78 mg, 0.07 mmol) The reaction mixture was heated at 40° C. for 12 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was used directly in the next step, without additional purification (quantitative yield assumed). LC/MS (ESI) m/z: [M+H]⁺ calcd for C₅₀H₅₆ClF₅N₇O₅ 964.4. found 964.4.

### Step 2: 6-(6-chloro-8-fluoro-2-(((2R,7aR)-2-fluorotetrahydro-1H-pyrrolizin-7a(5H)-yl)methoxy)-4-(2,6-diazaspiro[3.4]octan-6-yl)quinazolin-7-yl)-4-methyl-5-(trifluoromethyl)pyridin-2-amine

tert-Butyl 6-(7-(6-(bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-2,6-diazaspiro[3.4]octane-2-carboxylate (from Step 1, 50 mg, 0.052 mmol was dissolved in TFA (1.5 mL), and triethylsilane (10 μL, 0.06 mmol) and water (0.05 mL) were added. The reaction mixture was heated at 40° C. for 16 h. The crude mixture was directly purified by preparative HPLC to provide the desired product as the formic acid salt (8 mg, 0.01 mmol, 20% yield). LC/MS (ESI) m/z: [M+H]⁺ calcd for C₂₉H₃₂ClF₅N₇O₅ 624.2. found 624.2.

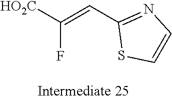 [SMILES: O=C(O)/C(F)=C/c1nccs1]

### (Z)-2-fluoro-3-(thiazol-2-yl)acrylic acid

### Step 1: Preparation of ethyl (E)-2-fluoro-3-(thiazol-2-yl)acrylate

Ethyl 2-(diethoxyphosphoryl)-2-fluoroacetate (1.1 g, 4.5 mmol) was dissolved in THE (25 mL). The solution was cooled to 0° C. Sodium hydride (60% dispersion in mineral oil, 0.18 g, 4.5 mmol) was added portionwise as a solid. The reaction mixture was stirred for 10 min and thiazole-2-carbaldehyde (0.51 g, 4.5 mmol) was added. The reaction mixture was allowed to warm to room temperature and was stirred for 1 h. The mixture was quenched by addition of saturated aqueous ammonium chloride solution (20 mL). The solution was diluted with water (20 mL) and EtOAc (150 mL). The layers were separated and the aqueous phase was further extracted with EtOAc (2×50 mL). The combined organic layers were dried over magnesium sulfate, filtered, and concentrated. The crude residue was purified by column chromatogrpahy (0→60% acetone/hexanes) to provide ethyl (E)-2-fluoro-3-(thiazol-2-yl)acrylate (10:1 E/Z as judged by ¹H NMR, 620 mg, 3.1 mmol, 68% yield). LC/MS (ESI) m/z: [M+H]⁺ calcd for C₈H₉FNO₂S 202.0; found 202.2; ¹H NMR (500 MHz, CDCl₃) δ 7.94 (d, J=3.2 Hz, 1H), 7.52 (d, J=3.2 Hz, 1H), 7.34 (d, J=22.2 Hz, 1H), 4.43 (q, J=7.2 Hz, 2H), 1.41 (t, J=7.2 Hz, 3H).

### Step 2: Preparation of ethyl (Z)-2-fluoro-3-(thiazol-2-yl)acrylate

Ethyl (E)-2-fluoro-3-(thiazol-2-yl)acrylate (620 mg, 3.1 mmol) was dissolved in toluene (15 mL) and iodine (39 mg, 0.15 mmol) was added. The reaction mixture was heated at 100° C. for 7 days. The solution was concentrated and purified by column chromatography (0→100% EtOAc/hexanes) to provide ethyl (Z)-2-fluoro-3-(thiazol-2-yl)acrylate (509 mg, 2.5 mmol, 82% yield). LC/MS (ESI) m/z: [M+H]⁺ calcd for C₈H₉FNO₂S 202.0. found 202.0; ¹H NMR (500 MHz, CDCl₃) δ 7.96 (dd, J=3.2, 2.6 Hz, 1H), 7.57 (d, J=3.2 Hz, 1H), 7.43 (dd, J=33.3, 0.8 Hz, 1H), 4.38 (q, J=7.2 Hz, 2H), 1.39 (t, J=7.1 Hz, 3H).

### Step 3: Preparation of (Z)-2-fluoro-3-(thiazol-2-yl)acrylic acid

Ethyl (Z)-2-fluoro-3-(thiazol-2-yl)acrylate (510 mg, 2.5 mmol) was dissolved in MeOH (15 mL). The solution was cooled to 0° C. and sodium hydroxide solution (1.0 M, 2.5 mL, 2.5 mmol) was added. The reaction mixture was stirred for 5 h. The solution was concentrated to remove the methanol. Additional water (1.5 mL) was added and the aqueous solution was cooled to 0° C. HCl solution (1.0 M, 2.5 mL, 2.5 mmol) was added dropwise. After 10 min, a white solid precipitated. The solid was collected by filtration and was washed with MeCN. The solid was dried under vacuum to provide (Z)-2-fluoro-3-(thiazol-2-yl)acrylic acid (337 mg, 1.9 mmol, 77% yield) as a white solid. LC/MS (ESI) m/z: [M+H]⁺ calcd for C₆H₅FNO₂S 174.0. found 173.8; ¹H NMR (500 MHz, DMSO-d₆) δ 8.04 (s, 2H), 7.29 (d, J=34.5 Hz, 1H).

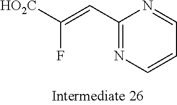 [SMILES: O=C(O)/C(F)=C/c1ncccn1]

### (Z)-2-fluoro-3-(pyrimidin-2-yl)acrylic acid

### Step 1: Preparation of ethyl (E)-2-fluoro-3-(pyrimidin-2-yl)acrylate

Ethyl 2-(diethoxyphosphoryl)-2-fluoroacetate (1.6 g, 6.6 mmol) was dissolved in THE (50 mL). The solution was cooled to 0° C. Sodium hydride (60% dispersion in mineral oil, 0.26 g, 6.6 mmol) was added portionwise as a solid. The reaction mixture was stirred for 10 min and pyrimidine-2-carbaldehyde (0.71 g, 6.6 mmol) was added. The reaction mixture was allowed to warm to room temperature and was stirred for 1 h. The mixture was quenched by addition of saturated aqueous ammonium chloride solution (20 mL). The solution was diluted with water (20 mL) and EtOAc (150 mL). The layers were separated and the aqueous phase was further extracted with EtOAc (2×50 mL). The combined organic layers were dried over magnesium sulfate, filtered, and concentrated. The crude residue was purified by column chromatogrpahy (0→90% EtOAc/hexanes) to provide ethyl (E)-2-fluoro-3-(pyrimidin-2-yl)acrylate (5:1 E/Z as judged by 1H NMR, 592 mg, 3.0 mmol, 46% yield). LC/MS (ESI) m/z: [M+H]⁺ calcd for C₉H₁₀FN₂O₂ 197.2; found 197.1; ¹H NMR (500 MHz, CDCl₃) δ 8.70 (d, J=4.9 Hz, 2H), 7.18 (t, J=4.9 Hz, 1H), 6.77 (d, J=17.5 Hz, 1H), 4.28 (q, J=7.2 Hz, 3H), 1.39 (t, J=7.2 Hz, 3H).

### Step 2: Preparation of ethyl (Z)-2-fluoro-3-(pyrimidin-2-yl)acrylate

Ethyl (E)-2-fluoro-3-(pyrimidin-2-yl)acrylate (592 mg, 3.0 mmol) was dissolved in toluene (15 mL) and iodine (38 mg, 0.15 mmol) was added. The reaction mixture was heated at 100° C. for 7 days. The solution was concentrated and purified by column chromatography (0→100% EtOAc/hexanes) to provide ethyl (Z)-2-fluoro-3-(pyrimidin-2-yl)acrylate (253 mg, 1.3 mmol, 43% yield). LC/MS (ESI) m/z: [M+H]⁻ calcd for C₉H₁₀FN₂O₂ 197.2. found 196.6; ¹H NMR (500 MHz, CDCl₃) δ 8.83 (d, J=4.9 Hz, 2H), 7.21 (t, J=4.9 Hz, 1H), 7.13 (d, J=30.8 Hz, 1H), 4.38 (q, J=7.2 Hz, 2H), 1.39 (t, J=7.2 Hz, 3H).

### Step 3: Preparation of (Z)-2-fluoro-3-(pyrimidin-2-yl)acrylic acid

Ethyl (Z)-2-fluoro-3-(pyrimidin-2-yl)acrylate (253 mg, 1.3 mmol) was dissolved in MeOH (10 mL). The solution was cooled to 0° C. and sodium hydroxide solution (1.0 M, 1.3 mL, 1.3 mmol) was added. The reaction mixture was stirred for 2 h. The solution was concentrated to remove the methanol. Additional water (1.0 mL) was added and the aqueous solution was cooled to 0° C. HCl solution (1.0 M, 2.5 mL, 2.5 mmol) was added dropwise. After 10 min, a white solid precipitated. The solid was collected by filtration and dried under vacuum to provide (Z)-2-fluoro-3-(pyrimidin-2-yl)acrylic acid (190 mg, 1.1 mmol, 88% yield) as a white solid. LC/MS (ESI) m/z: [M+H]⁺ calcd for C₇H₆FN₂O₂ 169.1. found 168.8; ¹H NMR (500 MHz, DMSO-d₆) δ 8.90 (d, J=4.9 Hz, 2H), 7.46 (t, J=4.9 Hz, 1H), 6.94 (d, J=31.5 Hz, 1H).

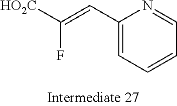 [SMILES: O=C(O)/C(F)=C/c1ccccn1]

### (Z)-2-fluoro-3-(pyridin-2-yl)acrylic acid

### Step 1: Preparation of ethyl (E)-2-fluoro-3-(pyridin-2-yl)acrylate

Ethyl 2-(diethoxyphosphoryl)-2-fluoroacetate (1.5 g, 6.2 mmol) was dissolved in THE (31 mL). The solution was cooled to 0° C. Sodium hydride (60% dispersion in mineral oil, 0.25 g, 6.2 mmol) was added portionwise as a solid. The reaction mixture was stirred for 10 min and picolinaldehyde (0.66 g, 6.2 mmol) was added. The reaction mixture was allowed to warm to room temperature and was stirred for 1 h. The mixture was quenched by addition of saturated aqueous ammonium chloride solution (15 mL). The solution was diluted with water (20 mL) and EtOAc (100 mL). The layers were separated and the aqueous phase was further extracted with EtOAc (2×50 mL). The combined organic layers were dried over magnesium sulfate, filtered, and concentrated. The crude residue was purified by column chromatogrpahy (0→100% EtOAc/hexanes) to provide ethyl (E)-2-fluoro-3-(pyridin-2-yl)acrylate (3:1 E/Z mixture as judged by ¹H NMR, 910 mg, 4.6 mmol, 75% yield). LC/MS (ESI) m/z: [M+H]⁺ calcd for C₁₀H₁₁FNO₂ 196.1. found 196.1; E isomer reported: ¹H NMR (500 MHz, CDCl₃) δ 8.59 (dd, J=4.9, 1.8 Hz, 1H), 7.67 (ddd, J=7.9, 7.6, 1.8 Hz, 1H), 7.56 (d, J=7.9 Hz, 1H), 7.21 (dd, J=7.6, 4.9 Hz, 1H), 6.90 (d, J=20.4 Hz, 1H), 4.25 (q, J=7.2 Hz, 2H), 1.23 (t, J=7.1 Hz, 3H).

### Step 2: Preparation of ethyl (Z)-2-fluoro-3-(pyridin-2-yl)acrylate

Ethyl (E)-2-fluoro-3-(pyridin-2-yl)acrylate (420 mg, 2.1 mmol) was dissolved in toluene (10 mL) and iodine (27 mg, 0.15 mmol) was added. The reaction mixture was heated at 100° C. for 7 days. The solution was concentrated and purified by column chromatography (0→100% EtOAc/hexanes) to provide ethyl (Z)-2-fluoro-3-(pyridin-2-yl)acrylate (240 mg, 1.2 mmol, 58% yield). LC/MS (ESI) m/z: [M+H]⁺ calcd for C₁₀H₁₁FNO₂ 196.1. found 196.1; ¹H NMR (500 MHz, CDCl₃) δ 8.66 (ddd, J=5.0, 1.8, 0.8 Hz, 1H), 7.88 (ddd, J=8.0, 1.2, 0.8 Hz, 1H), 7.75 (ddd, J=8.0, 7.8, 1.8 Hz, 1H), 7.25 (ddd, J=7.8, 5.0, 1.2 Hz, 1H), 7.14 (d, J=34.9 Hz, 1H), 4.36 (q, J=7.2 Hz, 2H), 1.38 (t, J=7.2 Hz, 3H).

### Step 3: Preparation of (Z)-2-fluoro-3-(pyridin-2-yl)acrylic acid

Ethyl (Z)-2-fluoro-3-(pyridin-2-yl)acrylate (415 mg, 2.1 mmol) was dissolved in MeOH (15 mL). The solution was cooled to 0° C. and sodium hydroxide solution (1.0 M, 2.1 mL, 2.1 mmol) was added. The reaction mixture was stirred for 5 h. The solution was concentrated to remove the methanol. Additional water (2.0 mL) was added and the aqueous solution was cooled to 0° C. HCl solution (1.0 M, 2.1 mL, 2.1 mmol) was added dropwise. After 10 min, a white solid precipitated. The solid was collected by filtration and was washed with Et20. The solid was dried under vacuum to provide (Z)-2-fluoro-3-(pyridin-2-yl)acrylic acid (235 mg, 1.4 mmol, 66% yield) as a white solid. LC/MS (ESI) m/z: [M+H]⁺ calcd for C₈H₇FNO₂ 168.0. found 167.8. ¹H NMR (500 MHz, DMSO-d₆) δ 8.66 (ddd, J=4.8, 1.8, 0.8 Hz, 1H), 7.89 (ddd, J=8.0, 7.5, 1.8 Hz, 1H), 7.81 (ddd, J=8.0, 1.1, 0.8 Hz, 1H), 7.40 (dd, J=7.5, 4.8, 1.1 Hz, 1H), 6.97 (d, J=35.3 Hz, 1H).

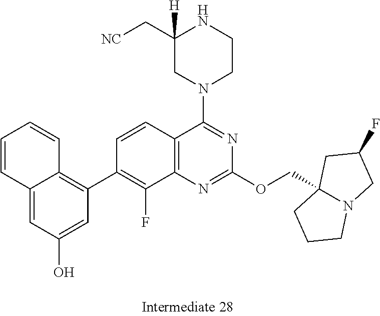 [SMILES: N#CC[C@H]1CN(c2nc(OC[C@@]34CCCN3C[C@H](F)C4)nc3c(F)c(-c4cc(O)cc5ccccc45)ccc23)CCN1]

### 2-((S)-4-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)piperazin-2-yl)acetonitrile

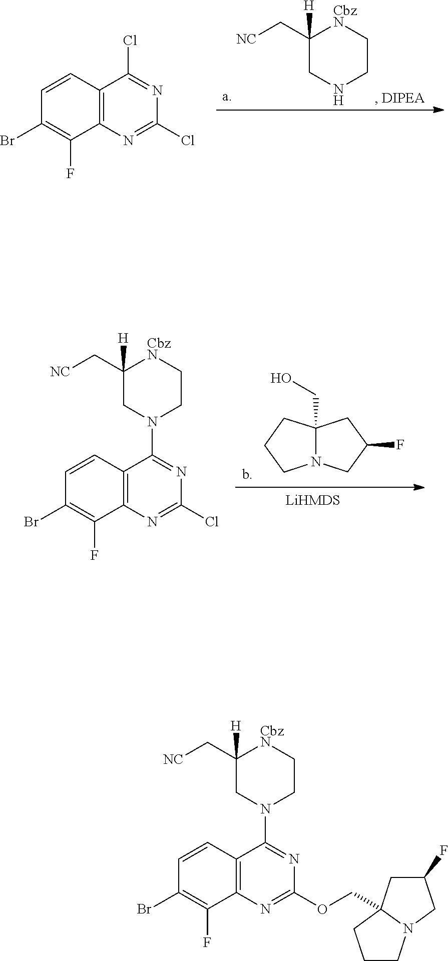

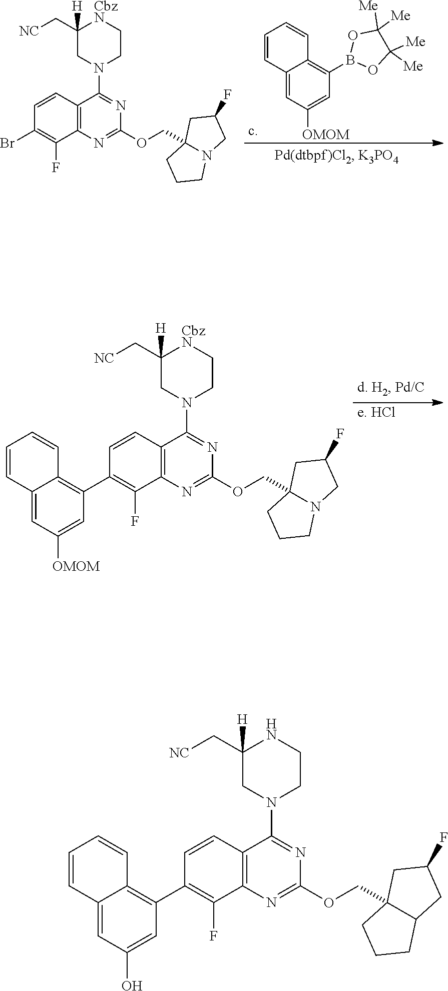

### Step 1: Benzyl (S)-4-(7-bromo-2-chloro-8-fluoroquinazolin-4-yl)-2-(cyanomethyl)piperazine-1-carboxylate

7-Bromo-2,4-dichloro-8-fluoroquinazoline (500 mg, 1.7 mmol) was dissolved in dioxane (15 ml) and DIPEA (0.89 mL, 5.1 mmol) was added followed by benzyl (S)-2-(cyanomethyl)piperazine-1-carboxylate (440 mg, 1.7 mmol). The reaction mixture was stirred at room temperature for 2 h. The mixture was diluted with EtOAc (20 mL) and was quenched by addition of a NaOH solution (1.0 M, 2 mL). The layers were separated and the organic phase was washed with brine (10 mL). The combined aqueous layers were back extracted with EtOAc (2×20 mL). The combined organic layers were dried over magnesium sulfate, filtered, and concentrated. The crude residue was purified by column chromatography (5→50% EtOAc/hexanes) to provide the desired product (760 mg, 1.5 mmol, 87% yield) as a white solid. LC/MS (ESI) m/z: [M+H]⁺ calcd for C₂₂H₁₉BrClFN₅O₂ 518.0. found 517.9; NMR: ¹H NMR (500 MHz, CD₃OD) δ 7.80 (dd, J=9.1, 1.5 Hz, 1H), 7.68 (dd, J=9.1, 6.5 Hz, 1H), 7.45-7.28 (m, 5H), 5.24-5.12 (m, 2H), 4.79-4.71 (m, 1H), 4.50-4.34 (m, 2H), 4.14-4.08 (m, 1H), 3.81-3.70 (m, 1H), 3.69-3.61 (m, 1H), 3.59-3.46 (m, 1H), 3.04-2.88 (m, 2H).

### Step 2: Benzyl (S)-4-(7-bromo-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate

Benzyl (S)-4-(7-bromo-2-chloro-8-fluoroquinazolin-4-yl)-2-(cyanomethyl)piperazine-1-carboxylate (from Step 1, 250 mg, 0.48 mmol) and ((2R,7aS)-2-fluorotetrahydro-1H-pyrrolizin-7a(5H)-yl)methanol, HCl salt (commercially available, or prepared according to the procedure described in WO 2022/031678, 104 mg, 0.53 mmol), were suspended in THE (8 mLA solution of LiHMDS (1.0 M in THF, 1.1 mL, 1.1 mmol) was added dropwise and the reaction mixture was heated at 65° C. for 16 h. The mixture was diluted with EtOAc (20 mL) and was washed with water (10 mL) and brine (10 mL). The organic phase was dried over magnesium sulfate, filtered, and concentrated. The crude residue was purified by column chromatogrpahy (0→8% MeOH/DCM) to provide the desired product (184 mg, 0.29 mmol, 60% yield) as a white solid. LC/MS (ESI) m/z: [M+H]⁺ calcd for C₃₀H₃₂BrF₂N₆O₃ 641.2. found 641.1.

### Step 3: Benzyl (S)-2-(cyanomethyl)-4-(8-fluoro-2-(((2R,7aS)-2-fluorotetrahydro-1H-pyrrolizin-7a(5H)-yl)methoxy)-7-(3-(methoxymethoxy)naphthalen-1-yl)quinazolin-4-yl)piperazine-1-carboxylate

Benzyl (S)-4-(7-bromo-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate (from Step 2, 80 mg, 0.125 mmol), 2-(3-(methoxymethoxy)naphthalen-1-yl)-4,4,5,5-tetramethyl-1,3,2-dioxaborolane (39 mg, 0.125 mmol), and potassium phosphate tribasic solution (2.0 M, 0.19 mL, 0.375 mmol) were dissolved in dioxane (2 mL). The solution was purged with a stream of nitrogen for 15 min. [1,1′-bis(di-tert-butylphosphino)ferrocene]dichloropalladium(II) (4 mg, 0.006 mmol) was added and the resulting solution was heated at 100° C. for 3 h. The reaction mixture was cooled to room temperature and diluted with EtOAc (20 mL). The organic phase was washed with water (10 mL) and brine (10 mL), dried over magnesium sulfate, filtered, and concentrated. The crude residue was purified by column chromatography (0→6% MeOH/DCM to provide the desired product (51 mg, 0.07 mmol, 55% yield) as an off-white solid. LC/MS (ESI) m/z: [M+H]⁺ calcd for C₄₂H₄₃F₂N₆O₅ 749.3. found 749.3.

### Step 4: 2-((S)-4-(8-fluoro-2-(((2R,7aS)-2-fluorotetrahydro-1H-pyrrolizin-7a(5H)-yl)methoxy)-7-(3-(methoxymethoxy)naphthalen-1-yl)quinazolin-4-yl)piperazin-2-yl)acetonitrile

Benzyl (S)-2-(cyanomethyl)-4-(8-fluoro-2-(((2R,7aS)-2-fluorotetrahydro-1H-pyrrolizin-7a(5H)-yl)methoxy)-7-(3-(methoxymethoxy)naphthalen-1-yl)quinazolin-4-yl)piperazine-1-carboxylate (from Step 3, 50 mg, 0.07 mmol) and palladium on carbon (10 wt. %, 36 mg, 0.03 mmol) were suspended in ethanol (10 mL). Hydrogen gas was bubbled through the reaction mixture for 5 min. The reaction mixture was kept under a positive pressure of hydrogen (1 atm, balloon) for 2 h. The reaction mixture was filtered through diatomaceous earth (Celite™), taking care not to allow the filter cake to go dry, and the filtrate was concentrated. The crude residue was of sufficient purity to be used directly in the next step, without additional purification (quantitative yield assumed). LC/MS (ESI) m/z: [M+H]⁺ calcd for C₃₄H₃₇F₂N₆O₃ 615.3. found 615.2.

### Step 5: 2-((S)-4-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)piperazin-2-yl)acetonitrile

2-((S)-4-(8-fluoro-2-(((2R,7aS)-2-fluorotetrahydro-1H-pyrrolizin-7a(5H)-yl)methoxy)-7-(3-(methoxymethoxy)naphthalen-1-yl)quinazolin-4-yl)piperazin-2-yl)acetonitrile (from Step 4, 37 mg, 0.06 mmol) was suspended in HCl solution (4.0 M in dioxane, 0.5 mL, 2.0 mmol) and the reaction mixture was stirred at room temperature for 1 h. The mixture was directly concentrated and further dried on high vacuum to provide the desired product as the HCl salt (quantitative yield assumed). The crude material was of sufficient purity to be used directly in the next step, without additional purification. LC/MS (ESI) m/z: [M+H]⁺ calcd for C₃₂H₃₃F₂N₆O₂ 571.3. found 571.2.

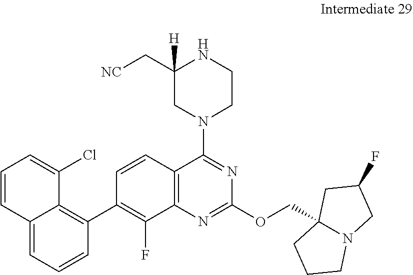 [SMILES: N#CC[C@H]1CN(c2nc(OC[C@@]34CCCN3C[C@H](F)C4)nc3c(F)c(-c4cccc5cccc(Cl)c45)ccc23)CCN1]

### 2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile

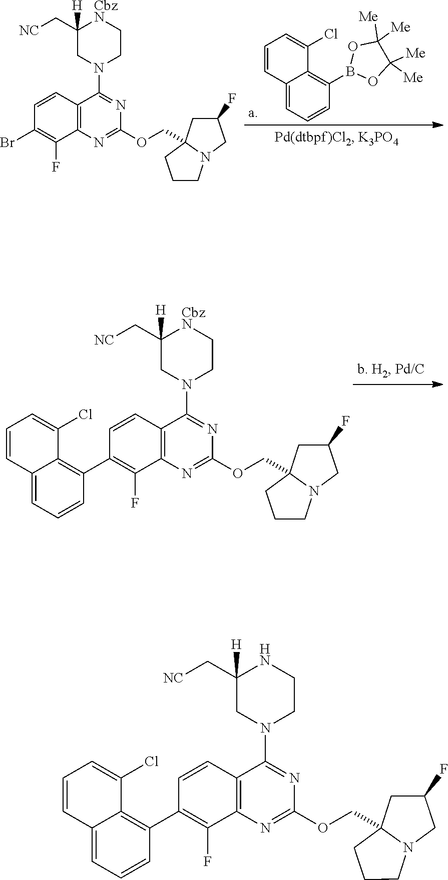

### Step 1: Benzyl (S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate

Benzyl (S)-4-(7-bromo-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate (140 mg, 0.22 mmol), 2-(8-chloronaphthalen-1-yl)-4,4,5,5-tetramethyl-1,3,2-dioxaborolane (63 mg, 0.22 mmol), and potassium phosphate tribasic solution (2.0 M, 0.33 mL, 0.66 mmol) were dissolved in dioxane (2 mL). The solution was purged with a stream of nitrogen for 15 min. [1,1′-bis(di-tert-butylphosphino)ferrocene]dichloropalladium(II) (7 mg, 0.01 mmol) was added and the resulting solution was heated at 100° C. for 3 h. The reaction mixture was cooled to room temperature and diluted with EtOAc (20 mL). The organic phase was washed with water (10 mL) and brine (10 mL), dried over magnesium sulfate, filtered, and concentrated. The crude residue was purified by column chromatography (0→6% MeOH/DCM to provide the desired product (78 mg, 0.11 mmol, 49% yield) as an off-white solid. LC/MS (ESI) m/z: [M+H]⁺ calcd for C₄₀H₃₈ClF₂N₆O₅ 723.3; found 723.2.

### Step 2: 2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile

Benzyl (S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate (Step 1, 58 mg, 0.08 mmol) and palladium on carbon (10 wt. %, 36 mg, 0.03 mmol) were suspended in ethanol (10 mL). Hydrogen gas was bubbled through the reaction mixture for 5 min. The reaction mixture was kept under a positive pressure of hydrogen (1 atm, balloon) for 2 h. The reaction mixture was filtered through diatomaceous earth (Celite™), taking care not to allow the filter cake to go dry, and the filtrate was concentrated. The crude residue was of sufficient purity to be used directly in the next step, without additional purification (quantitative yield assumed). LC/MS (ESI) m/z: [M+H]⁺ calcd for C₃₂H₃₃ClF₂N₆O 589.3. found 589.2.

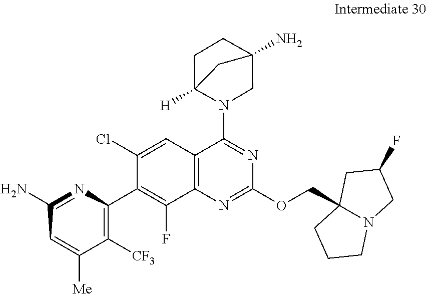

### (1S,4S)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-4-amine

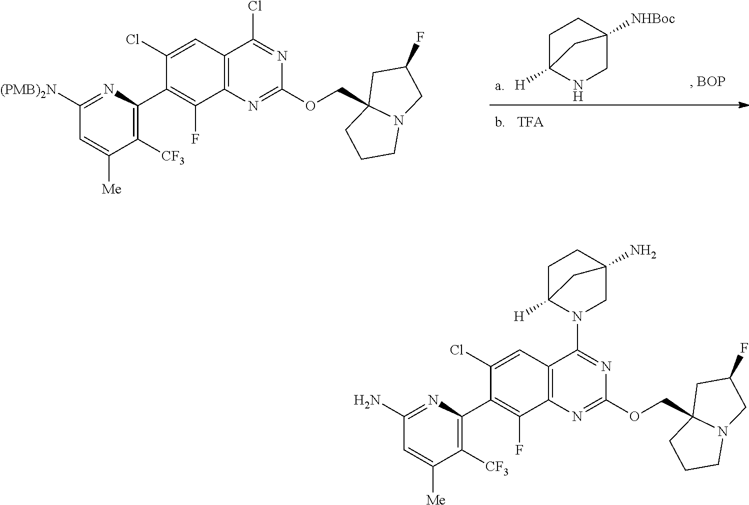

### Step 1: tert-butyl N-[(1S,4S)-2-[(7M)-2-{[(2R,7aR)-2-fluoro-hexahydro-1H-pyrrolizin-7a-yl]methoxy}-7-(6-{bis[(4-methoxyphenyl)methyl]amino}-4-methyl-3-(trifluoromethyl)pyridin-2-yl)-6-chloro-8-fluoroquinazolin-4-yl]-2-azabicyclo[2.2.1]heptan-4-yl]carbamate

7-(6-(Bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 50 mg, 0.065 mmol) and benzotriazol-1-yloxy)tris(dimethylamino)phosphonium hexafluorophosphate (57 mg, 0.13 mmol) were combined and dissolved in acetonitrile (5 mL). Triethylamine (18 μL, 0.13 mmol) was added followed by tert-butyl ((1S,4S)-2-azabicyclo[2.2.1]heptan-4-yl)carbamate (14 mg, 0.065 mmol). The reaction mixture was heated at 40° C. for 12 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue was of sufficient purity to be used directly in the next step, without additional purification (quantitative yield assumed). LC/MS (ESI) m/z: [M+H]⁺ calcd for C₅₀H₅₆ClF₅N₇O₅ 964.4, found 963.8.

### Step 2: (1S,4S)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-4-amine

Tert-butyl N-[(1S,4S)-2-[(7M)-2-{[(2R,7aR)-2-fluoro-hexahydro-1H-pyrrolizin-7a-yl]methoxy}-7-(6-{bis[(4-methoxyphenyl)methyl]amino}-4-methyl-3-(trifluoromethyl)pyridin-2-yl)-6-chloro-8-fluoroquinazolin-4-yl]-2-azabicyclo[2.2.1]heptan-4-yl]carbamate (from Step 1, 70 mg, 0.06 mmol) was dissolved in TFA (1.5 mL), and triethylsilane (10 μL, 0.06 mmol) and water (0.05 mL) were added. The reaction mixture was heated at 40° C. for 12 h. The crude mixture was directly purified by preparative HPLC to provide the desired product (15 mg, 0.02 mmol, 37% yield). LC/MS (ESI) m/z: [M+H]⁺ calcd for C₂₉H₃₂ClF₅N₇O 624.2. found 624.1; ¹H NMR (400 MHz, CD₃OD) δ 7.91-7.63 (m, 1H), 6.48 (s, 1H), 5.52-5.25 (m, 1H), 5.14-4.99 (m, 1H), 4.89 (br s, 1H), 4.45 (d, J=10.5 Hz, 1H), 4.31-4.21 (m, 1H), 3.95 (br d, J=8.8 Hz, 1H), 3.72-3.47 (m, 1H), 3.25-3.14 (m, 1H), 3.04-2.95 (m, 1H), 2.79-2.59 (m, 2H), 2.50 (s, 2H), 2.20 (dt, J=6.4, 12.9 Hz, 1H), 2.13-2.06 (m, 4H), 2.07-1.94 (m, 8H), 1.86-1.71 (m, 4H).

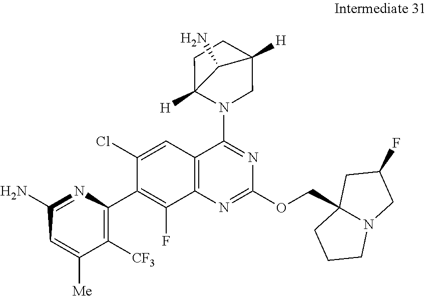

### (1R,4R,7R)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-7-amine

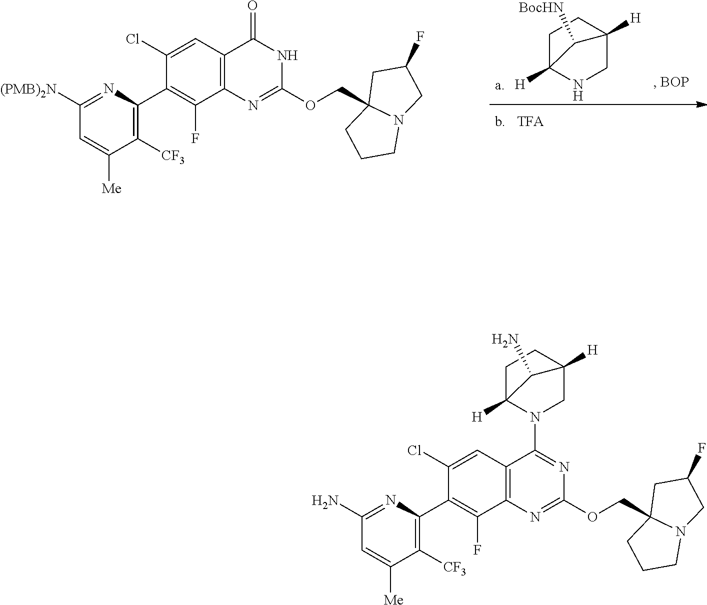

### Step 1: tert-butyl N-[(1R,4R,7R)-2-[(7M)-2-{[(2R,7aR)-2-fluoro-hexahydro-1H-pyrrolizin-7a-yl]methoxy}-7-(6-{bis[(4-methoxyphenyl)methyl]amino}-4-methyl-3-(trifluoromethyl)pyridin-2-yl)-6-chloro-8-fluoroquinazolin-4-yl]-2-azabicyclo[2.2.1]heptan-7-yl]carbamate

7-(6-(Bis(4-methoxybenzyl)amino)-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4(3H)-one (Intermediate 14B, 50 mg, 0.065 mmol) and benzotriazol-1-yloxy)tris(dimethylamino)phosphonium hexafluorophosphate (43 mg, 0.10 mmol) were combined and dissolved in acetonitrile (2 mL). Triethylamine (14 μL, 0.10 mmol) was added followed by tert-butyl ((7R)-2-azabicyclo[2.2.1]heptan-7-yl)carbamate (17 mg, 0.08 mmol). The reaction mixture was heated at 40° C. for 16 h. Water (10 mL) and EtOAc (20 mL) were added and the layers were separated. The aqueous layer was back extracted with EtOAc (2×10 mL). The combined organic phases were dried over sodium sulfate, filtered, and concentrated. The crude residue purified by column chromatography on neutral alumina (45% EtOAc/hexanes) to provide the desired product (52 mg, 0.05 mmol, 81% yield). LC/MS (ESI) m/z: [M+H]⁺ calcd for C₅₀H₅₆ClF₅N₇O₅ 964.4. found 964.5.

### Step 2: (1R,4R,7R)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-7-amine

tert-Butyl N-[(1R,4R,7R)-2-[(7M)-2-{[(2R,7aR)-2-fluoro-hexahydro-1H-pyrrolizin-7a-yl]methoxy}-7-(6-{bis[(4-methoxyphenyl)methyl]amino}-4-methyl-3-(trifluoromethyl)pyridin-2-yl)-6-chloro-8-fluoroquinazolin-4-yl]-2-azabicyclo[2.2.1]heptan-7-yl]carbamate (from Step 1, 52 mg, 0.05 mmol) was dissolved in TFA (1.5 mL), and triethylsilane (1 μL, 0.006 mmol) and water (0.05 mL) were added. The reaction mixture was heated at 40° C. for 12 h. The crude mixture was directly purified by preparative HPLC to provide the desired product as the TFA salt (26 mg, 0.035 mmol, 64% yield). LC/MS (ESI) m/z: [M+H]⁺ calcd for C₂₉H₃₂ClF₅N₇O 624.2. found 624.2; ¹H NMR (400 MHz, CD₃OD) δ 8.07 (s, 1H), 6.65 (s, 1H), 5.45-5.58 (m, 1H), 5.19 (s, 1H), 4.79-4.81 (m, 3H), 4.38-4.97 (m, 1H), 3.99-4.09 (m, 1H), 3.33-3.81 (m, 4H), 2.90-3.00 (m, 1H), 2.61-2.82 (m, 1H), 2.47-2.59 (m, 5H), 2.09 (m, 6H), 1.82-1.90 (m, 1H).

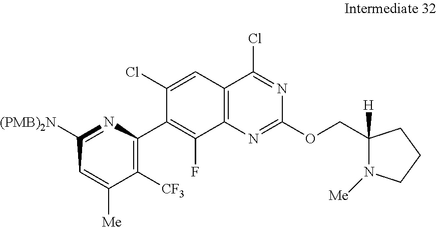

### (5M)-6-(4,6-dichloro-8-fluoro-2-{[(2S)-1-methylpyrrolidin-2-yl]methoxy}quinazolin-7-yl)-4-methyl-5-(trifluoromethyl)pyridin-2-amine

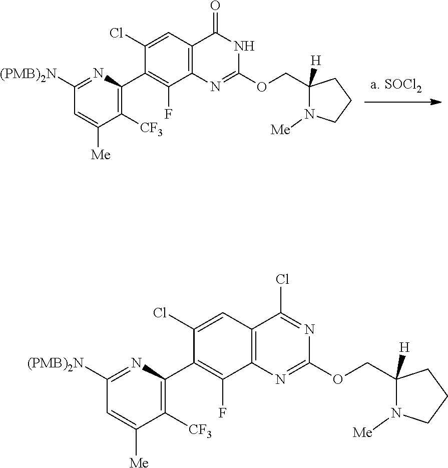

7-(6-(Bis(4-methoxybenzyl)amino)-4-methyl-3-(trifluoromethyl)pyridin-2-yl)-6-chloro-8-fluoro-2-(((S)-1-methylpyrrolidin-2-yl)methoxy)quinazolin-4(3H)-one (Intermediate 61, 240 mg, 0.33 mmol) was dissolved in DCM (1.6 mL) and thionyl chloride was added (240 μL, 3.3 mmol) followed by a drop of DMF (˜10 μL). The reaction mixture was stirred at room temperature for 6 h. The reaction mixture was directly concentrated and the crude product was azeotroped from toluene (3×2 mL) to provide the desired product, a brown solid, as the HCl salt (quantitative yield assumed). LC/MS (ESI) m/z: [M+H]⁺ calcd for C₃₇H₃₆C₁₂F₄N₅O₃ 744.2. found 744.1.

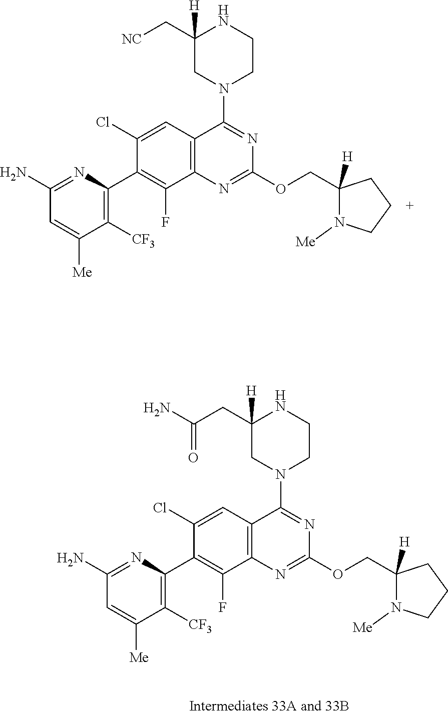

### 2-[(2S)-4-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]piperazin-2-yl]acetonitrile and 2-[(2S)-4-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]piperazin-2-yl]acetamide

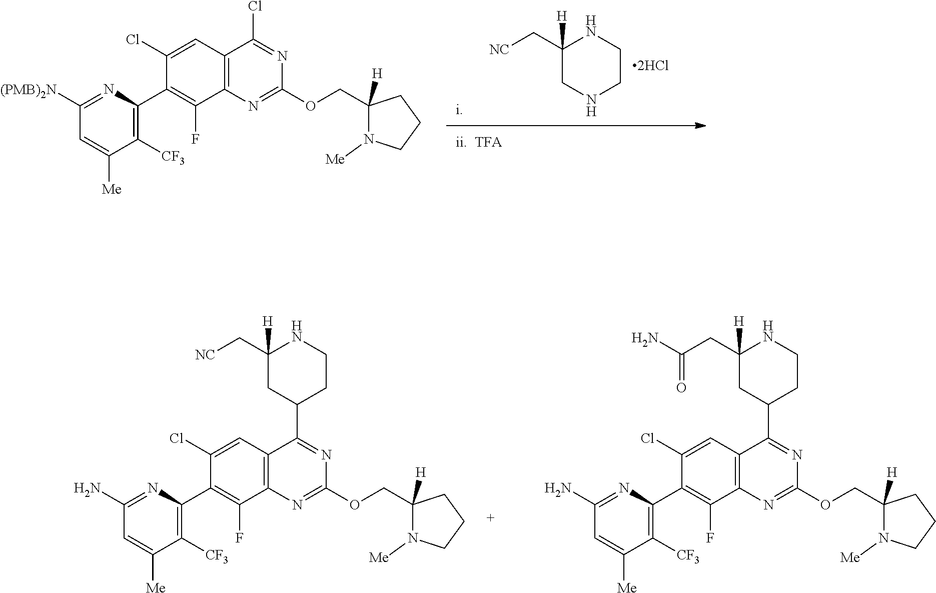

6-(4,6-Dichloro-8-fluoro-2-(((S)-1-methylpyrrolidin-2-yl)methoxy)quinazolin-7-yl)-4-methyl-5-(trifluoromethyl)pyridin-2-amine, HCl salt (Intermediate 32, 40 mg, 0.05 mmol) and (S)-2-(piperazin-2-yl)acetonitrile, 2HCl salt (15 mg, 0.08 mmol were combined and suspended in DCM (500 μL). DIPEA (45 μL, 0.26 mmol) was added and the reaction mixture was heated at 50° C. for 30 min. To the solution was then added triethylsilane (80 μL, 0.5 mmol), TFA (500 μL), and a drop of water (˜10 μL). The reaction mixture was heated at 50° C. for 16 h. The reaction mixture was directly purified by reverse phase HPLC (column: Xbridge C18, 19 mm×200 mm, 5 m particles; flow rate: 20 mL/min; column temperature: 25° C.; gradient: 87% 5:95 MeCN:H₂O with 10 mM AA/13% 95:5 MeCN:H₂O with 10 mM AA→100% 95:5 MeCN:H₂O with 10 mM AA; λ=220 nm) to provide the desired product (7.5 mg, 0.013 mmol, 25% yield) along with a nitrile hydrolysis product (8.7 mg, 0.014 mmol, 28% yield). LC/MS (ESI) m/z: [M+H]⁺ calcd for C₂₇H₃₀ClF₄N₈O 593.2. found 593.2 [desired]; and LC/MS (ESI) m/z: [M+H]⁺ calcd for C₂₇H₃₁ClF₄N₈O₂ 611.2. found 611.4 [hydrolysis]; ¹H NMR (600 MHz, DMSO-d₆) δ 7.83 (s, 1H), 6.85 (s, 2H), 6.49 (s, 1H), 4.39 (dd, J=10.8, 4.5 Hz, 1H), 4.24-4.09 (m, 3H), 3.27-3.21 (m, 2H), 3.10-3.00 (m, 3H), 2.97-2.86 (m, 2H), 2.75-2.70 (m, 2H), 2.60-2.54 (m, 1H), 2.37 (br s, 3H), 2.35 (s, 3H), 2.20-2.14 (m, 1H), 1.96-1.90 (m, 1H), 1.71-1.61 (m, 3H) [desired] and ¹H NMR (600 MHz, DMSO-d₆) δ 7.85 (s, 1H), 7.43-7.43 (m, 1H), 7.41 (br s, 1H), 6.89-6.83 (m, 3H), 6.49 (s, 1H), 4.39 (dd, J=10.6, 4.5 Hz, 1H), 4.24 (br d, J=12.7 Hz, 1H), 4.20-4.11 (m, 2H), 3.17 (br t, J=11.7 Hz, 1H), 3.09 (br d, J=5.8 Hz, 1H), 3.01 (br d, J=11.8 Hz, 1H), 2.98-2.93 (m, 2H), 2.91-2.85 (m, 1H), 2.60-2.54 (m, 1H), 2.37 (br s, 3H), 2.36 (br s, 3H), 2.23-2.14 (m, 3H), 1.97-1.91 (m, 1H), 1.71-1.60 (m, 3H) [hydrolysis].

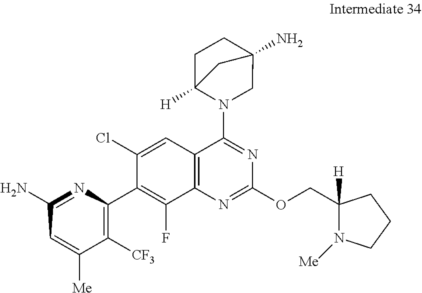

### (1S,4S)-2-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-2-azabicyclo[2.2.1]heptan-4-amine

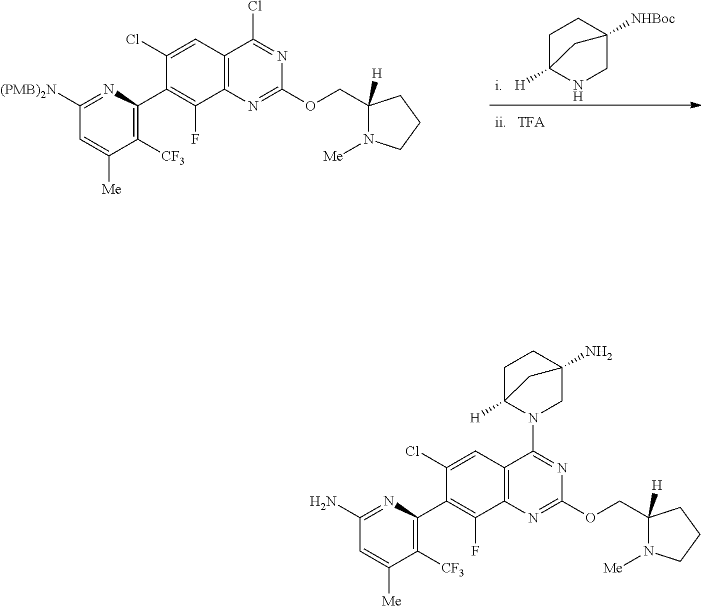

6-(4,6-Dichloro-8-fluoro-2-(((S)-1-methylpyrrolidin-2-yl)methoxy)quinazolin-7-yl)-4-methyl-5-(trifluoromethyl)pyridin-2-amine, HCl salt (Intermediate 32, 35 mg, 0.045 mmol) and tert-butyl ((1S,4S)-2-azabicyclo[2.2.1]heptan-4-yl)carbamate (Intermediate 37, 11 mg, 0.054 mmol were combined and suspended in DCM (500 μL). DIPEA (40 μL, 0.22 mmol) was added and the reaction mixture was heated at 50° C. for 30 min. To the solution was then added triethylsilane (29 μL, 0.18 mmol), TFA (500 μL), and a drop of water (˜10 μL). The reaction mixture was heated at 50° C. for 16 h. The reaction mixture was directly concentrated and the crude residue was azeotroped from toluene (3×1 mL) to provide the desired product as the TFA salt, which was used directly in the next step, without additional purification. LC/MS (ESI) m/z: [M+H]⁺ calcd for C₂₇H₃₁ClF₄N₇O 580.0. found 580.3.

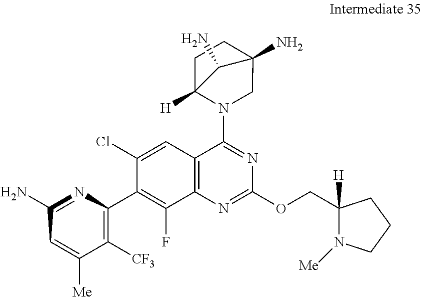

### (1R,4R,7R)-2-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-2-azabicyclo[2.2.1]heptan-7-amine

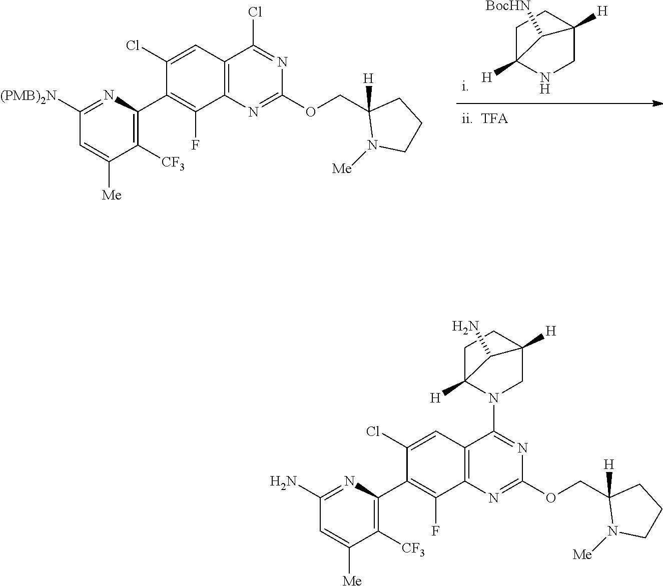

6-(4,6-Dichloro-8-fluoro-2-(((S)-1-methylpyrrolidin-2-yl)methoxy)quinazolin-7-yl)-4-methyl-5-(trifluoromethyl)pyridin-2-amine, HCl salt (Intermediate 32, 35 mg, 0.045 mmol) and tert-butyl ((1R,4R,7R)-2-azabicyclo[2.2.1]heptan-7-yl)carbamate (11 mg, 0.054 mmol were combined and suspended in DCM (500 μL). DIPEA (40 μL, 0.22 mmol) was added and the reaction mixture was heated at 50° C. for 30 min. To the solution was then added triethylsilane (29 μL, 0.18 mmol), TFA (500 μL), and a drop of water (˜10 μL). The reaction mixture was heated at 50° C. for 16 h. The reaction mixture was directly concentrated and the crude residue was azeotroped from toluene (3×1 mL) to provide the desired product as the TFA salt, which was used directly in the next step, without additional purification. LC/MS (ESI) m/z: [M+H]⁺ calcd for C₂₇H₃₁ClF₄N₇O 580.0. found 580.1.

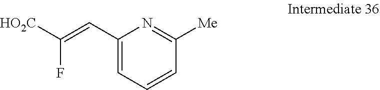 [SMILES: Cc1cccc(/C=C(\F)C(=O)O)n1]

### (Z)-2-fluoro-3-(6-methylpyridin-2-yl)acrylic acid

### Step 1: Ethyl (Z)-2-fluoro-3-(6-methylpyridin-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 6-methylpyridine-2-carbaldehyde (125 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by HPLC (column: Xbridge C18, m particles; gradient: 5:95 MeCN:H₂O with 0.05% TFA→95:5 MeCN:H₂O with 0.05% TFA; λ=220 nm) to provide the desired product (31 mg, 0.15 mmol, 14% yield); LC/MS (ESI) m/z: [M+H]⁺ calcd for C₁₁H₁₃FNO₂ 210.1. found 209.7; ¹H NMR (500 MHz, CDCl₃) δ 7.71 (d, J=7.8 Hz, 1H), 7.63 (t, J=7.8 Hz, 1H), 7.10 (d, J=7.8 Hz, 1H), 7.11 (d, J=35.2 Hz, 1H), 4.34 (q, J=7.2 Hz, 2H), 2.56 (s, 3H), 1.36 (t, J=7.2 Hz, 3H).

### Step 2: (Z)-2-fluoro-3-(6-methylpyridin-2-yl)acrylic acid

Ethyl (Z)-2-fluoro-3-(6-methylpyridin-4-yl)acrylate (31 mg, 0.15 mmol) was dissolved in MeOH (1.5 mL) and sodium hydroxide solution (1.0 M, 150 μL, 0.15 mmol) was added dropwise. The mixture was stirred for 2 h. The reaction mixture was concentrated, diluted with water (1 mL), and hydrochloric acid solution (1.0 M, 150 μL, 0.14 mmol) was added. This solution was lyophilized and the crude material was used without additional purification (quantitative yield assumed). LC/MS (ESI) m/z: [M+H]⁺ calcd for C₉H₉FNO₂ 182.1. found 182.1; ¹H NMR (500 MHz, DMSO-d₆) δ 7.80 (t, J=7.7 Hz, 1H), 7.65 (d, J=7.7 Hz, 1H), 7.29 (d, J=7.7 Hz, 1H), 6.94 (d, J=35.4 Hz, 1H), 2.50 (s, 3H).

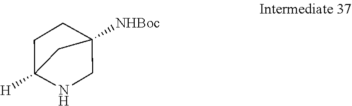 [SMILES: CC(C)(C)OC(=O)N[C@@]12CC[C@@H](C1)NC2]

### Tert-butyl ((1S,4S)-2-azabicyclo[2.2.1]heptan-4-yl)carbamate

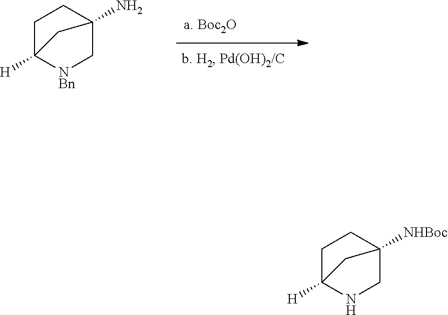 [SMILES: N[C@@]12CC[C@@H](C1)N(Cc1ccccc1)C2 ; CC(C)(C)OC(=O)N[C@@]12CC[C@@H](C1)NC2]

### Step 1: Tert-butyl ((1S,4S)-2-benzyl-2-azabicyclo[2.2.1]heptan-4-yl)carbamate

(1S,4S)-2-Benzyl-2-azabicyclo[2.2.1]heptan-4-amine (commercially available, or can also be prepared by following procedures described in U.S. Pat. No. 8,476,295 B2) (20.2 g, 100 mmol) was dissolved in water (150 mL). The solution was cooled to 0° C. and solid NaOH (˜24 g) was added portionwise until the pH of the solution was ˜14. A solution of di-tert-butyl dicarbonate (26.2 g, 120 mmol) in DCM (300 mL) was added and the resulting solution was stirred for 1 h. The layers were separated and the aqueous layer was further extracted with DCM (2×100 mL). The combined organic phases were dried over sodium sulfate, filtered, and concentrated. The crude residue was recrystallized from hot heptanes to provide the desired product (24.7 g, 82 mmol, 82% yield) as a white solid. ¹H NMR (400 MHz, CDCl₃) δ 7.49-7.15 (m, 5H), 4.94-4.67 (m, 1H), 3.87-3.59 (m, 2H), 3.26-3.17 (m, 1H), 3.10-2.85 (m, 1H), 2.64-2.30 (m, 1H), 2.16-19.2 (m, 2H), 1.88-1.58 (m, 4H), 1.43 (s, 9H).

### Step 2: Tert-butyl ((1S,4S)-2-azabicyclo[2.2.1]heptan-4-yl)carbamate

Tert-butyl ((1S,4S)-2-benzyl-2-azabicyclo[2.2.1]heptan-4-yl)carbamate (17 g, 56 mmol) was dissolved in MeOH (250 mL) and palladium hydroxide on carbon (20 wt. %, 1.7 g) was added as a solid. Hydrogen gas was bubbled through the solution for 5 min and the reaction mixture was kept under an atmosphere of hydrogen for 16 h. The reaction mixture was filtered through diatomaceous earth (Celite™) and the solution was concentrated to provide the desired product (11 g, 52 mmol, 92% yield) as a white solid. ¹H NMR (400 MHz, CDCl₃) δ 5.09 (br s, 1H), 3.92-3.58 (m, 2H), 1.95 (br s, 1H), 2.03-1.66 (m, 6H), 1.30 (s, 9H).

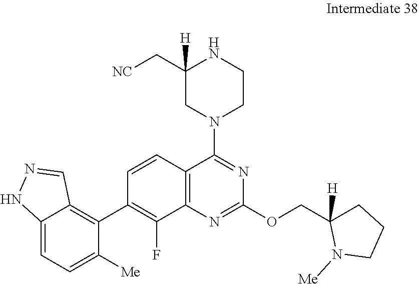 [SMILES: Cc1ccc2[nH]ncc2c1-c1ccc2c(N3CCN[C@@H](CC#N)C3)nc(OC[C@@H]3CCCN3C)nc2c1F]

### 2-((2S)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile

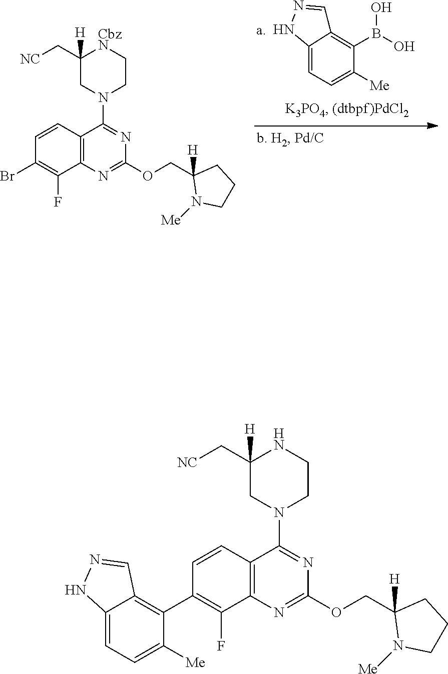

### Step 1: Preparation of benzyl (2S)-2-(cyanomethyl)-4-(8-fluoro-7-(5-methyl-1H-indazol-4-yl)-2-(((S)-1-methylpyrrolidin-2-yl)methoxy)quinazolin-4-yl)piperazine-1-carboxylate

Benzyl (S)-4-(7-bromo-8-fluoro-2-(((S)-1-methylpyrrolidin-2-yl)methoxy)quinazolin-4-yl)-2-(cyanomethyl)piperazine-1-carboxylate (200 mg, 0.335 mmol) and (5-methyl-1H-indazol-4-yl)boronic acid (59 mg, 0.335 mmol) were combined as solids and dissolved in dioxane (4 mL). Potassium phosphate solution (2.0 M, 0.5 mL, 1.0 mmol) was added and nitrogen was sparged through the solution for 15 min. [1,1′-bis(di-tert-butylphosphino)ferrocene]dichloropalladium(II), (dtbpf)PdCl₂, (11 mg, 0.017 mmol) was added as a solid, and the reaction mixture was heated at 100° C. for 2 h. The mixture was cooled, diluted with EtOAc (10 mL), and washed with water (5 mL) and brine (5 mL). The organic phase was dried over magnesium sulfate, filtered, and concentrated. The crude residue was purified by column chromatography (0→8% MeOH/DCM with 5% NH₄OH) to provide the desired product (159 mg, 0.245 mmol, 73% yield) as an off-white solid. LC/MS (ESI) m/z: [M+H]⁺ calcd for C₃₆H₃₈FN₈O₃ 649.3. found 649.3; ¹H NMR (500 MHz, CD₃OD) δ 7.94 (d, J=8.6 Hz, 1H), 7.60 (d, J=5.2 Hz, 1H), 7.55 (d, J=8.6 Hz, 1H), 7.44-7.29 (m, 7H), 5.27-5.16 (m, 2H), 4.83-4.74 (m, 3H), 4.56-4.51 (m, 1H), 4.49-4.38 (m, 3H), 4.20-4.12 (m, 1H), 3.78-3.69 (m, 1H), 3.66-3.51 (m, 2H), 3.12-2.94 (m, 3H), 2.85-2.77 (m, 1H), 2.53 (s, 1H), 2.42-2.34 (m, 1H), 2.30 (d, J=3.1 Hz, 3H), 2.17-2.06 (m, 1H), 1.86-1.73 (m, 3H).

### Step 2: Preparation of 2-((2S)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile

Benzyl (2S)-2-(cyanomethyl)-4-(8-fluoro-7-(5-methyl-1H-indazol-4-yl)-2-(((S)-1-methylpyrrolidin-2-yl)methoxy)quinazolin-4-yl)piperazine-1-carboxylate (150 mg, 0.23 mmol) was dissolved in EtOH (20 mL) and palladium on carbon (10 wt. %, 246 mg, 0.23 mmol) was added. Hydrogen gas was bubbled through the solution, and the reaction mixture was stirred under an atmosphere of hydrogen (balloon) for 1.5 h. The solution was filtered through a pad of Celite™ and concentrated to provide the desired product, which could be used directly in the next step, without additional purification (quantitative yield assumed). LC/MS (ESI) m/z: [M+H]⁺ calcd for C₂₈H₃₂FN₈O 515.3. found 515.2; ¹H NMR (500 MHz, CD₃OD) δ 7.90 (d, J=8.3 Hz, 1H), 7.61 (d, J=3.3 Hz, 1H), 7.54 (d, J=8.7 Hz, 1H), 7.41 (d, J=8.7 Hz, 1H), 7.31 (dd, J=8.3, 6.6 Hz, 1H), 4.58-4.45 (m, 3H), 4.42-4.34 (m, 1H), 3.51-3.42 (m, 1H), 3.36-3.32 (m, 1H), 3.24-2.99 (m, 5H), 2.85-2.79 (m, 1H), 2.72 (dd, J=6.3, 2.6 Hz, 2H), 2.54 (s, 3H), 2.41-2.34 (m, 1H), 2.30 (s, 3H), 2.17-2.08 (m, 1H), 1.89-1.76 (m, 3H).

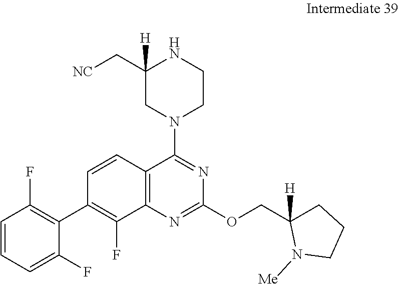 [SMILES: CN1CCC[C@H]1COc1nc(N2CCN[C@@H](CC#N)C2)c2ccc(-c3c(F)cccc3F)c(F)c2n1]

### 2-((2S)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile

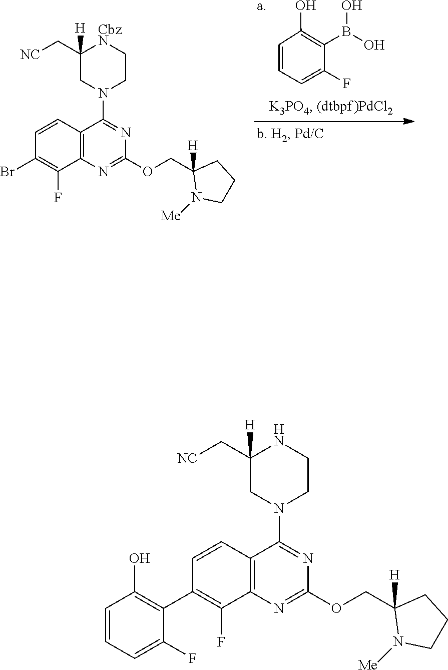

### Step 1: Preparation of benzyl (2S)-2-(cyanomethyl)-4-(8-fluoro-7-(2-fluoro-6-hydroxyphenyl)-2-(((S)-1-methylpyrrolidin-2-yl)methoxy)quinazolin-4-yl)piperazine-1-carboxylate

Benzyl (S)-4-(7-bromo-8-fluoro-2-(((S)-1-methylpyrrolidin-2-yl)methoxy)quinazolin-4-yl)-2-(cyanomethyl)piperazine-1-carboxylate (200 mg, 0.335 mmol) and (2-fluoro-6-hydroxyphenyl)boronic acid (52 mg, 0.335 mmol) were combined as solids and dissolved in dioxane (4 mL). Potassium phosphate solution (2.0 M, 0.5 mL, 1.0 mmol) was added and nitrogen was sparged through the solution for 15 min. [1,1′-bis(di-tert-butylphosphino)ferrocene]dichloropalladium(II), (dtbpf)PdCl₂, (11 mg, 0.017 mmol) was added as a solid, and the reaction mixture was heated at 100° C. for 2 h. The mixture was cooled, diluted with EtOAc (10 mL), and washed with water (5 mL) and brine (5 mL). The organic phase was dried over magnesium sulfate, filtered, and concentrated. The crude residue was purified by column chromatography (0→10% MeOH/DCM with 5% NH₄OH) to provide the desired product (160 mg, 0.25 mmol, 76% yield) as an off-white solid. LC/MS (ESI) m/z: [M+H]⁺ calcd for C₃₄H₃₅F₂N₆O₄ 629.3. found 629.4; ¹H NMR (500 MHz, CD₃OD) δ 7.82 (d, J=8.7 Hz, 1H), 7.44-7.29 (m, 6H), 7.28-7.23 (m, 1H), 6.77 (d, J=8.2 Hz, 1H), 6.70 (t, J=8.7 Hz, 1H), 5.25-5.16 (m, 2H), 4.82-4.77 (m, 2H), 4.56-4.49 (m, 1H), 4.49-4.43 (m, 1H), 4.43-4.35 (m, 2H), 4.19-4.09 (m, 1H), 3.73-3.61 (m, 1H), 3.61-3.42 (m, 2H), 3.13-2.95 (m, 2H), 2.87-2.79 (m, 1H), 2.54 (s, 3H), 2.40-2.32 (m, 1H), 2.18-2.07 (m, 1H), 1.88-1.73 (m, 3H).

### Step 2: Preparation of 2-((2S)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile

Benzyl (2S)-2-(cyanomethyl)-4-(8-fluoro-7-(2-fluoro-6-hydroxyphenyl)-2-(((S)-1-methylpyrrolidin-2-yl)methoxy)quinazolin-4-yl)piperazine-1-carboxylate (200 mg, 0.32 mmol) was dissolved in EtOH (20 mL), and palladium on carbon (10 wt. %, 340 mg, 0.32 mmol) was added. Hydrogen gas was bubbled through the solution, and the reaction mixture was stirred under an atmosphere of hydrogen (balloon) for 1.5 h. The solution was filtered through a pad of Celite™ and concentrated to provide the desired product, which could be used directly in the next step, without additional purification (quantitative yield assumed). LC/MS (ESI) m/z: [M+H]⁺ calcd for C₂₆H₂₉F₂N₆O₂ 495.2. found 495.2; ¹H NMR (500 MHz, CD₃OD) δ 7.77 (d, J=8.7 Hz, 1H), 7.31-7.22 (m, 2H), 6.76 (d, J=8.2 Hz, 1H), 6.73-6.66 (m, 1H), 4.56-4.50 (m, 1H), 4.50-4.40 (m, 2H), 4.36-4.30 (m, 1H), 3.46-3.38 (m, 1H), 3.30-3.26 (m, 1H), 3.19-3.07 (m, 3H), 3.06-2.98 (m, 1H), 2.85-2.77 (m, 1H), 2.73-2.67 (m, 2H), 2.54 (s, 3H), 2.43-2.32 (m, 1H), 2.18-2.05 (m, 1H), 1.90-1.72 (m, 3H).

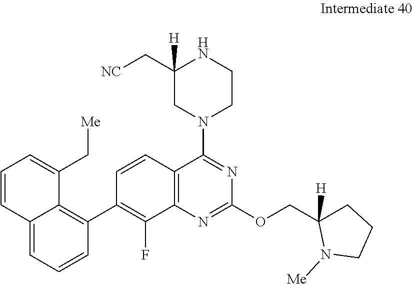 [SMILES: CCc1cccc2cccc(-c3ccc4c(N5CCN[C@@H](CC#N)C5)nc(OC[C@@H]5CCCN5C)nc4c3F)c12]

### 2-((S)-4-(7-(8-ethylnaphthalen-1-yl)-8-fluoro-2-(((S)-1-methylpyrrolidin-2-yl)methoxy)quinazolin-4-yl)piperazin-2-yl)acetonitrile

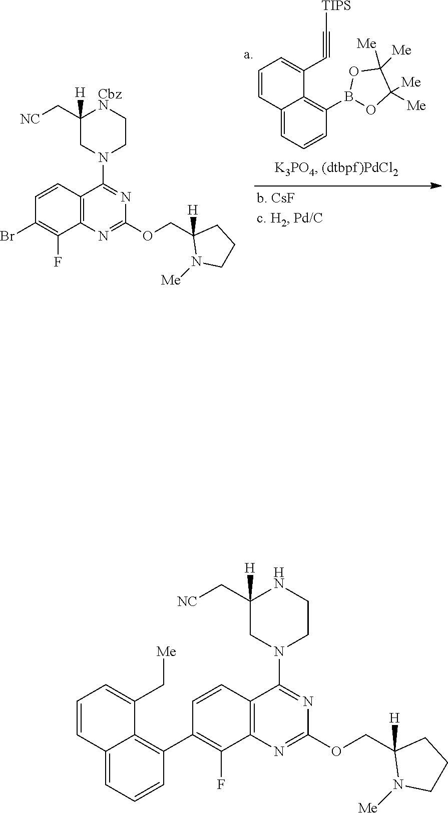

### Step 1: Preparation of benzyl (S)-2-(cyanomethyl)-4-(8-fluoro-2-(((S)-1-methylpyrrolidin-2-yl)methoxy)-7-(8-((triisopropylsilyl)ethynyl)naphthalen-1-yl)quinazolin-4-yl)piperazine-1-carboxylate

Benzyl (S)-4-(7-bromo-8-fluoro-2-(((S)-1-methylpyrrolidin-2-yl)methoxy)quinazolin-4-yl)-2-(cyanomethyl)piperazine-1-carboxylate (70 mg, 0.12 mmol) and (8-((triisopropylsilyl)ethynyl)naphthalen-1-yl)boronic acid (41 mg, 0.12 mmol) were combined as solids and dissolved in dioxane (4 mL). Potassium phosphate solution (2.0 M, 0.18 mL, 0.35 mmol) was added and nitrogen was sparged through the solution for 15 min. [1,1′-bis(di-tert-butylphosphino)ferrocene]dichloropalladium(II), (dtbpf)PdCl₂, (3.8 mg, 5.9 mol) was added as a solid, and the reaction mixture was heated at 100° C. for 2 h. The mixture was cooled, diluted with EtOAc (10 mL), and washed with water (5 mL) and brine (5 mL). The organic phase was dried over magnesium sulfate, filtered, and concentrated. The crude residue was purified by column chromatography (0→8% MeOH/DCM with 5% NH₄OH) to provide the desired product (60 mg, 0.07 mmol, 62% yield) as an off-white solid. LC/MS (ESI) m/z: [M+H]⁺ calcd for C₄₉H₅₈FN₆O₃Si 825.4. found 825.8; ¹H NMR (500 MHz, CD₃OD) δ 8.05-8.00 (m, 2H), 7.88-7.83 (m, 1H), 7.80-7.76 (m, 1H), 7.63-7.58 (m, 1H), 7.54-7.49 (m, 1H), 7.45-7.34 (m, 7H), 5.27-5.10 (m, 3H), 4.65-4.29 (m, 6H), 4.52-4.28 (m, 3H), 3.83-3.60 (m, 1H), 3.56-3.37 (m, 1H), 3.20-2.89 (m, 4H), 2.51-2.41 (m, 1H), 2.22 (s, 1H), 1.89-1.75 (m, 3H), 0.90-0.85 (m, 18H), 0.62-0.45 (m, 3H).

### Step 2: Preparation of benzyl (S)-2-(cyanomethyl)-4-(7-(8-ethynylnaphthalen-1-yl)-8-fluoro-2-(((S)-1-methylpyrrolidin-2-yl)methoxy)quinazolin-4-yl)piperazine-1-carboxylate

Benzyl (S)-2-(cyanomethyl)-4-(8-fluoro-2-(((S)-1-methylpyrrolidin-2-yl)methoxy)-7-(8-((triisopropylsilyl)ethynyl)naphthalen-1-yl)quinazolin-4-yl)piperazine-1-carboxylate (50 mg, 0.061 mmol) was dissolved in DMF (4 mL) and CsF (28 mg, 0.18 mmol) was added. The suspension was heated at 60° C. for 2 h. The reaction mixture was cooled to room temperature and was diluted with EtOAc (10 mL) and brine (5 mL). The layers were separated, and the organic phase was dried over magnesium sulfate, filtered, and concentrated. The crude residue was used directly in the next step, without additional purification. LC/MS (ESI) m/z: [M+H]⁺ calcd for C₄₀H₃₈FN₆O₃ 669.3. found 669.5.

### Step 3: Preparation of 2-((S)-4-(7-(8-ethylnaphthalen-1-yl)-8-fluoro-2-(((S)-1-methylpyrrolidin-2-yl)methoxy)quinazolin-4-yl)piperazin-2-yl)acetonitrile

Benzyl (S)-2-(cyanomethyl)-4-(7-(8-ethynylnaphthalen-1-yl)-8-fluoro-2-(((S)-1-methylpyrrolidin-2-yl)methoxy)quinazolin-4-yl)piperazine-1-carboxylate (50 mg, 0.075 mmol) was dissolved in EtOH (10 mL), and palladium on carbon (10 wt. %, 80 mg, 0.075 mmol) was added. Hydrogen gas was bubbled through the solution, and the reaction mixture was stirred under an atmosphere of hydrogen (balloon) for 1.5 h. The solution was filtered through a pad of Celite™ and concentrated to provide the desired product, which could be used directly in the next step, without additional purification (quantitative yield assumed). LC/MS (ESI) m/z: [M+H]⁺ calcd for C₃₂H₃₆FN₆O 539.3. found 539.2.

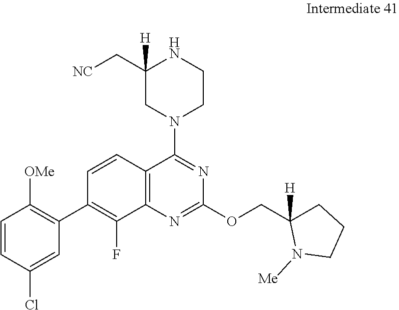 [SMILES: COc1ccc(Cl)cc1-c1ccc2c(N3CCN[C@@H](CC#N)C3)nc(OC[C@@H]3CCCN3C)nc2c1F]

### 2-((S)-4-(7-(5-chloro-2-methoxyphenyl)-8-fluoro-2-(((S)-1-methylpyrrolidin-2-yl)methoxy)quinazolin-4-yl)piperazin-2-yl)acetonitrile

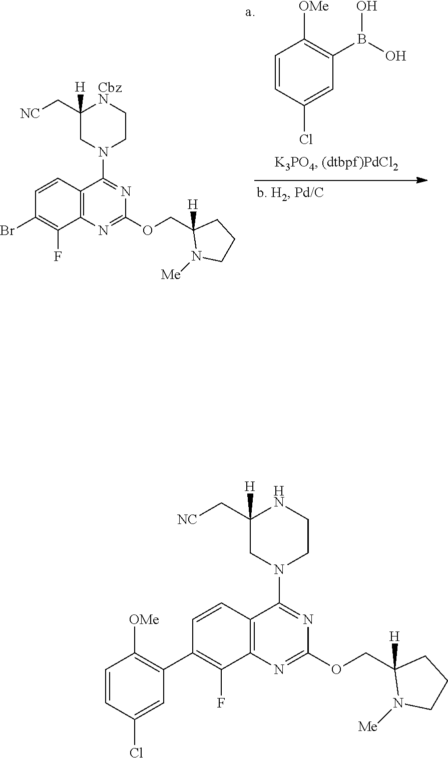 [SMILES: CN1CCC[C@H]1COc1nc(N2CCN(C(=O)OCc3ccccc3)[C@@H](CC#N)C2)c2ccc(Br)c(F)c2n1 ; "a." ; COc1ccc(Cl)cc1B(O)O ; COc1ccc(Cl)cc1-c1ccc2c(N3CCN[C@@H](CC#N)C3)nc(OC[C@@H]3CCCN3C)nc2c1F]

### Step 1: Preparation of benzyl (S)-4-(7-(5-chloro-2-methoxyphenyl)-8-fluoro-2-(((S)-1-methylpyrrolidin-2-yl)methoxy)quinazolin-4-yl)-2-(cyanomethyl)piperazine-1-carboxylate

Benzyl (S)-4-(7-bromo-8-fluoro-2-(((S)-1-methylpyrrolidin-2-yl)methoxy)quinazolin-4-yl)-2-(cyanomethyl)piperazine-1-carboxylate (70 mg, 0.117 mmol) and (5-chloro-2-methoxyphenyl)boronic acid (21.84 mg, 0.117 mmol) were combined as solids and dissolved in dioxane (4 mL). Potassium phosphate solution (2.0 M, 0.18 mL, 1.0 mmol) was added and nitrogen was sparged through the solution for 15 min. [1,1′-bis(di-tert-butylphosphino)ferrocene]dichloropalladium(II), (dtbpf)PdCl₂, (3.8 mg, 5.8 mol) was added as a solid, and the reaction mixture was heated at 100° C. for 2 h. The mixture was cooled, diluted with EtOAc (10 mL), and washed with water (5 mL) and brine (5 mL). The organic phase was dried over magnesium sulfate, filtered, and concentrated. The crude residue was purified by column chromatography (0→8% MeOH/DCM with 5% NH₄OH) to provide the desired product (55 mg, 0.08 mmol, 71% yield) as an off-white solid. LC/MS (ESI) m/z: [M+H]⁺ calcd for C₃₅H₃₇ClFN₆O₄ 659.3. found 659.4; ¹H NMR (500 MHz, CD₃OD) δ 7.82 (d, J=8.3 Hz, 1H), 7.43-7.28 (m, 8H), 7.13 (d, J=8.9 Hz, 1H), 5.26-5.15 (m, 2H), 4.80-4.76 (m, 2H), 4.62-4.49 (m, 2H), 4.46-4.35 (m, 2H), 4.17-4.11 (m, 1H), 3.80 (s, 3H), 3.73-3.68 (m, 1H), 3.60-3.48 (m, 1H), 3.18-3.13 (m, 1H), 3.06-2.96 (m, 2H), 2.92-2.86 (m, 1H), 2.57 (s, 3H), 2.48-2.42 (m, 1H), 2.16-2.12 (m, 1H), 1.89-1.78 (m, 3H).

### Step 2: Preparation of 2-((S)-4-(7-(5-chloro-2-methoxyphenyl)-8-fluoro-2-(((S)-1-methylpyrrolidin-2-yl)methoxy)quinazolin-4-yl)piperazin-2-yl)acetonitrile

Benzyl (S)-4-(7-(5-chloro-2-methoxyphenyl)-8-fluoro-2-(((S)-1-methylpyrrolidin-2-yl)methoxy)quinazolin-4-yl)-2-(cyanomethyl)piperazine-1-carboxylate (60 mg, 0.09 mmol) was dissolved in EtOH (15 mL), and palladium on carbon (10 wt. %, 97 mg, 0.09 mmol) was added. Hydrogen gas was bubbled through the solution, and the reaction mixture was stirred under an atmosphere of hydrogen (balloon) for 1.5 h. The solution was filtered through a pad of Celite™ and concentrated to provide the desired product, which could be used directly in the next step, without additional purification (quantitative yield assumed). LC/MS (ESI) m/z: [M+H]⁺ calcd for C₂₇H₃₁ClFN₆O₂ 525.2. found 525.4; ¹H NMR (500 MHz, CD₃OD) δ 7.77 (d, J=8.5 Hz, 1H), 7.42 (dd, J=8.9, 2.5 Hz, 1H), 7.31 (d, J=2.5 Hz, 1H), 7.26 (dd, J=8.5, 6.6 Hz, 1H), 7.12 (d, J=8.9 Hz, 1H), 4.54-4.41 (m, 4H), 4.37-4.29 (m, 1H), 3.80 (s, 3H), 3.46-3.40 (m, 1H), 3.19-3.10 (m, 3H), 3.05-2.99 (m, 1H), 2.84-2.78 (m, 2H), 2.72-2.68 (m, 2H), 2.54 (s, 3H), 2.40-2.35 (m, 1H), 2.16-2.09 (m, 1H), 1.86-1.77 (m, 3H).

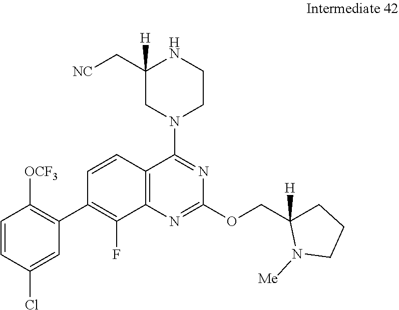 [SMILES: CN1CCC[C@H]1COc1nc(N2CCN[C@@H](CC#N)C2)c2ccc(-c3cc(Cl)ccc3OC(F)(F)F)c(F)c2n1]

### 2-((S)-4-(7-(5-chloro-2-(trifluoromethoxy)phenyl)-8-fluoro-2-(((S)-1-methylpyrrolidin-2-yl)methoxy)quinazolin-4-yl)piperazin-2-yl)acetonitrile

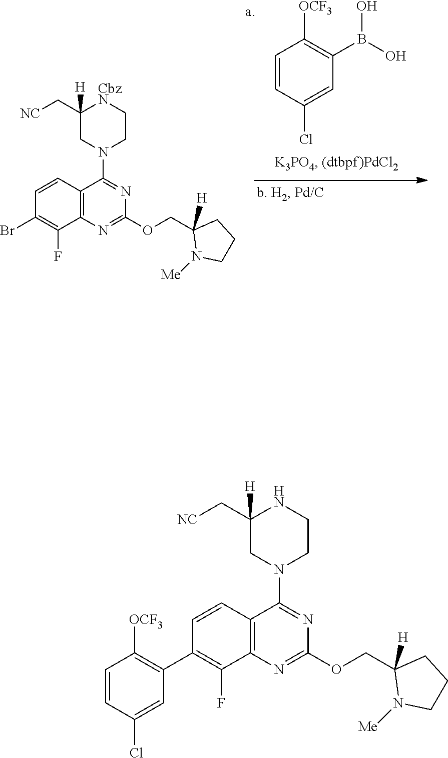 [SMILES: CN1CCC[C@H]1COc1nc(N2CCN(C(=O)OCc3ccccc3)[C@@H](CC#N)C2)c2ccc(Br)c(F)c2n1 ; "a." ; OB(O)c1cc(Cl)ccc1OC(F)(F)F ; CN1CCC[C@H]1COc1nc(N2CCN[C@@H](CC#N)C2)c2ccc(-c3cc(Cl)ccc3OC(F)(F)F)c(F)c2n1]

### Step 1: Preparation of benzyl (S)-4-(7-(5-chloro-2-(trifluoromethoxy)phenyl)-8-fluoro-2-(((S)-1-methylpyrrolidin-2-yl)methoxy)quinazolin-4-yl)-2-(cyanomethyl)piperazine-1-carboxylate

Benzyl (S)-4-(7-bromo-8-fluoro-2-(((S)-1-methylpyrrolidin-2-yl)methoxy)quinazolin-4-yl)-2-(cyanomethyl)piperazine-1-carboxylate (75 mg, 0.126 mmol) and (5-chloro-2-(trifluoromethoxy)phenyl)boronic acid (30 mg, 0.126 mmol) were combined as solids and dissolved in dioxane (4 mL). Potassium phosphate solution (2.0 M, 0.19 mL, 1.0 mmol) was added and nitrogen was sparged through the solution for 15 min. [1,1′-bis(di-tert-butylphosphino)ferrocene]dichloropalladium(II), (dtbpf)PdCl₂, (4.1 mg, 6.3 μmol) was added as a solid, and the reaction mixture was heated at 100° C. for 2 h. The mixture was cooled, diluted with EtOAc (10 mL), and washed with water (5 mL) and brine (5 mL). The organic phase was dried over magnesium sulfate, filtered, and concentrated. The crude residue was purified by column chromatography (0→8% MeOH/DCM with 5% NH₄OH) to provide the desired product (67 mg, 0.09 mmol, 75% yield) as an off-white solid. LC/MS (ESI) m/z: [M+H]⁺ calcd for C₃₅H₃₄ClF₄N₆O₄ 713.2. found 713.5; ¹H NMR (500 MHz, CD₃OD) δ 7.91 (d, J=8.7 Hz, 1H), 7.61-7.57 (m, 1H), 7.51-7.48 (m, 1H), 7.42-7.29 (m, 5H), 5.25-5.13 (m, 2H), 4.78-4.72 (m, 1H), 4.57 (br s, 3H), 4.54-4.46 (m, 2H), 4.43-4.31 (m, 2H), 4.19-4.10 (m, 1H), 3.74-3.66 (m, 1H), 3.60-3.53 (m, 1H), 3.20-3.12 (m, 1H), 3.09-2.93 (m, 2H), 2.91-2.81 (m, 1H), 2.56 (s, 3H), 2.48-2.37 (m, 1H), 2.20-2.06 (m, 1H), 1.93-1.75 (m, 3H).

### Step 2: Preparation of 2-((S)-4-(7-(5-chloro-2-(trifluoromethoxy)phenyl)-8-fluoro-2-(((S)-1-methylpyrrolidin-2-yl)methoxy)quinazolin-4-yl)piperazin-2-yl)acetonitrile

Benzyl (S)-4-(7-(5-chloro-2-(trifluoromethoxy)phenyl)-8-fluoro-2-(((S)-1-methylpyrrolidin-2-yl)methoxy)quinazolin-4-yl)-2-(cyanomethyl)piperazine-1-carboxylate (65 mg, 0.09 mmol) was dissolved in EtOH (15 mL), and palladium on carbon (10 wt. %, 97 mg, 0.09 mmol) was added. Hydrogen gas was bubbled through the solution, and the reaction mixture was stirred under an atmosphere of hydrogen (balloon) for 1.5 h. The solution was filtered through a pad of Celite™ and concentrated to provide the desired product, which could be used directly in the next step, without additional purification (quantitative yield assumed). LC/MS (ESI) m/z: [M+H]⁺ calcd for C₂₇H₂₈ClF₄N₆O₂ 579.2. found 579.2.

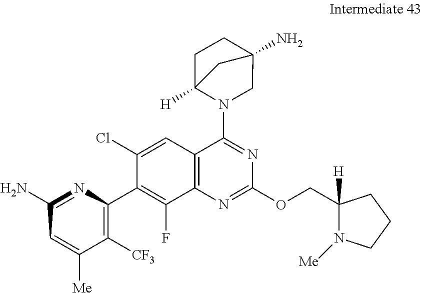

### (1S,4S)-2-((R)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-2-azabicyclo[2.2.1]heptan-4-amine

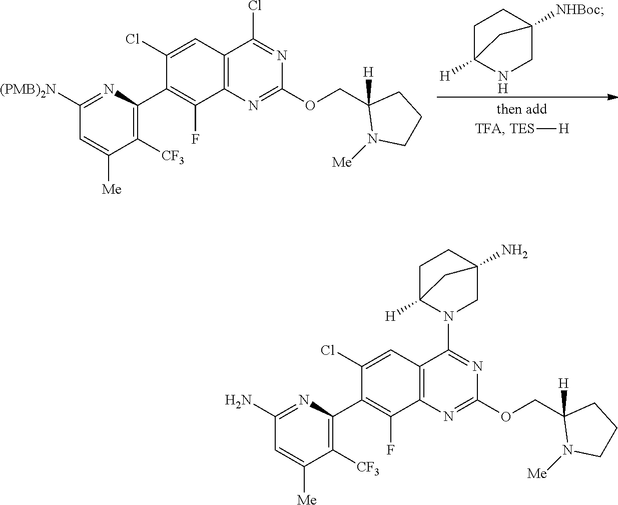

6-((R)-4,6-dichloro-8-fluoro-2-(((S)-1-methylpyrrolidin-2-yl)methoxy)quinazolin-7-yl)-N,N-bis(4-methoxybenzyl)-4-methyl-5-(trifluoromethyl)pyridin-2-amine, HCl (35 mg, 0.045 mmol) and tert-butyl ((1S,4S)-2-azabicyclo[2.2.1]heptan-4-yl)carbamate (11.42 mg, 0.054 mmol) were combined and suspended in DCM (0.5 mL). DIPEA (39 μl, 0.22 mmol) was added and the reaction mixture was heated at 40° C. for 15 min. Triethylsilane (29 μl, 0.18 mmol) and TFA (0.5 mL) were added to this solution, and the resulting mixture was kept at 40° C. for an additional 16 h. The reaction mixture was directly concentrated, and the crude residue was azeotroped from toluene (3×1 mL) to provide the desired product as a crude oil, which was used directly in the next step, without additional purification. LC/MS (ESI) m/z: [M+H]⁺ calcd for C₂₇H₃₁ClF₄N₇O 580.2. found 580.3.

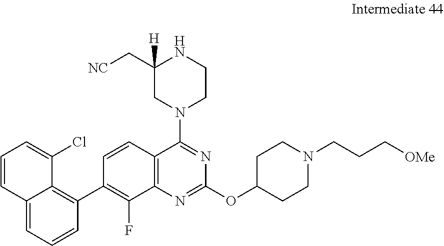 [SMILES: COCCCN1CCC(Oc2nc(N3CCN[C@@H](CC#N)C3)c3ccc(-c4cccc5cccc(Cl)c45)c(F)c3n2)CC1]

### (S)-2-(4-(7-(8-chloronaphthalen-1-yl)-8-fluoro-2-((1-(3-methoxypropyl)piperidin-4-yl)oxy)quinazolin-4-yl)piperazin-2-yl)acetonitrile

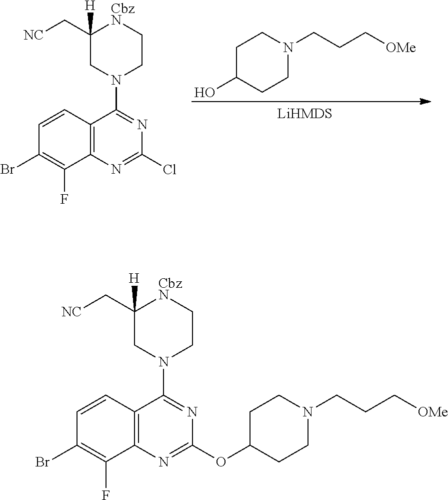 [SMILES: N#CC[C@H]1CN(c2nc(Cl)nc3c(F)c(Br)ccc23)CCN1C(=O)OCc1ccccc1 ; COCCCN1CCC(O)CC1 ; COCCCN1CCC(Oc2nc(N3CCN(C(=O)OCc4ccccc4)[C@@H](CC#N)C3)c3ccc(Br)c(F)c3n2)CC1]

### Step 1: Preparation of benzyl (S)-4-(7-bromo-8-fluoro-2-((1-(3-methoxypropyl)piperidin-4-yl)oxy)quinazolin-4-yl)-2-(cyanomethyl)piperazine-1-carboxylate

(S)-4-(7-Bromo-2-chloro-8-fluoroquinazolin-4-yl)-2-(cyanomethyl)piperazine-1-carboxylate (120 mg, 0.23 mmol) and 1-(3-methoxypropyl)piperidin-4-ol (40 mg, 0.23 mmol) were dissolved in THE (5 mL) and LiHMDS solution (1.0 M in THF, 0.5 mL, 0.5 mmol) was added dropwise. The resulting solution was stirred at room temperature for 24 h. The reaction mixture was quenched with water (5 mL) and EtOAc (10 mL). The layers were separated and the organic phase was further washed with brine (5 mL), dried over magnesium sulfate, filtered, and concentrated. The crude residue was purified by column chromatography (0→8% MeOH/DCM with 5% NH₄OH) to provide the desired product (81 mg, 0.12 mmol, 53% yield) as a light yellow solid. LC/MS (ESI) m/z: [M+H]⁺ calcd for C₃₁H₃₆BrFN₆O₄ 655.2. found 655.4; ¹H NMR (500 MHz, CD₃OD) δ 7.73 (d, J=9.3 Hz, 1H), 7.49 (dd, J=9.3, 6.3 Hz, 1H), 7.44-7.30 (m, 5H), 5.27-5.08 (m, 3H), 4.68-4.55 (m, 2H), 4.33 (br d, J=4.5 Hz, 2H), 3.68 (s, 2H), 3.62-3.50 (m, 2H), 3.50-3.43 (m, 3H), 3.30 (br s, 1H), 3.10-2.94 (m, 2H), 2.94-2.74 (m, 2H), 2.57-2.43 (m, 2H), 2.29-2.08 (m, 2H), 1.99-1.88 (m, 2H), 1.87-1.73 (m, 2H), 1.73-1.60 (m, 2H).

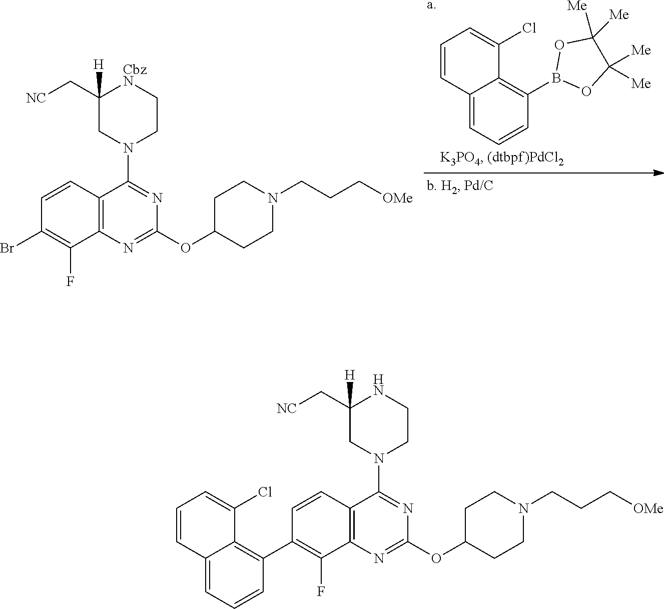

### Step 2: Preparation of benzyl (S)-4-(7-(8-chloronaphthalen-1-yl)-8-fluoro-2-((1-(3-methoxypropyl)piperidin-4-yl)oxy)quinazolin-4-yl)-2-(cyanomethyl)piperazine-1-carboxylate

Benzyl (S)-4-(7-bromo-8-fluoro-2-((1-(3-methoxypropyl)piperidin-4-yl)oxy)quinazolin-4-yl)-2-(cyanomethyl)piperazine-1-carboxylate (80 mg, 0.12 mmol) and 2-(8-chloronaphthalen-1-yl)-4,4,5,5-tetramethyl-1,3,2-dioxaborolane (35 mg, 0.12 mmol) were combined as solids and dissolved in dioxane (2 mL). Potassium phosphate solution (2.0 M, 0.18 mL, 1.0 mmol) was added and nitrogen was sparged through the solution for 15 min. [1,1′-bis(di-tert-butylphosphino)ferrocene]dichloropalladium(II), (dtbpf)PdCl₂, (4.0 mg, 6.1 mol) was added as a solid, and the reaction mixture was heated at 100° C. for 12 h. The mixture was cooled, diluted with EtOAc (10 mL), and washed with water (5 mL) and brine (5 mL). The organic phase was dried over magnesium sulfate, filtered, and concentrated. The crude residue was purified by column chromatography (0→8% MeOH/DCM with 5% NH₄OH) to provide the desired product (30 mg, 0.04 mmol, % yield) as an off-white solid. LC/MS (ESI) m/z: [M+H]⁻ calcd for C₄₁H₃₃ClFN₆O₄ 737.3. found 737.2.

### Step 3: Preparation of (S)-2-(4-(7-(8-chloronaphthalen-1-yl)-8-fluoro-2-((1-(3-methoxypropyl)piperidin-4-yl)oxy)quinazolin-4-yl)piperazin-2-yl)acetonitrile

Benzyl (S)-4-(7-(8-chloronaphthalen-1-yl)-8-fluoro-2-((1-(3-methoxypropyl)piperidin-4-yl)oxy)quinazolin-4-yl)-2-(cyanomethyl)piperazine-1-carboxylate (30 mg, 0.04 mmol) was dissolved in EtOH (10 mL), and palladium on carbon (10 wt. %, 43 mg, 0.04 mmol) was added. Hydrogen gas was bubbled through the solution, and the reaction mixture was stirred under an atmosphere of hydrogen (balloon) for 1.5 h. The solution was filtered through a pad of Celite™ and concentrated to provide the desired product, which could be used directly in the next step, without additional purification (quantitative yield assumed). LC/MS (ESI) m/z: [M+H]⁺ calcd for C₃₃H₃₇ClFN₆O₂ 603.2. found 603.5; ¹H NMR (500 MHz, CD₃OD) δ 7.99 (d, J=7.7 Hz, 1H), 7.85 (br d, J=8.9 Hz, 2H) 7.77 (d, J=8.0 Hz, 2H), 7.55-7.40 (m, 1H), 7.37-7.24 (m, 2H), 5.29-5.19 (m, 1H), 4.63 (br s, 1H), 4.29 (br dd, J=15.3, 2.6 Hz, 2H), 4.15 (t, J=6.8 Hz, 1H), 3.52-3.38 (m, 3H), 3.36-3.34 (m, 2H), 3.24-3.08 (m, 2H), 3.07-2.96 (m, 2H), 2.93-2.81 (m, 2H), 2.80-2.65 (m, 2H), 2.55-2.41 (m, 1H), 2.39-2.28 (m, 2H), 2.23-2.11 (m, 1H), 1.99-1.88 (m, 2H), 1.86-1.65 (m, 2H), 1.42-1.23 (m, 2H).

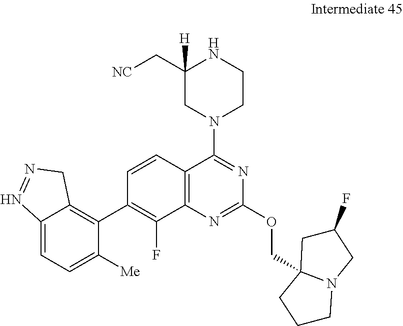 [SMILES: Cc1ccc2c(c1-c1ccc3c(N4CCN[C@@H](CC#N)C4)nc(OC[C@@]45CCCN4C[C@H](F)C5)nc3c1F)CN=N2]

### 2-((2S)-4-(8-fluoro-2-(((2R,7aS)-2-fluorotetrahydro-1H-pyrrolizin-7a(5H)-yl)methoxy)-7-(5-methyl-1H-indazol-4-yl)quinazolin-4-yl)piperazin-2-yl)acetonitrile

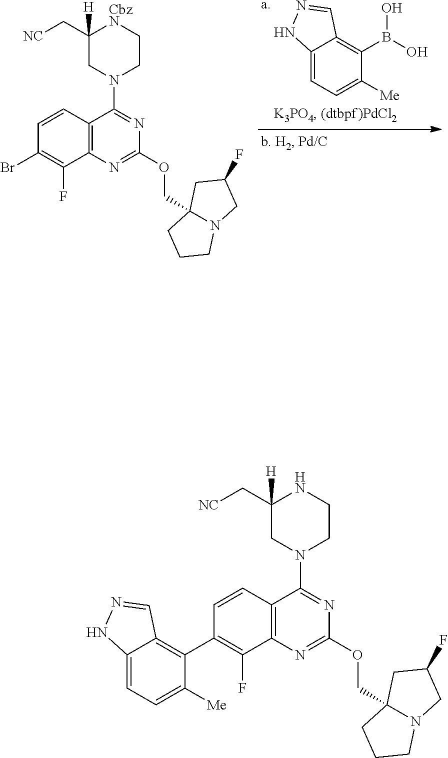

### Step 1: Preparation of benzyl (2S)-2-(cyanomethyl)-4-(8-fluoro-2-(((2R,7aS)-2-fluorotetrahydro-1H-pyrrolizin-7a(5H)-yl)methoxy)-7-(5-methyl-1H-indazol-4-yl)quinazolin-4-yl)piperazine-1-carboxylate

Benzyl (S)-4-(7-bromo-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate (70 mg, 0.11 mmol) and (5-methyl-1H-indazol-4-yl)boronic acid (19 mg, 0.11 mmol) were combined as solids and dissolved in dioxane (2 mL). Potassium phosphate solution (2.0 M, 0.16 mL, 0.33 mmol) was added and nitrogen was sparged through the solution for 15 min. [1,1′-bis(di-tert-butylphosphino)ferrocene]dichloropalladium(II), (dtbpf)PdCl₂, (4.0 mg, 6.1 mol) was added as a solid, and the reaction mixture was heated at 100° C. for 12 h. The mixture was cooled, diluted with EtOAc (10 mL), and washed with water (5 mL) and brine (5 mL). The organic phase was dried over magnesium sulfate, filtered, and concentrated. The crude residue was purified by column chromatography (0→8% MeOH/DCM with 5% NH₄OH) to provide the desired product (39 mg, 0.06 mmol, 52% yield) as an off-white solid. LC/MS (ESI) m/z: [M+H]⁺ calcd for C₃₈H₃₉F₂N₈O₃ 693.3. found 693.5; ¹H NMR (500 MHz, CD₃OD) δ 7.94 (d, J=8.2 Hz, 1H), 7.61-7.58 (m, 1H), 7.55 (d, J=8.8 Hz, 1H), 7.42-7.32 (m, 7H), 5.31 (d, J=56.4 Hz, 1H), 5.22-5.17 (m, 2H), 4.60-4.53 (m, 2H), 4.50-4.41 (m, 2H), 4.33-4.23 (m, 2H), 4.18-4.12 (m, 2H), 3.78-3.69 (m, 1H), 3.63-3.50 (m, 2H), 3.47-3.41 (m, 1H), 3.21-3.14 (m, 2H), 3.06 (s, 4H), 2.27-2.12 (m, 3H), 2.03-1.95 (m, 2H), 1.94-1.87 (m, 1H)

### Step 2: Preparation of 2-((2S)-4-(8-fluoro-2-(((2R,7aS)-2-fluorotetrahydro-1H-pyrrolizin-7a(5H)-yl)methoxy)-7-(5-methyl-1H-indazol-4-yl)quinazolin-4-yl)piperazin-2-yl)acetonitrile

Benzyl (2S)-2-(cyanomethyl)-4-(8-fluoro-2-(((2R,7aS)-2-fluorotetrahydro-1H-pyrrolizin-7a(5H)-yl)methoxy)-7-(5-methyl-1H-indazol-4-yl)quinazolin-4-yl)piperazine-1-carboxylate (39 mg, 0.056 mmol) was dissolved in EtOH (10 mL), and palladium on carbon (10 wt. %, 59 mg, 0.056 mmol) was added. Hydrogen gas was bubbled through the solution, and the reaction mixture was stirred under an atmosphere of hydrogen (balloon) for 1.5 h. The solution was filtered through a pad of Celite™ and concentrated to provide the desired product, which could be used directly in the next step, without additional purification (quantitative yield assumed). LC/MS (ESI) m/z: [M+H]⁺ calcd for C₃₀H₃₃F₂N₈O 559.3. found 559.4; ¹H NMR (500 MHz, CD₃OD) δ 7.90 (d, J=8.9 Hz, 1H), 7.60 (d, J=3.9 Hz, 1H), 7.55 (d, J=8.8 Hz, 1H), 7.41 (d, J=8.5 Hz, 1H), 7.31 (ddd, J=8.6, 6.5, 1.9 Hz, 1H), 5.31 (d, J=55.8 Hz, 1H), 4.59-4.51 (m, 1H), 4.41-4.35 (m, 1H), 4.35-4.31 (m, 1H), 4.28-4.24 (m, 1H), 4.13 (t, J=6.7 Hz, 1H), 3.53-3.43 (m, 2H), 3.27-3.10 (m, 8H), 3.06-2.98 (m, 3H), 2.42-2.32 (m, 1H), 2.23-2.13 (m, 2H), 2.01-1.95 (m, 2H), 1.94-1.85 (m, 1H).

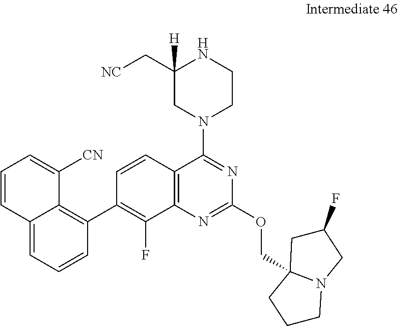 [SMILES: N#CC[C@H]1CN(c2nc(OC[C@@]34CCCN3C[C@H](F)C4)nc3c(F)c(-c4cccc5cccc(C#N)c45)ccc23)CCN1]

### 8-(4-((S)-3-(cyanomethyl)piperazin-1-yl)-8-fluoro-2-(((2R,7aS)-2-fluorotetrahydro-1H-pyrrolizin-7a(5H)-yl)methoxy)quinazolin-7-yl)-1-naphthonitrile

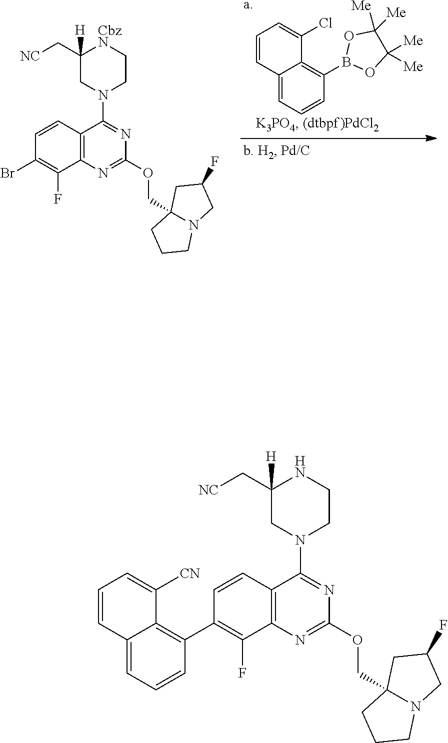

### Step 1: Preparation of benzyl (S)-2-(cyanomethyl)-4-(7-(8-cyanonaphthalen-1-yl)-8-fluoro-2-(((2R,7aS)-2-fluorotetrahydro-1H-pyrrolizin-7a(5H)-yl)methoxy)quinazolin-4-yl)piperazine-1-carboxylate

Benzyl (S)-2-(cyanomethyl)-4-(7-(8-cyanonaphthalen-1-yl)-8-fluoro-2-(((2R,7aS)-2-fluorotetrahydro-1H-pyrrolizin-7a(5H)-yl)methoxy)quinazolin-4-yl)piperazine-1-carboxylate (70 mg, 0.11 mmol) and 8-(4,4,5,5-tetramethyl-1,3,2-dioxaborolan-2-yl)-1-naphthonitrile (31 mg, 0.11 mmol were combined as solids and dissolved in dioxane (2 mL). Potassium phosphate solution (2.0 M, 0.16 mL, 0.33 mmol) was added and nitrogen was sparged through the solution for 15 min. [1,1′-bis(di-tert-butylphosphino)ferrocene]dichloropalladium(II), (dtbpf)PdCl₂, (3.6 mg, 5.5 mol) was added as a solid, and the reaction mixture was heated at 100° C. for 2 h. The mixture was cooled, diluted with EtOAc (10 mL), and washed with water (5 mL) and brine (5 mL). The organic phase was dried over magnesium sulfate, filtered, and concentrated. The crude residue was purified by column chromatography (0→8% MeOH/DCM with 5% NH₄OH) to provide the desired product (35 mg, 0.05 mmol, 45% yield) as an off-white solid. LC/MS (ESI) m/z: [M+H]⁺ calcd for C₄₁H₃₈F₂N₇O₃ 714.3. found 714.3.

### Step 2: Preparation of 8-(4-((S)-3-(cyanomethyl)piperazin-1-yl)-8-fluoro-2-(((2R,7aS)-2-fluorotetrahydro-1H-pyrrolizin-7a(5H)-yl)methoxy)quinazolin-7-yl)-1-naphthonitrile

Benzyl (S)-2-(cyanomethyl)-4-(7-(8-cyanonaphthalen-1-yl)-8-fluoro-2-(((2R,7aS)-2-fluorotetrahydro-1H-pyrrolizin-7a(5H)-yl)methoxy)quinazolin-4-yl)piperazine-1-carboxylate (30 mg, 0.04 mmol) was dissolved in EtOH (10 mL), and palladium on carbon (10 wt. %, 45 mg, 0.04 mmol) was added. Hydrogen gas was bubbled through the solution, and the reaction mixture was stirred under an atmosphere of hydrogen (balloon) for 1.5 h. The solution was filtered through a pad of Celite™ and concentrated to provide the desired product, which could be used directly in the next step, without additional purification (quantitative yield assumed). LC/MS (ESI) m/z: [M+H]⁺ calcd for C₃₃H₃₂F₂N₇O 559.3. found 559.4.

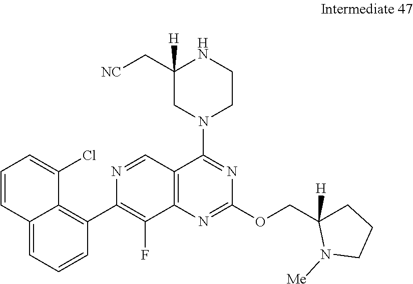 [SMILES: CN1CCC[C@H]1COc1nc(N2CCN[C@@H](CC#N)C2)c2cnc(-c3cccc4cccc(Cl)c34)c(F)c2n1]

### 8-(4-((S)-3-(cyanomethyl)piperazin-1-yl)-8-fluoro-2-(((S)-1-methylpyrrolidin-2-yl)methoxy)quinazolin-7-yl)-1-naphthonitrile

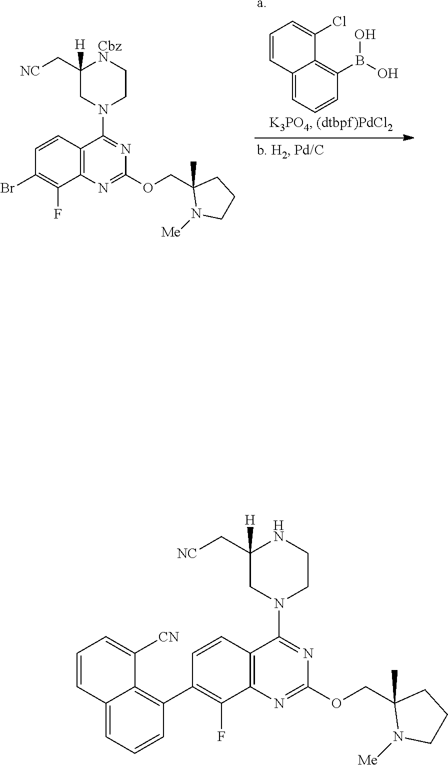

### Step 1: Preparation of benzyl (S)-2-(cyanomethyl)-4-(7-(8-cyanonaphthalen-1-yl)-8-fluoro-2-(((2R,7aS)-2-fluorotetrahydro-1H-pyrrolizin-7a(5H)-yl)methoxy)quinazolin-4-yl)piperazine-1-carboxylate

Benzyl (S)-4-(7-bromo-8-fluoro-2-(((S)-1-methylpyrrolidin-2-yl)methoxy)quinazolin-4-yl)-2-(cyanomethyl)piperazine-1-carboxylate (70 mg, 0.12 mmol) and (8-cyanonaphthalen-1-yl)boronic acid (23 mg, 0.12 mmol) were combined as solids and dissolved in dioxane (4 mL). Potassium phosphate solution (2.0 M, 0.18 mL, 0.35 mmol) was added and nitrogen was sparged through the solution for 15 min. [1,1′-bis(di-tert-butylphosphino)ferrocene]dichloropalladium(II), (dtbpf)PdCl₂, (3.8 mg, 5.9 mol) was added as a solid, and the reaction mixture was heated at 100° C. for 2 h. The mixture was cooled, diluted with EtOAc (10 mL), and washed with water (5 mL) and brine (5 mL). The organic phase was dried over magnesium sulfate, filtered, and concentrated. The crude residue was purified by column chromatography (0→8% MeOH/DCM with 5% NH₄OH) to provide the desired product (40 mg, 0.06 mmol, 51% yield) as an off-white solid. LC/MS (ESI) m/z: [M+H]⁺ calcd for C₃₉H₃₇FN₇O₃ 670.3. found 670.5.

### Step 2: Preparation of 8-(4-((S)-3-(cyanomethyl)piperazin-1-yl)-8-fluoro-2-(((S)-1-methylpyrrolidin-2-yl)methoxy)quinazolin-7-yl)-1-naphthonitrile

Benzyl (S)-2-(cyanomethyl)-4-(7-(8-cyanonaphthalen-1-yl)-8-fluoro-2-(((S)-1-methylpyrrolidin-2-yl)methoxy)quinazolin-4-yl)piperazine-1-carboxylate (40 mg, 0.06 mmol) was dissolved in EtOH (10 mL), and palladium on carbon (10 wt. %, 64 mg, 0.04 mmol) was added. Hydrogen gas was bubbled through the solution, and the reaction mixture was stirred under an atmosphere of hydrogen (balloon) for 1.5 h. The solution was filtered through a pad of Celite™ and concentrated to provide the desired product, which could be used directly in the next step, without additional purification (quantitative yield assumed). LC/MS (ESI) m/z: [M+H]⁺ calcd for C₃₁H₃₁FN₇O 536.3. found 536.4.

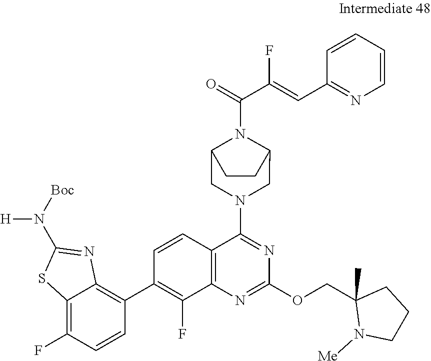 [SMILES: CN1CCC[C@@]1(C)COc1nc(N2CC3CCC(C2)N3C(=O)/C(F)=C/c2ccccn2)c2ccc(-c3ccc(F)c4sc(NC(=O)OC(C)(C)C)nc34)c(F)c2n1]

### tert-butyl (7-fluoro-4-(8-fluoro-4-((1R,5S)-8-((Z)-2-fluoro-3-(pyridin-2-yl)acryloyl)-3,8-diazabicyclo[3.2.1]octan-3-yl)-2-(((S)-1-methylpyrrolidin-2-yl)methoxy)quinazolin-7-yl)benzo[d]thiazol-2-yl)carbamate

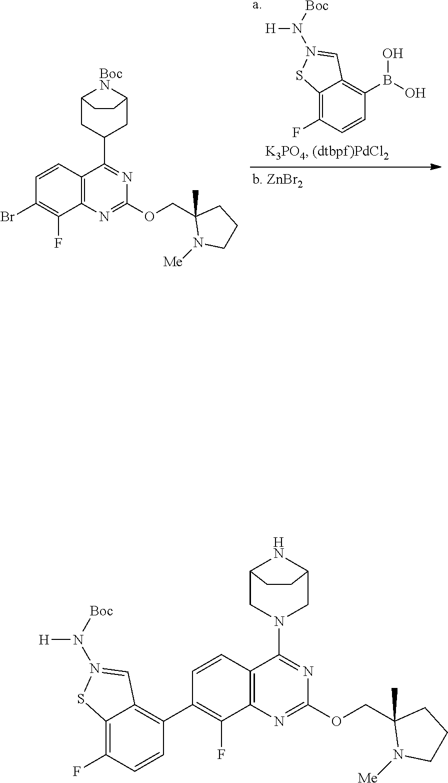

### Step 1: Preparation of tert-butyl (1R,5S)-3-(7-(2-((tert-butoxycarbonyl)amino)-7-fluorobenzo[d]thiazol-4-yl)-8-fluoro-2-(((S)-1-methylpyrrolidin-2-yl)methoxy)quinazolin-4-yl)-3,8-diazabicyclo[3.2.1]octane-8-carboxylate

Tert-butyl (1R,5S)-3-(7-bromo-8-fluoro-2-(((S)-1-methylpyrrolidin-2-yl)methoxy)quinazolin-4-yl)-3,8-diazabicyclo[3.2.1]octane-8-carboxylate (200 mg, 0.36 mmol) and (2-((tert-butoxycarbonyl)amino)-7-fluorobenzo[d]thiazol-4-yl)boronic acid (113 mg, 0.36 mmol were combined as solids and dissolved in dioxane (5 mL). Potassium phosphate solution (2.0 M, 0.55 mL, 1.1 mmol) was added and nitrogen was sparged through the solution for 15 min. [1,1′-bis(di-tert-butylphosphino)ferrocene]dichloropalladium(II), (dtbpf)PdCl₂, (11.8 mg, 0.018 mmol) was added as a solid, and the reaction mixture was heated at 100° C. for 2 h. The mixture was cooled, diluted with EtOAc (10 mL), and washed with water (5 mL) and brine (5 mL). The organic phase was dried over magnesium sulfate, filtered, and concentrated. The crude residue was purified by column chromatography (0→8% MeOH/DCM with 5% NH₄OH) to provide the desired product (60 mg, 0.08 mmol, 22% yield) as an off-white solid. LC/MS (ESI) m/z: [M+H]⁺ calcd for C₃₇H₄₅F₂N₇O₅S 738.3. found 738.6.

### Step 2: Preparation of tert-butyl (4-(4-((1R,5S)-3,8-diazabicyclo[3.2.1]octan-3-yl)-8-fluoro-2-(((S)-1-methylpyrrolidin-2-yl)methoxy)quinazolin-7-yl)-7-fluorobenzo[d]thiazol-2-yl)carbamate

Tert-butyl (1R,5S)-3-(7-(2-((tert-butoxycarbonyl)amino)-7-fluorobenzo[d]thiazol-4-yl)-8-fluoro-2-(((S)-1-methylpyrrolidin-2-yl)methoxy)quinazolin-4-yl)-3,8-diazabicyclo[3.2.1]octane-8-carboxylate (60 mg, 0.08 mmol) was dissolved in DCM (5 mL) and zinc bromide (37 mg, 0.16 mmol) was added in one portion. The resulting suspension was heated at 40° C. for 3 h. Water (10 mL) was added to quench the reaction, and the mixture was stirred for 10 min. EtOAc (10 mL) was added and the layers were separated. The combined organic phase was further washed with water (10 mL) and brine (10 mL), dried over magnesium sulfate, filtered, and concentrated. The crude residue was purified by reverse phase HPLC (column: Xbridge C18, 30 mm×100 mm, 5 m particles; flow rate: 20 mL/min; column temperature: 25° C.; gradient: 100% 5:95 MeCN:H₂O with 10 mM AA/0% 95:5 MeCN:H₂O with 10 mM AA→100% 95:5 MeCN:H₂O with 10 mM AA; λ=220 nm) to provide the desired product as a white solid (30 mg, 0.05 mmol, 58% yield). LC/MS (ESI) m/z: [M+H]⁺ calcd for C₃₂H₃₈F₂N₇O₃S 638.3. found 638.4; ¹H NMR (600 MHz, METHANOL-d₄) δ 7.80 (d, J=8.7 Hz, 1H), 7.52 (dd, J=8.3, 5.4 Hz, 1H), 7.46 (dd, J=8.5, 6.7 Hz, 1H), 7.17 (t, J=8.7 Hz, 1H), 4.54 (br s, 1H), 4.53 (br s, 1H), 4.51 (br d, J=13.3 Hz, 2H), 3.67 (br s, 2H), 3.62 (br d, J=12.7 Hz, 2H), 3.16-3.07 (m, 1H), 2.70 (s, 3H), 2.67-2.60 (m, 1H), 2.25-2.17 (m, 1H), 1.97-1.92 (m, 5H), 1.90-1.82 (m, 5H), 1.56 (s, 9H).

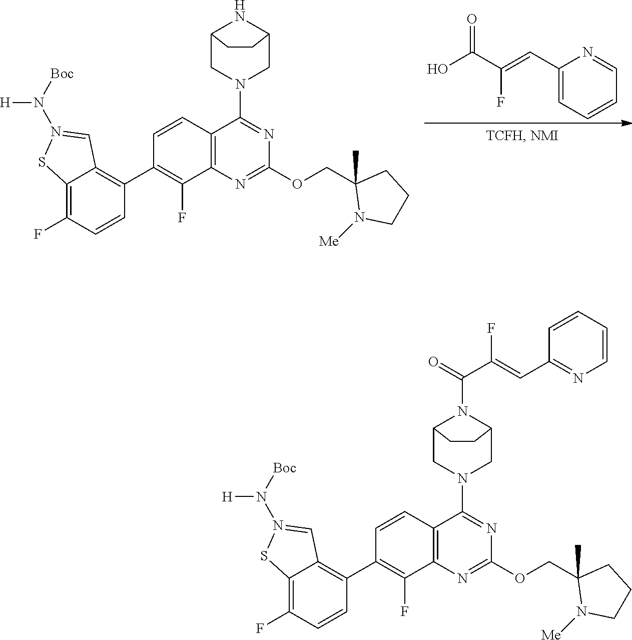

### Step 3: Preparation of tert-butyl (7-fluoro-4-(8-fluoro-4-((1R,5S)-8-((Z)-2-fluoro-3-(pyridin-2-yl)acryloyl)-3,8-diazabicyclo[3.2.1]octan-3-yl)-2-(((S)-1-methylpyrrolidin-2-yl)methoxy)quinazolin-7-yl)benzo[d]thiazol-2-yl)carbamate

Tert-butyl (4-(4-((1R,5S)-3,8-diazabicyclo[3.2.1]octan-3-yl)-8-fluoro-2-(((S)-1-methylpyrrolidin-2-yl)methoxy)quinazolin-7-yl)-7-fluorobenzo[d]thiazol-2-yl)carbamate (30 mg, 0.05 mmol) and (Z)-2-fluoro-3-(pyridin-2-yl)acrylic acid (8 mg, 0.05 mmol) were combined as solids and dissolved in DMF (1.0 mL). 1-Methylimidazole (40 μL, 0.5 mmol) was added followed by chloro-N,N,N′,N′-tetramethylformamidinium hexafluorophosphate (7 mg, 0.05 mmol). The reaction mixture was stirred at room temperature for 5 min. EtOAc (10 mL) and water (10 mL) were added and the layers were separated. The organic phase was washed with brine (5 mL), dried over magnesium sulfate, filtered, and concentrated. The crude residue was used directly in the next step, without additional purification (quantitative yield assumed). LC/MS (ESI) m/z: [M+H]⁺ calcd for C₄₀H₄₂F₃N₈O₄S 786.3. found 786.3.

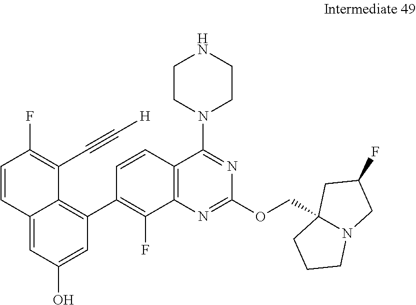 [SMILES: C#Cc1c(F)ccc2cc(O)cc(-c3ccc4c(N5CCNCC5)nc(OC[C@@]56CCCN5C[C@H](F)C6)nc4c3F)c12]

### 5-ethynyl-6-fluoro-4-(8-fluoro-2-(((2R,7aS)-2-fluorotetrahydro-1H-pyrrolizin-7a(5H)-yl)methoxy)-4-(piperazin-1-yl)quinazolin-7-yl)naphthalen-2-ol

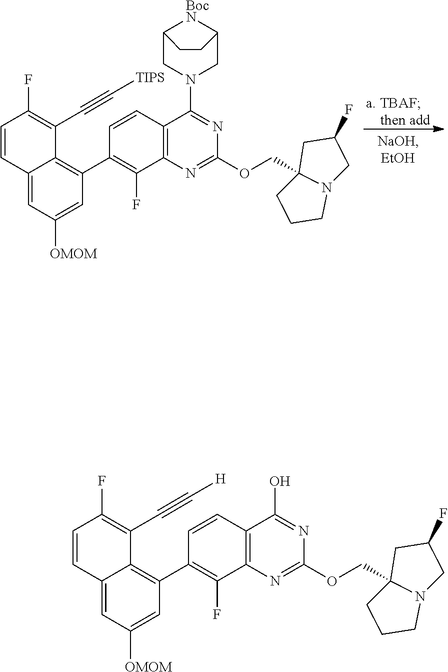

### Step 1: Preparation of 7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-ol

tert-Butyl (1R,5S)-3-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (prepared according to the procedure described in WO2022/061251, 430 mg, 0.58 mmol) was dissolved in THE (6 mL), and TBAF solution (1.0 M in THF, 0.58 mL, 0.58 mmol) was added dropwise. The reaction mixture was stirred at room temperature for 5 minutes and was concentrated. The residue was taken up in EtOH (6 mL), and sodium hydroxide solution (1.0 M, 1.7 mL, 1.7 mmol) was added. The reaction mixture was heated at 60° C. for 16 h and at 70° C. for an additional 16 h. EtOAc (10 mL) and water (10 mL) were added, and the layers were separated. The aqueous layer was extracted with EtOAc (2×5 mL). The combined organic phases were washed with brine (5 mL), dried over magnesium sulfate, filtered, and concentrated. The crude residue was purified by column chromatography (0→10% MeOH/DCM) to provide the desired product (256 mg, 0.23 mmol, 40% yield); LC/MS (ESI) m/z: [M+H]⁺ calcd for C₃₀H₂₇F₃N₃O₄ 550.2. found 550.1; ¹H NMR (600 MHz, DMSO-d₆) δ 8.12-8.04 (m, 1H), 7.82 (br d, J=8.0 Hz, 1H), 7.69 (br s, 1H), 7.53 (br t, J=8.8 Hz, 1H), 7.31-7.22 (m, 2H), 5.44-5.22 (m, 2H), 5.31 (d, J=56.7 Hz, 1H), 4.23-4.07 (m, 2H), 4.02 (s, 1H), 3.44 (s, 3H), 3.40-3.23 (m, 1H), 3.25-3.01 (m, 3H), 2.84 (br d, J=5.8 Hz, 1H), 2.24-1.94 (m, 3H), 1.89-1.76 (m, 3H).

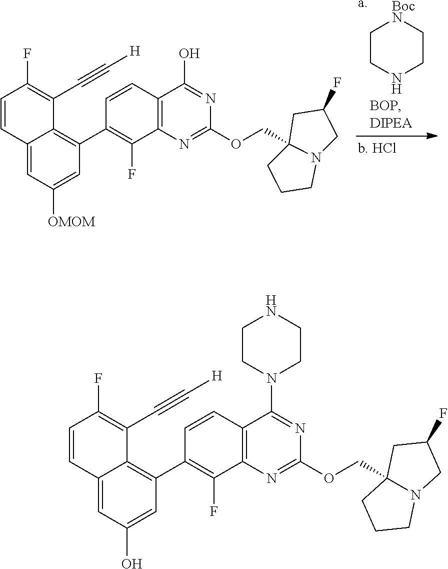

### Step 2: Preparation of tert-butyl 4-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)piperazine-1-carboxylate

7-(8-Ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-ol (20 mg, 0.04 mmol) was dissolved in MeCN (0.5 mL). Tert-butyl piperazine-1-carboxylate (14 mg, 0.07 mmol) and DIPEA (0.03 mL, 0.18 mmol) were added, and the reaction mixture was heated at 60° C. for 1 h. The solution was concentrated, and the crude residue was used directly in the next step, without additional purification. LC/MS (ESI) m/z: [M+H]⁺ calcd for C₃₉H₄₃F₃N₅O₅ 718.3. found 718.2.

### Step 3: Preparation of 5-ethynyl-6-fluoro-4-(8-fluoro-2-(((2R,7aS)-2-fluorotetrahydro-1H-pyrrolizin-7a(5H)-yl)methoxy)-4-(piperazin-1-yl)quinazolin-7-yl)naphthalen-2-ol

tert-Butyl 4-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)piperazine-1-carboxylate (26 mg, 0.04 mmol) was dissolved in dioxane (0.5 mL) and methanol (0.5 mL). HCl solution (4.0 M in dioxane, 0.1 mL, 0.4 mmol) was added dropwise, and the reaction mixture was stirred for 2 h. The solution was directly purified by reverse phase HPLC (column: Xbridge C18, 19 mm×200 mm, 5 m particles; flow rate: 20 mL/min; column temperature: 25° C.; gradient: 94% 5:95 MeCN:H₂O with 0.1% TFA/6% 95:5 MeCN:H₂O with 0.1% TFA→100% 95:5 MeCN:H₂O with 0.1% TFA; λ=220 nm) to provide the desired product as the bis TFA salt (21.1 mg, 0.03 mmol, 73% yield). LC/MS (ESI) m/z: [M+H]⁺ calcd for C₃₂H₃₁F₃N₅O₂ 574.2; found 574.3; ¹H NMR (500 MHz, DMSO-d₆) δ 9.21-9.03 (m, 1H), 8.01-7.93 (m, 1H), 7.86 (d, J=8.4 Hz, 1H), 7.47 (t, J=9.0 Hz, 1H), 7.38 (d, J=2.3 Hz, 1H), 7.37-7.33 (m, 1H), 7.09 (d, J=2.3 Hz, 1H), 5.56 (d, J=52.2 Hz, 1H), 4.65-4.54 (m, 2H), 4.00-3.91 (m, 4H), 3.88-3.72 (m, 3H), 3.53-3.42 (m, 5H), 3.42-3.27 (m, 1H), 2.66-2.52 (m, 3H), 2.38-2.28 (m, 1H), 2.25-2.12 (m, 2H), 2.10-1.97 (m, 1H).

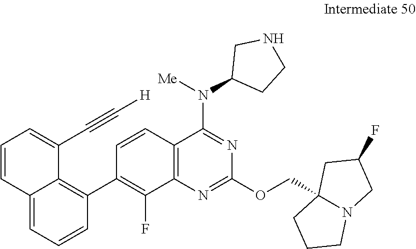 [SMILES: C#Cc1cccc2cccc(-c3ccc4c(N(C)[C@@H]5CCNC5)nc(OC[C@@]56CCCN5C[C@H](F)C6)nc4c3F)c12]

### 7-(8-ethynylnaphthalen-1-yl)-8-fluoro-2-(((2R,7aS)-2-fluorotetrahydro-1H-pyrrolizin-7a(5H)-yl)methoxy)-N-methyl-N-((R)-pyrrolidin-3-yl)quinazolin-4-amine

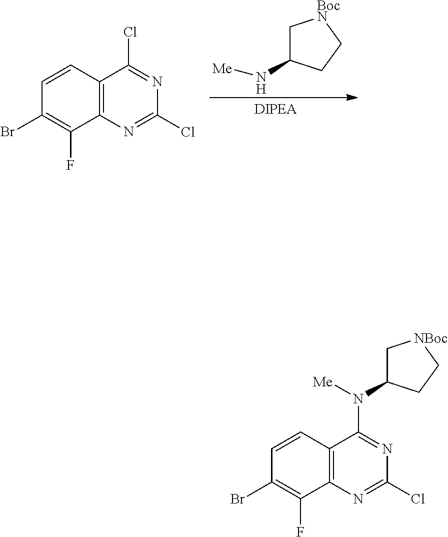 [SMILES: Fc1c(Br)ccc2c(Cl)nc(Cl)nc12 ; CN[C@@H]1CCN(C(=O)OC(C)(C)C)C1 ; CN(c1nc(Cl)nc2c(F)c(Br)ccc12)[C@@H]1CCN(C(=O)OC(C)(C)C)C1]

### Step 1: Preparation of tert-butyl (R)-3-((7-bromo-2-chloro-8-fluoroquinazolin-4-yl)(methyl)amino)pyrrolidine-1-carboxylate

7-Bromo-2,4-dichloro-8-fluoroquinazoline (450 mg, 1.52 mmol) was suspended in DCM (5 mL), and the mixture was cooled to 0° C. DIPEA (0.40 mL, 2.28 mmol) was added followed by tert-butyl (R)-3-(methylamino)pyrrolidine-1-carboxylate (335 mg, 1.67 mmol), and the solution was allowed to warm to room temperature. The reaction mixture was stirred at room temperature for 2 h before being directly concentrated and used in the next step, without additional purification (quantitative yield assumed). LC/MS (ESI) m/z: [M+H]⁺ calcd for C₁₈H₂₂BrClFN₄O₂ 459.1. found 459.0.

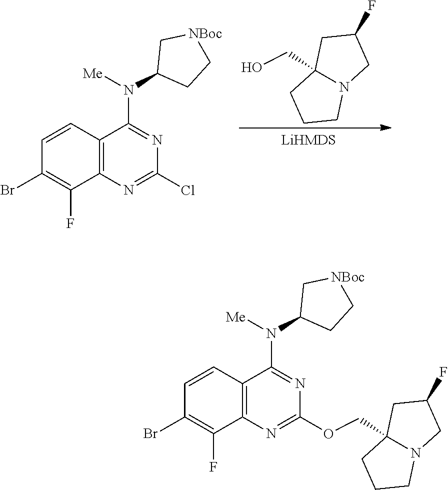 [SMILES: CN(c1nc(Cl)nc2c(F)c(Br)ccc12)[C@@H]1CCN(C(=O)OC(C)(C)C)C1 ; OC[C@@]12CCCN1C[C@H](F)C2 ; CN(c1nc(OC[C@@]23CCCN2C[C@H](F)C3)nc2c(F)c(Br)ccc12)[C@@H]1CCN(C(=O)OC(C)(C)C)C1]

### Step 2: Preparation of tert-butyl (R)-3-((7-bromo-8-fluoro-2-(((2R,7aS)-2-fluorotetrahydro-1H-pyrrolizin-7a(5H)-yl)methoxy)quinazolin-4-yl)(methyl)amino)pyrrolidine-1-carboxylate

((2R,7aS)-2-Fluorotetrahydro-1H-pyrrolizin-7a(5H)-yl)methanol (242 mg, 1.52 mmol) was dissolved in THE (5.0 mL), and LiHMDS solution (1.0 M in THF, 3.8 mL, 3.8 mmol) was added dropwise. After stirring for 5 min, tert-butyl (R)-3-((7-bromo-2-chloro-8-fluoroquinazolin-4-yl)(methyl)amino)pyrrolidine-1-carboxylate (700 mg, 1.52 mmol) was added as a solid, and the reaction mixture was heated at 65° C. for 48 h.

The reaction mixture was directly concentrated, and the crude residue was purified by column chromatography (0→100% EtOAc with 5% Et3N/hexanes) to provide the desired product (500 mg, 0.858 mmol, 56.4% yield over two steps). LC/MS (ESI) m/z: [M+H]⁺ calcd for C₂₆H₃₅BrClF₂N₅O₃ 582.2. found 582.2; ¹H NMR (500 MHz, CDCl₃) δ 7.53 (dd, J=9.0, 1.5 Hz, 1H), 7.32 (dd, J=9.0, 6.3 Hz, 1H), 5.27 (d, J=53.8 Hz, 1H), 5.13-5.04 (m, 1H), 4.26 (d, J=10.3 Hz, 1H), 4.15 (d, J=10.3 Hz, 1H), 3.88-3.79 (m, 1H), 3.72-3.56 (m, 1H), 3.50-3.30 (m, 2H), 3.28-3.14 (m, 3H), 3.24 (s, 3H), 3.00-2.93 (m, 1H), 2.29-2.10 (m, 5H), 1.98-1.83 (m, 3H), 1.48 (s, 9H).

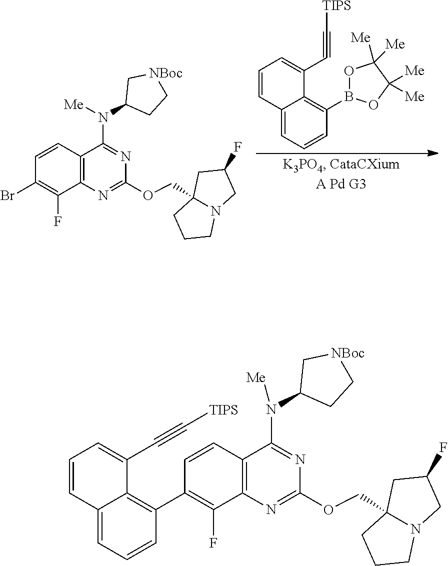

### Step 3 Preparation of tert-butyl (R)-3-((8-fluoro-2-((2R,7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)(methyl)amino)pyrrolidine-1-carboxylate

tert-Butyl (S)-3-((7-bromo-8-fluoro-2-(((2R,7aS)-2-fluorotetrahydro-1H-pyrrolizin-7a(5H)-yl)methoxy)quinazolin-4-yl)(methyl)amino)pyrrolidine-1-carboxylate (50 mg, 0.086 mmol), triisopropyl((8-(4,4,5,5-tetramethyl-1,3,2-dioxaborolan-2-yl)naphthalen-1-yl)ethynyl)silane (37 mg, 0.086 mmol), and methanesulfonato(diadamantyl-n-butylphosphino)-2′-amino-1,1′-biphenyl-2-yl)palladium(II) dichloromethane adduct (cataCXium A Palladacycle Gen3) (3.1 mg, 4.3 μmol) were combined as solids in a microwave vial. The vial was sealed. The atmosphere was evacuated and replaced with nitrogen. This process was performed three times. Degassed dioxane (2 mL) was added, and the reaction mixture was heated at 100° C. for 2 h. The solution was directly concentrated, and the crude residue was purified by column chromatography (0→10% MeOH/DCM) to provide the desired product (65 mg, 0.080 mmol, 93% yield). LC/MS (ESI) m/z: [M+H]⁺ calcd for C₄₇H₆₂F₂N₅O₃Si 810.5. found 810.9; ¹H NMR (500 MHz, CD₃OD) δ 8.04-8.00 (m, 2H), 7.98-7.91 (m, 1H), 7.79-7.76 (m, 1H), 7.64-7.58 (m, 1H), 7.54-7.48 (m, 1H), 7.46-7.41 (m, 1H), 7.37-7.28 (m, 1H), 5.32 (d, J=55.4 Hz, 1H), 4.41-4.14 (m, 2H), 3.97-3.60 (m, 2H), 3.51-3.42 (m, 3H), 3.09-2.98 (m, 2H), 2.51-2.12 (m, 6H), 2.09-1.85 (m, 4H), 1.50 (s, 9H), 1.49-1.48 (m, 3H), 0.95-0.86 (m, 18H), 0.63-0.52 (m, 3H).

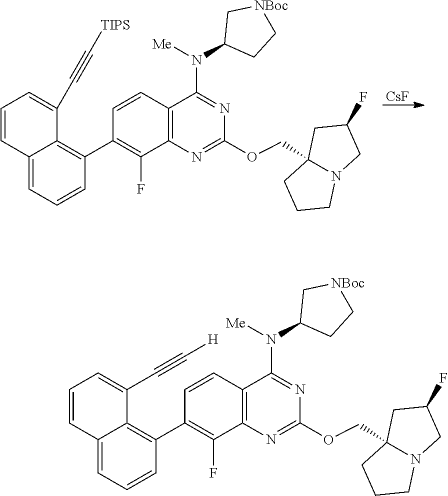

### Step 4: Preparation of tert-butyl (R)-3-((7-(8-ethynylnaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

tert-Butyl (S)-3-((8-fluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazolin-4-yl)(methyl)amino)pyrrolidine-1-carboxylate (60 mg, 0.074 mmol) was dissolved in DMF (2 mL), and cesium fluoride (34 mg, 0.22 mmol) was added as a solid. The mixture was heated at 60° C. for 1 h. The suspension was cooled to room temperature and diluted with EtOAc (5 mL) and water (5 mL). The layers were separated, and the organic phase was further washed with brine (5 mL), dried over magnesium sulfate, filtered, and concentrated. The crude residue was used directly in the next step, without additional purification (quantitative yield assumed). LC/MS (ESI) m/z: [M+H]⁺ calcd for C₃₈H₄₂F₂N₅O₃ 654.3 found 654.4.

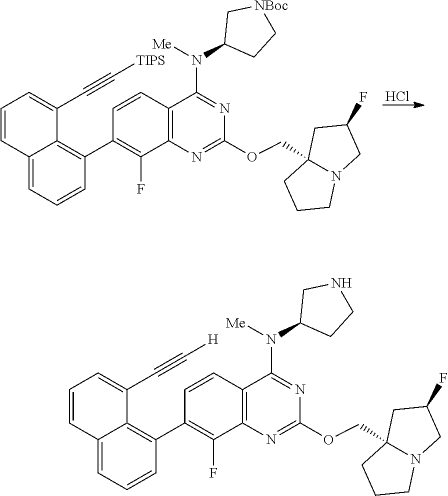

### Step 5: Preparation of 7-(8-ethynylnaphthalen-1-yl)-8-fluoro-2-(((2R,7aS)-2-fluorotetrahydro-1H-pyrrolizin-7a(5H)-yl)methoxy)-N-methyl-N-((R)-pyrrolidin-3-yl)pyrido[4,3-d]pyrimidin-4-amine

tert-Butyl (S)-3-((7-(8-ethynylnaphthalen-1-yl)-8-fluoro-2-(((2R,7aS)-2-fluoro-tetrahydro-1H-pyrrolizin-7a(5H)-yl)methoxy)quinazolin-4-yl)(methyl)amino)pyrrolidine-1-carboxylate (48 mg, 0.073 mmol) was dissolved in DMF (0.5 mL), and a solution of HCL (4.0 M in dioxane, 0.25 mL, 1.0 mmol) was added dropwise. The reaction mixture was stirred at room temperature for 1 h. The solution was directly concentrated, and the crude residue was evaporated from DCM (2×5 mL) to provide the desired product as its HCl salt (40 mg, 0.068 mmol, 92% yield over two steps). LC/MS (ESI) m/z: [M+H]⁺ calcd for C₃₃H₃₄F₂N₅O 554.3. found 554.2.

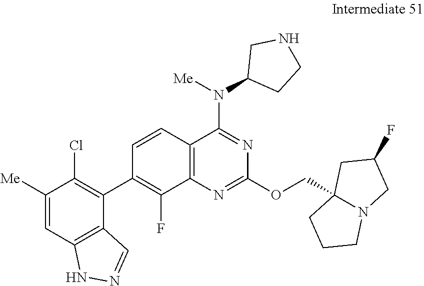 [SMILES: Cc1cc2[nH]ncc2c(-c2ccc3c(N(C)[C@@H]4CCNC4)nc(OC[C@@]45CCCN4C[C@H](F)C5)nc3c2F)c1Cl]

### 7-(5-chloro-6-methyl-1H-indazol-4-yl)-8-fluoro-2-(((2R,7aS)-2-fluorotetrahydro-1H-pyrrolizin-7a(5H)-yl)methoxy)-N-methyl-N-((R)-pyrrolidin-3-yl)quinazolin-4-amine

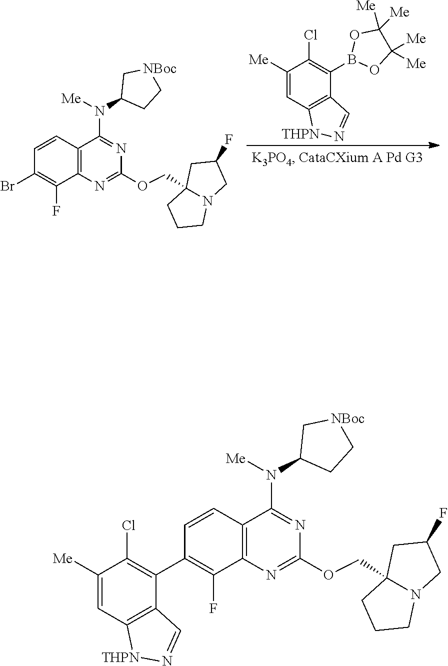

### Step 1: Preparation of tert-butyl (3R)-3-((7-(5-chloro-6-methyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)(methyl)amino)pyrrolidine-1-carboxylate

tert-Butyl (S)-3-((7-bromo-8-fluoro-2-(((2R,7aS)-2-fluorotetrahydro-1H-pyrrolizin-7a(5H)-yl)methoxy)quinazolin-4-yl)(methyl)amino)pyrrolidine-1-carboxylate (50 mg, 0.086 mmol), 5-chloro-6-methyl-1-(tetrahydro-2H-pyran-2-yl)-4-(4,4,5,5-tetramethyl-1,3,2-dioxaborolan-2-yl)-1H-indazole (commercially available, 32 mg, 0.086 mmol), and methanesulfonato(diadamantyl-n-butylphosphino)-2′-amino-1,1′-biphenyl-2-yl)palladium(II) dichloromethane adduct (cataCXium A Palladacycle Gen3) (3.1 mg, 4.3 μmol) were combined as solids in a microwave vial. The vial was sealed. The atmosphere was evacuated and replaced with nitrogen. This process was performed three times. Degassed dioxane (2 mL) was added, and the reaction mixture was heated at 100° C. for 2 h. The solution was directly concentrated, and the crude residue was purified by column chromatography (0→8% MeOH/DCM) to provide the desired product (51 mg, 0.067 mmol, 79% yield). LC/MS (ESI) m/z: [M+H]⁺ calcd for C₃₉H₄₉ClF₂N₇O₄ 752.3. found 752.3.

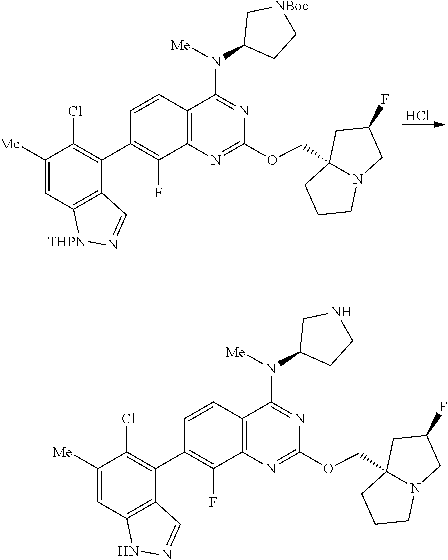

### Step 2: Preparation of 7-(5-chloro-6-methyl-1H-indazol-4-yl)-8-fluoro-2-(((2R,7aS)-2-fluorotetrahydro-1H-pyrrolizin-7a(5H)-yl)methoxy)-N-methyl-N-((R)-pyrrolidin-3-yl)quinazolin-4-amine

tert-Butyl (3S)-3-((7-(5-chloro-6-methyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)(methyl)amino)pyrrolidine-1-carboxylate (51 mg, 0.067 mmol) was dissolved in dioxane (0.5 mL), and a solution of HCL (4.0 M in dioxane, 0.25 mL, 1.0 mmol) was added dropwise. The reaction mixture was stirred at room temperature for 1 h. The solution was directly concentrated, and the crude residue was evaporated from DCM (2×5 mL) to provide the desired product as its HCl salt (30 mg, 0.046 mmol, 69% yield). LC/MS (ESI) m/z: [M+H]⁺ calcd for C₂₉H₃₃ClF₂N₇O 568.2. found 568.2.

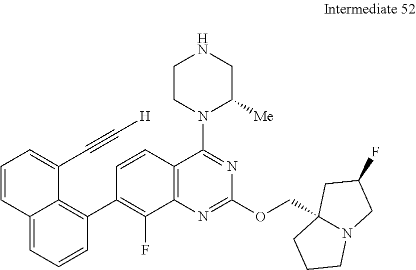 [SMILES: C#Cc1cccc2cccc(-c3ccc4c(N5CCNC[C@@H]5C)nc(OC[C@@]56CCCN5C[C@H](F)C6)nc4c3F)c12]

### 7-(8-ethynylnaphthalen-1-yl)-8-fluoro-2-(((2R,7aS)-2-fluorotetrahydro-1H-pyrrolizin-7a(5H)-yl)methoxy)-4-((S)-2-methylpiperazin-1-yl)quinazoline

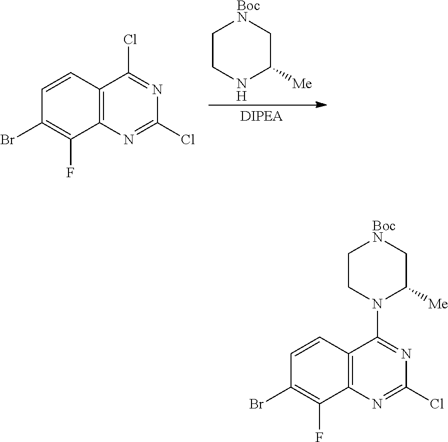 [SMILES: Fc1c(Br)ccc2c(Cl)nc(Cl)nc12 ; C[C@H]1CN(C(=O)OC(C)(C)C)CCN1 ; C[C@H]1CN(C(=O)OC(C)(C)C)CCN1c1nc(Cl)nc2c(F)c(Br)ccc12]

### Step 1: Preparation of tert-butyl (S)-4-(7-bromo-2-chloro-8-fluoroquinazolin-4-yl)-3-methylpiperazine-1-carboxylate

7-Bromo-2,4-dichloro-8-fluoroquinazoline (500 mg, 1.69 mmol) was dissolved in dioxane (15 mL). DIPEA (0.9 mL, 5.1 mmol) was added dropwise followed by tert-butyl (S)-3-methylpiperazine-1-carboxylate (340 mg, 1.69 mmol). The reaction mixture was stirred at room temperature for 2 h. The solution was concentrated, and the crude residue was purified by column chromatography (5→50% EtOAc/hexanes) to provide the desired product (701 mg, 1.53 mmol, 90% yield) as a white solid. LC/MS (ESI) m/z: [M+H]⁺ calcd for C₁₈H₂₂BrClFN₄O₂ 459.1. found 459.1; ¹H NMR (500 MHz, CDCl₃) δ 7.53 (dd, J=9.1, 6.1 Hz, 1H), 7.45 (dd, J=9.1, 0.7 Hz, 1H), 4.65 (s, 1H), 4.30-3.82 (m, 3H), 3.69-3.53 (m, 1H), 3.37-2.86 (m, 2H), 1.49 (s, 9H), 1.44 (d, J=6.8 Hz, 3H).

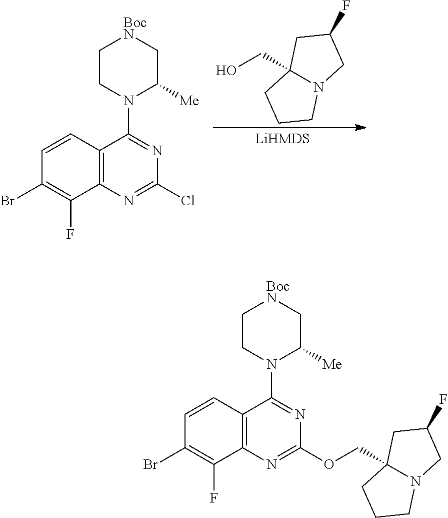 [SMILES: C[C@H]1CN(C(=O)OC(C)(C)C)CCN1c1nc(Cl)nc2c(F)c(Br)ccc12 ; OC[C@@]12CCCN1C[C@H](F)C2 ; C[C@H]1CN(C(=O)OC(C)(C)C)CCN1c1nc(OC[C@@]23CCCN2C[C@H](F)C3)nc2c(F)c(Br)ccc12]

### Step 2: Preparation of tert-butyl (S)-4-(7-bromo-8-fluoro-2-(((2R,7aS)-2-fluorotetrahydro-1H-pyrrolizin-7a(5H)-yl)methoxy)quinazolin-4-yl)-3-methylpiperazine-1-carboxylate

tert-Butyl (S)-4-(7-bromo-2-chloro-8-fluoroquinazolin-4-yl)-3-methylpiperazine-1-carboxylate (500 mg, 1.09 mmol) and ((2R,7aS)-2-fluorotetrahydro-1H-pyrrolizin-7a(5H)-yl)methanol, HCl salt (234 mg, 1.20 mmol) were dissolved in THF (12 mL), and LiHMDS solution (1.0 M in THF, 2.4 mL, 2.4 mmol) was added dropwise. The reaction mixture was heated at 65° C. for 16 h. The solution was partially concentrated, and the crude residue was purified by column chromatography (50→100% EtOAc with 5% Et3N/hexanes) to provide the desired product (415 mg, 0.71 mmol, 66% yield) as a yellow solid. LC/MS (ESI) m/z: [M+H]⁺ calcd for C₂₆H₃₅BrF₂N₅O₃ 582.2. found 582.1; ¹H NMR (500 MHz, CD₃OD) δ 7.63 (dd, J=9.0, 1.3 Hz, 1H), 7.46 (dd, J=9.0, 6.4 Hz, 1H), 5.30 (d, J=54.6 Hz, 1H), 4.79-4.69 (m, 1H), 4.27 (d, J=10.5 Hz, 1H), 4.20 (d, J=10.5 Hz, 1H), 4.23-4.15 (m, 1H), 4.14-4.03 (m, 1H), 3.94-3.87 (m, 1H), 3.69-3.58 (m, 1H), 3.30-3.13 (m, 5H), 3.06-2.96 (m, 1H), 2.39-2.09 (m, 3H), 2.02-1.85 (m, 3H), 1.49 (s, 9H), 1.40 (d, J=6.7 Hz, 3H).

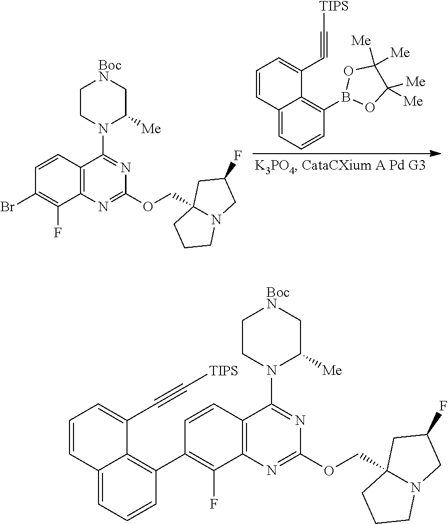

### Step 3: Preparation of tert-butyl (S)-4-(8-fluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazolin-4-yl)-3-methylpiperazine-1-carboxylate

tert-Butyl (S)-4-(7-bromo-8-fluoro-2-(((2R,7aS)-2-fluorotetrahydro-1H-pyrrolizin-7a(5H)-yl)methoxy)quinazolin-4-yl)-3-methylpiperazine-1-carboxylate (150 mg, 0.258 mmol), triisopropyl((8-(4,4,5,5-tetramethyl-1,3,2-dioxaborolan-2-yl)naphthalen-1-yl)ethynyl)silane (112 mg, 0.26 mmol), and methanesulfonato(diadamantyl-n-butylphosphino)-2′-amino-1,1′-biphenyl-2-yl)palladium(II) dichloromethane adduct, [cataCXium A Palladacycle Gen. 3](9.4 mg, 0.013 mmol) were combined as solids in a microwave vial. The vial was sealed. The atmosphere was evacuated and replaced with nitrogen. This process was performed three times. Degassed dioxane (2 mL) and potassium phosphate solution (2.0 M in water, 390 μL, 0.77 mmol) were added, and the reaction mixture was heated at 100° C. in the microwave for 2 h. The reaction mixture was directly concentrated, and the crude residue was purified by column chromatography (0→10% MeOH/DCM) to provide the desired product (190 mg, 0.024 mmol, 91% yield). LC/MS (ESI) m/z: [M+H]⁺ calcd for C₄₇H₆₂F₂N₅O₃Si 810.5. found 810.3; ¹H NMR (500 MHz, CD₃OD) δ 8.07-7.98 (m, 2H), 7.80-7.69 (m, 2H), 7.64-7.58 (m, 1H), 7.54-7.48 (m, 1H), 7.45-7.39 (m, 1H), 7.37-7.30 (m, 1H), 5.30 (d, J=55.0 Hz, 1H), 4.96-4.87 (m, 1H), 4.34-4.07 (m, 4H), 4.00-3.91 (m, 1H), 3.84-3.58 (m, 1H), 3.28-3.13 (m, 5H), 3.07-2.97 (m, 1H), 2.40-2.12 (m, 3H), 2.10-1.93 (m, 3H), 1.54 (d, J=6.7 Hz, 1.5H), 1.52-1.49 (m, 9H), 1.37 (d, J=6.6 Hz, 1.5H), 0.93-0.85 (m, 18H), 0.59-0.50 (m, 3H).

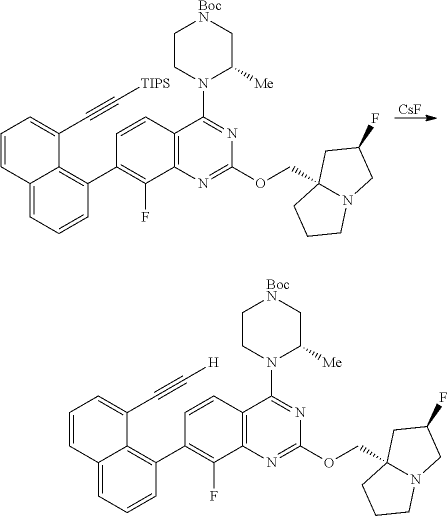

### Step 4: Preparation of tert-butyl (S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-3-methylpiperazine-1-carboxylate

tert-Butyl (S)-4-(8-fluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazolin-4-yl)-3-methylpiperazine-1-carboxylate (190 mg, 0.235 mmol) was dissolved in DMF (3 mL), and cesium fluoride (107 mg, 0.70 mmol) was added as a solid. The mixture was heated at 60° C. for 1 h. The suspension was cooled to room temperature and diluted with EtOAc (10 mL) and water (5 mL). The layers were separated, and the organic phase was further washed with brine (5 mL), dried over magnesium sulfate, filtered, and concentrated. The crude residue was used directly in the next step, without additional purification (quantitative yield assumed). LC/MS (ESI) m/z: [M+H]⁺ calcd for C₃₈H₄₂F₂N₅O₃ 654.3. found 654.5.

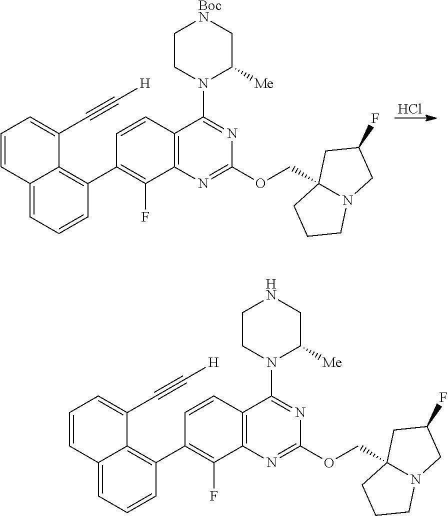 [SMILES: C#Cc1cccc2cccc(-c3ccc4c(N5CCN(C(=O)OC(C)(C)C)C[C@@H]5C)nc(OC[C@@]56CCCN5C[C@H](F)C6)nc4c3F)c12 ; Cl ; C#Cc1cccc2cccc(-c3ccc4c(N5CCNC[C@@H]5C)nc(OC[C@@]56CCCN5C[C@H](F)C6)nc4c3F)c12]

### Step 5: Preparation of 7-(8-ethynylnaphthalen-1-yl)-8-fluoro-2-(((2R,7aS)-2-fluorotetrahydro-1H-pyrrolizin-7a(5H)-yl)methoxy)-4-((S)-2-methylpiperazin-1-yl)quinazoline

tert-Butyl (S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-3-methylpiperazine-1-carboxylate (140 mg, 0.214 mmol) was dissolved in dioxane (1.0 mL), and a solution of HCL (4.0 M in dioxane, 0.25 mL, 1.0 mmol) was added dropwise. The reaction mixture was stirred at room temperature for 1 h. The solution was directly concentrated, and the crude residue was evaporated from DCM (2×5 mL) to provide the desired product as its HCl salt (quantitative yield assumed). LC/MS (ESI) m/z: [M+H]⁻ calcd for C₃₃H₃₄F₂N₅O 554.3. found 554.2.

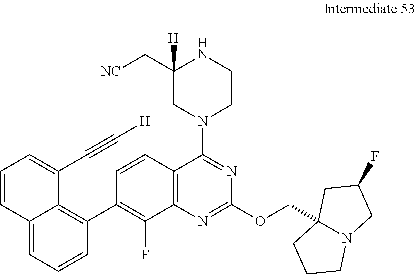

### 2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile

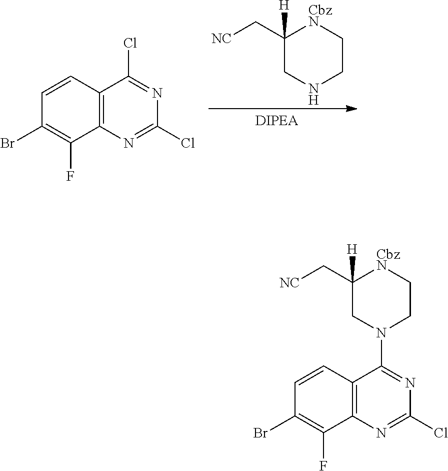 [SMILES: Fc1c(Br)ccc2c(Cl)nc(Cl)nc12 ; N#CC[C@H]1CNCCN1C(=O)OCc1ccccc1 ; N#CC[C@H]1CN(c2nc(Cl)nc3c(F)c(Br)ccc23)CCN1C(=O)OCc1ccccc1]

### Step 1: Preparation of benzyl (S)-4-(7-bromo-2-chloro-8-fluoroquinazolin-4-yl)-2-(cyanomethyl)piperazine-1-carboxylate

7-Bromo-2,4-dichloro-8-fluoroquinazoline (500 mg, 1.69 mmol) was dissolved in dioxane (15 mL). DIPEA (0.9 mL, 5.1 mmol) was added dropwise followed by tert-butyl benzyl (S)-2-(cyanomethyl)piperazine-1-carboxylate (438 mg, 1.69 mmol). The reaction mixture was stirred at room temperature for 2 h. The solution was concentrated, and the crude residue was purified by column chromatography (5→50% EtOAc/hexanes) to provide the desired product (790 mg, 1.53 mmol, 90% yield) as a white solid. LC/MS (ESI) m/z: [M+H]⁺ calcd for C₂₂H₁₉BrClFN₅O₂ 518.0. found 518.1; ¹H NMR (500 MHz, CD₃OD) δ 7.81 (dd, J=9.2, 1.5 Hz, 1H), 7.69 (dd, J=9.2, 6.4 Hz, 1H), 7.44-7.29 (m, 5H), 5.24-5.14 (m, 2H), 4.79-4.73 (m, 1H), 4.50-4.34 (m, 2H), 4.15-4.08 (m, 1H), 3.84-3.71 (m, 1H), 3.70-3.61 (m, 1H), 3.59-3.47 (m, 1H), 3.05-2.88 (m, 2H).

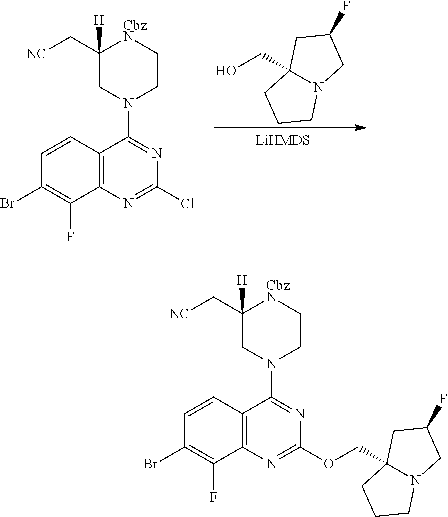

### Step 2: Preparation of benzyl (S)-4-(7-bromo-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate

Benzyl (S)-4-(7-bromo-2-chloro-8-fluoroquinazolin-4-yl)-2-(cyanomethyl)-piperazine-1-carboxylate (120 mg, 0.23 mmol) and ((2R,7aS)-2-fluorotetrahydro-1H-pyrrolizin-7a(5H)-yl)methanol, HCl salt (50 mg, 0.25 mmol) were dissolved in THE (5 mL), and LiHMDS solution (1.0 M in THF, 0.51 mL, 0.51 mmol) was added dropwise. The reaction mixture was heated at 65° C. for 16 h. The solution was partially concentrated, and the crude residue was purified by column chromatography (50→100% EtOAc with 5% Et3N/hexanes) to provide the desired product (90 mg, 0.14 mmol, 61% yield) as a yellow solid. LC/MS (ESI) m/z: [M+H]⁺ calcd for C₃₀H₃₂BrF₂N₆O₃ 641.2. found 641.3; ¹H NMR (500 MHz, CD₃OD) δ 7.72 (dd, J=9.0, 1.4 Hz, 1H), 7.48 (dd, J=9.0, 6.4 Hz, 1H), 7.41-7.32 (m, 5H), 5.30 (d, J=54.0 Hz, 1H), 5.18 (s, 2H), 4.79-4.71 (m, 1H), 4.44-4.30 (m, 2H), 4.28 (d, J=10.4 Hz, 1H), 4.23 (d, J=10.4 Hz, 1H), 3.73-3.66 (m, 1H), 3.56-3.49 (m, 2H), 3.26-3.14 (m, 4H), 3.06-2.94 (m, 3H), 2.39-2.09 (m, 3H), 2.02-1.85 (m, 3H).

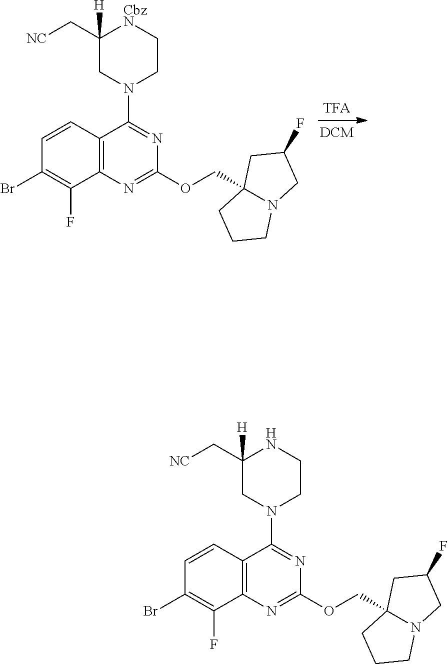 [SMILES: N#CC[C@H]1CN(c2nc(OC[C@@]34CCCN3C[C@H](F)C4)nc3c(F)c(Br)ccc23)CCN1C(=O)OCc1ccccc1 ; N#CC[C@H]1CN(c2nc(OC[C@@]34CCCN3C[C@H](F)C4)nc3c(F)c(Br)ccc23)CCN1]

### Step 3: Preparation of 2-((S)-4-(7-bromo-8-fluoro-2-(((2R,7aS)-2-fluorotetrahydro-1H-pyrrolizin-7a(5H)-yl)methoxy)quinazolin-4-yl)piperazin-2-yl)acetonitrile

Benzyl (S)-4-(7-bromo-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate (100 mg, 0.156 mmol) was placed in a microwave vial, and DCM (1 mL) and TFA (0.25 mL) were added. The reaction mixture was heated at 90° C. in the microwave for 3 h. The solution was directly concentrated, and the residue was evaporated from DCM (3×5 mL). The crude material was used directly in the next step, without additional purification. LC/MS (ESI) m/z: [M+H]⁺ calcd for C₂₂H₂₆BrF₂N₆O 507.1. found 507.1.

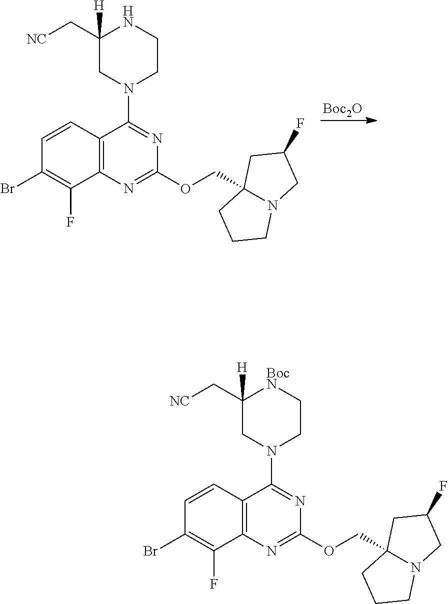 [SMILES: "Boc2O" ; N#CC[C@H]1CN(c2nc(OC[C@@]34CCCN3C[C@H](F)C4)nc3c(F)c(Br)ccc23)CCN1 ; CC(C)(C)OC(=O)N1CCN(c2nc(OC[C@@]34CCCN3C[C@H](F)C4)nc3c(F)c(Br)ccc23)C[C@@H]1CC#N]

### Step 4: Preparation of tert-butyl (S)-4-(7-bromo-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate

2-((S)-4-(7-bromo-8-fluoro-2-(((2R,7aS)-2-fluorotetrahydro-1H-pyrrolizin-7a(5H)-yl)methoxy)quinazolin-4-yl)piperazin-2-yl)acetonitrile (110 mg, 0.217 mmol) was dissolved in DCM (15 mL), and triethylamine (0.09 mL, 0.65 mmol) was added followed by di-tert-butyl dicarbonate (0.08 mL, 0.325 mmol). The reaction mixture was stirred at room temperature for 4 h. The solution was concentrated, and the crude residue was directly purified by column chromatography (0→8% MeOH/DCM) to provide the desired product (85 mg, 0.140 mmol, 65% yield) as a yellow solid. LC/MS (ESI) m/z: [M+H]⁺ calcd for C₂₇H₃₄BrF₂N₆O₃ 607.1; found 607.3; ¹H NMR (500 MHz, CD₃OD) δ 7.73 (dd, J=9.0, 1.6 Hz, 1H), 7.48 (dd, J=9.0, 6.4 Hz, 1H), 5.30 (d, J=53.3 Hz, 1H), 4.73-4.63 (m, 1H), 4.47-4.32 (m, 2H), 4.29 (d, J=10.4 Hz, 1H), 4.23 (d, J=10.4 Hz, 1H), 4.08-3.98 (m, 1H), 3.67-3.59 (m, 1H), 3.56-3.43 (m, 2H), 3.27-3.14 (m, 4H), 3.06-2.84 (m, 3H), 2.37-2.11 (m, 3H), 2.03-1.87 (m, 3H), 1.51 (s, 9H).

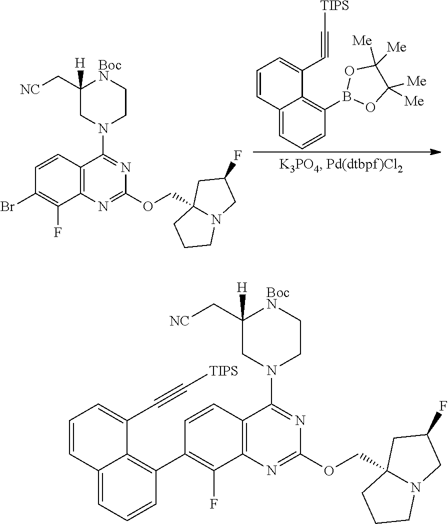

### Step 5: Preparation of tert-butyl (S)-2-(cyanomethyl)-4-(8-fluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazolin-4-yl)piperazine-1-carboxylate

tert-Butyl (S)-4-(7-bromo-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate (70 mg, 0.115 mmol), triisopropyl((8-(4,4,5,5-tetramethyl-1,3,2-dioxaborolan-2-yl)naphthalen-1-yl)ethynyl)silane (50 mg, 0.12 mmol), and [1,1′-bis(di-tert-butylphosphino)ferrocene]dichloropalladium (II) (3.8 mg, 5.8 mol) were combined as solids in a microwave vial. The vial was sealed. The atmosphere was evacuated and replaced with nitrogen. This process was performed three times. Degassed dioxane (5 mL) and potassium phosphate solution (2.0 M in water, 170 μL, 0.35 mmol) were added, and the reaction mixture was heated at 100° C. in the microwave for 2 h. The reaction mixture was directly concentrated, and the crude residue was purified by column chromatography (0→8% MeOH/DCM) to provide the desired product (190 mg, 0.024 mmol, 91% yield). LC/MS (ESI) m/z: [M+H]⁺ calcd for C₄₈H₆₁F₂N₆O₃Si 835.4. found 835.4; ¹H NMR (500 MHz, CD₃OD) δ 8.07-7.99 (m, 2H), 7.88-7.82 (m, 1H), 7.80-7.75 (m, 1H), 7.63-7.58 (m, 1H), 7.54-7.48 (m, 1H), 7.46-7.39 (m, 1H), 7.39-7.33 (m, 1H), 5.30 (d, J=53.3 Hz, 1H), 4.78-4.69 (m, 1H), 4.56-4.34 (m, 2H), 4.34-4.02 (m, 4H), 3.48-3.37 (m, 1H), 3.27-3.13 (m, 4H), 3.11-2.85 (m, 3H), 2.47-2.12 (m, 3H), 2.04-1.87 (m, 3H), 1.55-1.49 (m, 9H), 0.92-0.84 (m, 18H), 0.61-0.51 (m, 3H).

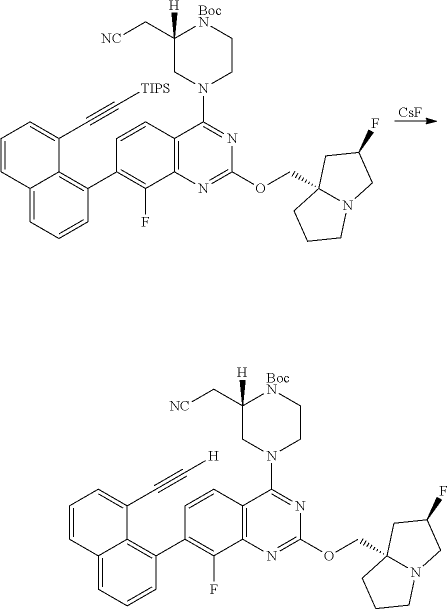

### Step 6: Preparation of tert-butyl (S)-2-(cyanomethyl)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)piperazine-1-carboxylate

tert-Butyl (S)-2-(cyanomethyl)-4-(8-fluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazolin-4-yl)piperazine-1-carboxylate (50 mg, 0.060 mmol) was dissolved in DMF (3 mL), and cesium fluoride (27 mg, 0.18 mmol) was added as a solid. The mixture was heated at 60° C. for 2 h. The suspension was cooled to room temperature and diluted with EtOAc (10 mL) and water (5 mL). The layers were separated, and the organic phase was further washed with brine (5 mL), dried over magnesium sulfate, filtered, and concentrated. The crude residue was used directly in the next step, without additional purification (quantitative yield assumed). LC/MS (ESI) m/z: [M+H]⁺ calcd for C₃₉H₄₁F₂N₆O₃ 679.3. found 679.3.

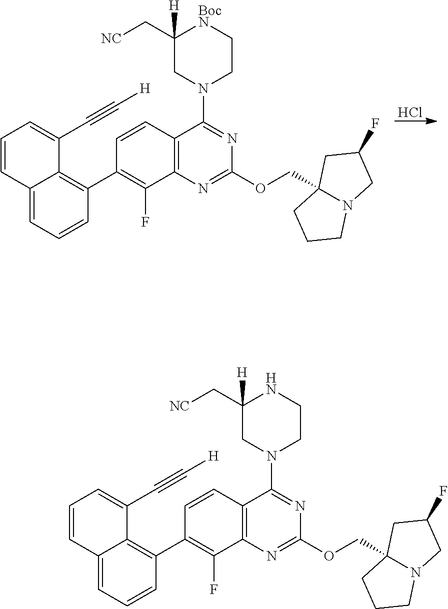 [SMILES: C#Cc1cccc2cccc(-c3ccc4c(N5CCN(C(=O)OC(C)(C)C)[C@@H](CC#N)C5)nc(OC[C@@]56CCCN5C[C@H](F)C6)nc4c3F)c12 ; Cl ; C#Cc1cccc2cccc(-c3ccc4c(N5CCN[C@@H](CC#N)C5)nc(OC[C@@]56CCCN5C[C@H](F)C6)nc4c3F)c12]

### Step 7: Preparation of 2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile

tert-butyl (S)-2-(cyanomethyl)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)piperazine-1-carboxylate (40 mg, 0.059 mmol) was dissolved in dioxane (1.0 mL), and a solution of HCL (4.0 M in dioxane, 1.0 mL, 4.0 mmol) was added dropwise. The reaction mixture was stirred at room temperature for 2 h. The solution was directly concentrated, and the crude residue was evaporated from DCM (3×5 mL) to provide the desired product as its HCl salt (quantitative yield assumed). LC/MS (ESI) m/z: [M+H]⁺ calcd for C₃₄H₃₃F₂N₆O 579.3. found 579.4.

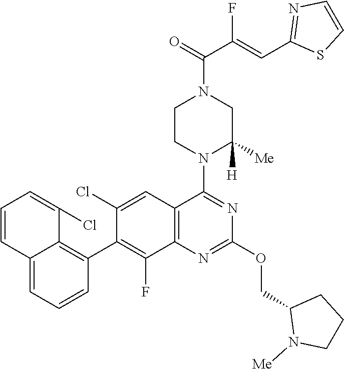 [SMILES: C[C@H]1CN(C(=O)/C(F)=C/c2nccs2)CCN1c1nc(OC[C@@H]2CCCN2C)nc2c(F)c(-c3cccc4cccc(Cl)c34)c(Cl)cc12]

### Example 1

### (Z)-1-((3S)-4-(6-chloro-7-(8-chloronaphthalen-1-yl)-8-fluoro-2-(((S)-1-methylpyrrolidin-2-yl)methoxy)quinazolin-4-yl)-3-methylpiperazin-1-yl)-2-fluoro-3-(thiazol-2-yl)prop-2-en-1-one

6-Chloro-7-(8-chloronaphthalen-1-yl)-8-fluoro-4-((S)-2-methylpiperazin-1-yl)-2-(((S)-1-methylpyrrolidin-2-yl)methoxy)quinazoline (Intermediate 1, 14 mg, 0.025 mmol) and (Z)-2-fluoro-3-(thiazol-2-yl)acrylic acid (Intermediate 25, 22 mg, 0.13 mmol) were combined as solids and dissolved in DMF (0.4 mL). 1-Methylimidazole (20 μL, 0.25 mmol) was added followed by chloro-N,N,N′,N′-tetramethylformamidinium hexafluorophosphate (35 mg, 0.13 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 0.1% TFA/28% 95:5 MeCN:H₂O with 0.1% TFA→100% 95:5 MeCN:H₂O with 0.1% TFA; λ=220 nm) to provide the desired product as a bis-TFA salt (8 mg, 0.009 mmol, 34% yield) as an off-white solid which is a mixture of two atropisomers. LC/MS (ESI) m/z: [M+H]⁺ calcd for C₃₅H₃₃C₁₂F₂N₆O₂S 709.2. found 709.0. NMR: Spectrum is a mixture of atropisomers; a water suppression method was used ¹H NMR (500 MHz, DMSO-d₆) δ 8.24-8.18 (m, 1H), 8.15-8.07 (m, 1H), 8.04-7.98 (m, 2H), 7.98-7.85 (m, 1H), 7.77-7.71 (m, 1H), 7.71-7.63 (m, 1H), 7.63-7.54 (m, 1H), 7.54-7.43 (m, 1H), 7.10 (d, J=37.6 Hz, 1H), 4.93-4.80 (m, 1H), 4.77-4.69 (m, 1H), 4.65-4.55 (m, 1H), 4.35-4.11 (m, 2H), 3.88-3.56 (m, 2H), 2.98-2.92 (m, 3H), 2.86-2.75 (m, 1H), 2.31-2.22 (m, 1H), 2.10-2.02 (m, 1H), 1.98-1.85 (m, 2H), 1.45-1.33 (m, 3H).

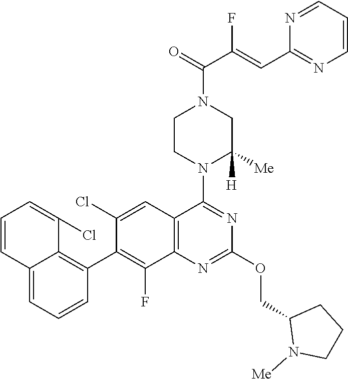 [SMILES: C[C@H]1CN(C(=O)/C(F)=C/c2ncccn2)CCN1c1nc(OC[C@@H]2CCCN2C)nc2c(F)c(-c3cccc4cccc(Cl)c34)c(Cl)cc12]

### Example 2

### (Z)-1-((3S)-4-(6-chloro-7-(8-chloronaphthalen-1-yl)-8-fluoro-2-(((S)-1-methylpyrrolidin-2-yl)methoxy)quinazolin-4-yl)-3-methylpiperazin-1-yl)-2-fluoro-3-(pyrimidin-2-yl)prop-2-en-1-one

6-Chloro-7-(8-chloronaphthalen-1-yl)-8-fluoro-4-((S)-2-methylpiperazin-1-yl)-2-(((S)-1-methylpyrrolidin-2-yl)methoxy)quinazoline (Intermediate 1, 14 mg, 0.025 mmol) and (Z)-2-fluoro-3-(pyrimidin-2-yl)acrylic acid (Intermediate 26, 22 mg, 0.13 mmol) were combined as solids and dissolved in DMF (0.4 mL). 1-Methylimidazole (20 μL, 0.25 mmol) was added followed by chloro-N,N,N′,N′-tetramethylformamidinium hexafluorophosphate (35 mg, 0.13 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 0.1% TFA/23% 95:5 MeCN:H₂O with 0.1% TFA→100% 95:5 MeCN:H₂O with 0.1% TFA; λ=220 nm) to provide the desired product as a bis-TFA salt as a mixture of two atropisomers (5.8 mg, 0.006 mmol, 25% yield). LC/MS (ESI) m/z: [M+H]⁺ calcd for C₃₆H₃₄C₁₂F₂N₇O₂ 704.2. found 704.1. NMR: Spectrum is a mixture of atropisomers; a water suppression method was used ¹H NMR (500 MHz, DMSO-d₆) δ 8.89 (d, J=4.9 Hz, 2H), 8.24-8.19 (m, 1H), 8.13-8.09 (m, 1H), 7.97-7.89 (m, 1H), 7.77-7.72 (m, 1H), 7.68-7.64 (m, 1H), 7.58-7.58 (m, 1H), 7.59-7.54 (m, 1H), 7.51-7.47 (m, 1H), 7.45 (t, J=4.9 Hz, 1H), 6.61 (d, J=35.2 Hz, 1H), 4.96-4.80 (m, 1H), 4.79-4.69 (m, 1H), 4.66-4.55 (m, 1H), 4.39-4.04 (m, 3H), 3.89-3.75 (m, 2H), 3.00-2.91 (m, 3H), 2.87-2.79 (m, 1H), 2.30-2.21 (m, 1H), 2.10-2.06 (m, 1H), 1.99-1.86 (m, 2H), 1.44-1.35 (m, 3H).

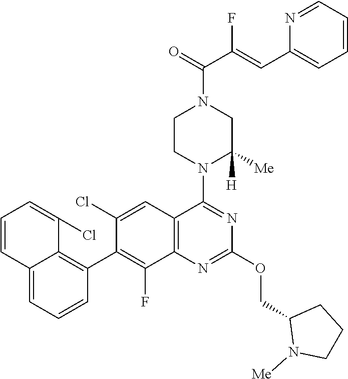 [SMILES: C[C@H]1CN(C(=O)/C(F)=C/c2ccccn2)CCN1c1nc(OC[C@@H]2CCCN2C)nc2c(F)c(-c3cccc4cccc(Cl)c34)c(Cl)cc12]

### Example 3

### (Z)-1-((3S)-4-(6-chloro-7-(8-chloronaphthalen-1-yl)-8-fluoro-2-(((S)-1-methylpyrrolidin-2-yl)methoxy)quinazolin-4-yl)-3-methylpiperazin-1-yl)-2-fluoro-3-(pyridin-2-yl)prop-2-en-1-one

6-Chloro-7-(8-chloronaphthalen-1-yl)-8-fluoro-4-((S)-2-methylpiperazin-1-yl)-2-(((S)-1-methylpyrrolidin-2-yl)methoxy)quinazoline (Intermediate 1, 14 mg, 0.025 mmol) and (Z)-2-fluoro-3-(pyridin-2-yl)acrylic acid (Intermediate 27, 22 mg, 0.13 mmol) were combined as solids and dissolved in DMF (0.4 mL). 1-Methylimidazole (20 μL, 0.25 mmol) was added followed by chloro-N,N,N′,N′-tetramethylformamidinium hexafluorophosphate (35 mg, 0.13 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 81% 5:95 MeCN:H₂O with 0.1% TFA/19% 95:5 MeCN:H₂O with 0.1% TFA→100% 95:5 MeCN:H₂O with 0.1% TFA; λ=220 nm) to provide the desired product as a mixture of atropisomers. A second purification using chiral SFC (column: Chiralcel OD-H, 30 mm×250 mm, 5 m particles; flow rate: 100 mL/min; column temperature: 40° C., isocratic: 80% CO₂:20% MeOH with 0.1% NH₄OH; λ=254 nm) provided the desired compound, a single atropisomer of unknown absolute stereochemistry, as the bis-TFA salt (1.3 mg, 0.001 mmol, 6% yield). LC/MS (ESI) m/z: [M+H]⁺ calcd for C₃₇H₃₅Cl₂F₂N₆O₂ 702.9; found 702.9.

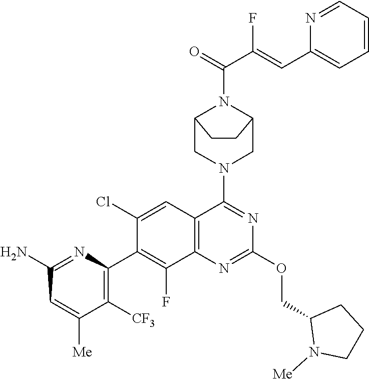

### Example 4

### (Z)-1-((1R,5S)-3-((7M)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one

(5M)-6-(4-((1R,5S)-3,8-diazabicyclo[3.2.1]octan-3-yl)-6-chloro-8-fluoro-2-(((S)-1-methylpyrrolidin-2-yl)methoxy)quinazolin-7-yl)-4-methyl-5-(trifluoromethyl)pyridin-2-amine (Intermediate 6, 3 mg, 0.005 mmol) and (Z)-2-fluoro-3-(pyridin-2-yl)acrylic acid (Intermediate 27, 4 mg, 0.026 mmol) were combined as solids and dissolved in DMF (0.3 mL). 1-Methylimidazole (4 μL, 0.05 mmol) was added followed by chloro-N,N,N′,N′-tetramethylformamidinium hexafluorophosphate (7.3 mg, 0.026 mmol) The reaction mixture was stirred at room temperature for 5 min. The reaction mixture was directly purified by reverse phase HPLC (column: Xbridge C18, 19 mm×200 mm, 5 μm particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product as a single atromisomer (2.7 mg, 0.004 mmol, 72% yield). LC/MS (ESI) m/z: [M+H]⁺ calcd for C₃₅H₃₅ClF₅N₈O₂ 729.2; found 729.1. NMR: a water suppression method was used ¹H NMR (500 MHz, DMSO-d₆) δ 8.66 (d, J=5.0 Hz, 1H), 7.90 (dd, J=8.0, 7.0 Hz, 1H), 7.87 (s, 1H), 7.81 (d, J=8.0 Hz, 1H), 7.39 (dd, J=7.0, 5.0 Hz, 1H), 6.83 (br s, 2H), 6.81 (d, J=37.8 Hz, 1H), 6.49 (s, 1H), 4.82-4.73 (m, 2H), 4.61-4.51 (m, 1H), 4.45-4.36 (m, 2H), 4.29-4.16 (m, 1H), 3.87-3.56 (m, 1H), 3.44-3.35 (m, 1H), 2.47-2.24 (m, 6H), 2.04-1.82 (m, 4H), 1.81-1.63 (m, 4H).

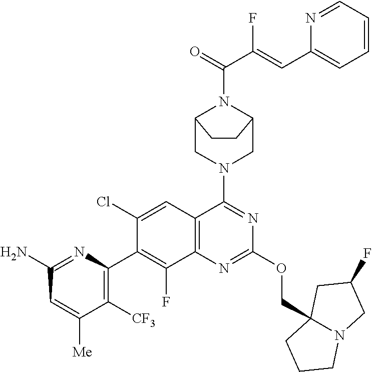

### Example 5

### (Z)-1-((1R,5S)-3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,8-diazabicyclo[3.2.1]octan-8-yl)-2-fluoro-3-(pyridin-2-yl)prop-2-en-1-one

(5M)-6-(4-((1R,5S)-3,8-diazabicyclo[3.2.1]octan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine (Intermediate 9, 3 mg, 0.005 mmol) and (Z)-2-fluoro-3-(pyridin-2-yl)acrylic acid (Intermediate 27, 4 mg, 0.026 mmol) were combined as solids and dissolved in DMF (0.3 mL). 1-Methylimidazole (4 μL, 0.05 mmol) was added followed by chloro-N,N,N′,N′-tetramethylformamidinium hexafluorophosphate (7 mg, 0.026 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product as a single atropisomer (3.5 mg, 0.005 mmol, 94% yield). LC/MS (ESI) m/z: [M+H]⁺ calcd for C₃₇H₃₆ClF₆N₈O₂ 773.3. found 773.1. NMR: a water suppression method was used; ¹H NMR (500 MHz, DMSO-d₆) δ 8.66 (d, J=5.0 Hz, 1H), 7.90 (dd, J=8.0, 7.0 Hz, 1H), 7.89 (s, 1H), 7.81 (d, J=8.0 Hz, 1H), 7.39 (dd, J=7.0, 5.0 Hz, 1H), 6.84 (br s, 1H), 6.82 (d, J=37.9 Hz, 1H), 6.50 (s, 1H), 5.56-5.33 (m, 1H), 4.85-4.73 (m, 2H), 4.66-4.50 (m, 1H), 4.47-4.28 (m, 1H), 3.86-3.54 (m, 1H), 2.40-2.34 (m, 3H), 2.23-1.71 (m, 8H).

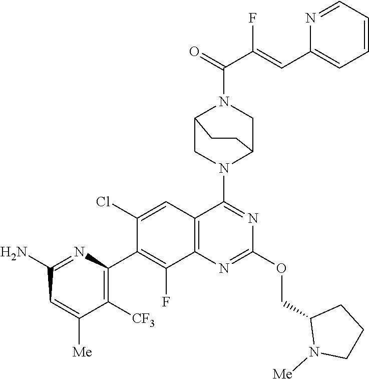

### Example 6

### (Z)-1-(5-((7M)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-2,5-diazabicyclo[2.2.2]octan-2-yl)-2-fluoro-3-(pyridin-2-yl)prop-2-en-1-one

(5M)-6-(4-(2,5-Diazabicyclo[2.2.2]octan-2-yl)-6-chloro-8-fluoro-2-(((S)-1-methylpyrrolidin-2-yl)methoxy)quinazolin-7-yl)-4-methyl-5-(trifluoromethyl)pyridin-2-amine (Intermediate 7, 3 mg, 0.005 mmol) and (Z)-2-fluoro-3-(pyridin-2-yl)acrylic acid (Intermediate 27, 4 mg, 0.026 mmol) were combined as solids and dissolved in DMF (0.3 mL). 1-Methylimidazole (4 μL, 0.05 mmol) was added followed by chloro-N,N,N′,N′-tetramethylformamidinium hexafluorophosphate (7.3 mg, 0.026 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (1.1 mg, 0.001 mmol, 29% yield). LC/MS (ESI) m/z: [M+H]⁺ calcd for C₃₅H₃₅ClF₅N₈O₂ 729.2. found 729.1. NMR: spectrum is complicated by presence of diastereomers (C4 amine); a water suppression method was used ¹H NMR (500 MHz, DMSO-d₆) δ 8.66-8.62 (m, 1H), 8.09-8.01 (m, 1H), 7.91-7.85 (m, 1H), 7.78 (br d, J=8.0 Hz, 1H), 7.38 (br d, J=4.4 Hz, 1H), 6.85 (br s, 2H), 6.61 (d, J=39.0 Hz, 1H), 6.49 (s, 1H), 5.13-4.98 (m, 1H), 4.69-4.43 (m, 3H), 4.26-4.15 (m, 1H), 4.09-4.01 (m, 1H), 3.88-3.81 (m, 1H), 3.74-3.65 (m, 1H), 2.40-2.32 (m, 3H), 2.29-2.03 (m, 3H), 1.99-1.92 (m, 2H), 1.89-1.70 (m, 3H).

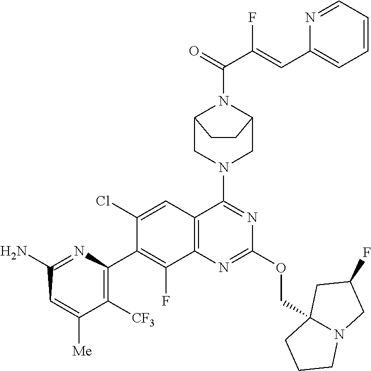

### Example 7

### (Z)-1-((1R,5S)-3-((7M)-7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octan-8-yl)-2-fluoro-3-(pyridin-2-yl)prop-2-en-1-one

(5M)-6-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-methyl-5-(trifluoromethyl)pyridin-2-amine (Intermediate 10, 3 mg, 0.005 mmol) and (Z)-2-fluoro-3-(pyridin-2-yl)acrylic acid (Intermediate 27, 4 mg, 0.026 mmol) were combined as solids and dissolved in DMF (0.3 mL). 1-Methylimidazole (4 μL, 0.055 mmol) was added followed by chloro-N,N,N′,N′-tetramethylformamidinium hexafluorophosphate (7 mg, 0.026 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to provide the desired product (1.4 mg, 0.002 mmol, 38% yield). LC/MS (ESI) m/z: [M+H]+ calcd for C₃₇H₃₆ClF₆N₈O₂ 773.2. found 773.2. NMR: a water suppression method was used ¹H NMR (500 MHz, DMSO-d₆) δ 8.66 (d, J=5.3 Hz, 1H), 7.91 (dd, J=7.8, 7.0 Hz, 1H), 7.91 (br s, 1H), 7.81 (d, J=7.9 Hz, 1H), 7.39 (dd, J=7.1, 5.3 Hz, 1H), 6.83 (br s, 2H), 6.82 (d, J=37.9 Hz, 1H), 6.50 (s, 1H), 5.63-5.43 (m, 1H), 4.85-4.75 (m, 2H), 4.71-4.57 (m, 1H), 4.55-4.35 (m, 2H), 3.87-3.53 (m, 1H), 2.48-2.41 (m, 2H), 2.40-2.34 (m, 3H), 2.33-2.23 (m, 1H), 2.22-2.06 (m, 2H), 2.05-1.81 (m, 4H), 1.79-1.69 (m, 1H).

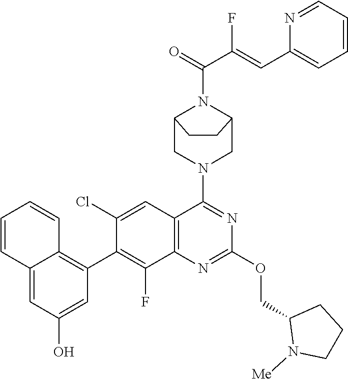 [SMILES: CN1CCC[C@H]1COc1nc(N2CC3CCC(C2)N3C(=O)/C(F)=C/c2ccccn2)c2cc(Cl)c(-c3cc(O)cc4ccccc34)c(F)c2n1]

### Example 8

### (Z)-1-((1R,5S)-3-(6-chloro-8-fluoro-7-(3-hydroxynaphthalen-1-yl)-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one

5-(4-((1R,5S)-3,8-Diazabicyclo[3.2.1]octan-3-yl)-6-chloro-8-fluoro-2-(((S)-1-methylpyrrolidin-2-yl)methoxy)quinazolin-7-yl)naphthalen-2-ol (Intermediate 8, 3 mg, 0.005 mmol) and (Z)-2-fluoro-3-(pyridin-2-yl)acrylic acid (Intermediate 27, 4.6 mg, 0.027 mmol) were combined as solids and dissolved in DMF (0.3 mL). 1-Methylimidazole (4 μL, 0.25 mmol) was added followed by chloro-N,N,N′,N′-tetramethylformamidinium hexafluorophosphate (7.7 mg, 0.026 mmol). The reaction mixture was stirred at room temperature for 5 min. A saturated aqueous solution of ammonium hydroxide (100 μL) was added and the reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product as a mixture of two atropisomers (2.4 mg, 0.003 mmol, 62% yield). LC/MS (ESI) m/z: [M+H]⁺ calcd for C₃₈H₃₆ClF₂N₆O₃ 697.2. found 697.1. NMR: spectrum is complicated by presence of diastereomers; a water suppression method was used ¹H NMR (500 MHz, DMSO-d₆) δ 10.02 (s, 1H), 8.66 (br d, J=4.7 Hz, 1H), 8.00 (s, 1H), 7.94-7.86 (m, 1H), 7.86-7.77 (m, 2H), 7.47-7.37 (m, 2H), 7.29 (d, J=2.3 Hz, 1H), 7.25-7.19 (m, 2H), 7.06 (d, J=2.3 Hz, 1H), 6.83 (d, J=38.0 Hz, 1H), 4.85-4.76 (m, 2H), 4.62-4.51 (m, 2H), 4.48-4.40 (m, 1H), 4.37-4.25 (m, 1H), 3.84-3.66 (m, 1H), 3.44-3.31 (m, 1H), 2.09-1.92 (m, 2H), 1.89-1.80 (m, 3H), 1.78-1.62 (m, 3H)

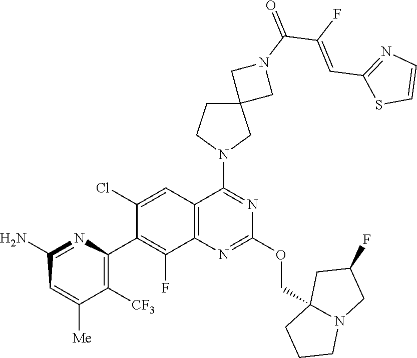

### Example 9

### (Z)-1-(6-((7M)-7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-2,6-diazaspiro[3.4]octan-2-yl)-2-fluoro-3-(thiazol-2-yl)prop-2-en-1-one

(5M)-6-(6-Chloro-8-fluoro-2-(((2R,7aS)-2-fluorotetrahydro-1H-pyrrolizin-7a(5H)-yl)methoxy)-4-(2,6-diazaspiro[3.4]octan-6-yl)quinazolin-7-yl)-4-methyl-5-(trifluoromethyl)pyridin-2-amine (Intermediate 24-1, 3 mg, 0.005 mmol) and (Z)-2-fluoro-3-(thiazol-2-yl)acrylic acid (Intermediate 25, 2.5 mg, 0.014 mmol) were combined as solids and dissolved in DMF (0.3 mL). 1-Methylimidazole (2 μL, 0.029 mmol) was added followed by chloro-N,N,N′,N′-tetramethylformamidinium hexafluorophosphate (4 mg, 0.014 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (1.7 mg, 0.002 mmol, 45% yield). LC/MS (ESI) m/z: [M+H]⁺ calcd for C₃₅H₃₄ClF₆N₈O₂S 779.2. found 779.0. NMR: ¹H NMR (500 MHz, DMSO-d₆) δ 8.09 (s, 1H), 8.01-7.92 (m, 2H), 7.15 (d, J=36.3 Hz, 1H), 6.76 (br s, 2H), 6.51 (s, 1H), 5.50-5.31 (m, 1H), 4.62-4.55 (m, 1H), 4.52-4.43 (m, 1H), 4.37-4.28 (m, 1H), 4.26-4.18 (m, 1H), 4.17-4.12 (m, 1H), 4.07-3.99 (m, 1H), 3.09-2.98 (m, 1H), 2.35 (br d, J=1.2 Hz, 3H), 2.33-2.21 (m, 4H), 2.17-2.07 (m, 1H), 2.04-1.83 (m, 3H)

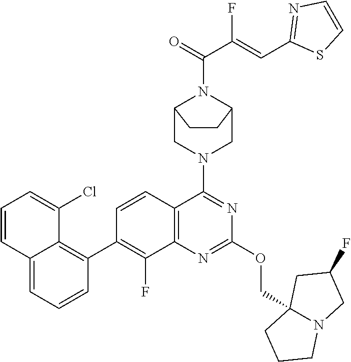 [SMILES: O=C(/C(F)=C/c1nccs1)N1C2CCC1CN(c1nc(OC[C@@]34CCCN3C[C@H](F)C4)nc3c(F)c(-c4cccc5cccc(Cl)c45)ccc13)C2]

### Example 10

### (Z)-1-((1R,5S)-3-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octan-8-yl)-2-fluoro-3-(thiazol-2-yl)prop-2-en-1-one

4-((1R,5S)-3,8-Diazabicyclo[3.2.1]octan-3-yl)-7-(8-chloronaphthalen-1-yl)-8-fluoro-2-(((2R,7aS)-2-fluorotetrahydro-1H-pyrrolizin-7a(5H)-yl)methoxy)quinazoline (Intermediate 16, 3 mg, 0.005 mmol) and (Z)-2-fluoro-3-(thiazol-2-yl)acrylic acid (Intermediate 25, 2.5 mg, 0.016 mmol) were combined as solids and dissolved in DMF (0.3 mL). 1-Methylimidazole (2.5 μL, 0.031 mmol) was added followed by chloro-N,N,N′,N′-tetramethylformamidinium hexafluorophosphate (4 mg, 0.016 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 66% 5:95 MeCN:H₂O with 10 mM AA/34% 95:5 MeCN:H₂O with 10 mM AA→100% 95:5 MeCN:H₂O with 10 mM AA; λ=220 nm) to provide the desired product (1.7 mg, 0.002 mmol, 44% yield). LC/MS (ESI) m/z: [M+H]⁺ calcd for C₃₈H₃₅ClF₃N₆O₂S 731.2; found 730.9. NMR: this spectrum complicated by presence of atropisomers that do not convert on the NAMR timescale (but do not separate on chiral chromatography). ¹H NMR (500 MHz, DMSO-d₆) δ 8.16-8.12 (m, 1H), 8.09-8.04 (m, 1H), 8.02-7.99 (m, 1H), 7.99-7.95 (m, 1H), 7.85-7.80 (m, 1H), 7.71-7.66 (m, 1H), 7.64-7.60 (m, 1H), 7.58-7.52 (m, 1H), 7.51-7.48 (m, 1H), 7.29-7.23 (m, 1H), 7.19 (d, J=36.6 Hz, 1H), 5.35-5.18 (m, 1H), 4.83-4.74 (m, 2H), 4.51 (br d, J=12.9 Hz, 1H), 4.44 (br d, J=12.9 Hz, 1H), 4.11 (dd, J=10.3, 2.0 Hz, 1H), 4.02 (d, J=10.3 Hz, 1H), 3.13-2.99 (m, 3H), 2.85-2.76 (m, 1H), 2.19-1.90 (m, 5H), 1.89-1.67 (m, 5H)

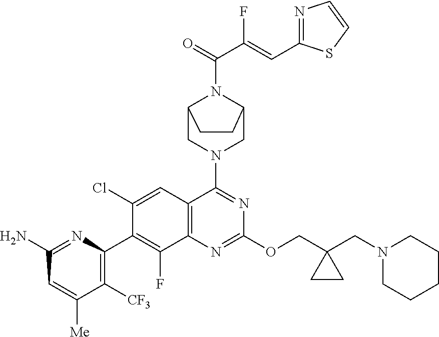

### Example 11

### (Z)-1-((1R,5S)-3-((7M)-7-(6-amino-4-methyl-3-(trifluoromethyl)pyridin-2-yl)-6-chloro-8-fluoro-2-((1-(piperidin-1-ylmethyl)cyclopropyl)methoxy)quinazolin-4-yl)-3,8-diazabicyclo[3.2.1]octan-8-yl)-2-fluoro-3-(thiazol-2-yl)prop-2-en-1-one

(5M)-6-(4-((1R,5S)-3,8-diazabicyclo[3.2.1]octan-3-yl)-6-chloro-8-fluoro-2-((1-(piperidin-1-ylmethyl)cyclopropyl)methoxy)quinazolin-7-yl)-4-methyl-5-(trifluoromethyl)pyridin-2-amine (Intermediate 12, 3 mg, 0.005 mmol) and (Z)-2-fluoro-3-(thiazol-2-yl)acrylic acid (Intermediate 25, 2.5 mg, 0.014 mmol) were combined as solids and dissolved in DMF (0.3 mL). 1-Methylimidazole (2 μL, 0.028 mmol) was added followed by chloro-N,N,N′,N′-tetramethylformamidinium hexafluorophosphate (4 mg, 0.014 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (3.5 mg, 0.004 mmol, 93% yield). LC/MS (ESI) m/z: [M+H]⁺ calcd for C₃₇H₃₉ClF₅N₈O₂S 731.2. found 730.9. NMR: ¹H NMR (500 MHz, DMSO-d₆) δ 8.02-7.99 (m, 1H), 7.98-7.94 (m, 1H), 7.83 (s, 1H), 7.18 (d, J=36.7 Hz, 1H), 6.76 (br s, 2H), 6.51 (s, 1H), 4.76 (br d, J=10.8 Hz, 2H), 4.60-4.46 (m, 1H), 4.41-4.31 (m, 1H), 4.29-4.20 (m, 2H), 2.45-2.29 (m, 8H), 2.02-1.90 (m, 1H), 1.89-1.82 (m, 2H), 1.78-1.70 (m, 1H), 1.48-1.37 (m, 4H), 1.36-1.27 (m, 2H), 0.62 (br s, 2H), 0.41 (br s, 2H)

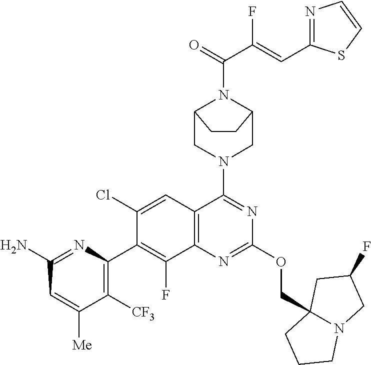

### Example 12

### (Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.1.1]heptan-6-yl)-2-fluoro-3-(thiazol-2-yl)prop-2-en-1-one

(5M)-6-(4-(3,6-Diazabicyclo[3.1.1]heptan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine (Intermediate 15, 3 mg, 0.005 mmol) and (Z)-2-fluoro-3-(thiazol-2-yl)acrylic acid (Intermediate 25, 2.6 mg, 0.015 mmol) were combined as solids and dissolved in DMF (0.3 mL). 1-Methylimidazole (2 μL, 0.030 mmol) was added followed by chloro-N,N,N′,N′-tetramethylformamidinium hexafluorophosphate (4 mg, 0.015 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (1.7 mg, 0.002 mmol, 45% yield). LC/MS (ESI) m/z: [M+H]⁺ calcd for C₃₄H₃₂ClF₆N₈O₂S 765.2. found 764.9. NMR: ¹H NMR (500 MHz, DMSO-d₆) δ 8.27 (s, 1H), 8.01-7.98 (m, 1H), 7.98-7.95 (m, 1H), 7.16 (d, J=36.2 Hz, 1H), 6.76 (br s, 2H), 6.50 (s, 1H), 5.44-5.28 (m, 1H), 5.11-4.99 (m, 1H), 4.61 (br d, J=1.7 Hz, 1H), 4.55 (s, 5H), 2.88-2.79 (m, 1H), 2.38-2.31 (m, 4H), 2.05-1.84 (m, 3H), 1.79-1.71 (m, 2H)

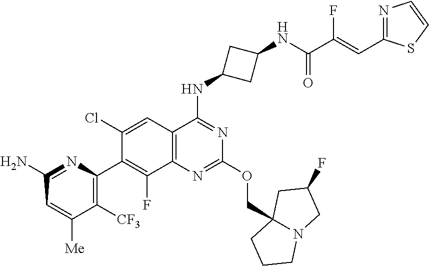

### Example 13

### (Z)-N-((1S,3s)-3-(((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)amino)cyclobutyl)-2-fluoro-3-(thiazol-2-yl)acrylamide

(1s,3S)-N₁-((7M)-7-(6-Amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)cyclobutane-1,3-diamine (Intermediate 23, 3 mg, 0.005 mmol) and (Z)-2-fluoro-3-(thiazol-2-yl)acrylic acid (Intermediate 25, 2.6 mg, 0.015 mmol) were combined as solids and dissolved in DMF (0.3 mL). 1-Methylimidazole (4 μL, 0.050 mmol) was added followed by chloro-N,N,N′,N′-tetramethylformamidinium hexafluorophosphate (4 mg, 0.015 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 5:95 MeCN:H₂O with 10 mM AA→95:5 MeCN:H₂O with 10 mM AA; λ=220 nm) to provide the desired product (2.3 mg, 0.003 mmol, 61% yield). LC/MS (ESI) m/z: [M+H]⁺ calcd for C₃₃H₃₂ClF₆N₈O₂S 753.2. found 752.9. NMR: ¹H NMR (500 MHz, DMSO-d₆) δ 9.13 (d, J=6.7 Hz, 1H), 8.67 (br d, J=4.7 Hz, 1H), 8.36 (s, 1H), 8.03-8.00 (m, 1H), 8.00-7.97 (m, 1H), 7.23 (d, J=36.0 Hz, 1H), 6.84 (s, 2H), 6.49 (s, 1H), 5.44-5.28 (m, 1H), 4.38-4.29 (m, 1H), 4.19-4.09 (m, 2H), 4.08-4.00 (m, 1H), 3.02-2.91 (m, 1H), 2.83-2.73 (m, 3H), 2.39-2.34 (m, 3H), 2.33-2.20 (m, 3H), 1.95-1.89 (m, 2H), 1.89-1.80 (m, 2H), 1.75-1.63 (m, 1H).

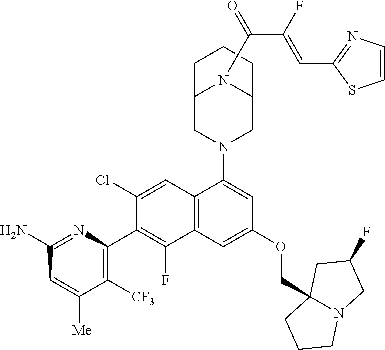

### Example 14

### (Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,9-diazabicyclo[3.3.1]nonan-9-yl)-2-fluoro-3-(thiazol-2-yl)prop-2-en-1-one

(5M)-6-(4-(3,9-Diazabicyclo[3.3.1]nonan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine (Intermediate 14, 3 mg, 0.005 mmol) and (Z)-2-fluoro-3-(thiazol-2-yl)acrylic acid (Intermediate 25, 2.4 mg, 0.014 mmol). 1-Methylimidazole (4 μL, 0.047 mmol) was added followed by chloro-N,N,N′,N′-tetramethylformamidinium hexafluorophosphate (4 mg, 0.014 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 5:95 MeCN:H₂O with 10 mM AA→95:5 MeCN:H₂O with 10 mM AA; λ=220 nm) to provide the desired product (2.1 mg, 0.003 mmol, 56% yield). LC/MS (ESI) m/z: [M+H]⁺ calcd for C₃₆H₃₆ClF₆N₈O₂S 793.2. found 793.0. NMR: ¹H NMR (500 MHz, DMSO-d₆) δ 8.02-7.96 (m, 2H), 7.86 (s, 1H), 7.07 (d, J=38.0 Hz, 1H), 6.83 (br s, 2H), 6.50 (s, 1H), 5.45-5.27 (m, 1H), 4.72-4.36 (m, 4H), 4.20-4.14 (m, 1H), 4.14-4.08 (m, 1H), 3.87-3.52 (m, 2H), 3.00-2.76 (m, 1H), 2.63-2.53 (m, 1H), 2.37 (br s, 3H), 2.35-2.26 (m, 1H), 2.10-1.78 (m, 9H), 1.72-1.63 (m, 1H), 1.61-1.52 (m, 1H).

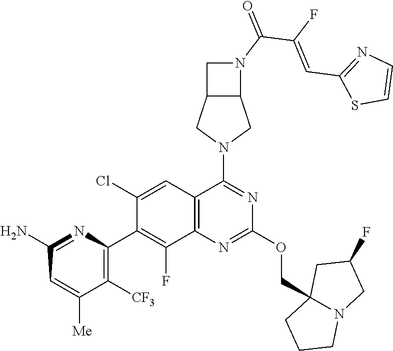

### Example 15

### (Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.2.0]heptan-6-yl)-2-fluoro-3-(thiazol-2-yl)prop-2-en-1-one

(5M)-6-(4-(3,6-Diazabicyclo[3.2.0]heptan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine (Intermediate 24, 3 mg, 0.005 mmol) (as a mixture of two cis-diastereomers) and (Z)-2-fluoro-3-(thiazol-2-yl)acrylic acid (Intermediate 25, 2.5 mg, 0.015 mmol) were combined as solids and dissolved in DMF (0.3 mL) 1-Methylimidazole (4 μL, 0.049 mmol) was added followed by chloro-N,N,N′,N′-tetramethylformamidinium hexafluorophosphate (4 mg, 0.015 mmol) The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 87% 5:95 MeCN:H₂O with 0.1% TFA/13% 95:5 MeCN:H₂O with 0.1% TFA→100% 95:5 MeCN:H₂O with 0.1% TFA; λ=220 nm) to provide two products as single diastereomers and as bis-TFA salts, but of unknown absolute stereochemistry: Isomer 1 (1.3 mg, 0.001 mmol, 27% yield) and Isomer 2 (1.2 mg, 0.001 mmol, 25% yield). Isomer 1): LC/MS (ESI) m/z: [M+H]⁺ calcd for C₃₄H₃₂ClF₆N₈O₂S 765.2. found 764.9. NMR: ¹H NMR (500 MHz, DMSO-d₆) δ 8.17-8.10 (m, 1H), 8.05-7.95 (m, 2H), 7.25 (s, 2H), 7.13 (d, J=35.9 Hz, 1H), 6.50 (s, 1H), 5.57-5.30 (m, 1H), 5.13-4.90 (m, 1H), 4.77-4.47 (m, 4H), 4.24-3.97 (m, 1H), 3.88-3.61 (m, 2H), 2.39-2.30 (m, 4H), 2.27-1.91 (m, 5H). Isomer 2: LC/MS (ESI) m/z: [M+H]⁺ calcd for C₃₄H₃₂ClF₆N₈O₂S 765.2. found 764.9. NMR: ¹H NMR (500 MHz, DMSO-d₆) δ 8.17-8.12 (m, 1H), 8.05-7.94 (m, 2H), 7.29-7.19 (m, 2H), 7.10 (d, J=37.6 Hz, 1H), 6.50 (s, 1H), 5.56-5.40 (m, 1H), 5.11-5.04 (m, 1H), 4.79-4.49 (m, 4H), 4.25-4.14 (m, 1H), 3.97-3.88 (m, 2H), 3.55-3.48 (m, 1H), 2.39-2.33 (m, 3H), 2.33-2.26 (m, 1H), 2.25-1.93 (m, 5H).

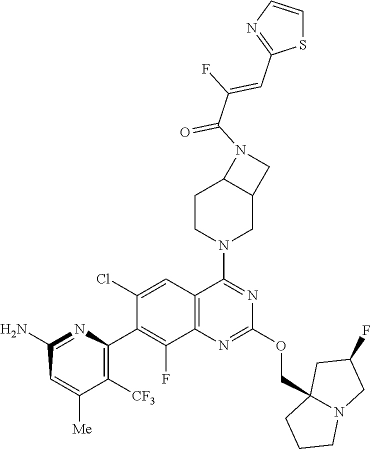

### Example 16

### (Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,7-diazabicyclo[4.2.0]octan-7-yl)-2-fluoro-3-(thiazol-2-yl)prop-2-en-1-one

(5M)-6-((7R)-4-(3,7-Diazabicyclo[4.2.0]octan-3-yl)-6-chloro-8-fluoro-2-(((2R,7aR)-2-fluorotetrahydro-1H-pyrrolizin-7a(5H)-yl)methoxy)quinazolin-7-yl)-4-methyl-5-(trifluoromethyl)pyridin-2-amine (Intermediate 22, 3 mg, 0.005 mmol) (as a mixture of two cis-diastereomers) and (Z)-2-fluoro-3-(thiazol-2-yl)acrylic acid (Intermediate 25, 2.5 mg, 0.014 mmol) were combined as solids and dissolved in DMF (0.3 mL). 1-Methylimidazole (4 μL, 0.049 mmol) was added followed by chloro-N,N,N′,N′-tetramethylformamidinium hexafluorophosphate (4 mg, 0.014 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (1.1 mg, 0.001 mmol, 29% yield) as a mixture of cis-diastereomers. LC/MS (ESI) m/z: [M+H]⁺ calcd for C₃₅H₃₃ClF₆N₈O₂S 779.2. found 778.9. NMR: compound is a mixture of two diastereomers; spectrum reported as obtained ¹H NMR (500 MHz, DMSO-d₆) δ 8.18-8.08 (m, 1H), 8.03-7.94 (m, 2H), 7.20 (d, J=35.8 Hz, 0.4H), 7.16 (d, J=36.0 Hz, 0.6H), 6.82 (br s, 2H), 6.49 (s, 1H), 5.42-5.24 (m, 1H), 5.14-5.06 (m, 0.4H), 4.79-4.70 (m, 0.6H), 4.61-4.52 (m, 0.6H), 4.45-4.01 (m, 4.4H), 3.96-3.82 (m, 2H), 3.32-3.20 (m, 0.6H), 3.00-2.91 (m, 0.4), 2.36 (br s, 3H), 2.33-2.26 (m, 2H), 2.20-2.08 (m, 1H), 1.96-1.76 (m, 4H), 1.72-1.61 (m, 1H).

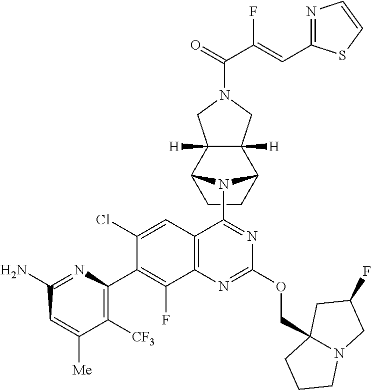

### Example 17

### (Z)-1-((3aR,4S,7R,7aS)-8-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-2H-4,7-epiminoisoindol-2-yl)-2-fluoro-3-(thiazol-2-yl)prop-2-en-1-one

(5M)-6-(6-Chloro-8-fluoro-2-(((2R,7aR)-2-fluorotetrahydro-1H-pyrrolizin-7a(5H)-yl)methoxy)-4-((3aR,4S,7R,7aS)-octahydro-1H-4,7-epiminoisoindol-8-yl)quinazolin-7-yl)-4-methyl-5-(trifluoromethyl)pyridin-2-amine (Intermediate 21, 3 mg, 0.005 mmol) and (Z)-2-fluoro-3-(thiazol-2-yl)acrylic acid (Intermediate 25, 2.4 mg, 0.014 mmol) were combined as solids and dissolved in DMF (0.3 mL). 1-Methylimidazole (4 μL, 0.048 mmol) was added followed by chloro-N,N,N′,N′-tetramethylformamidinium hexafluorophosphate (4 mg, 0.014 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (1.8 mg, 0.002 mmol, 48% yield). LC/MS (ESI) m/z: [M+H]⁺ calcd for C₃₇H₃₆ClF₆N₈O₂S 805.2. found 804.9. NMR: ¹H NMR (500 MHz, DMSO-d₆) δ 8.03-7.97 (m, 2H), 7.96 (s, 1H), 7.14 (d, J=36.9 Hz, 1H), 6.83 (br s, 2H), 6.50 (s, 1H), 5.46-5.29 (m, 1H), 4.89-4.83 (m, 2H), 4.26-3.91 (m, 3H), 3.15-2.95 (m, 1H), 2.36 (br s, 3H), 2.35-2.27 (m, 1H), 2.01-1.91 (m, 1H), 1.90-1.81 (m, 2H), 1.81-1.64 (m, 3H).

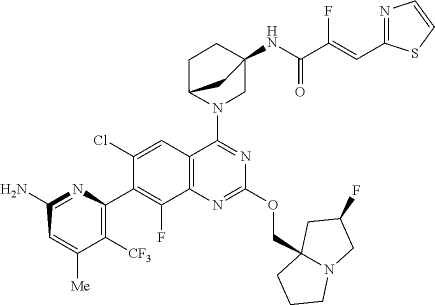

### Example 18

### (Z)-N-((1R,4R)-2-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-2-azabicyclo[2.2.1]heptan-4-yl)-2-fluoro-3-(thiazol-2-yl)acrylamide

(1R,4R)-2-((7M)-7-(6-Amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-2-azabicyclo[2.2.1]heptan-4-amine (Intermediate 18, 3 mg, 0.005 mmol) and (Z)-2-fluoro-3-(thiazol-2-yl)acrylic acid (Intermediate 25, 2.4 mg, 0.014 mmol) were combined as solids and dissolved in DMF (0.3 mL). 1-Methylimidazole (4 μL, 0.048 mmol) was added followed by chloro-N,N,N′,N′-tetramethylformamidinium hexafluorophosphate (4 mg, 0.014 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (1.6 mg, 0.002 mmol, 43% yield). LC/MS (ESI) m/z: [M+H]⁺ calcd for C₃₅H₃₄ClF₆N₈O₂S 779.2. found 778.9. NMR: ¹H NMR (500 MHz, DMSO-d₆) δ 9.33 (s, 1H), 8.04-7.97 (m, 2H), 7.91 (br s, 1H), 7.28 (d, J=35.9 Hz, 1H), 6.82 (br s, 2H), 6.50 (s, 1H), 5.43-5.27 (m, 1H), 4.90 (s, 1H), 4.11 (d, J=10.4 Hz, 1H), 4.05 (d, J=10.4 Hz, 1H), 2.98-2.91 (m, 1H), 2.89-2.75 (m, 1H), 2.59-2.52 (m, 1H), 2.36 (br s, 3H), 2.34-2.26 (m, 1H), 2.16 (br d, J=9.3 Hz, 1H), 2.09 (br d, J=9.3 Hz, 1H), 2.03-1.87 (m, 5H), 1.85-1.78 (m, 2H), 1.70-1.62 (m, 1H).

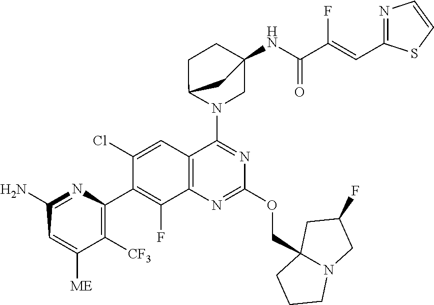

### Example 19

### (Z)-1-((2S,5R)-4-((7M)-7-(6-amino-4-methyl-3-(trifluoromethyl)pyridin-2-yl)-6-chloro-8-fluoro-2-((2-fluorotetrahydro-1H-pyrrolizin-7a(5H)-yl)methoxy)quinazolin-4-yl)-2,5-dimethylpiperazin-1-yl)-2-fluoro-3-(thiazol-2-yl)prop-2-en-1-one

The desired product was prepared from Intermediate 17 and Intermediate 25, using methods similar to those described herein, using the appropriate starting materials. ¹H NMR (500 MHz, DMSO-d₆) δ 8.01-7.99 (m, 1H), 7.99-7.95 (m, 1H), 7.83 (s, 1H), 7.06 (br d, J=39.6 Hz, 1H), 6.82 (br s, 2H), 6.50 (s, 1H), 5.39-5.18 (m, 1H), 4.85-4.69 (m, 1H), 4.68-4.34 (m, 1H), 4.20-4.00 (m, 3H), 3.87-3.73 (m, 1H), 3.14-3.00 (m, 1H), 2.90-2.76 (m, 1H), 2.36 (br s, 3H), 2.21-1.97 (m, 3H), 1.88-1.71 (m, 3H), 1.43-1.19 (m, 6H).

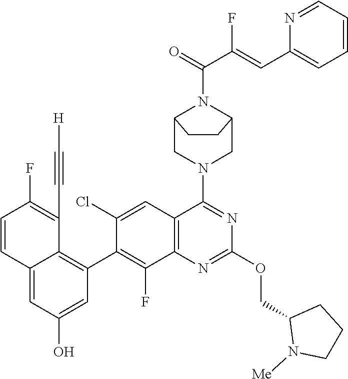 [SMILES: C#Cc1c(F)ccc2cc(O)cc(-c3c(Cl)cc4c(N5CC6CCC(C5)N6C(=O)/C(F)=C/c5ccccn5)nc(OC[C@@H]5CCCN5C)nc4c3F)c12]

### Example 20

### (2Z)-1-{3-[6-chloro-7-(8-ethynyl-7-fluoro-3-hydroxynaphthalen-1-yl)-8-fluoro-2-{[(2S)-1-methylpyrrolidin-2-yl]methoxy}quinazolin-4-yl]-3,8-diazabicyclo[3.2.1]octan-8-yl}-2-fluoro-3-(pyridin-2-yl)prop-2-en-1-one

4-(4-((1R,5S)-3,8-Diazabicyclo[3.2.1]octan-3-yl)-6-chloro-8-fluoro-2-(((S)-1-methylpyrrolidin-2-yl)methoxy)quinazolin-7-yl)-5-ethynyl-6-fluoronaphthalen-2-ol (Intermediate 2, 20 mg, 0.034 mmol) and (Z)-2-fluoro-3-(pyridin-2-yl)acrylic acid (Intermediate 27, 5.6 mg, 0.034 mmol) were combined as solids and dissolved in DMF (1.0 mL). 1-Methylimidazole (27 μL, 0.34 mmol) was added followed by chloro-N,N,N′,N′-tetramethylformamidinium hexafluorophosphate (9.5 mg, 0.034 mmol). The reaction mixture was stirred at room temperature for 5 min. A saturated aqueous solution of ammonium hydroxide (50 μL) was added and the reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 71% 5:95 MeCN:H₂O with 10 mM AA/29% 95:5 MeCN:H₂O with 10 mM AA→100% 95:5 MeCN:H₂O with 10 mM AA; λ=220 nm) to provide the desired product as a mixture of atropisomers. This mixture was purified by chiral SFC (column: Chiralcel OD-H, 30 mm×250 mm, 5 m particles; flow rate: 85 mL/min; column temperature: 40° C., isocratic: 55% CO₂:45% MeOH with 0.1% NH₄OH; λ=230 nm) to provide two products, each as a single diastereomer of unknown absolute stereochemistry: Isomer 1 (1.0 mg, 0.001 mmol, 4% yield) and Isomer 2 (2.4 mg, 0.003 mmol, 10% yield). Isomer 1: LC/MS (ESI) m/z: [M+H]⁺ calcd for C₄₀H₃₅ClF₃N₆O₃ 739.2. found 739.2. Insufficient material was obtained for NMR analysis Isomer 2: LC/MS (ESI) m/z: [M+H]⁺ calcd for C₄₀H₃₅ClF₃N₆O₃ 739.2. found 739.1. ¹H NMR (500 MHz, DMSO-d₆) δ 10.11 (br s, 1H), 8.66 (br d, J=4.7 Hz, 1H), 8.20 (d, J=8.9 Hz, 1H), 7.99-7.87 (m, 3H), 7.82 (d, J=7.9 Hz, 1H), 7.48 (t, J=9.0 Hz, 1H), 7.42-7.34 (m, 2H), 7.29 (d, J=2.0 Hz, 1H), 6.81 (d, J=37.8 Hz, 1H), 4.79-4.73 (m, 2H), 4.54-4.43 (m, 2H), 4.40 (dd, J=10.5, 4.8 Hz, 1H), 4.21 (dd, J=10.6, 6.3 Hz, 1H), 3.91-3.86 (m, 1H), 3.82-3.61 (m, 3H), 3.11-3.07 (m, 2H), 2.99-2.93 (m, 1H), 2.64-2.57 (m, 1H), 2.38 (s, 3H), 2.19 (q, J=8.8 Hz, 1H), 2.00-1.91 (m, 2H), 1.85-1.77 (m, 2H), 1.72-1.60 (m, 3H).

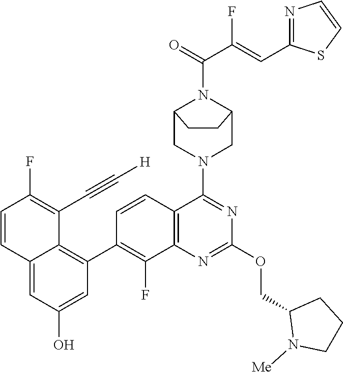 [SMILES: C#Cc1c(F)ccc2cc(O)cc(-c3ccc4c(N5CC6CCC(C5)N6C(=O)/C(F)=C/c5nccs5)nc(OC[C@@H]5CCCN5C)nc4c3F)c12]

### Example 21

### (Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(thiazol-2-yl)prop-2-en-1-one

4-(4-((1R,5S)-3,8-Diazabicyclo[3.2.1]octan-3-yl)-8-fluoro-2-(((S)-1-methylpyrrolidin-2-yl)methoxy)quinazolin-7-yl)-5-ethynyl-6-fluoronaphthalen-2-ol (Intermediate 11, 15 mg, 0.027 mmol) and (Z)-2-fluoro-3-(thiazol-2-yl)acrylic acid (Intermediate 25, 4.7 mg, 0.027 mmol) were combined as solids and dissolved in DMF (1 mL). 1-Methylimidazole (22 μL, 0.27 mmol) was added followed by chloro-N,N,N′,N′-tetramethylformamidinium hexafluorophosphate (7.6 mg, 0.027 mmol). The reaction mixture was stirred at room temperature for 5 min. A saturated aqueous solution of ammonium hydroxide (100 μL) was added and the reaction mixture was stirred for 5 min. The reaction mixture was directly purified by reverse phase HPLC (column: Xbridge C18, 19 mm×200 mm, 5 μm particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (8.1 mg, 0.011 mmol, 42% yield). LC/MS (ESI) m/z: [M+H]⁺ calcd for C₃₈H₃₄F₃N₆O₃S 711.2; found 711.1. NMR: a water suppression method was used, which also hides the broadened naphthol proton ¹H NMR (500 MHz, DMSO-d₆) δ 8.06-7.99 (m, 2H), 7.96 (dd, J=8.7, 6.1 Hz, 1H), 7.78 (d, J=8.7 Hz, 1H), 7.46 (dd, J=9.0, 8.5 Hz, 1H), 7.36 (d, J=2.4 Hz, 1H), 7.23 (dd, J=8.5, 7.6 Hz, 2H), 7.20 (d, J=36.8 Hz, 1H), 7.07 (d, J=2.4 Hz, 1H), 4.86-4.75 (m, 2H), 4.58-4.49 (m, 1H), 4.48-4.36 (m, 2H), 4.34-4.17 (m, 1H), 3.85 (s, 1H), 3.76-3.60 (m, 2H), 3.47-3.34 (m, 1H), 2.45 (br s, 3H), 2.38-2.23 (m, 11H) 2.07-1.91 (m, 4H), 1.77-1.64 (m, 3H).

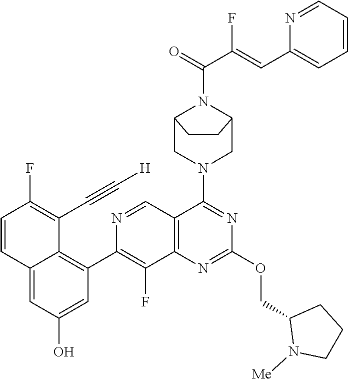 [SMILES: C#Cc1c(F)ccc2cc(O)cc(-c3ncc4c(N5CC6CCC(C5)N6C(=O)/C(F)=C/c5ccccn5)nc(OC[C@@H]5CCCN5C)nc4c3F)c12]

### Example 22

### (Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one

4-(4-((1R,5S)-3,8-Diazabicyclo[3.2.1]octan-3-yl)-8-fluoro-2-(((S)-1-methylpyrrolidin-2-yl)methoxy)quinazolin-7-yl)-5-ethynyl-6-fluoronaphthalen-2-ol (Intermediate 11, 15 mg, 0.027 mmol) and (Z)-2-fluoro-3-(pyridin-2-yl)acrylic acid (Intermediate 27, 4.5 mg, 0.027 mmol) were combined as solids and dissolved in DMF (1 mL). 1-Methylimidazole (22 μL, 0.27 mmol) was added followed by chloro-N,N,N′,N′-tetramethylformamidinium hexafluorophosphate (7.6 mg, 0.027 mmol). The reaction mixture was stirred at room temperature for 5 min. A saturated aqueous solution of ammonium hydroxide (100 μL) was added and the reaction mixture was stirred for 5 min. The reaction mixture was directly purified by reverse phase HPLC (column: Xbridge C18, 19 mm×200 mm, 5 μm particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (8.2 mg, 0.012 mmol, 43% yield). LC/MS (ESI) m/z: [M+H]⁺ calcd for C₄₀H₃₆F₃N₆O₃ 705.3. found 705.2. NMR: a water suppression method was used, which also hides the broadened naphthol proton ¹H NMR (500 MHz, DMSO-d₆) δ 8.66 (br d, J=4.7 Hz, 1H), 7.96 (dd, J=8.7, 6.3 Hz, 1H), 7.90 (dd, J=7.9, 7.5 Hz, 1H), 7.81 (d, J=7.9 Hz, 1H), 7.77 (br d, J=8.7 Hz, 1H), 7.46 (dd, J=9.5, 8.0 Hz, 1H), 7.39 (dd, J=7.5, 4.3 Hz, 1H), 7.36 (d, J=2.1 Hz, 1H), 7.22 (dd, J=8.0, 7.0 Hz, 1H), 7.07 (d, J=2.1 Hz, 1H), 6.81 (d, J=37.8 Hz, 1H), 4.85-4.74 (m, 2H), 4.56-4.46 (m, 1H), 4.45-4.33 (m, 2H), 4.22-4.11 (m, 1H), 3.86 (s, 1H), 3.74-3.59 (m, 2H), 2.99-2.91 (m, 1H), 2.62-2.55 (m, 1H), 2.36 (s, 3H), 2.22-2.14 (m, 1H), 2.04-1.92 (m, 4H), 1.72-1.59 (m, 3H).

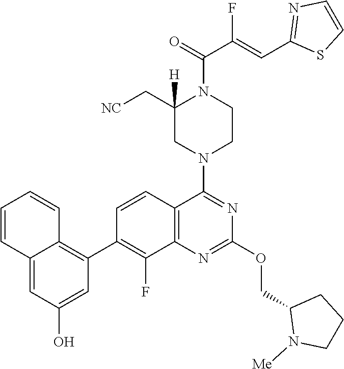 [SMILES: CN1CCC[C@H]1COc1nc(N2CCN(C(=O)/C(F)=C/c3nccs3)[C@@H](CC#N)C2)c2ccc(-c3cc(O)cc4ccccc34)c(F)c2n1]

### Example 23

### 2-((S)-1-((Z)-2-fluoro-3-(thiazol-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile

2-((S)-4-(8-Fluoro-7-(3-hydroxynaphthalen-1-yl)-2-(((S)-1-methylpyrrolidin-2-yl)methoxy)quinazolin-4-yl)piperazin-2-yl)acetonitrile (Intermediate 4, 15 mg, 0.028 mmol) and (Z)-2-fluoro-3-(thiazol-2-yl)acrylic acid (Intermediate 25, 4.5 mg, 0.028 mmol) were combined as solids and dissolved in DMF (1 mL). 1-Methylimidazole (23 μL, 0.28 mmol) was added followed by chloro-N,N,N′,N′-tetramethylformamidinium hexafluorophosphate (7.6 mg, 0.028 mmol). The reaction mixture was stirred at room temperature for 5 min. A saturated aqueous solution of ammonium hydroxide (100 μL) was added and the reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 84% 5:95 MeCN:H₂O with 10 mM AA/16% 95:5 MeCN:H₂O with 10 mM AA→100% 95:5 MeCN:H₂O with 10 mM AA; λ=220 nm) to provide the desired product (3.3 mg, 0.005 mmol, 17% yield). LC/MS (ESI) m/z: [M+H]⁺ calcd for C₃₆H₃₄F₂N₇O₃S 676.3. found 676.1. NMR: a water suppression method was used ¹H NMR (500 MHz, DMSO-d₆) δ 8.66 (br d, J=4.1 Hz, 1H), 7.98 (br d, J=8.3 Hz, 1H), 7.93-7.85 (m, 1H), 7.83-7.80 (m, 1H), 7.80-7.78 (m, 1H), 7.47-7.42 (m, 1H), 7.42-7.36 (m, 2H), 7.35-7.31 (m, 1H), 7.27 (d, J=1.7 Hz, 1H), 7.11 (d, J=1.7 Hz, 1H), 6.65 (d, J=38.9 Hz, 1H), 5.11-4.81 (m, 1H), 4.48-4.29 (m, 3H), 4.28-4.19 (m, 1H), 3.69-3.58 (m, 1H), 3.20-3.07 (m, 1H), 3.04-2.92 (m, 1H), 2.67-2.56 (m, 1H), 2.37 (s, 3H), 2.22-2.15 (m, 1H), 2.00-1.93 (m, 1H), 1.72-1.61 (m, 3H)

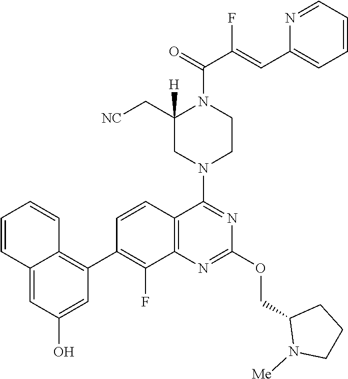 [SMILES: CN1CCC[C@H]1COc1nc(N2CCN(C(=O)/C(F)=C/c3ccccn3)[C@@H](CC#N)C2)c2ccc(-c3cc(O)cc4ccccc34)c(F)c2n1]

### Example 24

### 2-((S)-1-((Z)-2-fluoro-3-(pyridin-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile

2-((S)-4-(8-Fluoro-7-(3-hydroxynaphthalen-1-yl)-2-(((S)-1-methylpyrrolidin-2-yl)methoxy)quinazolin-4-yl)piperazin-2-yl)acetonitrile (Intermediate 4, 15 mg, 0.028 mmol) and (Z)-2-fluoro-3-(pyridin-2-yl)acrylic acid (Intermediate 27, 4.8 mg, 0.028 mmol) were combined as solids and dissolved in DMF (1 mL). 1-Methylimidazole (23 μL, 0.28 mmol) was added followed by chloro-N,N,N′,N′-tetramethylformamidinium hexafluorophosphate (7.6 mg, 0.028 mmol). The reaction mixture was stirred at room temperature for 5 min. A saturated aqueous solution of ammonium hydroxide (100 μL) was added and the reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 88% 5:95 MeCN:H₂O with 0.1% TFA/12% 95:5 MeCN:H₂O with 0.1% TFA→100% 95:5 MeCN:H₂O with 0.1% TFA; λ=220 nm) to provide the desired product as a bis-TFA salt (10.6 mg, 0.012 mmol, 42% yield) LC/MS (ESI) m/z: [M+H]⁻ calcd for C₃₈H₃₆F₂N₇O₃ 682.2. found 682.2. NMR: ¹H NMR (500 MHz, DMSO-d₆) δ 9.84 (br s, 1H), 8.05-7.99 (m, 3H), 7.82 (d, J=8.4 Hz, 1H), 7.45 (t, J=7.6 Hz, 1H), 7.42-7.34 (m, 2H), 7.28 (d, J=1.7 Hz, 1H), 7.27-7.21 (m, 1H), 7.11 (d, J=37.3 Hz, 1H), 7.11 (d, J=1.7 Hz, 1H), 5.01-4.89 (m, 1H), 4.76 (dd, J=12.5, 3.1 Hz, 1H), 4.62 (dd, J=12.5, 6.6 Hz, 1H), 4.47-4.36 (m, 1H), 3.79 (br d, J=3.2 Hz, 1H), 3.19-3.06 (m, 2H), 3.01-2.93 (m, 3H), 2.31-2.21 (m, 1H), 2.11-2.02 (m, 1H), 1.96-1.85 (m, 2H)

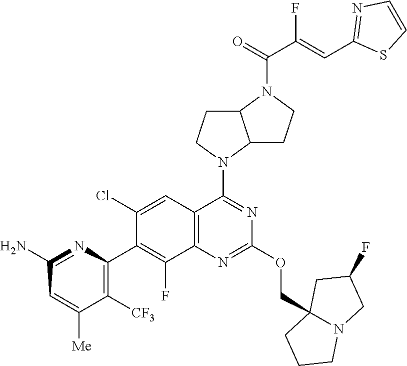

### Example 25

### (Z)-1-(4-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)hexahydropyrrolo[3,2-b]pyrrol-1(2H)-yl)-2-fluoro-3-(thiazol-2-yl)prop-2-en-1-one

(5M)-6-((7R)-6-Chloro-8-fluoro-2-(((2R,7aR)-2-fluorotetrahydro-1H-pyrrolizin-7a(5H)-yl)methoxy)-4-(hexahydropyrrolo[3,2-b]pyrrol-1(2H)-yl)quinazolin-7-yl)-4-methyl-5-(trifluoromethyl)pyridin-2-amine (Intermediate 20, 10 mg, 0.016 mmol) (as a mixture of two cis-diastereomers) and (Z)-2-fluoro-3-(thiazol-2-yl)acrylic acid (Intermediate 25, 8.3 mg, 0.048 mmol) were combined as solids and dissolved in DMF (0.3 mL). 1-Methylimidazole (8 μL, 0.096 mmol) was added followed by chloro-N,N,N′,N′-tetramethylformamidinium hexafluorophosphate (13.5 mg, 0.048 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide two products as single diastereomers of unknown absolute stereochemistry: Isomer 1 (4 mg, 0.005 mmol, 32% yield) and Isomer 2 (4.1 mg, 0.005 mmol, 33% yield). Isomer 1: LC/MS (ESI) m/z: [M+H]⁺ calcd for C₃₅H₃₄ClF₆N₈O₃S 779.2. found 779.2. NMR: ¹H NMR (500 MHz, DMSO-d₆) δ 8.11 (br s, 1H), 8.04-7.97 (m, 2H), 7.24 (d, J=37.2 Hz, 1H), 6.84 (s, 2H), 6.50 (s, 1H), 5.52-5.29 (m, 1H), 5.17-5.04 (m, 1H), 4.76-4.60 (m, 1H), 4.34-4.03 (m, 3H), 4.01-3.87 (m, 1H), 3.82-3.69 (m, 1H), 3.19-2.83 (m, 1H), 2.45-2.28 (m, 6H), 2.26-2.07 (m, 3H), 2.06-1.83 (m, 3H), 1.82-1.65 (m, 1H). Isomer 2: NMR: ¹H NMR (500 MHz, DMSO-d₆) δ 8.12 (br s, 1H), 8.05-7.98 (m, 2H), 7.24 (d, J=36.6 Hz, 1H), 6.86 (br s, 2H), 6.49 (s, 1H), 5.50-5.28 (m, 1H), 5.17-5.06 (m, 1H), 4.77-4.64 (m, 1H), 4.35-4.11 (m, 2H), 4.10-4.02 (m, 1H), 4.00-3.91 (m, 1H), 3.86-3.71 (m, 1H), 3.11-2.75 (m, 1H), 2.43-2.23 (m, 6H), 2.22-2.06 (m, 3H), 2.04-1.82 (m, 3H), 1.81-1.65 (m, 1H).

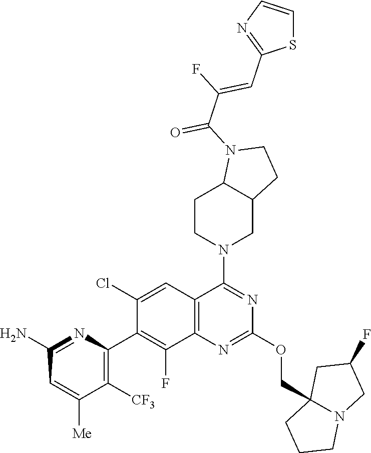

### Example 26

### (Z)-1-(5-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-1H-pyrrolo[3,2-c]pyridin-1-yl)-2-fluoro-3-(thiazol-2-yl)prop-2-en-1-one

(5M)-6-(6-Chloro-8-fluoro-2-(((2R,7aR)-2-fluorotetrahydro-1H-pyrrolizin-7a(5H)-yl)methoxy)-4-(octahydro-5H-pyrrolo[3,2-c]pyridin-5-yl)quinazolin-7-yl)-4-methyl-5-(trifluoromethyl)pyridin-2-amine (Intermediate 19, 10 mg, 0.016 mmol) (as a mixture of two cis-diastereomers) and (Z)-2-fluoro-3-(thiazol-2-yl)acrylic acid (Intermediate 25, 8.3 mg, 0.048 mmol) were combined as solids and dissolved in DMF (0.3 mL). 1-Methylimidazole (8 μL, 0.096 mmol) was added followed by chloro-N,N,N′,N′-tetramethylformamidinium hexafluorophosphate (13.5 mg, 0.048 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to provide the desired product as a mixture of two diastereomers. This mixture was purified by chiral SFC (column: Chiralcel OD-H, 30 mm×250 mm, 5 m particles; flow rate: 85 mL/min; column temperature: 40° C., isocratic: 70% CO₂:30% MeOH with 0.1% NH₄OH; λ=295 nm) to provide two products, each as a single diastereomer of unknown absolute stereochemistry: Isomer 1 (2.5 mg, 0.003 mmol, 20% yield) and Isomer 2 (1.8 mg, 0.002 mmol, 14% yield). Isomer 1: LC/MS (ESI) m/z: [M+H]⁺ calcd for C₃₆H₃₅ClF₆N₈O₃S 793.2. found 773.0. NMR: ¹H NMR (500 MHz, DMSO-d₆) δ 8.04-7.97 (m, 2H), 7.91 (br s, 1H), 7.19 (d, J=36.6 Hz, 1H), 6.84 (br s, 2H), 6.50 (s, 1H), 5.57-5.32 (m, 1H), 4.59-4.15 (m, 3H), 4.12-3.92 (m, 2H), 3.89-3.78 (m, 1H), 3.72-3.50 (m, 2H), 2.75-2.57 (m, 1H), 2.37 (br s, 3H), 2.35-2.30 (m, 1H), 2.21-1.82 (m, 8H). Isomer 2: LC/MS (ESI) m/z: [M+H]⁺ calcd for C₃₆H₃₆ClF₆N₈O₃S 793.2. found 793.0. NMR: this sample was contaminated with TFA at some point during the purification process. ¹H NMR (500 MHz, DMSO-d₆) δ 8.00 (br d, J=10.3 Hz, 2H), 7.94 (br s, 1H), 7.19 (d, J=36.8 Hz, 1H), 6.86 (br s, 2H), 6.50 (s, 1H), 5.59-5.43 (m, 1H), 4.67-4.53 (m, 2H), 4.45-4.19 (m, 1H), 4.16-4.03 (m, 1H), 3.99-3.91 (m, 2H), 3.87-3.77 (m, 2H), 2.70-2.57 (m, 2H), 2.46-2.40 (m, 1H), 2.37 (br s, 3H), 2.34-2.28 (m, 1H), 2.28-2.09 (m, 4H), 2.09-1.87 (m, 4H).

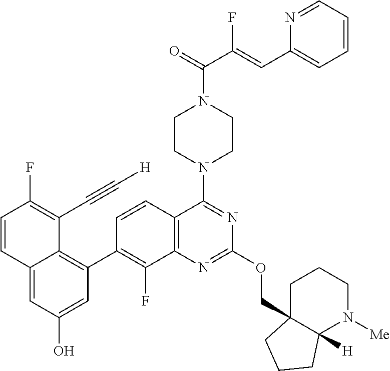 [SMILES: C#Cc1c(F)ccc2cc(O)cc(-c3ccc4c(N5CCN(C(=O)/C(F)=C/c6ccccn6)CC5)nc(OC[C@]56CCC[C@H]5N(C)CCC6)nc4c3F)c12]

### Example 27

### (Z)-1-((1R,5S)-3-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)quinazolin-4-yl)-3,8-diazabicyclo[3.2.1]octan-8-yl)-2-fluoro-3-(pyridin-2-yl)prop-2-en-1-one

4-(4-((1R,5S)-3,8-Diazabicyclo[3.2.1]octan-3-yl)-8-fluoro-2-(((4aS,7aR)-1-methyloctahydro-4aH-cyclopenta[b]pyridin-4a-yl)methoxy)quinazolin-7-yl)-5-ethynyl-6-fluoronaphthalen-2-ol (Intermediate 11, 3 mg, 0.005 mmol) and (Z)-2-fluoro-3-(pyridin-2-yl)acrylic acid (Intermediate 27, 2.5 mg, 0.015 mmol) were combined as solids and dissolved in DMF (0.3 mL). 1-Methylimidazole (2 μL, 0.03 mmol) was added followed by chloro-N,N,N′,N′-tetramethylformamidinium hexafluorophosphate (4 mg, 0.015 mmol). The reaction mixture was stirred at room temperature for 5 min. A saturated aqueous solution of ammonium hydroxide (100 L) was added and the reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (3.5 mg, 0.005 mmol, 94% yield) LC/MS (ESI) m/z: [M+H]⁺ calcd for C₃₆H₃₆ClF₆N₈O₃S 759.3. found 759.1. NMR: ¹H NMR (500 MHz, DMSO-d₆) δ 8.66 (d, J=4.6 Hz, 1H), 7.96 (dd, J=8.8, 6.2 Hz, 1H), 7.90 (dd, J=7.9, 7.5 Hz, 1H), 7.81 (d, J=7.9 Hz, 1H), 7.78 (d, J=8.8 Hz, 1H), 7.46 (dd, J=9.0, 8.0 Hz, 1H), 7.39 (dd, J=7.5, 4.6 Hz, 1H), 7.36 (d, J=2.5 Hz, 1H), 7.22 (dd, J=8.0, 7.0 Hz, 1H), 7.08 (d, J=2.4 Hz, 1H), 6.81 (d, J=37.9 Hz, 1H), 4.85-4.74 (m, 2H), 4.54-4.36 (m, 3H), 4.12 (dd, J=10.7, 3.8 Hz, 1H), 3.86 (d, J=5.9 Hz, 1H), 3.67-3.60 (m, 2H), 2.74-2.66 (m, 1H), 2.49-2.43 (m, 1H), 2.30-2.22 (m, 1H), 2.19 (br s, 3H), 2.04-1.91 (m, 4H), 1.87-1.79 (m, 1H), 1.75-1.68 (m, 1H), 1.67-1.54 (m, 6H), 1.53-1.46 (m, 1H), 1.43-1.34 (m, 1H).

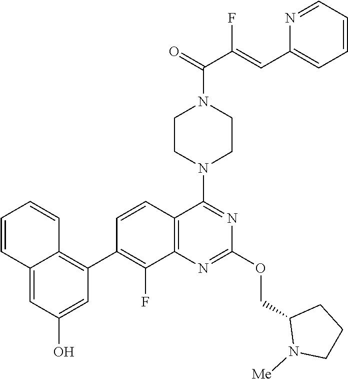 [SMILES: CN1CCC[C@H]1COc1nc(N2CCN(C(=O)/C(F)=C/c3ccccn3)CC2)c2ccc(-c3cc(O)cc4ccccc34)c(F)c2n1]

### Example 28

### (S,Z)-2-fluoro-1-(4-(8-fluoro-7-(3-hydroxynaphthalen-1-yl)-2-((1-methylpyrrolidin-2-yl)methoxy)quinazolin-4-yl)piperazin-1-yl)-3-(pyridin-2-yl)prop-2-en-1-one

(S)-4-(8-Fluoro-2-((1-methylpyrrolidin-2-yl)methoxy)-4-(piperazin-1-yl)quinazolin-7-yl)naphthalen-2-ol (Intermediate 3, 15 mg, 0.031 mmol) and (Z)-2-fluoro-3-(pyridin-2-yl)acrylic acid (Intermediate 27, 5.1 mg, 0.031 mmol) were combined as solids and dissolved in DMF (1 mL). 1-Methylimidazole (25 μL, 0.31 mmol) was added followed by chloro-N,N,N′,N′-tetramethylformamidinium hexafluorophosphate (8.6 mg, 0.031 mmol). The reaction mixture was stirred at room temperature for 5 min. A saturated aqueous solution of ammonium hydroxide (100 μL) was added and the reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 94% 5:95 MeCN:H₂O with 0.1% TFA/6% 95:5 MeCN:H₂O with 0.1% TFA→100% 95:5 MeCN:H₂O with 0.1% TFA; λ=220 nm) to provide the desired product as the bis-TFA salt (21 mg, 0.024 mmol, 79% yield). LC/MS (ESI) m/z: [M+H]⁺ calcd for C₃₆H₃₅F₂N₆O₃ 637.3. found 637.0. NMR: ¹H NMR (500 MHz, DMSO-d₆) δ 9.87 (br s, 1H), 8.66 (br d, J=4.9 Hz, 1H), 8.00 (d, J=8.5 Hz, 1H), 7.95-7.89 (m, 1H), 7.81 (t, J=8.0 Hz, 2H), 7.48-7.42 (m, 1H), 7.42-7.36 (m, 3H), 7.30-7.21 (m, 3H), 7.11 (d, J=2.2 Hz, 1H), 6.66 (d, J=38.7 Hz, 1H), 4.83-4.72 (m, 1H), 4.66-4.58 (m, 1H), 4.09-3.99 (m, 4H), 3.96-3.81 (m, 4H), 3.18-3.10 (m, 2H), 2.97 (br s, 3H), 2.31-2.23 (m, 1H), 2.11 (br d, J=3.7 Hz, 1H), 1.97-1.85 (m, 2H)

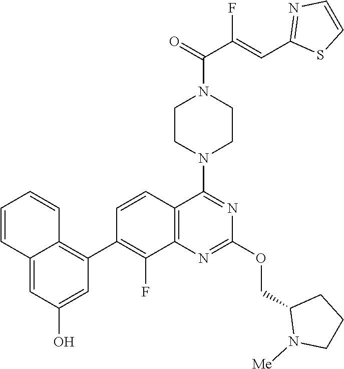 [SMILES: CN1CCC[C@H]1COc1nc(N2CCN(C(=O)/C(F)=C/c3nccs3)CC2)c2ccc(-c3cc(O)cc4ccccc34)c(F)c2n1]

### Example 29

### (S,Z)-2-fluoro-1-(4-(8-fluoro-7-(3-hydroxynaphthalen-1-yl)-2-((1-methylpyrrolidin-2-yl)methoxy)quinazolin-4-yl)piperazin-1-yl)-3-(thiazol-2-yl)prop-2-en-1-one

(S)-4-(8-Fluoro-2-((1-methylpyrrolidin-2-yl)methoxy)-4-(piperazin-1-yl)quinazolin-7-yl)naphthalen-2-ol (Intermediate 3, 15 mg, 0.031 mmol) and (Z)-2-fluoro-3-(thiazol-2-yl)acrylic acid (Intermediate 25, 5.3 mg, 0.031 mmol) were combined as solids and dissolved in DMF (1 mL). 1-Methylimidazole (25 μL, 0.31 mmol) was added followed by chloro-N,N,N′,N′-tetramethylformamidinium hexafluorophosphate (8.6 mg, 0.031 mmol). The reaction mixture was stirred at room temperature for 5 min. A saturated aqueous solution of ammonium hydroxide (100 μL) was added and the reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 82% 5:95 MeCN:H₂O with 10 mM AA/18% 95:5 MeCN:H₂O with 10 mM AA→100% 95:5 MeCN:H₂O with 10 mM AA; λ=220 nm) to provide the desired product (14.9 mg, 0.023 mmol, 75% yield). LC/MS (ESI) m/z: [M+H]⁺ calcd for C₃₄H₃₃F₂N₆O₃S 637.3. found 637.0. NMR: ¹H NMR (500 MHz, DMSO-d₆) δ 9.99 (br s, 1H), 8.02-7.97 (m, 2H), 7.94 (d, J=8.5 Hz, 1H), 7.80 (d, J=8.5 Hz, 1H), 7.46-7.38 (m, 2H), 7.32 (t, J=7.7 Hz, 1H), 7.28-7.22 (m, 2H), 7.11 (d, J=2.2 Hz, 1H), 7.09 (d, J=37.8 Hz, 1H), 4.45-4.39 (m, 1H), 4.27-4.19 (m, 1H), 4.03-3.95 (m, 4H), 3.93-3.83 (m, 4H), 3.00-2.94 (m, 1H), 2.68-2.61 (m, 1H), 2.38 (s, 3H), 2.26-2.17 (m, 1H), 2.03-1.92 (m, 1H), 1.74-1.60 (m, 3H)

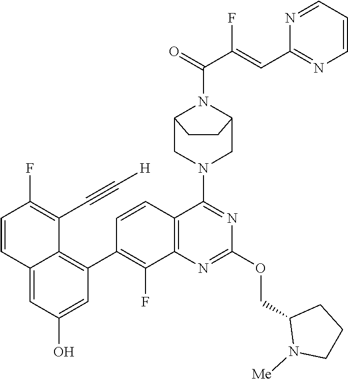 [SMILES: C#Cc1c(F)ccc2cc(O)cc(-c3ccc4c(N5CC6CCC(C5)N6C(=O)/C(F)=C/c5ncccn5)nc(OC[C@@H]5CCCN5C)nc4c3F)c12]

### Example 30

### (Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(pyrimidin-2-yl)prop-2-en-1-one

4-(4-((1R,5S)-3,8-Diazabicyclo[3.2.1]octan-3-yl)-8-fluoro-2-(((S)-1-methylpyrrolidin-2-yl)methoxy)quinazolin-7-yl)-5-ethynyl-6-fluoronaphthalen-2-ol (Intermediate 11, 15 mg, 0.027 mmol) and (Z)-2-fluoro-3-(pyrimidin-2-yl)acrylic acid (Intermediate 26, 4.5 mg, 0.027 mmol) were combined as solids and dissolved in DMF (1 mL). 1-methylimidazole (22 μL, 0.27 mmol) was added followed by chloro-N,N,N′,N′-tetramethylformamidinium hexafluorophosphate (7.6 mg, 0.027 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 85% 5:95 MeCN:H₂O with 10 mM AA/15% 95:5 MeCN:H₂O with 10 mM AA→100% 95:5 MeCN:H₂O with 10 mM AA; λ=220 nm) to provide the desired product (9.5 mg, 0.014 mmol, 50% yield) LC/MS (ESI) m/z: [M+H]⁺ calcd for C₃₉H₃₅F₃N₇O₃ 706.3. found 706.0. NMR: ¹H NMR (500 MHz, DMSO-d₆) δ 10.15 (br s, 1H), 8.90 (d, J=4.9 Hz, 2H), 7.96 (dd, J=8.7, 6.1 Hz, 1H), 7.78 (d, J=8.7 Hz, 1H), 7.46 (dd, J=9.5, 8.0 Hz, 2H), 7.45 (t, J=4.9 Hz, 1H), 7.36 (d, J=2.3 Hz, 1H), 7.23 (dd, J=8.0, 7.0 Hz, 1H), 7.07 (d, J=2.3 Hz, 1H), 6.78 (d, J=34.6 Hz, 1H), 4.86-4.74 (m, 2H), 4.56-4.48 (m, 1H), 4.46-4.37 (m, 2H), 4.28-4.17 (m, 1H), 3.85 (s, 1H), 3.72-3.62 (m, 2H), 3.05-2.99 (m, 1H), 2.77-2.67 (m, 1H), 2.42 (br s, 3H), 2.33-2.23 (m, 1H), 2.04-1.91 (m, 4H), 1.77-1.60 (m, 3H).

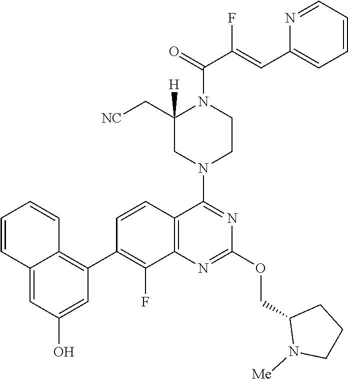 [SMILES: CN1CCC[C@H]1COc1nc(N2CCN(C(=O)/C(F)=C/c3ccccn3)[C@@H](CC#N)C2)c2ccc(-c3cc(O)cc4ccccc34)c(F)c2n1]

### Example 31

### 2-((S)-1-((Z)-2-fluoro-3-(pyrimidin-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile

2-((S)-4-(8-Fluoro-7-(3-hydroxynaphthalen-1-yl)-2-(((S)-1-methylpyrrolidin-2-yl)methoxy)quinazolin-4-yl)piperazin-2-yl)acetonitrile (Intermediate 4, 15 mg, 0.028 mmol) and (Z)-2-fluoro-3-(pyrimidin-2-yl)acrylic acid (Intermediate 26, 4.8 mg, 0.028 mmol). 1-Methylimidazole (23 μL, 0.28 mmol) was added followed by chloro-N,N,N′,N′-tetramethylformamidinium hexafluorophosphate (8 mg, 0.028 mmol). The reaction mixture was stirred at room temperature for 5 min. A saturated aqueous solution of ammonium hydroxide (100 L) was added and the reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 91% 5:95 MeCN:H₂O with 0.1% TFA/9% 95:5 MeCN:H₂O with 0.1% TFA→100% 95:5 MeCN:H₂O with 0.1% TFA; λ=220 nm) to provide the desired product (15.7 mg, 0.018 mmol, 63% yield). LC/MS (ESI) m/z: [M+H]⁺ calcd for C₃₇H₃₅F₂N₈O₃ 677.3. found 677.0. NMR: a water suppression method was used ¹H NMR (500 MHz, DMSO-d₆) δ 8.66 (br d, J=4.1 Hz, 1H), 7.98 (br d, J=8.3 Hz, 1H), 7.93-7.85 (m, 1H), 7.83-7.80 (m, 1H), 7.80-7.78 (m, 1H), 7.47-7.42 (m, 1H), 7.42-7.36 (m, 2H), 7.35-7.31 (m, 1H), 7.27 (d, J=1.7 Hz, 1H), 7.11 (d, J=1.7 Hz, 1H), 6.65 (d, J=38.9 Hz, 1H), 5.11-4.81 (m, 1H), 4.48-4.29 (m, 3H), 4.28-4.19 (m, 1H), 3.69-3.58 (m, 1H), 3.20-3.07 (m, 1H), 3.04-2.92 (m, 1H), 2.67-2.56 (m, 1H), 2.37 (s, 3H), 2.22-2.15 (m, 1H), 2.00-1.93 (m, 1H), 1.72-1.61 (m, 3H)

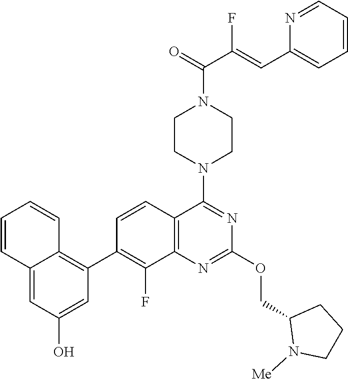 [SMILES: CN1CCC[C@H]1COc1nc(N2CCN(C(=O)/C(F)=C/c3ccccn3)CC2)c2ccc(-c3cc(O)cc4ccccc34)c(F)c2n1]

### Example 32

### (S,Z)-2-fluoro-1-(4-(8-fluoro-7-(3-hydroxynaphthalen-1-yl)-2-((1-methylpyrrolidin-2-yl)methoxy)quinazolin-4-yl)piperazin-1-yl)-3-(pyrimidin-2-yl)prop-2-en-1-one

(S)-4-(8-Fluoro-2-((1-methylpyrrolidin-2-yl)methoxy)-4-(piperazin-1-yl)quinazolin-7-yl)naphthalen-2-ol (Intermediate 3, 15 mg, 0.031 mmol) and (Z)-2-fluoro-3-(pyrimidin-2-yl)acrylic acid (Intermediate 26, 5.2 mg, 0.031 mmol) were combined as solids and dissolved in DMF (1 mL). 1-Methylimidazole (25 μL, 0.31 mmol) was added followed by chloro-N,N,N′,N′-tetramethylformamidinium hexafluorophosphate (8.6 mg, 0.031 mmol) The reaction mixture was stirred at room temperature for 5 min. A saturated aqueous solution of ammonium hydroxide (100 μL) was added and the reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 94% 5:95 MeCN:H₂O with 0.1% TFA/6% 95:5 MeCN:H₂O with 0.1% TFA→100% 95:5 MeCN:H₂O with 0.1% TFA; λ=220 nm) to provide the desired product as the bis-TFA salt (19.2 mg, 0.022 mmol, 72% yield) LC/MS (ESI) m/z: [M+H]⁺ calcd for C₃₅H₃₄F₂N₇O₃ 638.3. found 638.1. NMR: ¹H NMR (500 MHz, DMSO-d₆) δ 9.84 (br s, 1H), 8.90 (br d, J=4.7 Hz, 2H), 8.02 (br d, J=8.0 Hz, 1H), 7.82 (br d, J=8.0 Hz, 1H), 7.46 (t, J=4.7 Hz, 1H), 7.48-7.42 (m, 1H), 7.42-7.35 (m, 2H), 7.29-7.22 (m, 2H), 7.11 (s, 1H), 6.63 (d, J=35.9 Hz, 1H), 5.08-4.82 (m, 1H), 4.79-4.72 (m, 1H), 4.66-4.59 (m, 1H), 4.49-4.36 (m, 2H), 4.17-3.93 (m, 1H), 3.87-3.79 (m, 1H), 3.64-3.59 (m, 1H), 3.18-3.09 (m, 3H), 2.97 (br s, 3H), 2.33-2.23 (m, 1H), 2.13 (s, 1H), 2.01-1.85 (m, 2H).

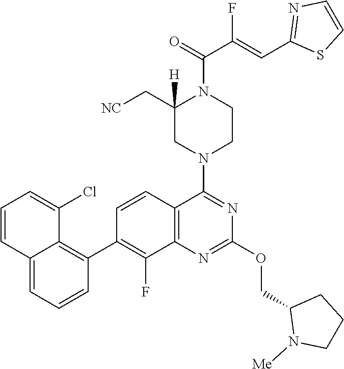 [SMILES: CN1CCC[C@H]1COc1nc(N2CCN(C(=O)/C(F)=C/c3nccs3)[C@@H](CC#N)C2)c2ccc(-c3cccc4cccc(Cl)c34)c(F)c2n1]

### Example 33

### 2-((S)-1-((Z)-2-fluoro-3-(thiazol-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile

2-((S)-4-(8-Fluoro-7-(8-chloronaphthalen-1-yl)-2-(((S)-1-methylpyrrolidin-2-yl)methoxy)quinazolin-4-yl)piperazin-2-yl)acetonitrile (Intermediate 5, 15 mg, 0.028 mmol) and (Z)-2-fluoro-3-(thiazol-2-yl)acrylic acid (Intermediate 25, 4.5 mg, 0.028 mmol) were combined as solids and dissolved in DMF (1 mL). 1-Methylimidazole (23 μL, 0.28 mmol) was added followed by chloro-N,N,N′,N′-tetramethylformamidinium hexafluorophosphate (7.6 mg, 0.028 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (6.3 mg, 0.009 mmol, 30% yield). LC/MS (ESI) m/z: [M+H]⁺ calcd for C₃₆H₃₃ClF₂N₇O₂S 700.2. found 700.2. ¹H NMR (500 MHz, DMSO-d₆) δ 8.17 (d, J=8.2 Hz, 1H), 8.09 (d, J=7.9 Hz, 1H), 8.03-7.98 (m, 2H), 7.90 (dd, J=8.6, 3.4 Hz, 1H), 7.70 (td, J=7.7, 2.3 Hz, 1H), 7.65 (br d, J=7.3 Hz, 1H), 7.58-7.54 (m, 1H), 7.51 (br d, J=7.0 Hz, 1H), 7.36-7.31 (m, 1H), 7.09 (d, J=37.7 Hz, 1H), 5.04-4.85 (m, 1H), 4.45-4.18 (m, 5H), 4.20-3.81 (m, 2H), 3.68-3.59 (m, 2H), 3.15-3.07 (m, 2H), 3.03-2.96 (m, 1H), 2.72-2.62 (m, 1H), 2.41 (br s, 3H), 2.31-2.21 (m, 1H), 2.02-1.93 (m, 1H), 1.74-1.61 (m, 3H).

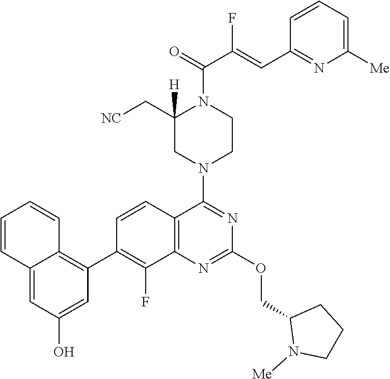 [SMILES: Cc1cccc(/C=C(\F)C(=O)N2CCN(c3nc(OC[C@@H]4CCCN4C)nc4c(F)c(-c5cc(O)cc6ccccc56)ccc34)C[C@@H]2CC#N)n1]

### Example 34

### 2-((S)-1-((Z)-2-fluoro-3-(6-methylpyridin-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile

2-((S)-4-(8-Fluoro-7-(3-hydroxynaphthalen-1-yl)-2-(((S)-1-methylpyrrolidin-2-yl)methoxy)quinazolin-4-yl)piperazin-2-yl)acetonitrile (Intermediate 4, 15 mg, 0.028 mmol) and (Z)-2-fluoro-3-(6-methylpyridin-2-yl)acrylic acid (Intermediate 36, 7 mg, 0.028 mmol) were combined as solids and dissolved in DMF (1 mL). 1-Methylimidazole (23 μL, 0.28 mmol) was added followed by chloro-N,N,N′,N′-tetramethylformamidinium hexafluorophosphate (7.6 mg, 0.028 mmol). The reaction mixture was stirred at room temperature for 5 min. A saturated aqueous solution of ammonium hydroxide (100 μL) was added and the reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 93% 5:95 MeCN:H₂O with 0.1% TFA/7% 95:5 MeCN:H₂O with 0.1% TFA→100% 95:5 MeCN:H₂O with 0.1% TFA; λ=220 nm) to provide the desired product as the TFA salt (11.8 mg, 0.015 mmol, 53% yield); LC/MS (ESI) m/z: [M+H]⁺ calcd for C₃₉H₃₈F₂N₇O₃ 690.3. found 690.4; NMR: ¹H NMR (500 MHz, DMSO-d₆) δ 10.04 (br s, 1H), 9.79 (br s, 1H), 8.04 (d, J=8.2 Hz, 1H), 7.85-7.79 (m, 2H), 7.64 (d, J=7.8 Hz, 1H), 7.49-7.44 (m, 1H), 7.43-7.36 (m, 2H), 7.30-7.24 (m, 3H), 7.13-7.10 (m, 1H), 6.61 (d, J=38.8 Hz, 1H), 5.09-4.88 (m, 1H), 4.77 (br dd, J=12.7, 3.2 Hz, 1H), 4.63 (br dd, J=12.7, 7.0 Hz, 1H), 4.47-4.36 (m, 2H), 3.89-3.81 (m, 1H), 3.66-3.58 (m, 2H), 3.33-3.27 (m, 3H), 3.20-3.08 (m, 6H), 2.98 (br s, 3H), 2.32-2.24 (m, 1H), 2.12-2.05 (m, 1H), 1.98-1.87 (m, 2H)

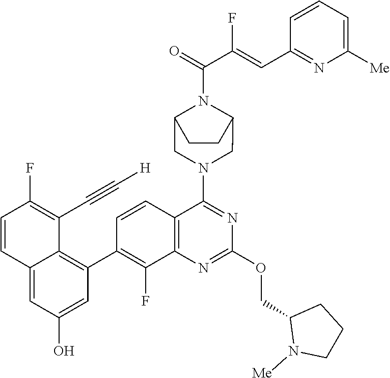 [SMILES: C#Cc1c(F)ccc2cc(O)cc(-c3ccc4c(N5CC6CCC(C5)N6C(=O)/C(F)=C/c5cccc(C)n5)nc(OC[C@@H]5CCCN5C)nc4c3F)c12]

### Example 35

### (Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(6-methylpyridin-2-yl)prop-2-en-1-one

4-(4-((1R,5S)-3,8-Diazabicyclo[3.2.1]octan-3-yl)-8-fluoro-2-(((S)-1-methylpyrrolidin-2-yl)methoxy)quinazolin-7-yl)-5-ethynyl-6-fluoronaphthalen-2-ol (Intermediate 11, 15 mg, 0.027 mmol) and (Z)-2-fluoro-3-(6-methylpyridin-2-yl)acrylic acid (Intermediate 36, 7.0 mg, 0.027 mmol) were combined as solids and dissolved in DMF (1 mL) 1-methylimidazole (22 μL, 0.27 mmol) was added followed by chloro-N,N,N′,N′-tetramethylformamidinium hexafluorophosphate (7.6 mg, 0.027 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (10.0 mg, 0.014 mmol, 50% yield). LC/MS (ESI) m/z: [M+H]⁺ calcd for C₄₁H₃₈F₃N₆O₃ 719.3. found 719.3; NMR: ¹H NMR (500 MHz, DMSO-d₆) δ 10.15 (s, 1H), 7.96 (dd, J=9.3, 6.0 Hz, 1H), 7.81-7.76 (m, 2H), 7.62 (d, J=7.8 Hz, 1H), 7.46 (t, J=9.2 Hz, 1H), 7.36 (d, J=2.2 Hz, 1H), 7.27-7.22 (m, 2H), 7.07 (d, J=2.2 Hz, 1H), 6.76 (d, J=38.1 Hz, 1H), 4.82-4.76 (m, 2H), 4.55-4.48 (m, 1H), 4.47-4.40 (m, 2H), 4.38-4.23 (m, 1H), 4.14-4.06 (m, 1H), 3.85 (s, 1H), 3.71-3.63 (m, 2H), 3.17 (d, J=5.0 Hz, 3H), 2.49 (br s, 3H), 2.09-1.91 (m, 4H), 1.90-1.87 (m, 1H), 1.84-1.64 (m, 3H).

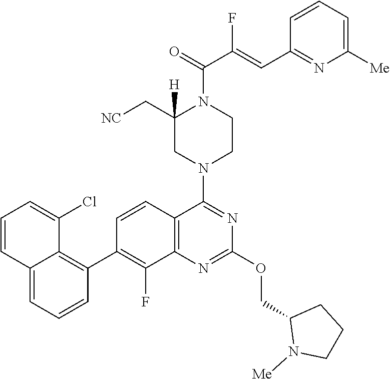 [SMILES: Cc1cccc(/C=C(\F)C(=O)N2CCN(c3nc(OC[C@@H]4CCCN4C)nc4c(F)c(-c5cccc6cccc(Cl)c56)ccc34)C[C@@H]2CC#N)n1]

### Example 36

### 2-((S)-1-((Z)-2-fluoro-3-(6-methylpyridin-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile

2-((S)-4-(8-fluoro-7-(8-chloronaphthyl)-2-(((S)-1-methylpyrrolidin-2-yl)methoxy)quinazolin-4-yl)piperazin-2-yl)acetonitrile (Intermediate 5, 20 mg, 0.037 mmol) and (Z)-2-fluoro-3-(6-methylpyridin-2-yl)acrylic acid (Intermediate 36, 9.5 mg, 0.037 mmol) were combined as solids and dissolved in DMF (1 mL) 1-Methylimidazole (29 μL, 0.37 mmol) was added followed by chloro-N,N,N′,N′-tetramethylformamidinium hexafluorophosphate (10.3 mg, 0.037 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 70% 5:95 MeCN:H₂O with 10 mM AA/30% 95:5 MeCN:H₂O with 10 mM AA→100% 95:5 MeCN:H₂O with 10 mM AA; λ=220 nm) to provide the desired product (6.7 mg, 0.009 mmol, 25% yield). LC/MS (ESI) m/z: [M+H]⁺ calcd for C₃₉H₃₇ClF₂N₇O₂ 708.3. found 708.3; ¹H NMR (500 MHz, DMSO-d₆) δ 8.19-8.15 (m, 1H), 8.11-8.07 (m, 1H), 7.92-7.88 (m, 1H), 7.80-7.76 (m, 1H), 7.73-7.68 (m, 1H), 7.67-7.63 (m, 1H), 7.63-7.59 (m, 1H), 7.59-7.54 (m, 1H), 7.53-7.50 (m, 1H), 7.37-7.31 (m, 1H), 7.28-7.22 (m, 1H), 6.58 (d, J=40.1 Hz, 1H), 5.06-4.82 (m, 1H), 4.38 (dt, J=10.8, 5.1 Hz, 1H), 4.35-4.27 (m, 2H), 4.23-4.18 (m, 1H), 3.63-3.57 (m, 2H), 3.15-3.08 (m, 2H), 2.98-2.89 (m, 2H), 2.61-2.56 (m, 1H), 2.49 (br s, 3H), 2.35 (d, J=2.4 Hz, 4H), 2.20-2.13 (m, 1H), 1.99-1.91 (m, 1H), 1.73-1.55 (m, 4H).

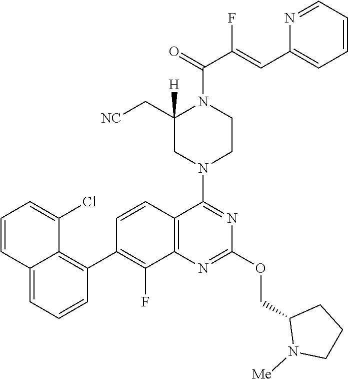 [SMILES: CN1CCC[C@H]1COc1nc(N2CCN(C(=O)/C(F)=C/c3ccccn3)[C@@H](CC#N)C2)c2ccc(-c3cccc4cccc(Cl)c34)c(F)c2n1]

### Example 37

### 2-((S)-1-((Z)-2-fluoro-3-(pyridin-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile

2-((S)-4-(8-fluoro-7-(8-chloronaphthalen-1-yl)-2-(((S)-1-methylpyrrolidin-2-yl)methoxy)quinazolin-4-yl)piperazin-2-yl)acetonitrile (Intermediate 5, 20 mg, 0.037 mmol) and (Z)-2-fluoro-3-(pyridin-2-yl)acrylic acid (Intermediate 27, 6 mg, 0.037 mmol) were combined as solids and dissolved in DMF (1 mL). 1-methylimidazole (23 μL, 0.37 mmol) was added followed by chloro-N,N,N′,N′-tetramethylformamidinium hexafluorophosphate (10.3 mg, 0.037 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (21.9 mg, 0.009 mmol, 30% yield). LC/MS (ESI) m/z: [M+H]⁺ calcd for C₃₈H₃₅ClF₂N₇O₂ 694.3. found 694.2; ¹H NMR (500 MHz, DMSO-d₆) δ 8.65 (br d, J=4.2 Hz, 1H), 8.19-8.15 (m, 1H), 8.11-8.08 (m, 1H), 7.93-7.87 (m, 2H), 7.82-7.78 (m, 1H), 7.73-7.67 (m, 1H), 7.67-7.63 (m, 1H), 7.59-7.54 (m, 1H), 7.54-7.50 (m, 1H), 7.41-7.36 (m, 1H), 7.36-7.30 (m, 1H), 6.65 (d, J=39.0 Hz, 1H), 5.09-4.79 (m, 1H), 4.43-4.28 (m, 3H), 4.24-4.18 (m, 1H), 4.14-4.04 (m, 2H), 3.67-3.58 (m, 1H), 3.20-3.07 (m, 6H), 2.95 (br s, 1H), 2.64-2.56 (m, 1H), 2.36 (br d, J=2.3 Hz, 3H), 2.23-2.14 (m, 1H), 1.99-1.92 (m, 1H), 1.74-1.58 (m, 3H).

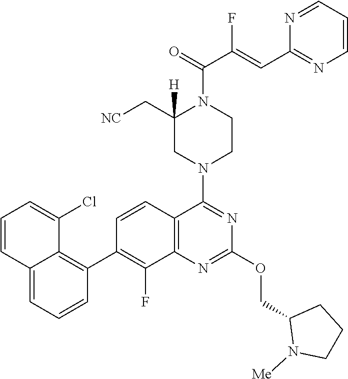 [SMILES: CN1CCC[C@H]1COc1nc(N2CCN(C(=O)/C(F)=C/c3ncccn3)[C@@H](CC#N)C2)c2ccc(-c3cccc4cccc(Cl)c34)c(F)c2n1]

### Example 38

### 2-((S)-1-((Z)-2-fluoro-3-(pyrimidin-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile

2-((S)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile (Intermediate 4, 15 mg, 0.028 mmol) and (Z)-2-fluoro-3-(pyrimidin-2-yl)acrylic acid (Intermediate 26, 4.6 mg, 0.028 mmol) were combined as solids and dissolved in DMF (1 mL). 1-Methylimidazole (23 μL, 0.28 mmol) was added followed by chloro-N,N,N′,N′-tetramethylformamidinium hexafluorophosphate (7.6 mg, 0.028 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 83% 5:95 MeCN:H₂O with 0.1% TFA/17% 95:5 MeCN:H₂O with 0.1% TFA→100% 95:5 MeCN:H₂O with 0.1% TFA; λ=220 nm) to provide the desired product as the TFA salt (10.7 mg, 0.013 mmol, 46% yield) LC/MS (ESI) m/z: [M+H]⁺ calcd for C₃₇H₃₄ClF₂N₈O₂ 695.2. found 695.2; ¹H NMR (500 MHz, DMSO-d₆) δ 8.90 (d, J=4.8 Hz, 2H), 8.20-8.16 (m, 1H), 8.12-8.08 (m, 1H), 7.98-7.91 (m, 1H), 7.74-7.69 (m, 1H), 7.68-7.63 (m, 1H), 7.61-7.54 (m, 1H), 7.54-7.49 (m, 1H), 7.48-7.43 (m, 1H), 7.43-7.36 (m, 1H), 6.63-6.63 (m, 1H), 6.62 (d, J=35.7 Hz, 1H), 5.05-4.87 (m, 1H), 4.77-4.71 (m, 1H), 4.63-4.55 (m, 1H), 4.45-4.33 (m, 2H), 3.87-3.79 (m, 1H), 3.17-3.09 (m, 3H), 2.97 (br s, 3H), 2.31-2.22 (m, 1H), 2.13-2.01 (m, 1H), 1.99-1.81 (m, 2H).

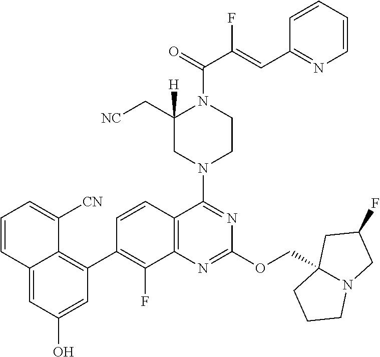 [SMILES: N#CC[C@H]1CN(c2nc(OC[C@@]34CCCN3C[C@H](F)C4)nc3c(F)c(-c4cc(O)cc5cccc(C#N)c45)ccc23)CCN1C(=O)/C(F)=C/c1ccccn1]

### Example 39

### 2-((S)-4-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-1-((Z)-2-fluoro-3-(pyridin-2-yl)acryloyl)piperazin-2-yl)acetonitrile

2-((S)-4-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)piperazin-2-yl)acetonitrile (Intermediate 28, 14 mg, 0.023 mmol) and (Z)-2-fluoro-3-(pyridin-2-yl)acrylic acid (Intermediate 27, 3.9 mg, 0.023 mmol) were combined as solids and dissolved in DMF (1 mL). 1-Methylimidazole (18 μL, 0.23 mmol) was added followed by chloro-N,N,N′,N′-tetramethylformamidinium hexafluorophosphate (6.5 mg, 0.023 mmol) The reaction mixture was stirred at room temperature for 5 min. A saturated aqueous solution of ammonium hydroxide (100 L) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 89% 5:95 MeCN:H₂O with 0.1% TFA/11% 95:5 MeCN:H₂O with 0.1% TFA→100% 95:5 MeCN:H₂O with 0.1% TFA; λ=220 nm) to provide the desired product as the TFA salt (10.4 mg, 0.012 mmol, 54% yield) LC/MS (ESI) m/z: [M+H]⁺ calcd for C₄₀H₃₇F₃N₇O₃ 720.3. found 720.3; ¹H NMR (500 MHz, DMSO-d₆) δ 10.87 (br s, 1H), 8.66 (br d, J=4.2 Hz, 1H), 8.05-8.01 (m, 1H), 7.95-7.89 (m, 1H), 7.84-7.79 (m, 2H), 7.47-7.43 (m, 1H), 7.43-7.34 (m, 3H), 7.29-7.27 (m, 1H), 7.27-7.22 (m, 1H), 7.11 (br s, 1H), 6.67 (d, J=37.9 Hz, 1H), 5.66-5.49 (m, 1H), 5.05-4.81 (m, 1H), 4.69-4.56 (m, 2H), 4.50-4.38 (m, 2H), 4.25-4.10 (m, 1H), 3.94-3.80 (m, 3H), 3.34-3.26 (m, 2H), 3.16-3.09 (m, 1H), 2.65-2.56 (m, 1H), 2.38-2.30 (m, 1H), 2.24-2.13 (m, 2H), 2.11-2.01 (m, 1H)).

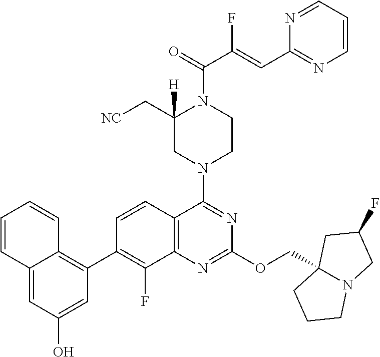 [SMILES: N#CC[C@H]1CN(c2nc(OC[C@@]34CCCN3C[C@H](F)C4)nc3c(F)c(-c4cc(O)cc5ccccc45)ccc23)CCN1C(=O)/C(F)=C/c1ncccn1]

### Example 40

### 2-((S)-4-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-1-((Z)-2-fluoro-3-(pyrimidin-2-yl)acryloyl)piperazin-2-yl)acetonitrile

2-((S)-4-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)piperazin-2-yl)acetonitrile (Intermediate 28, 14 mg, 0.023 mmol) and (Z)-2-fluoro-3-(pyrimidin-2-yl)acrylic acid (Intermediate 26, 3.9 mg, 0.023 mmol) were combined as solids and dissolved in DMF (1 mL). 1-Methylimidazole (18 μL, 0.23 mmol) was added followed by chloro-N,N,N′,N′-tetramethylformamidinium hexafluorophosphate (6.5 mg, 0.023 mmol). The reaction mixture was stirred at room temperature for 5 min. A saturated aqueous solution of ammonium hydroxide (100 L) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 88% 5:95 MeCN:H₂O with 0.1% TFA/12% 95:5 MeCN:H₂O with 0.1% TFA→100% 95:5 MeCN:H₂O with 0.1% TFA; λ=220 nm) to provide the desired product as the TFA salt (7.4 mg, 0.010 mmol, 44% yield) LC/MS (ESI) m/z: [M+H]⁺ calcd for C₃₉H₃₆F₃N₈O₃ 721.3. found 721.3; ¹H NMR (500 MHz, DMSO-d₆) δ 10.86 (br s, 1H), 8.90 (br d, J=4.6 Hz, 2H), 8.05-8.01 (m, 1H), 7.83-7.79 (m, 1H), 7.47-7.43 (m, 2H), 7.42-7.33 (m, 2H), 7.29-7.27 (m, 1H), 7.27-7.22 (m, 1H), 7.11 (br s, 1H), 6.63 (d, J=35.3 Hz, 1H), 5.65-5.47 (m, 1H), 5.09-4.79 (m, 1H), 4.66-4.56 (m, 2H), 4.51-4.39 (m, 2H), 3.93-3.74 (m, 4H), 3.35-3.25 (m, 3H), 3.18-3.12 (m, 1H), 2.64-2.58 (m, 1H), 2.39-2.30 (m, 1H), 2.26-2.12 (m, 2H), 2.12-2.02 (m, 1H).

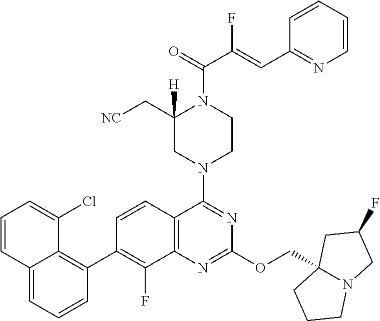 [SMILES: N#CC[C@H]1CN(c2nc(OC[C@@]34CCCN3C[C@H](F)C4)nc3c(F)c(-c4cccc5cccc(Cl)c45)ccc23)CCN1C(=O)/C(F)=C/c1ccccn1]

### Example 41

### 2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyridin-2-yl)acryloyl)piperazin-2-yl)acetonitrile

2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile (Intermediate 29, 12 mg, 0.019 mmol) and (Z)-2-fluoro-3-(pyridin-2-yl)acrylic acid (Intermediate 27, 3.2 mg, 0.019 mmol) were combined as solids and dissolved in DMF (1 mL). 1-Methylimidazole (15 μL, 0.19 mmol) was added followed by chloro-N,N,N′,N′-tetramethylformamidinium hexafluorophosphate (5.4 mg, 0.019 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 67% 5:95 MeCN:H₂O with 10 mM AA/33% 95:5 MeCN:H₂O with 10 mM AA→100% 95:5 MeCN:H₂O with 10 nM AA; λ=220 nm) to provide the desired product (1.4 mg, 0.002 mmol, 9% yield) LC/MS (ESI) m/z: [M+H]⁺ calcd for C₄₀H₃₆ClF₃N₇O₃ 738.3. found 738.1.

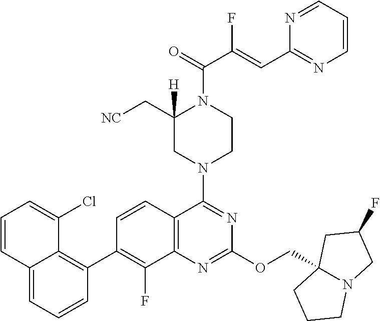 [SMILES: N#CC[C@H]1CN(c2nc(OC[C@@]34CCCN3C[C@H](F)C4)nc3c(F)c(-c4cccc5cccc(Cl)c45)ccc23)CCN1C(=O)/C(F)=C/c1ncccn1]

### Example 42

### 2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyrimidin-2-yl)acryloyl)piperazin-2-yl)acetonitrile

2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile (Intermediate 29, 12 mg, 0.019 mmol) and (Z)-2-fluoro-3-(pyrimidin-2-yl)acrylic acid (Intermediate 26, 3.2 mg, 0.019 mmol) were combined as solids and dissolved in DMF (1 mL). 1-Methylimidazole (15 μL, 0.19 mmol) was added followed by chloro-N,N,N′,N′-tetramethylformamidinium hexafluorophosphate (5.4 mg, 0.019 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 81% 5:95 MeCN:H₂O with 0.1% TFA/19% 95:5 MeCN:H₂O with 0.1% TFA→100% 95:5 MeCN:H₂O with 0.1% TFA; λ=220 nm) to provide the desired product as the TFA salt (2.7 mg, 0.004 mmol, 19% yield) LC/MS (ESI) m/z: [M+H]⁺ calcd for C₃₉H₃₇ClF₃N₈O₃ 739.3. found 739.2; ¹H NMR (500 MHz, DMSO-d₆) δ 8.90 (d, J=5.0 Hz, 2H), 8.21-8.16 (m, 1H), 8.12-8.09 (m, 1H), 7.98-7.93 (m, 1H), 7.74-7.69 (m, 1H), 7.68-7.63 (m, 1H), 7.57 (t, J=7.6 Hz, 1H), 7.54-7.50 (m, 1H), 7.46 (t, J=4.9 Hz, 1H), 7.43-7.36 (m, 1H), 6.63 (d, J=34.4 Hz, 1H), 5.63-5.48 (m, 1H), 5.10-4.73 (m, 1H), 4.68-4.52 (m, 2H), 4.49-4.34 (m, 2H), 3.89-3.83 (m, 1H), 3.81-3.73 (m, 2H), 2.66 (br d, J=2.3 Hz, 1H), 2.41-2.30 (m, 1H), 2.23-2.13 (m, 2H), 2.08-2.00 (m, 1H).

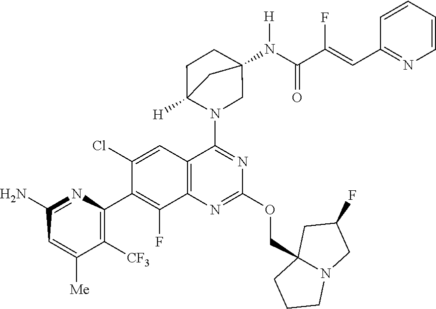

### Example 43

### (2Z)-N-[(1S,4S)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-4-yl]-2-fluoro-3-(pyridin-2-yl)prop-2-enamide

(1S,4S)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-4-amine (Intermediate 30, 3 mg, 0.005 mmol) and (Z)-2-fluoro-3-(pyridin-2-yl)acrylic acid (Intermediate 27, 2.7 mg, 0.016 mmol) were combined as solids and dissolved in DMF (300 μL). 1-methylimidazole (4 μL, 0.053 mmol) was added followed by chloro-N,N,N′,N′-tetramethylformamidinium hexafluorophosphate (4.5 mg, 0.016 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (2.2 mg, 0.002 mmol, 54% yield). LC/MS (ESI) m/z: [M+H]⁺ calcd for C₃₇H₃₆ClF₆N₈O₃ 773.3. found 773.3; ¹H NMR (500 MHz, DMSO-d₆) δ 9.22 (br s, 1H), 8.65 (br d, J=4.1 Hz, 1H), 7.92 (br s, 1H), 7.89 (br t, J=8.1 Hz, 1H), 7.78 (d, J=8.0 Hz, 1H), 7.41-7.35 (m, 1H), 6.94 (d, J=37.2 Hz, 1H), 6.82 (d, J=8.0 Hz, 1H), 6.83 (br s, 1H), 6.49 (s, 1H), 5.42-5.26 (m, 1H), 4.90 (br s, 1H), 4.41-4.21 (m, 1H), 4.14 (br d, J=10.5 Hz, 1H), 4.03 (br d, J=10.5 Hz, 1H), 3.30-3.23 (m, 2H), 2.97-2.91 (m, 1H), 2.87-2.82 (m, 1H), 2.79-2.74 (m, 1H), 2.58-2.52 (m, 1H), 2.36 (br s, 3H), 2.34-2.24 (m, 1H), 2.18 (br d, J=9.3 Hz, 1H), 2.09 (br d, J=9.2 Hz, 1H), 2.04-1.91 (m, 4H), 1.90-1.87 (m, 1H), 1.85-1.79 (m, 2H), 1.69-1.62 (m, 1H).

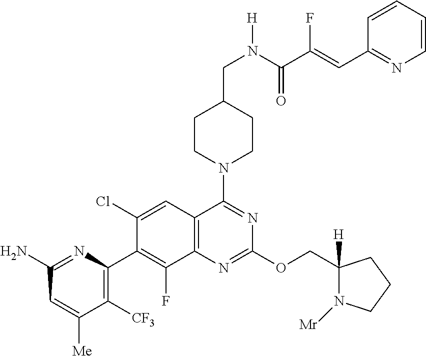

### Example 4

### (2Z)-N-({1-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]piperidin-4-yl}methyl)-2-fluoro-3-(pyridin-2-yl)prop-2-enamide

(5M)-6-{4-[4-(aminomethyl)piperidin-1-yl]-6-chloro-8-fluoro-2-{[(2S)-1-methylpyrrolidin-2-yl]methoxy}quinazolin-7-yl}-4-methyl-5-(trifluoromethyl)pyridin-2-amine (prepared in a way similar to the other analogs in this position (i.e., BOP coupling of amine followed by deprotection) (3 mg, 0.005 mmol) and (Z)-2-fluoro-3-(pyridin-2-yl)acrylic acid (Intermediate 27, 2.5 mg, 0.015 mmol) were combined as solids and dissolved in DMF (300 μL) 1-Methylimidazole (4 μL, 0.05 mmol) was added followed by chloro-N,N,N′,N′-tetramethylformamidinium hexafluorophosphate (4.3 mg, 0.015 mmol) The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (0.8 mg, 0.001 mmol, 21% yield); LC/MS (ESI) m/z: [M+H]⁺ calcd for C₃₅H₃₇ClF₅N₈O₃ 731.3; found 731.2; ¹H NMR (500 MHz, CDCl₃) δ 8.70-8.66 (m, 1H), 7.74-7.72 (m, 2H), 7.64 (d, J=1.3 Hz, 1H), 7.25-7.22 (m, 1H), 7.15 (d, J=38.3 Hz, 1H), 6.61-6.56 (m, 1H), 6.47 (s, 1H), 4.78 (s, 2H), 4.67-4.56 (m, 1H), 4.44-4.29 (m, 3H), 3.45-3.39 (m, 2H), 3.29-3.09 (m, 3H), 2.62-2.54 (m, 2H), 2.48 (d, J=1.4 Hz, 3H), 2.16-2.08 (m, 1H), 2.05-1.97 (m, 1H), 1.96-1.72 (m, 6H), 1.55-1.46 (m, 3H).

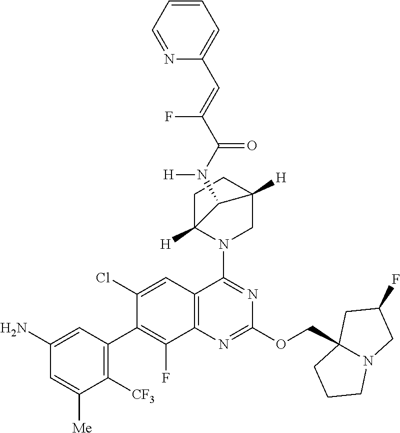 [SMILES: Cc1cc(N)cc(-c2c(Cl)cc3c(N4C[C@H]5CC[C@@H]4[C@@H]5NC(=O)/C(F)=C/c4ccccn4)nc(OC[C@]45CCCN4C[C@H](F)C5)nc3c2F)c1C(F)(F)F]

### Example 45

### (2Z)-N-[(1R,4R,7R)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-7-yl]-2-fluoro-3-(pyridin-2-yl)prop-2-enamide

(1R,4R,7R)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-7-amine (Intermediate 31, 3.7 mg, 0.006 mmol) and (Z)-2-fluoro-3-(pyridin-2-yl)acrylic acid (Intermediate 27, 2.9 mg, 0.018 mmol) were combined as solids and dissolved in DMF (300 μL) 1-methylimidazole (5 μL, 0.06 mmol) was added followed by chloro-N,N,N′,N′-tetramethylformamidinium hexafluorophosphate (5.0 mg, 0.018 mmol) The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (2.4 mg, 0.003 mmol, 52% yield). LC/MS (ESI) m/z: [M+H]⁺ calcd for C₃₇H₃₆ClF₆N₈O₃ 773.3. found 773.2; ¹H NMR (500 MHz, DMSO-d₆) δ 8.79 (br s, 1H), 8.66 (br d, J=4.5 Hz, 1H), 7.90 (br td, J=7.7, 1.7 Hz, 1H), 7.79 (d, J=8.2 Hz, 1H), 7.38 (ddd, J=7.5, 4.9, 1.0 Hz, 1H), 6.94 (d, J=36.9 Hz, 1H), 6.82 (br s, 2H), 6.49 (s, 1H), 5.43-5.26 (m, 1H), 5.11 (br s, 1H), 4.17-4.03 (m, 3H), 3.31-3.22 (m, 2H), 3.19-3.15 (m, 2H), 2.98-2.91 (m, 1H), 2.90-2.88 (m, 1H), 2.87-2.75 (m, 1H), 2.59-2.54 (m, 1H), 2.36 (br s, 3H), 2.35-2.26 (m, 1H), 2.04-1.94 (m, 2H), 1.94-1.87 (m, 3H), 1.86-1.76 (m, 3H), 1.70-1.62 (m, 1H), 1.61-1.51 (m, 1H).

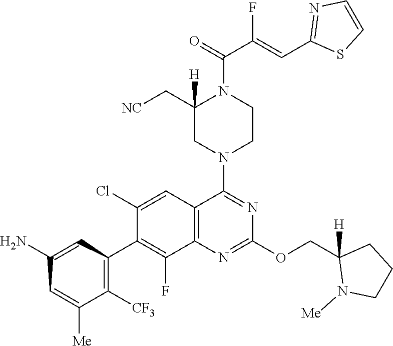

### Example 46

### 2-[(2S)-4-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-1-[(2Z)-2-fluoro-3-(thiazol-2-yl)prop-2-enoyl]piperazin-2-yl]acetonitrile

2-[(2S)-4-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]piperazin-2-yl]acetonitrile (8 mg, 0.013 mmol) and (Z)-2-fluoro-3-(thiazol-2-yl)acrylic acid (7 mg, 0.04 mmol) were combined as solids and dissolved in DMF (600 μL). 1-Methylimidazole (6 μL, 0.08 mmol) was added followed by chloro-N,N,N′,N′-tetramethylformamidinium hexafluorophosphate (11.4 mg, 0.04 mmol) The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 78% 5:95 MeCN:H₂O with 10 mM AA/22% 95:5 MeCN:H₂O with 10 mM AA→100% 95:5 MeCN:H₂O with 10 mM AA; λ=220 nm) to provide the desired product (2.1 mg, 0.003 mmol, 21% yield) LC/MS (ESI) m/z: [M+H]⁺ calcd for C₃₃H₃₂ClF₅N₉O₂S 748.2. found 748.2; ¹H NMR (500 MHz, DMSO-d₆) δ 8.04-7.94 (m, 3H), 7.08 (d, J=37.2 Hz, 1H), 6.84 (br s, 2H), 6.50 (s, 1H), 5.01-4.82 (m, 1H), 4.38 (dd, J=10.7, 4.5 Hz, 1H), 4.29-4.23 (m, 2H), 4.18 (dd, J=10.7, 6.3 Hz, 1H), 3.69-3.60 (m, 1H), 3.11-3.05 (m, 1H), 2.98-2.91 (m, 1H), 2.60-2.55 (m, 1H), 2.37 (br s, 2H), 2.36 (s, 3H), 2.21-2.14 (m, 1H), 1.99-1.91 (m, 1H), 1.72-1.58 (m, 3H).

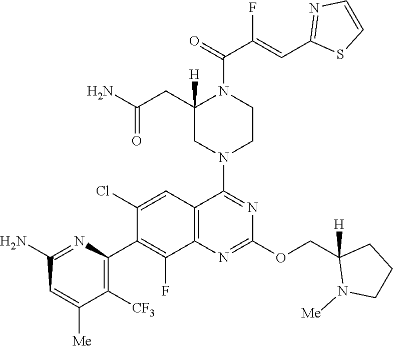

### Example 47

### 2-[(2S)-4-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-1-[(2Z)-2-fluoro-3-(thiazol-2-yl)prop-2-enoyl]piperazin-2-yl]acetamide

2-[(2S)-4-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]piperazin-2-yl]acetamide (Intermediate 33B, 4 mg, 0.06 mmol) and (Z)-2-fluoro-3-(thiazol-2-yl)acrylic acid (Intermediate 25, 2.3 mg, 0.013 mmol) were combined as solids and dissolved in DMF (600 μL). 1-Methylimidazole (3 μL, 0.03 mmol) was added followed by chloro-N,N,N′,N′-tetramethylformamidinium hexafluorophosphate (3.7 mg, 0.013 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 83% 5:95 MeCN:H₂O with 10 mM AA/17% 95:5 MeCN:H₂O with 10 mM AA→100% 95:5 MeCN:H₂O with 10 mM AA; λ=220 nm) to provide the desired product (1.9 mg, 0.002 mmol, 38% yield) LC/MS (ESI) m/z: [M+H]⁺ calcd for C₃₃H₃₄ClF₅N₉O₃S 766.2; found 766.2; ¹H NMR (500 MHz, DMSO-d₆) δ 8.02-7.96 (m, 2H), 7.91 (s, 1H), 7.43 (br s, 1H), 7.04 (d, J=37.6 Hz, 1H), 6.93 (br s, 1H), 6.85 (s, 2H), 6.50 (s, 1H), 4.92-4.75 (m, 1H), 4.46-4.39 (m, 1H), 4.35-4.21 (m, 3H), 4.18-4.00 (m, 1H), 3.71-3.62 (m, 1H), 3.09-2.99 (m, 1H), 2.72-2.61 (m, 2H), 2.45 (br s, 3H), 2.37 (br d, J=1.3 Hz, 3H), 2.04-1.95 (m, 1H), 1.77-1.63 (m, 3H).

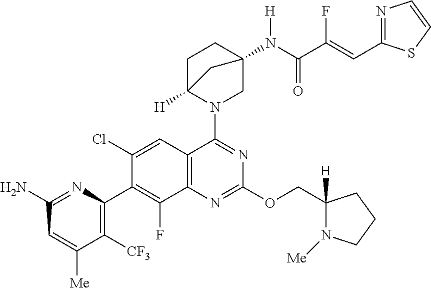

### Example 48

### (2Z)-N-[(1S,4S)-2-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-2-azabicyclo[2.2.1]heptan-4-yl]-2-fluoro-3-(thiazol-2-yl)prop-2-enamide

(1S,4S)-2-[(7M)-7-[6-Amino-4-methyl-3-(trifluoromethyl)pyridin-2-yl]-6-chloro-8-fluoro-2-{[(2S)-1-methylpyrrolidin-2-yl]methoxy}quinazolin-4-yl]-2-azabicyclo[2.2.1]heptan-4-amine (Intermediate 34, 40 mg, 0.069 mmol) and (Z)-2-fluoro-3-(thiazol-2-yl)acrylic acid (Intermediate 25, 24 mg, 0.14 mmol) were combined as solids and dissolved in DMF (700 μL). 1-Methylimidazole (28 μL, 0.35 mmol) was added followed by chloro-N,N,N′,N′-tetramethylformamidinium hexafluorophosphate (39 mg, 0.14 mmol). The reaction mixture was stirred at room temperature for 5 min. The reaction mixture was directly purified by reverse phase HPLC (column: Xbridge C18, 19 mm×200 mm, 5 μm particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (5.6 mg, 0.007 mmol, 11% yield) LC/MS (ESI) m/z: [M+H]⁺ calcd for C₃₃H₃₃ClF₅N₈O₂S 735.2; found 735.2; ¹H NMR (500 MHz, DMSO-d₆) δ 9.31 (s, 1H), 8.04-7.99 (m, 2H), 7.93 (br s, 1H), 7.29 (d, J=36.2 Hz, 1H), 6.85 (s, 2H), 6.49 (s, 1H), 4.90 (br s, 1H), 4.41 (br dd, J=10.6, 4.8 Hz, 1H), 4.38-4.21 (m, 1H), 4.17-4.03 (m, 2H), 2.98-2.92 (m, 1H), 2.61-2.55 (m, 1H), 2.37 (d, J=1.5 Hz, 3H), 2.36 (s, 3H), 2.21-2.14 (m, 2H), 2.13-2.09 (m, 1H), 2.05-1.91 (m, 5H), 1.72-1.58 (m, 3H).

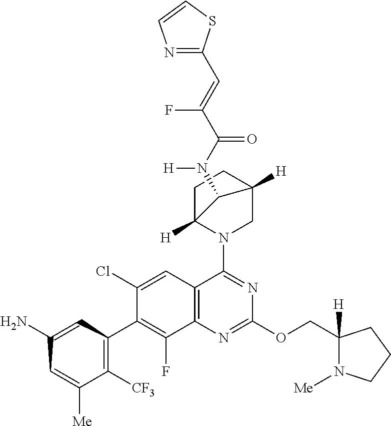

### Example 49

### (2Z)-N-[(1R,4R,7R)-2-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-2-azabicyclo[2.2.1]heptan-7-yl]-2-fluoro-3-(1,3-thiazol-2-yl)prop-2-enamide

(1R,4R,7R)-2-[(7M)-7-[6-Amino-4-methyl-3-(trifluoromethyl)pyridin-2-yl]-6-chloro-8-fluoro-2-{[(2S)-1-methylpyrrolidin-2-yl]methoxy}quinazolin-4-yl]-2-azabicyclo[2.2.1]heptan-7-amine (Intermediate 35, 40 mg, 0.069 mmol) and (Z)-2-fluoro-3-(thiazol-2-yl)acrylic acid (Intermediate 25, 24 mg, 0.14 mmol) were combined as solids and dissolved in DMF (700 μL). 1-Methylimidazole (28 μL, 0.35 mmol) was added followed by chloro-N,N,N′,N′-tetramethylformamidinium hexafluorophosphate (39 mg, 0.14 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (9.8 mg, 0.013 mmol, 19% yield). LC/MS (ESI) m/z: [M+H]⁺ calcd for C₃₃H₃₃ClF₅N₈O₂S 735.2. found 735.2; ¹H NMR (500 MHz, DMSO-d₆) δ 8.86 (br s, 1H), 8.04-8.00 (m, 2H), 7.94 (br s, 1H), 7.29 (d, J=36.0 Hz, 1H), 6.82 (br s, 2H), 6.49 (s, 1H), 5.16-5.04 (m, 1H), 4.34 (br dd, J=10.7, 4.6 Hz, 1H), 4.15 (dd, J=10.7, 6.5 Hz, 1H), 3.96-3.90 (m, 1H), 2.98-2.93 (m, 1H), 2.86 (br s, 1H), 2.62-2.55 (m, 1H), 2.36 (s, 6H), 2.22-2.14 (m, 1H), 2.05-1.92 (m, 3H), 1.88-1.79 (m, 1H), 1.73-1.51 (m, 4H).

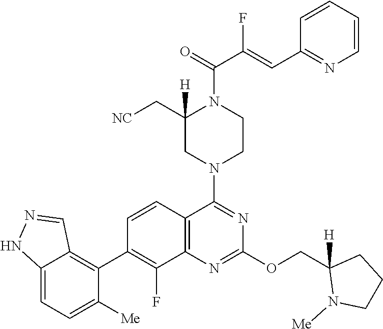 [SMILES: Cc1ccc2[nH]ncc2c1-c1ccc2c(N3CCN(C(=O)/C(F)=C/c4ccccn4)[C@@H](CC#N)C3)nc(OC[C@@H]3CCCN3C)nc2c1F]

### Example 50

### 2-((2S)-1-((Z)-2-fluoro-3-(pyridin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile

2-((2S)-4-(8-Fluoro-7-(5-methyl-1H-indazol-4-yl)-2-(((S)-1-methylpyrrolidin-2-yl)methoxy)quinazolin-4-yl)piperazin-2-yl)acetonitrile (18 mg, 0.035 mmol) and (Z)-2-fluoro-3-(pyridin-2-yl)acrylic acid (5.9 mg, 0.035 mmol) were combined as solids and dissolved in DMF (1.0 mL). 1-Methylimidazole (28 μL, 0.35 mmol) was added followed by chloro-N,N,N′,N′-tetramethylformamidinium hexafluorophosphate (9.8 mg, 0.035 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 86% 5:95 MeCN:H₂O with 10 mM AA/14% 95:5 MeCN:H₂O with 10 mM AA→100% 95:5 MeCN:H₂O with 10 mM AA; λ=220 nm) to provide the desired product (13.3 mg, 0.02 mmol, 54% yield). LC/MS (ESI) m/z: [M+H]⁺ calcd for C₃₆H₃₆F₂N₉O₂ 664.3. found 664.3; ¹H NMR (500 MHz, DMSO-d₆) δ 8.66 (br d, J=4.3 Hz, 1H), 7.99 (br t, J=7.7 Hz, 1H), 7.91 (t, J=7.7 Hz, 1H), 7.81 (d, J=8.0 Hz, 1H), 7.60-7.57 (m, 1H), 7.57 (s, 1H), 7.43-7.31 (m, 3H), 6.69-6.62 (br d, 1H), 4.46-4.25 (m, 4H), 3.63 (br dd, J=5.7, 1.9 Hz, 1H), 3.45 (br s, 2H), 3.12 (dd, J=17.2, 5.9 Hz, 1H), 3.02 (br s, 2H), 2.56-2.53 (m, 2H), 2.49-2.37 (m, 3H), 2.32-2.19 (m, 5H), 2.04-1.91 (m, 3H), 1.79-1.62 (m, 3H).

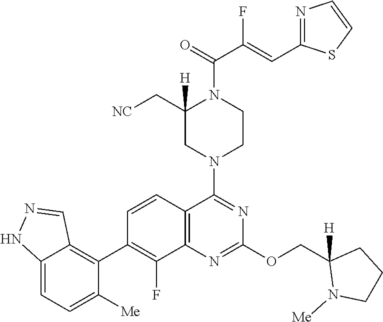 [SMILES: Cc1ccc2[nH]ncc2c1-c1ccc2c(N3CCN(C(=O)/C(F)=C/c4nccs4)[C@@H](CC#N)C3)nc(OC[C@@H]3CCCN3C)nc2c1F]

### Example 51

### 2-((2S)-1-((Z)-2-fluoro-3-(thiazol-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile

2-((2S)-4-(8-Fluoro-7-(5-methyl-1H-indazol-4-yl)-2-(((S)-1-methylpyrrolidin-2-yl)methoxy)quinazolin-4-yl)piperazin-2-yl)acetonitrile (18 mg, 0.035 mmol) and (Z)-2-fluoro-3-(thiazol-2-yl)acrylic acid (6.1 mg, 0.035 mmol) were combined as solids and dissolved in DMF (1.0 mL). 1-Methylimidazole (28 μL, 0.35 mmol) was added followed by chloro-N,N,N′,N′-tetramethylformamidinium hexafluorophosphate (9.8 mg, 0.035 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 86% 5:95 MeCN:H₂O with 10 mM AA/14% 95:5 MeCN:H₂O with 10 mM AA→100% 95:5 MeCN:H₂O with 10 mM AA; λ=220 nm) to provide the desired product (11.6 mg, 0.02 mmol, 47% yield). LC/MS (ESI) m/z: [M+H]⁺ calcd for C₃₄H₃₄F₂N₉O₂S 670.3; found 670.4; ¹H NMR (500 MHz, DMSO-d₆) δ 8.05-7.95 (m, 3H), 7.61-7.54 (m, 2H), 7.42-7.29 (m, 2H), 7.11 (d, J=35.5 Hz, 1H), 5.06-4.83 (m, 1H), 4.46-4.07 (m, 4H), 3.70-3.57 (m, 1H), 3.47-3.37 (m, 1H), 3.15-3.05 (m, 1H), 3.05-2.94 (m, 1H), 2.67-2.52 (m, 2H), 2.39 (s, 3H), 2.28-2.19 (m, 4H), 2.00-1.90 (m, 4H), 1.77-1.62 (m, 3H).

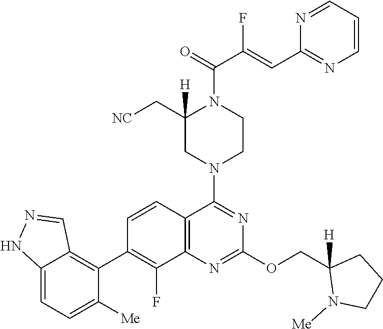 [SMILES: Cc1ccc2[nH]ncc2c1-c1ccc2c(N3CCN(C(=O)/C(F)=C/c4ncccn4)[C@@H](CC#N)C3)nc(OC[C@@H]3CCCN3C)nc2c1F]

### Example 52

### 2-((2S)-1-((Z)-2-fluoro-3-(pyrimidin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile

2-((2S)-4-(8-Fluoro-7-(5-methyl-1H-indazol-4-yl)-2-(((S)-1-methylpyrrolidin-2-yl)methoxy)quinazolin-4-yl)piperazin-2-yl)acetonitrile (14 mg, 0.027 mmol) and (Z)-2-fluoro-3-(pyrimidin-2-yl)acrylic acid (4.5 mg, 0.027 mmol) were combined as solids and dissolved in DMF (1.0 mL). 1-Methylimidazole (22 μL, 0.27 mmol) was added followed by chloro-N,N,N′,N′-tetramethylformamidinium hexafluorophosphate (7.6 mg, 0.027 mmol). The reaction mixture was stirred at room temperature for 5 min. The reaction mixture was directly purified by reverse phase HPLC (column: Xbridge C18, 19 mm×200 mm, 5 μm particles; flow rate: 20 mL/min; column temperature: 25° C.; gradient: 96% 5:95 MeCN:H₂O with 10 mM AA/4% 95:5 MeCN:H₂O with 10 mM AA→100% 95:5 MeCN:H₂O with 10 mM AA; λ=220 nm) to provide the desired product (3 mg, 0.004 mmol, 17% yield). LC/MS (ESI) m/z: [M+H]⁺ calcd for C₃₅H₃₅F₂N₁₀O₂ 665.3. found 665.3; ¹H NMR (500 MHz, CD₃OD) δ 8.89-8.81 (m, 3H), 8.07 (br d, J=11.2 Hz, 1H), 7.71-7.55 (m, 1H), 7.48-7.38 (m, 3H), 6.83 (d, J=37 Hz, 1H), 4.64-4.48 (m, 2H), 4.52-4.44 (m, 1H), 4.15 (t, J=6.6 Hz, 5H), 3.26-3.14 (m, 3H), 3.01-2.95 (m, 3H), 2.88-2.80 (m, 3H), 2.07-1.93 (m, 1H), 1.84-1.61 (m, 4H), 0.93-0.89 (m, 3H).

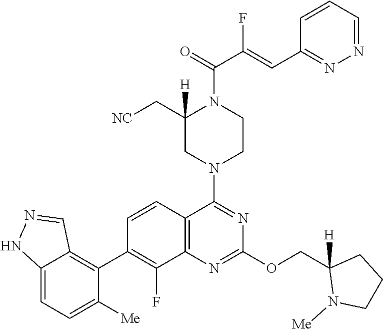 [SMILES: Cc1ccc2[nH]ncc2c1-c1ccc2c(N3CCN(C(=O)/C(F)=C/c4cccnn4)[C@@H](CC#N)C3)nc(OC[C@@H]3CCCN3C)nc2c1F]

### Example 53

### 2-((2S)-1-((Z)-2-fluoro-3-(pyridazin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile

2-((2S)-4-(8-Fluoro-7-(5-methyl-1H-indazol-4-yl)-2-(((S)-1-methylpyrrolidin-2-yl)methoxy)quinazolin-4-yl)piperazin-2-yl)acetonitrile (11 mg, 0.021 mmol) and (Z)-2-fluoro-3-(pyridazin-2-yl)acrylic acid (3.6 mg, 0.021 mmol) were combined as solids and dissolved in DMF (1.0 mL). 1-Methylimidazole (17 μL, 0.21 mmol) was added followed by chloro-N,N,N′,N′-tetramethylformamidinium hexafluorophosphate (6.0 mg, 0.021 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 92% 5:95 MeCN:H₂O with 10 mM AA/8% 95:5 MeCN:H₂O with 10 mM AA→100% 95:5 MeCN:H₂O with 10 mM AA; λ=220 nm) to provide the desired product (4.3 mg, 0.006 mmol, 29% yield). LC/MS (ESI) m/z: [M+H]⁺ calcd for C₃₅H₃₅F₂N₁₀O₂ 665.3; found 665.2; ¹H NMR (500 MHz, DMSO-d₆) δ 9.21 (br d, J=4.0 Hz, 1H), 8.06 (d, J=8.5 Hz, 1H), 7.99 (br t, J=7.6 Hz, 1H), 7.81 (br dd, J=8.2, 5.2 Hz, 1H), 7.61-7.54 (m, 2H), 7.42-7.28 (m, 2H), 6.84 (d, J=37 Hz, 1H), 4.46-4.29 (m, 3H), 4.16 (br s, 1H), 3.90 (s, 1H), 3.01-2.94 (m, 2H), 2.74 (s, 1H), 2.66-2.53 (m, 2H), 2.50-2.34 (m, 4H), 2.25 (s, 3H), 2.18 (q, J=8.6 Hz, 1H), 1.98-1.93 (m, 3H), 1.76 (s, 1H), 1.73-1.60 (m, 3H).

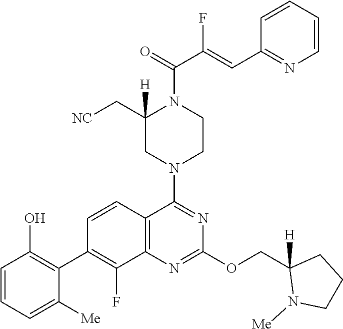 [SMILES: Cc1cccc(O)c1-c1ccc2c(N3CCN(C(=O)/C(F)=C/c4ccccn4)[C@@H](CC#N)C3)nc(OC[C@@H]3CCCN3C)nc2c1F]

### Example 5

### 2-((2S)-1-((Z)-2-fluoro-3-(pyridin-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile

2-((2S)-4-(8-Fluoro-7-(2-fluoro-6-hydroxyphenyl)-2-(((S)-1-methylpyrrolidin-2-yl)methoxy)quinazolin-4-yl)piperazin-2-yl)acetonitrile (15 mg, 0.030 mmol) and (Z)-2-fluoro-3-(pyridin-2-yl)acrylic acid (5.1 mg, 0.030 mmol) were combined as solids and dissolved in DMF (1.0 mL). 1-Methylimidazole (24 μL, 0.30 mmol) was added followed by chloro-N,N,N′,N′-tetramethylformamidinium hexafluorophosphate (8.5 mg, 0.030 mmol). The reaction mixture was stirred at room temperature for 5 min. A saturated aqueous solution of ammonium hydroxide (100 L) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 86% 5:95 MeCN:H₂O with 10 mM AA/14% 95:5 MeCN:H₂O with 10 mM AA→100% 95:5 MeCN:H₂O with 10 mM AA; λ=220 nm) to provide the desired product (7.4 mg, 0.012 mmol, 37% yield). LC/MS (ESI) m/z: [M+H]⁺ calcd for C₃₄H₃₃F₃N₇O₃ 644.3. found 644.3; ¹H NMR (500 MHz, DMSO-d₆) δ 8.00-7.84 (m, 2H), 7.35-7.26 (m, 3H), 6.90-6.77 (m, 3H), 6.65 (d, J=36 Hz, 1H), 4.48-4.42 (m, 4H), 4.35-4.20 (m, 2H), 3.65-3.42 (m, 5H), 3.28-3.23 (m, 1H), 3.32-3.30 (m, 1H), 2.74-2.52 (m, 1H), 2.49-2.34 (m, 3H), 2.07-1.90 (m, 4H), 1.78-1.63 (m, 2H).

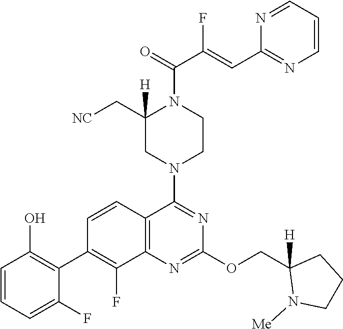 [SMILES: CN1CCC[C@H]1COc1nc(N2CCN(C(=O)/C(F)=C/c3ncccn3)[C@@H](CC#N)C2)c2ccc(-c3c(O)cccc3F)c(F)c2n1]

### Example 55

### 2-((2S)-1-((Z)-2-fluoro-3-(pyrimidin-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile

2-((2S)-4-(8-Fluoro-7-(2-fluoro-6-hydroxyphenyl)-2-(((S)-1-methylpyrrolidin-2-yl)methoxy)quinazolin-4-yl)piperazin-2-yl)acetonitrile (15 mg, 0.030 mmol) and (Z)-2-fluoro-3-(pyrimidin-2-yl)acrylic acid (5.1 mg, 0.030 mmol) were combined as solids and dissolved in DMF (1.0 mL). 1-Methylimidazole (24 μL, 0.30 mmol) was added followed by chloro-N,N,N′,N′-tetramethylformamidinium hexafluorophosphate (8.5 mg, 0.030 mmol). The reaction mixture was stirred at room temperature for 5 min. A saturated aqueous solution of ammonium hydroxide (100 L) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 94% 5:95 MeCN:H₂O with 0.1% TFA/6% 95:5 MeCN:H₂O with 0.1% TFA→100% 95:5 MeCN:H₂O with 0.1% TFA; λ=220 nm) to provide the desired product as the bis TFA salt (16.2 mg, 0.02 mmol, 62% yield). LC/MS (ESI) m/z: [M+H]⁺ calcd for C₃₃H₃₂F₃N₈O₃ 645.3. found 645.1; ¹H NMR (500 MHz, DMSO-d₆) δ 8.90 (d, J=4.8 Hz, 1H), 8.00-7.84 (m, 1H), 7.46 (t, J=1.9 Hz, 1H), 7.35-7.30 (m, 2H) 7.23 (t, J=0.9 Hz, 1H) 7.13 (t, J=0.9 Hz, 1H) 7.03 (t, J=0.9 Hz, 1H) 6.82 (d, J=36 Hz, 1H), 4.88-4.71 (m, 2H), 4.46-4.18 (m, 3H), 3.96 (s, 1H), 3.93-3.79 (m, 1H), 3.72-3.47 (m, 2H), 3.40-3.08 (m, 5H), 3.01-2.95 (m, 1H), 2.55-2.49 (m, 4H), 2.38-2.18 (m, 1H), 2.14-2.01 (m, 1H), 2.01-1.87 (m, 1H).

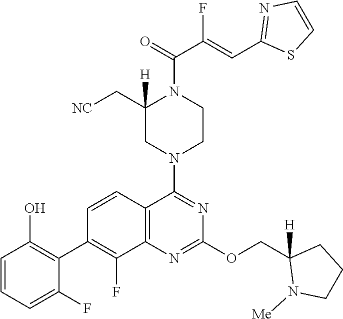 [SMILES: CN1CCC[C@H]1COc1nc(N2CCN(C(=O)/C(F)=C/c3nccs3)[C@@H](CC#N)C2)c2ccc(-c3c(O)cccc3F)c(F)c2n1]

### Example 56

### 2-((2S)-1-((Z)-2-fluoro-3-(thiazol-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile

2-((2S)-4-(8-Fluoro-7-(2-fluoro-6-hydroxyphenyl)-2-(((S)-1-methylpyrrolidin-2-yl)methoxy)quinazolin-4-yl)piperazin-2-yl)acetonitrile (15 mg, 0.030 mmol) and (Z)-2-fluoro-3-(thiazol-2-yl)acrylic acid (5.3 mg, 0.030 mmol) were combined as solids and dissolved in DMF (1.0 mL). 1-Methylimidazole (24 μL, 0.30 mmol) was added followed by chloro-N,N,N′,N′-tetramethylformamidinium hexafluorophosphate (8.5 mg, 0.030 mmol). The reaction mixture was stirred at room temperature for 5 min. A saturated aqueous solution of ammonium hydroxide (100 L) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 90% 5:95 MeCN:H₂O with 0.1% TFA/10% 95:5 MeCN:H₂O with 0.1% TFA→100% 95:5 MeCN:H₂O with 0.1% TFA; λ=220 nm) to provide the desired product as the bis TFA salt (11 mg, 0.013 mmol, 43% yield). LC/MS (ESI) m/z: [M+H]⁺ calcd for C₃₂H₃₁F₃N₇₀₃S 650.2. found 650.3; ¹H NMR (500 MHz, DMSO-d₆) δ 10.28-10.14 (m, 1H), 9.86-9.71 (m, 1H), 8.03 (d, J=6.7 Hz, 1H), 7.92 (d, J=8.6 Hz, 1H), 7.37-7.28 (m, 2H), 7.18-7.05 (m, 1H), 6.83 (d, J=39 Hz, 1H), 5.05-4.87 (m, 1H), 4.86-4.70 (m, 1H), 4.68-4.58 (m, 1H), 4.41-4.27 (m, 2H), 4.18 (s, 1H), 3.97 (s, 1H), 3.93-3.81 (m, 2H), 3.69-3.46 (m, 4H), 2.99 (br d, J=3.8 Hz, 2H), 2.87-2.74 (m, 1H), 2.55-2.52 (m, 1H), 2.48 (br s, 1H), 2.33-2.21 (m, 1H), 2.12-2.02 (m, 1H), 2.01-1.87 (m, 2H).

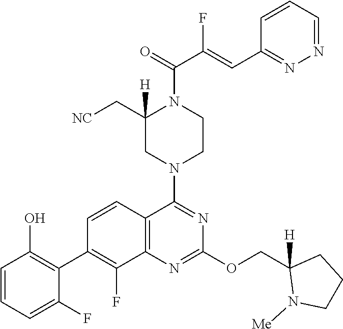 [SMILES: CN1CCC[C@H]1COc1nc(N2CCN(C(=O)/C(F)=C/c3cccnn3)[C@@H](CC#N)C2)c2ccc(-c3c(O)cccc3F)c(F)c2n1]

### Example 57

### 2-((2S)-1-((Z)-2-fluoro-3-(pyridazin-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile

2-((2S)-4-(8-Fluoro-7-(2-fluoro-6-hydroxyphenyl)-2-(((S)-1-methylpyrrolidin-2-yl)methoxy)quinazolin-4-yl)piperazin-2-yl)acetonitrile (15 mg, 0.030 mmol) and (Z)-2-fluoro-3-(pyridazin-2-yl)acrylic acid (5.1 mg, 0.030 mmol) were combined as solids and dissolved in DMF (1.0 mL). 1-Methylimidazole (24 μL, 0.30 mmol) was added followed by chloro-N,N,N′,N′-tetramethylformamidinium hexafluorophosphate (8.5 mg, 0.030 mmol). The reaction mixture was stirred at room temperature for 5 min. A saturated aqueous solution of ammonium hydroxide (100 L) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 95% 5:95 MeCN:H₂O with 0.1% TFA/5% 95:5 MeCN:H₂O with 0.1% TFA→100% 95:5 MeCN:H₂O with 0.1% TFA; λ=220 nm) to provide the desired product as the bis TFA salt (8 mg, 0.013 mmol, 30% yield). LC/MS (ESI) m/z: [M+H]⁺ calcd for C₃₃H₃₂F₃N₈O₃ 645.3. found 645.7; ¹H NMR (500 MHz, CD₃OD) δ 9.16 (dd, J=4.9, 1.5 Hz, 1H), 7.91 (d, J=8.6 Hz, 1H), 7.82 (dd, J=8.8, 5.1 Hz, 1H), 7.39 (t, J=7.2 Hz, 1H), 7.34-7.21 (m, 2H), 7.08-6.99 (m, 1H), 6.82-6.70 (m, 2H), 4.96 (s, 1H), 4.84-4.81 (m, 3H), 4.78 (br d, J=16.0 Hz, 3H), 4.71-4.50 (m, 3H), 4.15 (t, J=6.6 Hz, 1H), 3.50 (br s, 1H), 3.30-3.13 (m, 2H), 2.96-2.89 (m, 2H), 2.88-2.72 (m, 1H), 2.08 (s, 1H), 1.66 (s, 1H), 1.32 (br s, 2H), 1.01-0.82 (m, 1H).

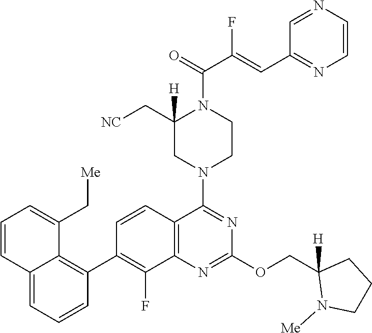 [SMILES: CCc1cccc2cccc(-c3ccc4c(N5CCN(C(=O)/C(F)=C/c6cnccn6)[C@@H](CC#N)C5)nc(OC[C@@H]5CCCN5C)nc4c3F)c12]

### Example 58

### 2-((S)-4-(7-(8-ethylnaphthalen-1-yl)-8-fluoro-2-(((S)-1-methylpyrrolidin-2-yl)methoxy)quinazolin-4-yl)-1-((Z)-2-fluoro-3-(pyrazin-2-yl)acryloyl)piperazin-2-yl)acetonitrile

2-((S)-4-(7-(8-Ethylnaphthalen-1-yl)-8-fluoro-2-(((S)-1-methylpyrrolidin-2-yl)methoxy)quinazolin-4-yl)piperazin-2-yl)acetonitrile (7 mg, 0.013 mmol) and (Z)-2-fluoro-3-(pyrazin-2-yl)acrylic acid (2.2 mg, 0.013 mmol) were combined as solids and dissolved in DMF (1.0 mL). 1-Methylimidazole (10 μL, 0.13 mmol) was added followed by chloro-N,N,N′,N′-tetramethylformamidinium hexafluorophosphate (3.7 mg, 0.013 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (5.4 mg, 0.008 mmol, 59% yield). LC/MS (ESI) m/z: [M+H]⁺ calcd for C₃₉H₃₉F₂N₈O₂ 689.3. found 689.4; ¹H NMR (500 MHz, DMSO-d₆) δ 8.99 (s, 1H), 8.75 (s, 1H), 8.63 (d, J=2.1 Hz, 1H), 8.07 (d, J=7.7 Hz, 1H), 8.01-7.88 (m, 2H), 7.65-7.48 (m, 1H), 7.41 (br dd, J=7.1, 2.3 Hz, 1H), 7.35 (br d, J=7.0 Hz, 1H), 7.24 (s, 1H), 7.13 (s, 1H), 7.03 (s, 1H), 4.83-4.69 (m, 1H), 4.61 (br dd, J=12.8, 6.9 Hz, 1H), 4.21 (s, 1H), 3.92-3.83 (m, 4H), 3.32-3.22 (m, 2H), 3.21-3.09 (m, 3H), 2.98 (s, 2H), 2.64-2.52 (m, 1H), 2.49-2.37 (m, 2H), 2.28 (br d, J=8.4 Hz, 1H), 2.12-2.03 (m, 2H), 2.01-1.84 (m, 2H), 1.28-1.11 (m, 1H), 0.84 (td, J=7.4, 2.9 Hz, 3H).

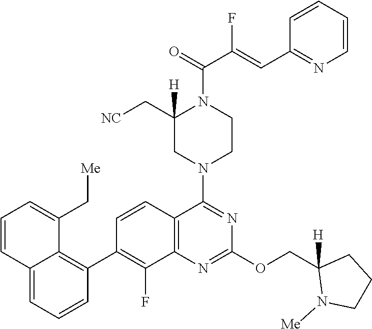 [SMILES: CCc1cccc2cccc(-c3ccc4c(N5CCN(C(=O)/C(F)=C/c6ccccn6)[C@@H](CC#N)C5)nc(OC[C@@H]5CCCN5C)nc4c3F)c12]

### Example 59

### 2-((S)-4-(7-(8-ethylnaphthalen-1-yl)-8-fluoro-2-(((S)-1-methylpyrrolidin-2-yl)methoxy)quinazolin-4-yl)-1-((Z)-2-fluoro-3-(pyridin-2-yl)acryloyl)piperazin-2-yl)acetonitrile

2-((S)-4-(7-(8-Ethylnaphthalen-1-yl)-8-fluoro-2-(((S)-1-methylpyrrolidin-2-yl)methoxy)quinazolin-4-yl)piperazin-2-yl)acetonitrile (7 mg, 0.013 mmol) and (Z)-2-fluoro-3-(pyridin-2-yl)acrylic acid (2.2 mg, 0.013 mmol) were combined as solids and dissolved in DMF (1.0 mL). 1-Methylimidazole (10 μL, 0.13 mmol) was added followed by chloro-N,N,N′,N′-tetramethylformamidinium hexafluorophosphate (3.7 mg, 0.013 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 82% 5:95 MeCN:H₂O with 0.1% TFA/18% 95:5 MeCN:H₂O with 0.1% TFA→100% 95:5 MeCN:H₂O with 0.1% TFA; λ=220 nm) to provide the desired product as the bis TFA salt (3.8 mg, 0.004 mmol, 32% yield). LC/MS (ESI) m/z: [M+H]⁺ calcd for C₄₀H₄₀F₂N₇O₂ 688.3. found 688.3; ¹H NMR (500 MHz, DMSO-d₆) δ 8.67 (br d, J=3.7 Hz, 1H), 8.07 (d, J=8.5 Hz, 1H), 8.00-7.88 (m, 2H), 7.81 (d, J=8.0 Hz, 1H), 7.59 (t, J=7.7 Hz, 1H), 7.53 (t, J=7.6 Hz, 2H), 7.44-7.32 (m, 2H), 7.28-7.18 (m, 1H), 7.14-7.07 (m, 1H), 7.04-6.98 (m, 1H), 4.74 (br s, 1H), 4.40 (br s, 1H), 3.91 (s, 3H), 3.75-3.55 (m, 2H), 3.55-3.50 (m, 1H), 3.30-3.22 (m, 3H), 3.21-3.10 (m, 3H), 2.98 (br s, 3H), 2.49-2.38 (m, 2H), 2.08 (s, 2H), 2.00-1.85 (m, 2H), 0.84 (td, J=7.3, 3.0 Hz, 3H).

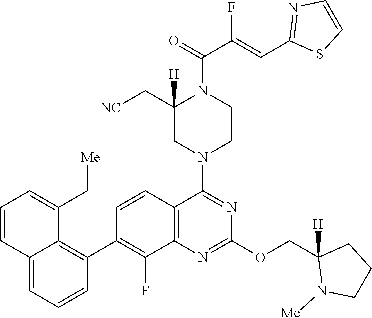 [SMILES: CCc1cccc2cccc(-c3ccc4c(N5CCN(C(=O)/C(F)=C/c6nccs6)[C@@H](CC#N)C5)nc(OC[C@@H]5CCCN5C)nc4c3F)c12]

### Example 60

### 2-((S)-4-(7-(8-ethylnaphthalen-1-yl)-8-fluoro-2-(((S)-1-methylpyrrolidin-2-yl)methoxy)quinazolin-4-yl)-1-((Z)-2-fluoro-3-(thiazol-2-yl)acryloyl)piperazin-2-yl)acetonitrile

2-((S)-4-(7-(8-Ethylnaphthalen-1-yl)-8-fluoro-2-(((S)-1-methylpyrrolidin-2-yl)methoxy)quinazolin-4-yl)piperazin-2-yl)acetonitrile (7 mg, 0.013 mmol) and (Z)-2-fluoro-3-(thiazol-2-yl)acrylic acid (2.3 mg, 0.013 mmol) were combined as solids and dissolved in DMF (1.0 mL). 1-Methylimidazole (10 μL, 0.13 mmol) was added followed by chloro-N,N,N′,N′-tetramethylformamidinium hexafluorophosphate (3.7 mg, 0.013 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product as the bis TFA salt (3.4 mg, 0.004 mmol, 28% yield). LC/MS (ESI) m/z: [M+H]⁺ calcd for C₃₈H₃₈F₂N₇O₂S 694.3. found 694.2; ¹H NMR (500 MHz, DMSO-d₆) δ 8.09-7.91 (m, 4H), 7.59 (t, J=7.6 Hz, 1H), 7.53 (t, J=7.8 Hz, 1H), 7.41 (br d, J=6.7 Hz, 1H), 7.35 (br d, J=6.9 Hz, 1H), 7.24 (s, 1H), 7.17-7.11 (m, 1H), 7.03 (s, 1H), 4.75 (br d, J=12.4 Hz, 1H), 4.61 (br dd, J=12.5, 6.4 Hz, 1H), 4.21 (s, 1H), 3.89-3.79 (m, 1H), 3.78-3.68 (m, 1H), 3.67-3.49 (m, 3H), 3.29-3.24 (m, 2H), 3.22-3.08 (m, 2H), 3.03-2.93 (m, 3H), 2.85 (br d, J=4.0 Hz, 2H), 2.56-2.52 (m, 1H), 2.49-2.36 (m, 2H), 2.27 (br dd, J=9.5, 2.8 Hz, 1H), 2.12-2.03 (m, 1H), 2.00-1.86 (m, 1H), 0.84 (td, J=7.4, 2.4 Hz, 3H).

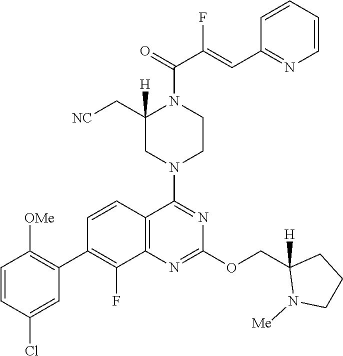 [SMILES: COc1ccc(Cl)cc1-c1ccc2c(N3CCN(C(=O)/C(F)=C/c4ccccn4)[C@@H](CC#N)C3)nc(OC[C@@H]3CCCN3C)nc2c1F]

### Example 61

### 2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(pyridin-2-yl)acryloyl)piperazin-2-yl)acetonitrile

2-((S)-4-(7-(5-Chloro-2-methoxyphenyl)-8-fluoro-2-(((S)-1-methylpyrrolidin-2-yl)methoxy)quinazolin-4-yl)piperazin-2-yl)acetonitrile (10 mg, 0.019 mmol) and (Z)-2-fluoro-3-(pyridin-2-yl)acrylic acid (3.2 mg, 0.019 mmol) were combined as solids and dissolved in DMF (1.0 mL). 1-Methylimidazole (15 μL, 0.19 mmol) was added followed by chloro-N,N,N′,N′-tetramethylformamidinium hexafluorophosphate (5.3 mg, 0.019 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (7.4 mg, 0.012 mmol, 57% yield). LC/MS (ESI) m/z: [M+H]⁺ calcd for C₃₅H₃₅ClF₂N₇O₃ 674.2; found 674.2; ¹H NMR (500 MHz, DMSO-d₆) δ 8.66 (br d, J=4.7 Hz, 1H), 7.96-7.85 (m, 2H), 7.80 (d, J=8.0 Hz, 1H), 7.52 (dd, J=8.9, 2.6 Hz, 1H), 7.43-7.36 (m, 2H), 7.28 (t, J=7.6 Hz, 1H), 7.23 (d, J=8.7 Hz, 1H), 6.65 (d, J=33 Hz, 1H), 4.40 (br dd, J=10.7, 4.6 Hz, 1H), 4.29 (br d, J=12.1 Hz, 2H), 4.21 (br dd, J=10.8, 6.3 Hz, 2H), 3.79 (s, 3H), 3.73-3.56 (m, 1H), 3.25-3.06 (m, 1H), 2.99-2.94 (m, 2H), 2.90 (s, 1H), 2.74 (s, 1H), 2.65-2.52 (m, 2H), 2.49-2.33 (m, 1H), 2.28-2.13 (m, 1H), 1.97-1.87 (m, 2H), 1.76 (s, 1H), 1.73-1.58 (m, 3H).

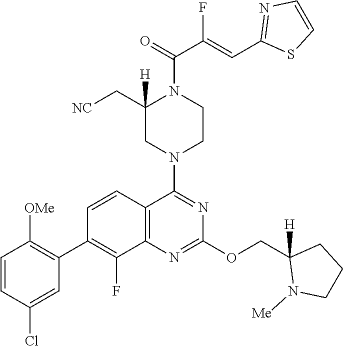 [SMILES: COc1ccc(Cl)cc1-c1ccc2c(N3CCN(C(=O)/C(F)=C/c4nccs4)[C@@H](CC#N)C3)nc(OC[C@@H]3CCCN3C)nc2c1F]

### Example 62

### 2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(thiazol-2-yl)acryloyl)piperazin-2-yl)acetonitrile

2-((S)-4-(7-(5-Chloro-2-methoxyphenyl)-8-fluoro-2-(((S)-1-methylpyrrolidin-2-yl)methoxy)quinazolin-4-yl)piperazin-2-yl)acetonitrile (10 mg, 0.019 mmol) and (Z)-2-fluoro-3-(thiazol-2-yl)acrylic acid (3.3 mg, 0.019 mmol) were combined as solids and dissolved in DMF (1.0 mL). 1-Methylimidazole (15 μL, 0.19 mmol) was added followed by chloro-N,N,N′,N′-tetramethylformamidinium hexafluorophosphate (5.3 mg, 0.019 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (6.9 mg, 0.011 mmol, 52% yield). LC/MS (ESI) m/z: [M+H]⁺ calcd for C₃₃H₃₃ClF₂N₇O₃S 680.2; found 680.1; ¹H NMR (500 MHz, DMSO-d₆) δ 8.06-7.94 (m, 2H), 7.86 (d, J=9.0 Hz, 1H), 7.55-7.48 (m, 1H), 7.38 (d, J=2.5 Hz, 1H), 7.27 (br t, J=7.5 Hz, 1H), 7.26-7.19 (m, 1H), 7.08 (d, J=37.5 Hz, 1H), 5.06-4.80 (m, 1H), 4.43-4.35 (m, 1H), 4.34-4.25 (m, 2H), 4.18 (br s, 2H), 3.77 (s, 3H), 3.61-3.57 (m, 3H), 3.11 (br d, J=5.6 Hz, 2H), 2.95 (br d, J=4.3 Hz, 1H), 2.61-2.55 (m, 1H), 2.37 (s, 3H), 2.18 (q, J=8.8 Hz, 1H), 1.98-1.92 (m, 1H), 1.73-1.58 (m, 3H).

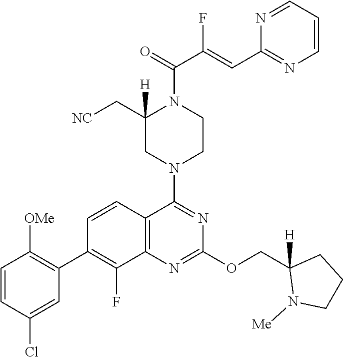 [SMILES: COc1ccc(Cl)cc1-c1ccc2c(N3CCN(C(=O)/C(F)=C/c4ncccn4)[C@@H](CC#N)C3)nc(OC[C@@H]3CCCN3C)nc2c1F]

### Example 63

### 2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(pyrimidin-2-yl)acryloyl)piperazin-2-yl)acetonitrile

2-((S)-4-(7-(5-Chloro-2-methoxyphenyl)-8-fluoro-2-(((S)-1-methylpyrrolidin-2-yl)methoxy)quinazolin-4-yl)piperazin-2-yl)acetonitrile (10 mg, 0.019 mmol) and (Z)-2-fluoro-3-(pyrimidin-2-yl)acrylic acid (3.2 mg, 0.019 mmol) were combined as solids and dissolved in DMF (1.0 mL). 1-Methylimidazole (15 μL, 0.19 mmol) was added followed by chloro-N,N,N′,N′-tetramethylformamidinium hexafluorophosphate (5.3 mg, 0.019 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product as the bis TFA salt (9.9 mg, 0.011 mmol, 58% yield). LC/MS (ESI) m/z: [M+H]⁺ calcd for C₃₄H₃₄ClF₂N₈O₃ 675.2. found 675.3; ¹H NMR (500 MHz, DMSO-d₆) δ 8.90 (d, J=4.7 Hz, 1H), 7.92 (m, 1H), 7.51 (m, 1H), 7.40-7.30 (m, 3H), 7.26-7.21 (m, 1H), 7.09 (d, J=36 Hz, 1H), 4.40 (br dd, J=10.7, 4.6 Hz, 1H), 4.39-4.29 (m, 4H), 3.77 (s, 3H), 3.25-3.06 (m, 1H), 2.99-2.94 (m, 2H), 2.90 (m, 2H), 2.74 (s, 1H), 2.65-2.52 (m, 2H), 2.49-2.33 (m, 1H), 2.28-2.13 (m, 1H), 1.97-1.87 (m, 2H), 1.76 (s, 2H), 1.73-1.58 (m, 3H).

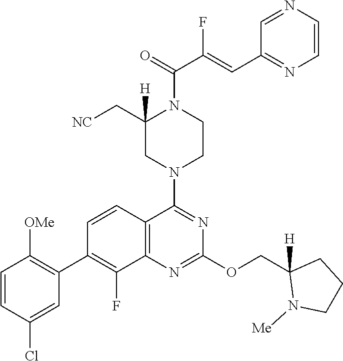 [SMILES: COc1ccc(Cl)cc1-c1ccc2c(N3CCN(C(=O)/C(F)=C/c4cnccn4)[C@@H](CC#N)C3)nc(OC[C@@H]3CCCN3C)nc2c1F]

### Example 64

### 2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(piperazin-2-yl)acryloyl)piperazin-2-yl)acetonitrile

2-((S)-4-(7-(5-Chloro-2-methoxyphenyl)-8-fluoro-2-(((S)-1-methylpyrrolidin-2-yl)methoxy)quinazolin-4-yl)piperazin-2-yl)acetonitrile (10 mg, 0.019 mmol) and (Z)-2-fluoro-3-(piperazin-2-yl)acrylic acid (3.2 mg, 0.019 mmol) were combined as solids and dissolved in DMF (1.0 mL). 1-Methylimidazole (15 μL, 0.19 mmol) was added followed by chloro-N,N,N′,N′-tetramethylformamidinium hexafluorophosphate (5.3 mg, 0.019 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product as the bis TFA salt (5.3 mg, 0.08 mmol, 41% yield). LC/MS (ESI) m/z: [M+H]⁺ calcd for C₃₄H₃₄ClF₂N₈O₃ 675.2. found 675.3; ¹H NMR (500 MHz, DMSO-d₆) δ 8.98 (s, 1H), 8.75 (s, 1H), 8.62 (d, J=2.4 Hz, 1H), 7.87 (br d, J=8.6 Hz, 1H), 7.52 (dd, J=8.9, 2.7 Hz, 1H), 7.39 (d, J=2.5 Hz, 1H), 7.29 (t, J=7.7 Hz, 1H), 7.22 (d, J=8.7 Hz, 1H), 6.73 (d, J=34 Hz, 1H), 4.40 (br dd, J=10.6, 4.7 Hz, 1H), 4.35-4.25 (m, 1H), 4.21 (br dd, J=10.8, 6.3 Hz, 1H), 3.77 (s, 3H), 2.99-2.92 (m, 2H), 2.90 (s, 1H), 2.74-2.52 (m, 2H), 2.49-2.30 (m, 3H), 2.24-2.15 (m, 2H), 1.98-1.84 (m, 4H), 1.76 (s, 1H), 1.73-1.59 (m, 3H).

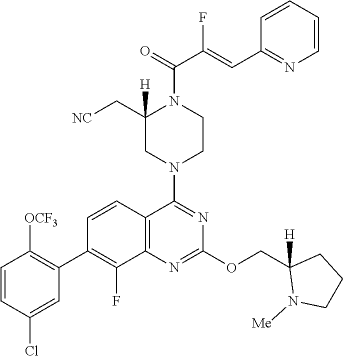 [SMILES: CN1CCC[C@H]1COc1nc(N2CCN(C(=O)/C(F)=C/c3ccccn3)[C@@H](CC#N)C2)c2ccc(-c3cc(Cl)ccc3OC(F)(F)F)c(F)c2n1]

### Example 65

### 2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(pyridin-2-yl)acryloyl)piperazin-2-yl)acetonitrile

2-((S)-4-(7-(5-Chloro-2-(trifluoromethoxy)phenyl)-8-fluoro-2-(((S)-1-methylpyrrolidin-2-yl)methoxy)quinazolin-4-yl)piperazin-2-yl)acetonitrile (10 mg, 0.017 mmol) and (Z)-2-fluoro-3-(pyridin-2-yl)acrylic acid (2.9 mg, 0.017 mmol) were combined as solids and dissolved in DMF (1.0 mL). 1-Methylimidazole (14 μL, 0.17 mmol) was added followed by chloro-N,N,N′,N′-tetramethylformamidinium hexafluorophosphate (4.9 mg, 0.017 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 71% 5:95 MeCN:H₂O with 10 mM AA/29% 95:5 MeCN:H₂O with 10 mM AA→100% 95:5 MeCN:H₂O with 10 mM AA; λ=220 nm) to provide the desired product (5.8 mg, 0.008 mmol, 45% yield). LC/MS (ESI) m/z: [M+H]⁺ calcd for C₃₅H₃₂ClF₅N₇O₃ 728.2. found 728.2; ¹H NMR (500 MHz, DMSO-d₆) δ 8.66 (br d, J=4.6 Hz, 1H), 7.95 (d, J=8.3 Hz, 1H), 7.91 (t, J=7.7 Hz, 1H), 7.80 (d, J=7.9 Hz, 1H), 7.76-7.70 (m, 2H), 7.63 (br d, J=8.9 Hz, 1H), 7.39 (t, J=6.4 Hz, 1H), 7.34 (t, J=7.3 Hz, 1H), 6.64 (d, J=37 Hz, 1H), 4.40 (dd, J=10.8, 5.0 Hz, 2H), 4.31 (br d, J=13.3 Hz, 1H), 4.22 (br dd, J=10.6, 6.2 Hz, 2H), 3.91 (s, 2H), 3.12-3.17 (m, 2H), 3.20-3.02 (m, 2H), 2.99-2.94 (m, 2H), 2.37 (s, 3H), 2.28-2.07 (m, 1H), 1.73-1.60 (m, 3H), 1.04 (d, J=6.0 Hz, 1H).

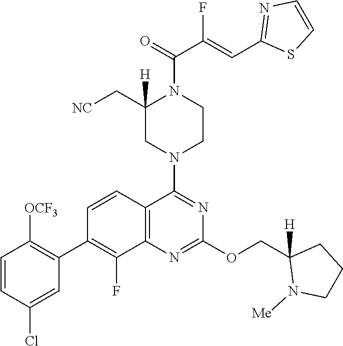 [SMILES: CN1CCC[C@H]1COc1nc(N2CCN(C(=O)/C(F)=C/c3nccs3)[C@@H](CC#N)C2)c2ccc(-c3cc(Cl)ccc3OC(F)(F)F)c(F)c2n1]

### Example 66

### 2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(thiazol-2-yl)acryloyl)piperazin-2-yl)acetonitrile

2-((S)-4-(7-(5-Chloro-2-(trifluoromethoxy)phenyl)-8-fluoro-2-(((S)-1-methylpyrrolidin-2-yl)methoxy)quinazolin-4-yl)piperazin-2-yl)acetonitrile (10 mg, 0.017 mmol) and (Z)-2-fluoro-3-(thiazol-2-yl)acrylic acid (3.0 mg, 0.017 mmol) were combined as solids and dissolved in DMF (1.0 mL). 1-Methylimidazole (14 μL, 0.17 mmol) was added followed by chloro-N,N,N′,N′-tetramethylformamidinium hexafluorophosphate (4.9 mg, 0.017 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 75% 5:95 MeCN:H₂O with 0.1% TFA/25% 95:5 MeCN:H₂O with 0.1% TFA→100% 95:5 MeCN:H₂O with 0.1% TFA; λ=220 nm) to provide the desired product as the bis TFA salt (11.6 mg, 0.012 mmol, 71% yield). LC/MS (ESI) m/z: [M+H]⁺ calcd for C₃₃H₃₀ClF₅N₇O₃S 734.2. found 734.1; ¹H NMR (500 MHz, DMSO-d₆) δ 8.05-7.95 (m, 2H), 7.77-7.70 (m, 1H), 7.65-7.63 (m, 1H), 7.42-7.39 (m, 1H), 7.24 (s, br, 1H), 7.13 (s, 1H), 7.05 (d, J=35 Hz, 1H), 4.77-4.74 (m, 1H), 4.65-4.60 (m, 1H), 4.30 (m, 1H), 4.01-3.80 (m, 1H), 3.72-3.53 (m, 4H), 3.23-3.07 (m, 2H), 2.98 (br s, 2H), 2.57-2.52 (m, 1H), 2.31-2.25 (m, 1H), 2.12-2.02 (m, 1H), 1.99-1.87 (m, 3H), 1.04 (d, J=6.1 Hz, 1H), 1.05-1.2 (m, 1H).

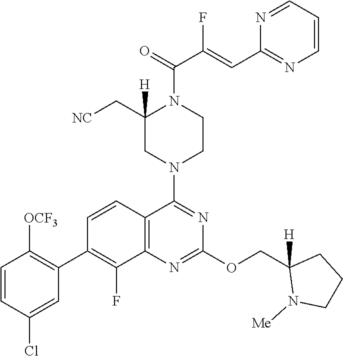 [SMILES: CN1CCC[C@H]1COc1nc(N2CCN(C(=O)/C(F)=C/c3ncccn3)[C@@H](CC#N)C2)c2ccc(-c3cc(Cl)ccc3OC(F)(F)F)c(F)c2n1]

### Example 67

### 2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(pyrimidin-2-yl)acryloyl)piperazin-2-yl)acetonitrile

2-((S)-4-(7-(5-Chloro-2-(trifluoromethoxy)phenyl)-8-fluoro-2-(((S)-1-methylpyrrolidin-2-yl)methoxy)quinazolin-4-yl)piperazin-2-yl)acetonitrile (10 mg, 0.017 mmol) and (Z)-2-fluoro-3-(pyrimidin-2-yl)acrylic acid (2.9 mg, 0.017 mmol) were combined as solids and dissolved in DMF (1.0 mL). 1-Methylimidazole (14 μL, 0.17 mmol) was added followed by chloro-N,N,N′,N′-tetramethylformamidinium hexafluorophosphate (4.9 mg, 0.017 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 0.1% TFA/21% 95:5 MeCN:H₂O with 0.1% TFA→100% 95:5 MeCN:H₂O with 0.1% TFA; λ=220 nm) to provide the desired product as the bis TFA salt (8.4 mg, 0.009 mmol, 66% yield). LC/MS (ESI) m/z: [M+H]⁺ calcd for C₃₄H₃₁ClF₅N₈O₃ 729.2. found 729.2; ¹H NMR (500 MHz, DMSO-d₆) δ 8.90 (d, J=4.9 Hz, 2H), 8.00 (d, J=8.6 Hz, 1H), 7.76-7.71 (m, 1H), 7.64 (br d, J=8.4 Hz, 1H), 7.49-7.45 (m, 1H), 7.40 (t, J=7.5 Hz, 1H), 7.31-7.19 (m, 1H), 7.14-7.04 (br s, 1H), 4.85-4.69 (m, 1H), 4.61 (br dd, J=12.6, 7.1 Hz, 1H), 4.41-4.27 (m, 2H), 4.19 (s, 1H), 4.15-4.04 (m, 1H), 3.97 (s, 1H), 3.91 (s, 2H), 3.83 (br d, J=1.6 Hz, 1H), 3.73-3.51 (m, 2H), 3.50-3.39 (m, 2H), 2.85 (s, 1H), 2.56-2.52 (m, 1H), 2.32-2.22 (m, 1H), 2.11-2.03 (m, 2H), 2.01-1.86 (m, 2H).

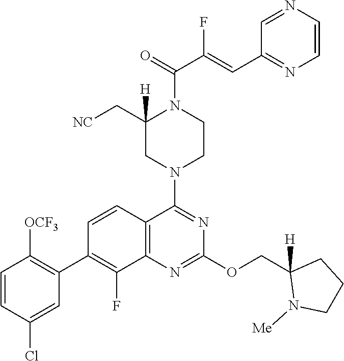 [SMILES: CN1CCC[C@H]1COc1nc(N2CCN(C(=O)/C(F)=C/c3cnccn3)[C@@H](CC#N)C2)c2ccc(-c3cc(Cl)ccc3OC(F)(F)F)c(F)c2n1]

### Example 68

### 2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(pyrazin-2-yl)acryloyl)piperazin-2-yl)acetonitrile

2-((S)-4-(7-(5-Chloro-2-(trifluoromethoxy)phenyl)-8-fluoro-2-(((S)-1-methylpyrrolidin-2-yl)methoxy)quinazolin-4-yl)piperazin-2-yl)acetonitrile (10 mg, 0.017 mmol) and (Z)-2-fluoro-3-(pyrazin-2-yl)acrylic acid (2.9 mg, 0.017 mmol) were combined as solids and dissolved in DMF (1.0 mL). 1-Methylimidazole (14 μL, 0.17 mmol) was added followed by chloro-N,N,N′,N′-tetramethylformamidinium hexafluorophosphate (4.9 mg, 0.017 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 71% 5:95 MeCN:H₂O with 10 mM AA/29% 95:5 MeCN:H₂O with 10 mM AA→100% 95:5 MeCN:H₂O with 10 mM AA; λ=220 nm) to provide the desired product (7.9 mg, 0.009 mmol, 62% yield). LC/MS (ESI) m/z: [M+H]⁺ calcd for C₃₄H₃₁ClF₅N₈O₃ 729.2. found 729.1; ¹H NMR (500 MHz, DMSO-d₆) δ 8.98 (s, 1H), 8.75 (s, 1H), 8.62 (d, J=2.3 Hz, 1H), 7.95 (br d, J=8.6 Hz, 1H), 7.76-7.71 (m, 2H), 7.63 (br d, J=9.3 Hz, 1H), 7.34 (br s, 1H), 6.73 (d, J=37 Hz, 1H), 4.39 (br d, J=4.7 Hz, 1H), 4.31 (br d, J=13.4 Hz, 2H), 4.23 (dd, J=10.6, 6.3 Hz, 2H), 3.80-3.60 (m, 1H), 3.20-3.04 (m, 2H), 3.03-2.82 (m, 3H), 2.59 (br s, 2H), 2.57-2.52 (m, 1H), 2.49-2.33 (m, 2H), 2.19 (br d, J=7.9 Hz, 1H), 1.76 (s, 1H), 1.73-1.60 (m, 2H), 1.04 (d, J=6.0 Hz, 1H).

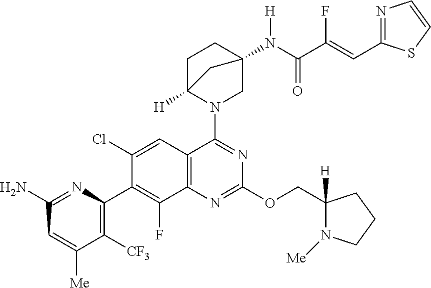

### Example 69

### (Z)-N-((1S,4S)-2-(7M-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-2-azabicyclo[2.2.1]heptan-4-yl)-2-fluoro-3-(thiazol-2-yl)acrylamide

(1S,4S)-2-(7M-(6-Amino-4-methyl-3-(trifluoromethyl)pyridin-2-yl)-6-chloro-8-fluoro-2-(((S)-1-methylpyrrolidin-2-yl)methoxy)quinazolin-4-yl)-2-azabicyclo[2.2.1]heptan-4-amine (40 mg, 0.069 mmol) and (Z)-2-fluoro-3-(thiazol-2-yl)acrylic acid (24 mg, 0.14 mmol) were combined as solids and dissolved in DMF (0.7 mL). 1-Methylimidazole (28 μL, 0.35 mmol) was added followed by chloro-N,N,N′,N′-tetramethylformamidinium hexafluorophosphate (39 mg, 0.14 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (5.6 mg, 0.007 mmol, 11% yield over two steps). LC/MS (ESI) m/z: [M+H]⁺ calcd for C₃₃H₃₃ClF₅N₈O₂S 735.2. found 735.2; ¹H NMR (500 MHz, DMSO-d₆) δ 9.31 (s, 1H), 8.06-7.99 (m, 2H), 7.93 (br s, 1H), 7.29 (d, J=36.2 Hz, 1H), 6.88-6.79 (m, 2H), 6.49 (s, 1H), 4.90 (br s, 1H), 4.46-4.23 (m, 2H), 4.16-4.03 (m, 2H), 3.00-2.89 (m, 2H), 2.62-2.55 (m, 1H), 2.37 (br s, 3H), 2.36 (s, 3H), 2.22-2.14 (m, 2H), 2.14-2.08 (m, 1H), 2.03-1.92 (m, 4H), 1.71-1.58 (m, 3H).

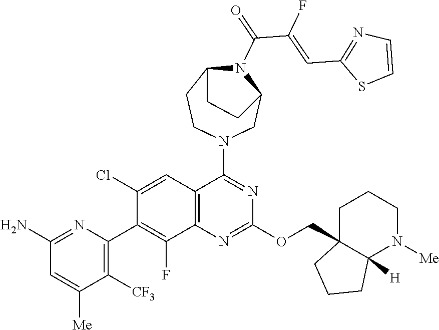 [SMILES: Cc1cc(N)nc(-c2c(Cl)cc3c(N4CC[C@@H]5CC[C@H](C4)N5C(=O)/C(F)=C/c4nccs4)nc(OC[C@]45CCC[C@H]4N(C)CCC5)nc3c2F)c1C(F)(F)F]

### Example 70

### (Z)-1-((1R,6S)-3-(7-(6-amino-4-methyl-3-(trifluoromethyl)pyridin-2-yl)-6-chloro-8-fluoro-2-(((4aS,7aR)-1-methyloctahydro-4aH-cyclopenta[b]pyridin-4a-yl)methoxy)quinazolin-4-yl)-3,9-diazabicyclo[4.2.1]nonan-9-yl)-2-fluoro-3-(thiazol-2-yl)prop-2-en-1-one

6-(2-(((4aS,7aR)-1-methyloctahydro-4aH-cyclopenta[b]pyridin-4a-yl)methoxy)-6-chloro-4-((1R,6S)-3,9-Diazabicyclo[4.2.1]nonan-3-yl)-8-fluoroquinazolin-7-yl)-4-methyl-5-(trifluoromethyl)pyridin-2-amine (prepared according to the procedure described in WO2022/192790, 3 mg, 0.005 mmol) and (Z)-2-fluoro-3-(thiazol-2-yl)acrylic acid (2.4 mg, 0.014 mmol) were combined as solids and dissolved in DMF (0.3 mL). 1-Methylimidazole (2 μL, 0.028 mmol) was added followed by chloro-N,N,N′,N′-tetramethylformamidinium hexafluorophosphate (3.9 mg, 0.014 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 70% 5:95 MeCN:H₂O with 10 mM AA/30% 95:5 MeCN:H₂O with 10 mM AA→100% 95:5 MeCN:H₂O with 10 mM AA; λ=220 nm) to provide the desired product (3 mg, 0.004 mmol, 81% yield). LC/MS (ESI) m/z: [M+H]⁺ calcd for C₃₈H₄₁ClF₅N₈O₂S 803.3. found 803.3; ¹H NMR (500 MHz, DMSO-d₆) δ 8.03-7.96 (m, 2H), 7.94-7.89 (m, 1H), 7.15 (d, J=37.8 Hz, 0.4H), 7.02 (d, J=36.3 Hz, 0.6H), 6.84 (s, 2H), 6.50 (s, 1H), 4.90-4.73 (m, 2H), 4.66-4.27 (m, 2H), 4.12-3.86 (m, 3H), 3.75-3.62 (m, 2H), 2.47-2.40 (m, 1H), 2.37 (br s, 3H), 2.31-1.99 (m, 7H), 1.88-1.76 (m, 2H), 1.72-1.32 (m, 11H).

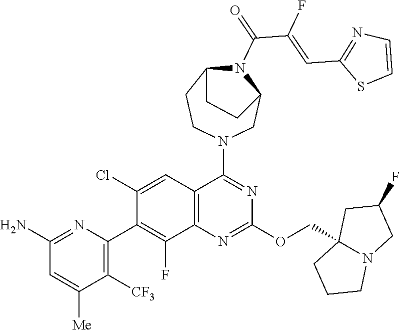 [SMILES: Cc1cc(N)nc(-c2c(Cl)cc3c(N4CC[C@@H]5CC[C@H](C4)N5C(=O)/C(F)=C/c4nccs4)nc(OC[C@@]45CCCN4C[C@H](F)C5)nc3c2F)c1C(F)(F)F]

### Example 71

### (Z)-1-((1R,6S)-3-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,9-diazabicyclo[4.2.1]nonan-9-yl)-2-fluoro-3-(thiazol-2-yl)prop-2-en-1-one

6-(2-(((2R,7aS)-2-fluorotetrahydro-1H-pyrrolizin-7a(5H)-yl)methoxy)-6-chloro-4-((1R,6S)-3,9-Diazabicyclo[4.2.1]nonan-3-yl)-8-fluoro-quinazolin-7-yl)-4-methyl-5-(trifluoromethyl)pyridin-2-amine (prepared according to the procedure described in WO2022/192790, 3 mg, 0.005 mmol) and (Z)-2-fluoro-3-(thiazol-2-yl)acrylic acid (2.4 mg, 0.014 mmol) were combined as solids and dissolved in DMF (0.3 mL). 1-Methylimidazole (2 μL, 0.028 mmol) was added followed by chloro-N,N,N′,N′-tetramethylformamidinium hexafluorophosphate (3.9 mg, 0.014 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 69% 5:95 MeCN:H₂O with 10 mM AA/31% 95:5 MeCN:H₂O with 10 mM AA→100% 95:5 MeCN:H₂O with 10 mM AA; λ=220 nm) to provide the desired product (2.4 mg, 0.003 mmol, 63% yield). LC/MS (ESI) m/z: [M+H]⁺ calcd for C₃₆H₃₆ClF₆N₈O₂S 793.2. found 793.0; ¹H NMR (500 MHz, DMSO-d₆) δ 8.03-7.98 (m, 2H), 7.96-7.89 (m, 1H), 7.16 (d, J=37.2 Hz, 0.4H), 7.01 (d, J=37.2 Hz, 0.6H), 6.84 (s, 2H), 6.50 (s, 1H), 5.34 (d, J=47.0 Hz, 1H), 4.90-4.60 (m, 3H), 4.42-3.93 (m, 4H), 3.79-3.47 (m, 4H), 3.06-2.84 (m, 1H), 2.47-2.40 (m, 1H), 2.37 (br s, 3H), 2.29-2.00 (m, 7H), 1.89-1.80 (m, 2H), 1.74-1.51 (m, 2H).

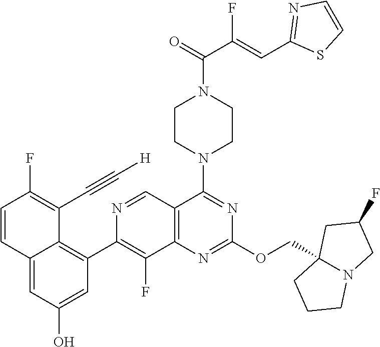 [SMILES: C#Cc1c(F)ccc2cc(O)cc(-c3ncc4c(N5CCN(C(=O)/C(F)=C/c6nccs6)CC5)nc(OC[C@@]56CCCN5C[C@H](F)C6)nc4c3F)c12]

### Example 72

### (Z)-1-(4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)piperazin-1-yl)-2-fluoro-3-(thiazol-2-yl)prop-2-en-1-one

5-Ethynyl-6-fluoro-4-(8-fluoro-2-(((2R,7aS)-2-fluorotetrahydro-1H-pyrrolizin-7a(5H)-yl)methoxy)-4-(piperazin-1-yl)quinazolin-7-yl)naphthalen-2-ol (5 mg, 0.009 mmol) and (Z)-2-fluoro-3-(thiazol-2-yl)acrylic acid (6.0 mg, 0.035 mmol) were combined as solids and dissolved in DMF (0.3 mL). 1-Methylimidazole (6 μL, 0.07 mmol) was added followed by chloro-N,N,N′,N′-tetramethylformamidinium hexafluorophosphate (9.8 mg, 0.035 mmol). The reaction mixture was stirred at room temperature for 5 min. A saturated aqueous solution of ammonium hydroxide (100 μL) was added, and the reaction mixture was stirred for an additional 5 min. The reaction mixture was directly purified by reverse phase HPLC (column: Xbridge C18, 19 mm×200 mm, 5 μm particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (2 mg, 0.003 mmol, 32% yield). LC/MS (ESI) m/z: [M+H]⁺ calcd for C₃₈H₃₃F₄N₆O₃S 729.2. found 729.3; ¹H NMR (500 MHz, DMSO-d₆) δ 10.15 (br s, 1H), 8.04-7.98 (m, 2H), 7.96 (dd, J=9.2, 6.0 Hz, 1H), 7.81 (d, J=8.5 Hz, 1H), 7.46 (t, J=9.0 Hz, 1H), 7.36 (d, J=2.4 Hz, 1H), 7.29-7.23 (m, 1H), 7.09 (d, J=37.5 Hz, 1H), 7.08 (s, 1H), 5.32 (d, J=54.4 Hz, 1H), 4.20-4.01 (m, 2H), 4.00-3.87 (m, 8H), 3.86 (s, 1H), 3.18-3.02 (m, 3H), 2.92-2.81 (m, 1H), 2.22-2.15 (m, 1H), 2.13-2.08 (m, 1H), 2.01 (br s, 1H), 1.89-1.74 (m, 3H).

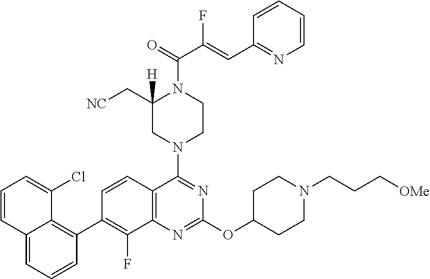 [SMILES: COCCCN1CCC(Oc2nc(N3CCN(C(=O)/C(F)=C/c4ccccn4)[C@@H](CC#N)C3)c3ccc(-c4cccc5cccc(Cl)c45)c(F)c3n2)CC1]

### Example 73

### (S,Z)-2-(4-(7-(8-chloronaphthalen-1-yl)-8-fluoro-2-((1-(3-methoxypropyl)piperidin-4-yl)oxy)quinazolin-4-yl)-1-(2-fluoro-3-(pyridin-2-yl)acryloyl)piperazin-2-yl)acetonitrile

(S)-2-(4-(7-(8-Chloronaphthalen-1-yl)-8-fluoro-2-((1-(3-methoxypropyl)piperidin-4-yl)oxy)quinazolin-4-yl)piperazin-2-yl)acetonitrile (10 mg, 0.017 mmol), (Z)-2-fluoro-3-(pyridin-2-yl)acrylic acid (2.8 mg, 0.017 mmol) were combined as solids and dissolved in DMF (1.0 mL). 1-Methylimidazole (13 μL, 0.17 mmol) was added followed by chloro-N,N,N′,N′-tetramethylformamidinium hexafluorophosphate (4.7 mg, 0.017 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (2 mg, 0.003 mmol, 16% yield). LC/MS (ESI) m/z: [M+H]⁺ calcd for C₄₁H₄₁ClF₂N₇O₃ 752.3. found 752.3; ¹H NMR (500 MHz, DMSO-d₆) δ 8.66 (br d, J=3.5 Hz, 1H), 8.13-8.05 (m, 1H), 8.01-7.87 (m, 2H), 7.85-7.79 (m, J=8.0 Hz, 1H), 7.75-7.63 (m, 2H), 7.61-7.50 (m, 3H), 7.44-7.31 (m, 2H), 6.65 (d, J=38 Hz, 1H), 5.13-4.90 (m, 2H), 4.38-4.23 (m, 2H), 3.99-3.85 (m, 3H), 3.70-3.55 (m, 3H), 3.29-3.10 (m, 4H), 2.87-2.67 (m, 2H), 2.49-2.33 (m, 2H), 2.33-2.18 (m, 2H), 2.18-1.99 (m, 3H), 1.82-1.62 (m, 3H), 1.24 (br s, 1H).

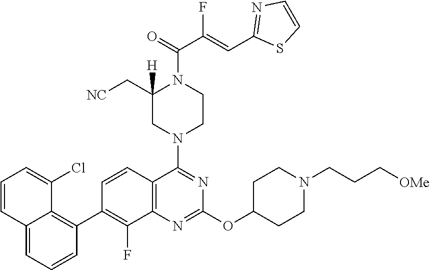 [SMILES: COCCCN1CCC(Oc2nc(N3CCN(C(=O)/C(F)=C/c4nccs4)[C@@H](CC#N)C3)c3ccc(-c4cccc5cccc(Cl)c45)c(F)c3n2)CC1]

### Example 74

### (S,Z)-2-(4-(7-(8-chloronaphthalen-1-yl)-8-fluoro-2-((1-(3-methoxypropyl)piperidin-4-yl)oxy)quinazolin-4-yl)-1-(2-fluoro-3-(thiazol-2-yl)acryloyl)piperazin-2-yl)acetonitrile

(S)-2-(4-(7-(8-Chloronaphthalen-1-yl)-8-fluoro-2-((1-(3-methoxypropyl)piperidin-4-yl)oxy)quinazolin-4-yl)piperazin-2-yl)acetonitrile (10 mg, 0.017 mmol), (Z)-2-fluoro-3-(thiazol-2-yl)acrylic acid (2.8 mg, 0.017 mmol) were combined as solids and dissolved in DMF (1.0 mL). 1-Methylimidazole (13 μL, 0.17 mmol) was added followed by chloro-N,N,N′,N′-tetramethylformamidinium hexafluorophosphate (4.7 mg, 0.017 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 81% 5:95 MeCN:H₂O with 0.1% TFA/19% 95:5 MeCN:H₂O with 0.1% TFA→100% 95:5 MeCN:H₂O with 0.1% TFA; λ=220 nm) to provide the desired product as the bis TFA salt (3.8 mg, 0.004 mmol, 23% yield). LC/MS (ESI) m/z: [M+H]⁺ calcd for C₃₉H₃₉ClF₂N₇O₃S 758.2. found 758.1; ¹H NMR (500 MHz, DMSO-d₆) δ 8.19-8.18 (m, 1H), 8.12-7.99 (m, 1H), 8.04-7.97 (m, 2H), 7.96-7.90 (m, 1H), 7.73-7.64 (m, 2H), 7.62-7.49 (m, 2H), 7.39-7.34 (m, 1H), 7.11 (d, J=37 Hz, 1H), 5.54-5.40 (m, 1H), 5.27-5.16 (m, 1H), 5.01-4.88 (m, 1H), 4.42-4.06 (m, 1H), 3.72-3.52 (m, 3H), 3.46-3.38 (m, 2H), 3.37-3.05 (m, 8H), 2.70 (s, 3H), 2.43-2.34 (m, 1H), 2.28-2.20 (m, 1H), 2.15-2.06 (m, 1H), 1.96-1.83 (m, 3H), 1.06-1.03 (m, 1H).

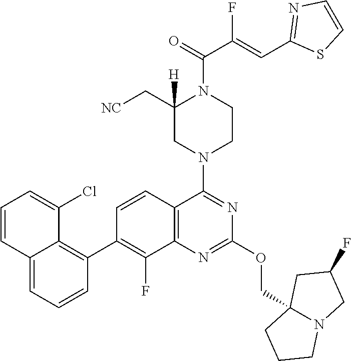 [SMILES: N#CC[C@H]1CN(c2nc(OC[C@@]34CCCN3C[C@H](F)C4)nc3c(F)c(-c4cccc5cccc(Cl)c45)ccc23)CCN1C(=O)/C(F)=C/c1nccs1]

### Example 75

### 2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(thiazol-2-yl)acryloyl)piperazin-2-yl)acetonitrile

2-((S)-4-(7-(8-Chloronaphthalen-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile, HCl (10 mg, 0.016 mmol) and (Z)-2-fluoro-3-(thiazol-2-yl)acrylic acid (2.8 mg, 0.016 mmol) were combined as solids and dissolved in DMF (1.0 mL). 1-Methylimidazole (13 μL, 0.16 mmol) was added followed by chloro-N,N,N′,N′-tetramethylformamidinium hexafluorophosphate (4.5 mg, 0.017 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 65% 5:95 MeCN:H₂O with 10 mM AA/35% 95:5 MeCN:H₂O with 10 mM AA→100% 95:5 MeCN:H₂O with 10 mM AA; λ=220 nm) to provide the desired product (3.7 mg, 0.005 mmol, 31% yield). LC/MS (ESI) m/z: [M+H]⁺ calcd for C₃₈H₃₄ClF₃N₇O₂S 744.2. found 744.0; ¹H NMR (500 MHz, DMSO-d₆) δ 8.17 (d, J=8.2 Hz, 1H), 8.09 (d, J=8.3 Hz, 1H), 8.05-7.99 (m, 2H), 7.94-7.88 (m, 1H), 7.70 (td, J=7.7, 2.9 Hz, 1H), 7.67-7.63 (m, 1H), 7.56 (td, J=7.8, 1.8 Hz, 1H), 7.52 (d, J=7.2 Hz, 1H), 7.37-7.29 (m, 1H), 7.09 (d, J=37.8 Hz, 1H), 5.27 (d, J=54.6 Hz, 1H), 5.08-4.84 (m, 1H), 4.40-4.26 (m, 2H), 4.19-3.98 (m, 4H), 3.70-3.57 (m, 1H), 3.15-3.05 (m, 4H), 3.04-2.97 (m, 2H), 2.86-2.78 (m, 1H), 2.19-2.10 (m, 1H), 2.09-2.05 (m, 1H), 2.04-1.99 (m, 1H), 1.87-1.74 (m, 3H).

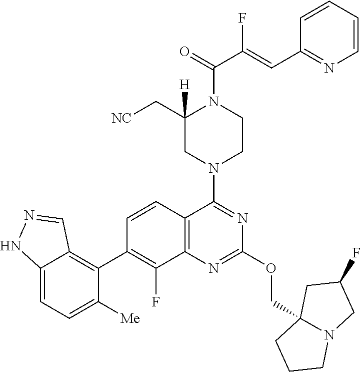 [SMILES: Cc1ccc2[nH]ncc2c1-c1ccc2c(N3CCN(C(=O)/C(F)=C/c4ccccn4)[C@@H](CC#N)C3)nc(OC[C@@]34CCCN3C[C@H](F)C4)nc2c1F]

### Example 76

### 2-((2S)-4-(8-fluoro-2-(((2R,7aS)-2-fluorotetrahydro-1H-pyrrolizin-7a(5H)-yl)methoxy)-7-(5-methyl-1H-indazol-4-yl)quinazolin-4-yl)-1-((Z)-2-fluoro-3-(pyridin-2-yl)acryloyl)piperazin-2-yl)acetonitrile

2-((2S)-4-(8-Fluoro-2-(((2R,7aS)-2-fluorotetrahydro-1H-pyrrolizin-7a(5H)-yl)methoxy)-7-(5-methyl-1H-indazol-4-yl)quinazolin-4-yl)piperazin-2-yl)acetonitrile, HCl (10 mg, 0.017 mmol) and (Z)-2-fluoro-3-(pyridin-2-yl)acrylic acid (2.8 mg, 0.017 mmol) were combined as solids and dissolved in DMF (1.0 mL). 1-Methylimidazole (13 μL, 0.17 mmol) was added followed by chloro-N,N,N′,N′-tetramethylformamidinium hexafluorophosphate (4.7 mg, 0.017 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (6.5 mg, 0.005 mmol, 31% yield). LC/MS (ESI) m/z: [M+H]⁺ calcd for C₃₈H₃₇F₃N₉O₂ 708.3. found 708.2; ¹H NMR (500 MHz, DMSO-d₆) δ 13.17 (br s, 1H), 8.66 (br d, J=4.4 Hz, 1H), 7.99 (br t, J=7.6 Hz, 1H), 7.91 (t, J=7.4 Hz, 1H), 7.81 (d, J=7.9 Hz, 1H), 7.61-7.53 (m, 2H), 7.42-7.30 (m, 3H), 6.69 (d, J=38 Hz, 1H), 5.35-5.24 (br m, 1H), 5.06-4.84 (m, 1H), 4.42-4.25 (m, 2H), 4.23-4.07 (m, 3H), 4.05 (br s, 1H), 3.64-3.57 (m, 1H), 3.55-3.37 (m, 1H), 3.28-3.23 (m, 1H), 3.22-3.04 (m, 4H), 2.96-2.80 (m, 1H), 2.25 (s, 3H), 2.21-2.00 (m, 3H), 1.92-1.76 (m, 3H).

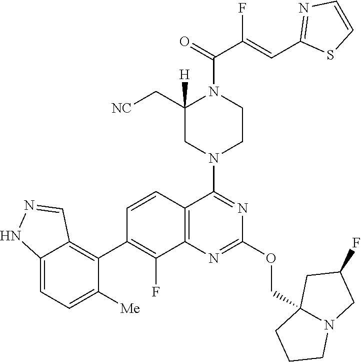 [SMILES: Cc1ccc2[nH]ncc2c1-c1ccc2c(N3CCN(C(=O)/C(F)=C/c4nccs4)[C@@H](CC#N)C3)nc(OC[C@@]34CCCN3C[C@H](F)C4)nc2c1F]

### Example 77

### 2-((2S)-4-(8-fluoro-2-(((2R,7aS)-2-fluorotetrahydro-1H-pyrrolizin-7a(5H)-yl)methoxy)-7-(5-methyl-1H-indazol-4-yl)quinazolin-4-yl)-1-((Z)-2-fluoro-3-(thiazol-2-yl)acryloyl)piperazin-2-yl)acetonitrile

2-((2S)-4-(8-Fluoro-2-(((2R,7aS)-2-fluorotetrahydro-1H-pyrrolizin-7a(5H)-yl)methoxy)-7-(5-methyl-1H-indazol-4-yl)quinazolin-4-yl)piperazin-2-yl)acetonitrile, HCl (10 mg, 0.017 mmol) and (Z)-2-fluoro-3-(thiazol-2-yl)acrylic acid (2.8 mg, 0.017 mmol) were combined as solids and dissolved in DMF (1.0 mL). 1-Methylimidazole (13 μL, 0.17 mmol) was added followed by chloro-N,N,N′,N′-tetramethylformamidinium hexafluorophosphate (4.7 mg, 0.017 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (6.5 mg, 0.005 mmol, 31% yield). LC/MS (ESI) m/z: [M+H]⁺ calcd for C₃₈H₃₇F₃N₉O₂ 708.3. found 708.2; ¹H NMR (500 MHz, DMSO-d₆) δ 13.17 (br s, 1H), 8.05-7.96 (m, 3H), 7.60-7.55 (m, 2H), 7.40-7.30 (m, 2H), 7.11 (d, J=36 Hz, 1H), 5.36-5.25 (br m, 1H), 4.36 (br d, J=13.7 Hz, 2H), 4.26-4.13 (m, 2H), 4.10 (br s, 2H), 4.00-3.82 (m, 1H), 3.65-3.43 (m, 3H), 3.19-3.05 (m, 1H), 2.86 (br dd, J=4.3, 1.9 Hz, 1H), 2.25 (s, 3H), 2.15 (br s, 1H), 2.12-2.00 (m, 3H), 1.93-1.82 (m, 2H), 1.82-1.74 (m, 2H).

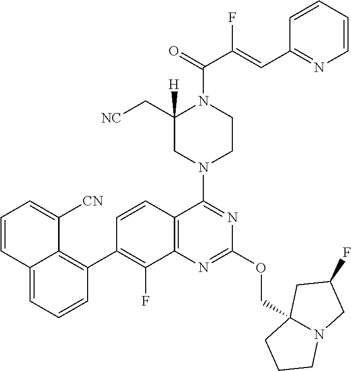 [SMILES: N#CC[C@H]1CN(c2nc(OC[C@@]34CCCN3C[C@H](F)C4)nc3c(F)c(-c4cccc5cccc(C#N)c45)ccc23)CCN1C(=O)/C(F)=C/c1ccccn1]

### Example 78

### 8-(4-((S)-3-(cyanomethyl)-4-((Z)-2-fluoro-3-(pyridin-2-yl)acryloyl)piperazin-1-yl)-8-fluoro-2-(((2R,7aS)-2-fluorotetrahydro-1H-pyrrolizin-7a(5H)-yl)methoxy)quinazolin-7-yl)-1-naphthonitrile

8-(4-((S)-3-(Cyanomethyl)piperazin-1-yl)-8-fluoro-2-(((2R,7aS)-2-fluorotetrahydro-1H-pyrrolizin-7a(5H)-yl)methoxy)quinazolin-7-yl)-1-naphthonitrile, HCl (12 mg, 0.019 mmol) and (Z)-2-fluoro-3-(pyridin-2-yl)acrylic acid (3.3 mg, 0.019 mmol) were combined as solids and dissolved in DMF (1.0 mL). 1-Methylimidazole (16 μL, 0.19 mmol) was added followed by chloro-N,N,N′,N′-tetramethylformamidinium hexafluorophosphate (5.5 mg, 0.019 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/8% 95:5 MeCN:H₂O with 10 mM AA→100% 95:5 MeCN:H₂O with 10 mM AA; λ=220 nm) to provide the desired product (4.6 mg, 0.006 mmol, 32% yield). LC/MS (ESI) m/z: [M+H]⁺ calcd for C₄₁H₃₆F₃N₈O₂ 729.3. found 729.2; ¹H NMR (500 MHz, DMSO-d₆) δ 8.66 (d, J=4.2 Hz, 1H), 8.47 (d, J=8.4 Hz, 1H), 8.27 (d, J=7.9 Hz, 1H), 8.17-8.13 (m, 1H), 8.00-7.89 (m, 2H), 7.85-7.67 (m, 4H), 7.42-7.36 (m, 2H), 6.66 (d, J=35 Hz, 1H), 5.34 (br s, 1H), 5.23 (br d, J=2.3 Hz, 1H), 4.39-4.29 (m, 2H), 4.29-4.20 (m, 1H), 4.20-3.99 (m, 4H), 3.65 (br s, 1H), 3.59 (br d, J=11.4 Hz, 1H), 3.54-3.45 (m, 1H), 3.24-3.21 (m, 1H), 3.19-3.03 (m, 1H), 2.97-2.79 (m, 2H), 2.57-2.52 (m, 1H), 2.20-1.99 (m, 2H), 1.89-1.73 (m, 2H), 1.04 (d, J=6.1 Hz, 1H).

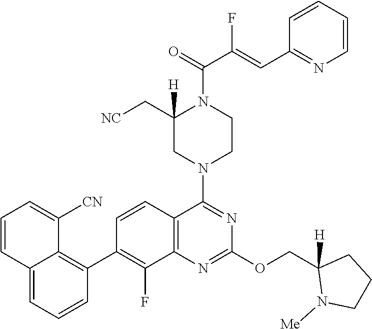 [SMILES: CN1CCC[C@H]1COc1nc(N2CCN(C(=O)/C(F)=C/c3ccccn3)[C@@H](CC#N)C2)c2ccc(-c3cccc4cccc(C#N)c34)c(F)c2n1]

### Example 79

### 8-(4-((S)-3-(cyanomethyl)-4-((Z)-2-fluoro-3-(pyridin-2-yl)acryloyl)piperazin-1-yl)-8-fluoro-2-(((S)-1-methylpyrrolidin-2-yl)methoxy)quinazolin-7-yl)-1-naphthonitrile

8-(4-((S)-3-(Cyanomethyl)piperazin-1-yl)-8-fluoro-2-(((S)-1-methylpyrrolidin-2-yl)methoxy)quinazolin-7-yl)-1-naphthonitrile (10 mg, 0.019 mmol) and (Z)-2-fluoro-3-(pyridin-2-yl)acrylic acid (3.3 mg, 0.019 mmol) were combined as solids and dissolved in DMF (1.0 mL). 1-Methylimidazole (16 μL, 0.19 mmol) was added followed by chloro-N,N,N′,N′-tetramethylformamidinium hexafluorophosphate (5.5 mg, 0.019 mmol). The reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/8% 95:5 MeCN:H₂O with 10 mM AA→100% 95:5 MeCN:H₂O with 10 mM AA; λ=220 nm) to provide the desired product (4.1 mg, 0.006 mmol, 30% yield). LC/MS (ESI) m/z: [M+H]⁺ calcd for C₃₉H₃₅F₂N₈O₂ 685.3. found 685.3; ¹H NMR (500 MHz, DMSO-d₆) δ 8.67 (br d, J=4.2 Hz, 1H), 8.48 (d, J=8.7 Hz, 1H), 8.29 (d, J=7.8 Hz, 1H), 8.15 (dd, J=7.2, 2.4 Hz, 1H), 8.04-7.88 (m, 2H), 7.88-7.73 (m, 2H), 7.71-7.60 (m, 1H), 7.49-7.32 (m, 2H), 7.24-7.03 (m, 1H), 6.67 (d, J=35 Hz, 1H), 5.16 (s, 1H), 4.75 (dt, J=12.5, 3.3 Hz, 1H), 4.68-4.56 (m, 1H), 4.47-4.35 (m, 2H), 4.32 (br d, J=14.5 Hz, 1H), 3.97 (br d, J=2.7 Hz, 1H), 3.93-3.80 (m, 2H), 3.77-3.60 (m, 1H), 3.41-3.22 (m, 1H), 3.21-3.05 (m, 2H), 2.98 (br s, 3H), 2.84 (br s, 1H), 2.36-2.19 (m, 1H), 2.14-2.02 (m, 1H), 2.00-1.86 (m, 2H).

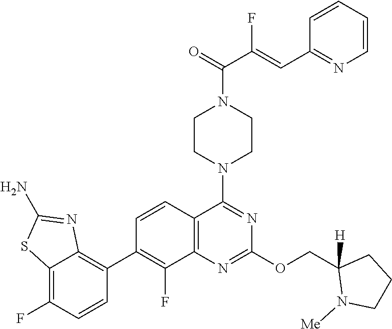 [SMILES: CN1CCC[C@H]1COc1nc(N2CCN(C(=O)/C(F)=C/c3ccccn3)CC2)c2ccc(-c3ccc(F)c4sc(N)nc34)c(F)c2n1]

### Example 80

### (Z)-1-((1R,5S)-3-(7-(2-amino-7-fluorobenzo[d]thiazol-4-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one

tert-Butyl (7-fluoro-4-(8-fluoro-4-((1R,5S)-8-((Z)-2-fluoro-3-(pyridin-2-yl)acryloyl)-3,8-diazabicyclo[3.2.1]octan-3-yl)-2-(((S)-1-methylpyrrolidin-2-yl)methoxy)quinazolin-7-yl)benzo[d]thiazol-2-yl)carbamate (10 mg, 0.013 mmol) was dissolved in dioxane (1.0 mL), and HCl solution (4.0 M in dioxane, 3 μL, 0.013 mmol) was added. The reaction mixture was stirred for 1.5 h, and was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (2.6 mg, 0.004 mmol, 30% yield). LC/MS (ESI) m/z: [M+H]⁺ calcd for C₃₅H₃₄F₃N₈O₂S 687.2. found 687.5; ¹H NMR (500 MHz, DMSO-d₆) δ 8.69-8.64 (m, 1H), 7.94-7.86 (m, 3H), 7.84-7.78 (m, 2H), 7.43-7.30 (m, 3H), 7.05 (t, J=8.8 Hz, 1H), 6.81 (d, J=37.8 Hz, 1H), 4.83-4.74 (m, 2H), 4.55-4.38 (m, 3H), 4.33-4.15 (m, 1H), 3.76-3.58 (m, 2H), 3.08-2.95 (m, 1H), 2.81-2.60 (m, 1H), 2.43 (br s, 3H), 2.33-2.15 (m, 1H), 2.08-1.94 (m, 2H), 1.88-1.83 (m, 2H), 1.76-1.58 (m, 4H).

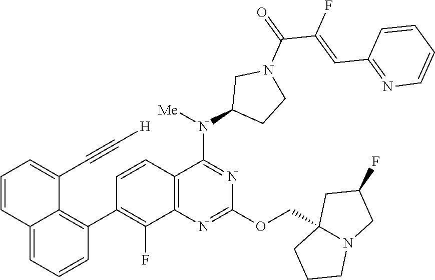 [SMILES: C#Cc1cccc2cccc(-c3ccc4c(N(C)[C@@H]5CCN(C(=O)/C(F)=C/c6ccccn6)C5)nc(OC[C@@]56CCCN5C[C@H](F)C6)nc4c3F)c12]

### Example 81

### (Z)-1-((R)-3-((7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(pyridin-2-yl)prop-2-en-1-one

7-(8-Ethynylnaphthalen-1-yl)-8-fluoro-2-(((2R,7aS)-2-fluorotetrahydro-1H-pyrrolizin-7a(5H)-yl)methoxy)-N-methyl-N-((S)-pyrrolidin-3-yl)quinazolin-4-amine, HCl salt (20 mg, 0.034 mmol and (Z)-2-fluoro-3-(pyridin-2-yl)acrylic acid (5.7 mg, 0.034 mmol) were combined as solids and dissolved in DMF (500 μL). 1-methylimidazole (27 μL, 0.34 mmol) was added followed by chloro-N,N,N′,N′-tetramethylformamidinium hexafluorophosphate (9.5 mg, 0.034 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 70% 5:95 MeCN:H₂O with 10 mM AA/30% 95:5 MeCN:H₂O with 10 mM AA→100% 95:5 MeCN:H₂O with 10 mM AA; λ=220 nm) to provide the desired product (13.8 mg, 0.020 mmol, 58% yield) as a brown solid. LC/MS (ESI) m/z: [M+H]⁺ calcd for C₄₁H₃₈F₃N₆O₂ 703.3. found 703.3; ¹H NMR (500 MHz, DMSO-d₆) δ 8.69-8.61 (m, 1H), 8.17-8.05 (m, 2H), 7.93-7.83 (m, 2H), 7.81-7.75 (m, 1H), 7.74-7.70 (m, 1H), 7.69-7.65 (m, 1H), 7.58-7.54 (m, 1H), 7.52-7.47 (m, 1H), 7.41-7.35 (m, 1H), 7.29-7.20 (m, 1H), 6.81 (d, J=38.2 Hz, 1H), 5.38-5.13 (m, 1H), 5.12-5.03 (m, 1H), 4.36-3.96 (m, 4H), 3.87-3.51 (m, 4H), 3.19-2.69 (m, 6H), 2.42-2.24 (m, 2H), 2.20-1.92 (m, 3H), 1.88-1.66 (m, 3H).

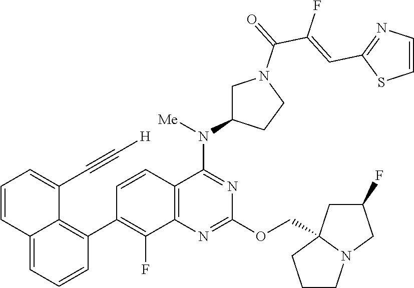 [SMILES: C#Cc1cccc2cccc(-c3ccc4c(N(C)[C@@H]5CCN(C(=O)/C(F)=C/c6nccs6)C5)nc(OC[C@@]56CCCN5C[C@H](F)C6)nc4c3F)c12]

### Example 82

### (Z)-1-((R)-3-((7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(thiazol-2-yl)prop-2-en-1-one

7-(8-Ethynylnaphthalen-1-yl)-8-fluoro-2-(((2R,7aS)-2-fluorotetrahydro-1H-pyrrolizin-7a(5H)-yl)methoxy)-N-methyl-N-((S)-pyrrolidin-3-yl)quinazolin-4-amine, HCl salt (20 mg, 0.034 mmol) and (Z)-2-fluoro-3-(pyridin-2-yl)acrylic acid (5.9 mg, 0.034 mmol) were combined as solids and dissolved in DMF (500 μL). 1-methylimidazole (27 μL, 0.34 mmol) was added followed by chloro-N,N,N′,N′-tetramethylformamidinium hexafluorophosphate (9.5 mg, 0.034 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 70% 5:95 MeCN:H₂O with 10 mM AA/30% 95:5 MeCN:H₂O with 10 mM AA→100% 95:5 MeCN:H₂O with 10 mM AA; λ=220 nm) to provide the desired product (18.6 mg, 0.020 mmol, 58% yield) as a brown solid. LC/MS (ESI) m/z: [M+H]⁺ calcd for C₃₉H₃₆F₃N₆O₂S 709.2. found 709.2; ¹H NMR (500 MHz, DMSO-d₆) δ 8.15-8.08 (m, 2H), 8.04-7.96 (m, 2H), 7.91-7.85 (m, 1H), 7.73-7.65 (m, 2H), 7.59-7.53 (m, 1H), 7.52-7.46 (m, 1H), 7.24 (br s, 1H), 7.20 (d, J=36.7 Hz, 1H), 5.36-5.15 (m, 1H), 5.15-5.03 (m, 1H), 4.40-3.91 (m, 4H), 3.88-3.50 (m, 4H), 3.17-2.63 (m, 6H), 2.43-2.20 (m, 2H), 2.18-1.93 (m, 3H), 1.86-1.67 (m, 3H).

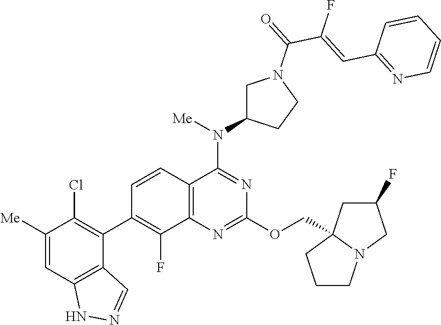 [SMILES: Cc1cc2[nH]ncc2c(-c2ccc3c(N(C)[C@@H]4CCN(C(=O)/C(F)=C/c5ccccn5)C4)nc(OC[C@@]45CCCN4C[C@H](F)C5)nc3c2F)c1Cl]

### Example 83

### (Z)-1-((3R)-3-((7-(5-chloro-6-methyl-1H-indazol-4-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(pyridin-2-yl)prop-2-en-1-one

7-(5-Chloro-6-methyl-1H-indazol-4-yl)-8-fluoro-2-(((2R,7aS)-2-fluorotetrahydro-1H-pyrrolizin-7a(5H)-yl)methoxy)-N-methyl-N-((S)-pyrrolidin-3-yl)quinazolin-4-amine, HCl salt (20 mg, 0.020 mmol) and (Z)-2-fluoro-3-(pyridin-2-yl)acrylic acid (3.3 mg, 0.020 mmol) were combined as solids and dissolved in DMF (500 μL). 1-methylimidazole (16 μL, 0.20 mmol) was added followed by chloro-N,N,N′,N′-tetramethylformamidinium hexafluorophosphate (5.6 mg, 0.020 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 95% 5:95 MeCN:H₂O with 0.1% TFA/5% 95:5 MeCN:H₂O with 0.1% TFA→100% 95:5 MeCN:H₂O with 0.1% TFA; λ=220 nm) to provide the desired product as its bis TFA salt (17.8 mg, 0.019 mmol, 95% yield). LC/MS (ESI) m/z: [M+H]⁺ calcd for C₃₇H₃₇ClF₃N₈O₂ 717.3. found 717.4; ¹H NMR (500 MHz, DMSO-d₆) δ 10.93 (br s, 1H), 8.71-8.61 (m, 1H), 8.10 (br d, J=8.8 Hz, 1H), 7.97-7.88 (m, 1H), 7.85-7.77 (m, 1H), 7.68 (s, 1H), 7.64-7.57 (m, 1H), 7.45-7.33 (m, 2H), 6.83 (d, J=37.8 Hz, 1H), 5.56 (d, J=53.3 Hz, 1H), 5.27-5.10 (m, 1H), 4.70-4.56 (m, 2H), 4.39-4.21 (m, 1H), 4.13-3.96 (m, 2H), 3.91-3.83 (m, 4H), 3.35-3.19 (m, 1H), 2.67-2.54 (m, 6H), 2.43-2.25 (m, 3H), 2.23-1.92 (m, 3H).

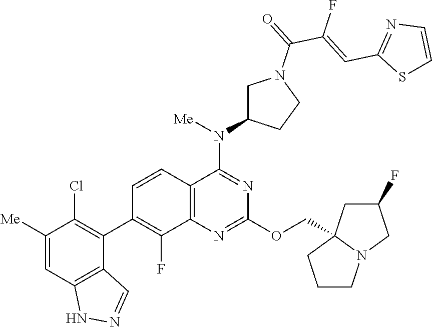 [SMILES: Cc1cc2[nH]ncc2c(-c2ccc3c(N(C)[C@@H]4CCN(C(=O)/C(F)=C/c5nccs5)C4)nc(OC[C@@]45CCCN4C[C@H](F)C5)nc3c2F)c1Cl]

### Example 84

### (Z)-1-((3R)-3-((7-(5-chloro-6-methyl-1H-indazol-4-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(thiazol-2-yl)prop-2-en-1-one

7-(5-Chloro-6-methyl-1H-indazol-4-yl)-8-fluoro-2-(((2R,7aS)-2-fluorotetrahydro-1H-pyrrolizin-7a(5H)-yl)methoxy)-N-methyl-N-((S)-pyrrolidin-3-yl)quinazolin-4-amine, HCl salt (20 mg, 0.020 mmol) and (Z)-2-fluoro-3-(thiazol-2-yl)acrylic acid (3.3 mg, 0.020 mmol) were combined as solids and dissolved in DMF (500 μL). 1-methylimidazole (16 μL, 0.20 mmol) was added followed by chloro-N,N,N′,N′-tetramethylformamidinium hexafluorophosphate (5.6 mg, 0.020 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 95% 5:95 MeCN:H₂O with 0.1% TFA/5% 95:5 MeCN:H₂O with 0.1% TFA→100% 95:5 MeCN:H₂O with 0.1% TFA; λ=220 nm) to provide the desired product as its bis TFA salt (15.9 mg, 0.017 mmol, 84% yield). LC/MS (ESI) m/z: [M+H]⁺ calcd for C₃₅H₃₅ClF₃N₈O₂S 723.2. found 723.3; ¹H NMR (500 MHz, DMSO-d₆) δ 10.97 (s, 1H), 8.15-8.06 (m, 1H), 8.05-7.96 (m, 2H), 7.73-7.57 (m, 2H), 7.42-7.33 (m, 1H), 7.21 (d, J=38.9 Hz, 1H), 5.53 (d, J=54.8 Hz, 1H), 5.22-5.11 (m, 1H), 4.69-4.55 (m, 2H), 4.38-4.25 (m, 1H), 4.15-3.98 (m, 4H), 3.84-3.63 (m, 6H), 3.61-3.49 (m, 5H), 2.40-2.25 (m, 3H), 2.21-1.93 (m, 3H).

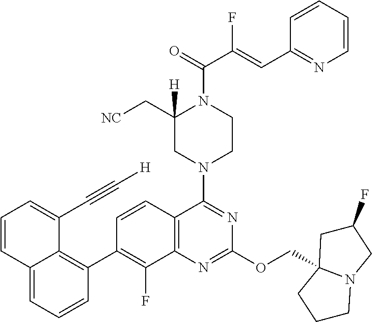 [SMILES: C#Cc1cccc2cccc(-c3ccc4c(N5CCN(C(=O)/C(F)=C/c6ccccn6)[C@@H](CC#N)C5)nc(OC[C@@]56CCCN5C[C@H](F)C6)nc4c3F)c12]

### Example 85

### 2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyridin-2-yl)acryloyl)piperazin-2-yl)acetonitrile

2-((S)-4-(7-(8-Ethynylnaphthalen-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile, HCl salt (10 mg, 0.016 mmol) and (Z)-2-fluoro-3-(pyridin-2-yl)acrylic acid (2.7 mg, 0.016 mmol) were combined as solids and dissolved in DMF (500 μL). 1-methylimidazole (13 μL, 0.16 mmol) was added followed by chloro-N,N,N′,N′-tetramethylformamidinium hexafluorophosphate (4.6 mg, 0.016 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 68% 5:95 MeCN:H₂O with 10 mM AA/32% 95:5 MeCN:H₂O with 10 mM AA→100% 95:5 MeCN:H₂O with 10 mM AA; λ=220 nm) to provide the desired product (1.8 mg, 0.002 mmol, 15% yield). LC/MS (ESI) m/z: [M+H]⁺ calcd for C₄₂H₃₇F₃N₇O₂ 728.3. found 728.3; ¹H NMR (500 MHz, DMSO-d₆) δ 8.76-8.56 (m, 1H), 8.16-8.09 (m, 2H), 7.94-7.78 (m, 3H), 7.75-7.66 (m, 2H), 7.61-7.54 (m, 1H), 7.52-7.47 (m, 1H), 7.42-7.34 (m, 1H), 7.32-7.25 (m, 1H), 6.64 (d, J=38.3 Hz, 1H), 5.27 (d, J=54.2 Hz, 1H), 5.08-4.85 (m, 1H), 4.38-4.21 (m, 4H), 4.15-3.98 (m, 4H), 3.66-3.53 (m, 4H), 3.15-2.99 (m, 2H), 2.89-2.79 (m, 1H), 2.17-1.97 (m, 3H), 1.92-1.76 (m, 3H).

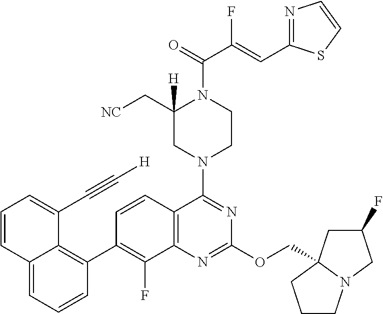 [SMILES: C#Cc1cccc2cccc(-c3ccc4c(N5CCN(C(=O)/C(F)=C/c6nccs6)[C@@H](CC#N)C5)nc(OC[C@@]56CCCN5C[C@H](F)C6)nc4c3F)c12]

### Example 86

### 2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(thiazol-2-yl)acryloyl)piperazin-2-yl)acetonitrile

2-((S)-4-(7-(8-Ethynylnaphthalen-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile, HCl salt (10 mg, 0.016 mmol) and (Z)-2-fluoro-3-(thiazol-2-yl)acrylic acid (2.8 mg, 0.016 mmol) were combined as solids and dissolved in DMF (500 μL). 1-methylimidazole (13 μL, 0.16 mmol) was added followed by chloro-N,N,N′,N′-tetramethylformamidinium hexafluorophosphate (4.6 mg, 0.016 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 0.1% TFA/23% 95:5 MeCN:H₂O with 0.1% TFA→100% 95:5 MeCN:H₂O with 0.1% TFA; λ=220 nm) to provide the desired product as its bis TFA salt (4.6 mg, 0.005 mmol, 30% yield). LC/MS (ESI) m/z: [M+H]⁺ calcd for C₄₀H₃₅F₃N₇O₂S 734.3. found 734.4; ¹H NMR (500 MHz, DMSO-d₆) δ 8.19-8.08 (m, 2H), 8.07-7.99 (m, 2H), 7.94-7.86 (m, 1H), 7.76-7.67 (m, 2H), 7.60-7.48 (m, 2H), 7.40-7.34 (m, 1H), 7.11 (d, J=37.4 Hz, 1H), 5.55 (d, J=53.7 Hz, 1H), 5.04-4.51 (m, 2H), 4.47-4.32 (m, 2H), 3.94-3.72 (m, 4H), 3.48-3.28 (m, 4H), 3.20-3.08 (m, 4H), 2.66-2.55 (m, 2H), 1.97 (s, 4H).

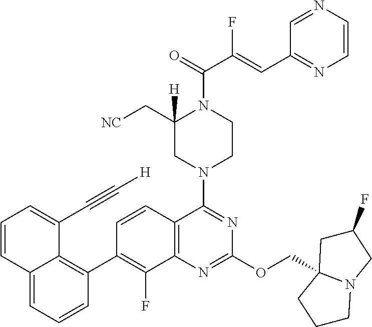 [SMILES: C#Cc1cccc2cccc(-c3ccc4c(N5CCN(C(=O)/C(F)=C/c6cnccn6)[C@@H](CC#N)C5)nc(OC[C@@]56CCCN5C[C@H](F)C6)nc4c3F)c12]

### Example 87

### 2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyrazin-2-yl)acryloyl)piperazin-2-yl)acetonitrile

2-((S)-4-(7-(8-Ethynylnaphthalen-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile, HCl salt (10 mg, 0.016 mmol) and (Z)-2-fluoro-3-(pyrazin-2-yl)acrylic acid (2.7 mg, 0.016 mmol) were combined as solids and dissolved in DMF (500 μL). 1-methylimidazole (13 μL, 0.16 mmol) was added followed by chloro-N,N,N′,N′-tetramethylformamidinium hexafluorophosphate (4.6 mg, 0.016 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 68% 5:95 MeCN:H₂O with 10 mM AA/32% 95:5 MeCN:H₂O with 10 mM AA→100% 95:5 MeCN:H₂O with 10 mM AA; λ=220 nm) to provide the desired product (2.2 mg, 0.003 mmol, 19% yield). LC/MS (ESI) m/z: [M+H]⁺ calcd for C₄₁H₃₆F₃N₈O₂ 729.3. found 729.2; ¹H NMR (500 MHz, DMSO-d₆) δ 8.98 (s, 1H), 8.74 (d, J=2.0 Hz, 1H), 8.62 (d, J=2.0 Hz, 1H), 8.20-8.07 (m, 2H), 7.89-7.77 (m, 1H), 7.74-7.64 (m, 2H), 7.59-7.48 (m, 2H), 7.29 (br t, J=6.6 Hz, 1H), 6.73 (d, J=39.3 Hz, 1H), 5.27 (d, J=55.1 Hz, 1H), 5.16-4.88 (m, 1H), 4.55-3.97 (m, 7H), 3.66-3.54 (m, 4H), 3.22-2.97 (m, 3H), 2.88-2.77 (m, 1H), 2.19-1.97 (m, 3H), 1.88-1.70 (m, 3H).

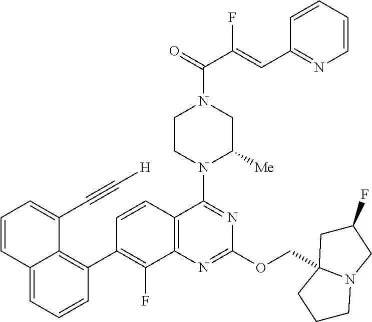 [SMILES: C#Cc1cccc2cccc(-c3ccc4c(N5CCN(C(=O)/C(F)=C/c6ccccn6)C[C@@H]5C)nc(OC[C@@]56CCCN5C[C@H](F)C6)nc4c3F)c12]

### Example 88

### (Z)-1-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-3-methylpiperazin-1-yl)-2-fluoro-3-(pyridin-2-yl)prop-2-en-1-one

7-(8-Ethynylnaphthalen-1-yl)-8-fluoro-2-(((2R,7aS)-2-fluorotetrahydro-1H-pyrrolizin-7a(5H)-yl)methoxy)-4-((S)-2-methylpiperazin-1-yl)quinazoline, HCl salt (20 mg, 0.034 mmol) and (Z)-2-fluoro-3-(pyridin-2-yl)acrylic acid (5.7 mg, 0.034 mmol) were combined as solids and dissolved in DMF (500 μL). 1-methylimidazole (27 μL, 0.34 mmol) was added followed by chloro-N,N,N′,N′-tetramethylformamidinium hexafluorophosphate (9.5 mg, 0.034 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 65% 5:95 MeCN:H₂O with 10 mM AA/5% 35:5 MeCN:H₂O with 10 mM AA→100% 95:5 MeCN:H₂O with 10 mM AA; λ=220 nm) to provide the desired product (7.3 mg, 0.010 mmol, 30% yield). LC/MS (ESI) m/z: [M+H]⁺ calcd for C₄₁H₃₈F₃N₆O₂ 703.3. found 703.4; ¹H NMR (500 MHz, DMSO-d₆) δ 8.65 (d, J=4.5 Hz, 1H), 8.16-8.06 (m, 2H), 7.95-7.85 (m, 1H), 7.81-7.77 (m, 1H), 7.76-7.71 (m, 2H), 7.70-7.65 (m, 1H), 7.60-7.54 (m, 1H), 7.53-7.47 (m, 1H), 7.40-7.35 (m, 1H), 7.32-7.23 (m, 1H), 6.59 (d, J=38.8 Hz, 1H), 5.26 (d, J=53.9 Hz, 1H), 4.95-4.66 (m, 1H), 4.32-3.92 (m, 6H), 3.77-3.58 (m, 2H), 3.07 (br d, J=13.2 Hz, 3H), 2.88-2.79 (m, 1H), 2.18-1.94 (m, 3H), 1.86-1.65 (m, 3H), 1.41-1.36 (m, 3H).

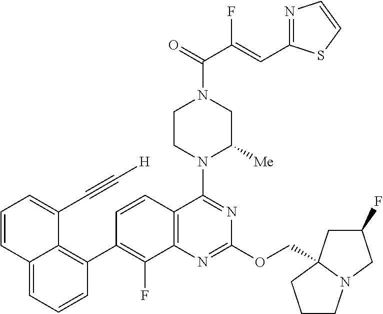 [SMILES: C#Cc1cccc2cccc(-c3ccc4c(N5CCN(C(=O)/C(F)=C/c6nccs6)C[C@@H]5C)nc(OC[C@@]56CCCN5C[C@H](F)C6)nc4c3F)c12]

### Example 89

### (Z)-1-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-3-methylpiperazin-1-yl)-2-fluoro-3-(thiazol-2-yl)prop-2-en-1-one

7-(8-Ethynylnaphthalen-1-yl)-8-fluoro-2-(((2R,7aS)-2-fluorotetrahydro-1H-pyrrolizin-7a(5H)-yl)methoxy)-4-((S)-2-methylpiperazin-1-yl)quinazoline, HCl salt (20 mg, 0.034 mmol) and (Z)-2-fluoro-3-(thiazol-2-yl)acrylic acid (5.9 mg, 0.034 mmol) were combined as solids and dissolved in DMF (500 μL). 1-methylimidazole (27 μL, 0.34 mmol) was added followed by chloro-N,N,N′,N′-tetramethylformamidinium hexafluorophosphate (9.5 mg, 0.034 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 65% 5:95 MeCN:H₂O with 10 mM AA/5% 35:5 MeCN:H₂O with 10 mM AA→100% 95:5 MeCN:H₂O with 10 mM AA; λ=220 nm) to provide the desired product (7.2 mg, 0.010 mmol, 29% yield). LC/MS (ESI) m/z: [M+H]⁺ calcd for C₃₉H₃₆F₃N₆O₂S 709.3. found 709.2; ¹H NMR (500 MHz, DMSO-d₆) δ 8.15-8.09 (m, 2H), 8.05-7.97 (m, 2H), 7.79-7.70 (m, 2H), 7.70-7.65 (m, 1H), 7.56 (t, J=7.7 Hz, 1H), 7.52-7.47 (m, 1H), 7.31-7.25 (m, 1H), 7.08 (d, J=37.7 Hz, 1H), 5.26 (d, J=53.2 Hz, 1H), 4.91-4.63 (m, 1H), 4.31-3.95 (m, 5H), 3.84-3.51 (m, 4H), 3.13-3.06 (m, 3H), 2.87-2.80 (m, 1H), 2.20-1.95 (m, 3H), 1.85-1.66 (m, 3H), 1.43-1.34 (m, 3H).

### Biological Activity

### Example 90

### KRAS^{G12C} RAF Disruption Assay

This is a functional assay that measures activity of compounds against KRAS-G12C^{(ON)}, i.e., the active form of KRAS G12C. Recombinant GMPPNP-loaded KRAS G12C (5 nM) was treated with compound at room temperature for 20 minutes in assay buffer (50 mM Tris pH 7.5, 100 mM NaCl, 1 mM MgCl₂, 1 mM DTT, 100 μg/ml BSA). Recombinant GST-RAF1 RBD (9 nM) was added, and the reaction mixture was incubated for 20 minutes. SA-Tb (0.25 nM) was added, and the reaction mixture was incubated for 3 hours. HTRF signal was measured (PerkinElmer Envision), the signal ratio (λₑₘ 520/λₑₘ 495) was calculated, and IC₅₀ values were calculated from the dose-response curve.

The IC₅₀ values for compounds described herein are shown in Table 1.

TABLE 1

| | KRASG12C RAF | |
|---|---|---|
| | Disruption Assay | |
| Example No. | (IC₅₀) | |
| 1 | 49.0 | μM |
| 2 | 61.3 | μM |
| 3 | 5.5 | μM |
| 4 | 5.8 | μM |
| 5 | 2.7 | μM |
| 6 | 4.9 | μM |
| 7 | 2.0 | μM |
| 8 | 9.6 | μM |
| 9 | 1.9 | μM |
| 10 | 93.0 | μM |
| 11 | 5.3 | μM |
| 12 | 9.20 | μM |
| 13 | 1.1 | μM |
| 14 | 2.1 | μM |
| 15, Isomer 1 | 93.8 | μM |
| 15, Isomer 2 | 47.4 | μM |
| 16 | 4.2 | μM |
| 17 | 5.8 | μM |
| 18 | 430 | μM |
| 19 | 3.2 | μM |
| 20, Isomer 1 | 16.5 | μM |
| 20, Isomer 2 | >100 | μM |
| 21 | 400 | nM |
| 22 | 260 | nM |
| 23 | 16 | nM |
| 24 | 22 | nM |
| 25, Isomer 1 | 14.1 | μM |
| 25, Isomer 2 | 67.6 | μM |
| 26, Isomer 1 | 3.37 | μM |
| 26, Isomer 2 | 55.2 | μM |
| 27 | 6.4 | nM |
| 28 | 350 | nM |
| 29 | 180 | nM |
| 30 | 21.3 | μM |
| 31 | 10 | nM |
| 32 | 290 | nM |
| 33 | 76 | nM |
| 34 | 27 | nM |
| 35 | 290 | nM |
| 36 | 25 | nM |
| 37 | 110 | nM |
| 38 | 130 | nM |
| 39 | 23 | nM |
| 40 | 11 | nM |
| 41 | 46 | nM |
| 42 | 88 | nM |
| 43 | 190 | nM |
| 44 | 350 | nM |
| 45 | 48 | nM |
| 46 | 23 | nM |
| 47 | 1.33 | μM |
| 48 | 2.59 | μM |
| 49 | 2.26 | μM |
| 50 | 100 | nM |
| 51 | 160 | nM |
| 52 | 450 | nM |
| 53 | 70 | nM |
| 54 | 200 | nM |
| 55 | 1000 | nM |
| 56 | 270 | nM |
| 57 | 130 | nM |
| 58 | 2400 | nM |
| 59 | 260 | nM |
| 60 | 170 | nM |
| 61 | 2800 | nM |
| 62 | 3100 | nM |
| 63 | 9800 | nM |
| 64 | 61000 | nM |
| 65 | 2100 | nM |
| 66 | 3200 | nM |
| 67 | 4700 | nM |
| 68 | 20400 | nM |
| 69 | 2600 | nM |
| 70 | 7900 | nM |
| 71 | 9000 | nM |
| 72 | 2 | nM |
| 73 | 710 | nM |
| 74 | 1500 | nM |
| 75 | 60 | nM |
| 76 | 50 | nM |
| 77 | 50 | nM |
| 78 | 240 | nM |
| 79 | 1600 | nM |
| 80 | 550 | nM |
| 81 | 20 | nM |
| 82 | 40 | nM |
| 83 | 22 | nM |
| 84 | 98 | nM |
| 85 | 25 | nM |
| 86 | 44 | nM |
| 87 | 42 | nM |
| 88 | 43 | nM |
| 89 | 380 | nM |

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

All of the references cited herein are incorporated herein by reference in their entireties.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A compound of formula (I): 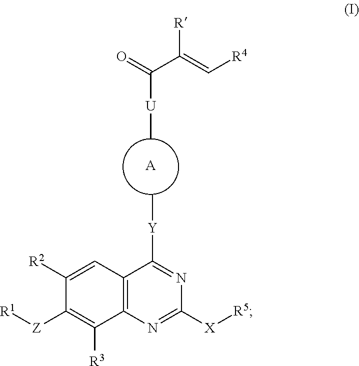
or a pharmaceutically acceptable salt thereof, wherein:
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkoxy, haloC₁-C₃alkyl, hydroxy, and hydroxyC₁-C₃alkyl;
R² and R³ are independently selected from hydrogen, C₁-C₃alkoxy, C₁-C₃alkyl, cyano, halo, haloC₁-C₃alkyl, —C(O)NH₂, —C(O)NH(C₁-C₃alkyl), —C(O)N(C₁-C₃alkyl)₂, and hydroxy;
U is a bond, CH₂NH, or NH;
Y is a bond, O, NR^{g}(CR^{e}R^{f})ₘ, NR^{f}, or CR^{e}R^{f}, wherein m is 1, 2, or 3, and wherein R^{e}, R^{f}, and R^{g} are independently hydrogen or C₁-C₃alkyl;
A is a four- to ten-membered monocyclic or a bicyclic or tricyclic bridged, fused, or spirocyclic saturated, unsaturated, or partially unsaturated ring system optionally containing one or two heteroatoms independently selected from nitrogen, oxygen, and sulfur, wherein the ring system is optionally substituted with one, two, or three groups independently selected from C₁-C₃alkoxy, C₁-C₃alkoxyC₁-C₃alkyl, C₁-C₃alkyl, amidoC₁-C₃alkyl, cyano, cyanoC₁-C₃alkyl, halo, haloC₁-C₃alkyl, amino, aminoC₁-C₃alkyl, hydroxy, hydroxyC₁-C₃alkyl, and oxo; provided that when Y is a bond, A contains at least one nitrogen atom;
R′ is halo;
R⁴ is an aryl or heteroaryl ring optionally substituted with one, two, or three substituents independently selected from C₂-C₄alkenyl, C₁-C₃alkyl, cyano, cyanoC₁-C₃alkyl, halo, haloC₁-C₃alkoxy, haloC₁-C₃alkyl, nitro, and oxo;
X is O or NR¹⁶, wherein R¹⁶ is hydrogen or C₁-C₃alkyl;
R⁵ is selected from hydrogen, C₁-C₆alkoxyC₁-C₆alkyl, C₁-C₆alkyl, aryl, arylC₁-C₆alkyl, carboxyC₁-C₆alkyl, C₃-C₆cycloalkyl, C₃-C₆cycloalkylC₁-C₆alkyl, di(C₁-C₃alkyl)aminoC₂-C₆alkyl, haloC₁-C₆alkyl, heteroaryl, heteroarylC₁-C₆alkyl, heterocyclyl, heterocyclylC₁-C₆alkyl, hydroxyC₁-C₆alkyl, NR^{a}R^{b}—C(O)—C₁-C₆alkyl), NR^{a}R^{b}C₁-C₆alkyl, wherein the aryl, the aryl part of the arylC₁-C₆alkyl, the C₃-C₆cycloalkyl, the cycloalkyl part of the C₃-C₆cycloalkylC₁-C₆alkyl, the heteroaryl, the heteroaryl part of the heteroarylC₁-C₆alkyl, the heterocyclyl, the heterocyclyl part of the heterocyclylC₁-C₆alkyl, are optionally substituted with one, two, three, or four groups independently selected from C₁-C₃alkoxy, C₁-C₃alkoxyC₁-C₃alkyl, C₁-C₃alkyl, (C₁-C₆alkyl)amino, (C₁-C₆alkyl)aminoC₁-C₃alkyl, amino, aminoC₁-C₃alkyl, carboxy, cyano, di(C₁-C₆alkyl)amino, di(C₁-C₆alkyl)aminoC₁-C₃alkyl, halo, haloC₁-C₃alkoxy, haloC₁-C₃alkyl, heterocyclyl, heterocyclylC₁-C₃alkyl, hydroxy, hydroxyC₁-C₃alkyl, nitro, and oxo; wherein the heterocyclyl and the heterocyclyl part of the heterocyclylC₁-C₃alkyl is further optionally substituted with one, two, or three groups independently selected from C₁-C₃alkoxy, C₁-C₃alkyl, halo, and haloC₁-C₃alkyl; or
R⁵ and R¹⁶, together with the nitrogen atom to which they are attached, form a heterocyclic group optionally substituted with one, two, three, four, or five groups independently selected from one, two, three, or four groups independently selected from C₁-C₃alkoxy, C₁-C₃alkoxyC₁-C₃alkyl, C₁-C₃alkyl, amino, aminoC₁-C₃alkyl, hydroxy, and hydroxyC₁-C₃alkyl; and
one of R^{a} and R^{b} is selected from hydrogen and C₁-C₃alkyl and the other is selected from hydrogen, C₁-C₃alkyl, C₁-C₃alkoxycarbonyl, C₁-C₃alkylcarbonyl, arylC₁-C₆alkyl, C₃-C₆cycloalkyl, and C₃-C₆cycloalkylC₁-C₆alkyl.

2. The compound of claim 1 wherein R⁴ is a five- or six-membered aromatic ring optionally containing one, two, or three heteroatoms independently selected from nitrogen, oxygen, and sulfur.

3. The compound of claim 1 or 2, or a pharmaceutically acceptable salt thereof, wherein Y is a bond.

4. The compound of claim 1 or 2, or a pharmaceutically acceptable salt thereof, wherein Y is NH.

5. The compound of claim 1 or 2, or a pharmaceutically acceptable salt thereof, wherein Y is NCH₃.

6. The compound of any one of claims 1 to 5, or a pharmaceutically acceptable salt thereof, wherein A is a five- to ten-membered monocyclic or a bicyclic or tricyclic bridged, fused, or spirocyclic saturated ring system containing one or two nitrogen atoms.

7. The compound of any one of claims 1 to 3 or 6, or a pharmaceutically acceptable salt thereof, wherein A-U is selected from the group consisting of: 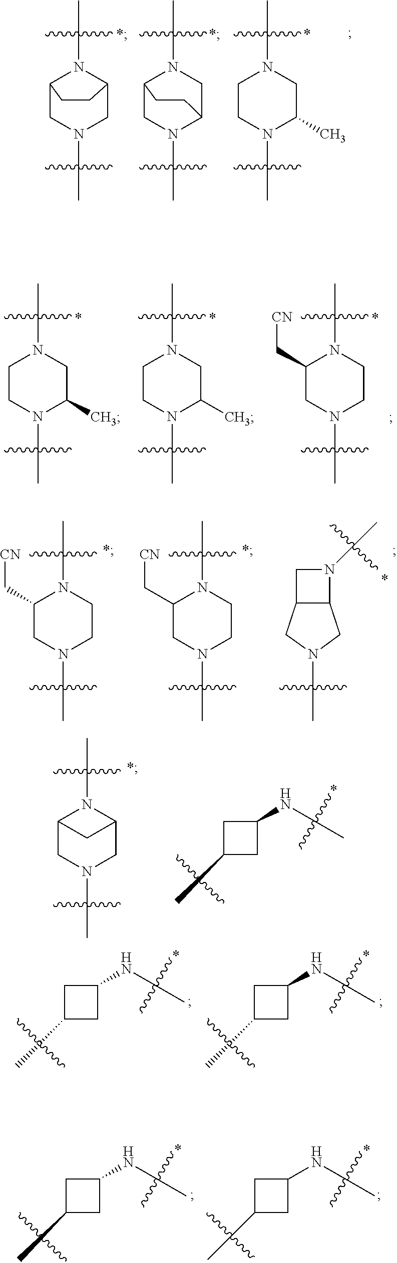 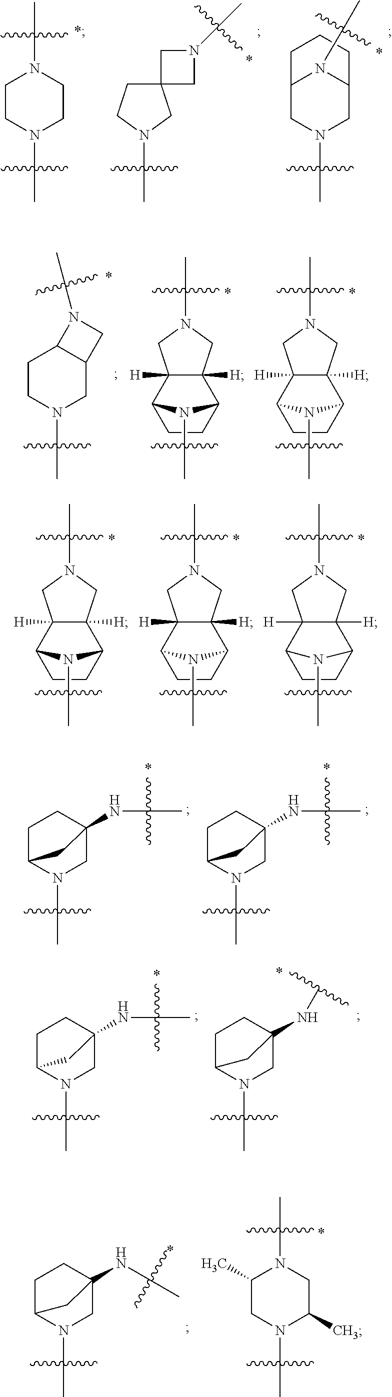 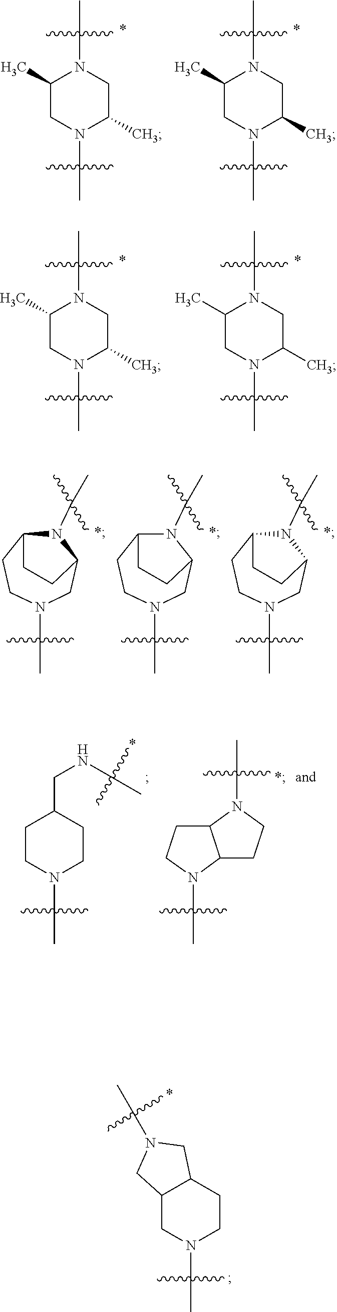
wherein [symbol]
represents the point of attachment to the carbonyl group; and [symbol]
represents the point of attachment to Y.

8. A compound of any one of claims 1, 2, 4, or 5, or a pharmaceutically acceptable salt thereof, wherein Y-A-U is selected from the group consisting of: 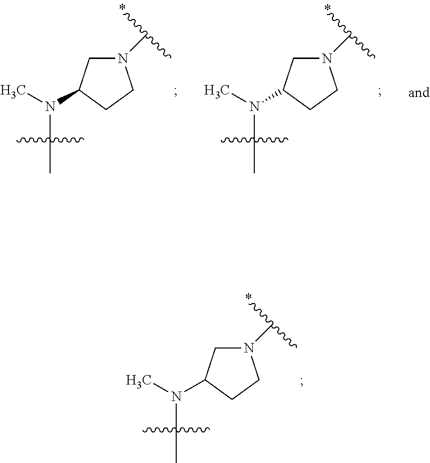
wherein: [symbol]
represents the point of attachment to the carbonyl group; and [symbol]
represents the point of attachment to the quinazoline ring.

9. The compound of any one of claims 1 to 8, or a pharmaceutically acceptable salt thereof, wherein R² is halo.

10. The compound of any one of claims 1 to 9, or a pharmaceutically acceptable salt thereof, wherein R³ is halo.

11. The compound of any one of claims 1 to 10, or a pharmaceutically acceptable salt thereof, wherein R⁴ is selected from isothiazolyl, pyridinyl, pyrimidinyl, and thiazolyl.

12. The compound of any one of claims 1 to 11, or a pharmaceutically acceptable salt thereof, wherein X is O.

13. The compound of any one of claims 1 to 12, or a pharmaceutically acceptable salt thereof, wherein R⁵ is selected from the group consisting of: 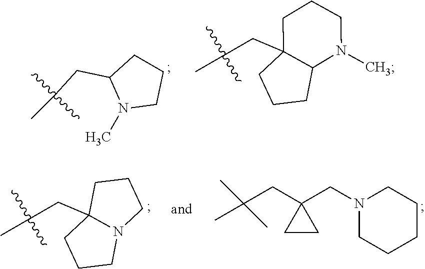
wherein each ring is optionally substituted with 1, 2, or 3 groups independently selected from C₁-C₃alkoxy, C₁-C₃alkoxyC₁-C₃alkyl, C₁-C₃alkyl, benzyl, halo, haloC₁-C₃alkyl, hydroxy, hydroxyC₁-C₃alkyl, and oxo.

14. The compound of any one of claims 1 to 12, or a pharmaceutically acceptable salt thereof, wherein R⁵ is —(C₁-C₃alkyl)-R⁶, wherein R⁶ is a three- to five-membered monocyclic ring system, an eight- or nine-membered bicyclic fused saturated ring system, or a ten-membered tricyclic saturated ring system, wherein each ring system optionally contains one nitrogen atom, and wherein each ring system is optionally substituted with one or two groups independently selected from C₁-C₃alkyl, halo, and (4- to 6-membered heterocyclyl)C₁-C₃alkyl; wherein the heterocyclyl part of the (4- to 6-membered heterocyclyl)C₁-C₃alkyl is further optionally substituted with a halo group.

15. The compound of any one of claims 1 to 12, or a pharmaceutically acceptable salt thereof, wherein R⁵ is 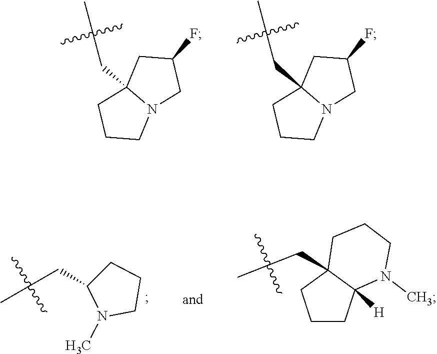
and [symbol] represents the point of attachment to X.

16. The compound of any one of claims 1 to 12, or a pharmaceutically acceptable salt thereof, wherein R⁵ is 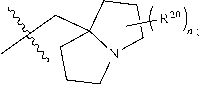
wherein;
n is 0, 1, or 2;
each R²⁰ is halo; and [symbol]
represents the point of attachment to X.

17. The compound of any one of claims 1 to 16, or a pharmaceutically acceptable salt thereof, wherein Z is a bond.

18. The compound of any one of claims 1 to 17, wherein R¹ is a monocyclic heteroaryl ring containing one, two, or three nitrogen atoms, wherein the ring is optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, hydroxy, and hydroxyC₁-C₃alkyl.

19. The compound of any one of claims 1 to 18, wherein R¹ is 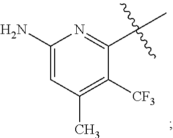
wherein [symbol] denotes the point of attachment to the parent molecular moiety.

20. The compound of any one of claims 1 to 17, or a pharmaceutically acceptable salt thereof, wherein R¹ is C₆-C₁₀aryl optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, hydroxy, and hydroxyC₁-C₃alkyl.

21. The compound of any one of claims 1 to 17 and 20, or a pharmaceutically acceptable salt thereof, wherein R¹ is naphtyl substituted with one, two, three, four, or five substituents independently selected from C₁-C₃alkyl, C₂-C₄alkynyl, halo, and hydroxy.

22. The compound of any one of claims 1 to 17, 20, and 21, or a pharmaceutically acceptable salt thereof, wherein R¹ is naphthyl, wherein the naphthyl is substituted with one, two, or three groups independently selected from C₂-C₄alkynyl, halo, and hydroxy.

23. The compound of any one of claims 1 to 17, and 20 to 22, or a pharmaceutically acceptable salt thereof, wherein R¹ is 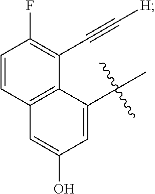
wherein [symbol] denotes the point of attachment to the parent molecular moiety.

24. The compound of any one of claims 1 to 23, or a pharmaceutically acceptable salt thereof, wherein R′ is fluoro.

25. The compound of any one of claims 1 to 23, or a pharmaceutically acceptable salt thereof, wherein R′ is chloro.

26. A compound selected from the group consisting of: 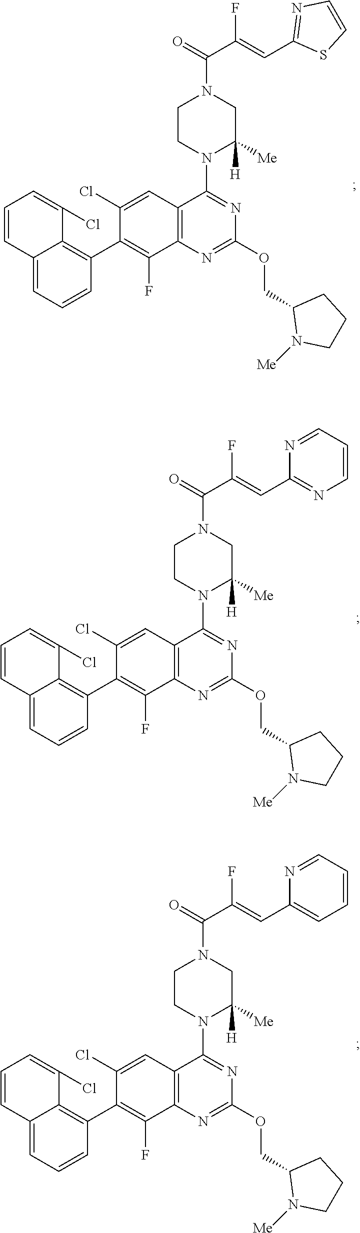 [SMILES: C[C@H]1CN(C(=O)/C(F)=C/c2nccs2)CCN1c1nc(OC[C@@H]2CCCN2C)nc2c(F)c(-c3cccc4cccc(Cl)c34)c(Cl)cc12 ; C[C@H]1CN(C(=O)/C(F)=C/c2ncccn2)CCN1c1nc(OC[C@@H]2CCCN2C)nc2c(F)c(-c3cccc4cccc(Cl)c34)c(Cl)cc12 ; C[C@H]1CN(C(=O)/C(F)=C/c2ccccn2)CCN1c1nc(OC[C@@H]2CCCN2C)nc2c(F)c(-c3cccc4cccc(Cl)c34)c(Cl)cc12] 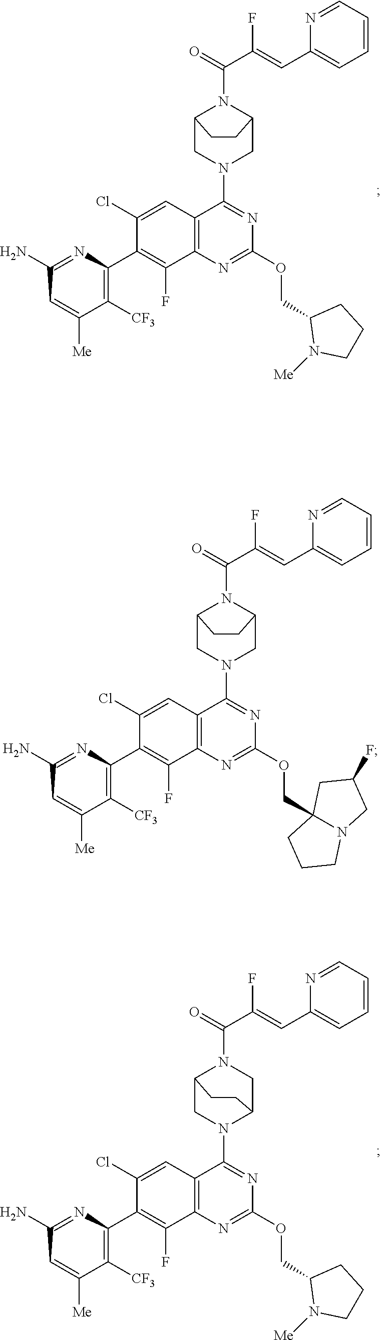 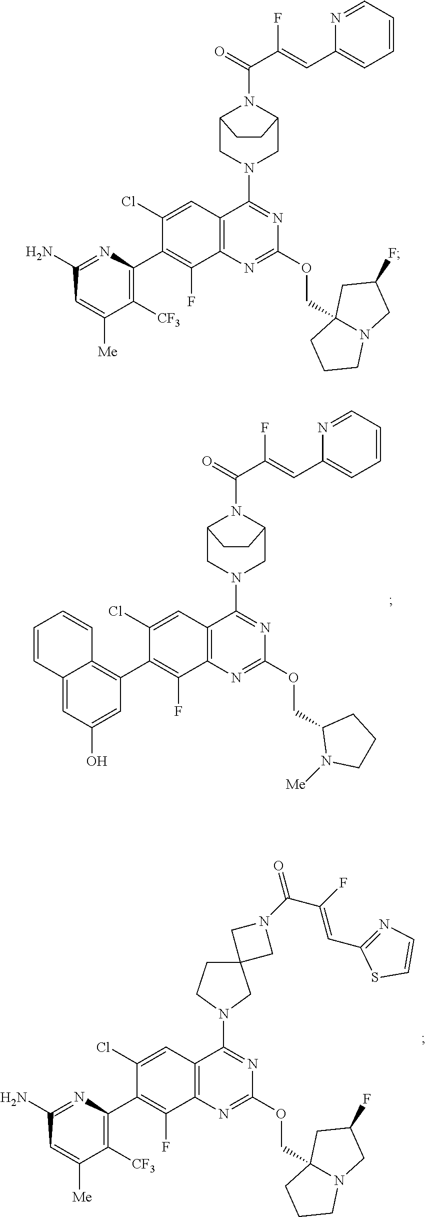 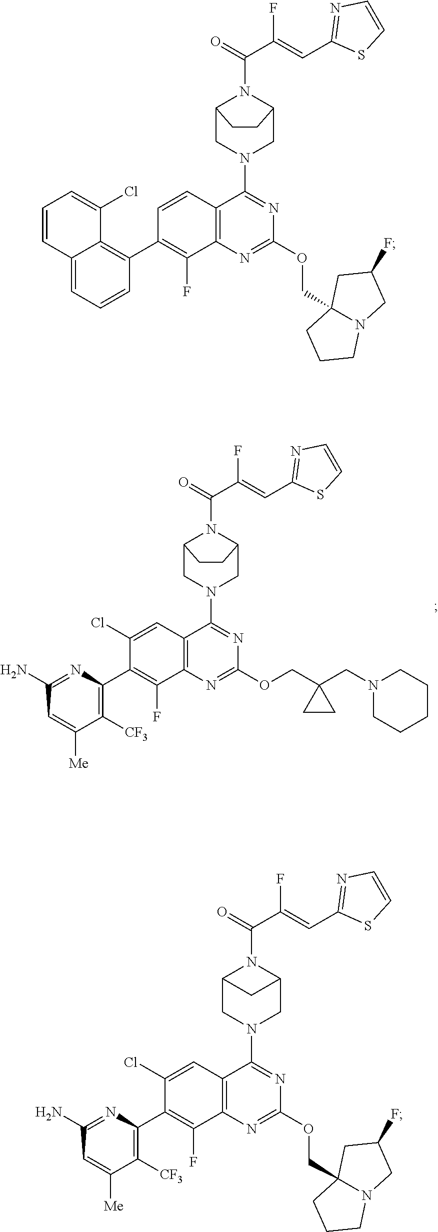 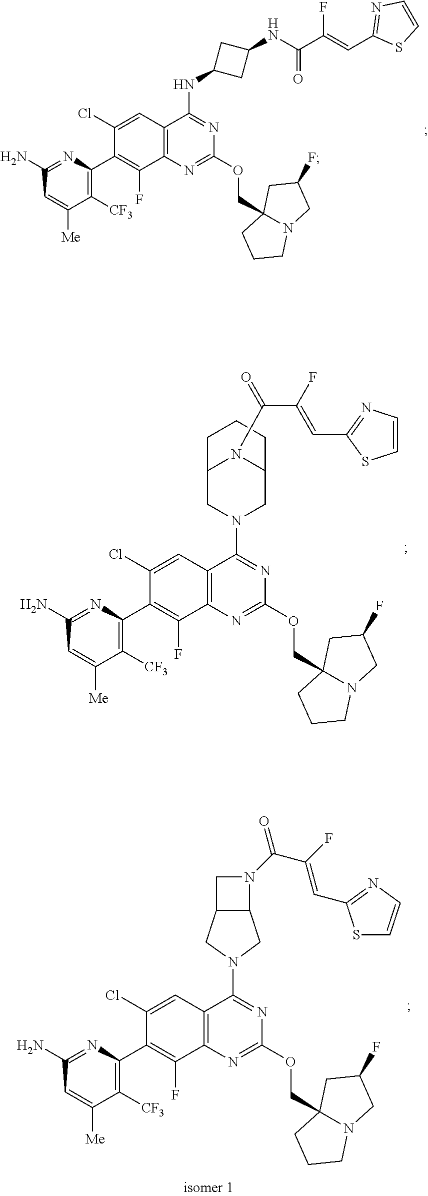 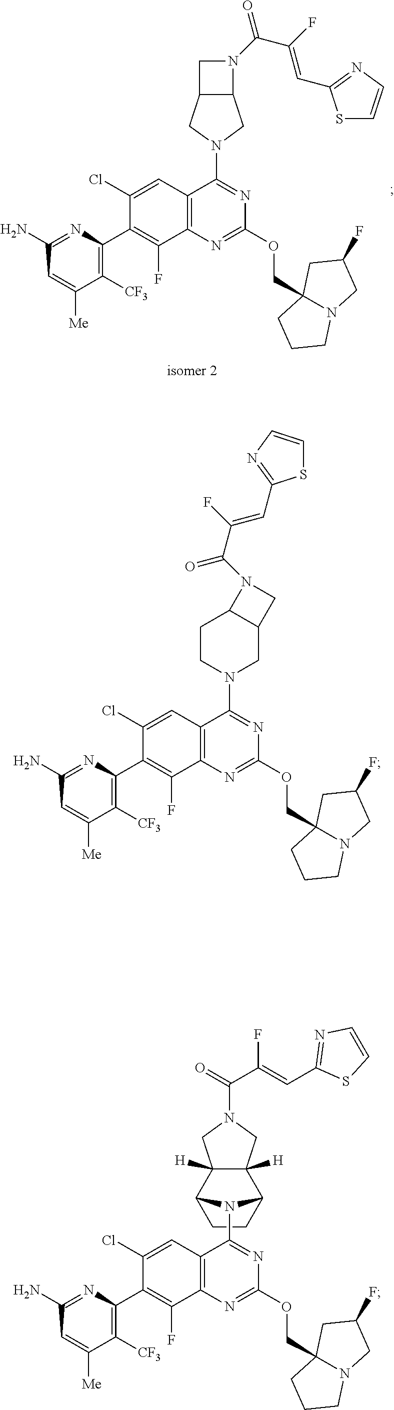 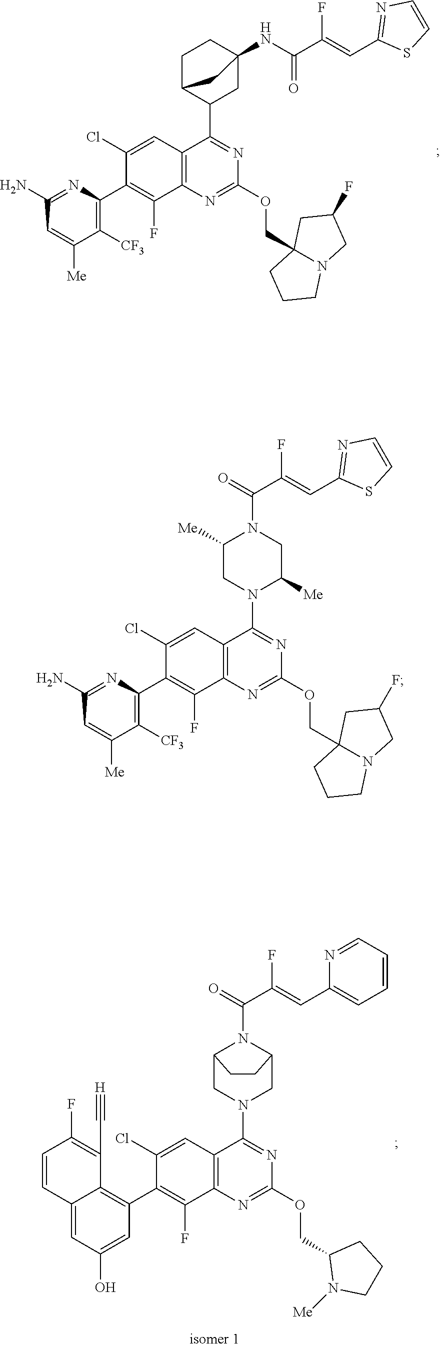 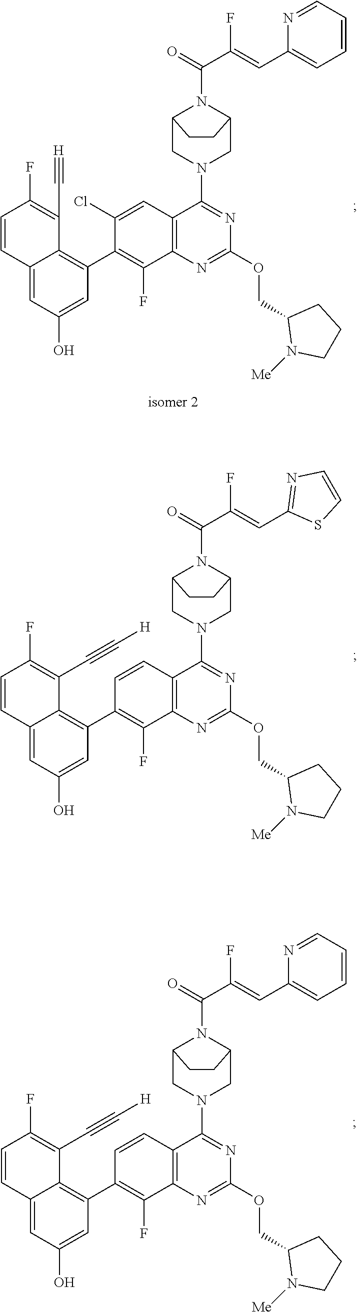 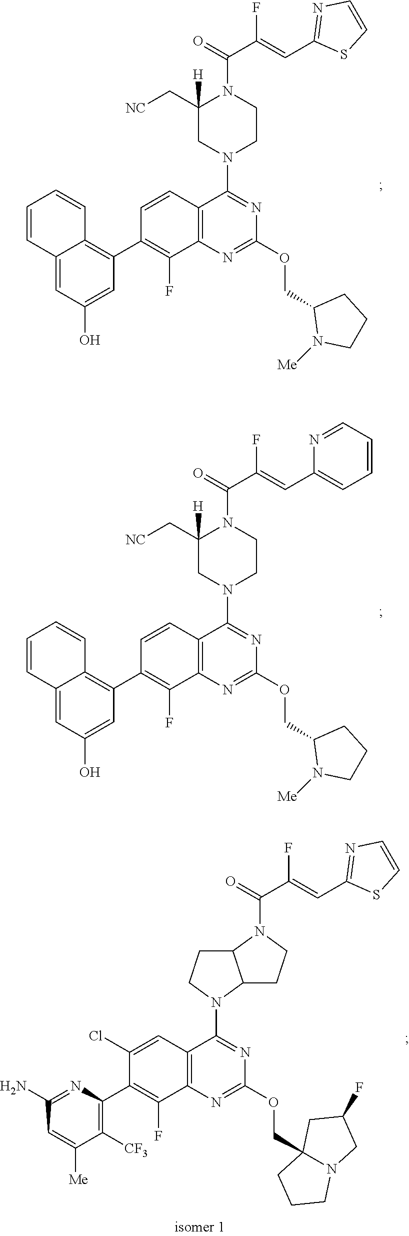 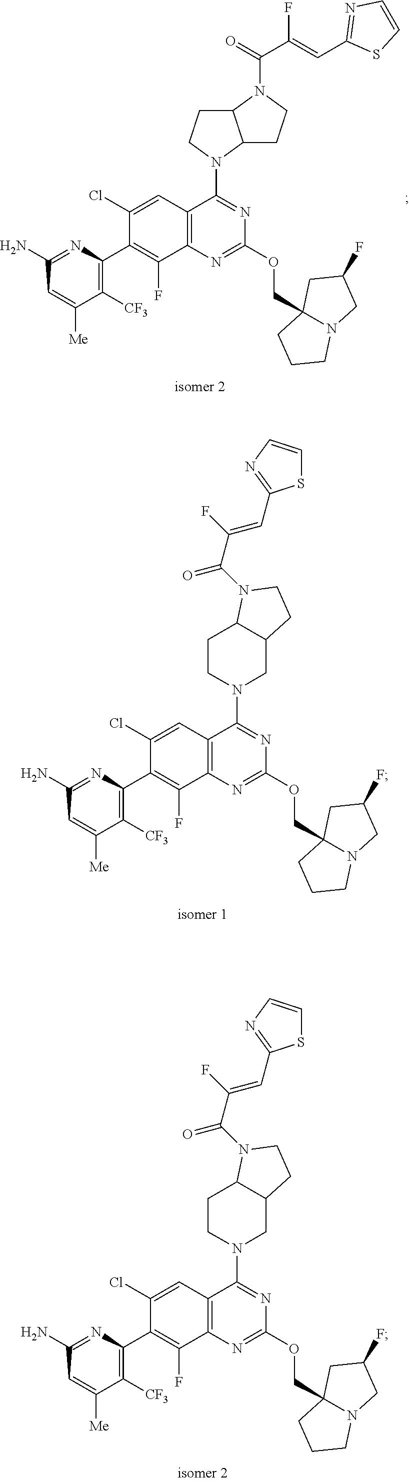 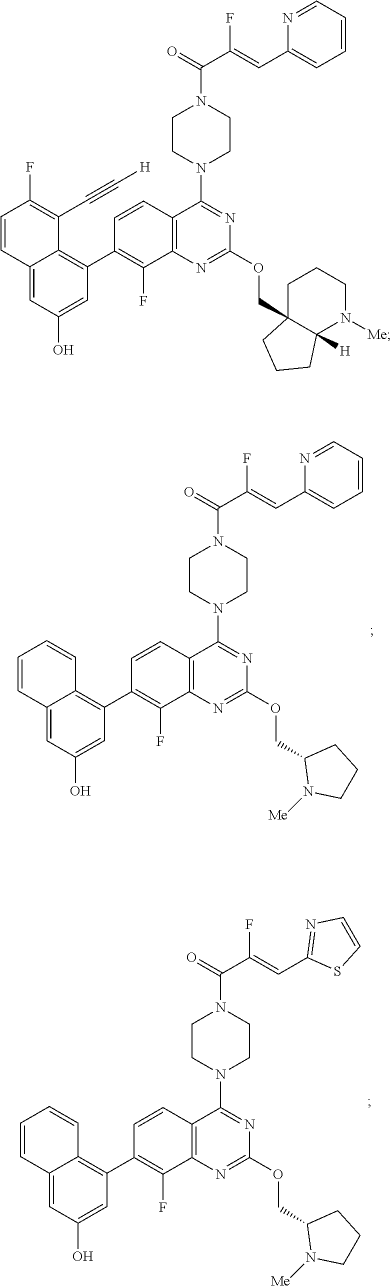 [SMILES: C#Cc1c(F)ccc2cc(O)cc(-c3ccc4c(N5CCN(C(=O)/C(F)=C/c6ccccn6)CC5)nc(OC[C@]56CCC[C@H]5N(C)CCC6)nc4c3F)c12 ; CN1CCC[C@H]1COc1nc(N2CCN(C(=O)/C(F)=C/c3ccccn3)CC2)c2ccc(-c3cc(O)cc4ccccc34)c(F)c2n1 ; CN1CCC[C@H]1COc1nc(N2CCN(C(=O)/C(F)=C/c3nccs3)CC2)c2ccc(-c3cc(O)cc4ccccc34)c(F)c2n1] 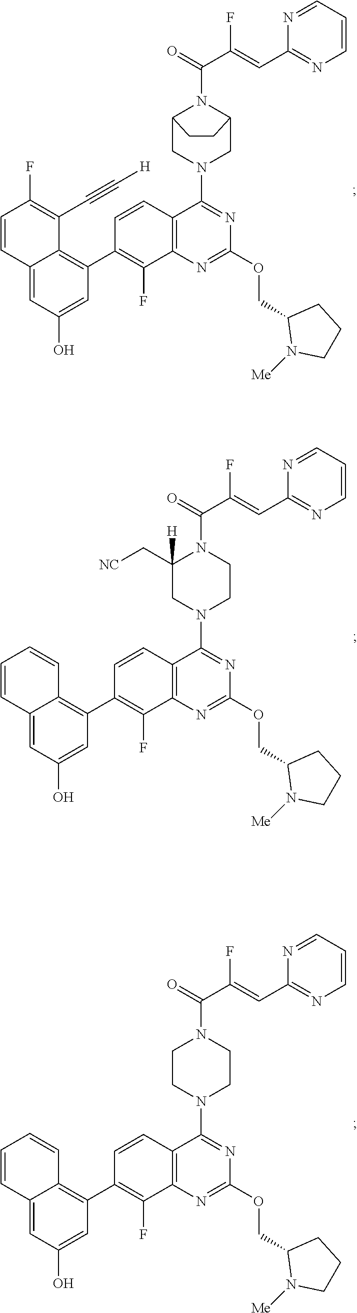 [SMILES: C#Cc1c(F)ccc2cc(O)cc(-c3ccc4c(N5CC6CCC(C5)N6C(=O)/C(F)=C/c5ncccn5)nc(OC[C@@H]5CCCN5C)nc4c3F)c12 ; CN1CCC[C@H]1COc1nc(N2CCN(C(=O)/C(F)=C/c3ncccn3)[C@@H](CC#N)C2)c2ccc(-c3cc(O)cc4ccccc34)c(F)c2n1 ; CN1CCC[C@H]1COc1nc(N2CCN(C(=O)/C(F)=C/c3ncccn3)CC2)c2ccc(-c3cc(O)cc4ccccc34)c(F)c2n1] 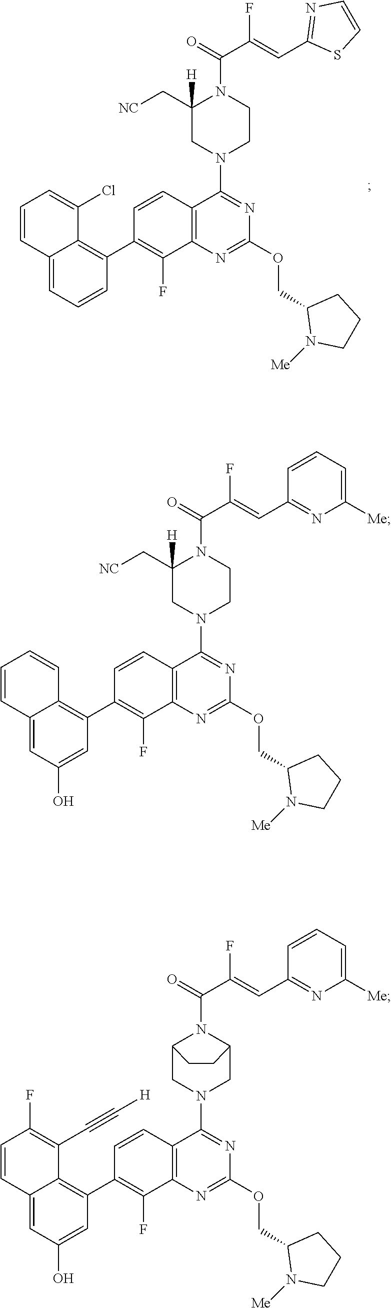 [SMILES: CN1CCC[C@H]1COc1nc(N2CCN(C(=O)/C(F)=C/c3nccs3)[C@@H](CC#N)C2)c2ccc(-c3cccc4cccc(Cl)c34)c(F)c2n1 ; Cc1cccc(/C=C(\F)C(=O)N2CCN(c3nc(OC[C@@H]4CCCN4C)nc4c(F)c(-c5cc(O)cc6ccccc56)ccc34)C[C@@H]2CC#N)n1 ; C#Cc1c(F)ccc2cc(O)cc(-c3ccc4c(N5CC6CCC(C5)N6C(=O)/C(F)=C/c5cccc(C)n5)nc(OC[C@@H]5CCCN5C)nc4c3F)c12] 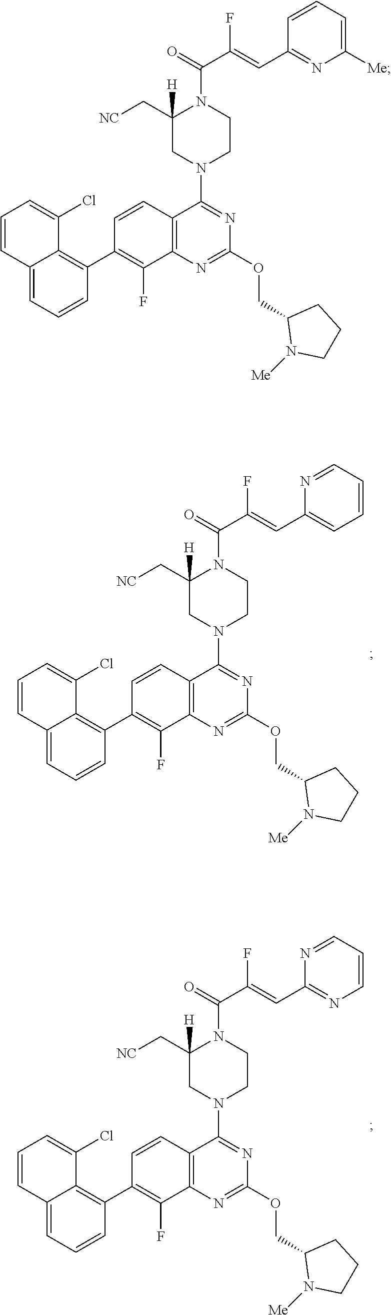 [SMILES: Cc1cccc(/C=C(\F)C(=O)N2CCN(c3nc(OC[C@@H]4CCCN4C)nc4c(F)c(-c5cccc6cccc(Cl)c56)ccc34)C[C@@H]2CC#N)n1 ; CN1CCC[C@H]1COc1nc(N2CCN(C(=O)/C(F)=C/c3ccccn3)[C@@H](CC#N)C2)c2ccc(-c3cccc4cccc(Cl)c34)c(F)c2n1 ; CN1CCC[C@H]1COc1nc(N2CCN(C(=O)/C(F)=C/c3ncccn3)[C@@H](CC#N)C2)c2ccc(-c3cccc4cccc(Cl)c34)c(F)c2n1] 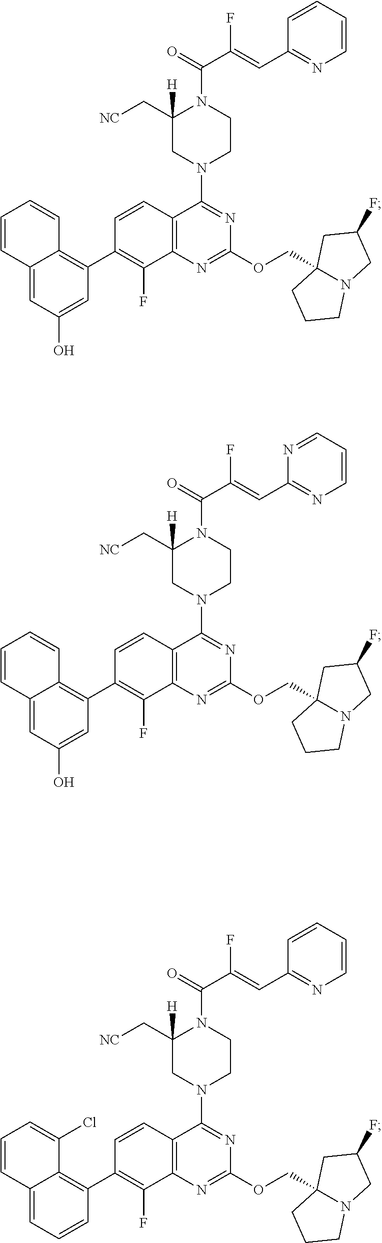 [SMILES: N#CC[C@H]1CN(c2nc(OC[C@@]34CCCN3C[C@H](F)C4)nc3c(F)c(-c4cc(O)cc5ccccc45)ccc23)CCN1C(=O)/C(F)=C/c1ccccn1 ; N#CC[C@H]1CN(c2nc(OC[C@@]34CCCN3C[C@H](F)C4)nc3c(F)c(-c4cc(O)cc5ccccc45)ccc23)CCN1C(=O)/C(F)=C/c1ncccn1 ; N#CC[C@H]1CN(c2nc(OC[C@@]34CCCN3C[C@H](F)C4)nc3c(F)c(-c4cccc5cccc(Cl)c45)ccc23)CCN1C(=O)/C(F)=C/c1ccccn1] 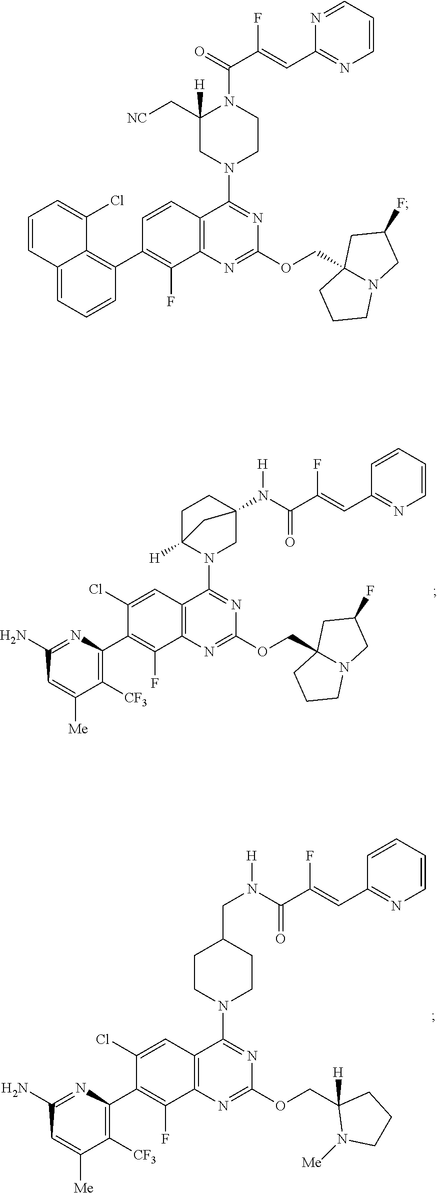 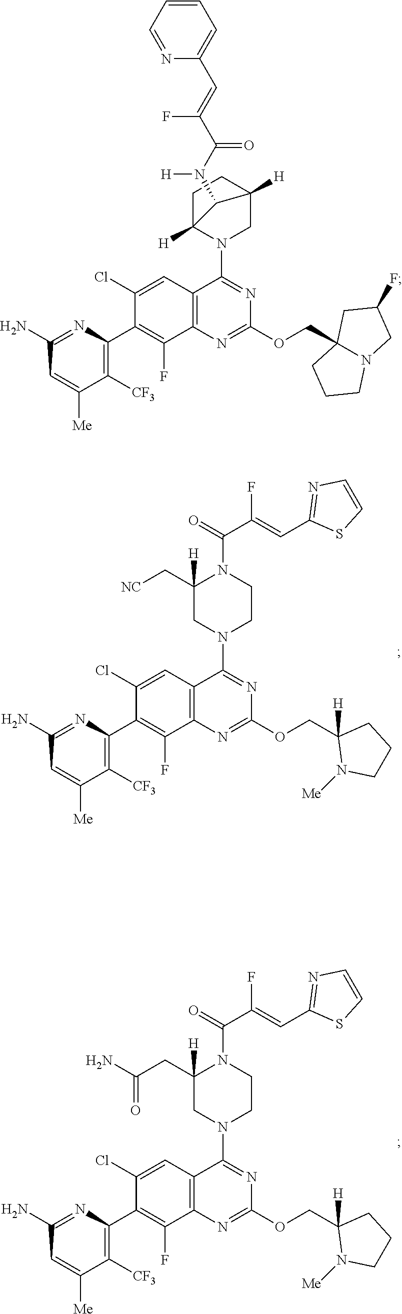 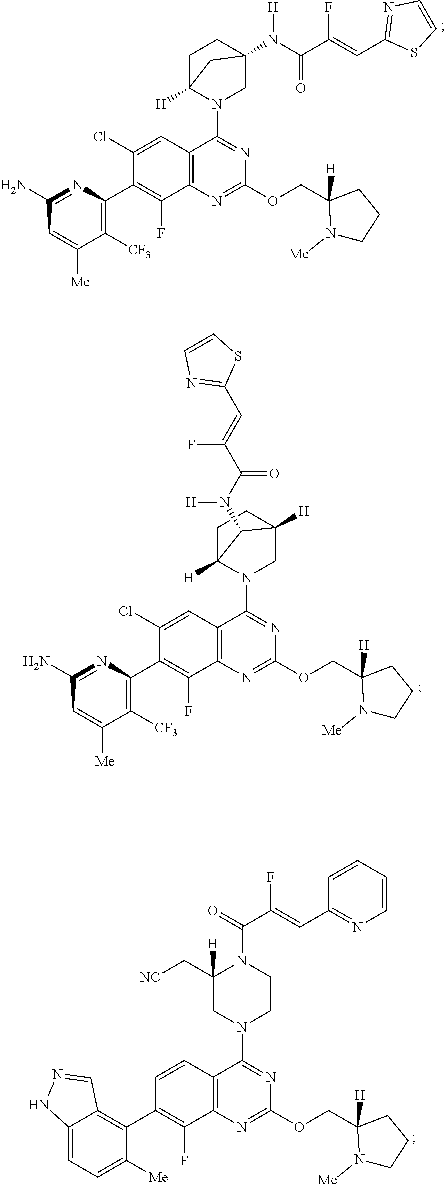 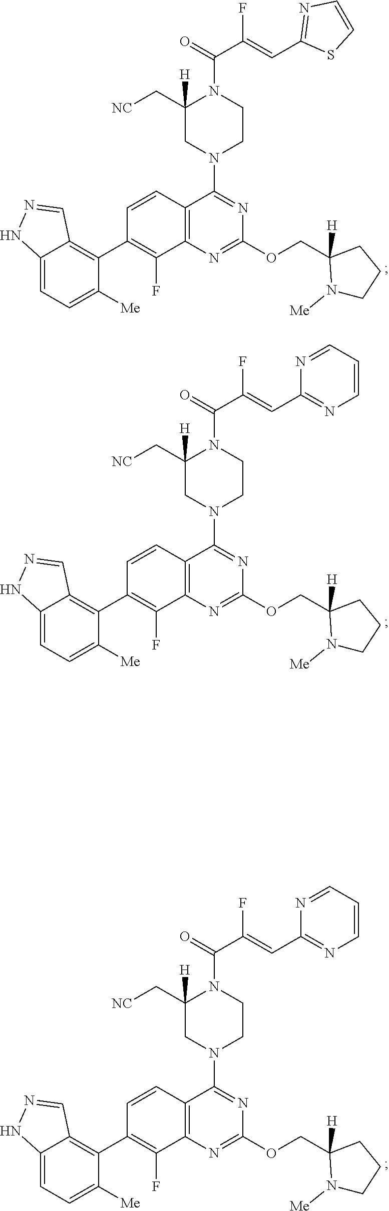 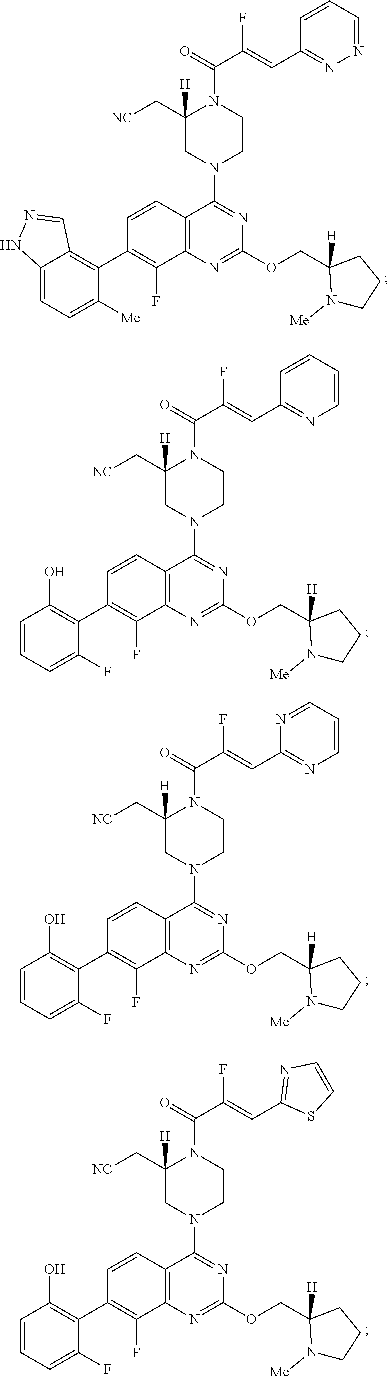 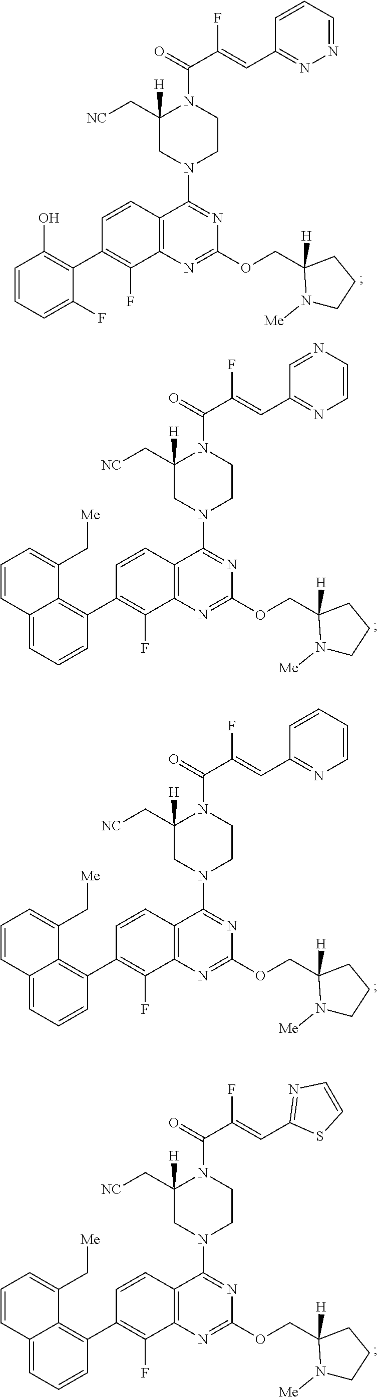 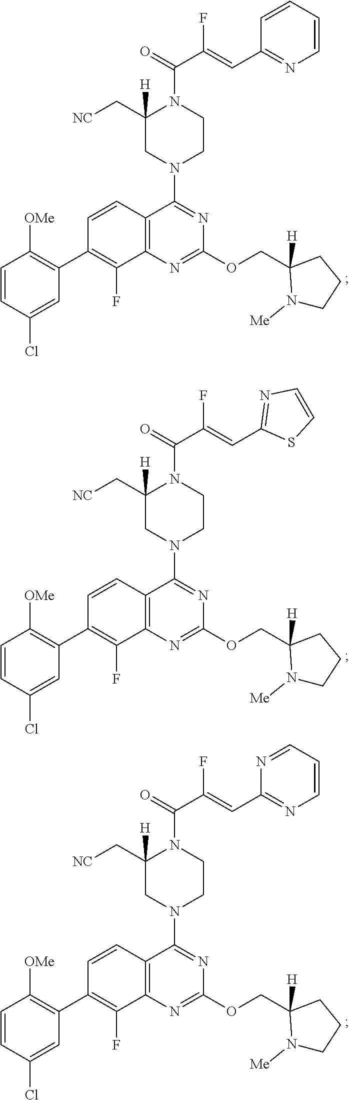 [SMILES: COc1ccc(Cl)cc1-c1ccc2c(N3CCN(C(=O)/C(F)=C/c4ccccn4)[C@@H](CC#N)C3)nc(OC[C@@H]3CCCN3C)nc2c1F ; COc1ccc(Cl)cc1-c1ccc2c(N3CCN(C(=O)/C(F)=C/c4nccs4)[C@@H](CC#N)C3)nc(OC[C@@H]3CCCN3C)nc2c1F ; COc1ccc(Cl)cc1-c1ccc2c(N3CCN(C(=O)/C(F)=C/c4ncccn4)[C@@H](CC#N)C3)nc(OC[C@@H]3CCCN3C)nc2c1F] 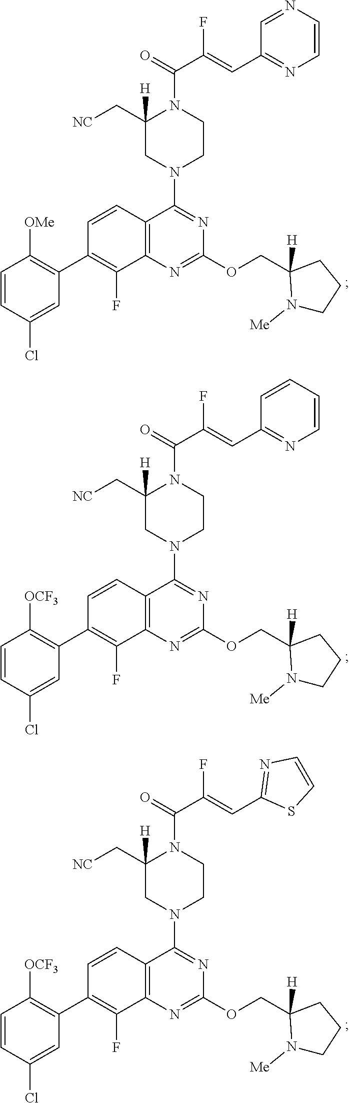 [SMILES: COc1ccc(Cl)cc1-c1ccc2c(N3CCN(C(=O)/C(F)=C/c4cnccn4)[C@@H](CC#N)C3)nc(OC[C@@H]3CCCN3C)nc2c1F ; CN1CCC[C@H]1COc1nc(N2CCN(C(=O)/C(F)=C/c3ccccn3)[C@@H](CC#N)C2)c2ccc(-c3cc(Cl)ccc3OC(F)(F)F)c(F)c2n1 ; CN1CCC[C@H]1COc1nc(N2CCN(C(=O)/C(F)=C/c3nccs3)[C@@H](CC#N)C2)c2ccc(-c3cc(Cl)ccc3OC(F)(F)F)c(F)c2n1] 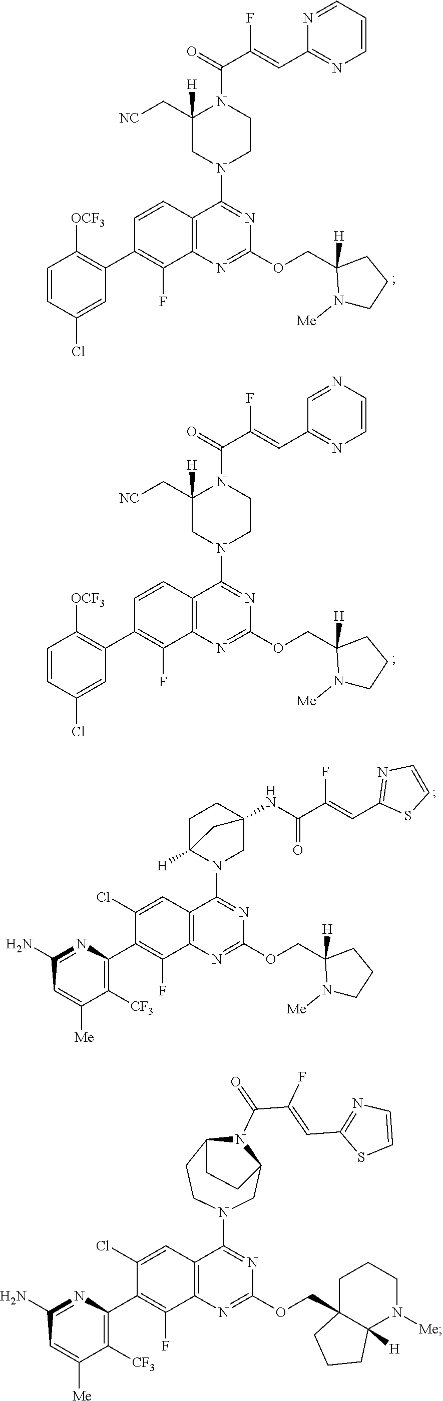 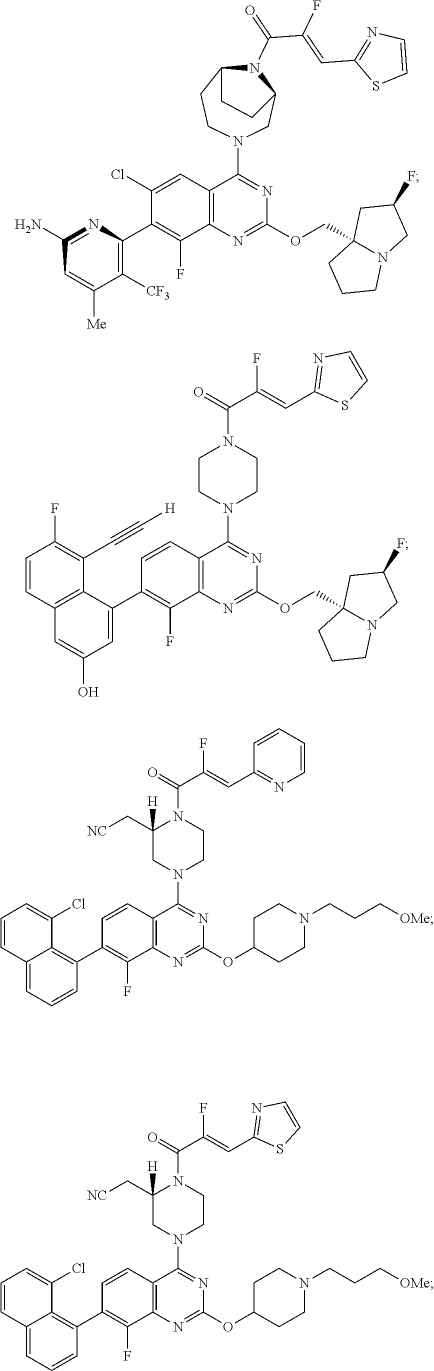 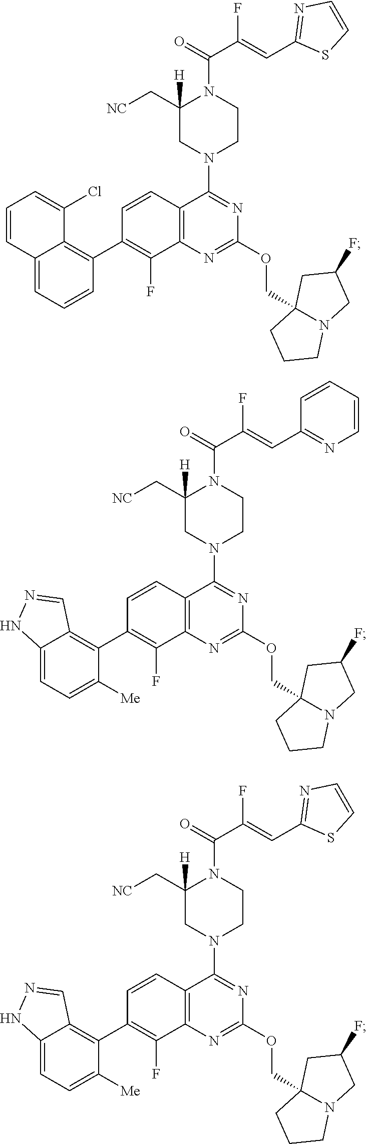 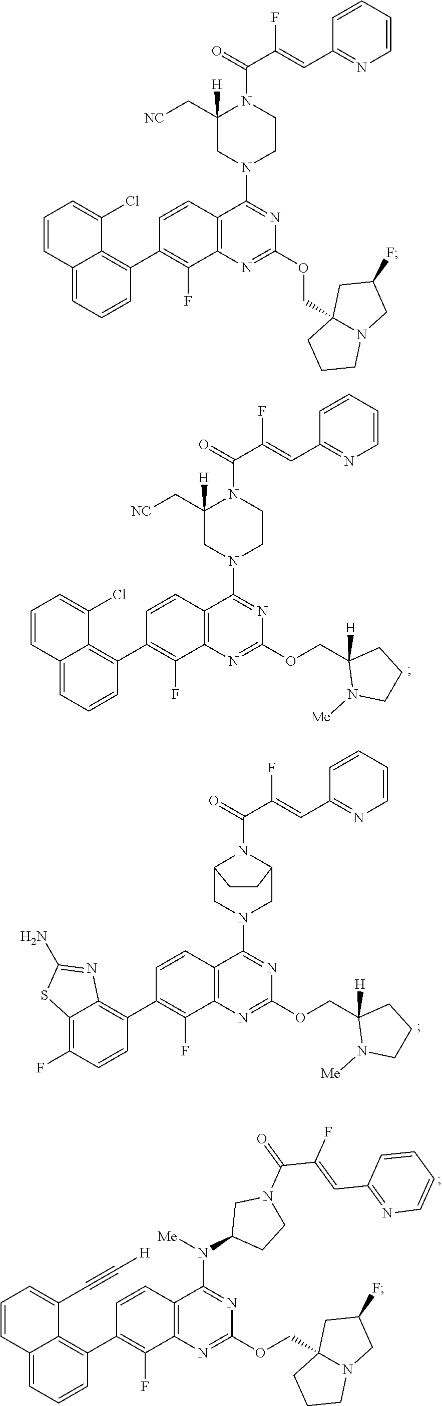 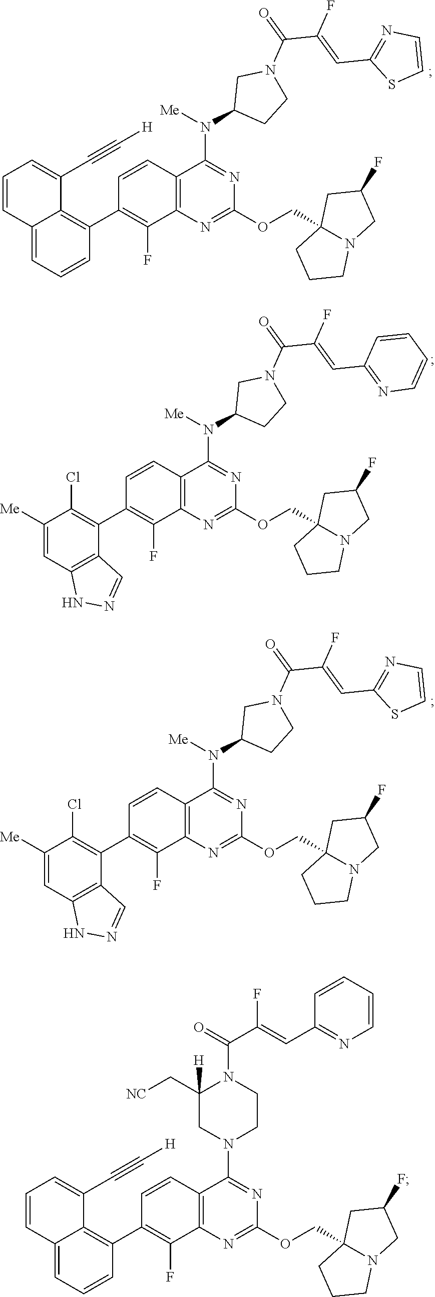 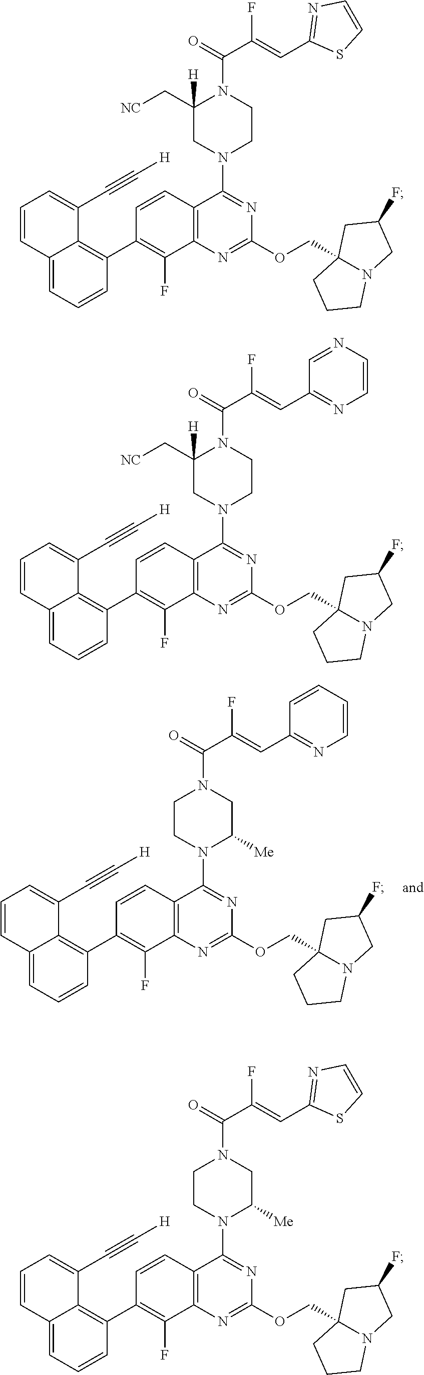
or a pharmaceutically acceptable salt thereof.

27. A compound selected from the group consisting of:
(Z)-1-((3S)-4-(6-chloro-7-(8-chloronaphthalen-1-yl)-8-fluoro-2-(((S)-1-methylpyrrolidin-2-yl)methoxy)quinazolin-4-yl)-3-methylpiperazin-1-yl)-2-fluoro-3-(thiazol-2-yl)prop-2-en-1-one;
(Z)-1-((3S)-4-(6-chloro-7-(8-chloronaphthalen-1-yl)-8-fluoro-2-(((S)-1-methylpyrrolidin-2-yl)methoxy)quinazolin-4-yl)-3-methylpiperazin-1-yl)-2-fluoro-3-(pyrimidin-2-yl)prop-2-en-1-one;
(Z)-1-((3S)-4-(6-chloro-7-(8-chloronaphthalen-1-yl)-8-fluoro-2-(((S)-1-methylpyrrolidin-2-yl)methoxy)quinazolin-4-yl)-3-methylpiperazin-1-yl)-2-fluoro-3-(pyridin-2-yl)prop-2-en-1-one;
(Z)-1-((1R,5S)-3-((7M)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one;
(Z)-1-((1R,5S)-3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,8-diazabicyclo[3.2.1]octan-8-yl)-2-fluoro-3-(pyridin-2-yl)prop-2-en-1-one;
(Z)-1-(5-((7M)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-2,5-diazabicyclo[2.2.2]octan-2-yl)-2-fluoro-3-(pyridin-2-yl)prop-2-en-1-one;
(Z)-1-((1R,5S)-3-((7M)-7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octan-8-yl)-2-fluoro-3-(pyridin-2-yl)prop-2-en-1-one;
(Z)-1-((1R,5S)-3-(6-chloro-8-fluoro-7-(3-hydroxynaphthalen-1-yl)-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one;
(Z)-1-(6-((7M)-7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-2,6-diazaspiro[3.4]octan-2-yl)-2-fluoro-3-(thiazol-2-yl)prop-2-en-1-one;
(Z)-1-((1R,5S)-3-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octan-8-yl)-2-fluoro-3-(thiazol-2-yl)prop-2-en-1-one;
(Z)-1-((1R,5S)-3-((7M)-7-(6-amino-4-methyl-3-(trifluoromethyl)pyridin-2-yl)-6-chloro-8-fluoro-2-((1-(piperidin-1-ylmethyl)cyclopropyl)methoxy)quinazolin-4-yl)-3,8-diazabicyclo[3.2.1]octan-8-yl)-2-fluoro-3-(thiazol-2-yl)prop-2-en-1-one;
(Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.1.1]heptan-6-yl)-2-fluoro-3-(thiazol-2-yl)prop-2-en-1-one;
(Z)-N-((1S,3s)-3-(((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)amino)cyclobutyl)-2-fluoro-3-(thiazol-2-yl)acrylamide;
(Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,9-diazabicyclo[3.3.1]nonan-9-yl)-2-fluoro-3-(thiazol-2-yl)prop-2-en-1-one;
(Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.2.0]heptan-6-yl)-2-fluoro-3-(thiazol-2-yl)prop-2-en-1-one (isomer 1);
(Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,6-diazabicyclo[3.2.0]heptan-6-yl)-2-fluoro-3-(thiazol-2-yl)prop-2-en-1-one (isomer 2);
(Z)-1-(3-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-3,7-diazabicyclo[4.2.0]octan-7-yl)-2-fluoro-3-(thiazol-2-yl)prop-2-en-1-one;
(Z)-1-((3aR,4S,7R,7aS)-8-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-2H-4,7-epiminoisoindol-2-yl)-2-fluoro-3-(thiazol-2-yl)prop-2-en-1-one;
(Z)-N-((1R,4R)-2-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)-2-azabicyclo[2.2.1]heptan-4-yl)-2-fluoro-3-(thiazol-2-yl)acrylamide;
(Z)-1-((2S,5R)-4-((7M)-7-(6-amino-4-methyl-3-(trifluoromethyl)pyridin-2-yl)-6-chloro-8-fluoro-2-((2-fluorotetrahydro-1H-pyrrolizin-7a(5H)-yl)methoxy)quinazolin-4-yl)-2,5-dimethylpiperazin-1-yl)-2-fluoro-3-(thiazol-2-yl)prop-2-en-1-one;
(2Z)-1-{3-[6-chloro-7-(8-ethynyl-7-fluoro-3-hydroxynaphthalen-1-yl)-8-fluoro-2-{[(2S)-1-methylpyrrolidin-2-yl]methoxy}quinazolin-4-yl]-3,8-diazabicyclo[3.2.1]octan-8-yl}-2-fluoro-3-(pyridin-2-yl)prop-2-en-1-one (isomer 1);
(2Z)-1-{3-[6-chloro-7-(8-ethynyl-7-fluoro-3-hydroxynaphthalen-1-yl)-8-fluoro-2-{[(2S)-1-methylpyrrolidin-2-yl]methoxy}quinazolin-4-yl]-3,8-diazabicyclo[3.2.1]octan-8-yl}-2-fluoro-3-(pyridin-2-yl)prop-2-en-1-one (isomer 2);
(Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(thiazol-2-yl)prop-2-en-1-one;
(Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one;
2-((S)-1-((Z)-2-fluoro-3-(thiazol-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile;
2-((S)-1-((Z)-2-fluoro-3-(pyridin-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile;
(Z)-1-(4-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)hexahydropyrrolo[3,2-b]pyrrol-1(2H)-yl)-2-fluoro-3-(thiazol-2-yl)prop-2-en-1-one (isomer 1);
(Z)-1-(4-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)hexahydropyrrolo[3,2-b]pyrrol-1(2H)-yl)-2-fluoro-3-(thiazol-2-yl)prop-2-en-1-one (isomer 2);
(Z)-1-(5-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-1H-pyrrolo[3,2-c]pyridin-1-yl)-2-fluoro-3-(thiazol-2-yl)prop-2-en-1-one (isomer 1);
(Z)-1-(5-((7M)-7-(6-amino-4-methyl-3-(trifluoromethyl)pyridin-2-yl)-6-chloro-8-fluoro-2-(((2R,7aR)-2-fluorotetrahydro-1H-pyrrolizin-7a(5H)-yl)methoxy)quinazolin-4-yl)octahydro-1H-pyrrolo[3,2-c]pyridin-1-yl)-2-fluoro-3-(thiazol-2-yl)prop-2-en-1-one (isomer 2);
(Z)-1-((1R,5S)-3-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)quinazolin-4-yl)-3,8-diazabicyclo[3.2.1]octan-8-yl)-2-fluoro-3-(pyridin-2-yl)prop-2-en-1-one;
(S,Z)-2-fluoro-1-(4-(8-fluoro-7-(3-hydroxynaphthalen-1-yl)-2-((1-methylpyrrolidin-2-yl)methoxy)quinazolin-4-yl)piperazin-1-yl)-3-(pyridin-2-yl)prop-2-en-1-one;
(S,Z)-2-fluoro-1-(4-(8-fluoro-7-(3-hydroxynaphthalen-1-yl)-2-((1-methylpyrrolidin-2-yl)methoxy)quinazolin-4-yl)piperazin-1-yl)-3-(thiazol-2-yl)prop-2-en-1-one;
(Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(pyrimidin-2-yl)prop-2-en-1-one;
2-((S)-1-((Z)-2-fluoro-3-(pyrimidin-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile;
(S,Z)-2-fluoro-1-(4-(8-fluoro-7-(3-hydroxynaphthalen-1-yl)-2-((1-methylpyrrolidin-2-yl)methoxy)quinazolin-4-yl)piperazin-1-yl)-3-(pyrimidin-2-yl)prop-2-en-1-one;
2-((S)-1-((Z)-2-fluoro-3-(thiazol-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile;
2-((S)-1-((Z)-2-fluoro-3-(6-methylpyridin-2-yl)acryloyl)-4-(8-fluoro-7-(3-hydroxynaphthalen-1-yl)-2-(((S)-1-methylpyrrolidin-2-yl)methoxy)quinazolin-4-yl)piperazin-2-yl)acetonitrile;
(Z)-1-((1R,5S)-3-(7-(8-ethynyl-7-fluoro-3-hydroxynaphthalen-1-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(6-methylpyridin-2-yl)prop-2-en-1-one;
2-((S)-1-((Z)-2-fluoro-3-(6-methylpyridin-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile;
2-((S)-1-((Z)-2-fluoro-3-(pyridin-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile;
2-((S)-1-((Z)-2-fluoro-3-(pyrimidin-2-yl)acryloyl)-4-(8-fluoro-7-(8-chloronaphthalen-1-yl)-2-(((S)-1-methylpyrrolidin-2-yl)methoxy)quinazolin-4-yl)piperazin-2-yl)acetonitrile;
2-((S)-4-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-1-((Z)-2-fluoro-3-(pyridin-2-yl)acryloyl)piperazin-2-yl)acetonitrile;
2-((S)-4-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-1-((Z)-2-fluoro-3-(pyrimidin-2-yl)acryloyl)piperazin-2-yl)acetonitrile;
2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyridin-2-yl)acryloyl)piperazin-2-yl)acetonitrile;
2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyrimidin-2-yl)acryloyl)piperazin-2-yl)acetonitrile;
(2Z)-N-[(1S,4S)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-4-yl]-2-fluoro-3-(pyridin-2-yl)prop-2-enamide;
(2Z)-N-({1-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]piperidin-4-yl}methyl)-2-fluoro-3-(pyridin-2-yl)prop-2-enamide;
(2Z)-N-[(1R,4R,7R)-2-[(7M)-2-{[(2R,7aR)-2-fluoro-hexahydro-1H-pyrrolizin-7a-yl]methoxy}-7-[6-amino-4-methyl-3-(trifluoromethyl)pyridin-2-yl]-6-chloro-8-fluoroquinazolin-4-yl]-2-azabicyclo[2.2.1]heptan-7-yl]-2-fluoro-3-(pyridin-2-yl)prop-2-enamide
2-[(2S)-4-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-1-[(2Z)-2-fluoro-3-(thiazol-2-yl)prop-2-enoyl]piperazin-2-yl]acetonitrile
2-[(2S)-4-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-1-[(2Z)-2-fluoro-3-(thiazol-2-yl)prop-2-enoyl]piperazin-2-yl]acetamide
(2Z)-N-[(1S,4S)-2-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-2-azabicyclo[2.2.1]heptan-4-yl]-2-fluoro-3-(thiazol-2-yl)prop-2-enamide;
(2Z)-N-[(1R,4R,7R)-2-[(7M)-7-[6-amino-4-methyl-3-(trifluoromethyl)pyridin-2-yl]-6-chloro-8-fluoro-2-{[(2S)-1-methylpyrrolidin-2-yl]methoxy}quinazolin-4-yl]-2-azabicyclo[2.2.1]heptan-7-yl]-2-fluoro-3-(1,3-thiazol-2-yl)prop-2-enamide;
2-((2S)-1-((Z)-2-fluoro-3-(pyridin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile;
2-((2S)-1-((Z)-2-fluoro-3-(thiazol-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile;
2-((2S)-1-((Z)-2-fluoro-3-(pyrimidin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile;
2-((2S)-1-((Z)-2-fluoro-3-(pyrimidin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile;
2-((2S)-1-((Z)-2-fluoro-3-(pyridazin-2-yl)acryloyl)-4-(8-fluoro-7-(5-methyl-1H-indazol-4-yl)-2-(((S)-1-methylpyrrolidin-2-yl)methoxy)quinazolin-4-yl)piperazin-2-yl)acetonitrile;
2-((2S)-1-((Z)-2-fluoro-3-(pyridin-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile;
2-((2S)-1-((Z)-2-fluoro-3-(pyrimidin-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile;
2-((2S)-1-((Z)-2-fluoro-3-(thiazol-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile;
2-((2S)-1-((Z)-2-fluoro-3-(pyridazin-2-yl)acryloyl)-4-(8-fluoro-7-(2-fluoro-6-hydroxyphenyl)-2-(((S)-1-methylpyrrolidin-2-yl)methoxy)quinazolin-4-yl)piperazin-2-yl)acetonitrile;
2-((S)-4-(7-(8-ethylnaphthalen-1-yl)-8-fluoro-2-(((S)-1-methylpyrrolidin-2-yl)methoxy)quinazolin-4-yl)-1-((Z)-2-fluoro-3-(pyrazin-2-yl)acryloyl)piperazin-2-yl)acetonitrile;
2-((S)-4-(7-(8-ethylnaphthalen-1-yl)-8-fluoro-2-(((S)-1-methylpyrrolidin-2-yl)methoxy)quinazolin-4-yl)-1-((Z)-2-fluoro-3-(pyridin-2-yl)acryloyl)piperazin-2-yl)acetonitrile;
2-((S)-4-(7-(8-ethylnaphthalen-1-yl)-8-fluoro-2-(((S)-1-methylpyrrolidin-2-yl)methoxy)quinazolin-4-yl)-1-((Z)-2-fluoro-3-(thiazol-2-yl)acryloyl)piperazin-2-yl)acetonitrile;
2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(pyridin-2-yl)acryloyl)piperazin-2-yl)acetonitrile;
2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(thiazol-2-yl)acryloyl)piperazin-2-yl)acetonitrile;
2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(pyrimidin-2-yl)acryloyl)piperazin-2-yl)acetonitrile;
2-((S)-4-(7-(5-chloro-2-methoxyphenyl)-8-fluoro-2-(((S)-1-methylpyrrolidin-2-yl)methoxy)quinazolin-4-yl)-1-((Z)-2-fluoro-3-(piperazin-2-yl)acryloyl)piperazin-2-yl)acetonitrile;
2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(pyridin-2-yl)acryloyl)piperazin-2-yl)acetonitrile;
2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(thiazol-2-yl)acryloyl)piperazin-2-yl)acetonitrile;
2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(pyrimidin-2-yl)acryloyl)piperazin-2-yl)acetonitrile;
2-((S)-4-(7-(5-chloro-2-(trifluoromethoxy)phenyl)-8-fluoro-2-(((S)-1-methylpyrrolidin-2-yl)methoxy)quinazolin-4-yl)-1-((Z)-2-fluoro-3-(pyrazin-2-yl)acryloyl)piperazin-2-yl)acetonitrile;
(Z)-N-((1S,4S)-2-(7M-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-2-azabicyclo[2.2.1]heptan-4-yl)-2-fluoro-3-(thiazol-2-yl)acrylamide;
(Z)-1-((1R,6S)-3-(7M-(6-amino-4-methyl-3-(trifluoromethyl)pyridin-2-yl)-6-chloro-8-fluoro-2-(((4aS,7aR)-1-methyloctahydro-4aH-cyclopenta[b]pyridin-4a-yl)methoxy)quinazolin-4-yl)-3,9-diazabicyclo[4.2.1]nonan-9-yl)-2-fluoro-3-(thiazol-2-yl)prop-2-en-1-one;
(Z)-1-((1R,6S)-3-(7M-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,9-diazabicyclo[4.2.1]nonan-9-yl)-2-fluoro-3-(thiazol-2-yl)prop-2-en-1-one;
(Z)-1-(4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)piperazin-1-yl)-2-fluoro-3-(thiazol-2-yl)prop-2-en-1-one;
(S,Z)-2-(4-(7-(8-chloronaphthalen-1-yl)-8-fluoro-2-((1-(3-methoxypropyl)piperidin-4-yl)oxy)quinazolin-4-yl)-1-(2-fluoro-3-(pyridin-2-yl)acryloyl)piperazin-2-yl)acetonitrile;
(S,Z)-2-(4-(7-(8-chloronaphthalen-1-yl)-8-fluoro-2-((1-(3-methoxypropyl)piperidin-4-yl)oxy)quinazolin-4-yl)-1-(2-fluoro-3-(thiazol-2-yl)acryloyl)piperazin-2-yl)acetonitrile;
2-((S)-4-(7-(8-chloronaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(thiazol-2-yl)acryloyl)piperazin-2-yl)acetonitrile;
2-((2S)-4-(8-fluoro-2-(((2R,7aS)-2-fluorotetrahydro-1H-pyrrolizin-7a(5H)-yl)methoxy)-7-(5-methyl-1H-indazol-4-yl)quinazolin-4-yl)-1-((Z)-2-fluoro-3-(pyridin-2-yl)acryloyl)piperazin-2-yl)acetonitrile;
2-((2S)-4-(8-fluoro-2-(((2R,7aS)-2-fluorotetrahydro-1H-pyrrolizin-7a(5H)-yl)methoxy)-7-(5-methyl-1H-indazol-4-yl)quinazolin-4-yl)-1-((Z)-2-fluoro-3-(thiazol-2-yl)acryloyl)piperazin-2-yl)acetonitrile;
8-(4-((S)-3-(cyanomethyl)-4-((Z)-2-fluoro-3-(pyridin-2-yl)acryloyl)piperazin-1-yl)-8-fluoro-2-(((2R,7aS)-2-fluorotetrahydro-1H-pyrrolizin-7a(5H)-yl)methoxy)quinazolin-7-yl)-1-naphthonitrile;
8-(4-((S)-3-(cyanomethyl)-4-((Z)-2-fluoro-3-(pyridin-2-yl)acryloyl)piperazin-1-yl)-8-fluoro-2-(((S)-1-methylpyrrolidin-2-yl)methoxy)quinazolin-7-yl)-1-naphthonitrile;
(Z)-1-((1R,5S)-3-(7-(2-amino-7-fluorobenzo[d]thiazol-4-yl)-8-fluoro-2-(((S)-1-methylpyrrolidin-2-yl)methoxy)quinazolin-4-yl)-3,8-diazabicyclo[3.2.1]octan-8-yl)-2-fluoro-3-(pyridin-2-yl)prop-2-en-1-one;
(Z)-1-((R)-3-((7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(pyridin-2-yl)prop-2-en-1-one;
(Z)-1-((R)-3-((7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(thiazol-2-yl)prop-2-en-1-one;
(Z)-1-((3R)-3-((7-(5-chloro-6-methyl-1H-indazol-4-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(pyridin-2-yl)prop-2-en-1-one;
(Z)-1-((3R)-3-((7-(5-chloro-6-methyl-1H-indazol-4-yl)-8-fluoro-2-(((2R,7aS)-2-fluorotetrahydro-1H-pyrrolizin-7a(5H)-yl)methoxy)quinazolin-4-yl)(methyl)amino)pyrrolidin-1-yl)-2-fluoro-3-(thiazol-2-yl)prop-2-en-1-one;
2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyridin-2-yl)acryloyl)piperazin-2-yl)acetonitrile;
2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(thiazol-2-yl)acryloyl)piperazin-2-yl)acetonitrile;
2-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-1-((Z)-2-fluoro-3-(pyrazin-2-yl)acryloyl)piperazin-2-yl)acetonitrile;
(Z)-1-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-3-methylpiperazin-1-yl)-2-fluoro-3-(pyridin-2-yl)prop-2-en-1-one; and
(Z)-1-((S)-4-(7-(8-ethynylnaphthalen-1-yl)-8-fluoro-2-(((2R,7aS)-2-fluorotetrahydro-1H-pyrrolizin-7a(5H)-yl)methoxy)quinazolin-4-yl)-3-methylpiperazin-1-yl)-2-fluoro-3-(thiazol-2-yl)prop-2-en-1-one;
or a pharmaceutically acceptable salt thereof.

28. A pharmaceutical composition comprising a compound of any one of claims 1 to 27, or a pharmaceutically acceptable salt thereof, and one or more pharmaceutically acceptable excipients.

29. An oral dosage form comprising a compound of any one of claims 1 to 27, or a pharmaceutically acceptable salt thereof, and one or more pharmaceutically acceptable excipients.

30. A method of treating cancer expressing KRAS G12C, G12D and/or G12V mutation in a subject in need thereof, the method comprising administering to the subject a compound of any one of claims 1 to 27, or a pharmaceutically acceptable salt thereof.

31. A method of treating cancer expressing KRAS G12C mutation in a subject in need thereof, the method comprising administering to the subject a compound of any one of claims 1 to 27, or a pharmaceutically acceptable salt thereof.

32. A method for treating a cancer susceptible to KRAS G12C inhibition in a subject in need thereof, the method comprising administering to the subject a compound of any one of claims 1 to 27, or a pharmaceutically acceptable salt thereof.

33. A method for treating a cancer in a subject in need thereof, the method comprising administering to the subject a compound of any one of claims 1 to 27, or a pharmaceutically acceptable salt thereof, wherein the cancer is lung cancer, colorectal cancer, pancreatic cancer, breast cancer, bladder cancer, cervical cancer, ovarian cancer, gastric cancer or cancer of the uterus.

34. A method for treating a cancer in a subject in need thereof, the method comprising administering to the subject a compound of any one of claims 1 to 27, or a pharmaceutically acceptable salt thereof, wherein the cancer is non-small cell lung cancer.
